(19) United States
(12) Patent Application Publication
(10) Pub. No.: US 2026/0049060 A1
(43) Pub. Date: Feb. 19, 2026

(54) TETRAHYDROISOQUINOLINYLMETHYLBENZAMIDE COMPOUNDS

(71) Applicant: National Health Research Institutes, Miaoli County (TW)

(72) Inventors: Shau-Hua Ueng, Miaoli County (TW); Shiu-Hwa Yeh, Miaoli County (TW); Chuan Shih, Westfield, IN (US)

(21) Appl. No.: 19/303,279

(22) Filed: Aug. 18, 2025

Related U.S. Application Data
(60) Provisional application No. 63/684,830, filed on Aug. 19, 2024.

(51) Int. Cl. C07D 217/18; A61K 31/472; A61K 31/4725; A61P 29/00; C07D 217/16; C07D 401/12; C07D 405/12; C07D 409/12

(52) U.S. Cl. CPC C07D 217/18; A61K 31/472; A61K 31/4725; A61P 29/00; C07D 217/16; C07D 401/12; C07D 405/12; C07D 409/12

(57) ABSTRACT

Disclosed are tetrahydroisoquinoline compounds useful for treating an opioid receptor-associated condition including pain, immune disease, esophageal reflux, diarrhea, anxiety, or heroin addiction. Also provided are pharmaceutical compositions and treatment methods.

### CROSS REFERENCE TO RELATED APPLICATION

This application claims the benefit of and priority to U.S. Provisional Application No. 63/684,830 filed Aug. 19, 2024, the entire content of which is incorporated by reference.

### BACKGROUND OF THE INVENTION

Opioids act in both central and peripheral nervous systems to produce various pharmacological effects including analgesia and decreased gastrointestinal motility. They have long been used as the most effective analgesics for treating acute pain (e.g., post-operative pain) and chronic pain (e.g., cancer pain). See, e.g., Waldhoer et al., Annu Rev Biochem, 2004, 73:953-990.

Opioids primarily activate three classic subtypes of opioid receptors, namely, mu-opioid receptor (MOR), delta-opioid receptor (DOR), kappa-opioid receptor (KOR), and non-classic nociception receptor (NOP). Activation of NOP receptor has been suggested to be an alternative strategy for inhibiting the opioid related side effects and treating pain. See, e.g., Hu et al., Pain, 2010, 148:107-113; and Ding et al., Sci Transl Med, 2018, 10: eaar3483. Various heterocyclic compounds have been used as MOR agonists for treating an opioid receptor-associated condition, e.g., pain, immune function, esophageal reflux, and cough. Yet, known heterocyclic compounds typically produce adverse effects, such as respiratory depression. Moreover, long-term use of these compounds for controlling chronic pain develops severe side effects such as tolerance, dependence, and addiction. See, e.g., Tao et al., J Pharmacol Exp Ther, 2002, 303:549-556.

There is a need to develop new MOR modulators that have fewer and/or less deterious side effects for therapeutic uses.

### SUMMARY OF THE INVENTION

One aspect of the invention relates to tetrahydroisoquinolinylmethylbenzamide compounds as MOR agonists for treating an opioid receptor-associated condition.

It is unexpectedly found that these tetrahydroisoquinolinylmethylbenzamide compounds are potent in activating a MOR and have much less deterious side effects.

The tetrahydroisoquinolinylmethylbenzamide compounds include compounds of Formula (I), (II), (III), (IV), and (V) below.

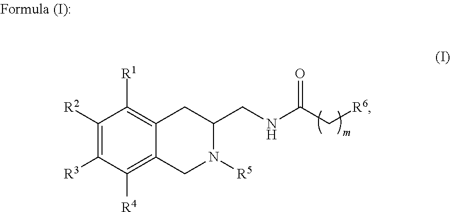

in which
each of R¹-R⁴, independently, is H, halo, C₁₋₆ alkyl, C₁₋₆ haloalkyl, C₁₋₆ alkoxy;
R⁵ is C₁₋₆ alkyl, C₃₋₈ cycloalkyl, C₁₋₆ alkenyl, C₁₋₆ alkoxy, C₁₋₆ alkenylether;
R⁶ is C₁₋₆ alkyl, C₃₋₈ cycloalkyl, C₂₋₈ heterocycloalkyl, C₆₋₁₄ aryl, fused C₆₋₁₄ cycloalkylaryl, C₆₋₁₄ aralkyl, C₂₋₁₃ heteroaryl, or fused C₂₋₁₃ heterocycloalkylaryl; and
m is 0, 1 or 2.

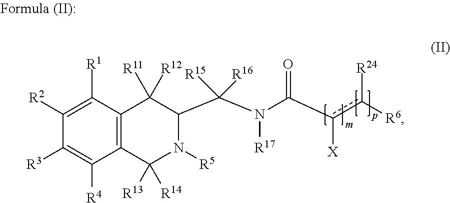

in which
each of R¹ to R⁴, independently, is H, halo, cyano, amino, nitro, thio, C₁₋₆ alkyl, or C₁₋₆ alkoxy;
R⁵ is C₁₋₆ alkyl, C₃₋₈ cycloalkyl, or C₁₋₆ alkenyl;
R⁶ is C₁₋₆ alkyl, C₃₋₈ cycloalkyl, C₂₋₈ heterocycloalkyl, C₆₋₁₄ aryl, C₆₋₁₄ cycloalkylaryl, C₆₋₁₄ aralkyl, C₂₋₁₃ heteroaryl, or 6 to 16-membered heterocycloalkylaryl;
each of R¹¹ to R¹⁶, independently, is H, halo, hydroxyl, cyano, amino, nitro, thio, carboxyl, C₁₋₆ alkyl, or C₁₋₆ alkoxy;
R¹⁷ is H, C₁₋₆ alkyl, C₃₋₈ cycloalkyl, or C₁₋₆ alkenyl;
X is absent, H, or CR¹⁸R¹⁹R²⁰, or X and R⁶, together with the carbon atoms they bond to, form a five or six-membered cycloalkyl- or heterocycloalkyl-fused phenyl, or X and R²⁴, together with the carbon atoms they bond to, form cyclopropylene;
each of R¹⁸ to R²⁰, independently, is H, halo, hydroxyl, thio, cyano, amino, nitro, carboxyl, C₁₋₆ alkyl, or C₁₋₆ alkoxy;
R²⁴ is absent, H, halo, hydroxyl, cyano, amino, nitro, thio, carboxyl, C₁₋₆ alkyl, C₃₋₈ cycloalkyl, or C₁₋₆ alkoxy;
each of amino, thio, alkyl, alkoxy, and alkenyl is unsubstituted or substituted with one or more of halo, hydroxyl, thio, cyano, amino, nitro, carboxyl, and aryl;
each of cycloalkyl, heterocycloalkyl, cycloalkylaryl, aralkyl, heteroaryl, heterocycloalkylaryl, and cyclopropylene is unsubstituted or substituted with one or more of halo, hydroxyl, thio, cyano, amino, nitro, carboxyl, alkyl, alkoxy, and aryl;
aryl is unsubstituted or substituted with one or more of halo, thio, cyano, amino, nitro, carboxyl, alkyl, and aryl;
each of the five or six-membered cycloalkyl- or heterocycloalkyl-fused phenyl is unsubstituted or substituted with one or more of halo, hydroxyl, thio, cyano, amino, nitro, carboxyl, alkyl, alkoxy, and aryl;
each of m and n, independently, is 0 or 1; and
[symbol] is a single bond or double bond.

The compounds of Formula (III) are a subset of the compounds of Formula (II) above:

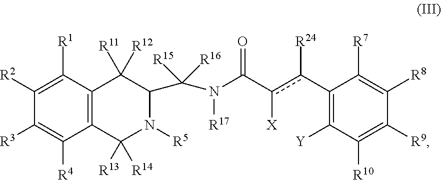

in which
each of R⁷ to R¹⁰, independently, is H, halo, thio, cyano, amino, nitro, carboxyl, or C₁₋₆ alkyl;
R¹⁷ is H;
X is H or CR¹⁸R¹⁹R²⁰, Y is a H or CR²¹R²²R²³, or X and Y, together with the carbon atoms they bond to, form a phenyl ring, a five- or six-membered cycloalkyl ring, or a five or six-membered heterocycloalkyl ring;
each of R²¹ to R²³ is H, halo, hydroxyl, thio, cyano, amino, nitro, carboxyl, C₁₋₆ alkyl, or C₁₋₆ alkoxy;
R²⁴ is H;
phenyl is unsubstituted or substituted with one or more of halo, hydroxyl, thio, cyano, amino, nitro, carboxyl, alkyl, alkoxy, and aryl; and
all other variables are defined above.

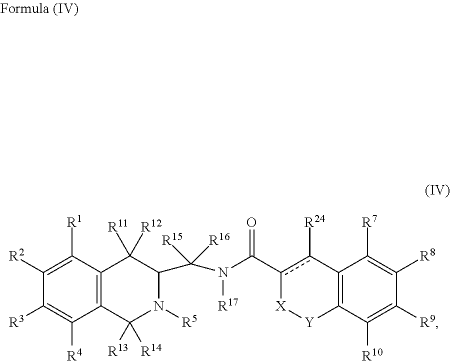

in which
each of R¹ to R⁴, R⁷ to R¹⁶, and R²⁴, independently, is H, halo, hydroxyl, cyano, amino, nitro, thio, carboxyl, C₁₋₆ alkyl, or C₁₋₆ alkoxy;
R⁵ is C₁₋₆ alkyl, C₃₋₈ cycloalkyl, or C₁₋₆ alkenyl;
R¹⁷ is H, C₁₋₆ alkyl, C₃₋₈ cycloalkyl, or C₁₋₆ alkenyl;
X is S, CR¹⁸R¹⁹, or NR¹⁸;
Y is a bond or CR²¹R²²;
each of R¹⁸, R¹⁹, R²¹, and R²², independently, is H, halo, hydroxyl, thio, cyano, amino, nitro, carboxyl, C₁₋₆ alkyl, or C₁₋₆ alkoxy;
each of amino, thio, alkyl, alkoxy, alkenyl, and cycloalkyl, independently, is unsubstituted or substituted with one or more of halo, hydroxyl, thio, cyano, amino, nitro, carboxyl, alkyl, alkoxy, and aryl, and
[symbol] is a single bond or double bond.

The compounds of Formula (V) is a substance of the compounds of Formula (II) above:

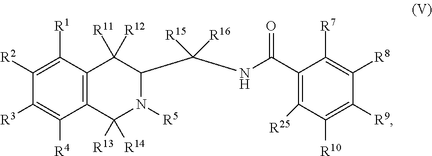

in which
each of R¹ to R⁴, independently, is H, halo, cyano, amino, nitro, thio, carboxyl, C₁₋₆ alkyl, or C₁₋₆ alkoxy;
R⁵ is methyl, C₁₋₆ alkoxyalkyl, or C₁₋₆ alkenyl;
each of R⁷ to R¹⁶ and R²⁵, independently, is H, halo, hydroxyl, cyano, amino, nitro, thio, carboxyl, C₁₋₆ alkyl, or C₁₋₆ alkoxy; and
alkoxyalkyl is unsubstituted or substituted with one or more of halo, hydroxyl, thio, cyano, amino, nitro, carboxyl, and aryl; and
all other variables are defined above.

Compounds of any one of Formula (I) to (V) above, when applicable, can have one of the following features, or any combinations thereof:
(i) each of R¹ to R⁴, independently, is H, halo, cyano, amino, nitro, thio, or C₁₋₆ alkyl;
(ii) each of R¹ to R⁴, independently, is H, halo, hydroxyl, cyano, amino, nitro, thio, carboxyl, C₁₋₆ alkyl, or C₁₋₆ alkoxy;
(iii) each of R¹ to R⁴, independently, is H, halo, cyano, amino, nitro, thio, carboxyl, C₁₋₆ alkyl, or C₁₋₆ alkoxy;
(iv) each of R¹ to R⁴, independently, is H, halo, cyano, amino, nitro, thio, or C₁₋₆ alkyl;
(v) each one of R¹ to R⁴, independently, is H, F, Br, Cl, methyl, or ethyl;
(vi) R¹ and R², independently, is H, F, Cl, Br, or methyl; R³ is H, F, Cl, or methyl; and R⁴ is H;
(vii) R⁵ is methyl, C₁₋₆ alkoxyalkyl, or C₁₋₆ alkenyl;
(viii) R⁵ is methyl, prop-2-en-yl, 3-methylbut-2-en-1-yl, or 2-methoxyethyl;
(ix) R⁵ is methyl;
(x) R⁶ is cyclopropyl, cyclobutyl, cyclopentyl, cyclohexyl, phenyl, xanthenyl, indolinyl, indanyl, indenyl, or tetralinyl, each of which is unsubstituted or substituted with one or more of F, Cl, methyl, ethyl, ethylthio, trifluoromethyl, and phenyl;
(xi) R⁶ is cyclopropyl, cyclobutyl, cyclohexyl, phenyl, indolinyl, indanyl, indenyl, or tetralinyl, each of which is unsubstituted or substituted with one or more of F, Cl, methyl, trifluoromethyl, ethylthio, and phenyl;
(xii) R⁶ is

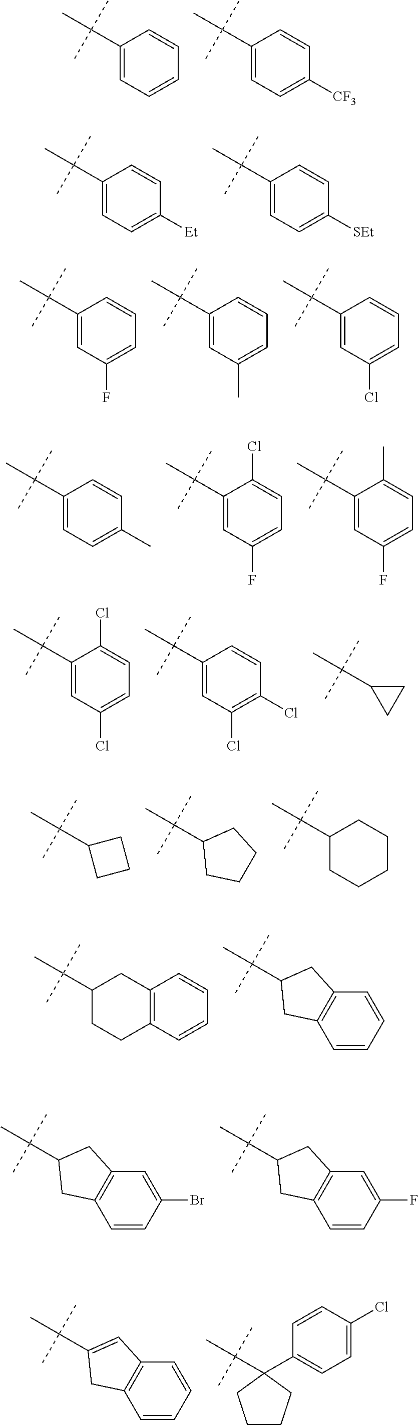

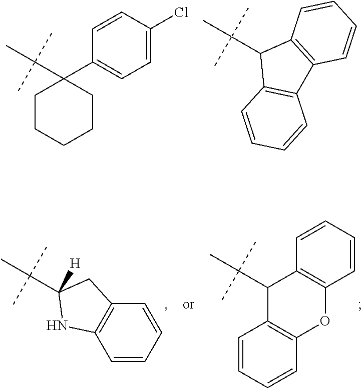

(xiii) R⁶ is

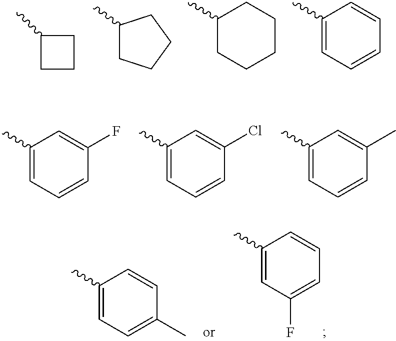

(xiv) each of R⁷ to R¹⁰, independently, is H, halo, hydroxyl, cyano, amino, nitro, thio, carboxyl, C₁₋₆ alkyl, or C₁₋₆ alkoxy;
(xv) each of R⁷ to R¹⁰, independently, is H, halo, cyano, amino, nitro, thio, carboxyl, or C₁₋₆ alkyl;
(xvi) each of R⁷ to R¹⁰, independently, is H, F, Cl, Br, CF₃, methyl, ethyl, or ethylthio;
(xvii) each of R⁷ to R¹⁰, independently, is H, F, Cl, CF₃, ethyl, or ethylthio;
(xviii) R⁷ is H; each of R⁸ and R¹⁰, independently, is H, F, Cl, or methyl; and R⁹ is H, Cl, CF₃, ethyl, or ethylthio;
(xix) each of R¹¹ to R¹⁶ is H;
(xx) each of m and n is 1 and R¹⁷ is H;
(xxi) each of m and n is 1 and R²⁴ is H;
(xxii) each of m and n is 0, X and R²⁴ is absent, and R⁶ is aryl or heteroaryl such as phenyl, benzothiophenyl, 9H-fluoren-9-yl, and 9H-xanthen-9-yl;
(xxiii) each of X and R²⁴ is H or X and R²⁴, together with the carbon atoms they bond to, form cyclopropylene;
R⁶ is

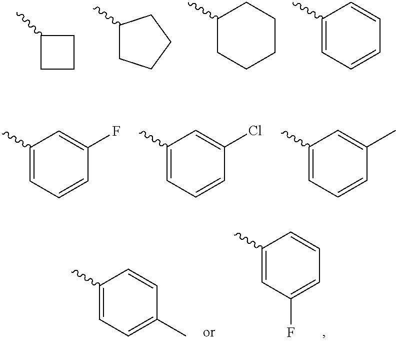

and [symbol] is a single bond or double bond;
(xxiv) X is H, NH, or CH₂;
(xxv) Y is H, a bond, or CH₂;
(xxvi) R²⁵ is H;
(xxvii) [symbol] is a single bond;
(xxviii) [symbol] is a double bond; and
(xxix) the stereoisomeric configuration of carbon-3 is R or S.

Exemplary compounds, Compounds 1-252, of this invention are shown in Table 1 below. Preferred compounds are Compounds 1, 5, 7, 12, 14, 15-17, 21, 26-28, 41-42, 61-65, 67-71, 74, 78, 80, 87, 97, 99, 102-105, and 111. Another set of preferred compounds are Compounds 19-21, 36, 41-43, 53, 67, 68, 70, 75, 102, 104, 105, 130, 131, and 163.

Further disclosed is a method of treating an opioid receptor-associated condition in a subject comprising administering to the subject in need thereof a pharmaceutically effective amount of a compound of Formula (I):

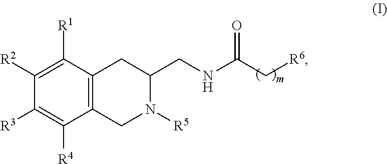

in which,
each of R¹-R⁴, independently, is H, halo, C₁₋₆ alkyl, C₁₋₆ alkoxy;
R⁵ is C₁₋₆ alkyl, C₃₋₈ cycloalkyl, C₁₋₆ alkenyl, or C₁₋₆ alkoxy, C₁₋₆ alkenylether;
R⁶ is C₁₋₆ alkyl, C₃₋₈ cycloalkyl, C₂₋₈ heterocycloalkyl, C₆₋₁₄ aryl, C₆₋₁₄ cycloalkylaryl, C₆₋₁₄ aralkyl, C₂₋₁₃ heteroaryl, or 6 to 16-membered heterocycloalkylaryl;
m is 0, 1 or 2;
each of alkyl, alkoxy, and alkenyl is unsubstituted or substituted with one or more of halo, alkylthio, and aryl; and
each of cycloalkyl, heterocycloalkyl, aryl, cycloalkylaryl, aralkyl, heteroaryl, and heterocycloalkylaryl is unsubstituted or substituted with one or more of halo, alkyl, alkoxy, alkylthio, and aryl.

In this method, the compound of Formula (I) can have one or more of the following features: (i) each of R¹-R⁴, independently, is H, F, Cl, Br, methyl, ethyl, or methoxy, preferably H, F, Cl, Br, or methyl; (ii) R⁵ is methyl, ethyl, propenyl, butenyl, pentenyl, or methoxyethyl, preferably is methyl; (iii) R⁶ is cyclopropyl, cyclobutyl, cyclopentyl, cyclohexyl, phenyl, xanthenyl, indolinyl, indanyl, indenyl, or tetralinyl, each of which is unsubstituted or substituted with one or more of F, Cl, Br, methyl, ethyl, ethylthio, trifluoromethyl, and phenyl, preferably cyclopropyl, cyclobutyl, cyclohexyl, phenyl, indolinyl, indanyl, indenyl, or tetralinyl, each of which is unsubstituted or substituted with one or more of F, Cl, Br, methyl, ethyl, trifluoromethyl, ethylthio, and phenyl. Examples include

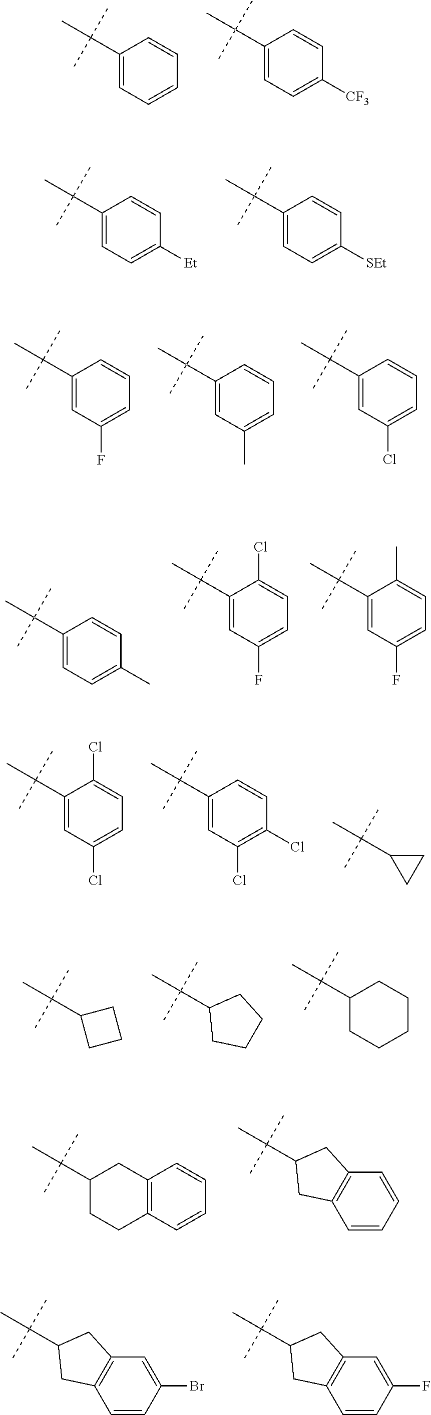

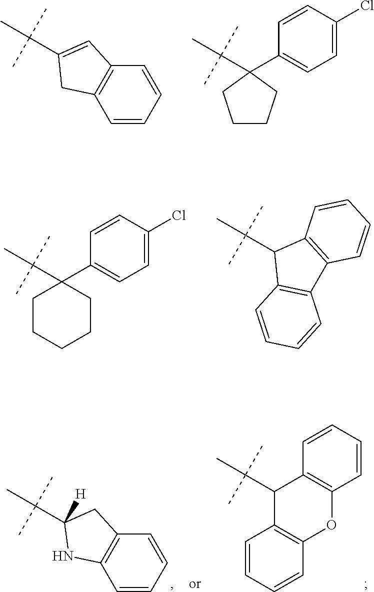

(iv) m is 0 or 2; (v) the stereoisomeric configuration of carbon-3 is R, (vi) the stereoisomeric configuration of carbon-3 is S, and (viii) the compound is a mixture of R- and S-stereoisomers at any ratio (e.g., 1:99 to 99:1, 1:9 to 9:1, 2:8 to 8:2, 3:7 to 7:3, and 1:1).

Further envisioned includes use of such a compound (e.g., a pharmaceutical composition containing one of the compounds described above) for treating an opioid receptor-associated condition or for the manufacture of a medicament for treating the condition.

Also provided are pharmaceutical compositions for treating an opioid receptor-associated condition in a subject. The pharmaceutical compositions each contain one of the compounds described above and a pharmaceutically acceptable carrier and any compound described above.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active compound. Examples of other carriers include colloidal silicon oxide, magnesium stearate, cellulose, sodium lauryl sulfate, and D&C Yellow #10.

The method of treating an opioid receptor-associated condition in a subject includes administering to the subject in need thereof a pharmaceutically effective amount of any compound or its pharmaceutical composition described supra.

Examples of the opioid receptor-associated condition include pain, immune disease, esophageal reflux, diarrhea, anxiety, and heroin addiction, especially pain such as cancer pain, post operative pain, low back pain, rheumatoid arthritis pain, osteoarthritis pain, neuropathic pain, and fibromyalgia pain.

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “halo” or “halogen” herein refers to a fluoro, chloro, bromo, or iodo radical. Examples include a fluoro radical (F), chloro radical (Cl), and a bromo radical (Br).

The term “cyano” refers to the “CN” radical. The term “nitro” refers to the “NO₂” radical.

The term “amino” refers to a radical derived from amine, which is unsubstituted or mono-/di-substituted with alkyl, aryl, cycloalkyl, heterocycloalkyl, or heteroaryl. The term “aminoalkyl” refers to NH₂-alkyl, i.e., an alkyl that is substituted with at least one amino group. The term “alkylamino” refers to alkyl-NH—. Examples of aminoalkyl include aminomethyl and 2-aminoethyl.

The term “alkyl” herein refers to a straight or branched hydrocarbon group, containing 1-20 (e.g., 1-10 and 1-6) carbon atoms. Examples include methyl (Me), ethyl (Et), n-propyl, i-propyl, n-butyl, i-butyl, and t-butyl. Alkyl includes haloalkyl. The term “haloalkyl” refers to alkyl substituted with one or more halogen (chloro, fluoro, bromo, or idodo) atoms. Examples include trifluoromethyl, bromomethyl, and 4,4,4-trifluorobutyl.

The term “alkylene” refers to a straight or branched hydrocarbon group, containing 1-20 carbon atoms (e.g., C₁₋₆ and C₁₋₃) and two monovalent radical centers or a bivalent radical center derived by the removal of two hydrogen atoms from one or more carbon atoms of a parent alkane. Exemplary alkylene groups include, but not limited to —CH₂—, —CH₂CH₂—, —CH(CH₃)—, —C(CH₃)₂—, —CH₂CH₂CH₂—, —CH(CH₃)CH₂—.

The term “alkenyl”, as employed herein as such or as part of another group, refers to an aliphatic hydrocarbon group having at least 2 carbon atoms and containing one or several double bonds. Representative examples include, but are not limited to, ethenyl, propenyl and butenyl.

The term “alkynyl”, as employed herein as such or as part of another group, refers to an aliphatic hydrocarbon group having at least 2 carbon atoms and containing one or several triple bonds. Representative examples include, but are not limited to, ethynyl, propynyl and butynyl.

The term “alkoxy” refers to an —O-alkyl group. Representative examples of C₁₋₇ alkoxy include, but are not limited to methoxy, ethoxy, propoxy, butoxy, isobutoxy, sec-butoxy and Ze/7-butoxy. Alkoxy also includes haloalkoxy, namely, alkoxy substituted with one or more halogens, e.g., —O—CH₂Cl, —O—CF₃ and —O—CHClCH₂Cl.

The term “thio” refers to —S—R^{b}, wherein R^{b} is H, alkyl, alkenyl, alkynyl, haloalkyl, heteroalkyl (bonded through a chain carbon), cycloalkyl, cycloalkylalkyl, aryl, aralkyl, heterocycloalkyl (bonded through a ring carbon), heterocycloalkylalkyl, heteroaryl (bonded through a ring carbon) or heteroarylalkyl. The term “alkylthio” refers to an-S-alkyl group. Representative examples of C₁₋₇ alkylthio include, but are not limited to methylthio, ethylthio, propylthio, butylthio, isobutylthio, sec-butylthio and Ze/7-butylthio. Alkylthio also includes haloalkylthio, namely, alkylthio substituted with one or more halogens, e.g., —S—CH₂Cl, —S—CF₃ and —S—CHClCH₂Cl.

The term “oxo”, as employed herein as such or as part of another group, refers to oxygen atom linked to another atom by a double bond (═O). The term “carbonyl” refers to —C(O)—. The term “carboxyl” refers to —COOH.

The term “cycloalkyl” refers to a saturated and partially unsaturated (i.e., nonaromatic) monocyclic, bicyclic, tricyclic, or tetracyclic hydrocarbon group having 3 to 12 carbons. Examples include cyclopropyl, cyclobutyl, cyclopentyl, cyclopentenyl, cyclohexyl, cyclohexenyl, cycloheptyl, cyclooctyl, indane, and indene. The term “heterocycloalkyl” refers to a nonaromatic 5-8 membered monocyclic, 8-12 membered bicyclic, or 11-14 membered tricyclic ring system having one or more heteroatoms (e.g., O, N, P, and S). Examples include piperazinyl, imidazolidinyl, azepanyl, pyrrolidinyl, dihydrothiadiazolyl, dioxanyl, morpholinyl, tetrahydropuranyl, and tetrahydrofuranyl.

The term “aralkyl” refers to alkyl substituted with an aryl group. Examples include benzyl and naphthylmethyl.

The term “aryl” refers to a 6-carbon monocyclic, 10-carbon bicyclic, 14-carbon tricyclic aromatic ring system. Examples of aryl groups include phenyl, naphthyl, and anthracenyl. The term “heteroaryl” refers to an aromatic 5-8 membered monocyclic, 8-12 membered bicyclic, or 11-14 membered tricyclic ring system having one or more heteroatoms (e.g., O, N, P, and S). Examples include triazolyl, oxazolyl, thiadiazolyl, tetrazolyl, pyrazolyl, pyridyl, furyl, imidazolyl, benzimidazolyl, pyrimidinyl, thienyl, quinolinyl, indolyl, indolinyl, thiazolyl, and benzothiazolyl. The term “cycloalkylaryl” refers to a cycloalkyl ring fused with aryl. The term “heterocycloalkylaryl” refers to a heterocycloalkyl ring fused with aryl.

Alkyl, alkenyl, amino, alkylthio, cycloalkyl, cycloalkylaryl, heterocycloalkyl, heterocycloalkylaryl, aralkyl, heteroaralkyl, alkoxy, aryl, and heteroaryl mentioned herein include both substituted and unsubstituted moieties. Possible substituents on amino, alkylthio, cycloalkyl, cycloalkylaryl, heterocycloalkyl, heterocycloalkylaryl, aralkyl, heteroaralkyl, alkoxy, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amido, amidino, guanidine, ureido, thioureido, cyano, nitro, nitroso, azido, acyl, thioacyl, acyloxy, carboxyl, and carboxylic ester. On the other hand, possible substituents on alkyl or alkenyl include all of the above-recited substituents except C₁-C₁₀ alkyl. Cycloalkyl, heterocycloalkyl, aryl, and heteroaryl can also be fused with each other.

In addition to the compounds of formula (I) described above, their pharmaceutically acceptable salts and solvates, where applicable, are also covered by this invention. A salt can be formed between an anion and a positively charged group (e.g., amino) on a compound. Examples of a suitable anion include chloride, bromide, iodide, sulfate, nitrate, phosphate, citrate, methanesulfonate, trifluoroacetate, acetate, malate, tosylate, tartrate, fumurate, succinate, glutamate, glucuronate, lactate, glutarate, and maleate. A salt can also be formed between a cation and a negatively charged group. Examples of a suitable cation include sodium ion, potassium ion, magnesium ion, calcium ion, and an ammonium cation such as tetramethylammonium ion. A salt further includes those containing quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine.

The term “compound”, when referring to a compound of this invention, also includes its salts, solvates, and prodrugs. The pharmaceutically acceptable salts include those listed in Handbook of Pharmaceutical Salts: Properties, Selection and Use, 2^{nd} Revised Edition, P. H. Stahl and C. G. Wermuth (Eds.), Wiley-VCH, New York, (2011). In addition to pharmaceutically acceptable salts, other salts are contemplated in the invention. They may serve as intermediates in the purification of compounds or in the preparation of other pharmaceutically acceptable salts, or are useful for identification, characterization or purification of compounds of the invention. A solvate refers to a complex formed between an active compound and a pharmaceutically acceptable solvent. Examples of a pharmaceutically acceptable solvent include water, ethanol, isopropanol, ethyl acetate, acetic acid, and ethanolamine. A prodrug refers to a compound that, after administration, is metabolized into a pharmaceutically active drug. Examples of a prodrug include esters and other pharmaceutically acceptable derivatives.

The compounds of the present invention may contain one or more non-aromatic double bonds or asymmetric centers. Each of them occurs as a racemate or a racemic mixture, a single R enantiomer, a single S enantiomer, an individual diastereomer, a diastereomeric mixture, a cis-isomer, or a trans-isomer. Compounds of such isomeric forms are within the scope of this invention. They can be present as a mixture or can be isolated using chiral synthesis or chiral separation technologies.

The term “treating” or “treatment” refers to administering one or more of the compounds to a subject with the purpose to confer a therapeutic effect, e.g., to slow, interrupt, arrest, control, or stop of the progression of an existing disorder and/or symptoms thereof, but does not necessarily indicate a total elimination of all symptoms. “An effective amount” refers to the amount of a compound that is required to confer the therapeutic effect. Effective doses will vary, as recognized by those skilled in the art, depending on the types of symptoms treated, route of administration, excipient usage, and the possibility of co-usage with other therapeutic treatment.

The term “subject” refers to an animal including human or non-human, such as a mammal. A human is a preferred subject.

A compound of this invention may be administered alone or in the form of a pharmaceutical composition with pharmaceutically acceptable carriers, diluents or excipients. Such pharmaceutical compositions and processes for making the same are known in the art (See, e.g., Remington: The Science and Practice of Pharmacy, A. Adejare, Editor, 23rd Edition., Academic Press, 2020).

To practice the method of the present invention, a composition or a kit containing one or more of the above-described compounds can be administered alone or co-administered with at least one other pharmacologically active substance simultaneously, concurrently, sequentially, successively, alternately, or separately. Simultaneous administration, also referring to as concomitant administration, includes administration at substantially the same time. Concurrent administration includes administering the active agents within the same general time period, for example on the same day(s) but not necessarily at the same time. Alternate administration includes administration of one agent during a time period, for example over the course of a few days or a week, followed by administration of the other agent(s) during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent(s) during a second and/or additional time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g., according to the agents used and the condition of the subject.

The compounds or pharmaceutical compositions may be administered (whether dependently or independently) by methods customary to the skilled person, e.g., by oral, enteral, parenteral, nasal, inhalation spray, implanted reservoir, vaginal, buccal, rectal, or topical routes of administration and may be formulated, alone or together, in suitable dosage unit formulations containing conventional non-toxic pharmaceutically acceptable carriers, excipients and/or vehicles appropriate for each route of administration.

The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Oral solid dosage forms can be prepared by spray dried techniques; hot melt extrusion strategy, micronization, and nano milling technologies.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having an active compound can also be administered in the form of suppositories for rectal administration.

The details of one or more embodiments of the invention are set forth in the description below. Other features, objects, and advantages of the invention will be apparent from the description and from the claims.

### DETAILED DESCRIPTION OF THE INVENTION

The compounds of this invention are MOR agonists useful for treating an opioid receptor-associated condition in a patient. In an exemplary in vitro screening method of this invention for identifying agonists of a MOR, cells that express a MOR are treated with a test compound in a cellular calcium fluorescent assay and intracellular calcium ([Ca²⁺]ᵢ) fluorescence intensity is then measured to determine whether the MOR is activated. A test compound is identified as an agonist of the MOR if the MOR is activated.

Two parameters, i.e., EC₅₀ and AUC, are typically used in such an assay to measure the degree of MOR activation exerted by the test compound. EC₅₀ herein refers to the concentration of a compound that induces a response halfway between the baseline and the maximum after a specified exposure time. AUC refers to the area under the response curve, an indication of the compound's capability of activating a MOR.

The compounds of this invention are also NOP agonists useful for treating an opioid receptor-associated condition in a patient. In an exemplary in vitro screening method of this invention for identifying agonists of a NOP, cells that express a NOP are treated with a test compound in a cellular CAMP assay and inhibition of cAMP production is then measured to determine whether the NOP is activated. A test compound is identified as an agonist of the NOP if the NOP is activated.

In an exemplary in vivo method of this invention for confirming that a test compound can produce in vivo antinociception. A test compound is injected into a mouse pain model (for example, subcutaneous but not limited), basal latencies are recorded before the treatment and test latencies are recorded at various specified times after the injection, and the (test latencies-basal latencies) values are calculated to determine whether an analgesic effect is exerted on the mouse. The test compound is confirmed to treat pain and the opioid receptor-associated condition upon observation of an antinociceptive effect.

Methods for synthesizing the compounds of Formula (I)-(V) are well known in the art. See, for example, R. Larock, Comprehensive Organic Transformations (2^{nd} Ed., VCH Publishers 1999); P. G. M. Wuts and T. W. Greene, Greene's Protective Groups in Organic Synthesis (4^{th} Ed., John Wiley and Sons 2007); L. Fieser and M. Fieser, Fieser and Fieser's Reagents for Organic Synthesis (John Wiley and Sons 1994); L. Paquette, ed., Encyclopedia of Reagents for Organic Synthesis (2^{nd} ed., John Wiley and Sons 2009); N. J. O'Reilly, W. S. Derwin, H. C. Lin “Optically Pure(S)-6,7-Dimethoxy-1,2,3,4-Tetrahydro-3-Isoquinolinecarboxylic Acid and Asymmetric Hydrogenation Studies Related to Its Preparation” Synthesis 7 (1990) 550-556; J. Liu, G. Cui, M. Zhao, C. Cui, J. Ju, S. Peng “Dual-acting agents that possess reversing resistance and anticancer activities: Design, synthesis, MES-SA/Dx5 cell assay, and SAR of Benzyl 1,2,3,5,11,11a-hexahydro-3,3-dimethyl-1-oxo-6H-imidazo[3′,4′:1,2]pyridin[3,4-b]indol-2-substituted acetates” Bioorganic & Medicinal Chemistry 15 (2007) 7773-7778; M. P. Chelopo, S. A. Pawar, M. K. Sokhela, T. Govender, H. G. Kruger, G. E. M. Maguire “Anticancer activity of ruthenium (II) arene complexes bearing 1,2,3,4-tetrahydroisoquinoline amino alcohol ligands” European Journal of Medicinal Chemistry 66 (2013) 407-414.

The compounds of Formulas (I)-(V) thus prepared can be initially screened using in vitro assays, e.g., the FLIPR® calcium and cAMP assays described in Example 253 below, for their potency in activating MOR and NOP in cells, respectively. They can be subsequently evaluated using in vivo assays, e.g., a tail-flick test assay described in Example 253 below. The selected compounds can be further tested to verify their efficacy in antinociception model.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

Shown in Table 1 below are the structures and names of 252 exemplary compounds of this invention. The methods for preparing these compounds, as well as the analytical data for the compounds thus prepared, are set forth in Examples 1-252 below. The procedures for testing these compounds are described in Examples 253 below.

All compounds were found to activate MOR to various degrees as indicated by their EC₅₀ and AUC values included Table 1. EC₅₀ values are presented in three ranges, i.e., +: more than 100 nM to 2.5 μM, ++: 1 to 100 nM, and +++: less than 1 nM. AUC values are presented in two ranges, i.e., ++: >10000 and +: 5000 to 10000.

TABLE 1

| | | | FLIPR Ca²⁺ EC₅₀ | FLIPR Ca²+ |
|---|---|---|---|---|
| No. | Name | Structure | (nM) | AUC |
| 1 | N-((2-Methyl-1,2,3,4- tetrahydroisoquinolin-3- yl)methyl)-4- (trifluoromethyl)benzamide | 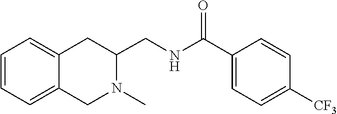 [SMILES: CN1Cc2ccccc2CC1CNC(=O)c1ccc(C(F)(F)F)cc1] | +++ | ++ |
| 2 | 4-Ethyl-N-((2-methyl- 1,2,3,4- tetrahydroisoquinolin-3- yl)methyl)benzamide | 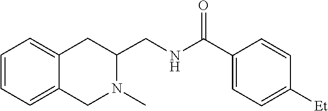 [SMILES: CCc1ccc(C(=O)NCC2Cc3ccccc3CN2C)cc1] | ++ | ++ |
| 3 | 4-(Ethylsulfanyl)-N-((2- methyl-1,2,3,4- tetrahydroisoquinolin-3- yl)methyl)benzamide | 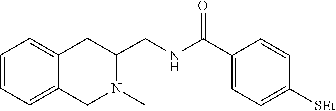 | ++ | ++ |
| 4 | 3-(3-Fluorophenyl)-N-((2- methyl-1,2,3,4- tetrahydroisoquinolin-3- yl)methyl)propanamide | 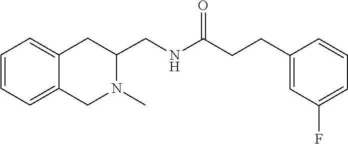 [SMILES: CN1Cc2ccccc2CC1CNC(=O)CCc1cccc(F)c1] | +++ | ++ |
| 5 | 3-Cyclopentyl-N-((2- methyl-1,2,3,4- tetrahydroisoquinolin-3- yl)methyl)propanamide | 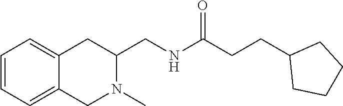 [SMILES: CN1Cc2ccccc2CC1CNC(=O)CCC1CCCC1] | +++ | ++ |
| 6 | 3-(4-Methylphenyl)-N-((2- methyl-1,2,3,4- tetrahydroisoquinolin-3- yl)methyl)propanamide | 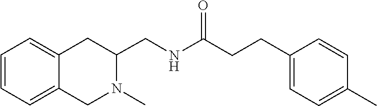 [SMILES: Cc1ccc(CCC(=O)NCC2Cc3ccccc3CN2C)cc1] | ++ | ++ |
| 7 | 3-(3-Methylphenyl)-N-((2- methyl-1,2,3,4- tetrahydroisoquinolin-3- yl)methyl)propanamide | 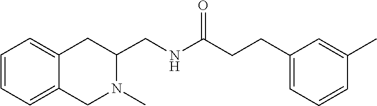 [SMILES: Cc1cccc(CCC(=O)NCC2Cc3ccccc3CN2C)c1] | +++ | ++ |
| 8 | 3-(3-Chlorophenyl)-N-((2- methyl-1,2,3,4- tetrahydroisoquinolin-3- yl)methyl)propanamide | 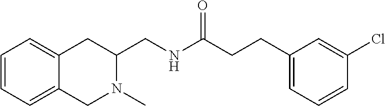 [SMILES: CN1Cc2ccccc2CC1CNC(=O)CCc1cccc(Cl)c1] | +++ | ++ |
| 9 | 2-Chloro-5-fluoro-N-((2- methyl-1,2,3,4- tetrahydroisoquinolin-3- yl)methyl)benzamide | 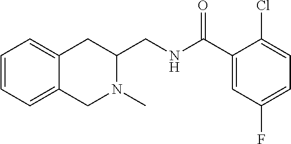 [SMILES: CN1Cc2ccccc2CC1CNC(=O)c1cc(F)ccc1Cl] | ++ | ++ |
| 10 | 5-Fluoro-2-methyl-N-((2- methyl-1,2,3,4- tetrahydroisoquinolin-3- yl)methyl)benzamide | 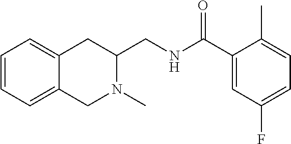 [SMILES: Cc1ccc(F)cc1C(=O)NCC1Cc2ccccc2CN1C] | ++ | ++ |
| 11 | 2,5-Dichloro-N-((2-methyl- 1,2,3,4- tetrahydroisoquinolin-3- yl)methyl)benzamide | 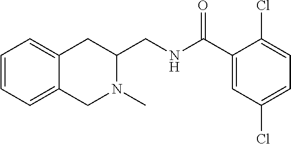 [SMILES: CN1Cc2ccccc2CC1CNC(=O)c1cc(Cl)ccc1Cl] | ++ | ++ |
| 12 | N-(((3R)-2-Methyl-1,2,3,4- tetrahydroisoquinolin-3- yl)methyl)-4- (trifluoromethyl)benzamide | 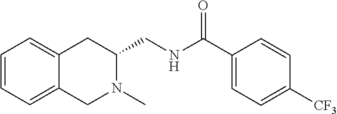 [SMILES: CN1Cc2ccccc2C[C@@H]1CNC(=O)c1ccc(C(F)(F)F)cc1] | +++ | ++ |
| 13 | 4-(Ethylsulfanyl)-N-(((3R)- 2-methyl-1,2,3,4- tetrahydroisoquinolin-3- yl)methyl)benzamide | 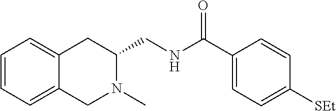 | +++ | ++ |
| 14 | 3,4-Dichloro-N-(((3R)-2- methyl-1,2,3,4- tetrahydroisoquinolin-3- yl)methyl)benzamide | 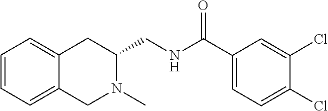 [SMILES: CN1Cc2ccccc2C[C@@H]1CNC(=O)c1ccc(Cl)c(Cl)c1] | +++ | ++ |
| 15 | 3-(3-Fluorophenyl)-N- (((3R)-2-methyl-1,2,3,4- tetrahydroisoquinolin-3- yl)methyl)propanamide | 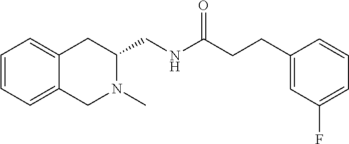 [SMILES: CN1Cc2ccccc2C[C@@H]1CNC(=O)CCc1cccc(F)c1] | +++ | ++ |
| 16 | N-(((3R)-2-Methyl-1,2,3,4- tetrahydroisoquinolin-3- yl)methyl)-3- phenylpropanamide | 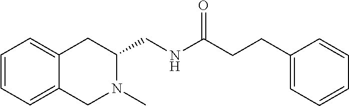 [SMILES: CN1Cc2ccccc2C[C@@H]1CNC(=O)CCc1ccccc1] | +++ | ++ |
| 17 | 3-Cyclohexyl-N-(((3R)-2- methyl-1,2,3,4- tetrahydroisoquinolin-3- yl)methyl)propanamide | 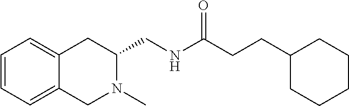 [SMILES: CN1Cc2ccccc2C[C@@H]1CNC(=O)CCC1CCCCC1] | +++ | ++ |
| 18 | 3-Cyclobutyl-N-(((3R)-2- methyl-1,2,3,4- tetrahydroisoquinolin-3- yl)methyl)propanamide | 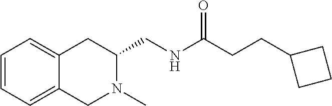 [SMILES: CN1Cc2ccccc2C[C@@H]1CNC(=O)CCC1CCC1] | +++ | ++ |
| 19 | (2R)-N-{[(3R)-2-Methyl- 1,2,3,4- tetrahydroisoquinolin-3- yl]methyl}-1,2,3,4- tetrahydronaphthalene-2- carboxamide | 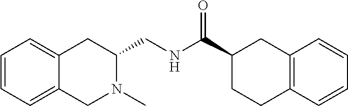 [SMILES: CN1Cc2ccccc2C[C@@H]1CNC(=O)[C@@H]1CCc2ccccc2C1] | +++ | ++ |
| 20 | (2S)-N-{[(3R)-2-Methyl- 1,2,3,4- tetrahydroisoquinolin-3- yl]methyl}-1,2,3,4- tetrahydronaphthalene-2- carboxamide | 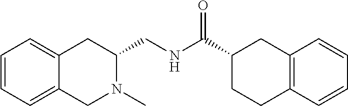 [SMILES: CN1Cc2ccccc2C[C@@H]1CNC(=O)[C@H]1CCc2ccccc2C1] | +++ | ++ |
| 21 | N-{[(3R)-2-Methyl-1,2,3,4- tetrahydroisoquinolin-3- yl]methyl}-2,3-dihydro-1H- indene-2-carboxamide | 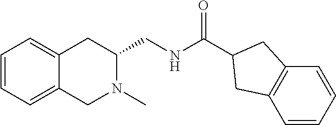 [SMILES: CN1Cc2ccccc2C[C@@H]1CNC(=O)C1Cc2ccccc2C1] | +++ | ++ |
| 22 | N-(((3S)-2-Methyl-1,2,3,4- tetrahydroisoquinolin-3- yl)methyl)-4- (trifluoromethyl)benzamide | 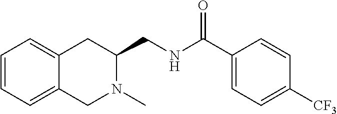 [SMILES: CN1Cc2ccccc2C[C@H]1CNC(=O)c1ccc(C(F)(F)F)cc1] | +++ | ++ |
| 23 | 4-Ethyl-N-(((3S)-2-methyl- 1,2,3,4- tetrahydroisoquinolin-3- yl)methyl)benzamide | 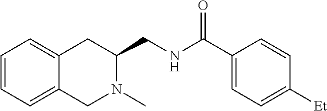 [SMILES: CCc1ccc(C(=O)NC[C@@H]2Cc3ccccc3CN2C)cc1] | ++ | ++ |
| 24 | 4-(Ethylsulfanyl)-N-(((3S)- 2-methyl-1,2,3,4- tetrahydroisoquinolin-3- yl)methyl)benzamide | 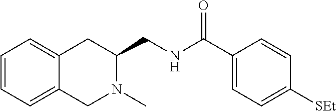 | ++ | ++ |
| 25 | 3,4-Dichloro-N-(((3S)-2- methyl-1,2,3,4- tetrahydroisoquinolin-3- yl)methyl)benzamide | 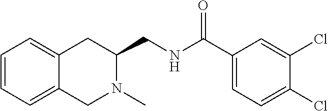 [SMILES: CN1Cc2ccccc2C[C@H]1CNC(=O)c1ccc(Cl)c(Cl)c1] | ++ | ++ |
| 26 | 3-(3-Fluorophenyl)-N- {[(3S)-2-methyl-1,2,3,4- tetrahydroisoquinolin-3- yl]methyl}propanamide | 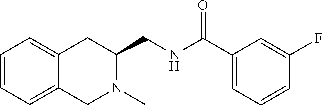 [SMILES: CN1Cc2ccccc2C[C@H]1CNC(=O)c1cccc(F)c1] | +++ | ++ |
| 27 | N-{[(3S)-2-Methyl-1,2,3,4- tetrahydroisoquinolin-3- yl]methyl}-3- phenylpropanamide | 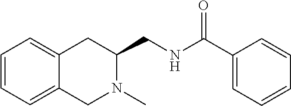 [SMILES: CN1Cc2ccccc2C[C@H]1CNC(=O)c1ccccc1] | +++ | ++ |
| 28 | 3-Cyclohexyl-N-{[(3S)-2- methyl-1,2,3,4- tetrahydroisoquinolin-3- yl]methyl}propanamide | 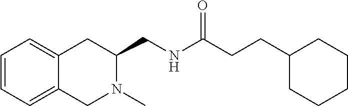 [SMILES: CN1Cc2ccccc2C[C@H]1CNC(=O)CCC1CCCCC1] | ++ | ++ |
| 29 | 3-Cyclobutyl-N-{[(3S)-2- methyl-1,2,3,4- tetrahydroisoquinolin-3- yl]methyl}propanamide | 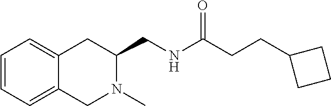 [SMILES: CN1Cc2ccccc2C[C@H]1CNC(=O)CCC1CCC1] | ++ | ++ |
| 30 | 3-Cyclopropyl-N-{[(3S)-2- methyl-1,2,3,4- tetrahydroisoquinolin-3- yl]methyl}propanamide | 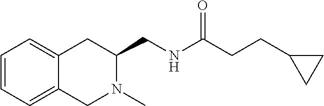 [SMILES: CN1Cc2ccccc2C[C@H]1CNC(=O)CCC1CC1] | ++ | ++ |
| 31 | N-(((3R)-2,5-Dimethyl- 1,2,3,4- tetrahydroisoquinolin-3- yl)methyl)-4- ethylbenzamide | 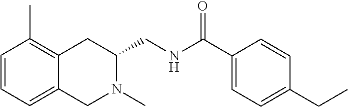 [SMILES: CCc1ccc(C(=O)NC[C@H]2Cc3c(C)cccc3CN2C)cc1] | ++ | ++ |
| 32 | 3,4-Dichloro-N-(((3R)-2,5- dimethyl-1,2,3,4- tetrahydroisoquinolin-3- yl)methyl)benzamide | 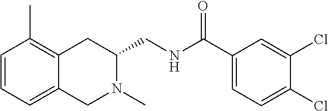 [SMILES: Cc1cccc2c1C[C@H](CNC(=O)c1ccc(Cl)c(Cl)c1)N(C)C2] | ++ | ++ |
| 33 | N-(((3R)-2,5-Dimethyl- 1,2,3,4- tetrahydroisoquinolin-3- yl)methyl)-3-(3- fluorophenyl)propanamide | 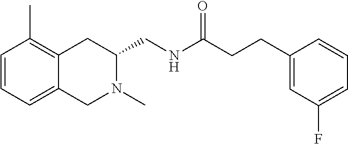 [SMILES: Cc1cccc2c1C[C@H](CNC(=O)CCc1cccc(F)c1)N(C)C2] | +++ | ++ |
| 34 | N-(((3R)-2,5-Dimethyl- 1,2,3,4- tetrahydroisoquinolin-3- yl)methyl)-3- phenylpropanamide | 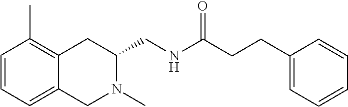 [SMILES: Cc1cccc2c1C[C@H](CNC(=O)CCc1ccccc1)N(C)C2] | +++ | ++ |
| 35 | 3-Cyclohexyl-N-(((3R)-2,5- dimethyl-1,2,3,4- tetrahydroisoquinolin-3- yl)methyl)propanamide | 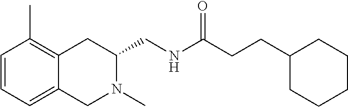 [SMILES: Cc1cccc2c1C[C@H](CNC(=O)CCC1CCCCC1)N(C)C2] | +++ | ++ |
| 36 | N-(((3R)-2,5-Dimethyl- 1,2,3,4- tetrahydroisoquinolin-3- yl)methyl)-2,3-dihydro-1H- indene-2-carboxamide | 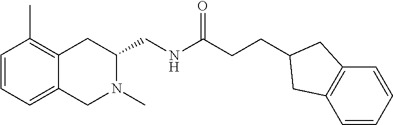 [SMILES: Cc1cccc2c1C[C@H](CNC(=O)CCC1Cc3ccccc3C1)N(C)C2] | +++ | ++ |
| 37 | N-(((3S)-2,5-Dimethyl- 1,2,3,4- tetrahydroisoquinolin-3- yl)methyl)-3-(3- fluorophenyl)propanamide | 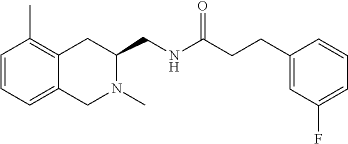 [SMILES: Cc1cccc2c1C[C@@H](CNC(=O)CCc1cccc(F)c1)N(C)C2] | ++ | ++ |
| 38 | N-(((3S)-2,5-Dimethyl- 1,2,3,4- tetrahydroisoquinolin-3- yl)methyl)-3- phenylpropanamide | 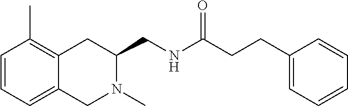 [SMILES: Cc1cccc2c1C[C@@H](CNC(=O)CCc1ccccc1)N(C)C2] | ++ | + |
| 39 | 3-Cyclohexyl-N-(((3S)-2,5- dimethyl-1,2,3,4- tetrahydroisoquinolin-3- yl)methyl)propanamide | 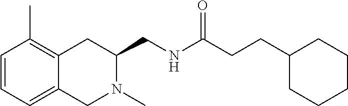 [SMILES: Cc1cccc2c1C[C@@H](CNC(=O)CCC1CCCCC1)N(C)C2] | ++ | + |
| 40 | N-[(5-Chloro-2-methyl- 1,2,3,4- tetrahydroisoquinolin-3- yl)methyl]-1,2,3,4- tetrahydronapthalene-2- carboxamide | 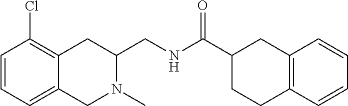 [SMILES: CN1Cc2cccc(Cl)c2CC1CNC(=O)C1CCc2ccccc2C1] | +++ | ++ |
| 41 | N-[(5-Chloro-2-methyl- 1,2,3,4- tetrahydroisoquinolin-3- yl)methyl]-2,3-hydroxy-1H- indene-2-carboxamide | 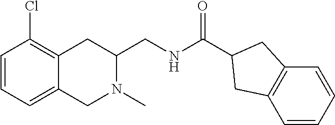 [SMILES: CN1Cc2cccc(Cl)c2CC1CNC(=O)C1Cc2ccccc2C1] | +++ | ++ |
| 42 | N-{[(3R)-5-Chloro-2- methyl-1,2,3,4- tetrahydroisoquinolin-3- yl]methyl}-1,2,3,4- tetrahydronaphthalene-2- carboxamide | 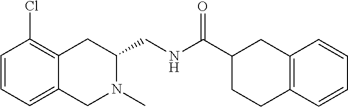 [SMILES: CN1Cc2cccc(Cl)c2C[C@@H]1CNC(=O)C1CCc2ccccc2C1] | +++ | ++ |
| 43 | N-{[(3R)-5-Chloro-2- methyl-1,2,3,4- tetrahydroisoquinolin-3- yl]methyl}-2,3-dihydro-1H- indene-2-carboxamide | 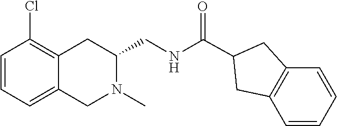 [SMILES: CN1Cc2cccc(Cl)c2C[C@@H]1CNC(=O)C1Cc2ccccc2C1] | ++ | ++ |
| 44 | N-((5-Bromo-2-methyl- 1,2,3,4- tetrahydroisoquinolin-3- yl)methyl)-3- phenylpropanamide | 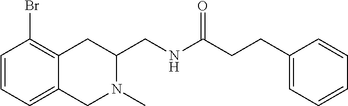 [SMILES: CN1Cc2cccc(Br)c2CC1CNC(=O)CCc1ccccc1] | ++ | ++ |
| 45 | N-((5-Bromo-2-methyl- 1,2,3,4- tetrahydroisoquinolin-3- yl)methyl)-3-(3- fluorophenyl)propanamide | 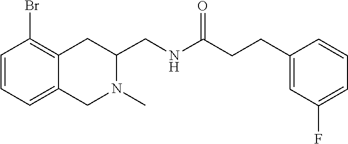 [SMILES: CN1Cc2cccc(Br)c2CC1CNC(=O)CCc1cccc(F)c1] | ++ | ++ |
| 46 | N-((5-Bromo-2-methyl- 1,2,3,4- tetrahydroisoquinolin-3- yl)methyl)-3- cyclohexylpropanamide | 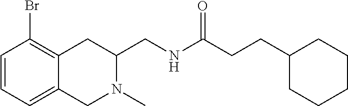 [SMILES: CN1Cc2cccc(Br)c2CC1CNC(=O)CCC1CCCCC1] | ++ | ++ |
| 47 | N-((5-Bromo-2-methyl- 1,2,3,4- tetrahydroisoquinolin-3- yl)methyl)-2,3-dihydro-1H- indene-2-carboxamide | 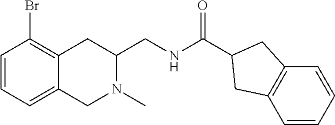 [SMILES: CN1Cc2cccc(Br)c2CC1CNC(=O)C1Cc2ccccc2C1] | +++ | ++ |
| 48 | N-((5-Bromo-2-methyl- 1,2,3,4- tetrahydroisoquinolin-3- yl)methyl)-3,4- dichlorobenzamide | 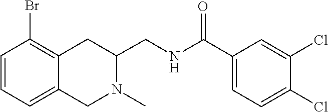 [SMILES: CN1Cc2cccc(Br)c2CC1CNC(=O)c1ccc(Cl)c(Cl)c1] | ++ | ++ |
| 49 | N-((5-Bromo-2-methyl- 1,2,3,4- tetrahydroisoquinolin-3- yl)methyl)-4- ethylbenzamide | 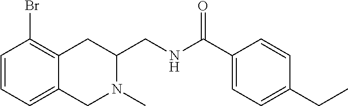 [SMILES: CCc1ccc(C(=O)NCC2Cc3c(Br)cccc3CN2C)cc1] | + | + |
| 50 | N-(((3R)-5-Bromo-2- methyl-1,2,3,4- tetrahydroisoquinolin-3- yl)methyl)-3- phenylpropanamide | 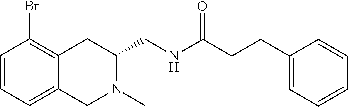 [SMILES: CN1Cc2cccc(Br)c2C[C@@H]1CNC(=O)CCc1ccccc1] | ++ | ++ |
| 51 | N-(((3R)-5-Bromo-2- methyl-1,2,3,4- tetrahydroisoquinolin-3- yl)methyl)-3-(3- fluorophenyl)propanamide | 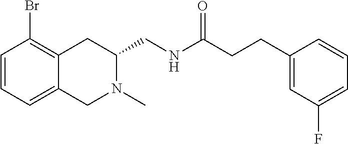 [SMILES: CN1Cc2cccc(Br)c2C[C@@H]1CNC(=O)CCc1cccc(F)c1] | ++ | ++ |
| 52 | N-(((3R)-5-Bromo-2- methyl-1,2,3,4- tetrahydroisoquinolin-3- yl)methyl)-3- cyclohexylpropanamide | 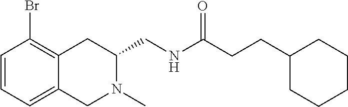 [SMILES: CN1Cc2cccc(Br)c2C[C@@H]1CNC(=O)CCC1CCCCC1] | ++ | ++ |
| 53 | N-(((3R)-5-Bromo-2- methyl-1,2,3,4- tetrahydroisoquinolin-3- yl)methyl)-2,3-dihydro-1H- indene-2-carboxamide | 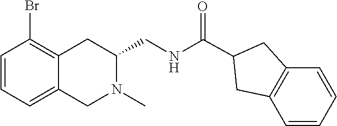 [SMILES: CN1Cc2cccc(Br)c2C[C@@H]1CNC(=O)C1Cc2ccccc2C1] | +++ | ++ |
| 54 | N-(((3R)-5-Bromo-2- methyl-1,2,3,4- tetrahydroisoquinolin-3- yl)methyl)-3,4- dichlorobenzamide | 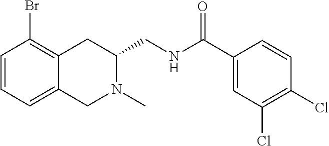 [SMILES: CN1Cc2cccc(Br)c2C[C@@H]1CNC(=O)c1ccc(Cl)c(Cl)c1] | ++ | ++ |
| 55 | N-(((3R)-5-Bromo-2- methyl-1,2,3,4- tetrahydroisoquinolin-3- yl)methyl)-4- ethylbenzamide | 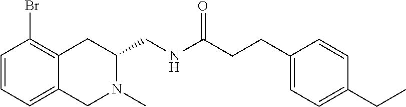 [SMILES: CCc1ccc(CCC(=O)NC[C@H]2Cc3c(Br)cccc3CN2C)cc1] | ++ | ++ |
| 56 | N-(((3S)-5-Bromo-2- methyl-1,2,3,4- tetrahydroisoquinolin-3- yl)methyl)-3- phenylpropanamide | 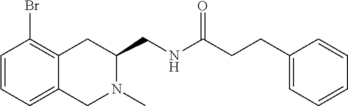 [SMILES: CN1Cc2cccc(Br)c2C[C@H]1CNC(=O)CCc1ccccc1] | + | ++ |
| 57 | N-(((3S)-5-Bromo-2- methyl-1,2,3,4- tetrahydroisoquinolin-3- yl)methyl)-3-(3- fluorophenyl)propanamide | 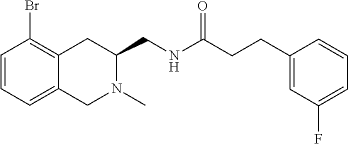 [SMILES: CN1Cc2cccc(Br)c2C[C@H]1CNC(=O)CCc1cccc(F)c1] | + | ++ |
| 58 | N-(((3S)-5-Bromo-2- methyl-1,2,3,4- tetrahydroisoquinolin-3- yl)methyl)-3- cyclohexylpropanamide | 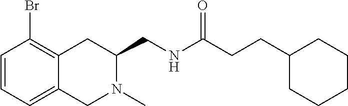 [SMILES: CN1Cc2cccc(Br)c2C[C@H]1CNC(=O)CCC1CCCCC1] | + | ++ |
| 59 | N-(((3S)-5-Bromo-2- methyl-1,2,3,4- tetrahydroisoquinolin-3- yl)methyl)-2,3-dihydro-1H- indene-2-carboxamide | 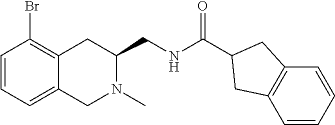 [SMILES: CN1Cc2cccc(Br)c2C[C@H]1CNC(=O)C1Cc2ccccc2C1] | ++ | ++ |
| 60 | N-(((3S)-5-Bromo-2- methyl-1,2,3,4- tetrahydroisoquinolin-3- yl)methyl)-3,4- dichlorobenzamide | 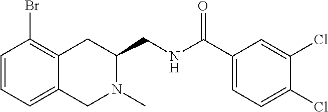 [SMILES: CN1Cc2cccc(Br)c2C[C@H]1CNC(=O)c1ccc(Cl)c(Cl)c1] | + | ++ |
| 61 | N-((2,6-Dimethyl-1,2,3,4- tetrahydroisoquinolin-3- yl)methyl)-4- (ethylsulfanyl)benzamide | 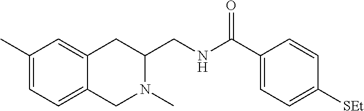 | +++ | ++ |
| 62 | 3,4-Dichloro-N-((2,6- dimethyl-1,2,3,4- tetrahydroisoquinolin-3- yl)methyl)benzamide | 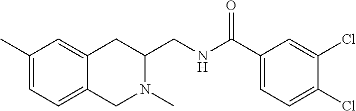 [SMILES: Cc1ccc2c(c1)CC(CNC(=O)c1ccc(Cl)c(Cl)c1)N(C)C2] | +++ | ++ |
| 63 | N-[(2,6-Dimethyl-1,2,3,4- tetrahydroisoquinolin-3- yl)methyl]-3- phenylpropanamide | 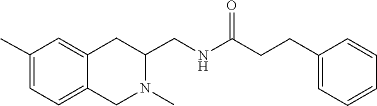 [SMILES: Cc1ccc2c(c1)CC(CNC(=O)CCc1ccccc1)N(C)C2] | +++ | ++ |
| 64 | 3-Cyclohexyl-N-[(2,6- dimethyl-1,2,3,4- tetrahydroisoquinolin-3- yl)methyl]propanamide | 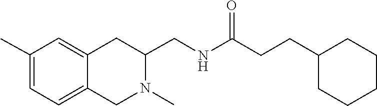 [SMILES: Cc1ccc2c(c1)CC(CNC(=O)CCC1CCCCC1)N(C)C2] | +++ | ++ |
| 65 | N-(((3R)-2,6-Dimethyl- 1,2,3,4- tetrahydroisoquinolin-3- yl)methyl)-4- (trifluoromethyl)benzamide | 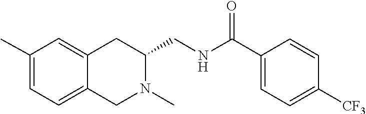 [SMILES: Cc1ccc2c(c1)C[C@H](CNC(=O)c1ccc(C(F)(F)F)cc1)N(C)C2] | +++ | ++ |
| 66 | N-(((3R)-2,6-Dimethyl- 1,2,3,4- tetrahydroisoquinolin-3- yl)methyl)-4- (ethylsulfanyl)benzamide | 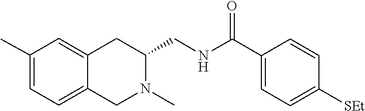 | +++ | ++ |
| 67 | N-(((3R)-2,6-Dimethyl- 1,2,3,4- tetrahydroisoquinolin-3- yl)methyl)-3-(3- fluorophenyl)propanamide | 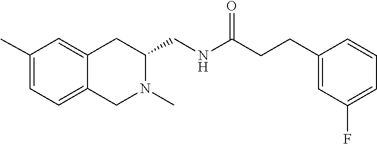 [SMILES: Cc1ccc2c(c1)C[C@H](CNC(=O)CCc1cccc(F)c1)N(C)C2] | +++ | ++ |
| 68 | N-(((3R)-2,6-Dimethyl- 1,2,3,4- tetrahydroisoquinolin-3- yl)methyl)-3- phenylpropanamide | 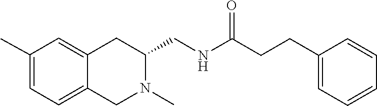 [SMILES: Cc1ccc2c(c1)C[C@H](CNC(=O)CCc1ccccc1)N(C)C2] | +++ | ++ |
| 69 | 3-Cyclohexyl-N-(((3R)-2,6- dimethyl-1,2,3,4- tetrahydroisoquinolin-3- yl)methyl)propanamide | 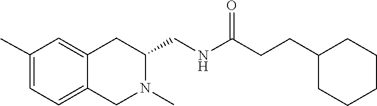 [SMILES: Cc1ccc2c(c1)C[C@H](CNC(=O)CCC1CCCCC1)N(C)C2] | +++ | ++ |
| 70 | N-{[(3R)-2,6-Dimethyl- 1,2,3,4- tetrahydroisoquinolin-3- yl]methyl}-2,3-dihydro-1H- indene-2-carboxamide | 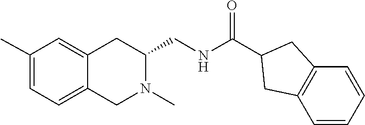 [SMILES: Cc1ccc2c(c1)C[C@H](CNC(=O)C1Cc3ccccc3C1)N(C)C2] | +++ | ++ |
| 71 | N-{[(3R)-2,6-Dimethyl- 1,2,3,4- tetrahydroisoquinolin-3- yl]methyl}-1,2,3,4- tetrahydronaphthalene-2- carboxamide | 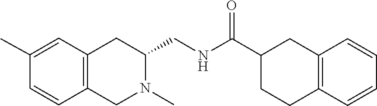 [SMILES: Cc1ccc2c(c1)C[C@H](CNC(=O)C1CCc3ccccc3C1)N(C)C2] | +++ | ++ |
| 72 | N-{[(3R)-2,6-Dimethyl- 1,2,3,4- tetrahydroisoquinolin-3- yl]methyl}-9H-fluorene-9- carboxamide | 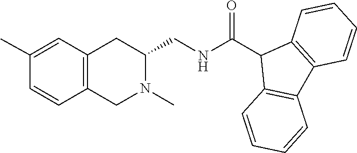 [SMILES: Cc1ccc2c(c1)C[C@H](CNC(=O)C1c3ccccc3-c3ccccc31)N(C)C2] | + | ++ |
| 73 | 5-Bromo-N-{[(3R)-2,6- dimethyl-1,2,3,4- tetrahydroisoquinolin-3- yl]methyl}-2,3-dihydro-1H- indene-2-carboxamide | 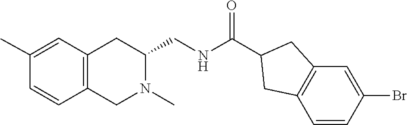 [SMILES: Cc1ccc2c(c1)C[C@H](CNC(=O)C1Cc3ccc(Br)cc3C1)N(C)C2] | ++ | ++ |
| 74 | N-{[(3R)-2,6-Dimethyl- 1,2,3,4- tetrahydroisoquinolin-3- yl]methyl}-5-fluoro-2,3- dihydro-1H-indene-2- carboxamide | 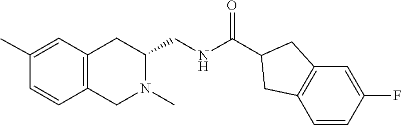 [SMILES: Cc1ccc2c(c1)C[C@H](CNC(=O)C1Cc3ccc(F)cc3C1)N(C)C2] | ++ | ++ |
| 75 | N-{[(3R)-2,6-Dimethyl- 1,2,3,4- tetrahydroisoquinolin-3- yl]methyl}-1H-indene-2- carboxamide | 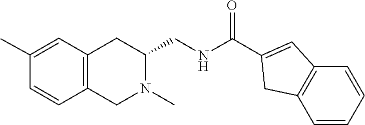 [SMILES: Cc1ccc2c(c1)C[C@H](CNC(=O)C1=Cc3ccccc3C1)N(C)C2] | +++ | ++ |
| 76 | 1-(4-Chlorophenyl)-N- {[(3R)-2,6-dimethyl- 1,2,3,4- tetrahydroisoquinolin-3- yl]methyl}cyclopentane- carboxamide | 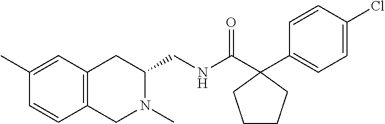 [SMILES: Cc1ccc2c(c1)C[C@H](CNC(=O)C1(c3ccc(Cl)cc3)CCCC1)N(C)C2] | + | ++ |
| 77 | 1-(4-Chlorophenyl)-N- {[(3R)-2,6-dimethyl- 1,2,3,4- tetrahydroisoquinolin-3- yl]methyl}cyclohexane- carboxamide | 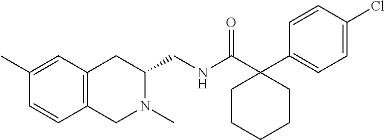 [SMILES: Cc1ccc2c(c1)C[C@H](CNC(=O)C1(c3ccc(Cl)cc3)CCCCC1)N(C)C2] | + | ++ |
| 78 | (2S)-N-{[(3R)-2,6- Dimethyl-1,2,3,4- tetrahydroisoquinolin-3- yl]methyl}-2,3-dihydro-1H- indole-2-carboxamide | 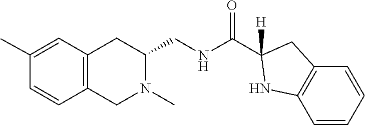 [SMILES: Cc1ccc2c(c1)C[C@H](CNC(=O)[C@@H]1Cc3ccccc3N1)N(C)C2] | ++ | ++ |
| 79 | N-{[(3R)-2,6-Dimethyl- 1,2,3,4- tetrahydroisoquinolin-3- yl]methyl}-9H-xanthene-9- carboxamide | 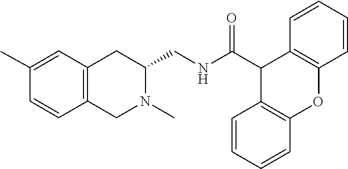 [SMILES: Cc1ccc2c(c1)C[C@H](CNC(=O)C1c3ccccc3Oc3ccccc31)N(C)C2] | ++ | ++ |
| 80 | trans-N-{[(3R)-2,6- Dimethyl-1,2,3,4- tetrahydroisoquinolin-3- yl]methyl}-2- phenylcyclopropanecarboxamide | 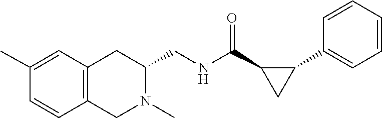 [SMILES: Cc1ccc2c(c1)C[C@H](CNC(=O)[C@@H]1C[C@H]1c1ccccc1)N(C)C2] | ++ | ++ |
| 81 | 3-(3-Fluorophenyl)-N- (((3R)-6-methoxy-2-methyl- 1,2,3,4- tetrahydroisoquinolin-3- yl)methyl)propanamide | 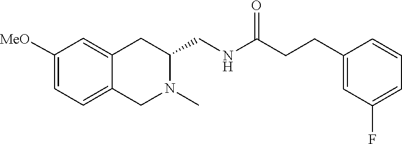 [SMILES: COc1ccc2c(c1)C[C@H](CNC(=O)CCc1cccc(F)c1)N(C)C2] | +++ | ++ |
| 82 | N-(((3R)-6-Methoxy-2- methyl-1,2,3,4- tetrahydroisoquinolin-3- yl)methyl)-3- phenylpropanamide | 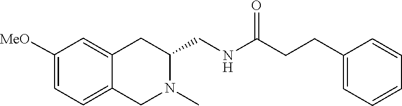 [SMILES: COc1ccc2c(c1)C[C@H](CNC(=O)CCc1ccccc1)N(C)C2] | ++ | ++ |
| 83 | 3-Cyclohexyl-N-(((3R)-6- methoxy-2-methyl-1,2,3,4- tetrahydroisoquinolin-3- yl)methyl)propanamide | 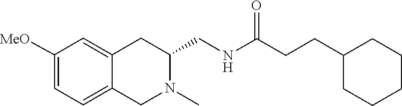 [SMILES: COc1ccc2c(c1)C[C@H](CNC(=O)CCC1CCCCC1)N(C)C2] | ++ | ++ |
| 84 | N-(((3R)-6-Ethyl-2-methyl- 1,2,3,4- tetrahydroisoquinolin-3- yl)methyl)-3- phenylpropanamide | 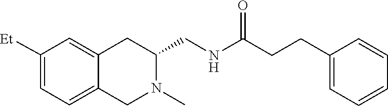 [SMILES: CCc1ccc2c(c1)C[C@H](CNC(=O)CCc1ccccc1)N(C)C2] | ++ | ++ |
| 85 | 3-Cyclohexyl-N-(((3R)-6- ethyl-2-methyl-1,2,3,4- tetrahydroisoquinolin-3- yl)methyl)propanamide | 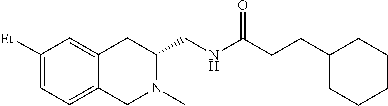 [SMILES: CCc1ccc2c(c1)C[C@H](CNC(=O)CCC1CCCCC1)N(C)C2] | ++ | ++ |
| 86 | 4-Ethyl-N-[(6-fluoro-2- methyl-1,2,3,4- tetrahydroisoquinolin-3- yl)methyl]benzamide | 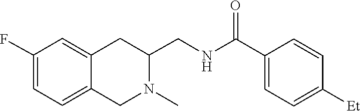 [SMILES: CCc1ccc(C(=O)NCC2Cc3cc(F)ccc3CN2C)cc1] | + | ++ |
| 87 | 3,4-Dichloro-N-[(6-fluoro- 2-methyl-1,2,3,4- tetrahydroisoquinolin-3- yl)methyl]benzamide | 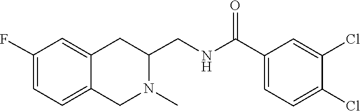 [SMILES: CN1Cc2ccc(F)cc2CC1CNC(=O)c1ccc(Cl)c(Cl)c1] | ++ | ++ |
| 88 | N-[(6-Fluoro-2-methyl- 1,2,3,4- tetrahydroisoquinolin-3- yl)methyl]-4- (trifluoromethyl)benzamide | 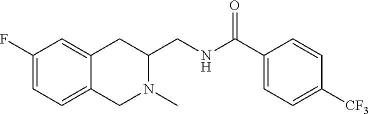 [SMILES: CN1Cc2ccc(F)cc2CC1CNC(=O)c1ccc(C(F)(F)F)cc1] | +++ | ++ |
| 89 | N-[(6-Fluoro-2-methyl- 1,2,3,4- tetrahydroisoquinolin-3- yl)methyl]-3-(3- fluorophenyl)propanamide | 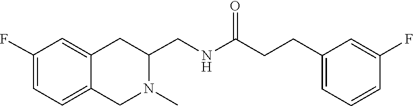 [SMILES: CN1Cc2ccc(F)cc2CC1CNC(=O)CCc1cccc(F)c1] | +++ | ++ |
| 90 | N-{[(3R)-6-Fluoro-2- methyl-1,2,3,4- tetrahydroisoquinolin-3- yl]methyl}-2,3-dihydro-1H- indene-2-carboxamide | 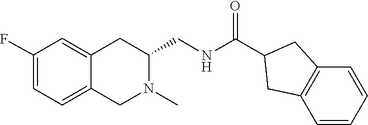 [SMILES: CN1Cc2ccc(F)cc2C[C@@H]1CNC(=O)C1Cc2ccccc2C1] | +++ | ++ |
| 91 | N-{[(3R)-6-Fluoro-2- methyl-1,2,3,4- tetrahydroisoquinolin-3- yl]methyl}-1,2,3,4- tetrahydronaphthalene-2- carboxamide | 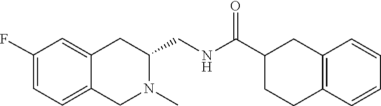 [SMILES: CN1Cc2ccc(F)cc2C[C@@H]1CNC(=O)C1CCc2ccccc2C1] | +++ | ++ |
| 92 | trans-N-{[(3R)-6-Fluoro-2- methyl-1,2,3,4- tetrahydroisoquinolin-3- yl]methyl}-2- phenylcyclopropanecarboxamide | 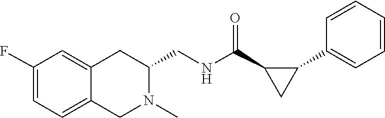 [SMILES: CN1Cc2ccc(F)cc2C[C@@H]1CNC(=O)[C@@H]1C[C@H]1c1ccccc1] | ++ | ++ |
| 93 | N-((6-Chloro-2-methyl- 1,2,3,4- tetrahydroisoquinolin-3- yl)methyl)-4- ethylbenzamide | 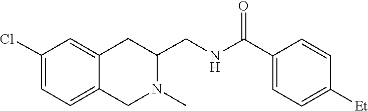 [SMILES: CCc1ccc(C(=O)NCC2Cc3cc(Cl)ccc3CN2C)cc1] | ++ | ++ |
| 94 | N-((6-Chloro-2-methyl- 1,2,3,4- tetrahydroisoquinolin-3- yl)methyl)-4- (ethylsulfanyl)benzamide | 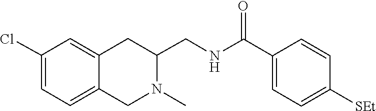 | ++ | ++ |
| 95 | 3,4-Dichloro-N-((6-chloro- 2-methyl-1,2,3,4- tetrahydroisoquinolin-3- yl)methyl)benzamide | 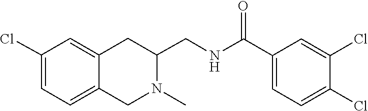 [SMILES: CN1Cc2ccc(Cl)cc2CC1CNC(=O)c1ccc(Cl)c(Cl)c1] | ++ | ++ |
| 96 | N-((6-Chloro-2-methyl- 1,2,3,4- tetrahydroisoquinolin-3- yl)methyl)-4- (trifluoromethyl)benzamide | 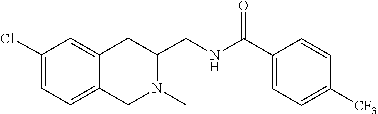 [SMILES: CN1Cc2ccc(Cl)cc2CC1CNC(=O)c1ccc(C(F)(F)F)cc1] | +++ | ++ |
| 97 | N-[(6-Chloro-2-methyl- 1,2,3,4- tetrahydroisoquinolin-3- yl)methyl]-3-(3- fluorophenyl)propanamide | 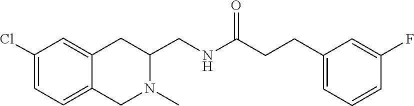 [SMILES: CN1Cc2ccc(Cl)cc2CC1CNC(=O)CCc1cccc(F)c1] | +++ | ++ |
| 98 | N-(((3R)-6-Chloro-2- methyl-1,2,3,4- tetrahydroisoquinolin-3- yl)methyl)-4- (trifluoromethyl)benzamide | 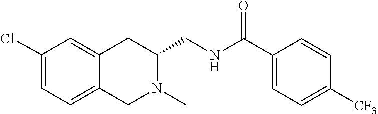 [SMILES: CN1Cc2ccc(Cl)cc2C[C@@H]1CNC(=O)c1ccc(C(F)(F)F)cc1] | +++ | ++ |
| 99 | N-(((3R)-6-Chloro-2- methyl-1,2,3,4- tetrahydroisoquinolin-3- yl)methyl)-3-(3- fluorophenyl)propanamide | 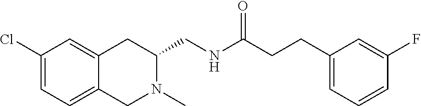 [SMILES: CN1Cc2ccc(Cl)cc2C[C@@H]1CNC(=O)CCc1cccc(F)c1] | +++ | ++ |
| 100 | N-(((3R)-6-Chloro-2- methyl-1,2,3,4- tetrahydroisoquinolin-3- yl)methyl)-3- cyclohexylpropanamide | 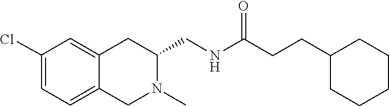 [SMILES: CN1Cc2ccc(Cl)cc2C[C@@H]1CNC(=O)CCC1CCCCC1] | +++ | ++ |
| 101 | N-(((3R)-6-Chloro-2- methyl-1,2,3,4- tetrahydroisoquinolin-3- yl)methyl)-3- phenylpropanamide | 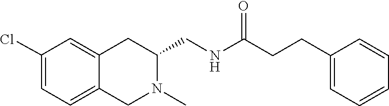 [SMILES: CN1Cc2ccc(Cl)cc2C[C@@H]1CNC(=O)CCc1ccccc1] | ++ | ++ |
| 102 | N-{[(3R)-6-Chloro-2- methyl-1,2,3,4- tetrahydroisoquinolin-3- yl]methyl}-1,2,3,4- tetrahydronaphthalene-2- carboxamide | 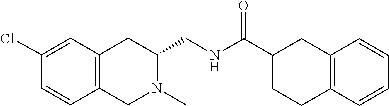 [SMILES: CN1Cc2ccc(Cl)cc2C[C@@H]1CNC(=O)C1CCc2ccccc2C1] | +++ | ++ |
| 103 | trans-N-{[(3R)-6-Chloro-2- methyl-1,2,3,4- tetrahydroisoquinolin-3- yl]methyl}-2- phenylcyclopropanecarboxamide | 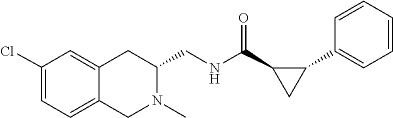 [SMILES: CN1Cc2ccc(Cl)cc2C[C@@H]1CNC(=O)[C@@H]1C[C@H]1c1ccccc1] | +++ | ++ |
| 104 | N-{[(3R)-6-Chloro-2- methyl-1,2,3,4- tetrahydroisoquinolin-3- yl]methyl}-2,3-dihydro-1H- indene-2-carboxamide | 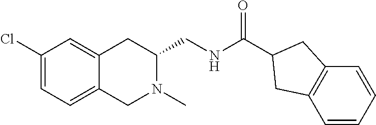 [SMILES: CN1Cc2ccc(Cl)cc2C[C@@H]1CNC(=O)C1Cc2ccccc2C1] | +++ | ++ |
| 105 | N-{[(3R)-6-Bromo-2- methyl-1,2,3,4- tetrahydroisoquinolin-3- yl]methyl}-2,3-dihydro-1H- indene-2-carboxamide | 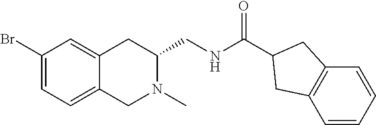 [SMILES: CN1Cc2ccc(Br)cc2C[C@@H]1CNC(=O)C1Cc2ccccc2C1] | +++ | ++ |
| 106 | N-((2,7-Dimethyl-1,2,3,4- tetrahydroisoquinolin-3- yl)methyl)-4- (trifluoromethyl)benzamide | 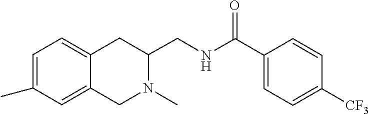 [SMILES: Cc1ccc2c(c1)CN(C)C(CNC(=O)c1ccc(C(F)(F)F)cc1)C2] | ++ | ++ |
| 107 | N-((2,7-Dimethyl-1,2,3,4- tetrahydroisoquinolin-3- yl)methyl)-4- ethylbenzamide | 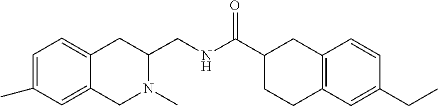 [SMILES: CCc1ccc2c(c1)CCC(C(=O)NCC1Cc3ccc(C)cc3CN1C)C2] | ++ | ++ |
| 108 | N-((2,7-Dimethyl-1,2,3,4- tetrahydroisoquinolin-3- yl)methyl)-4- (ethylsulfanyl)benzamide | 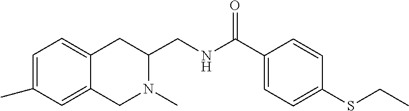 [SMILES: CCSc1ccc(C(=O)NCC2Cc3ccc(C)cc3CN2C)cc1] | ++ | ++ |
| 109 | 3,4-Dichloro-N-((2,7- dimethyl-1,2,3,4- tetrahydroisoquinolin-3- yl)methyl)benzamide | 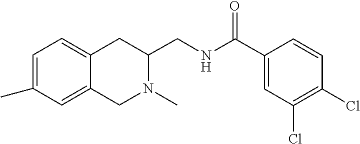 [SMILES: Cc1ccc2c(c1)CN(C)C(CNC(=O)c1ccc(Cl)c(Cl)c1)C2] | ++ | ++ |
| 110 | 4-(Ethylsulfanyl)-N-[(7- fluoro-2-methyl-1,2,3,4- tetrahydroisoquinolin-3- yl)methyl]benzamide | 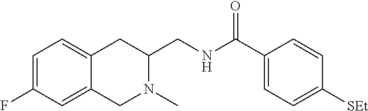 | ++ | ++ |
| 111 | N-[(7-Fluoro-2-methyl- 1,2,3,4- tetrahydroisoquinolin-3- yl)methyl]-4- (trifluoromethyl)benzamide | 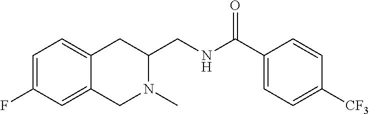 [SMILES: CN1Cc2cc(F)ccc2CC1CNC(=O)c1ccc(C(F)(F)F)cc1] | ++ | ++ |
| 112 | 4-Ethyl-N-[(7-fluoro-2- methyl-1,2,3,4- tetrahydroisoquinolin-3- yl)methyl]benzamide | 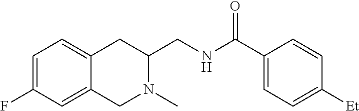 [SMILES: CCc1ccc(C(=O)NCC2Cc3ccc(F)cc3CN2C)cc1] | ++ | ++ |
| 113 | 3,4-Dichloro-N-[(7-fluoro- 2-methyl-1,2,3,4- tetrahydroisoquinolin-3- yl)methyl]benzamide | 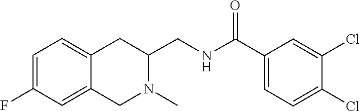 [SMILES: CN1Cc2cc(F)ccc2CC1CNC(=O)c1ccc(Cl)c(Cl)c1] | ++ | ++ |
| 114 | N-((7-Chloro-2-methyl- 1,2,3,4- tetrahydroisoquinolin-3- yl)methyl)-4- (trifluoromethyl)benzamide | 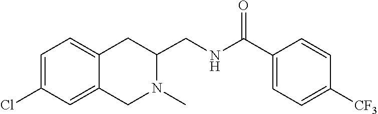 [SMILES: CN1Cc2cc(Cl)ccc2CC1CNC(=O)c1ccc(C(F)(F)F)cc1] | ++ | ++ |
| 115 | 3,4-Dichloro-N-((7-chloro- 2-methyl-1,2,3,4- tetrahydroisoquinolin-3- yl)methyl)benzamide | 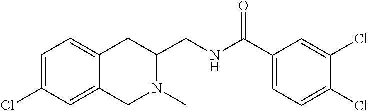 [SMILES: CN1Cc2cc(Cl)ccc2CC1CNC(=O)c1ccc(Cl)c(Cl)c1] | ++ | ++ |
| 116 | N-((2-(Prop-2-en-1-yl)- 1,2,3,4- tetrahydroisoquinolin-3- yl)methyl)-4- (trifluoromethyl)benzamide | 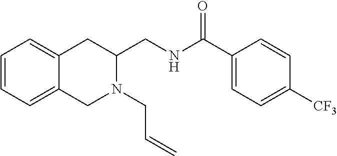 [SMILES: C=CCN1Cc2ccccc2CC1CNC(=O)c1ccc(C(F)(F)F)cc1] | + | ++ |
| 117 | 4-(Ethylsulfanyl)-N-((2- (prop-2-en-1-yl)-1,2,3,4- tetrahydroisoquinolin-3- yl)methyl)benzamide | 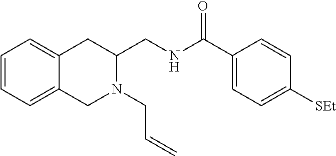 | + | ++ |
| 118 | 3,4-Dichloro-N-((2-(prop-2- en-1-yl)-1,2,3,4- tetrahydroisoquinolin-3- yl)methyl)benzamide | 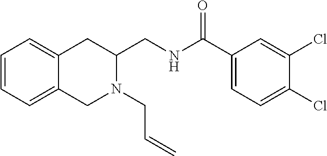 [SMILES: C=CCN1Cc2ccccc2CC1CNC(=O)c1ccc(Cl)c(Cl)c1] | ++ | ++ |
| 119 | N-((2-(3-Methylbut-2-en-1- yl)-1,2,3,4- tetrahydroisoquinolin-3- yl)methyl)-4- (trifluoromethyl)benzamide | 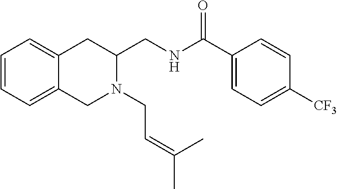 [SMILES: CC(C)=CCN1Cc2ccccc2CC1CNC(=O)c1ccc(C(F)(F)F)cc1] | ++ | ++ |
| 120 | 4-Ethyl-N-((2-(3-methylbut- 2-en-1-yl)-1,2,3,4- tetrahydroisoquinolin-3- yl)methyl)benzamide | 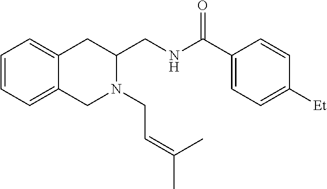 [SMILES: CCc1ccc(C(=O)NCC2Cc3ccccc3CN2CC=C(C)C)cc1] | + | ++ |
| 121 | 4-(Ethylsulfanyl)-N-((2-(3- methylbut-2-en-1-yl)- 1,2,3,4- tetrahydroisoquinolin-3- yl)methyl)benzamide | 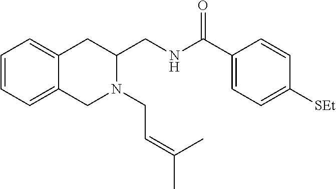 | ++ | ++ |
| 122 | 3,4-Dichloro-N-((2-(3- methylbut-2-en-1-yl)- 1,2,3,4- tetrahydroisoquinolin-3- yl)methyl)benzamide | 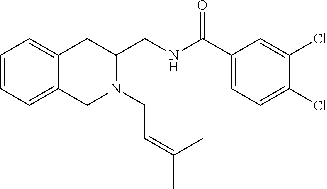 [SMILES: CC(C)=CCN1Cc2ccccc2CC1CNC(=O)c1ccc(Cl)c(Cl)c1] | + | ++ |
| 123 | N-{[2-(2-Methoxyethyl)- 1,2,3,4- tetrahydroisoquinolin-3- yl]methyl}-4- (trifluoromethyl)benzamide | 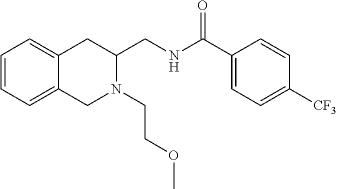 [SMILES: COCCN1Cc2ccccc2CC1CNC(=O)c1ccc(C(F)(F)F)cc1] | ++ | ++ |
| 124 | 4-(Ethylsulfanyl)-N-{[2-(2- methoxyethyl)-1,2,3,4- tetrahydroisoquinolin-3- yl]methyl}benzamide | 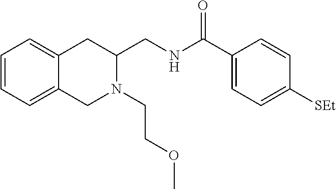 | ++ | ++ |
| 125 | 3,4-Dichloro-N-{[2-(2- methoxyethyl)-1,2,3,4- tetrahydroisoquinolin-3- yl]methyl}benzamide | 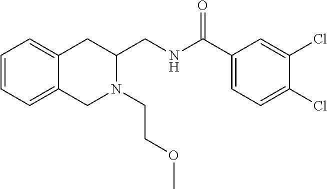 [SMILES: COCCN1Cc2ccccc2CC1CNC(=O)c1ccc(Cl)c(Cl)c1] | + | ++ |
| 126 | 3,4-Dichloro-N-((2-methyl- 1,2,3,4- tetrahydroisoquinolin-3- yl)methyl)benzamide | 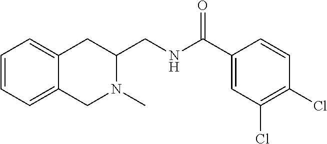 [SMILES: CN1Cc2ccccc2CC1CNC(=O)c1ccc(Cl)c(Cl)c1] | ++ | ++ |
| 127 | N-((2-Methyl-1,2,3,4- tetrahydroisoquinolin-3- yl)methyl)-3- phenylpropanamide | 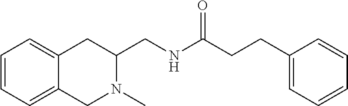 [SMILES: CN1Cc2ccccc2CC1CNC(=O)CCc1ccccc1] | +++ | ++ |
| 128 | 3-Cyclohexyl-N-((2-methyl- 1,2,3,4- tetrahydroisoquinolin-3- yl)methyl)propenamide | 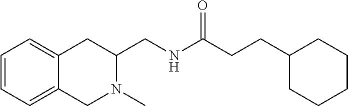 [SMILES: CN1Cc2ccccc2CC1CNC(=O)CCC1CCCCC1] | +++ | ++ |
| 129 | 3-Cyclobutyl-N-((2-methyl- 1,2,3,4- tetrahydroisoquinolin-3- yl)methyl)propanamide | 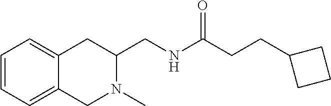 [SMILES: CN1Cc2ccccc2CC1CNC(=O)CCC1CCC1] | ++ | ++ |
| 130 | (2R)-N-((2-Methyl-1,2,3,4- tetrahydroisoquinolin-3- yl)methyl)-1,2,3,4- tetrahydronaphthalene-2- carboxamide | 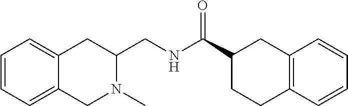 | +++ | ++ |
| 131 | (2S)-N-((2-Methyl-1,2,3,4- tetrahydroisoquinolin-3- yl)methyl)-1,2,3,4- tetrahydronaphthalene-2- carboxamide | 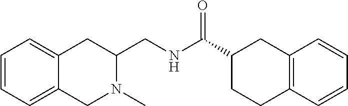 | +++ | ++ |
| 132 | N-((2-Methyl-1,2,3,4- tetrahydroisoquinolin-3- yl)methyl)-2,3-dihydro-1H- indene-2-carboxamide | 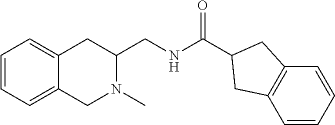 [SMILES: CN1Cc2ccccc2CC1CNC(=O)C1Cc2ccccc2C1] | +++ | ++ |
| 133 | N-((2-Methyl-1,2,3,4- tetrahydroisoquinolin-3- yl)methyl)-1H-indene-2- carboxamide | 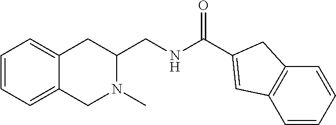 [SMILES: CN1Cc2ccccc2CC1CNC(=O)C1=Cc2ccccc2C1] | +++ | ++ |
| 134 | 4-Ethyl-N-(((3R)-2-methyl- 1,2,3,4- tetrahydroisoquinolin-3- yl)methyl)benzamide | 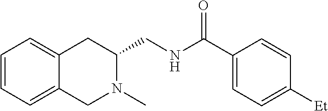 [SMILES: CCc1ccc(C(=O)NC[C@H]2Cc3ccccc3CN2C)cc1] | +++ | ++ |
| 135 | 3-Cyclopentyl-N-(((3R)-2- methyl-1,2,3,4- tetrahydroisoquinolin-3- yl)methyl)propenamide | 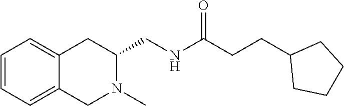 [SMILES: CN1Cc2ccccc2C[C@@H]1CNC(=O)CCC1CCCC1] | +++ | ++ |
| 136 | N-(((3R)-2-Methyl-1,2,3,4- tetrahydroisoquinolin-3- yl)methyl)-1H-indene-2- carboxamide | 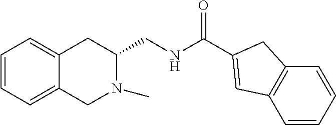 [SMILES: CN1Cc2ccccc2C[C@@H]1CNC(=O)C1=Cc2ccccc2C1] | +++ | ++ |
| 137 | 3-(4-Methylphenyl)-N- (((3R)-2-methyl-1,2,3,4- tetrahydroisoquinolin-3- yl)methyl)propanamide | 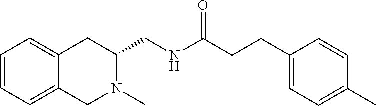 [SMILES: Cc1ccc(CCC(=O)NC[C@H]2Cc3ccccc3CN2C)cc1] | ++ | ++ |
| 138 | 3-(3-Methylphenyl)-N- (((3R)-2-methyl-1,2,3,4- tetrahydroisoquinolin-3- yl)methyl)propenamide | 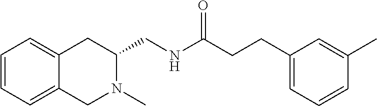 [SMILES: Cc1cccc(CCC(=O)NC[C@H]2Cc3ccccc3CN2C)c1] | +++ | ++ |
| 139 | 3-(3-Chlorophenyl)-N- (((3R)-2-methyl-1,2,3,4- tetrahydroisoquinolin-3- yl)methyl)propanamide | 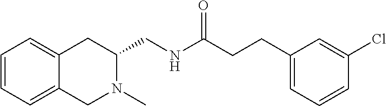 [SMILES: CN1Cc2ccccc2C[C@@H]1CNC(=O)CCc1cccc(Cl)c1] | +++ | ++ |
| 140 | 3-Cyclopentyl-N-(((3S)-2- methyl-1,2,3,4- tetrahydroisoquinolin-3- yl)methyl)propenamide | 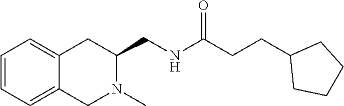 [SMILES: CN1Cc2ccccc2C[C@H]1CNC(=O)CCC1CCCC1] | ++ | ++ |
| 141 | (2R)-N-(((3S)-2-methyl- 1,2,3,4- tetrahydroisoquinolin-3- yl)methyl)-1,2,3,4- tetrahydronaphthalene-2- carboxamide | 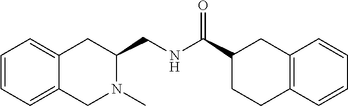 | +++ | ++ |
| 142 | (2S)-N-(((3S)-2-Methyl- 1,2,3,4- tetrahydroisoquinolin-3- yl)methyl)-1,2,3,4- tetrahydronaphthalene-2- carboxamide | 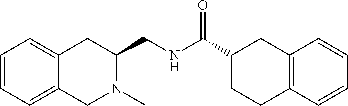 | ++ | ++ |
| 143 | N-(((3S)-2-Methyl-1,2,3,4- tetrahydroisoquinolin-3- yl)methyl)-2,3-dihydro-1H- indene-2-carboxamide | 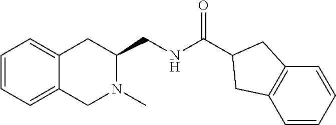 [SMILES: CN1Cc2ccccc2C[C@H]1CNC(=O)C1Cc2ccccc2C1] | +++ | ++ |
| 144 | N-(((3S)-2-methyl-1,2,3,4- tetrahydroisoquinolin-3- yl)methyl)-1H-indene-2- carboxamide | 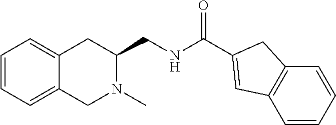 [SMILES: CN1Cc2ccccc2C[C@H]1CNC(=O)C1=Cc2ccccc2C1] | ++ | ++ |
| 145 | 3-(4-Methylphenyl)-N- (((3S)-2-methyl-1,2,3,4- tetrahydroisoquinolin-3- yl)methyl)propanamide | 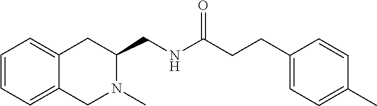 [SMILES: Cc1ccc(CCC(=O)NC[C@@H]2Cc3ccccc3CN2C)cc1] | + | ++ |
| 146 | 3-(3-Methylphenyl)-N- (((3S)-2-methyl-1,2,3,4- tetrahydroisoquinolin-3- yl)methyl)propenamide | 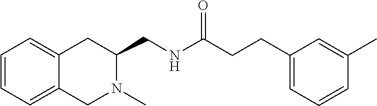 [SMILES: Cc1cccc(CCC(=O)NC[C@@H]2Cc3ccccc3CN2C)c1] | + | ++ |
| 147 | 3-(3-Chlorophenyl)-N- (((3S)-2-methyl-1,2,3,4- tetrahydroisoquinolin-3- yl)methyl)propanamide | 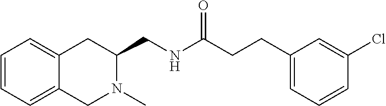 [SMILES: CN1Cc2ccccc2C[C@H]1CNC(=O)CCc1cccc(Cl)c1] | ++ | ++ |
| 148 | N-((2,5-Dimethyl-1,2,3,4- tetrahydroisoquinolin-3- yl)methyl)-3-(3- fluorophenyl)propenamide | 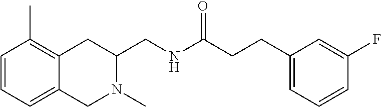 [SMILES: Cc1cccc2c1CC(CNC(=O)CCc1cccc(F)c1)N(C)C2] | ++ | ++ |
| 149 | N-((2,5-Dimethyl-1,2,3,4- tetrahydroisoquinolin-3- yl)methyl)-3- phenylpropanamide | 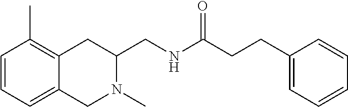 [SMILES: Cc1cccc2c1CC(CNC(=O)CCc1ccccc1)N(C)C2] | ++ | ++ |
| 150 | 3-Cyclohexyl-N-((2,5- dimethyl-1,2,3,4- tetrahydroisoquinolin-3- yl)methyl)propenamide | 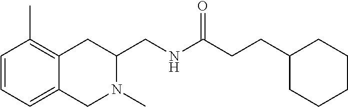 [SMILES: Cc1cccc2c1CC(CNC(=O)CCC1CCCCC1)N(C)C2] | ++ | ++ |
| 151 | N-((2,5-Dimethyl-1,2,3,4- tetrahydroisoquinolin-3- yl)methyl)-2,3-dihydro-1H- indene-2-carboxamide | 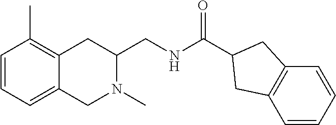 [SMILES: Cc1cccc2c1CC(CNC(=O)C1Cc3ccccc3C1)N(C)C2] | +++ | ++ |
| 152 | N-(((3S)-2,5-dimethyl- 1,2,3,4- tetrahydroisoquinolin-3- yl)methyl)-2,3-dihydro-1H- indene-2-carboxamide | 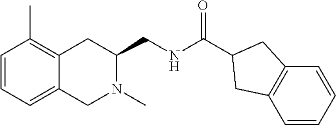 [SMILES: Cc1cccc2c1C[C@@H](CNC(=O)C1Cc3ccccc3C1)N(C)C2] | ++ | ++ |
| 153 | N-((2,6-Dimethyl-1,2,3,4- tetrahydroisoquinolin-3- yl)methyl)-3-(3- fluorophenyl)propanamide | 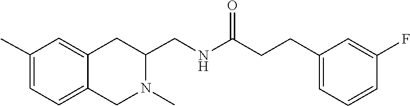 [SMILES: Cc1ccc2c(c1)CC(CNC(=O)CCc1cccc(F)c1)N(C)C2] | ++ | ++ |
| 154 | N-((2,6-Dimethyl-1,2,3,4- tetrahydroisoquinolin-3- yl)methyl)-2,3-dihydro-1H- indene-2-carboxamide | 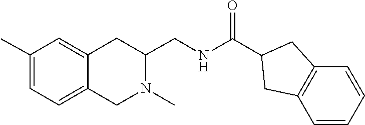 [SMILES: Cc1ccc2c(c1)CC(CNC(=O)C1Cc3ccccc3C1)N(C)C2] | +++ | ++ |
| 155 | N-((2,6-Dimethyl-1,2,3,4- tetrahydroisoquinolin-3- yl)methyl)-1,2,3,4- tetrahydronaphthalene-2- carboxamide | 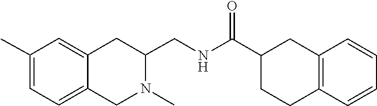 [SMILES: Cc1ccc2c(c1)CC(CNC(=O)C1CCc3ccccc3C1)N(C)C2] | +++ | ++ |
| 156 | N-((2,6-Dimethyl-1,2,3,4- tetrahydroisoquinolin-3- yl)methyl)-5-fluoro-2,3- dihydro-1H-indene-2- carboxamide | 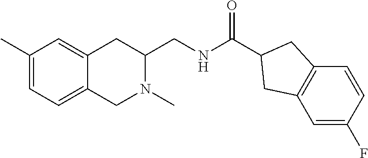 [SMILES: Cc1ccc2c(c1)CC(CNC(=O)C1Cc3ccc(F)cc3C1)N(C)C2] | ++ | ++ |
| 157 | (3S)-N-((2,6-Dimethyl- 1,2,3,4- tetrahydroisoquinolin-3- yl)methyl)-2-methyl- 1,2,3,4- tetrahydroisoquinoline-3- carboxamide | 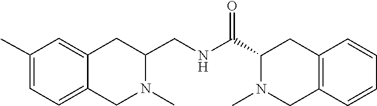 [SMILES: Cc1ccc2c(c1)CC(CNC(=O)[C@@H]1Cc3ccccc3CN1C)N(C)C2] | ++ | ++ |
| 158 | N-((2,6-Dimethyl-1,2,3,4- tetrahydroisoquinolin-3- yl)methyl)-1H-indene-2- carboxamide | 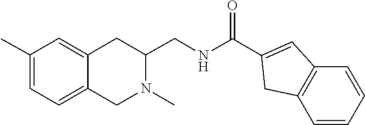 [SMILES: Cc1ccc2c(c1)CC(CNC(=O)C1=Cc3ccccc3C1)N(C)C2] | ++ | ++ |
| 159 | 3,4-Dichloro-N-(((3R)-2,6- dimethyl-1,2,3,4- tetrahydroisoquinolin-3- yl)methyl)benzamide | 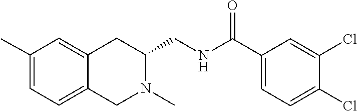 [SMILES: Cc1ccc2c(c1)C[C@H](CNC(=O)c1ccc(Cl)c(Cl)c1)N(C)C2] | ++ | ++ |
| 160 | (3S)-N-(((3R)-2,6- Dimethyl-1,2,3,4- tetrahydroisoquinolin-3- yl)methyl)-2-methyl- 1,2,3,4- tetrahydroisoquinoline-3- carboxamide | 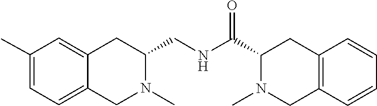 [SMILES: Cc1ccc2c(c1)C[C@H](CNC(=O)[C@@H]1Cc3ccccc3CN1C)N(C)C2] | +++ | ++ |
| 161 | N-(((3R)-2,6-Dimethyl- 1,2,3,4- tetrahydroisoquinolin-3- yl)methyl)naphthalene-2- carboxamide | 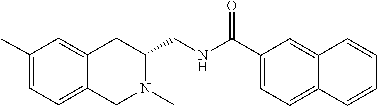 [SMILES: Cc1ccc2c(c1)C[C@H](CNC(=O)c1ccc3ccccc3c1)N(C)C2] | ++ | ++ |
| 162 | (2R)-N-(((3R)-2,6- Dimethyl-1,2,3,4- tetrahydroisoquinolin-3- yl)methyl)-1,2,3,4- tetrahydronaphthalene-2- carboxamide | 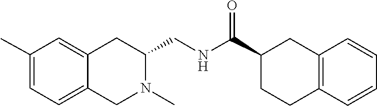 [SMILES: Cc1ccc2c(c1)C[C@H](CNC(=O)[C@@H]1CCc3ccccc3C1)N(C)C2] | ++ | ++ |
| 163 | (2S)-N-(((3R)-2,6- Dimethyl-1,2,3,4- tetrahydroisoquinolin-3- yl)methyl)-1,2,3,4- tetrahydronaphthalene-2- carboxamide | 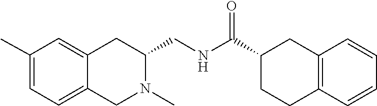 [SMILES: Cc1ccc2c(c1)C[C@H](CNC(=O)[C@H]1CCc3ccccc3C1)N(C)C2] | +++ | ++ |
| 164 | N-(((3S)-2,6-Dimethyl- 1,2,3,4- tetrahydroisoquinolin-3- yl)methyl)-4- (ethylsulfanyl)benzamide | 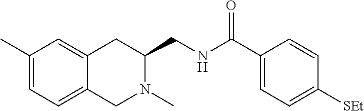 | ++ | ++ |
| 165 | 3,4-Dichloro-N-(((3S)-2,6- dimethyl-1,2,3,4- tetrahydroisoquinolin-3- yl)methyl)benzamide | 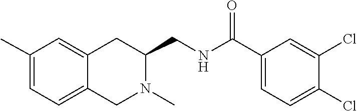 [SMILES: Cc1ccc2c(c1)C[C@@H](CNC(=O)c1ccc(Cl)c(Cl)c1)N(C)C2] | ++ | ++ |
| 166 | N-(((3S)-2,6-Dimethyl- 1,2,3,4- tetrahydroisoquinolin-3- yl)methyl)-3-(3- fluorophenyl)propanamide | 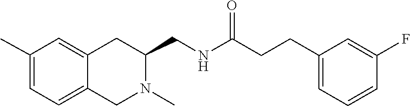 [SMILES: Cc1ccc2c(c1)C[C@@H](CNC(=O)CCc1cccc(F)c1)N(C)C2] | ++ | ++ |
| 167 | N-(((3S)-2,6-Dimethyl- 1,2,3,4- tetrahydroisoquinolin-3- yl)methyl)-3- phenylpropanamide | 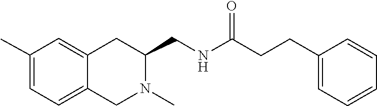 [SMILES: Cc1ccc2c(c1)C[C@@H](CNC(=O)CCc1ccccc1)N(C)C2] | ++ | + |
| 168 | 3-Cyclohexyl-N-(((3S)-2,6- dimethyl-1,2,3,4- tetrahydroisoquinolin-3- yl)methyl)propanamide | 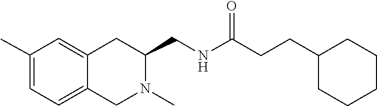 [SMILES: Cc1ccc2c(c1)C[C@@H](CNC(=O)CCC1CCCCC1)N(C)C2] | + | ++ |
| 169 | N-(((3S)-2,6-Dimethyl- 1,2,3,4- tetrahydroisoquinolin-3- yl)methyl)-2,3-dihydro-1H- indene-2-carboxamide | 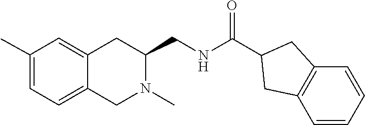 [SMILES: Cc1ccc2c(c1)C[C@@H](CNC(=O)C1Cc3ccccc3C1)N(C)C2] | +++ | ++ |
| 170 | N-(((3S)-2,6-Dimethyl- 1,2,3,4- tetrahydroisoquinolin-3- yl)methyl)-1,2,3,4- tetrahydronaphthalene-2- carboxamide | 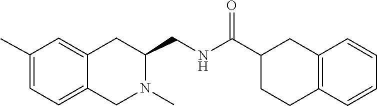 [SMILES: Cc1ccc2c(c1)C[C@@H](CNC(=O)C1CCc3ccccc3C1)N(C)C2] | ++ | ++ |
| 171 | N-(((3S)-2,6-Dimethyl- 1,2,3,4- tetrahydroisoquinolin-3- yl)methyl)-5-fluoro-2,3- dihydro-1H-indene-2- carboxamide | 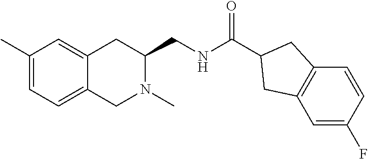 [SMILES: Cc1ccc2c(c1)C[C@@H](CNC(=O)C1Cc3ccc(F)cc3C1)N(C)C2] | ++ | ++ |
| 172 | (3S)-N-(((3S)-2,6- Dimethyl-1,2,3,4- tetrahydroisoquinolin-3- yl)methyl)-2-methyl- 1,2,3,4- tetrahydroisoquinoline-3- carboxamide | 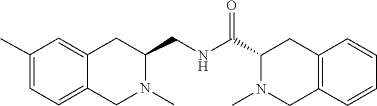 [SMILES: Cc1ccc2c(c1)C[C@@H](CNC(=O)[C@@H]1Cc3ccccc3CN1C)N(C)C2] | ++ | ++ |
| 173 | N-(((3S)-2,6-Dimethyl- 1,2,3,4- tetrahydroisoquinolin-3- yl)methyl)-1H-indene-2- carboxamide | 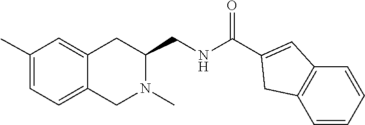 [SMILES: Cc1ccc2c(c1)C[C@@H](CNC(=O)C1=Cc3ccccc3C1)N(C)C2] | ++ | ++ |
| 174 | (2R)-N-(((3S)-2,6- Dimethyl-1,2,3,4- tetrahydroisoquinolin-3- yl)methyl)-1,2,3,4- tetrahydronaphthalene-2- carboxamide | 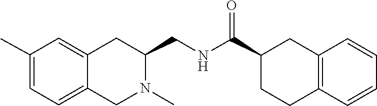 [SMILES: Cc1ccc2c(c1)C[C@@H](CNC(=O)[C@@H]1CCc3ccccc3C1)N(C)C2] | ++ | ++ |
| 175 | (2S)-N-(((3S)-2,6- Dimethyl-1,2,3,4- tetrahydroisoquinolin-3- yl)methyl)-1,2,3,4- tetrahydronaphthalene-2- carboxamide | 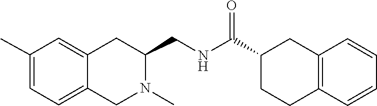 [SMILES: Cc1ccc2c(c1)C[C@@H](CNC(=O)[C@H]1CCc3ccccc3C1)N(C)C2] | ++ | ++ |
| 176 | 3-(3-Fluorophenyl)-N-((6- methoxy-2-methyl-1,2,3,4- tetrahydroisoquinolin-3- yl)methyl)propenamide | 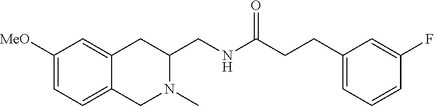 [SMILES: COc1ccc2c(c1)CC(CNC(=O)CCc1cccc(F)c1)N(C)C2] | ++ | ++ |
| 177 | N-((6-Methoxy-2-methyl- 1,2,3,4- tetrahydroisoquinolin-3- yl)methyl)-3- phenylpropanamide | 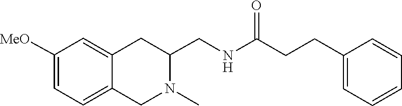 [SMILES: COc1ccc2c(c1)CC(CNC(=O)CCc1ccccc1)N(C)C2] | ++ | ++ |
| 178 | 3-Cyclohexyl-N-((6- methoxy-2-methyl-1,2,3,4- tetrahydroisoquinolin-3- yl)methyl)propenamide | 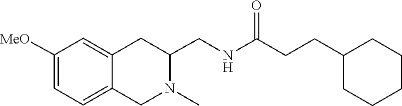 [SMILES: COc1ccc2c(c1)CC(CNC(=O)CCC1CCCCC1)N(C)C2] | ++ | ++ |
| 179 | 3-(3-Fluorophenyl)-N- (((3S)-6-methoxy-2-methyl- 1,2,3,4- tetrahydroisoquinolin-3- yl)methyl)propenamide | 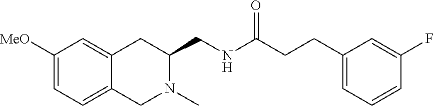 [SMILES: COc1ccc2c(c1)C[C@@H](CNC(=O)CCc1cccc(F)c1)N(C)C2] | + | + |
| 180 | N-(((3S)-6-Methoxy-2- methyl-1,2,3,4- tetrahydroisoquinolin-3- yl)methyl)-3- phenylpropanamide | 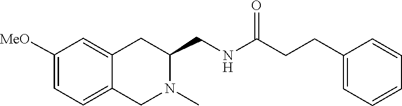 [SMILES: COc1ccc2c(c1)C[C@@H](CNC(=O)CCc1ccccc1)N(C)C2] | + | + |
| 181 | 3-Cyclohexyl-N-(((3S)-6- methoxy-2-methyl-1,2,3,4- tetrahydroisoquinolin-3- yl)methyl)propenamide | 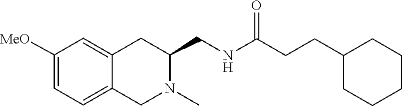 [SMILES: COc1ccc2c(c1)C[C@@H](CNC(=O)CCC1CCCCC1)N(C)C2] | + | ++ |
| 182 | N-(((3R)-2,7-Dimethyl- 1,2,3,4- tetrahydroisoquinolin-3- yl)methyl)-4- (trifluoromethyl)benzamide | 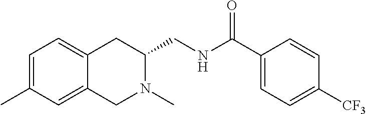 [SMILES: Cc1ccc2c(c1)CN(C)[C@@H](CNC(=O)c1ccc(C(F)(F)F)cc1)C2] | ++ | ++ |
| 183 | N-(((3R)-2,7-Dimethyl- 1,2,3,4- tetrahydroisoquinolin-3- yl)methyl)-4- (ethylsulfanyl)benzamide | 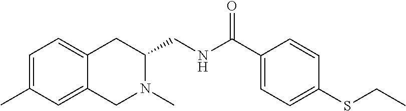 [SMILES: CCSc1ccc(C(=O)NC[C@H]2Cc3ccc(C)cc3CN2C)cc1] | ++ | ++ |
| 184 | 3,4-Dichloro-N-(((3R)-2,7- dimethyl-1,2,3,4- tetrahydroisoquinolin-3- yl)methyl)benzamide | 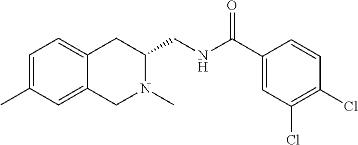 [SMILES: Cc1ccc2c(c1)CN(C)[C@@H](CNC(=O)c1ccc(Cl)c(Cl)c1)C2] | ++ | ++ |
| 185 | N-(((3S)-2,7-Dimethyl- 1,2,3,4- tetrahydroisoquinolin-3- yl)methyl)-4- (trifluoromethyl)benzamide | 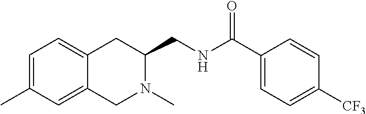 [SMILES: Cc1ccc2c(c1)CN(C)[C@H](CNC(=O)c1ccc(C(F)(F)F)cc1)C2] | + | ++ |
| 186 | N-(((3S)-2,7-Dimethyl- 1,2,3,4- tetrahydroisoquinolin-3- yl)methyl)-4- (ethylsulfanyl)benzamide | 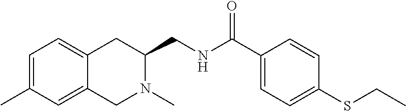 [SMILES: CCSc1ccc(C(=O)NC[C@@H]2Cc3ccc(C)cc3CN2C)cc1] | + | ++ |
| 187 | 3,4-Dichloro-N-(((3S)-2,7- dimethyl-1,2,3,4- tetrahydroisoquinolin-3- yl)methyl)benzamide | 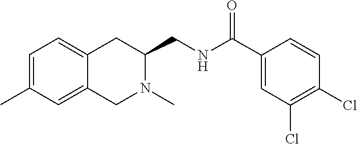 [SMILES: Cc1ccc2c(c1)CN(C)[C@H](CNC(=O)c1ccc(Cl)c(Cl)c1)C2] | + | ++ |
| 188 | N-((5-Chloro-2-methyl- 1,2,3,4- tetrahydroisoquinolin-3- yl)methyl)-1H-indene-2- carboxamide | 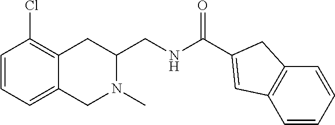 [SMILES: CN1Cc2cccc(Cl)c2CC1CNC(=O)C1=Cc2ccccc2C1] | ++ | ++ |
| 189 | N-(((3R)-5-Chloro-2- methyl-1,2,3,4- tetrahydroisoquinolin-3- yl)methyl)-1H-indene-2- carboxamide | 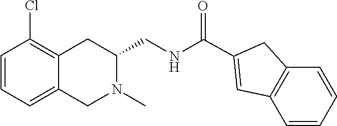 [SMILES: CN1Cc2cccc(Cl)c2C[C@@H]1CNC(=O)C1=Cc2ccccc2C1] | ++ | ++ |
| 190 | (2R)-N-(((3R)-5-chloro-2- methyl-1,2,3,4- tetrahydroisoquinolin-3- yl)methyl)-1,2,3,4- tetrahydronaphthalene-2- carboxamide | 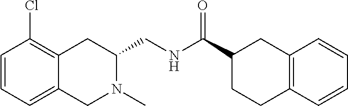 | ++ | ++ |
| 191 | (2S)-N-(((3R)-5-Chloro-2- methyl-1,2,3,4- tetrahydroisoquinolin-3- yl)methyl)-1,2,3,4- tetrahydronaphthalene-2- carboxamide | 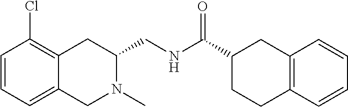 | +++ | ++ |
| 192 | N-(((3S)-5-Chloro-2- methyl-1,2,3,4- tetrahydroisoquinolin-3- yl)methyl)-1H-indene-2- carboxamide | 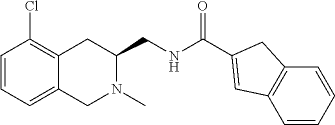 [SMILES: CN1Cc2cccc(Cl)c2C[C@H]1CNC(=O)C1=Cc2ccccc2C1] | + | ++ |
| 193 | (2R)-N-(((3S)-5-Chloro-2- methyl-1,2,3,4- tetrahydroisoquinolin-3- yl)methyl)-1,2,3,4- tetrahydronaphthalene-2- carboxamide | 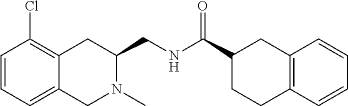 | + | ++ |
| 194 | (2S)-N-(((3S)-5-Chloro-2- methyl-1,2,3,4- tetrahydroisoquinolin-3- yl)methyl)-1,2,3,4- tetrahydronaphthalene-2- carboxamide | 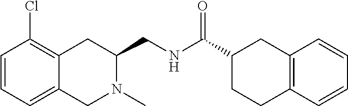 | ++ | ++ |
| 195 | N-(((3S)-5-Chloro-2- methyl-1,2,3,4- tetrahydroisoquinolin-3- yl)methyl)-1,2,3,4- tetrahydronaphthalene-2- carboxamide | 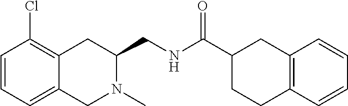 [SMILES: CN1Cc2cccc(Cl)c2C[C@H]1CNC(=O)C1CCc2ccccc2C1] | ++ | ++ |
| 196 | N-(((3S)-5-Chloro-2- methyl-1,2,3,4- tetrahydroisoquinolin-3- yl)methyl)-2,3-dihydro-1H- indene-2-carboxamide | 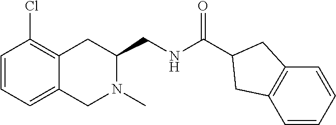 [SMILES: CN1Cc2cccc(Cl)c2C[C@H]1CNC(=O)C1Cc2ccccc2C1] | ++ | ++ |
| 197 | N-((6-Fluoro-2-methyl- 1,2,3,4- tetrahydroisoquinolin-3- yl)methyl)-2,3-dihydro-1H- indene-2-carboxamide | 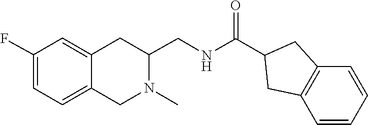 [SMILES: CN1Cc2ccc(F)cc2CC1CNC(=O)C1Cc2ccccc2C1] | ++ | ++ |
| 198 | N-((6-Fluoro-2-methyl- 1,2,3,4- tetrahydroisoquinolin-3- yl)methyl)-1,2,3,4- tetrahydronaphthalene-2- carboxamide | 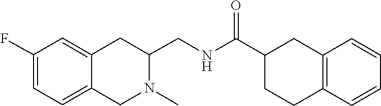 [SMILES: CN1Cc2ccc(F)cc2CC1CNC(=O)C1CCc2ccccc2C1] | ++ | ++ |
| 199 | trans-N-((6-Fluoro-2- methyl-1,2,3,4- tetrahydroisoquinolin-3- yl)methyl)-2- phenylcyclopropane-1- carboxamide | 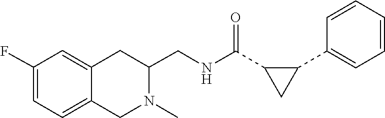 [SMILES: CN1Cc2ccc(F)cc2CC1CNC(=O)[C@H]1C[C@H]1c1ccccc1] | ++ | ++ |
| 200 | N-((6-Fluoro-2-methyl- 1,2,3,4- tetrahydroisoquinolin-3- yl)methyl)-1H-indene-2- carboxamide | 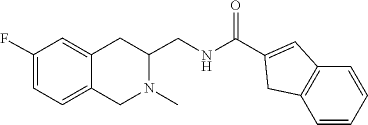 [SMILES: CN1Cc2ccc(F)cc2CC1CNC(=O)C1=Cc2ccccc2C1] | ++ | ++ |
| 201 | 4-Ethyl-N-(((3R)-6-fluoro- 2-methyl-1,2,3,4- tetrahydroisoquinolin-3- yl)methyl)benzamide | 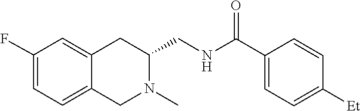 [SMILES: CCc1ccc(C(=O)NC[C@H]2Cc3cc(F)ccc3CN2C)cc1] | ++ | ++ |
| 202 | N-(((3R)-6-Fluoro-2- methyl-1,2,3,4- tetrahydroisoquinolin-3- yl)methyl)-4- (trifluoromethyl)benzamide | 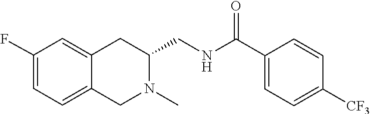 [SMILES: CN1Cc2ccc(F)cc2C[C@@H]1CNC(=O)c1ccc(C(F)(F)F)cc1] | ++ | ++ |
| 203 | N-(((3R)-6-Fluoro-2- methyl-1,2,3,4- tetrahydroisoquinolin-3- yl)methyl)-3-(3- fluorophenyl)propanamide | 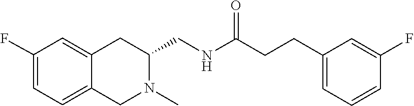 [SMILES: CN1Cc2ccc(F)cc2C[C@@H]1CNC(=O)CCc1cccc(F)c1] | ++ | ++ |
| 204 | N-(((3R)-6-Fluoro-2- methyl-1,2,3,4- tetrahydroisoquinolin-3- yl)methyl)-1H-indene-2- carboxamide | 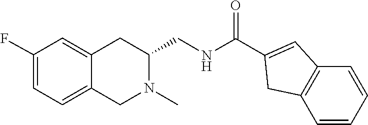 [SMILES: CN1Cc2ccc(F)cc2C[C@@H]1CNC(=O)C1=Cc2ccccc2C1] | ++ | ++ |
| 205 | 4-Ethyl-N-(((3S)-6-fluoro- 2-methyl-1,2,3,4- tetrahydroisoquinolin-3- yl)methyl)benzamide | 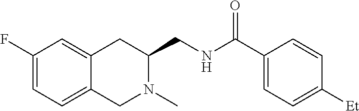 [SMILES: CCc1ccc(C(=O)NC[C@@H]2Cc3cc(F)ccc3CN2C)cc1] | + | ++ |
| 206 | N-(((3S)-6-Fluoro-2- methyl-1,2,3,4- tetrahydroisoquinolin-3- yl)methyl)-4- (trifluoromethyl)benzamide | 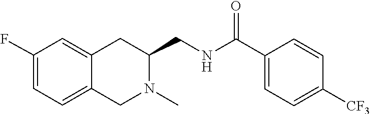 [SMILES: CN1Cc2ccc(F)cc2C[C@H]1CNC(=O)c1ccc(C(F)(F)F)cc1] | ++ | ++ |
| 207 | N-(((3S)-6-Fluoro-2- methyl-1,2,3,4- tetrahydroisoquinolin-3- yl)methyl)-3-(3- fluorophenyl)propanamide | 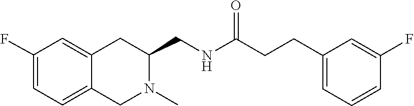 [SMILES: CN1Cc2ccc(F)cc2C[C@H]1CNC(=O)CCc1cccc(F)c1] | + | ++ |
| 208 | N-(((3S)-6-Fluoro-2- methyl-1,2,3,4- tetrahydroisoquinolin-3- yl)methyl)-2,3-dihydro-1H- indene-2-carboxamide | 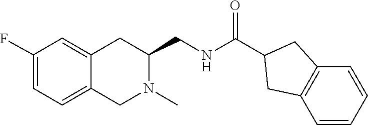 [SMILES: CN1Cc2ccc(F)cc2C[C@H]1CNC(=O)C1Cc2ccccc2C1] | ++ | ++ |
| 209 | N-(((3S)-6-Fluoro-2- methyl-1,2,3,4- tetrahydroisoquinolin-3- yl)methyl)-1,2,3,4- tetrahydronaphthalene-2- carboxamide | 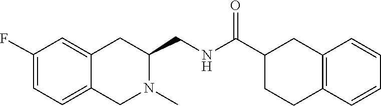 [SMILES: CN1Cc2ccc(F)cc2C[C@H]1CNC(=O)C1CCc2ccccc2C1] | +++ | ++ |
| 210 | trans-N-(((3S)-6-Fluoro-2- methyl-1,2,3,4- tetrahydroisoquinolin-3- yl)methyl)-2- phenylcyclopropane-1- carboxamide | 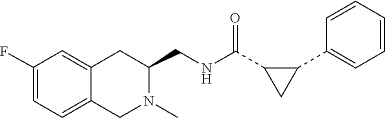 [SMILES: CN1Cc2ccc(F)cc2C[C@H]1CNC(=O)[C@H]1C[C@H]1c1ccccc1] | ++ | ++ |
| 211 | N-(((3S)-6-Fluoro-2- methyl-1,2,3,4- tetrahydroisoquinolin-3- yl)methyl)-1H-indene-2- carboxamide | 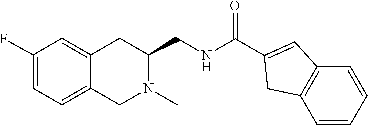 [SMILES: CN1Cc2ccc(F)cc2C[C@H]1CNC(=O)C1=Cc2ccccc2C1] | ++ | ++ |
| 212 | N-((6-Chloro-2-methyl- 1,2,3,4- tetrahydroisoquinolin-3- yl)methyl)-3- cyclohexylpropanamide | 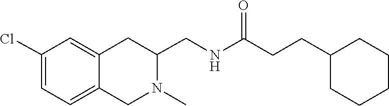 [SMILES: CN1Cc2ccc(Cl)cc2CC1CNC(=O)CCC1CCCCC1] | ++ | ++ |
| 213 | N-((6-Chloro-2-methyl- 1,2,3,4- tetrahydroisoquinolin-3- yl)methyl)-3- phenylpropanamide | 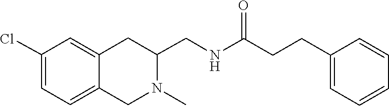 [SMILES: CN1Cc2ccc(Cl)cc2CC1CNC(=O)CCc1ccccc1] | ++ | ++ |
| 214 | N-((6-Chloro-2-methyl- 1,2,3,4- tetrahydroisoquinolin-3- yl)methyl)-1,2,3,4- tetrahydronaphthalene-2- carboxamide | 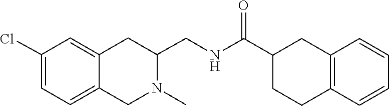 [SMILES: CN1Cc2ccc(Cl)cc2CC1CNC(=O)C1CCc2ccccc2C1] | +++ | ++ |
| 215 | N-((6-Chloro-2-methyl- 1,2,3,4- tetrahydroisoquinolin-3- yl)methyl)-2,3-dihydro-1H- indene-2-carboxamide | 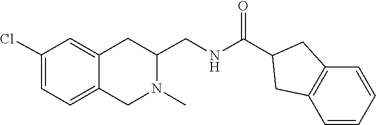 [SMILES: CN1Cc2ccc(Cl)cc2CC1CNC(=O)C1Cc2ccccc2C1] | +++ | ++ |
| 216 | N-((6-Chloro-2-methyl- 1,2,3,4- tetrahydroisoquinolin-3- yl)methyl)-1H-indene-2- carboxamide | 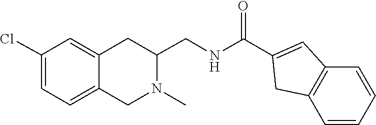 [SMILES: CN1Cc2ccc(Cl)cc2CC1CNC(=O)C1=Cc2ccccc2C1] | ++ | ++ |
| 217 | N-(((3R)-6-Chloro-2- methyl-1,2,3,4- tetrahydroisoquinolin-3- yl)methyl)-4- ethylbenzamide | 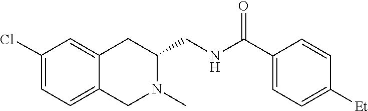 [SMILES: CCc1ccc(C(=O)NC[C@H]2Cc3cc(Cl)ccc3CN2C)cc1] | ++ | ++ |
| 218 | N-(((3R)-6-Chloro-2- methyl-1,2,3,4- tetrahydroisoquinolin-3- yl)methyl)-4- (ethylsulfanyl)benzamide | 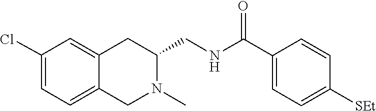 | ++ | ++ |
| 219 | 3,4-Dichloro-N-(((3R)-6- chloro-2-methyl-1,2,3,4- tetrahydroisoquinolin-3- yl)methyl)benzamide | 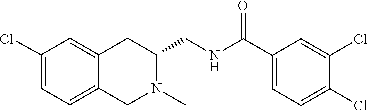 [SMILES: CN1Cc2ccc(Cl)cc2C[C@@H]1CNC(=O)c1ccc(Cl)c(Cl)c1] | ++ | ++ |
| 220 | N-(((3R)-6-Chloro-2- methyl-1,2,3,4- tetrahydroisoquinolin-3- yl)methyl)-1- benzothiophene-2- carboxamide | 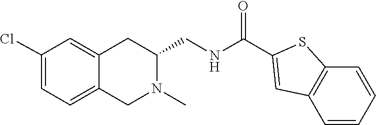 [SMILES: CN1Cc2ccc(Cl)cc2C[C@@H]1CNC(=O)c1cc2ccccc2s1] | ++ | ++ |
| 221 | N-(((3R)-6-Chloro-2- methyl-1,2,3,4- tetrahydroisoquinolin-3- yl)methyl)-1- benzothiophene-3- carboxamide | 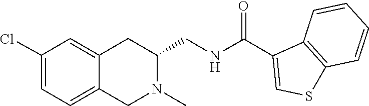 [SMILES: CN1Cc2ccc(Cl)cc2C[C@@H]1CNC(=O)c1csc2ccccc12] | ++ | ++ |
| 222 | N-(((3R)-6-Chloro-2- methyl-1,2,3,4- tetrahydroisoquinolin-3- yl)methyl)-1- benzothiophene-4- carboxamide | 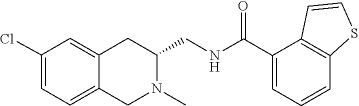 [SMILES: CN1Cc2ccc(Cl)cc2C[C@@H]1CNC(=O)c1cccc2sccc12] | + | ++ |
| 223 | N-(((3R)-6-Chloro-2- methyl-1,2,3,4- tetrahydroisoquinolin-3- yl)methyl)-1- benzothiophene-5- carboxamide | 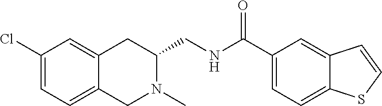 [SMILES: CN1Cc2ccc(Cl)cc2C[C@@H]1CNC(=O)c1ccc2sccc2c1] | ++ | ++ |
| 224 | N-(((3R)-6-Chloro-2- methyl-1,2,3,4- tetrahydroisoquinolin-3- yl)methyl)-6- (trifluoromethyl)-1- benzothiophene-2- carboxamide | 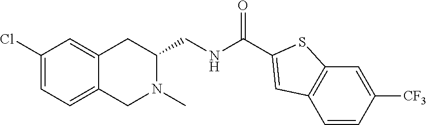 [SMILES: CN1Cc2ccc(Cl)cc2C[C@@H]1CNC(=O)c1cc2ccc(C(F)(F)F)cc2s1] | + | ++ |
| 225 | N-(((3R)-6-Chloro-2- methyl-1,2,3,4- tetrahydroisoquinolin-3- yl)methyl)-1H-indene-2- carboxamide | 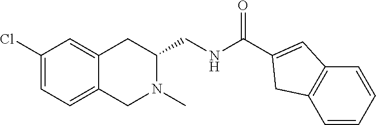 [SMILES: CN1Cc2ccc(Cl)cc2C[C@@H]1CNC(=O)C1=Cc2ccccc2C1] | ++ | ++ |
| 226 | (2R)-N-(((3R)-6-Chloro-2- methyl-1,2,3,4- tetrahydroisoquinolin-3- yl)methyl)-1,2,3,4- tetrahydronaphthalene-2- carboxamide | 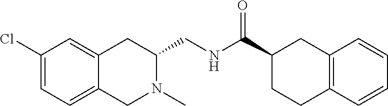 [SMILES: CN1Cc2ccc(Cl)cc2C[C@@H]1CNC(=O)[C@@H]1CCc2ccccc2C1] | ++ | ++ |
| 227 | (2S)-N-(((3R)-6-Chloro-2- methyl-1,2,3,4- tetrahydroisoquinolin-3- yl)methyl)-1,2,3,4- tetrahydronaphthalene-2- carboxamide | 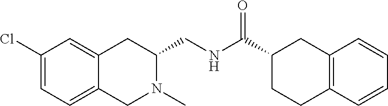 [SMILES: CN1Cc2ccc(Cl)cc2C[C@@H]1CNC(=O)[C@H]1CCc2ccccc2C1] | ++ | ++ |
| 228 | N-(((3S)-6-Chloro-2- methyl-1,2,3,4- tetrahydroisoquinolin-3- yl)methyl)-4- ethylbenzamide | 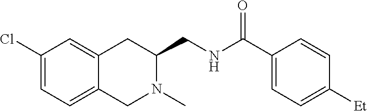 [SMILES: CCc1ccc(C(=O)NC[C@@H]2Cc3cc(Cl)ccc3CN2C)cc1] | ++ | ++ |
| 229 | N-(((3S)-6-Chloro-2- methyl-1,2,3,4- tetrahydroisoquinolin-3- yl)methyl)-4- (ethylsulfanyl)benzamide | 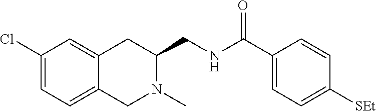 | ++ | ++ |
| 230 | 3,4-Dichloro-N-(((3S)-6- chloro-2-methyl-1,2,3,4- tetrahydroisoquinolin-3- yl)methyl)benzamide | 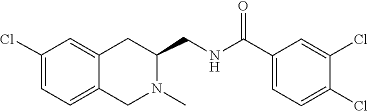 [SMILES: CN1Cc2ccc(Cl)cc2C[C@H]1CNC(=O)c1ccc(Cl)c(Cl)c1] | ++ | ++ |
| 231 | N-(((3S)-6-Chloro-2- methyl-1,2,3,4- tetrahydroisoquinolin-3- yl)methyl)-4- (trifluoromethyl)benzamide | 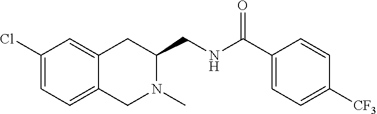 [SMILES: CN1Cc2ccc(Cl)cc2C[C@H]1CNC(=O)c1ccc(C(F)(F)F)cc1] | ++ | ++ |
| 232 | N-(((3S)-6-Chloro-2- methyl-1,2,3,4- tetrahydroisoquinolin-3- yl)methyl)-3-(3- fluorophenyl)propanamide | 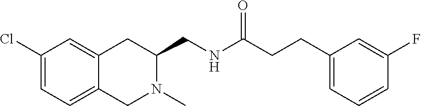 [SMILES: CN1Cc2ccc(Cl)cc2C[C@H]1CNC(=O)CCc1cccc(F)c1] | ++ | ++ |
| 233 | N-(((3S)-6-Chloro-2- methyl-1,2,3,4- tetrahydroisoquinolin-3- yl)methyl)-3- cyclohexylpropanamide | 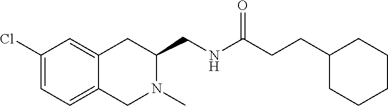 [SMILES: CN1Cc2ccc(Cl)cc2C[C@H]1CNC(=O)CCC1CCCCC1] | ++ | ++ |
| 234 | N-(((3S)-6-Chloro-2- methyl-1,2,3,4- tetrahydroisoquinolin-3- yl)methyl)-3- phenylpropanamide | 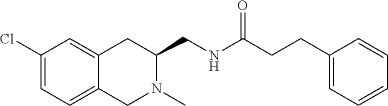 [SMILES: CN1Cc2ccc(Cl)cc2C[C@H]1CNC(=O)CCc1ccccc1] | ++ | ++ |
| 235 | N-(((3S)-6-Chloro-2- methyl-1,2,3,4- tetrahydroisoquinolin-3- yl)methyl)-1,2,3,4- tetrahydronaphthalene-2- carboxamide | 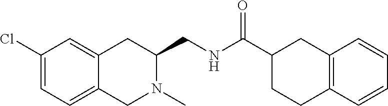 [SMILES: CN1Cc2ccc(Cl)cc2C[C@H]1CNC(=O)C1CCc2ccccc2C1] | ++ | ++ |
| 236 | trans-N-(((3S)-6-Chloro-2- methyl-1,2,3,4- tetrahydroisoquinolin-3- yl)methyl)-2- phenylcyclopropane-1- carboxamide | 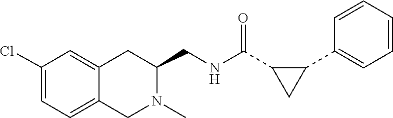 [SMILES: CN1Cc2ccc(Cl)cc2C[C@H]1CNC(=O)[C@H]1C[C@H]1c1ccccc1] | ++ | ++ |
| 237 | N-(((3S)-6-Chloro-2- methyl-1,2,3,4- tetrahydroisoquinolin-3- yl)methyl)-2,3-dihydro-1H- indene-2-carboxamide | 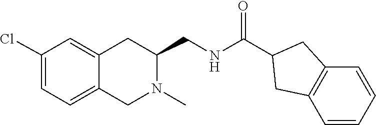 [SMILES: CN1Cc2ccc(Cl)cc2C[C@H]1CNC(=O)C1Cc2ccccc2C1] | ++ | ++ |
| 238 | (2R)-N-(((3S)-6-Chloro-2- methyl-1,2,3,4- tetrahydroisoquinolin-3- yl)methyl)-1,2,3,4- tetrahydronaphthalene-2- carboxamide | 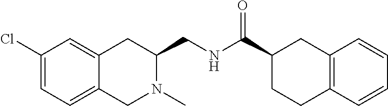 [SMILES: CN1Cc2ccc(Cl)cc2C[C@H]1CNC(=O)[C@@H]1CCc2ccccc2C1] | ++ | ++ |
| 239 | (2S)-N-(((3S)-6-Chloro-2- methyl-1,2,3,4- tetrahydroisoquinolin-3- yl)methyl)-1,2,3,4- tetrahydronaphthalene-2- carboxamide | 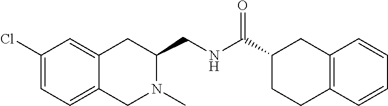 [SMILES: CN1Cc2ccc(Cl)cc2C[C@H]1CNC(=O)[C@H]1CCc2ccccc2C1] | ++ | ++ |
| 240 | N-((6-Bromo-2-methyl- 1,2,3,4- tetrahydroisoquinolin-3- yl)methyl)-2,3-dihydro-1H- indene-2-carboxamide | 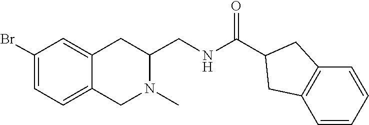 [SMILES: CN1Cc2ccc(Br)cc2CC1CNC(=O)C1Cc2ccccc2C1] | ++ | ++ |
| 241 | N-((6-Bromo-2-methyl- 1,2,3,4- tetrahydroisoquinolin-3- yl)methyl)-1,2,3,4- tetrahydronaphthalene-2- carboxamide | 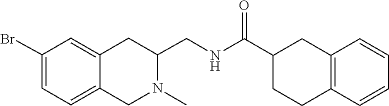 [SMILES: CN1Cc2ccc(Br)cc2CC1CNC(=O)C1CCc2ccccc2C1] | ++ | ++ |
| 242 | N-(((3R)-6-Bromo-2- methyl-1,2,3,4- tetrahydroisoquinolin-3- yl)methyl)-1,2,3,4- tetrahydronaphthalene-2- carboxamide | 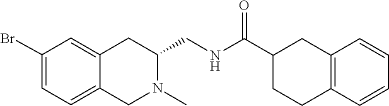 [SMILES: CN1Cc2ccc(Br)cc2C[C@@H]1CNC(=O)C1CCc2ccccc2C1] | +++ | ++ |
| 243 | trans-N-(((3R)-6-Bromo-2- methyl-1,2,3,4- tetrahydroisoquinolin-3- yl)methyl)-2- phenylcyclopropane-1- carboxamide | 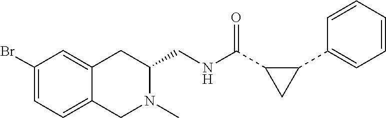 [SMILES: CN1Cc2ccc(Br)cc2C[C@@H]1CNC(=O)[C@H]1C[C@H]1c1ccccc1] | ++ | ++ |
| 244 | N-(((3S)-6-Bromo-2- methyl-1,2,3,4- tetrahydroisoquinolin-3- yl)methyl)-2,3-dihydro-1H- indene-2-carboxamide | 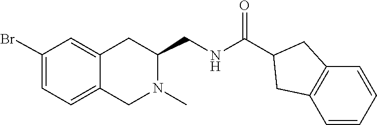 [SMILES: CN1Cc2ccc(Br)cc2C[C@H]1CNC(=O)C1Cc2ccccc2C1] | ++ | ++ |
| 245 | N-(((3S)-6-Bromo-2- methyl-1,2,3,4- tetrahydroisoquinolin-3- yl)methyl)-1,2,3,4- tetrahydronaphthalene-2- carboxamide | 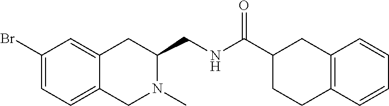 [SMILES: CN1Cc2ccc(Br)cc2C[C@H]1CNC(=O)C1CCc2ccccc2C1] | ++ | ++ |
| 246 | N-(((3R)-7-Fluoro-2- methyl-1,2,3,4- tetrahydroisoquinolin-3- yl)methyl)-4- (trifluoromethyl)benzamide | 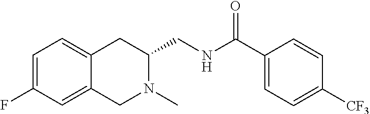 [SMILES: CN1Cc2cc(F)ccc2C[C@@H]1CNC(=O)c1ccc(C(F)(F)F)cc1] | ++ | ++ |
| 247 | 3,4-Dichloro-N-(((3R)-7- fluoro-2-methyl-1,2,3,4- tetrahydroisoquinolin-3- yl)methyl)benzamide | 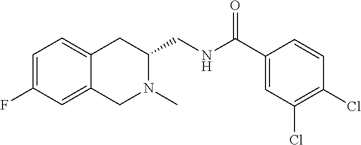 [SMILES: CN1Cc2cc(F)ccc2C[C@@H]1CNC(=O)c1ccc(Cl)c(Cl)c1] | ++ | ++ |
| 248 | N-(((3S)-7-Fluoro-2- methyl-1,2,3,4- tetrahydroisoquinolin-3- yl)methyl)-4- (trifluoromethyl)benzamide | 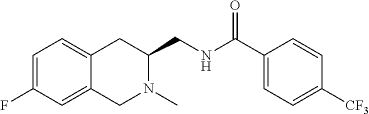 [SMILES: CN1Cc2cc(F)ccc2C[C@H]1CNC(=O)c1ccc(C(F)(F)F)cc1] | ++ | ++ |
| 249 | 3,4-Dichloro-N-(((3S)-7- fluoro-2-methyl-1,2,3,4- tetrahydroisoquinolin-3- yl)methyl)benzamide | 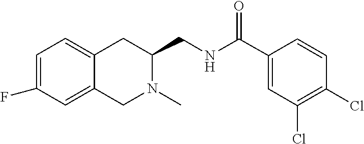 [SMILES: CN1Cc2cc(F)ccc2C[C@H]1CNC(=O)c1ccc(Cl)c(Cl)c1] | ++ | ++ |
| 250 | N-(((3R)-7-Chloro-2- methyl-1,2,3,4- tetrahydroisoquinolin-3- yl)methyl)-4- ethylbenzamide | 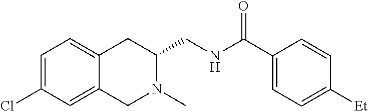 [SMILES: CCc1ccc(C(=O)NC[C@H]2Cc3ccc(Cl)cc3CN2C)cc1] | ++ | ++ |
| 251 | N-(((3S)-7-Chloro-2- methyl-1,2,3,4- tetrahydroisoquinolin-3- yl)methyl)-4- (trifluoromethyl)benzamide | 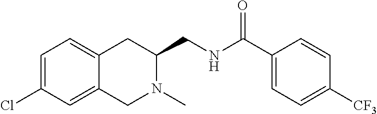 [SMILES: CN1Cc2cc(Cl)ccc2C[C@H]1CNC(=O)c1ccc(C(F)(F)F)cc1] | +++ | ++ |
| 252 | N-(((3S)-7-Chloro-2- methyl-1,2,3,4- tetrahydroisoquinolin-3- yl)methyl)-4- ethylbenzamide | 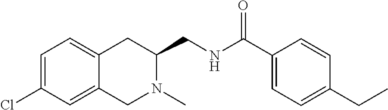 [SMILES: CCc1ccc(C(=O)NC[C@@H]2Cc3ccc(Cl)cc3CN2C)cc1] | +++ | ++ |

Table 2 below shows EC₅₀ values and % of inhibition of cAMP production associated with NOP activation of exemplary compounds. EC₅₀ values are presented in three ranges, i.e., +: more than 100 nM to 2. μM, ++: 1 to 100 nM, and +++: less than 1 nM. Inhibition percentages are presented in two ranges, i.e., ++: >50% and +: 10 to 50%.

TABLE 2

| | NOP cAMP | NOP % inhibition |
|---|---|---|
| No. | EC₅₀ (nM) | of cAMP production |
| 1 | ++ | ++ |
| 4 | ++ | ++ |
| 5 | +++ | ++ |
| 7 | ++ | ++ |
| 12 | ++ | ++ |
| 13 | ++ | ++ |
| 14 | ++ | ++ |
| 22 | +++ | ++ |
| 24 | ++ | ++ |
| 61 | ++ | ++ |
| 62 | ++ | ++ |
| 69 | ++ | ++ |
| 70 | + | ++ |
| 71 | ++ | ++ |
| 72 | +++ | + |
| 73 | +++ | ++ |
| 74 | ++ | ++ |
| 76 | +++ | + |
| 77 | +++ | + |
| 78 | + | ++ |
| 79 | ++ | ++ |
| 80 | ++ | ++ |
| 83 | +++ | + |
| 84 | +++ | + |
| 85 | +++ | + |
| 87 | ++ | ++ |
| 89 | + | ++ |
| 94 | +++ | + |
| 96 | + | ++ |
| 97 | ++ | ++ |
| 108 | + | ++ |
| 111 | ++ | ++ |
| 128 | ++ | ++ |
| 188 | +++ | + |

Table 3 below shows NOP activation by exemplary compounds and % of inhibition of CAMP production at 3 μM and 30 μM. Activation of NOP is indicated by EC₅₀ and % of inhibition of CAMP production. Percentages of inhibition are presented in two ranges, i.e., ++: >50% and +: 10 to 50%.

TABLE 3

| | % of | % of | | % of | % of |
|---|---|---|---|---|---|
| | inhibiton | inhibiton | | inhibiton | inhibiton |
| | of cAMP | of cAMP | | of cAMP | of cAMP |
| | production | production | | production | production |
| No. | at 30 μM | at 3 μM | No. | at 30 μM | at 3 μM |
| 10 μM | ++ | | 43 | ++ | ++ |
| Orphanin | | | | | |
| FQ | | | | | |
| 2 | ++ | ++ | 44 | ++ | ++ |
| 3 | ++ | ++ | 45 | ++ | ++ |
| 6 | ++ | ++ | 46 | ++ | ++ |
| 15 | ++ | ++ | 47 | ++ | ++ |
| 16 | ++ | ++ | 50 | ++ | ++ |
| 17 | ++ | ++ | 51 | ++ | ++ |
| 18 | ++ | ++ | 53 | ++ | ++ |
| 19 | ++ | ++ | 59 | ++ | ++ |
| 20 | ++ | ++ | 63 | ++ | ++ |
| 22 | ++ | ++ | 65 | ++ | + |
| 23 | ++ | ++ | 66 | ++ | + |
| 24 | ++ | ++ | 67 | ++ | ++ |
| 25 | ++ | ++ | 68 | ++ | ++ |
| 26 | ++ | ++ | 69 | + | + |
| 27 | ++ | ++ | 75 | ++ | ++ |
| 28 | + | + | 81 | + | + |
| 29 | ++ | ++ | 82 | + | + |
| 31 | ++ | ++ | 83 | ++ | ++ |
| 34 | ++ | ++ | 84 | + | + |
| 35 | ++ | ++ | 87 | ++ | ++ |
| 36 | ++ | ++ | 88 | ++ | ++ |
| 40 | ++ | ++ | 89 | ++ | ++ |
| 41 | ++ | ++ | 90 | ++ | ++ |
| 42 | ++ | ++ | 91 | ++ | ++ |
| 92 | ++ | ++ | 137 | ++ | ++ |
| 93 | ++ | ++ | 138 | ++ | ++ |
| 94 | ++ | ++ | 139 | ++ | ++ |
| 95 | ++ | ++ | 141 | ++ | ++ |
| 98 | ++ | + | 142 | ++ | ++ |
| 99 | ++ | + | 144 | ++ | ++ |
| 100 | + | + | 148 | ++ | ++ |
| 101 | ++ | ++ | 149 | ++ | ++ |
| 102 | ++ | ++ | 150 | ++ | ++ |
| 103 | ++ | ++ | 151 | ++ | ++ |
| 104 | ++ | ++ | 152 | ++ | ++ |
| 105 | ++ | ++ | 153 | ++ | + |
| 106 | ++ | ++ | 155 | ++ | + |
| 108 | ++ | + | 156 | ++ | + |
| 109 | ++ | ++ | 158 | ++ | ++ |
| 111 | ++ | ++ | 159 | ++ | ++ |
| 113 | ++ | ++ | 162 | ++ | ++ |
| 115 | ++ | ++ | 163 | ++ | ++ |
| 129 | ++ | ++ | 165 | ++ | ++ |
| 130 | ++ | ++ | 171 | + | + |
| 131 | ++ | ++ | 176 | + | + |
| 133 | ++ | ++ | 178 | + | + |
| 134 | ++ | ++ | 190 | ++ | ++ |
| 135 | ++ | ++ | 191 | ++ | ++ |
| 136 | ++ | ++ | 194 | ++ | ++ |
| 196 | ++ | ++ | 214 | ++ | ++ |
| 197 | ++ | ++ | 215 | ++ | ++ |
| 198 | ++ | ++ | 225 | ++ | + |
| 202 | ++ | ++ | 227 | ++ | ++ |
| 203 | ++ | ++ | 235 | ++ | ++ |
| 209 | ++ | ++ | 237 | ++ | ++ |
| 210 | ++ | ++ | 239 | ++ | ++ |
| 212 | ++ | + | 240 | + | + |
| 213 | ++ | ++ | 241 | ++ | + |

Described below are procedures used to synthesize the above-described 252 compounds.

All the reagents and solvents were purchased from commercial sources and used without further purification unless otherwise indication. All the reactions were carried out under dry nitrogen or argon atmosphere and monitored by thin layer chromatography (TLC) using Merck Silica gel 60 F₂₅₄ glass-backed plate. Column chromatography was performed by Merck silica gel 60 (0.040-0.063 mm, 230-400 mesh). ¹H NMR spectra were measured by Varian Mercury-300, Varian Mercury-400, Bruker Avance-400, and Bruker Avance-600 spectrometers, and the chemical shifts (8) were reported in parts per million (ppm) relative to the resonance of the solvent peak. Multiplicities are reported with the following abbreviations: s (singlet), d (doublet), t (triplet), q (quartet), quin (quintet), m (multiplet), or br (broad). Low-resolution mass spectra were measured by HP Hewlett Packard 1100 series.

The following six schemes described the synthesis of the exemplary compounds.

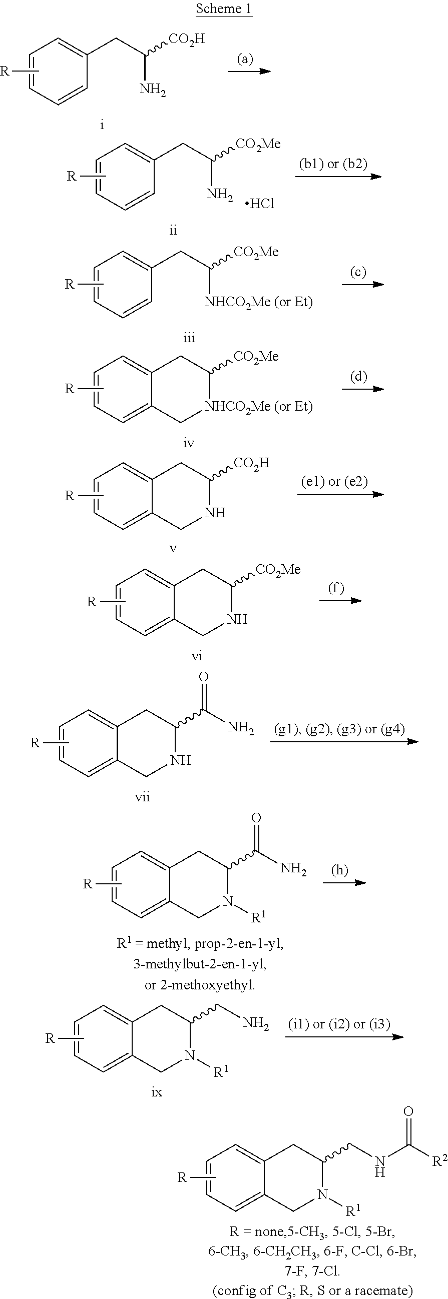

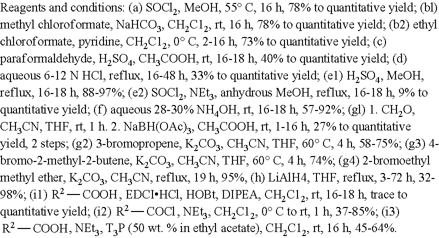

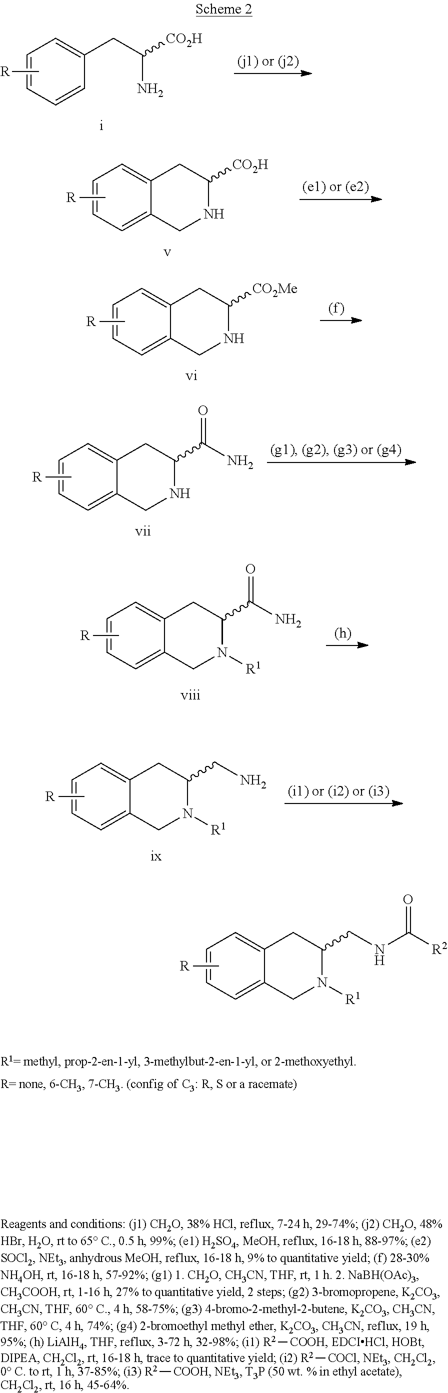

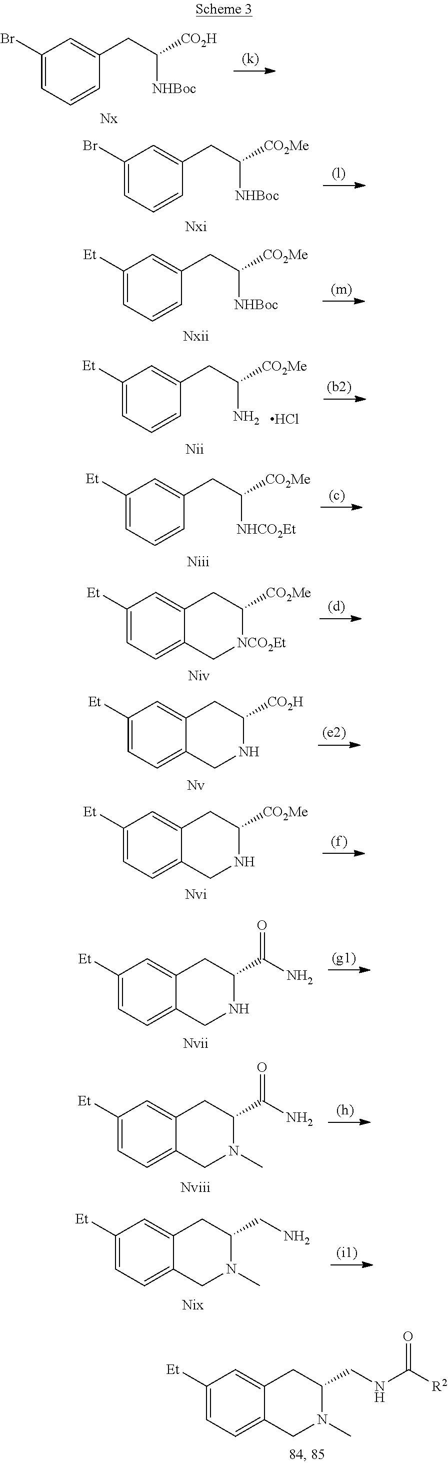

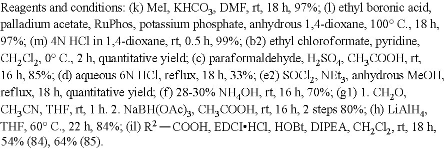

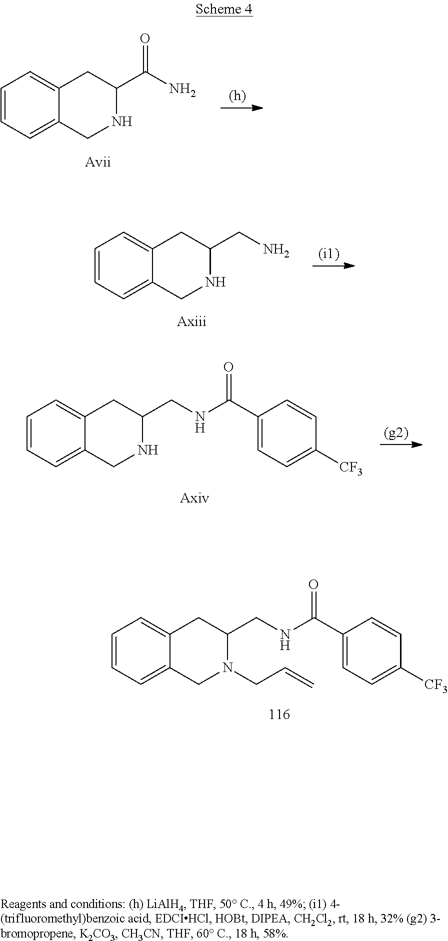 [SMILES: NC(=O)C1Cc2ccccc2CN1 ; NCC1Cc2ccccc2CN1 ; O=C(NCC1Cc2ccccc2CN1)c1ccc(C(F)(F)F)cc1 ; C=CCN1Cc2ccccc2CC1CNC(=O)c1ccc(C(F)(F)F)cc1]

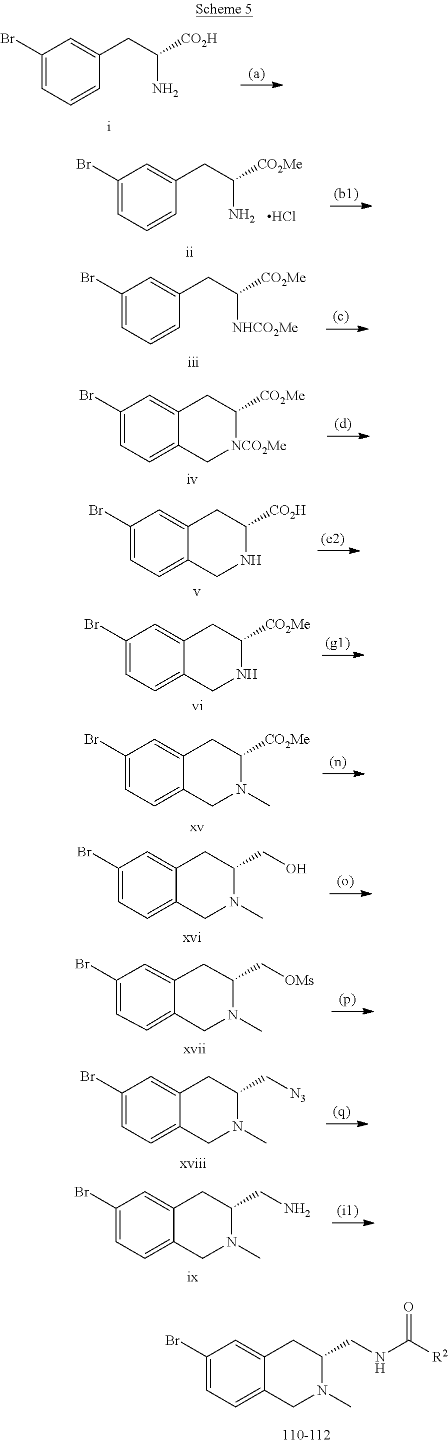

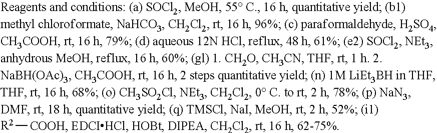

TABLE 4
Intermediates:

| | |
|---|---|
| | ix |
| 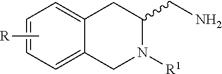 | |

| entry | | | | entry | | | |
|---|---|---|---|---|---|---|---|
| Xstart | | | con- | Xstart | | | con- |
| − | | | fig | − | | | fig |
| Xend | R | R¹ | (C₃) | Xend | R | R¹ | (C₃) |
| Avii − | none | CH₃ | rac | avii − | 5-Cl | CH₃ | S |
| Aix^{a} | | | | aix^{a} | | | |
| Bi − | none | CH₃ | R | bi − | 6-CH₃ | CH₃ | S |
| Bix^{a} | | | | bix^{a} | | | |
| Cv − | none | CH₃ | S | cv − | 6- | CH₃ | rac |
| Cix^{a} | | | | cix^{b} | OCH₃ | | |
| Di − | 5-CH₃ | CH₃ | R | dv − | 6- | CH₃ | S |
| Dix^{a} | | | | dix^{b} | OCH₃ | | |
| Ei − | 5-CH₃ | CH₃ | S | ei − | 6-F | CH₃ | S |
| Eix^{a} | | | | eix^{a} | | | |
| Fi − | 5-Cl | CH₃ | rac | fi − | 6-Cl | CH₃ | S |
| Fix^{a} | | | | fix^{a} | | | |
| Gi − | 5-Cl | CH₃ | R | gi − | 6-Br | CH₃ | rac |
| Gix^{a} | | | | gix^{d} | | | |
| Hi − | 5-Br | CH₃ | rac | hi − | 6-Br | CH₃ | S |
| Hix^{a} | | | | hix^{d} | | | |
| Ii − | 5-Br | CH₃ | R | ii − | 7-CH₃ | CH₃ | R |
| Lix^{a} | | | | iix^{a} | | | |
| Ji − | 5-Br | CH₃ | S | ji − | 7-CH₃ | CH₃ | S |
| Jix^{a} | | | | jixª | | | |
| Ki − | 6-CH₃ | CH₃ | rac | ki − | 7-F | CH₃ | R |
| Kix^{a} | | | | kix^{a} | | | |
| Li − | 6-CH₃ | CH₃ | R | li − | 7-F | CH₃ | S |
| Lix^{a} | | | | lix^{a} | | | |
| Mv − | 6-OCH₃ | CH₃ | R | mi − | 7-Cl | CH₃ | R |
| Mix^{b} | | | | mix^{a} | | | |
| Nx − | 6- | CH₃ | R | nx − | 7-Cl | CH₃ | S |
| Nix^{c} | CH₂CH₃ | | | nixª | | | |
| Oi − | 6-F | CH₃ | rac | | | | |
| Oix^{a} | | | | | | | |
| Pi − | 6-F | CH₃ | R | | | | |
| Pix^{a} | | | | | | | |
| Qi − | 6-Cl | CH₃ | rac | | | | |
| Qix^{a} | | | | | | | |
| Ri − | 6-Cl | CH₃ | R | | | | |
| Rix^{a} | | | | | | | |
| Si − | 6-Br | CH₃ | R | | | | |
| Six^{d} | | | | | | | |
| Tv − | 7-CH₃ | CH₃ | rac | | | | |
| Tix^{b} | | | | | | | |
| Ui − | 7-F | CH₃ | rac | | | | |
| Uix^{a} | | | | | | | |
| Vi − | 7-Cl | CH₃ | rac | | | | |
| Vix^{a} | | | | | | | |
| Wi − | none | CH₂CH | rac | | | | |
| Wix^{b} | | CH₂ | | | | | |
| Xi − | none | CH₂CHC | rac | | | | |
| Xix^{b} | | (CH₃)₂ | | | | | |
| Yi − | none | CH₂CH₂ | rac | | | | |
| Yix^{b} | | OCH₃ | | | | | |
| Zi − | 5-CH₃ | CH₃ | rac | | | | |
| Zix^{a} | | | | | | | |

^{a}see Scheme-1.
^{b}see Scheme-2.
^{c}see Scheme-3.
^{d}see Scheme-5.

### General Procedure a for Esterification of Amino Acid (ii):

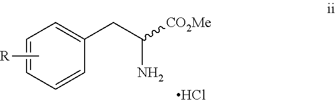

To a solution of amino acid i (1 equiv) in anhydrous methanol (0.2 M) was added thionyl chloride (1.1 equiv) at 0° C., and the reaction mixture was stirred at 50-70° C. for 16-18 h. After reaction completion, the crude was concentrated under reduced pressure to give ii as a hydrochloride salt (1:1).

General Procedure b1 for Protection of Amine (iii):

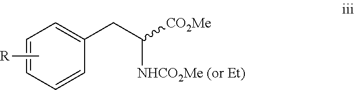

To a solution of ii (1 equiv) in dichloromethane (0.3 M) was added methyl chloroformate (1.05 equiv) and sodium bicarbonate (2.2 equiv) at 0° C., and the reaction mixture was stirred at room temperature for 16-18 h. After reaction completion, the crude was diluted with water, extracted with dichloromethane, then the organic layer was concentrated under reduced pressure to give iii.

General Procedure b2 for Protection of Amine (iii):

To a solution of ii (1 equiv) in dichloromethane (0.3 M) was added ethyl chloroformate (1.05 equiv) and pyridine (2.2 equiv) at 0° C., and the reaction mixture was stirred at 0° C. for 2-16 h. After reaction completion, the crude was diluted with brine, extracted with dichloromethane, then the organic layer was concentrated under reduced pressure to give iii.

### General Procedure c for Cyclization (iv):

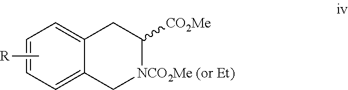

To a solution of (1 equiv) in a mixture of acetic acid and sulfuric acid (ratio: 3/1, the concentration of iii at 0.25 M) was added paraformaldehyde (1.1 equiv), and the reaction mixture was stirred at room temperature for 16-18 h. After reaction completion, the crude was neutralized with aqueous 6 N NaOH at ice bath, extracted with brine and dichloromethane, then the organic layer was concentrated under reduced pressure. The residue was purified by Isco Combi-Flash Companion column chromatography or flash column chromatography over silica gel to give iv.

### General Procedure d for Deprotection (v):

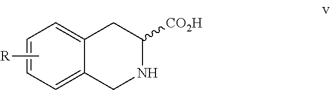

A mixture of iv (1 equiv) in aqueous 6-12 N hydrogen chloride (the concentration of iv at 0.1 M) was refluxed for 16-48 h. After reaction completion, the obtained precipitate was filtered, and the solid was washed by iced acetone, dried in vacuo to give v as a hydrochloride salt (1:1).

### General Procedure e1 for Methyl Esterification (vi):

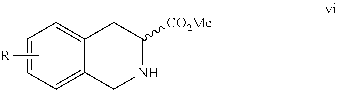

To a suspension of v (1 equiv) in methanol (0.1 M) was added sulfuric acid (2 equiv), and the suspension was refluxed for 16-18 h. After reaction completion, the crude was evaporated under reduced pressure, then the resulted residue was diluted with dichloromethane, washed with saturated aqueous NaHCO₃, and the organic layer was concentrated under reduced pressure. The crude was purified by Isco Combi-Flash Companion column chromatography or flash column chromatography over silica gel to give vi.

### General Procedure e2 for Methyl Esterification (vi):

To a solution of v (1 equiv) and triethylamine (1.1 equiv) in anhydrous methanol (the concentration of vi at 0.1 M) under an ice bath was added thionyl chloride (3.5 equiv) at 0° C., then the reaction mixture was stirred at 50° C. for 16-18 h. After reaction completion, the crude was diluted with dichloromethane, extracted with saturated aqueous NaHCO₃, and the organic layer was concentrated under reduced pressure. The crude was purified by Isco Combi-Flash Companion column chromatography or flash column chromatography over silica gel to give vi.

General Procedure f for Amidation of 3-Methyl Ester (vii):

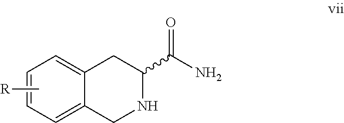

A solution of vi (1 equiv) in 28-30% aqueous ammonium hydroxide (0.1 M) was stirred at room temperature for 16-18 h. After reaction completion, the obtained precipitate was filtered, washed by iced water or iced dichloromethane, and dried in vacuo to give vii.

General Procedure g1 for Methylation of 2-Amino of Tetrahedroisoquinoline (viii):

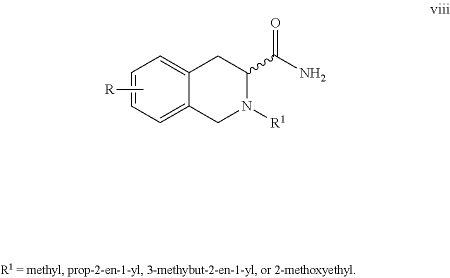

A suspension of vii (1 equiv) and 37% aqueous formaldehyde (1.5 equiv) in acetonitrile or tetrahydrofuran (the concentration of vii at 0.05 M) was stirred at room temperature for 0.5-1 h, then to the reaction mixture was added sodium triacetoxyborohydride (2 equiv) and acetic acid (1 equiv). The reaction mixture was stirred at room temperature for 1-16 h. After reaction completion, the crude mixture was diluted with dichloromethane or ethyl acetate, extracted with saturated aqueous NaHCO₃, and the organic layer was concentrated under reduced pressure. The crude was purified by Isco Combi-Flash Companion column chromatography or flash column chromatography over silica gel to give viii.

General Procedure g2 for Alkylation of 2-Amino of Tetrahydroisoquinoline (viii):

To a solution of vii (1 equiv) and potassium carbonate (1.2 equiv) in acetonitrile and tetrahydrofuran (ratio: 1/1, vii at 0.03 M) was added 3-bromopropene (1.05 equiv), and the reaction mixture was stirred at 60° C. for 4 h. After reaction completion, the crude was diluted with dichloromethane, extracted with brine, and the organic layer was concentrated under reduced pressure. The crude was purified by flash column chromatography over silica gel to give viii.

General Procedure g3 for Alkylation of 2-Amino of Tetrahedroisoquinoline (viii):

To a solution of vii (1 equiv) and potassium carbonate (1.2 equiv) in acetonitrile and tetrahydrofuran (ratio: 1/1, the concentration of vii at 0.05 M) was added 4-bromo-2-methyl-2-butene (1.05 equiv), and the reaction mixture was stirred at 60° C. for 4 h. After reaction completion, the crude mixture was diluted with dichloromethane, extracted with brine, and the organic layer was concentrated under reduced pressure. The crude was purified by flash column chromatography over silica gel to give viii.

General Procedure g4 for Alkylation of 2-Amino of Tetrahydroisoquinoline (viii):

To a solution of vii (1 equiv) in acetonitrile (the concentration of vii at 0.05 M) was added 2-bromoethyl methyl ether (1.5 equiv) and potassium carbonate (3 equiv), and the reaction mixture was refluxed for 19 h. After reaction completion, the crude mixture was diluted with dichloromethane, extracted with water, and the organic layer was concentrated under reduced pressure to give viii.

### General Procedure h for Reduction of 3-Carboxamide (ix):

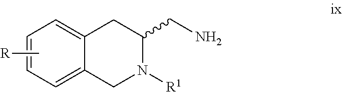

To a solution of viii (1 equiv) in anhydrous tetrahydrofuran (the concentration of viii at 0.05 M) was added lithium aluminium hydride (3 equiv), and the suspension was refluxed for 3-72 h. After reaction completion, the crude mixture was quenched with saturated aqueous Na₂SO₄, filtered through a short pad of celite, washed and the organic layer was concentrated under reduced pressure. The crude was purified by Isco Combi-Flash Companion column chromatography or flash column chromatography over silica gel to give ix.

### General Procedure i1 for Amide Bond Formation:

To a stirred solution of ix (1 equiv) in dichloromethane (the concentration of ix at 0.05-0.2 M) was added acid (0.9-1 equiv), 1-(3-dimethylaminopropyl)-3-ethylcarbodiimide hydrochloride (1.5 equiv), 1-hydroxybenzotriazole hydrate (1.5 equiv) and N,N-diisopropylethylamine (3.5 equiv) at room temperature, and the reaction mixture was stirred at room temperature for 16-18 h. After reaction completion, the crude mixture was diluted with dichloromethane, extracted with saturated brine, and the organic layer was concentrated under reduced pressure. The crude was purified by Isco Combi-Flash Companion column chromatography or flash column chromatography over silica gel to give the desired product.

### General Procedure i2 for Amide Bond Formation:

To a stirred solution of ix (1 equiv) and triethylamine (1.2 equiv) in dichloromethane (the concentration of ix at 0.1 M) was added acyl chloride (1.1 equiv), and the reaction mixture was stirred at room temperature for 1 h. After reaction completion, the crude mixture was diluted with dichloromethane, extracted with saturated brine, and the organic layer was concentrated under reduced pressure. The crude was purified by Isco Combi-Flash Companion column chromatography or flash column chromatography over silica gel to give the desired product.

### General Procedure i3 for Amide Bond Formation:

To a solution of ix (1 equiv), triethylamine (1.5 equiv) and propylphosphonic anhydride solution (50 wt. % in ethyl acetate) (1.5 equiv) in dichloromethane (the concentration of ix at 0.15 M) was added 3-dimethylaminobenzoic acid (1 equiv), and the reaction mixture was stirred at room temperature for 16 h. After reaction completion, the crude mixture was diluted with dichloromethane, extracted with water, and the organic layer was concentrated under reduced pressure. The crude was purified by flash column chromatography over silica gel to give the desired product.

### General Procedure j1 for Cyclization of Intermediate i (v):

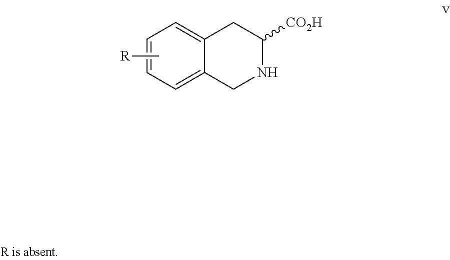

To a solution of amino acid i (1 equiv) in 38% aqueous hydrochloric acid (the concentration of i at 0.6-1.5 M) was added 37% aqueous formaldehyde (4-6 equiv), and the reaction mixture was refluxed for 7-24 h. After reaction completion, the obtained precipitate was filtered, washed with iced water and iced MeOH, and dried in vacuo to give v as a hydrochloride salt (1:1).

### General Procedure j2 for Cyclization of Intermediate i (v):

A mixture solution of amino acid i (1 equiv) in water (the concentration of i at 3.5 M) and 48% aqueous hydrobromic acid (70 equiv) was stirred at room temperature for 0.5 h, then to the reaction solution was added 37% aqueous formaldehyde (3 equiv). The reaction mixture was stirred at 65° C. for 5 h. After reaction completion, the solvent was evaporated under reduced pressure, then the obtained precipitate was triturated with ether, filtered, and dried in vacuo to give v.

### 2-Methyl-1,2,3,4-tetrahydroisoquinoline-3-carboxamide (Aviii)

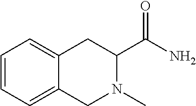

The compound was prepared by general procedure g1 with 1,2,3,4-tetrahydroisoquinoline-3-carboxamide Avii (1.5 g, 8.51 mmol), 37% aqueous formaldehyde (2.22 mL, 29.8 mmol) and sodium triacetoxyborohydride (7.22 g, 34.1 mmol) in acetonitrile (100 mL) and tetrahydrofuran (200 mL), stirred at room temperature for 17 h. After workup, the crude was purified by Isco Combi-Flash Companion column chromatography (0-5% methanol in CH₂Cl₂) to give the desired product 2-methyl-1,2,3,4-tetrahydroisoquinoline-3-carboxamide Aviii (1.39 g, 85.8%). ¹H NMR (300 MHz, CDCl₃) δ 7.24-7.12 (m, 3H), 7.12-7.05 (m, 1H), 6.94 (br s, 1H), 5.33 (br s, 1H), 3.87 (d, 1H), 3.70 (d, 1H), 3.28 (dd, 1H), 3.15-3.03 (m, 2H), 2.44 (s, 3H).

### (2-Methyl-1,2,3,4-tetrahydroisoquinolin-3-yl)methanamine (Aix)

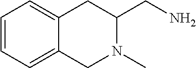

The compound was prepared by general procedure h with 2-methyl-1,2,3,4-tetrahydroisoquino-line-3-carboxamide Aviii (500 mg, 2.63 mmol) and lithium aluminium hydride (300 mg, 7.88 mmol) in tetrahydrofuran (30 mL) and CH₂Cl₂ (30 mL), stirred at 50° C. for 17 h. After workup, the crude was purified by Isco Combi-Flash Companion column chromatography (0-5% methanol with 1% ammonia hydroxide in CH₂Cl₂) to give the desired product (0.24 g, 52%). ¹H NMR (400 MHz, CDCl₃) δ 7.22-6.95 (m, 4H), 3.85 (d, 1H), 3.70 (d, 1H), 3.02-2.74 (m, 4H), 2.68 (dq, 1H), 2.41 (s, 3H).

### (3R)-1,2,3,4-Tetrahydroisoquinoline-3-carboxylic acid hydrochloride (1:1) (Bv)

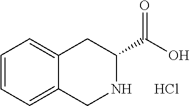 [SMILES: O=C(O)[C@H]1Cc2ccccc2CN1 ; Cl]

The compound was prepared by general procedure j1 with D-phenylalanine Bi (4 g, 24.2 mmol) and formaldehyde (10.9 mL, 145 mmol) in 38% aqueous hydrochloric acid (40 mL), refluxed for 7 h. Bv hydrochloride (1:1) was obtained as a white solid (3.84 g, 74%). ¹H NMR (CD₃OD, 400 MHz) δ 7.35-7.23 (m, 4H), 4.48-4.38 (m, 3H), 3.48 (dd, 1H), 3.24 (dd, 1H).

### Methyl (3R)-1,2,3,4-tetrahydroisoquinoline-3-carboxylate (Bvi)

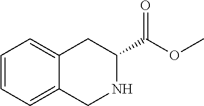

The compound was prepared by general procedure e2 with Bv hydrochloride (1:1) (0.26 g, 1.22 mmol), thionyl chloride (0.309 mL, 4.26 mmol) and triethylamine (0.187 mL, 1.34 mmol) in anhydrous methanol (12 mL), refluxed for 16 h. After workup, the crude was purified by flash column chromatography (1% methanol in dichloromethane) to give Bvi as a colorless liquid (0.209 g, 90%). ¹H NMR (CDCl₃, 400 MHz) δ 7.18-7.09 (m, 3H), 7.06-7.01 (m, 1H), 4.15 (d, 1H), 4.09 (d, 1H), 3.81-3.74 (m, 4H), 3.10 (dd, 1H), 2.97 (dd, 1H).

### (3R)-1,2,3,4-Tetrahydroisoquinoline-3-carboxamide (Bvii)

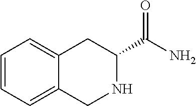 [SMILES: NC(=O)[C@H]1Cc2ccccc2CN1]

The compound was prepared by general procedure f with Bvi (3.06 g, 16 mmol) in 28-30% aqueous ammonium hydroxide (32 mL), stirred at room temperature for 16 h. Bvii was obtained as a white solid (2.58 g, 92%). ¹H NMR (CD₃OD, 400 MHz) δ 7.15-7.09 (m, 3H), 7.08-7.03 (m, 1H), 4.08-3.95 (m, 2H), 3.57 (dd, 1H), 3.04 (dd, 1H), 2.89 (dd, 1H).

### (3R)-2-Methyl-1,2,3,4-tetrahydroisoquinoline-3-carboxamide (Bviii)

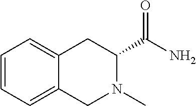

The compound was prepared by general procedure g1 with Bvii (0.118 g, 0.67 mmol), 37% aqueous formaldehyde (75.3 μL, 1.01 mmol), sodium triacetoxyborohydride (0.284 g, 1.34 mmol) and acetic acid (38.4 μL, 0.67 mmol) in acetonitrile (3.4 mL) and tetrahydrofuran (3.4 mL), stirred at room temperature for 16 h. Bviii was obtained as a white solid (0.122 g, 96%). ¹H NMR (CD₃OD, 400 MHz) δ 7.16-7.05 (m, 4H), 3.96 (d, 1H), 3.56 (d, 1H), 3.17-3.05 (m, 2H), 2.99 (dd, 1H), 2.44 (s, 3H).

### [(3R)-2-Methyl-1,2,3,4-tetrahydroisoquinolin-3-yl]methanamine (Bix)

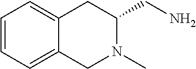

The compound was prepared by general procedure h with Bviii (0.8 g, 4.21 mmol) and lithium aluminium hydride (0.479 g, 12.6 mmol) in anhydrous tetrahydrofuran (21 mL), refluxed for 36 h. After workup, the crude was purified by flash column chromatography (SiO₂, dichloromethane/methanol/ammonium hydroxide solution=92/7.6/0.4) to give Bix as a yellow liquid (0.621 g, 84%). ¹H NMR (CDCl₃, 400 MHz) δ 7.16-7.06 (m, 3H), 7.04-6.99 (m, 1H), 3.84 (d, 1H), 3.70 (d, 1H), 2.84 (dd, 2H), 2.78 (d, 2H), 2.71-2.64 (m, 1H), 2.40 (s, 3H).

### Methyl (3S)-1,2,3,4-tetrahydroisoquinoline-3-carboxylate (Cvi)

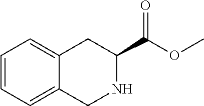

The compound was prepared by general procedure e1 with(S)-(−)-1,2,3,4-tetrahydroisoquinoline-3-carboxylic acid Cv (2 g, 11.3 mmol) and sulfuric acid (1 mL) in methanol (49.8 mL), refluxed for 16 h. Cvi was obtained as a yellow liquid (2.1 g, 97%). ¹H NMR (CDCl₃, 300 MHz) δ 7.18-7.08 (m, 3H), 7.06-7.00 (m, 1H), 4.15 (d, 1H), 4.08 (d, 1H), 3.78 (s, 3H), 3.76-3.71 (m, 1H), 3.10 (dd, 1H), 2.96 (dd, 1H).

### (3S)-1,2,3,4-Tetrahydroisoquinoline-3-carboxamide (Cvii)

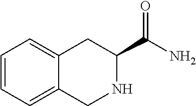 [SMILES: NC(=O)[C@@H]1Cc2ccccc2CN1]

The compound was prepared by general procedure f with Cvi (2.1 g, 11 mmol) in 28-30% aqueous ammonium hydroxide (30.5 mL), stirred at room temperature for 16 h. Cvii was obtained as a white solid (1.61 g, 83%). ¹H NMR (CD₃OD, 300 MHz) δ 7.16-7.10 (m, 3H), 7.09-7.02 (m, 1H), 4.06 (d, 1H), 3.98 (d, 1H), 3.56 (dd, 1H), 3.04 (dd, 1H), 2.88 (dd, 1H).

### (3S)-2-Methyl-1,2,3,4-tetrahydroisoquinoline-3-carboxamide (Cviii)

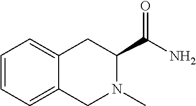

The compound was prepared by general procedure g1 with Cvii (1.6 g, 9.1 mmol), 37% aqueous formaldehyde (2.39 mL, 31.8 mmol), sodium triacetoxyborohydride (7.71 g, 36.4 mmol) and acetic acid (0.521 mL, 9.1 mmol) in acetonitrile (91 mL) and tetrahydrofuran (91 mL), stirred at room temperature for 16 h. Cviii was obtained as a white solid (1.73 g, quantitative yield). ¹H NMR (CD₃OD, 300 MHz) δ 7.17-7.04 (m, 4H), 3.99 (d, 1H), 3.59 (d, 1H), 3.22-3.13 (m, 1H), 3.08 (d, 1H), 3.00 (dd, 1H), 2.46 (s, 3H).

### [(3S)-2-Methyl-1,2,3,4-tetrahydroisoquinolin-3-yl]methanamine (Cix)

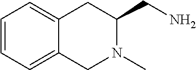

The compound was prepared by general procedure h with Cviii (0.8 g, 4.21 mmol) and lithium aluminium hydride (0.12 g, 3.15 mmol) in anhydrous tetrahydrofuran (19.7 mL), refluxed for 16 h. After workup, the crude was purified by flash column chromatography (SiO₂, dichloromethane/methanol/ammonium hydroxide solution=92/7.6/0.4) to give Cix as a yellow liquid (0.621 g, 84%). ¹H NMR (CDCl₃, 400 MHz) δ 7.16-7.06 (m, 3H), 7.04-6.99 (m, 1H), 3.84 (d, 1H), 3.70 (d, 1H), 2.84 (dd, 2H), 2.78 (d, 2H), 2.71-2.64 (m, 1H), 2.40 (s, 3H).

### Methyl (2R)-2-amino-3-(2-methylphenyl)propanoate hydrochloride (Dii)

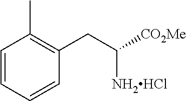

The compound was prepared by general procedure a with Di (4 g, 22.3 mmol) and thionyl chloride (3.23 mL, 44.6 mmol) in anhydrous methanol (60 mL), stirred at 50° C. for 17 h. Dii was obtained as a white solid (5.12 g, 99%). ¹H NMR (400 MHz, d₆-DMSO) δ 8.77 (br s, 3H), 7.23-7.07 (m, 4H), 4.09 (dd, 1H), 3.58 (s, 3H), 3.30-3.14 (m, 1H), 3.04 (dd, 1H), 2.28 (s, 3H).

### Methyl (2R)-2-((ethoxycarbonyl)amino)-3-(2-methylphenyl)propanoate (Diii)

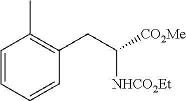

The compound was prepared by general procedure b2 with Dii (5.12 g, 22.9 mmol), ethyl chloroformate (3.38 mL, 24 mmol) and pyridine (4.06 mL, 50.4 mmol) in dichloromethane (110 mL), stirred at 0° C. for 2 h. After workup, the crude was purified by Isco Combi-Flash Companion column chromatography (0-20% ethyl acetate in n-hexane) to give Diii (5.56 g, 92%). ¹H NMR (400 MHz, d₆-DMSO) δ 7.69 (d, 1H), 7.20-7.04 (m, 4H), 4.27-4.15 (m, 1H), 3.98-3.86 (m, 2H), 3.61 (s, 3H), 3.04 (dd, 1H), 2.84 (dd, 1H), 2.28 (s, 3H), 1.11 (t, 3H).

### 2-Ethyl 3-methyl (3R)-5-methyl-3,4-dihydroisoquinoline-2,3(1H)-dicarboxylate (Div)

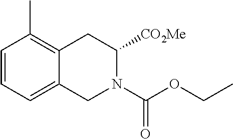 [SMILES: CCOC(=O)N1Cc2cccc(C)c2C[C@@H]1C(=O)OC]

The compound was prepared by general procedure c with Diii (5.56 g, 21 mmol) and paraformaldehyde (0.69 g, 23.1 mmol) in a mixture of acetic acid (60 mL) and sulfuric acid (20 mL), stirred at room temperature for 17 h. After workup, the crude was purified by Isco Combi-Flash Companion column chromatography (0-30% ethyl acetate in n-hexane) to give Div as a colorless liquid (4.57 g, 79%). ¹H NMR (400 MHz, d₆-DMSO) δ 7.19-6.95 (m, 3H), 5.05-4.93 (m, 1H), 4.61 (dd, 1H), 4.41 (dd, 1H), 4.21-4.04 (m, 2H), 3.60-3.52 (m, 3H), 3.16 (dd, 1H), 3.00 (td, 1H), 2.22 (s, 3H), 1.24 (t, 1.5H), 1.16 (t, 1.5H).

### (3R)-5-Methyl-1,2,3,4-tetrahydroisoquinoline-3-carboxylic acid hydrochloride (1:1) (Dv)

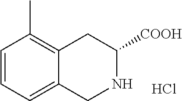

The compound was prepared by general procedure d with Div (4.57 g, 16.5 mmol) in aqueous 12 N hydrogen chloride (100 mL), refluxed for 17 h. Dv hydrochloride was obtained as a white solid (3.44 g, 92%). ¹H NMR (400 MHz, d₆-DMSO) δ 9.97 (br s, 1H), 9.79 (br s, 1H), 7.22-7.11 (m, 2H), 7.09 (dd, 1H), 4.49-4.34 (m, 1H), 4.34-4.24 (m, 2H), 3.21 (dd, 1H), 2.91 (dd, 1H), 2.23 (s, 3H).

### Methyl (3R)-5-methyl-1,2,3,4-tetrahydroisoquinoline-3-carboxylate (Dvi)

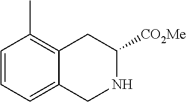

The compound was prepared by general procedure e2 with (3R)-5-methyl-1,2,3,4-tetrahydroisoqui-noline-3-carboxylic acid Dv hydrochloride (1:1) (3.44 g, 15.1 mmol) and thionyl chloride (3.84 mL, 52.9 mmol) in anhydrous methanol (60 mL), stirred at 50° C. for 17 h. After workup, the crude was concentrated to Dvi (3.05 g, 98%). ¹H NMR (400 MHz, d₆-DMSO) δ 7.05-6.92 (m, 2H), 6.86 (d, 1H), 4.00-3.82 (m, 2H), 3.72-3.64 (m, 4H), 2.82 (dd, 1H), 2.61 (dd, 1H), 2.16 (s, 3H).

### (3R)-5-Methyl-1,2,3,4-tetrahydroisoquinoline-3-carboxamide (Dvii)

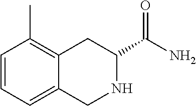

The compound was prepared by general procedure f with Dvi (2.1 g, 11 mmol) in 28-30% aqueous ammonium hydroxide (140 mL), stirred at room temperature for 17 h, to give Dvii (2.33 g, 82%). ¹H NMR (400 MHz, d₆-DMSO) δ 7.32 (br s, 1H), 7.10 (br s, 1H), 7.03-6.94 (m, 2H), 6.85 (dd, 1H), 3.92-3.80 (m, 2H), 3.38-3.28 (m overlapped with H₂O, 1H), 2.78 (dd, 1H), 2.55-2.44 (m overlapped with d₆-DMSO, 1H), 2.16 (s, 3H).

### (3R)-2,5-Dimethyl-1,2,3,4-tetrahydroisoquinoline-3-carboxamide (Dviii)

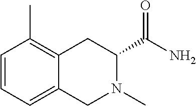

The compound was prepared by general procedure g1 with Dvii (2.33 g, 12.2 mmol), 37% aqueous formaldehyde (1.37 mL, 18.4 mmol), sodium triacetoxyborohydride (5.19 g, 24.5 mmol) and acetic acid (0.7 mL, 12.2 mmol) in acetonitrile (60 mL) and tetrahydrofuran (60 mL), stirred at room temperature for 2 h. After workup, the crude was purified by Isco Combi-Flash Companion column chromatography (0-5% methanol in CH₂Cl₂) to give Dviii (1.7 g, 68%). ¹H NMR (400 MHz, d₆-DMSO) δ 7.42 (br s, 1H), 7.11 (br s, 1H), 7.06-6.95 (m, 2H), 6.88 (d, 1H), 3.83 (d, 1H), 3.45 (d, 1H), 3.03 (dd, 1H), 2.83-2.65 (m, 2H), 2.29 (s, 3H), 2.16 (s, 3H).

### ((3R)-2,5-Dimethyl-1,2,3,4-tetrahydroisoquinolin-3-yl)methanamine (Dix)

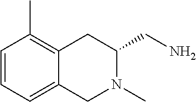

The compound was prepared by general procedure h with Dviii (0.8 g, 4.21 mmol) and 1M lithium aluminium hydride in THF (17 mL, 17 mmol) in anhydrous tetrahydrofuran (50 mL) and CH₂Cl₂ (50 mL), stirred at 60° C. for 17 h. After workup, the crude was purified by Isco Combi-Flash Companion column chromatography (0-5% methanol with 1% ammonia hydroxide in CH₂Cl₂) to give Dix (0.73 g, 68%). ¹H NMR (400 MHz, d₆-DMSO) δ 7.04-6.90 (m, 2H), 6.84 (d, 1H), 3.71 (d, 1H), 3.48 (d, 1H), 2.72 (dd, 1H), 2.63 (dd, 1H), 2.56 (d, 2H), 2.48-2.39 (m, 1H), 2.26 (s, 3H), 2.16 (s, 3H).

### Methyl (2S)-2-amino-3-(2-methylphenyl)propanoate hydrochloride (1:1) (Eii)

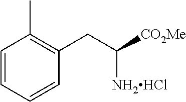

The compound was prepared by general procedure a with Ei (4 g, 22.3 mmol) and thionyl chloride (3.23 mL, 44.6 mmol) in anhydrous methanol (60 mL), stirred at 50° C. for 17 h. The desired product methyl (2S)-2-amino-3-(2-methylphenyl)propanoate hydrochloride (1:1) Eii was obtained as a white solid (5.1 g, 99%). ¹H NMR (400 MHz, d₆-DMSO) δ 8.76 (br s, 3H), 7.24-7.08 (m, 4H), 4.10 (br s, 1H), 3.58 (s, 3H), 3.21 (dd, 1H), 3.04 (dd, 1H), 2.28 (s, 3H).

### Methyl (2S)-2-((ethoxycarbonyl)amino)-3-(2-methylphenyl)propanoate (Eiii)

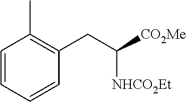

The compound was prepared by general procedure b2 with Eii hydrochloride (5.12 g, 22.9 mmol), ethyl chloroformate (3.27 mL, 23.3 mmol) and pyridine (3.93 mL, 48.8 mmol) in dichloromethane (110 mL), stirred at 0° C. for 2 h. After workup, the crude was purified by Isco Combi-Flash Companion column chromatography (0-20% ethyl acetate in n-hexane) to give Eiii (5.8 g, 99%). ¹H NMR (400 MHz, d₆-DMSO) δ 7.69 (d, 1H), 7.20-7.05 (m, 4H), 4.21 (ddd, 1H), 3.99-3.86 (m, 2H), 3.61 (s, 3H), 3.04 (dd, 1H), 2.84 (dd, 1H), 2.28 (s, 3H), 1.11 (t, 3H).

### 2-Ethyl 3-methyl (3S)-5-methyl-3,4-dihydroisoquinoline-2,3(1H)-dicarboxylate (Eiv)

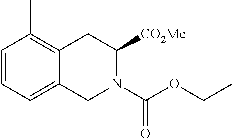 [SMILES: CCOC(=O)N1Cc2cccc(C)c2C[C@H]1C(=O)OC]

The compound was prepared by general procedure c with Eiii (5.8 g, 21.9 mmol) and paraformaldehyde (0.72 g, 24 mmol) in a mixture of acetic acid (60 mL) and sulfuric acid (20 mL), stirred at room temperature for 17 h. After workup, the crude was purified by Isco Combi-Flash Companion column chromatography (0-30% ethyl acetate in n-hexane) to give Eiv as a colorless liquid (5.1 g, 84%). ¹H NMR (400 MHz, d₆-DMSO) δ 7.15-6.99 (m, 3H), 4.97 (ddd, 1H), 4.61 (dd, 1H), 4.41 (dd, 1H), 4.19-4.06 (m, 2H), 3.57 (s, 1.5H), 3.55 (s, 1.5H), 3.16 (t, 1H), 3.00 (td, 1H), 2.22 (s, 1.5H), 2.22 (s, 1.5H), 1.24 (t, 1.5H), 1.16 (t, 1.5H).

### (3S)-5-Methyl-1,2,3,4-tetrahydroisoquinoline-3-carboxylic acid hydrochloride (1:1) (Ev)

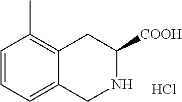

The compound was prepared by general procedure d Eiv (5.1 g, 18.4 mmol) in aqueous 12 N hydrogen chloride (100 mL), refluxed for 17 h. Ev was obtained as a white solid (3.44 g, 82%). ¹H NMR (400 MHz, d₆-DMSO) δ 9.86 (br s, 2H), 7.20-7.13 (m, 2H), 7.09 (dd, 1H), 4.39 (dd, 1H), 4.30 (s, 2H), 3.20 (dd, 1H), 2.91 (dd, 1H), 2.23 (s, 3H).

### Methyl (3S)-5-methyl-1,2,3,4-tetrahydroisoquinoline-3-carboxylate (Evi)

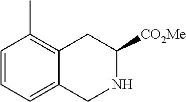

The compound was prepared by general procedure e2 with Ev hydrochloride (1:1) (3.44 g, 15.1 mmol) and thionyl chloride (3.84 mL, 52.9 mmol) in anhydrous methanol (60 mL), stirred at 50° C. for 17 h. After workup, the crude was concentrated to give Evi (3.1 g, 99%). ¹H NMR (400 MHz, d₆-DMSO) δ 7.07-6.94 (m, 2H), 6.86 (d, 1H), 3.96-3.82 (m, 2H), 3.73-3.62 (m, 4H), 2.82 (dd, 1H), 2.62 (dd, 1H), 2.17 (s, 3H).

### (3S)-5-Methyl-1,2,3,4-tetrahydroisoquinoline-3-carboxamide (Evii)

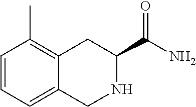

The compound was prepared by general procedure f with Evi (3.1 g, 15.1 mmol) in 28-30% aqueous ammonium hydroxide (150 mL), stirred at room temperature for 17 h, to give Evii (2.4 g, 84%). ¹H NMR (600 MHz, d₆-DMSO) δ 7.40 (s, 1H), 7.18 (s, 1H), 7.06-6.95 (m, 2H), 6.88 (d, 1H), 3.92 (s, 2H), 3.50-3.38 (m, 1H), 2.84 (d, 1H), 2.59-2.51 (m, 1H), 2.18 (s, 3H).

### (3S)-2,5-Dimethyl-1,2,3,4-tetrahydroisoquinoline-3-carboxamide (Eviii)

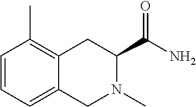

The compound was prepared by general procedure g1 with Evii (2.4 g, 12.6 mmol), 37% aqueous formaldehyde (1.41 mL, 18.9 mmol), sodium triacetoxyborohydride (5.35 g, 25.2 mmol) and acetic acid (0.7 mL, 12.6 mmol) in acetonitrile (60 mL) and tetrahydrofuran (60 mL), stirred at room temperature for 2 h. After workup, the crude was purified by Isco Combi-Flash Companion column chromatography (0-5% methanol in CH₂Cl₂) to give Eviii (2.08 g, 81%). ¹H NMR (400 MHz, d₆-DMSO) δ 7.42 (br s, 1H), 7.11 (br s, 1H), 7.06-6.96 (m, 2H), 6.88 (d, 1H), 3.83 (d, 1H).

### 3.45 (d, 1H), 3.03 (dd, 1H), 2.84-2.65 (m, 2H), 2.29 (s, 3H), 2.16 (s, 3H). ((3S)-2,5-Dimethyl-1,2,3,4-tetrahydroisoquinolin-3-yl)methanamine (Eix)

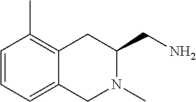

The compound was prepared by general procedure h with Eviii (1.1 g, 5.38 mmol) and 1M lithium aluminium hydride in THF (16.2 mL, 16.2 mmol) in anhydrous tetrahydrofuran (50 mL) and CH₂Cl₂ (50 mL), stirred at 60° C. for 17 h. After workup, the crude was purified by Isco Combi-Flash Companion column chromatography (0-5% methanol with 1% ammonia hydroxide in CH₂Cl₂) to give Eix (0.44 g, 43%). ¹H NMR (600 MHz, d₆-DMSO) δ 7.02-6.93 (m, 2H), 6.85 (d, 1H), 3.72 (d, 1H), 3.50 (d, 1H), 2.74 (dd, 1H), 2.65 (dd, 1H), 2.62-2.52 (m, 2H), 2.49-2.43 (m, 1H), 2.27 (s, 3H), 2.17 (s, 3H).

### Methyl 2-amino-3-(2-chlorophenyl)propanoate hydrochloride (1:1) (Fii)

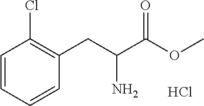

The compound was prepared by general procedure a with 2-amino-3-(2-chlorophenyl) propanoic acid Fi (5 g, 25 mmol) and thionyl chloride (2 mL, 27.6 mmol) in anhydrous methanol (63 mL), refluxed for 16 h. Fii hydrochloride (1:1) was obtained as a white solid (6.23 g, quantitative yield). ¹H NMR (CD₃OD, 400 MHz) δ 7.49-7.45 (m, 1H), 7.38-7.32 (m, 3H), 4.33 (t, 1H), 3.76 (s, 3H), 3.44 (dd, 1H), 3.27 (dd, 1H).

### Methyl 3-(2-chlorophenyl)-2-[(methoxycarbonyl)amino]propanoate (Fiii)

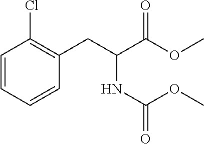 [SMILES: COC(=O)NC(Cc1ccccc1Cl)C(=O)OC]

The compound was prepared by general procedure b1 with Fii hydrochloride (1:1) (6.23 g, 24.9 mmol), methyl chloroformate (2.02 mL, 26.2 mmol) and sodium bicarbonate (4.61 g, 54.8 mmol) in dichloromethane (83.1 mL), stirred at room temperature for 16 h. Fiii was obtained as a yellow liquid (6.77 g, quantitative yield). ¹H NMR (CDCl₃, 400 MHz) δ 7.38-7.33 (m, 1H), 7.22-7.17 (m, 3H), 5.26 (br d, 1H), 4.67 (q, 1H), 3.71 (s, 3H), 3.63 (s, 3H), 3.29 (dd, 1H), 3.14 (dd, 1H).

### Dimethyl 5-chloro-3,4-dihydroisoquinoline-2,3(1H)-dicarboxylate (Fiv)

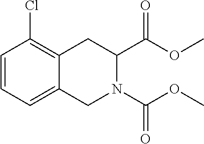 [SMILES: COC(=O)C1Cc2c(Cl)cccc2CN1C(=O)OC]

The compound was prepared by general procedure c with Fiii (6.95 g, 25.6 mmol) and paraformaldehyde (1.15 g, 38.4 mmol) in a mixture of acetic acid (38.4 mL) and sulfuric acid (25.6 mL), stirred at room temperature for 16 h. Fiv was obtained as a colorless liquid (3.93 g, 54%). ¹H NMR (CDCl₃, 400 MHz) δ 7.24 (d, 1H), 7.13 (t, 1H), 7.02 (dd, 1H), 5.23 (dd, 0.6H), 5.03 (dd, 0.4H), 4.79 (dd, 1H), 4.54 (dd, 1H), 3.80 (s, 2H), 3.76 (s, 1H), 3.64 (s, 3H), 3.55 (t, 1H), 3.11-2.99 (m, 1H).

### 5-Chloro-1,2,3,4-tetrahydroisoquinoline-3-carboxylic acid hydrochloride (1:1) (Fv)

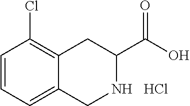 [SMILES: O=C(O)C1Cc2c(Cl)cccc2CN1 ; Cl]

The compound was prepared by general procedure d with Fiv (3.93 g, 13.8 mmol) in aqueous 6 N hydrogen chloride (46.2 mL), refluxed for 48 h. Fv hydrochloride (1:1) was obtained as a white solid (2.71 g, 79%). ¹H NMR (CD₃OD, 400 MHz) δ 7.45 (d, 1H), 7.33 (t, 1H), 7.25 (d, 1H), 4.55-4.43 (m, 3H), 3.57 (dd, 1H), 3.10 (dd, 1H).

### Methyl 5-chloro-1,2,3,4-tetrahydroisoquinoline-3-carboxylate (Fvi)

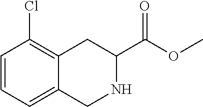

The compound was prepared by general procedure e2 with Fv hydrochloride (1:1) (2.71 g, 10.9 mmol), thionyl chloride (2.77 mL, 38.3 mmol) and triethylamine (1.68 mL, 12 mmol) in anhydrous methanol (36.4 mL), refluxed for 16 h. Fvi was obtained as a yellow liquid (2.47 g, quantitative yield). ¹H NMR (CDCl₃, 400 MHz) δ 7.23 (d, 1H), 7.09 (t, 1H), 6.95 (d, 1H), 4.15-4.03 (m, 2H), 3.80 (s, 3H), 3.74 (dd, 1H), 3.19 (dd, 1H), 2.83 (dd, 1H).

### 5-Chloro-1,2,3,4-tetrahydroisoquinoline-3-carboxamide (Fvii)

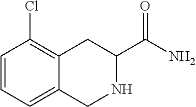 [SMILES: NC(=O)C1Cc2c(Cl)cccc2CN1]

The compound was prepared by general procedure f with Fvi (2.64 g, 11.7 mmol) in 28-30% aqueous ammonium hydroxide (38.9 mL), stirred at room temperature for 16 h. The desired product 5-chloro-1,2,3,4-tetrahydroisoquinoline-3-carboxamide Fvii was obtained as a white solid (2 g, 81%). ¹H NMR (CD₃OD, 400 MHz) δ 7.24 (d, 1H), 7.14 (t, 1H), 7.04 (d, 1H), 4.07 (d, 1H), 3.99 (d, 1H), 3.58 (dd, 1H), 3.17 (dd, 1H), 2.74 (dd, 1H).

### 5-Chloro-2-methyl-1,2,3,4-tetrahydroisoquinoline-3-carboxamide (Fviii)

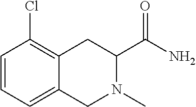

The compound was prepared by general procedure g1 with Fvii (0.5 g, 2.37 mmol), 37% aqueous formaldehyde (0.267 mL, 3.56 mmol), sodium triacetoxyborohydride (1.01 g, 4.75 mmol) and acetic acid (0.136 mL, 2.37 mmol) in acetonitrile (11.9 mL) and tetrahydrofuran (11.9 mL), stirred at room temperature for 16 h. Fviii was obtained as a white solid (0.506 g, 95%). ¹H NMR (CD₃OD, 400 MHz) δ 7.25 (d, 1H), 7.15 (t, 1H), 7.05 (d, 1H), 3.97 (d, 1H), 3.59 (d, 1H), 3.20 (dd, 1H), 3.10-2.97 (m, 2H), 2.44 (s, 3H).

### (5-Chloro-2-methyl-1,2,3,4-tetrahydroisoquinolin-3-yl)methanamine (Fix)

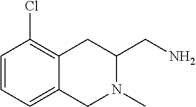

The compound was prepared by general procedure h with Fviii (0.506 g, 2.25 mmol) and lithium aluminium hydride (0.256 g, 6.75 mmol) in anhydrous tetrahydrofuran (22.5 mL), refluxed for 72 h. After workup, the crude was purified by flash column chromatography (SiO₂, dichloromethane/methanol/ammonium hydroxide solution=92/7.6/0.4) to give Fix as a yellow liquid (0.303 g, 64%). ¹H NMR (CDCl₃, 400 MHz) δ 7.21 (d, 1H), 7.07 (t, 1H), 6.94 (d, 1H), 3.83 (d, 1H), 3.70 (d, 1H), 2.88 (dd, 2H), 2.83-2.76 (m, 1H), 2.71-2.64 (m, 2H), 2.37 (s, 3H).

### Methyl (2R)-2-amino-3-(2-chlorophenyl)propanoate hydrochloride (1:1) (Gii)

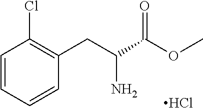

The compound was prepared by general procedure a with 2-chloro-D-phenylalanine Gi (0.8 g, 4.01 mmol) and thionyl chloride (0.32 mL, 4.41 mmol) in anhydrous methanol (10 mL), refluxed for 16 h. Gii hydrochloride (1:1) was obtained as a white solid (0.856 g, quantitative yield). ¹H NMR (CD₃OD, 400 MHz) δ 7.49-7.45 (m, 1H), 7.38-7.32 (m, 3H), 4.33 (t, 1H), 3.76 (s, 3H), 3.44 (dd, 1H), 3.26 (dd, 1H).

### Methyl (2R)-3-(2-chlorophenyl)-2-[(methoxycarbonyl)amino]propanoate (Giii)

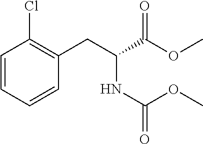 [SMILES: COC(=O)N[C@H](Cc1ccccc1Cl)C(=O)OC]

The compound was prepared by general procedure b1 with Gii hydrochloride (1:1) (1.24 g, 4.95 mmol), methyl chloroformate (4.02 mL, 5.2 mmol) and sodium bicarbonate (0.832 g, 9.9 mmol) in dichloromethane (49.5 mL), stirred at room temperature for 16 h. Giii was obtained as a yellow liquid (1.21 g, 90%). ¹H NMR (CDCl₃, 400 MHz) δ 7.37-7.33 (m, 1H), 7.21-7.15 (m, 3H), 5.28 (br d, 1H), 4.67 (q, 1H), 3.71 (s, 3H), 3.62 (s, 3H), 3.29 (dd, 1H), 3.14 (dd, 1H).

### Dimethyl (3R)-5-chloro-3,4-dihydroisoquinoline-2,3(1H)-dicarboxylate (Giv)

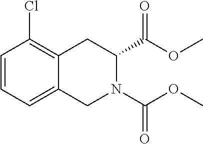 [SMILES: COC(=O)[C@H]1Cc2c(Cl)cccc2CN1C(=O)OC]

The compound was prepared by general procedure c with Giii (1.21 g, 4.45 mmol) and paraformaldehyde (0.2 g, 6.67 mmol) in a mixture of acetic acid (6.7 mL) and sulfuric acid (4.4 mL), stirred at room temperature for 16 h. Giv was obtained as a colorless liquid (0.756 g, 60%). ¹H NMR (CDCl₃, 400 MHz) δ 7.24 (d, 1H), 7.13 (t, 1H), 7.02 (dd, 1H), 5.23 (dd, 0.6H), 5.03 (dd, 0.4H), 4.79 (dd, 1H), 4.54 (dd, 1H), 3.80 (s, 2H), 3.75 (s, 1H), 3.64 (s, 3H), 3.55 (t, 1H), 3.10-2.99 (m, 1H).

### (3R)-5-Chloro-1,2,3,4-tetrahydroisoquinoline-3-carboxylic acid hydrochloride (1:1) (Gv)

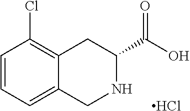 [SMILES: O=C(O)[C@H]1Cc2c(Cl)cccc2CN1 ; Cl]

The compound was prepared by general procedure d with Giv (0.756 g, 2.67 mmol) in aqueous 6 N hydrogen chloride (26.7 mL), refluxed for 48 h. Gv hydrochloride (1:1) was obtained as a white solid (0.624 g, 94%). ¹H NMR (CD₃OD, 400 MHz) δ 7.45 (d, 1H), 7.33 (t, 1H), 7.25 (d, 1H), 4.55-4.43 (m, 3H), 3.57 (dd, 1H), 3.10 (dd, 1H).

### Methyl (3R)-5-chloro-1,2,3,4-tetrahydroisoquinoline-3-carboxylate (Gvi)

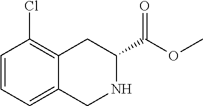

The compound was prepared by general procedure e2 with Gv hydrochloride (1:1) (0.524 g, 2.11 mmol), thionyl chloride (0.537 mL, 7.4 mmol) and triethylamine (0.442 mL, 3.17 mmol) in anhydrous methanol (21 mL), refluxed for 16 h. Gvi was obtained as a yellow liquid (0.476 g, quantitative yield). ¹H NMR (CDCl₃, 400 MHz) δ 7.22 (d, 1H), 7.09 (t, 1H), 6.95 (d, 1H), 4.12 (d, 1H), 4.05 (d, 1H), 3.80 (s, 3H), 3.74 (dd, 1H), 3.19 (dd, 1H), 2.82 (dd, 1H).

### (3R)-5-Chloro-1,2,3,4-tetrahydroisoquinoline-3-carboxamide (Gvii)

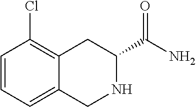 [SMILES: NC(=O)[C@H]1Cc2c(Cl)cccc2CN1]

The compound was prepared by general procedure f with Gvi (0.476 g, 2.11 mmol) in 28-30% aqueous ammonium hydroxide (21 mL), stirred at room temperature for 16 h. Gvii was obtained as a white solid (0.452 g, 85%). ¹H NMR (CD₃OD, 400 MHz) δ 7.24 (d, 1H), 7.14 (t, 1H), 7.04 (d, 1H), 4.07 (d, 1H), 3.99 (d, 1H), 3.58 (dd, 1H), 3.17 (dd, 1H), 2.74 (dd, 1H).

### (3R)-5-Chloro-2-methyl-1,2,3,4-tetrahydroisoquinoline-3-carboxamide (Gviii)

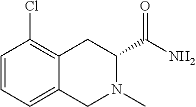

The compound was prepared by general procedure g1 with Gvii (0.452 g, 2.14 mmol), 37% aqueous formaldehyde (0.563 mL, 7.51 mmol) and sodium triacetoxyborohydride (1.82 g, 8.58 mmol) in acetonitrile (42.9 mL), stirred at room temperature for 16 h. After workup, the crude was purified by flash column chromatography (SiO₂, dichloromethane/methanol=95/5) to give Gviii as a white solid (0.342 g, 71%). ¹H NMR (CD₃OD, 400 MHz) δ 7.24 (d, 1H), 7.14 (t, 1H), 7.04 (d, 1H), 3.97 (d, 1H), 3.58 (d, 1H), 3.19 (dd, 1H), 3.09-2.95 (m, 2H), 2.44 (s, 3H).

### [(3R)-5-Chloro-2-methyl-1,2,3,4-tetrahydroisoquinolin-3-yl]methanamine (Gix)

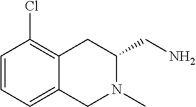

The compound was prepared by general procedure h with Gviii (0.342 g, 1.52 mmol) and lithium aluminium hydride (0.173 g, 4.56 mmol) in anhydrous tetrahydrofuran (20 mL), refluxed for 72 h. After workup, the crude was purified by flash column chromatography (SiO₂, dichloromethane/methanol/ammonium hydroxide solution=92/7.6/0.4) to give Gix as a yellow liquid (0.179 g, 56%). ¹H NMR (CDCl₃, 400 MHz) δ 7.18 (d, 1H), 7.04 (t, 1H), 6.91 (d, 1H), 3.80 (d, 1H), 3.66 (d, 1H), 2.86-2.71 (m, 3H), 2.69-2.59 (m, 2H), 2.33 (s, 3H).

### Methyl 2-amino-3-(2-bromophenyl)propanoate hydrochloride (1:1) (Hii)

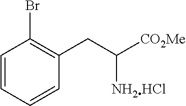

The compound was prepared by general procedure a with 2-amino-3-(4-methylphenyl)-propanoic acid Hi (0.3 g, 1.23 mmol) and thionyl chloride (0.178 mL, 2.46 mmol) in anhydrous methanol (10 mL), stirred at 50° C. for 17 h. Hii hydrochloride (1:1) was obtained as a white solid (0.28 g, 78%). ¹H NMR (400 MHz, d₆-DMSO) δ 8.60 (br s, 2H), 7.63 (dd, 1H), 7.41-7.34 (m, 2H), 7.30-7.20 (m, 1H), 4.19 (br s, 1H), 3.61 (s, 3H), 3.23 (d, 2H).

### Methyl 3-(2-bromophenyl)-2-((ethoxycarbonyl)amino)propanoate (Hiii)

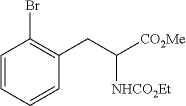

The compound was prepared by general procedure b2 with Hii hydrochloride (1:1) (0.28 g, 0.956 mmol), ethyl chloroformate (0.141 mL, 1 mmol) and pyridine (0.169 mL, 2.1 mmol) in dichloromethane (5 mL), stirred at 0° C. for 2 h. After workup, the crude was purified by Isco Combi-Flash Companion column chromatography (0-20% ethyl acetate in n-hexane) to give Hiii (0.23 g, 73%). ¹H NMR (400 MHz, d₆-DMSO) δ 7.71 (d, 1H), 7.59 (d, 1H), 7.41-7.27 (m, 2H), 7.24-7.14 (m, 1H), 4.41-4.25 (m, 1H), 3.98-3.83 (m, 2H), 3.62 (s, 3H), 3.19 (dd, 1H), 2.94 (dd, 1H), 1.10 (t, 3H).

### 2-Ethyl 3-methyl 5-bromo-3,4-dihydroisoquinoline-2,3(1H)-dicarboxylate (Hiv)

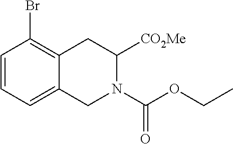 [SMILES: CCOC(=O)N1Cc2cccc(Br)c2CC1C(=O)OC]

The compound was prepared by general procedure c with Hiii (0.23 g, 0.697 mmol) and paraformaldehyde (23 g, 0.766 mmol) in a mixture of acetic acid (3 mL) and sulfuric acid (1 mL), stirred at room temperature for 17 h. After workup, the crude was purified by Isco Combi-Flash Companion column chromatography (0-30% ethyl acetate in n-hexane) to give Hiv as a colorless liquid (95.6 mg, 40%). ¹H NMR (400 MHz, d₆-DMSO) δ 7.51 (d, 1H), 7.35-7.22 (m, 1H), 7.16 (t, 1H), 5.15-4.94 (m, 1H), 4.67 (dd, 1H), 4.45 (dd, 1H), 4.23-4.06 (m, 2H), 3.57 (s, 3H), 3.43-3.27 (m overlapped with H₂O, 1H), 3.19-2.99 (m, 1H), 1.18 (t, 1.5H), 1.17 (t, 1.5H).

### 5-Bromo-1,2,3,4-tetrahydroisoquinoline-3-carboxylic acid hydrochloride (1:1) (Hv)

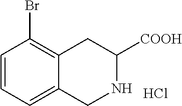 [SMILES: O=C(O)C1Cc2c(Br)cccc2CN1 ; Cl]

The compound was prepared by general procedure d with Hiv (2.48 g, 7.25 mmol) in aqueous 12 N hydrogen chloride (70 mL), refluxed for 17 h. Hv hydrochloride (1:1) was obtained as a white solid (1.79 g, 84%). ¹H NMR (400 MHz, d₆-DMSO) δ 9.93 (br s, 2H), 7.61 (dd, 1H), 7.32 (d, 1H), 7.24 (t, 1H), 4.46 (dd, 1H), 4.38 (d, 1H), 4.34 (d, 1H), 3.27 (dd, 1H), 2.96 (dd, 1H).

### Methyl 5-bromo-1,2,3,4-tetrahydroisoquinoline-3-carboxylate (Hvi)

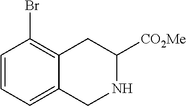 [SMILES: COC(=O)C1Cc2c(Br)cccc2CN1]

The compound was prepared by general procedure e2 with Hv hydrochloride (1:1) (1.79 g, 6.12 mmol), thionyl chloride (1.55 mL, 21.4 mmol) and triethylamine (0.94 mL, 6.73 mmol) in anhydrous methanol (25 mL), stirred at 50° C. for 17 h. After workup, the crude mixture was concentrated to give Hvi (1.63 g, 98%). ¹H NMR (400 MHz, d₆-DMSO) δ 7.43 (t, 1H), 7.14-7.03 (m, 2H), 3.93 (d, 1H), 3.86 (d, 1H), 3.72 (dd, 1H), 3.68 (s, 3H), 2.90 (dd, 1H), 2.67 (dd, 1H).

### 5-Bromo-1,2,3,4-tetrahydroisoquinoline-3-carboxamide (Hvii)

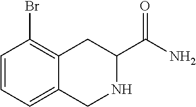 [SMILES: NC(=O)C1Cc2c(Br)cccc2CN1]

The compound was prepared by general procedure f with Hvi (1.63 g, 6.03 mmol) in 28-30% aqueous ammonium hydroxide (60 mL), stirred at room temperature for 17 h, to give Hvii (1.2 g, 78%). ¹H NMR (400 MHz, d₆-DMSO) δ 7.43 (t, 1H), 7.37 (br s, 1H), 7.14 (br s, 1H), 7.10-7.05 (m, 2H), 3.91 (d, 1H), 3.85 (d, 1H), 3.39 (dd, 1H), 2.87 (dd, 1H), 2.56 (dd, 1H).

### 5-Bromo-2-methyl-1,2,3,4-tetrahydroisoquinoline-3-carboxamide (Hviii)

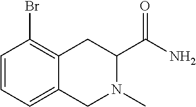

The compound was prepared by general procedure g1 with Hvii (1.2 g, 4.7 mmol), 37% aqueous formaldehyde (0.53 mL, 7.06 mmol), sodium triacetoxyborohydride (1.99 g, 9.41 mmol) and acetic acid (0.270 mL, 4.7 mmol) in acetonitrile (25 mL) and tetrahydrofuran (25 mL), stirred at room temperature for 2 h. After workup, the crude was purified by Isco Combi-Flash Companion column chromatography (0-5% methanol in CH₂Cl₂) to give Hviii (0.34 g, 27%). ¹H NMR (400 MHz, d₆-DMSO) δ 7.49-7.41 (m, 2H), 7.15 (br s, 1H), 7.11-7.07 (m, 2H), 3.87 (d, 1H), 3.53 (d, 1H), 3.15 (dd, 1H), 2.85-2.76 (m, 2H), 2.30 (s, 3H).

### (5-Bromo-2-methyl-1,2,3,4-tetrahydroisoquinolin-3-yl)methanamine (Hix)

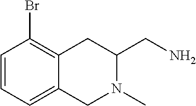

The compound was prepared by general procedure h with Hviii (0.57 g, 2.11 mmol) and 1M lithium aluminium hydride in THF (6.4 mL, 6.35 mmol) in tetrahydrofuran (30 mL) and CH₂Cl₂ (30 mL), stirred at 60° C. for 17 h. After workup, the crude was purified by Isco Combi-Flash Companion column chromatography (0-5% methanol with 1% ammonia hydroxide in CH₂Cl₂) to give Hix (0.32 g, 59%). ¹H NMR (400 MHz, d₆-DMSO) δ 7.46-7.38 (m, 1H), 7.08-7.05 (m, 2H), 3.76 (d, 1H), 3.52 (d, 1H), 2.73 (dd, 1H), 2.68-2.60 (m, 3H), 2.53-2.43 (m overlapped with d₆-DMSO, 1H), 2.26 (s, 3H).

### Methyl (2R)-2-amino-3-(2-bromophenyl)propanoate hydrochloride (1:1) (Iii)

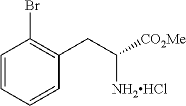

The compound was prepared by general procedure a with Ii (1.5 g, 6.15 mmol) and thionyl chloride (0.89 mL, 12.3 mmol) in anhydrous methanol (5 mL), stirred at 50° C. for 17 h. Iii hydrochloride (1:1) was obtained as a white solid (1.8 g, 99%). ¹H NMR (400 MHz, d₆-DMSO) δ 8.73 (br s, 3H), 7.64 (dd, 1H), 7.45-7.33 (m, 2H), 7.29-7.20 (m, 1H), 4.17 (t, 1H), 3.61 (s, 3H), 3.33-3.18 (m, 2H).

### Methyl (2R)-3-(2-bromophenyl)-2-((ethoxycarbonyl)amino)propanoate (Iiii)

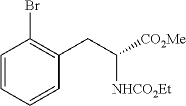

The compound was prepared by general procedure b2 with Iii hydrochloride (1:1) (1.8 g, 6.13 mmol), ethyl chloroformate (0.91 mL, 6.45 mmol) and pyridine (1.09 mL, 13.5 mmol) in dichloromethane (30 mL), stirred at 0° C. for 2 h. After workup, the crude was purified by Isco Combi-Flash Companion column chromatography (0-20% ethyl acetate in n-hexane) to give Iiii (2 g, 99%). ¹H NMR (400 MHz, d₆-DMSO) δ 7.70 (d, 1H), 7.58 (dd, 1H), 7.41-7.25 (m, 2H), 7.17 (t, 1H), 4.41-4.25 (m, 1H), 3.98-3.82 (m, 2H), 3.61 (s, 3H), 3.18 (dd, 1H), 2.93 (dd, 1H), 1.09 (t, 3H).

### 2-Ethyl 3-methyl (3R)-5-bromo-3,4-dihydroisoquinoline-2,3(1H)-dicarboxylate (Iiv)

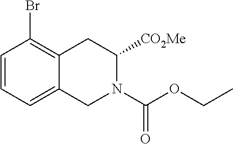 [SMILES: CCOC(=O)N1Cc2cccc(Br)c2C[C@@H]1C(=O)OC]

The compound was prepared by general procedure c with Iiii (2 g, 6.06 mmol) and paraformaldehyde (0.2 g, 6.66 mmol) in a mixture of acetic acid (18 mL) and sulfuric acid (6 mL), stirred at room temperature for 17 h. After workup, the crude was purified by Isco Combi-Flash Companion column chromatography (0-30% ethyl acetate in n-hexane) to give liv as a colorless liquid (0.890 g, 43%). ¹H NMR (400 MHz, d₆-DMSO) δ 7.51 (d, 1H), 7.35-7.23 (m, 1H), 7.17 (t, 1H), 5.16-4.96 (m, 1H), 4.67 (dd, 1H), 4.45 (dd, 1H), 4.22-4.04 (m, 2H), 3.57 (d, 3H), 3.43-3.29 (m overlapped with H₂O, 1H), 3.15-2.96 (m, 1H), 1.24 (t, 1H), 1.17 (t, 2H).

### (3R)-5-Bromo-1,2,3,4-tetrahydroisoquinoline-3-carboxylic acid hydrochloride (1:1) (Iv)

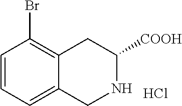 [SMILES: O=C(O)[C@H]1Cc2c(Br)cccc2CN1 ; Cl]

The compound was prepared by general procedure d with liv (0.89 g, 2.6 mmol) in aqueous 6 N hydrogen chloride (26 mL), refluxed for 17 h. Iv hydrochloride (1:1) was obtained as a white solid (0.360 g, 47%). ¹H NMR (400 MHz, d₆-DMSO) δ 9.88 (br s, 2H), 7.63 (dd, 1H), 7.33 (dd, 1H), 7.26 (t, 1H), 4.48 (dd, 1H), 4.40 (d, 1H), 4.33 (d, 1H), 3.28 (dd, 1H), 2.96 (dd, 1H).

### Methyl (3R)-5-Bromo-1,2,3,4-tetrahydroisoquinoline-3-carboxylate (Ivi)

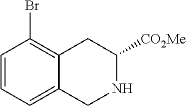 [SMILES: COC(=O)[C@H]1Cc2c(Br)cccc2CN1]

The compound was prepared by general procedure e2 with Iv hydrochloride (1:1) (0.75 g, 2.56 mmol), thionyl chloride (0.65 mL, 8.96 mmol) and triethylamine (0.390 mL, 2.82 mmol) in anhydrous methanol (30 mL), stirred at 50° C. for 17 h. After workup, the crude mixture was concentrated to give Ivi (0.66 g, 95%). ¹H NMR (400 MHz, d₆-DMSO) δ 7.49-7.39 (m, 1H), 7.12-7.06 (m, 2H), 3.93 (d, 1H), 3.87 (d, 1H), 3.73 (dd, 1H), 3.69 (s, 3H), 2.91 (dd, 1H), 2.68 (dd, 1H).

### (3R)-5-Bromo-1,2,3,4-tetrahydroisoquinoline-3-carboxamide (Ivii)

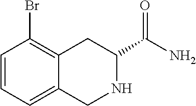

The compound was prepared by general procedure f with Ivi (0.66 g, 2.44 mmol) in 28-30% aqueous ammonium hydroxide (25 mL), stirred at room temperature for 17 h, to give Ivii (0.42 g, 64%). ¹H NMR (400 MHz, d₆-DMSO) δ 7.40 (t, 1H), 7.07-7.02 (m, 2H), 3.91 (d, 1H), 3.84 (d, 1H), 3.42-3.38 (m, 1H), 2.89 (dd, 1H), 2.57 (dd, 1H).

### (3R)-5-Bromo-2-methyl-1,2,3,4-tetrahydroisoquinoline-3-carboxamide (Iviii)

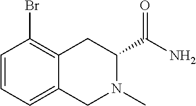

The compound was prepared by general procedure g1 with Ivii (0.42 g, 1.65 mmol), 37% aqueous formaldehyde (0.2 mL, 2.47 mmol), sodium triacetoxyborohydride (0.7 g, 3.29 mmol) and acetic acid (90 μL, 1.65 mmol) in acetonitrile (8 mL) and tetrahydrofuran (8 mL), stirred at room temperature for 2 h. After workup, the crude was purified by Isco Combi-Flash Companion column chromatography (0-5% methanol in CH₂Cl₂) to give Iviii (0.37 g, 84%). ¹H NMR (400 MHz, d₆-DMSO) δ 7.51-7.42 (m, 2H), 7.16 (s, 1H), 7.12-7.08 (m, 2H), 3.88 (d, 1H), 3.54 (d, 1H), 3.16 (t, 1H), 2.84-2.75 (m, 2H), 2.31 (s, 3H).

### ((3R)-5-Bromo-2-methyl-1,2,3,4-tetrahydroisoquinolin-3-yl)methanamine (Iix)

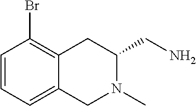

The compound was prepared by general procedure h with Iviii (0.2 g, 0.743 mmol) and lithium aluminium hydride (84.6 mg, 2.23 mmol) in tetrahydrofuran (15 mL) and CH₂Cl₂ (15 mL), stirred at 60° C. for 17 h. After workup, the crude was purified by Isco Combi-Flash Companion column chromatography (0-5% methanol with 1% ammonia hydroxide in CH₂Cl₂) to give Iix (61 mg, 32%). ¹H NMR (400 MHz, d₆-DMSO) δ 7.44 (t, 1H), 7.14-7.04 (m, 2H), 3.77 (d, 1H), 3.55 (d, 1H), 2.82-2.59 (m, 3H), 2.61-2.51 (m, 2H), 2.30 (s, 3H).

### Methyl (2S)-2-amino-3-(2-bromophenyl)propanoate hydrochloride (1:1) (Jii)

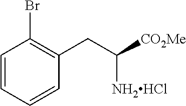

The compound was prepared by general procedure a with (2S)-2-amino-3-(2-bromophenyl) propanoic acid Ji (5 g, 20.5 mmol) and thionyl chloride (2.97 mL, 41 mmol) in anhydrous methanol (100 mL), stirred at 50° C. for 17 h. Jii hydrochloride (1:1) was obtained as a white solid (6 g, 99%). ¹H NMR (400 MHz, d₆-DMSO) δ 8.75 (s, 3H), 7.64 (dd, 1H), 7.48-7.33 (m, 2H), 7.31-7.21 (m, 1H), 4.16 (dd, 1H), 3.61 (s, 3H), 3.34-3.18 (m, 2H).

### Methyl (2S)-3-(2-bromophenyl)-2-((ethoxycarbonyl)amino)propanoate (Jiii)

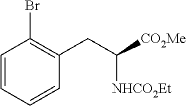

The compound was prepared by general procedure b2 with Jii hydrochloride (1:1) (6 g, 20.5 mmol), ethyl chloroformate (3.02 mL, 21.5 mmol) and pyridine (3.63 mL, 45.1 mmol) in dichloromethane (102 mL), stirred at 0° C. for 2 h. After workup, the crude was purified by Isco Combi-Flash Companion column chromatography (0-20% ethyl acetate in n-hexane) to give Jiii (6.76 g, 99%). ¹H NMR (400 MHz, d₆-DMSO) δ 7.71 (d, 1H), 7.59 (dd, 1H), 7.39-7.26 (m, 2H), 7.23-7.15 (m, 1H), 4.41-4.26 (m, 1H), 3.98-3.83 (m, 2H), 3.62 (s, 3H), 3.19 (dd, 1H), 2.94 (dd, 1H), 1.10 (t, 3H).

### 2-Ethyl 3-methyl (3S)-5-bromo-3,4-dihydroisoquinoline-2,3(1H)-dicarboxylate (Jiv)

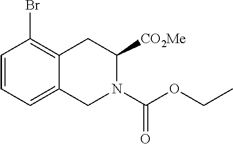 [SMILES: CCOC(=O)N1Cc2cccc(Br)c2C[C@H]1C(=O)OC]

The compound was prepared by general procedure c with Jiii (6.76 g, 20.5 mmol) and paraformaldehyde (0.680 g, 22.5 mmol) in a mixture of acetic acid (60 mL) and sulfuric acid (20 mL), stirred at room temperature for 17 h. After workup, the crude was purified by Isco Combi-Flash Companion column chromatography (0-30% ethyl acetate in n-hexane) to give Jiv as a colorless liquid (4.65 g, 66%). ¹H NMR (400 MHz, d₆-DMSO) δ 7.51 (dd, 1H), 7.33-7.24 (m, 1H), 7.17 (t, 1H), 5.18-4.98 (m, 1H), 4.66 (dd, 1H), 4.46 (dd, 1H), 4.21-4.07 (m, 1H), 3.58 (d, 3H), 3.46-3.26 (m, 2H), 3.09 (td, 1H), 1.25 (t, 1.5H), 1.18 (t, 1.5H).

### (3S)-5-Bromo-1,2,3,4-tetrahydroisoquinoline-3-carboxylic acid hydrochloride (1:1) (Jv)

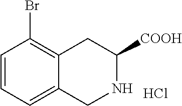 [SMILES: O=C(O)[C@@H]1Cc2c(Br)cccc2CN1 ; Cl]

The compound was prepared by general procedure d with Jiv (4.65 g, 13.6 mmol) in aqueous 6 N hydrogen chloride (100 mL), refluxed for 17 h. Jv hydrochloride (1:1) was obtained as a white solid (3.35 g, 56%). ¹H NMR (400 MHz, d₆-DMSO) δ 10.06 (br s, 2H), 7.63 (d, 1H), 7.34 (d, 1H), 7.26 (t, 1H), 4.48 (dd, 1H), 4.39 (d, 1H), 4.33 (d, 1H), 3.29 (dd, 1H), 2.99 (dd, 1H).

### Methyl (3S)-5-Bromo-1,2,3,4-tetrahydroisoquinoline-3-carboxylate (Jvi)

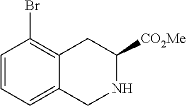 [SMILES: COC(=O)[C@@H]1Cc2c(Br)cccc2CN1]

The compound was prepared by general procedure e2 with Jv hydrochloride (1:1) (3.35 g, 11.5 mmol), thionyl chloride (2.9 mL, 40.1 mmol) and triethylamine (1.76 mL, 12.6 mmol) in anhydrous methanol (45.8 mL), stirred at 50° C. for 17 h. After workup, the crude mixture was concentrated to give Jvi (3.09 g, 99%). ¹H NMR (400 MHz, d₆-DMSO) δ 7.49-7.40 (m, 1H), 7.13-7.05 (m, 2H), 3.94 (d, 1H), 3.87 (d, 1H), 3.73 (dd, 1H), 3.69 (s, 3H), 2.91 (dd, 1H), 2.68 (dd, 1H).

### (3S)-5-Bromo-1,2,3,4-tetrahydroisoquinoline-3-carboxamide (Jvii)

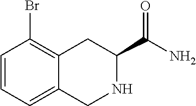 [SMILES: NC(=O)[C@@H]1Cc2c(Br)cccc2CN1]

The compound was prepared by general procedure f with Jvi (3.09 g, 11.4 mmol) in 28-30% aqueous ammonium hydroxide (110 mL), stirred at room temperature for 17 h, to give Jvii (2.5 g, 86%). ¹H NMR (400 MHz, d₆-DMSO) δ 7.43 (t, 1H), 7.38 (br s, 1H), 7.15 (br s, 1H), 7.12-7.04 (m, 2H), 3.92 (d, 1H), 3.85 (d, 1H), 3.40 (dd, 1H), 2.87 (dd, 1H), 2.57 (dd, 1H).

### (3S)-5-Bromo-2-methyl-1,2,3,4-tetrahydroisoquinoline-3-carboxamide (Jviii)

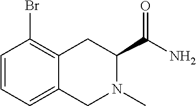

The compound was prepared by general procedure g1 with Jvii (2.5 g, 9.8 mmol), 37% aqueous formaldehyde (1.09 mL, 14.7 mmol), sodium triacetoxyborohydride (4.15 g, 19.6 mmol) and acetic acid (0.56 mL, 9.8 mmol) in acetonitrile (50 mL) and tetrahydrofuran (50 mL), stirred at room temperature for 2 h. After workup, the crude was purified by Isco Combi-Flash Companion column chromatography (0-5% methanol in CH₂Cl₂) to give Jviii (2.17 g, 82%). ¹H NMR (400 MHz, d₆-DMSO) δ 7.49-7.42 (m, 2H), 7.16 (br s, 1H), 7.12-7.07 (m, 2H), 3.88 (d, 1H), 3.54 (d, 1H), 3.16 (dd, 1H), 2.86-2.75 (m, 2H), 2.31 (s, 3H).

### ((3S)-5-Bromo-2-methyl-1,2,3,4-tetrahydroisoquinolin-3-yl)methanamine (Jix)

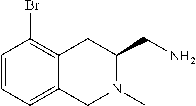

The compound was prepared by general procedure h with Jviii (1 g, 3.72 mmol) and 1M lithium aluminium hydride in THF (11.1 mL, 11.1 mmol) in tetrahydrofuran (50 mL) and CH₂Cl₂ (50 mL), stirred at 60° C. for 17 h. After workup, the crude was purified by Isco Combi-Flash Companion column chromatography (0-3% methanol with 1% ammonia hydroxide in CH₂Cl₂) to give Jix (0.519 g, 55%). ¹H NMR (400 MHz, d₆-DMSO) δ 7.46-7.39 (m, 1H), 7.11-7.04 (m, 2H), 3.76 (d, 1H), 3.53 (d, 1H), 2.74 (dd, 1H), 2.69-2.60 (m, 3H), 2.54-2.47 (m overlapped with d₆-DMSO, 1H), 2.27 (s, 3H).

### Methyl 2-amino-3-(3-methylphenyl)propanoate hydrochloride (1:1) (Kii)

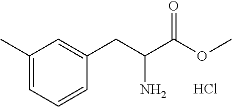

The compound was prepared by general procedure a with 2-amino-3-(3-methylphenyl)-propanoic acid Ki (5 g, 27.9 mmol) and thionyl chloride (2.23 mL, 30.7 mmol) in anhydrous methanol (69.7 mL), refluxed for 16 h. Kii hydrochloride (1:1) was obtained as a white solid (6.41 g, quantitative yield). ¹H NMR (CD₃OD, 400 MHz) δ 7.25 (t, 1H), 7.14 (d, 1H), 7.09 (s, 1H), 7.04 (d, 1H), 4.30 (t, 1H), 3.81 (s, 3H), 3.23 (dd, 1H), 3.12 (dd, 1H), 2.35 (s, 3H).

### Methyl 2-[(methoxycarbonyl)amino]-3-(3-methylphenyl)propanoate (Kiii)

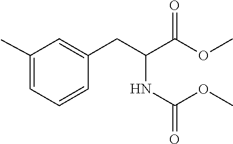

The compound was prepared by general procedure b1 with Kii hydrochloride (1:1) (6.61 g, 28.8 mmol), methyl chloroformate (2.33 mL, 30.2 mmol) and sodium bicarbonate (5.32 g, 63.3 mmol) in dichloromethane (71.9 mL), stirred at room temperature for 16 h. The desired product Kiii was obtained as a yellow liquid (7.03 g, 97%). ¹H NMR (CDCl₃, 400 MHz) δ 7.17 (t, 1H), 7.05 (d, 1H), 6.94-6.89 (m, 2H), 5.15 (br d, 1H), 4.62 (br q, 1H), 3.71 (s, 3H), 3.66 (s, 3H), 3.11-2.98 (m, 2H), 2.32 (s, 3H).

### Dimethyl 6-methyl-3,4-dihydroisoquinoline-2,3(1H)-dicarboxylate (Kiv)

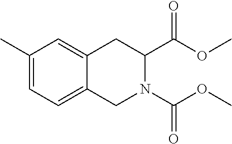

The compound was prepared by general procedure c with Kiii (7.03 g, 28 mmol) and paraformaldehyde (1.26 g, 42 mmol) in a mixture of acetic acid (35 mL) and sulfuric acid (2.98 mL), stirred at room temperature for 17 h. Kiv was obtained as a yellow liquid (6.45 g, 88%). ¹H NMR (CDCl₃, 400 MHz) δ 7.11-6.93 (m, 3H), 5.24-5.11 (m, 0.6H), 5.03-4.91 (m, 0.4H), 4.71 (dd, 1H), 4.55-4.38 (m, 1H), 3.85-3.72 (m, 3H), 3.62 (s, 3H), 3.30-3.08 (m, 2H), 2.34-2.21 (m, 3H).

### 6-Methyl-1,2,3,4-tetrahydroisoquinoline-3-carboxylic acid hydrochloride (1:1) (Kv)

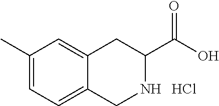

The compound was prepared by general procedure d with Kiv (6.45 g, 24.5 mmol) in aqueous 12 N hydrogen chloride (61.2 mL), refluxed for 16 h. The desired product Kv hydrochloride (1:1) was obtained as a white solid (3.91 g, 70%). ¹H NMR (CD₃OD, 400 MHz) δ 7.16-7.10 (m, 3H), 4.48-4.27 (m, 3H), 3.43 (dd, 1H), 3.20 (dd, 1H), 2.33 (s, 3H).

### Methyl 6-methyl-1,2,3,4-tetrahydroisoquinoline-3-carboxylate (Kvi)

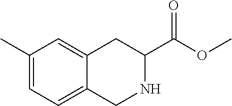

The compound was prepared by general procedure e2 with Kv hydrochloride (1:1) (3.91 g, 17.2 mmol), thionyl chloride (4.36 mL, 60.2 mmol) and triethylamine (2.64 mL, 18.9 mmol) in anhydrous methanol (57.3 mL), refluxed for 16 h. After workup, the crude was purified by flash column chromatography (SiO₂, dichloromethane/methanol=99.5/0.5) to give Kvi as a yellow liquid (0.327 g, 9%). ¹H NMR (CDCl₃, 400 MHz) δ 6.98-6.90 (m, 3H), 4.09 (d, 1H), 4.03 (d, 1H), 3.77 (s, 3H), 3.72 (dd, 1H), 3.03 (dd, 1H), 2.91 (dd, 1H), 2.30 (s, 3H).

### 6-Methyl-1,2,3,4-tetrahydroisoquinoline-3-carboxamide (Kvii)

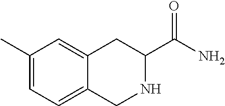

The compound was prepared by general procedure f with Kvi (0.327 g, 1.59 mmol) in 28-30% aqueous ammonium hydroxide (4 mL), stirred at room temperature for 16 h. Kvii was obtained as a white solid (0.262 g, 87%). ¹H NMR (CD₃OD, 400 MHz) δ 6.97-6.91 (m, 3H), 4.01 (d, 1H), 3.95 (d, 1H), 3.54 (dd, 1H), 2.99 (dd, 1H), 2.85 (dd, 1H), 2.27 (s, 3H).

### 2,6-Dimethyl-1,2,3,4-tetrahydroisoquinoline-3-carboxamide (Kviii)

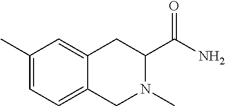

The compound was prepared by general procedure g1 with Kvii (0.262 g, 1.38 mmol), 37% aqueous formaldehyde (0.155 mL, 2.07 mmol), sodium triacetoxyborohydride (0.584 g, 2.76 mmol) and acetic acid (78.9 μL, 1.38 mmol) in acetonitrile (6.90 mL) and tetrahydrofuran (6.90 mL), stirred at room temperature for 16 h. Kviii was obtained as a white solid (0.255 g, 91%). ¹H NMR (CD₃OD, 400 MHz) δ 6.96-6.92 (m, 3H), 3.91 (d, 1H), 3.52 (d, 1H), 3.12 (dd, 1H), 3.05 (dd, 1H), 2.94 (dd, 1H), 2.43 (s, 3H), 2.27 (s, 3H).

### (2,6-Dimethyl-1,2,3,4-tetrahydroisoquinolin-3-yl)methanamine (Kix)

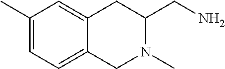

The compound was prepared by general procedure h with Kviii (0.255 g, 0.125 mmol) and lithium aluminium hydride (0.142 g, 3.75 mmol) in tetrahydrofuran (12.5 mL), refluxed for 48 h. After workup, the crude was purified by flash column chromatography (SiO₂, dichloromethane/methanol/ammonium hydroxide solution=92/7.6/0.4) to give Kix as a yellow liquid (0.179 g, 75%). ¹H NMR (CDCl₃, 400 MHz) δ 6.96-6.89 (m, 3H), 3.82 (d, 1H), 3.67 (d, 1H), 2.89-2.79 (m, 2H), 2.76-2.64 (m, 3H), 2.39 (s, 3H), 2.29 (s, 3H).

### Methyl (2R)-2-amino-3-(3-methylphenyl)propanoate hydrochloride (1:1) (Lii)

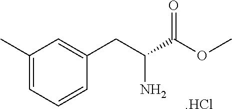

The compound was prepared by general procedure a with Li (5 g, 27.9 mmol) and thionyl chloride (2.23 mL, 30.7 mmol) in anhydrous methanol (40 mL), refluxed for 16 h. The desired product Lii hydrochloride (1:1) was obtained as a white solid (6.41 g, quantitative yield). ¹H NMR (CD₃OD, 400 MHz) δ 7.26 (t, 1H), 7.15 (d, 1H), 7.08 (s, 1H), 7.04 (d, 1H), 4.31 (dd, 1H), 3.81 (s, 3H), 3.23 (dd, 1H), 3.11 (dd, 1H), 2.35 (s, 3H).

### Methyl (2R)-2-[(methoxycarbonyl)amino]-3-(3-methylphenyl)propanoate (Liii)

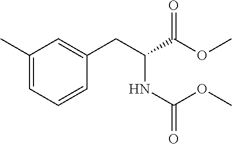

The compound was prepared by general procedure b1 with Lii hydrochloride (1:1) (7.45 g, 32.5 mmol), methyl chloroformate (2.63 mL, 34.1 mmol) and sodium bicarbonate (6 g, 71.4 mmol) in dichloromethane (81.1 mL), stirred at room temperature for 16 h. The desired product Liii was obtained as a yellow liquid (7.39 g, 91%). ¹H NMR (CDCl₃, 400 MHz) δ 7.18 (t, 1H), 7.06 (d, 1H), 6.94-6.89 (m, 2H), 5.12 (br d, 1H), 4.62 (br q, 1H), 3.72 (s, 3H), 3.66 (s, 3H), 3.12-2.98 (m, 2H), 2.32 (s, 3H).

### Dimethyl (3R)-6-methyl-3,4-dihydroisoquinoline-2,3(1H)-dicarboxylate (Liv)

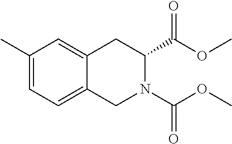

The compound was prepared by general procedure c with Liii (7.39 g, 29.4 mmol) and paraformaldehyde (1.33 g, 44.1 mmol) in a mixture of acetic acid (36.8 mL) and sulfuric acid (3.14 mL), stirred at room temperature for 16 h. Liv was obtained as a yellow liquid (5.2 g, 67%). ¹H NMR (CDCl₃, 400 MHz) δ 7.11-6.93 (m, 3H), 5.23-5.11 (m, 0.6H), 5.03-4.90 (m, 0.4H), 4.78-4.63 (m, 1H), 4.55-4.37 (m, 1H), 3.83-3.71 (m, 3H), 3.62 (s, 3H), 3.30-3.08 (m, 2H), 2.33-2.21 (m, 3H).

### (3R)-6-Methyl-1,2,3,4-tetrahydroisoquinoline-3-carboxylic acid hydrochloride (1:1) (Lv)

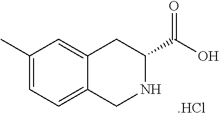

The compound was prepared by general procedure d with Liv (5.2 g, 19.8 mmol) in aqueous 6 N hydrogen chloride (65.8 mL), refluxed for 48 h. The suspension was filtered at 60° C., the precipitate was dried under high vacuum to afford Lv hydrochloride (1:1) as a white solid (1.68 g, 37%). ¹H NMR (CD₃OD, 400 MHz) δ 7.14-7.10 (m, 3H), 4.45-4.34 (m, 3H), 3.43 (dd, 1H), 3.19 (dd, 1H), 2.33 (s, 3H).

### Methyl (3R)-6-methyl-1,2,3,4-tetrahydroisoquinoline-3-carboxylate (Lvi)

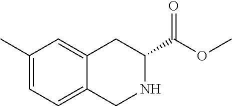

The compound was prepared by general procedure e2 with Lv hydrochloride (1:1) (1.68 g, 7.38 mmol), thionyl chloride (1.87 mL, 25.8 mmol) and triethylamine (1.13 mL, 8.12 mmol) in anhydrous methanol (24.6 mL), stirred at room temperature for 16 h. After workup, the crude was purified by flash column chromatography (SiO₂, dichloromethane/methanol=99/1) to give Lvi as a yellow liquid (1.04 g, 69%). ¹H NMR (CDCl₃, 400 MHz) δ 6.99-6.91 (m, 3H), 4.10 (d, 1H), 4.04 (d, 1H), 3.78 (s, 3H), 3.73 (dd, 1H), 3.04 (dd, 1H), 2.92 (dd, 1H), 2.30 (s, 3H).

### (3R)-6-Methyl-1,2,3,4-tetrahydroisoquinoline-3-carboxamide (Lvii)

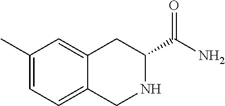

The compound was prepared by general procedure f with Lvi (1.04 g, 5.09 mmol) in 28-30% aqueous ammonium hydroxide (50.9 mL), stirred at room temperature for 16 h. Lvii was obtained as a white solid (0.830 g, 86%). ¹H NMR (CD₃OD, 400 MHz) δ 6.97-6.91 (m, 3H), 4.01 (d, 1H), 3.94 (d, 1H), 3.54 (dd, 1H), 2.99 (dd, 1H), 2.85 (dd, 1H), 2.27 (s, 3H).

### (3R)-2,6-Dimethyl-1,2,3,4-tetrahydroisoquinoline-3-carboxamide (Lviii)

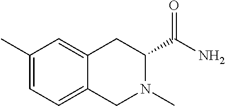

The compound was prepared by general procedure g1 with Lvii (0.83 g, 4.36 mmol), 37% aqueous formaldehyde (0.49 mL, 6.54 mmol), sodium triacetoxyborohydride (1.85 g, 8.72 mmol) and acetic acid (0.25 mL, 4.36 mmol) in acetonitrile (21.8 mL) and tetrahydrofuran (21.8 mL), stirred at room temperature for 16 h. The desired product Lviii was obtained as a white solid (0.884 g, 99%). ¹H NMR (CDCl₃, 400 MHz) δ 7.05-6.94 (m, 4H), 5.57 (br s, 1H), 3.84 (d, 1H), 3.68 (d, 1H), 3.28 (t, 1H), 3.04 (d, 2H), 2.44 (s, 3H), 2.31 (s, 3H). [(3R)-2,6-Dimethyl-1,2,3,4-tetrahydroisoquinolin-3-yl]methanamine (Lix)

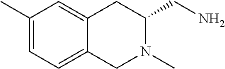

The compound was prepared by general procedure h with Lviii (0.748 g, 3.66 mmol) and lithium aluminium hydride (0.417 g, 11 mmol) in anhydrous tetrahydrofuran (36.6 mL), refluxed for 48 h. After workup, the crude was purified by flash column chromatography (SiO₂, dichloromethane/methanol/ammonium hydroxide solution=92/7.6/0.4) to give the desired product Lix as a yellow liquid (0.430 g, 62%). ¹H NMR (CDCl₃, 400 MHz) δ 6.97-6.88 (m, 3H), 3.81 (d, 1H), 3.67 (d, 1H), 2.89-2.79 (m, 2H), 2.76-2.64 (m, 3H), 2.39 (s, 3H), 2.29 (s, 3H).

### (3R)-6-Methoxy-1,2,3,4-tetrahydroisoquinoline-3-carboxylic acid hydrochloride (1:1) (Mv)

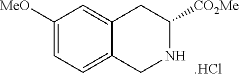 [SMILES: COC(=O)[C@H]1Cc2cc(OC)ccc2CN1 ; ".HCl"]

The compound was prepared by general procedure j1 with (2R)-2-amino-3-(3-methoxyphenyl) propanoic acid Mi (2 g, 10.2 mmol) and formaldehyde (3.33 mL, 41 mmol) in aqueous 6 M hydrogen chloride (6.80 mL), stirred at room temperature for 18 h. Mv hydrochloride (1:1) was obtained as a white solid (7.27 g, 29%). ¹H NMR (300 MHz, d₆-DMSO) δ 9.81 (br s, 2H), 7.17 (d, 1H), 6.89-6.81 (m, 2H), 4.37 (dd, 1H), 4.32-4.14 (m, 2H), 3.74 (s, 3H), 3.28 (dd overlapped with H₂O, 1H), 3.10 (dd, 1H).

### Methyl (3R)-6-methoxy-1,2,3,4-tetrahydroisoquinoline-3-carboxylate hydrochloride (1:1) (Mvi)

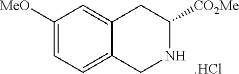 [SMILES: COC(=O)[C@H]1Cc2cc(OC)ccc2CN1 ; ".HCl"]

The compound was prepared by general procedure e2 with Mv hydrochloride (1:1) (727 mg, 2.98 mmol) and thionyl chloride (0.76 mL, 10.4 mmol) in anhydrous methanol (30 mL), stirred at room temperature for 18 h. Mvi hydrochloride (1:1) was obtained as a as a white solid (642 mg, 84%). ¹H NMR (300 MHz, d₆-DMSO) δ 10.12 (br s, 2H), 7.17 (d, 1H), 6.90-6.81 (m, 2H), 4.54 (dd, 1H), 4.33-4.15 (m, 2H), 3.81 (s, 3H), 3.73 (s, 3H), 3.28 (dd, 1H), 3.20-3.07 (m, 1H).

### (3R)-6-Methoxy-1,2,3,4-tetrahydroisoquinoline-3-carboxamide (Mvii)

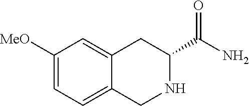 [SMILES: COc1ccc2c(c1)C[C@H](C(N)=O)NC2]

The compound was prepared by general procedure f with Mvi hydrochloride (1:1) (642 mg, 2.49 mmol) in 28-30% aqueous ammonium hydroxide (25 mL), stirred at room temperature for 18 h. The desired product Mvii was obtained as a white solid (470 mg, 91%). ¹H NMR (300 MHz, d₆-DMSO) δ 7.39 (br s, 1H), 7.13 (br s, 1H), 6.96 (d, 1H), 6.75-6.65 (m, 2H), 3.94-3.78 (m, 2H), 3.70 (s, 3H), 3.39 (dd overlapped with H₂O, 1H), 2.90 (dd, 1H), 2.72 (dd, 1H).

### (3R)-6-Methoxy-2-methyl-1,2,3,4-tetrahydroisoquinoline-3-carboxamide (Mviii)

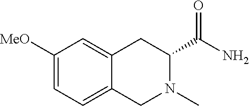 [SMILES: COc1ccc2c(c1)C[C@H](C(N)=O)N(C)C2]

The compound was prepared by general procedure g1 with Mvii (470 mg, 2.28 mmol), 37% aqueous formaldehyde (0.28 mL, 3.42 mmol), sodium triacetoxyborohydride (966 mg, 4.56 mmol) and acetic acid (0.13 mL, 2.28 mmol) in acetonitrile (22.5 mL) and tetrahydrofuran (22.5 mL), stirred at room temperature for 1 h. The desired product Mviii was obtained as a white solid (427 mg, 85%). ¹H NMR (300 MHz, d₆-DMSO) δ 7.37 (br s, 1H), 7.05 (br s, 1H), 6.96 (d, 1H), 6.74-6.65 (m, 2H), 3.81 (d, 1H), 3.70 (s, 3H), 3.40 (d, 1H), 3.04-2.86 (m, 2H), 2.81 (dd, 1H), 2.30 (s, 3H).

### ((3R)-6-Methoxy-2-methyl-1,2,3,4-tetrahydroisoquinolin-3-yl)methanamine (Mix)

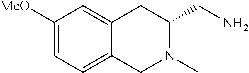

The compound was prepared by general procedure h with Mviii (220 mg, 1 mmol) and lithium aluminium hydride (114 mg, 3 mmol) in anhydrous tetrahydrofuran (20 mL), stirred at 50° C. for 4 h. After workup, the crude was purified by flash column chromatography (SiO₂, 10% methanol with 1% triethylamine in dichloromethane) to Mix as a yellow oil (138 mg, 67%). ¹H NMR (300 MHz, d₆-DMSO) δ 6.94 (d, 1H), 6.74-6.59 (m, 2H), 3.73-3.59 (m, 4H), 3.45 (d, 1H), 2.78-2.64 (m, 3H), 2.64-2.52 (m overlapped with -DMSOd₆, 2H), 2.28 (s, 3H).

### Methyl (2R)-3-(3-bromophenyl)-2-((tert-butoxycarbonyl)amino)propanoate (Nxi)

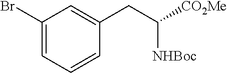 [SMILES: COC(=O)[C@@H](Cc1cccc(Br)c1)NC(=O)OC(C)(C)C]

To a solution of (2R)-3-(3-bromophenyl)-2-((tert-butoxycarbonyl)amino) propanoic acid Nx (5 g, 14.5 mmol) and potassium bicarbonate (2.91 g, 29 mmol) in N,N-dimethylformamide (36 mL) was added methyl iodide (1.36 mL, 21.8 mmol), and the reaction mixture was stirred at room temperature for 18 h. After reaction completion, the crude mixture was diluted with dichloromethane, extracted with brine, and the organic layer was concentrated under reduced pressure to give Nxi as a white solid (5.04 g, 97%). ¹H NMR (300 MHz, d₆-DMSO) δ 7.45 (s, 1H), 7.41 (br t, 1H), 7.32 (d, 1H), 7.29-7.20 (m, 2H), 4.25-4.13 (m, 1H), 3.62 (s, 3H), 3.02 (dd, 1H), 2.90-2.77 (m, 1H), 1.32 (s, 9H).

### Methyl (2R)-2-((tert-butoxycarbonyl)amino)-3-(3-ethylphenyl)propanoate (Nxii)

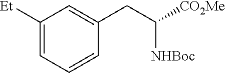 [SMILES: CCc1cccc(C[C@@H](NC(=O)OC(C)(C)C)C(=O)OC)c1]

A suspension of Nxi (1 g, 2.79 mmol), ethylboronic acid (248 mg, 3.35 mmol), palladium acetate (62.6 mg, 0.28 mmol), RuPhos (130 mg, 0.28 mmol) and potassium phosphate (1.2 g, 5.65 mmol) in anhydrous 1,4-dioxane (28 mL) was deoxygenated and then stirred at 100° C. for 18 h. After reaction completion, the crude was filtered through a short pad of celite, washed with tetrahydrofuran and the filtrate was concentrated under reduced pressure. The crude was purified by flash column chromatography (SiO₂, 10% ethyl acetate in n-hexane) to give Nxii as a colorless oil (828 mg, 97%). ¹H NMR (300 MHz, d₆-DMSO) δ 7.35-7.14 (m, 2H), 7.09-6.99 (m, 3H), 4.23-4.09 (m, 1H), 3.60 (s, 3H), 2.96 (dd, 1H), 2.82 (dd, 1H), 2.56 (q overlapped with d₆-DMSO, 2H), 1.32 (s, 9H), 1.16 (t, 3H).

### Methyl (2R)-2-amino-3-(3-ethylphenyl)propanoate hydrochloride (1:1) (Nii)

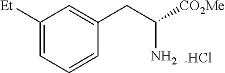 [SMILES: CCc1cccc(C[C@@H](N)C(=O)OC)c1 ; ".HCl"]

A suspension of Nxii (1.18 g, 3.84 mmol) in hydrochloric acid solution (4N in 1,4-dioxane) was stirred at room temperature for 0.5 h. After reaction completion, the crude mixture was concentrated under reduced pressure to give Nii hydrochloride (1:1) as a white solid (925 mg, 99%). ¹H NMR (300 MHz, d₆-DMSO) δ 8.43 (br s, 2H), 7.25 (t, 1H), 7.13 (d, 1H), 7.08-6.99 (m, 2H), 4.26 (t, 1H), 3.67 (s, 3H), 3.16-2.99 (m, 2H), 2.59 (q, 2H), 1.17 (t, 3H).

### Methyl (2R)-2-((ethoxycarbonyl)amino)-3-(3-ethylphenyl)propanoate (Niii)

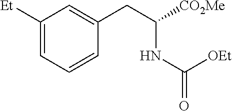 [SMILES: CCOC(=O)N[C@H](Cc1cccc(CC)c1)C(=O)OC]

The compound was prepared by general procedure b2 with Nii hydrochloride (1:1) (925 mg, 3.8 mmol), ethyl chloroformate (0.56 mL, 3.98 mmol) and pyridine (0.67 mL, 8.35 mmol) in dichloromethane (38 mL), stirred at 0° C. for 1 h. Niii was obtained as a yellow oil (1.1 g, quantitative yield). ¹H NMR (400 MHz, d₆-DMSO) δ 7.62 (d, 1H), 7.18 (t, 1H), 7.11-6.99 (m, 3H), 4.22-4.17 (m, 1H), 4.00-3.86 (m, 2H), 3.61 (s, 3H), 2.98 (dd, 1H), 2.82 (dd, 1H), 2.56 (q, 2H), 1.16 (t, 3H), 1.11 (t, 3H).

### 2-Ethyl 3-methyl (3R)-6-ethyl-3,4-dihydroisoquinoline-2,3(1H)-dicarboxylate (Niv)

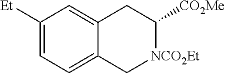

The compound was prepared by general procedure c with Niii (1.06 g, 3.8 mmol) and paraformaldehyde (1.25 g, 4.17 mmol) in a mixture of acetic acid (6 mL) and sulfuric acid (6 mL), stirred at room temperature for 18 h. After workup, the crude was purified by flash column chromatography (SiO₂, 20% ethyl acetate in n-hexane) to give Niv as a colorless oil (941 mg, 85%). ¹H NMR (400 MHz, d₆-DMSO) δ 7.17-7.00 (m, 3H), 4.99-4.80 (m, 1H), 4.71-4.31 (m, 2H), 4.20-3.99 (m, 2H), 3.59-3.55 (m, 1.8H), 3.54 (s, 1.2H), 3.23-3.01 (m, 2H), 2.55 (q, 2H), 1.24 (t, 2.4H), 1.15 (t, 3.6H).

### (3R)-6-Ethyl-1,2,3,4-tetrahydroisoquinoline-3-carboxylic acid hydrochloride (1:1) (Nv)

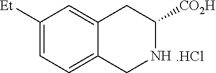 [SMILES: CCc1ccc2c(c1)C[C@H](C(=O)O)NC2 ; ".HCl"]

The compound was prepared by following general procedure d with Niv (941 mg, 3.23 mmol) in aqueous 6 N hydrogen chloride (32 mL), refluxed for 18 h. Nv hydrochloride (1:1) was obtained as a white solid (256 mg, 33%). ¹H NMR (300 MHz, d₆-DMSO) δ 9.73 (br s, 1H), 7.21-6.99 (m, 3H), 4.40-4.30 (m, 1H), 4.28 (s, 1H), 3.31-3.19 (m overlapped with H₂O, 2H), 3.07 (dd, 1H), 2.57 (q, 2H), 1.16 (t, 3H).

### Methyl (3R)-6-ethyl-1,2,3,4-tetrahydroisoquinoline-3-carboxylate hydrochloride (1:1) (Nvi)

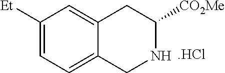 [SMILES: CCc1ccc2c(c1)C[C@H](C(=O)OC)NC2 ; ".HCl"]

The compound was prepared by general procedure e2 with Nv hydrochloride (1:1) (256 mg, 1.06 mmol), thionyl chloride (0.26 mL, 3.72 mmol) and triethylamine (0.16 mL, 1.17 mmol) in anhydrous methanol (10 mL), refluxed for 18 h. Nvi hydrochloride (1:1) was obtained as a yellow oil (237 mg, quantitative yield). ¹H NMR (300 MHz, d₆-DMSO) δ 6.99-6.87 (m, 3H), 3.96-3.76 (m, 2H), 3.65 (s, 3H), 3.59 (dd, 1H), 2.90 (dd, 1H), 2.77 (dd, 1H), 2.57-2.44 (m overlapped with d₆-DMSO, 2H), 1.13 (t, 3H).

### (3R)-6-Ethyl-1,2,3,4-tetrahydroisoquinoline-3-carboxamide (Nvii)

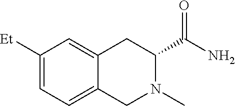 [SMILES: CCc1ccc2c(c1)C[C@H](C(N)=O)N(C)C2]

The compound was prepared by general procedure f with Nvi hydrochloride (1:1) (237 mg, 1.08 mmol) in 28-30% aqueous ammonium hydroxide (10 mL), stirred at room temperature for 18 h. Nvii was obtained as a white solid (152 mg, 70%). ¹H NMR (300 MHz, d₆-DMSO) δ 7.30 (br s, 1H), 7.04 (br s, 1H), 6.97-6.86 (m, 3H), 3.92-3.73 (m, 2H), 3.35-3.25 (m overlapped with H₂O, 1H), 2.83 (dd, 1H), 2.66 (dd, 1H), 2.57-2.49 (m overlapped with d₆-DMSO, 2H), 1.12 (t, 3H).

### (3R)-6-Ethyl-2-methyl-1,2,3,4-tetrahydroisoquinoline-3-carboxamide (Nviii)

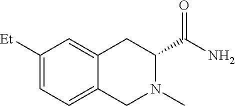 [SMILES: CCc1ccc2c(c1)C[C@H](C(N)=O)N(C)C2]

The compound was prepared by general procedure g1 with Nvii (152 mg, 0.74 mmol), 37% aqueous formaldehyde (0.09 mL, 1.12 mmol), sodium triacetoxyborohydride (315 mg, 1.49 mmol) and acetic acid (43 μL, 0.74 mmol) in acetonitrile (7.5 mL) and tetrahydrofuran (7.5 mL), stirred at room temperature for 2 h. Nviii was obtained as a white solid (130 mg, 80%). ¹H NMR (300 MHz, d₆-DMSO) δ 7.37 (br s, 1H), 7.05 (br s, 1H), 6.99-6.90 (m, 3H), 3.84 (d, 1H), 3.43 (d, 1H), 3.02 (dd, 1H), 2.97-2.73 (m, 2H), 2.58-2.51 (m overlapped with d₆-DMSO, 2H), 2.30 (s, 3H), 1.15 (t, 3H).

### ((3R)-6-Ethyl-2-methyl-1,2,3,4-tetrahydroisoquinolin-3-yl)methanamine (Nix)

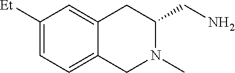

The compound was prepared by general procedure h with Nviii (130 mg, 0.6 mmol) and lithium aluminium hydride (68 mg, 1.79 mmol) in anhydrous tetrahydrofuran (12 mL), stirred at 60° C. for for 22 h. After workup, the crude was purified by flash column chromatography (SiO₂, 10% methanol with 1% triethylamine in dichloromethane) to give Nix as a beige solid (102 mg, 84%). ¹H NMR (300 MHz, d₆-DMSO) δ 6.99-6.92 (m, 3H), 3.76 (d, 1H), 3.66 (d, 1H), 3.10-2.90 (m, 1H), 2.93-2.67 (m, 4H), 2.67-2.52 (m overlapped with d₆-DMSO, 2H), 2.27 (s, 3H), 1.15 (t, 3H).

### Methyl 3-fluorophenylalaninate hydrochloride (1:1) (Oii)

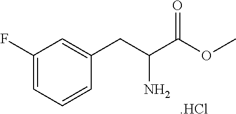

The compound was prepared by general procedure a with 3-fluoro-dl-phenylalanine Oi (0.5 g, 2.73 mmol) and thionyl chloride (0.218 mL, 3 mmol) in anhydrous methanol (4 mL), refluxed for 16 h. Oii hydrochloride (1:1) was obtained as a brown solid (0.538 g, quantitative yield). ¹H NMR (CD₃OD, 300 MHz) δ 7.44-7.35 (m, 1H), 7.12-7.03 (m, 3H), 4.37 (dd, 1H), 3.81 (s, 3H), 3.34-3.16 (m, 2H).

### Methyl N-(ethoxycarbonyl)-3-fluorophenylalaninate (Oiii)

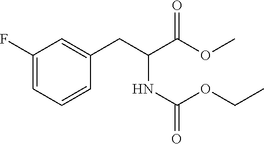 [SMILES: CCOC(=O)NC(Cc1cccc(F)c1)C(=O)OC]

The compound was prepared by general procedure b2 with Oii hydrochloride (1:1) (0.538 g, 2.73 mmol), ethyl chloroformate (0.286 mL, 3 mmol) and pyridine (0.484 mL, 6.01 mmol) in dichloromethane (8.9 mL), stirred at room temperature for 2 h. Oiii was obtained as a brown liquid (0.654 g, 89%). ¹H NMR (CDCl₃, 300 MHz) δ 7.29-7.20 (m, overlapped CDCl₃, 1H), 6.97-6.88 (m, 2H), 6.83 (d, 1H), 5.18 (br d, 1H), 4.63 (br q, 1H), 4.10 (q, 2H), 3.72 (s, 3H), 3.17-3.00 (m, 2H), 1.22 (t, 3H).

### 2-Ethyl 3-methyl 6-fluoro-3,4-dihydroisoquinoline-2,3(1H)-dicarboxylate (Oiv)

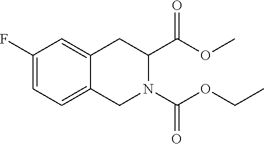 [SMILES: CCOC(=O)N1Cc2ccc(F)cc2CC1C(=O)OC]

The compound was prepared by general procedure c with Oiii (0.654 g, 2.43 mmol) and paraformaldehyde (76.5 mg, 2.55 mmol) in a mixture of acetic acid (2.35 mL) and sulfuric acid (0.783 mL), stirred at room temperature for 16 h. Oiv was obtained as a brown liquid (0.683 g, quantitative yield). ¹H NMR (CDCl₃, 300 MHz) δ 7.13-7.02 (m, 1H), 6.93-6.82 (m, 2H), 5.16 (dd, 0.5H), 4.93 (t, 0.5H), 4.72 (dd, 1H), 4.50 (dd, 1H), 4.26-4.14 (m, 2H), 3.62 (s, 3H), 3.27-3.09 (m, 2H), 1.32 (t, 1.5H), 1.24 (t, 1.5H).

### 6-Fluoro-1,2,3,4-tetrahydroisoquinoline-3-carboxylic acid hydrochloride (1:1) (Ov)

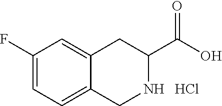 [SMILES: O=C(O)C1Cc2cc(F)ccc2CN1 ; Cl]

The compound was prepared by general procedure d with Oiv (2.27 g, 8.07 mmol) in aqueous 6 N hydrogen chloride (32.7 mL), refluxed for 48 h. Ov hydrochloride (1:1) was obtained as a brown solid (1.74 g, 93%). ¹H NMR (CD₃OD, 300 MHz) δ 7.29 (dd, 1H), 7.12-7.03 (m, 2H), 4.45-4.35 (m, 3H), 3.50 (dd, 1H), 3.24 (dd, 1H).

### Methyl 6-fluoro-1,2,3,4-tetrahydroisoquinoline-3-carboxylate (Ovi)

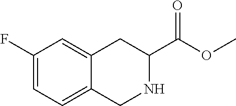

The compound was prepared by general procedure e1 with Ov hydrochloride (1:1) (1.74 g, 7.51 mmol) and sulfuric acid (0.802 mL, 15 mmol) in anhydrous methanol (39.8 mL), refluxed for 16 h. Ovi was obtained as a brown liquid (1.41 g, 90%). ¹H NMR (CDCl₃, 300 MHz) δ 6.97 (dd, 1H), 6.87-6.77 (m, 2H), 4.12-3.96 (m, 2H), 3.76 (s, 3H), 3.72 (dd, 1H), 3.06 (dd, 1H), 2.94 (dd, 1H).

### 6-Fluoro-1,2,3,4-tetrahydroisoquinoline-3-carboxamide (Ovii)

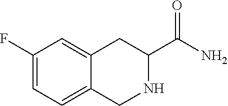 [SMILES: NC(=O)C1Cc2cc(F)ccc2CN1]

The compound was prepared by general procedure f with Ovi (1.36 g, 6.5 mmol) in 28-30% aqueous ammonium hydroxide (18.1 mL, 130 mmol), stirred at room temperature for 16 h. Ovii was obtained as a white solid (1.02 g, 81%). ¹H NMR (CD₃OD, 300 MHz) δ 7.11-7.03 (m, 1H), 6.92-6.83 (m, 2H), 4.03 (d, 1H), 3.94 (d, 1H), 3.60-3.52 (m, 1H), 3.09-2.98 (m, 1H), 2.94-2.82 (m, 1H).

### 6-Fluoro-2-methyl-1,2,3,4-tetrahydroisoquinoline-3-carboxamide (Oviii)

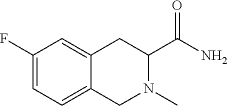

The compound was prepared by general procedure g1 with 6 Ovii (1.02 g, 5.26 mmol), 37% aqueous formaldehyde (1.38 mL, 18.4 mmol) and sodium triacetoxyborohydride (4.46 g, 21.1 mmol) in acetonitrile (76.7 mL), stirred at room temperature for 16 h. Oviii was obtained as a white solid (0.718 g, 66%). ¹H NMR (CD₃OD, 300 MHz) δ 7.13-7.03 (m, 1H), 6.93-6.82 (m, 2H), 4.00-3.88 (m, 1H), 3.58-3.47 (m, 1H), 3.20-2.92 (m, 3H), 2.48-2.40 (m, 3H).

### 1-(6-Fluoro-2-methyl-1,2,3,4-tetrahydroisominolin-3-yl)methanamine (Oix)

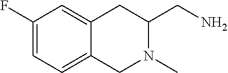

The compound was prepared by general procedure h with Oviii (0.3 g, 1.44 mmol) and lithium aluminium hydride (0.164 g, 4.32 mmol) in anhydrous tetrahydrofuran (28.7 mL), refluxed for 16 h. After workup, the crude was concentrated to give Oix as a yellow liquid (0.224 g, 80%). ¹H NMR (CDCl₃, 300 MHz) δ 6.97 (dd, 1H), 6.86-6.75 (m, 2H), 3.85-3.59 (m, 2H), 3.00-2.64 (m, 5H), 2.44-2.30 (br s, 3H).

### Methyl (2R)-2-amino-3-(3-fluorophenyl)propanoate hydrochloride (1:1) (Pii)

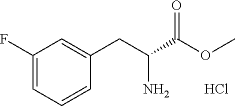

The compound was prepared by general procedure a with 3-fluoro-D-phenylalanine Pi (0.8 g, 4.37 mmol) and thionyl chloride (0.349 mL, 4.8 mmol) in anhydrous methanol (43.7 mL), refluxed for 16 h. Pii hydrochloride (1:1) was obtained as a white solid (0.976 g, 96%). ¹H NMR (CD₃OD, 400 MHz) δ 7.40 (ddd, 1H), 7.11-7.03 (m, 3H), 4.36 (dd, 1H), 3.82 (s, 3H), 3.32-3.25 (m, overlapped with CD₃OD, 1H), 3.18 (dd, 1H).

### Methyl (2R)-3-(3-fluorophenyl)-2-[(methoxycarbonyl)amino]propanoate (Piii)

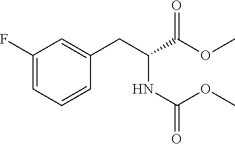 [SMILES: COC(=O)N[C@H](Cc1cccc(F)c1)C(=O)OC]

The compound was prepared by general procedure b1 with Pii hydrochloride (1:1) (0.976 g, 4.18 mmol), methyl chloroformate (0.339 mL, 4.39 mmol) and sodium bicarbonate (0.772 g, 9.19 mmol) in dichloromethane (41.8 mL), stirred at room temperature for 16 h. Piii was obtained as a yellow liquid (1.06 g, quantitative yield). ¹H NMR (CDCl₃, 400 MHz) δ 7.24 (ddd, overlapped with CDCl₃, 1H), 6.96-6.88 (m, 2H), 6.83 (dt, 1H), 5.25 (br d, 1H), 4.63 (q, 1H), 3.72 (s, 3H), 3.65 (s, 3H), 3.09 (ddd, 2H).

### Dimethyl (3R)-6-fluoro-3,4-dihydroisoquinoline-2,3(1H)-dicarboxylate (Piv)

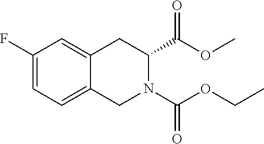 [SMILES: CCOC(=O)N1Cc2ccc(F)cc2C[C@@H]1C(=O)OC]

The compound was prepared by general procedure c with Piii (1.06 g, 4.17 mmol) and paraformaldehyde (0.188 g, 6.26 mmol) in a mixture of acetic acid (5.20 mL) and sulfuric acid (0.445 mL), stirred at room temperature for 16 h. Piv was obtained as a yellow liquid (5.2 g, 67%). ¹H NMR (CDCl₃, 400 MHz) δ 7.11-6.93 (m, 3H), 5.23-5.11 (m, 0.6H), 5.03-4.90 (m, 0.4H), 4.78-4.63 (m, 1H), 4.55-4.37 (m, 1H), 3.83-3.71 (m, 3H), 3.62 (s, 3H), 3.30-3.08 (m, 2H), 2.33-2.21 (m, 3H).

### (3R)-6-Fluoro-1,2,3,4-tetrahydroisoquinoline-3-carboxylic acid hydrochloride (1:1) (Pv)

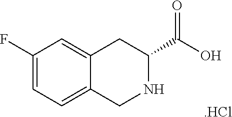 [SMILES: O=C(O)[C@H]1Cc2cc(F)ccc2CN1 ; ".HCl"]

The compound was prepared by general procedure d with Piv (0.892 g, 3.34 mmol) in aqueous 6 N hydrogen chloride (33.4 mL), refluxed for 48 h. Pv hydrochloride (1:1) was obtained as a white solid (0.773 g, quantitative yield). ¹H NMR (CD₃OD, 400 MHz) δ 7.30 (dd, 1H), 7.11-7.04 (m, 2H), 4.55-4.36 (m, 3H), 3.49 (dd, 1H), 3.24 (dd, 1H).

### Methyl (3R)-6-fluoro-1,2,3,4-tetrahydroisoquinoline-3-carboxylate (Pvi)

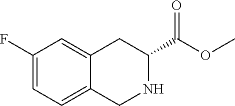

The compound was prepared by general procedure e2 with Pv hydrochloride (1:1) (0.804 g, 3.47 mmol), thionyl chloride (0.881 mL, 12.1 mmol) and triethylamine (0.532 mL, 3.82 mmol) in anhydrous methanol (34.7 mL), stirred at room temperature for 16 h. After workup, the crude was purified by flash column chromatography (SiO₂, dichloromethane/methanol=99/1) to give Pvi as a yellow liquid (0.661 g, 91%). ¹H NMR (CDCl₃, 400 MHz) δ 6.99 (dd, 1H), 6.86 (dd, 1H), 6.81 (dd, 1H), 4.10 (d, 1H), 4.03 (d, 1H), 3.78 (s, 3H), 3.73 (dd, 1H), 3.07 (dd, 1H), 2.96 (dd, 1H).

### (3R)-6-Fluoro-1,2,3,4-tetrahydroisoquinoline-3-carboxamide (Pvii)

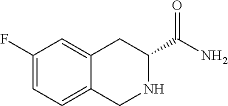 [SMILES: NC(=O)[C@H]1Cc2cc(F)ccc2CN1]

The compound was prepared by general procedure f with Pvi (0.661 g, 3.16 mmol) in 28-30% aqueous ammonium hydroxide (31.6 mL), stirred at room temperature for 16 h. Pvii was obtained as a white solid (0.462 g, 75%). ¹H NMR (CD₃OD, 400 MHz) δ 7.07 (dd, 1H), 6.91-6.84 (m, 2H), 4.03 (d, 1H), 3.95 (d, 1H), 3.55 (dd, 1H), 3.03 (dd, 1H), 2.88 (dd, 1H).

### (3R)-6-Fluoro-2-methyl-1,2,3,4-tetrahydroisoquinoline-3-carboxamide (Pviii)

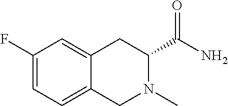

The compound was prepared by general procedure g1 with Pvii (0.34 g, 1.75 mmol), 37% aqueous formaldehyde (0.197 mL, 2.62 mmol), sodium triacetoxyborohydride (0.741 g, 3.5 mmol) and acetic acid (0.1 mL, 1.74 mmol) in acetonitrile (8.7 mL) and tetrahydrofuran (8.7 mL), stirred at room temperature for 16 h. Pviii was obtained as a white solid (0.355 g, 97%). ¹H NMR (CD₃OD, 400 MHz) δ 7.09 (dd, 1H), 6.92-6.85 (m, 2H), 3.97 (d, 1H), 3.57 (d, 1H), 3.19 (dd, 1H), 3.10 (dd, 1H), 3.01 (dd, 1H), 2.47 (s, 3H).

### [(3R)-6-Fluoro-2-methyl-1,2,3,4-tetrahydroisoquinolin-3-yl]methanamine (Pix)

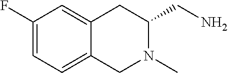

The compound was prepared by general procedure h with Pviii (0.355 g, 1.7 mmol) and lithium aluminium hydride (0.194 g, 5.11 mmol) in anhydrous tetrahydrofuran (17 mL), refluxed for 48 h. After workup, the crude was purified by flash column chromatography (SiO₂, dichloromethane/methanol/ammonium hydroxide solution=93/6.65/0.35) to give Pix as a yellow liquid (0.233 g, 70%). ¹H NMR (CDCl₃, 400 MHz) δ 6.97 (dd, 1H), 6.83 (dd, 1H), 6.79 (dd, 1H), 3.81 (d, 1H), 3.64 (d, 1H), 2.84 (dd, 2H), 2.77 (d, 2H), 2.70-2.63 (m, 1H), 2.39 (s, 3H).

### Methyl 2-amino-3-(3-chlorophenyl)propanoate hydrochloride (1:1) (Qii)

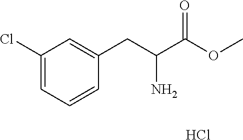

The compound was prepared by general procedure a with 3-chlorophenylalanine Qi (4 g, 20 mmol) and thionyl chloride (1.61 mL, 22 mmol) in anhydrous methanol (24.8 mL), refluxed for 16 h. Qii hydrochloride (1:1) was obtained as a brown solid (4.25 g, 85%). ¹H NMR (CD₃OD, 300 MHz) δ 7.40-7.32 (m, 3H), 7.21 (dt, 1H), 4.36 (t, 1H), 3.81 (s, 3H), 3.28 (dd, overlapped with CD₃OD, 1H), 3.18 (dd, 1H).

### Methyl 3-(3-chlorophenyl)-2-[(ethoxycarbonyl)amino]propanoate (Qiii)

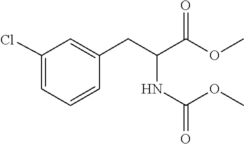 [SMILES: COC(=O)NC(Cc1cccc(Cl)c1)C(=O)OC]

The compound was prepared by general procedure b2 with Qii hydrochloride (1:1) (4.25 g, 17 mmol), ethyl chloroformate (1.78 mL, 18.7 mmol) and pyridine (3.01 mL, 37.4 mmol) in dichloromethane (64.5 mL), stirred at room temperature for 2 h. Qiii was obtained as a brown liquid (4.74 g, 98%). ¹H NMR (CDCl₃, 300 MHz) δ 7.23-7.20 (m, 2H), 7.11 (s, 1H), 7.02-6.98 (m, 1H), 5.19 (br d, 1H), 4.62 (br q, 1H), 4.10 (q, 2H), 3.72 (s, 3H), 3.15-2.97 (m, 2H), 1.22 (t, 3H).

### 2-Ethyl 3-methyl 6-chloro-3,4-dihydroisoquinoline-2,3(1H)-dicarboxylate (Qiv)

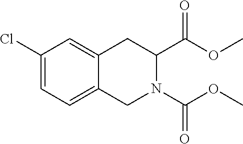 [SMILES: COC(=O)C1Cc2cc(Cl)ccc2CN1C(=O)OC]

The compound was prepared by general procedure c with Qiii (4.7 g, 16.4 mmol) and paraformaldehyde (0.518 g, 17.3 mmol) in a mixture of acetic acid (15.9 mL) and sulfuric acid (5.3 mL), stirred at room temperature for 16 h. Qiv was obtained as a colorless liquid (3.55 g, 73%). ¹H NMR (CDCl₃, 300 MHz) δ 7.20-7.00 (m, 3H), 5.18 (dd, 0.7H), 4.95 (dd, 0.3H), 4.74 (dd, 1H), 4.50 (dd, 1H), 4.28-4.15 (m, 2H), 3.63 (s, 3H), 3.28-3.07 (m, 2H), 1.36-1.21 (m, 3H).

### 6-Chloro-1,2,3,4-tetrahydroisoquinoline-3-carboxylic acid hydrochloride (1:1) (Qv)

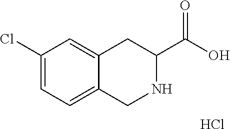 [SMILES: O=C(O)C1Cc2cc(Cl)ccc2CN1 ; Cl]

The compound was prepared by general procedure d with Qiv (3.55 g, 11.9 mmol) in aqueous 8 N hydrogen chloride (119 mL), refluxed for 48 h. Qv hydrochloride (1:1) was obtained as a white solid (2.26 g, 76%). ¹H NMR (CD₃OD, 300 MHz) δ 7.36 (s, 1H), 7.32 (dd, 1H), 7.26 (d, 1H), 4.50-4.37 (m, 3H), 3.49 (dd, 1H), 3.23 (dd, 1H).

### Methyl 6-chloro-1,2,3,4-tetrahydroisoquinoline-3-carboxylate (Qvi)

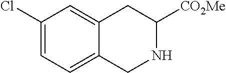 [SMILES: COC(=O)C1Cc2cc(Cl)ccc2CN1]

The compound was prepared by general procedure e2 with Qv hydrochloride (1:1) (1.97 g, 7.94 mmol), thionyl chloride (2.02 mL, 27.8 mmol) and triethylamine (1.22 mL, 8.73 mmol) in anhydrous methanol (79 mL), stirred at room temperature for 18 h. After workup, the crude was purified by flash column chromatography (SiO₂, 3% methanol in dichloromethane) to give Qvi as a colorless oil (1.42 mg, 73%). ¹H NMR (300 MHz, d₆-DMSO) δ 7.20 (s, 1H), 7.16 (dd, 1H), 7.06 (d, 1H), 3.98-3.78 (m, 2H), 3.70-3.63 (m, 1H), 3.66 (s, 3H), 2.96 (dd, 1H), 2.88-2.73 (m, 2H).

### 6-Chloro-1,2,3,4-tetrahydroisoquinoline-3-carboxamide (Qvii)

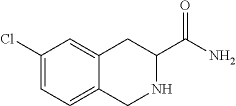 [SMILES: NC(=O)C1Cc2cc(Cl)ccc2CN1]

The compound was prepared by general procedure f with Qvi (775 mg, 2.96 mmol) in 28-30% aqueous ammonium hydroxide (30 mL), stirred at room temperature for 18 h. Qvii was obtained as a white solid (908 mg, 80%). ¹H NMR (300 MHz, d₆-DMSO) δ 7.33 (br s, 1H), 7.18 (s, 1H), 7.18-7.01 (m, 3H), 3.95-3.76 (m, 2H), 3.40-3.29 (m overlapped with H₂O, 1H), 2.89 (dd, 1H), 2.71 (dd, 1H), 2.60 (br s, 1H).

### 6-Chloro-2-methyl-1,2,3,4-tetrahydroisoquinoline-3-carboxamide (Qviii)

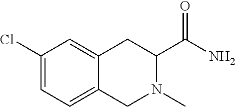

The compound was prepared by general procedure g1 with Qvii (908 mg, 4.31 mmol), 37% aqueous formaldehyde (1.22 mL, 15.1 mmol), sodium triacetoxyborohydride (3.65 g, 17.3 mmol) and acetic acid (0.517 mL, 8.62 mmol) in acetonitrile (21.5 mL) and tetrahydrofuran (21.5 mL), stirred at room temperature for 2 h. After workup, the crude was purified by flash column chromatography (SiO₂, 3-5% methanol in dichloromethane) to give Qviii as a white solid (936 mg, 97%). ¹H NMR (300 MHz, d₆-DMSO) δ 7.40 (br s, 1H), 7.20 (s, 1H), 7.16 (dd, 1H), 7.14-7.04 (m, 2H), 3.86 (d, 1H), 3.47 (d, 1H), 3.08 (dd, 1H), 2.99-2.80 (m, 2H), 2.31 (s, 3H).

### (6-Chloro-2-methyl-1,2,3,4-tetrahydroisoquinolin-3-yl)methanamine (Qix)

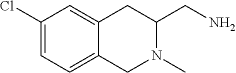

The compound was prepared by general procedure h with Qviii (0.355 g, 1.7 mmol) and lithium aluminium hydride (50.7 mg, 1.34 mmol) in anhydrous tetrahydrofuran (8.8 mL), refluxed for 16 h. After workup, the crude was concentrated to give Qix as a yellow liquid (72 mg, 77%). ¹H NMR (CDCl₃, 300 MHz) δ 7.09-7.03 (m, 2H), 6.92 (d, 1H), 3.77 (d, 1H), 3.61 (d, 1H), 2.80 (dd, 2H), 2.75-2.60 (m, 3H), 2.35 (s, 3H).

### Methyl (2R)-2-amino-3-(3-chlorophenyl)propanoate hydrochloride (1:1) (Rii)

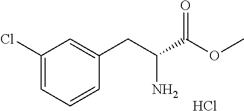

The compound was prepared by general procedure a with (2R)-2-amino-3-(3-chlorophenyl) propanoic acid Ri (5 g, 25 mmol) and thionyl chloride (2 mL, 27.6 mmol) in anhydrous methanol (62.6 mL), refluxed for 16 h. Rii hydrochloride (1:1) was obtained as a white solid (6.26 g, quantitative yield). ¹H NMR (CD₃OD, 400 MHz) δ 7.40-7.32 (m, 3H), 7.21 (dt, 1H), 4.36 (t, 1H), 3.82 (s, 3H), 3.27 (dd, overlapped with CD₃OD, 1H), 3.17 (dd, 1H).

### Methyl (2R)-3-(3-chlorophenyl)-2-[(methoxycarbonyl)amino]propanoate (Riii)

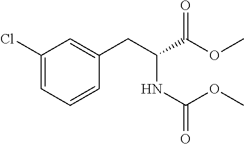 [SMILES: COC(=O)N[C@H](Cc1cccc(Cl)c1)C(=O)OC]

The compound was prepared by general procedure b1 with Rii hydrochloride (1:1) (6.42 g, 25.7 mmol), methyl chloroformate (2.08 mL, 27 mmol) and sodium bicarbonate (4.74 g, 56.5 mmol) in dichloromethane (64.2 mL), stirred at room temperature for 16 h. Riii was obtained as a colorless liquid (6.76 g, 97%). ¹H NMR (CDCl₃, 400 MHz) δ 7.23-7.20 (m, 2H), 7.11 (s, 1H), 7.02-6.98 (m, 1H), 5.23 (br d, 1H), 4.63 (br q, 1H), 3.72 (s, 3H), 3.66 (s, 3H), 3.07 (ddd, 2H).

### Dimethyl (3R)-6-chloro-3,4-dihydroisoquinoline-2,3(1H)-dicarboxylate (Riv)

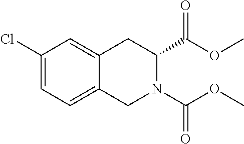 [SMILES: COC(=O)[C@H]1Cc2cc(Cl)ccc2CN1C(=O)OC]

The compound was prepared by general procedure c with Riii (1.06 g, 4.17 mmol) and paraformaldehyde (1.12 g, 37.3 mmol) in a mixture of acetic acid (31.1 mL) and sulfuric acid (3.98 mL), stirred at room temperature for 16 h. Riv was obtained as a yellow liquid (6.45 g, 88%). ¹H NMR (CDCl₃, 400 MHz) δ 7.25-7.00 (m, 3H), 5.17 (dd, 0.5H), 4.97 (dd, 0.5H), 4.73 (dd, 1H), 4.51 (dd, 1H), 3.85-3.72 (m, 3H), 3.63 (s, 3H), 3.34-3.09 (m, 2H).

### (3R)-6-Chloro-1,2,3,4-tetrahydroisoquinoline-3-carboxylic acid hydrochloride (1:1) (Rv)

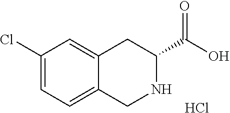 [SMILES: O=C(O)[C@H]1Cc2cc(Cl)ccc2CN1 ; Cl]

The compound was prepared by general procedure d with Riv (4.85 g, 17.1 mmol) in aqueous 12 N hydrogen chloride (33.4 mL), refluxed for 16 h. Rv hydrochloride (1:1) was obtained as a white solid (3.05 g, 72%). ¹H NMR (CD₃OD, 400 MHz) δ 7.37 (s, 1H), 7.32 (dd, 1H), 7.26 (d, 1H), 4.50-4.36 (m, 3H), 3.49 (dd, 1H), 3.23 (dd, 1H).

### Methyl (3R)-6-chloro-1,2,3,4-tetrahydroisoquinoline-3-carboxylate (Rvi)

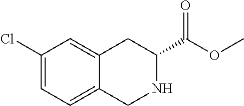

The compound was prepared by general procedure e2 with Rv (1:1) (3.05 g, 12.3 mmol), thionyl chloride (3.12 mL, 43 mmol) and triethylamine (1.89 mL, 13.5 mmol) in anhydrous methanol (61.5 mL), stirred at room temperature for 16 h. After workup, the crude was purified by flash column chromatography (SiO₂, dichloromethane/methanol=98/2) to give Rvi as a colorless liquid (2.42 g, 87%). ¹H NMR (CDCl₃, 400 MHz) δ 7.13-7.09 (m, 2H), 6.96 (d, 1H), 4.10 (d, 1H), 4.02 (d, 1H), 3.78 (s, 3H), 3.72 (dd, 1H), 3.06 (dd, 1H), 2.94 (dd, 1H).

### (3R)-6-Chloro-1,2,3,4-tetrahydroisoquinoline-3-carboxamide (Rvii)

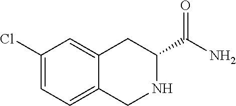 [SMILES: NC(=O)[C@H]1Cc2cc(Cl)ccc2CN1]

The compound was prepared by general procedure f with Rvi (1 g, 4.43 mmol) in 28-30% aqueous ammonium hydroxide (22.2 mL), stirred at room temperature for 16 h. Rvii was obtained as a white solid (0.794 g, 85%). ¹H NMR (CD₃OD, 400 MHz) δ 7.17-7.12 (m, 2H), 7.05 (d, 1H), 4.03 (d, 1H), 3.95 (d, 1H), 3.55 (dd, 1H), 3.02 (dd, 1H), 2.87 (dd, 1H).

### (3R)-6-Chloro-2-methyl-1,2,3,4-tetrahydroisoquinoline-3-carboxamide (Rviii)

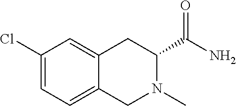

The compound was prepared by general procedure g1 with Rvii (0.794 g, 3.77 mmol), 37% aqueous formaldehyde (0.424 mL, 5.65 mmol), sodium triacetoxyborohydride (1.6 g, 7.53 mmol) and acetic acid (0.216 mL, 3.77 mmol) in acetonitrile (18.8 mL) and tetrahydrofuran (18.8 mL), stirred at room temperature for 16 h. Rviii was obtained as a white solid (0.825 g, 97%). ¹H NMR (CD₃OD, 400 MHz) δ 7.17-7.12 (m, 2H), 7.07 (d, 1H), 3.95 (d, 1H), 3.54 (d, 1H), 3.16 (dd, 1H), 3.12-2.94 (m, 2H), 2.44 (s, 3H).

### ((3R)-6-Chloro-2-methyl-1,2,3,4-tetrahydroisoquinolin-3-yl)methanamine (Rix)

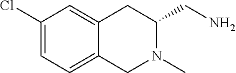

The compound was prepared by general procedure h with Rviii (0.355 g, 1.7 mmol) and lithium aluminium hydride (0.101 g, 2.67 mmol) in anhydrous tetrahydrofuran (8.9 mL), refluxed for 24 h. After workup, the crude was purified by flash column chromatography (SiO₂, dichloromethane/methanol/ammonium hydroxide solution=92/7.6/0.4) to give the desired product Rix as a yellow liquid (0.127 g, 64%). ¹H NMR (CDCl₃, 400 MHz) δ 7.03-6.99 (m, 2H), 6.87 (d, 1H), 3.72 (d, 1H), 3.55 (d, 1H), 2.80-2.69 (m, 2H), 2.67 (d, 2H), 2.60-2.52 (m, 1H), 2.31 (s, 3H).

### Methyl (2R)-2-amino-3-(3-bromophenyl)propanoate hydrochloride (1:1) (Sii)

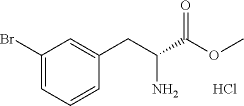

The compound was prepared by general procedure a with 3-bromo-D-phenylalanine Si (5 g, 20.5 mmol) and thionyl chloride (1.63 mL, 22.5 mmol) in anhydrous methanol (68.3 mL), refluxed for 16 h. Sii hydrochloride (1:1) was obtained as a as a white solid (6.03 g, quantitative yield). ¹H NMR (CD₃OD, 400 MHz) δ 7.52-7.47 (m, 2H), 7.30 (td, 1H), 7.25 (dt, 1H), 4.34 (dd, 1H), 3.81 (s, 3H), 3.26 (dd, 1H), 3.17 (dd, 1H).

### Methyl (2R)-3-(3-bromophenyl)-2-[(methoxycarbonyl)amino]propanoate (Siii)

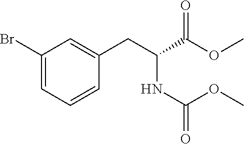 [SMILES: COC(=O)N[C@H](Cc1cccc(Br)c1)C(=O)OC]

The compound was prepared by general procedure b1 with Sii hydrochloride (1:1) (6.6 g, 22.4 mmol), methyl chloroformate (1.82 mL, 23.5 mmol) and sodium bicarbonate (4.14 g, 49.3 mmol) in dichloromethane (56 mL), stirred at room temperature for 16 h. Siii was obtained as a colorless liquid (6.8 g, 96%). ¹H NMR (CDCl₃, 400 MHz) δ 7.38 (ddd, 1H), 7.28 (t, 1H), 7.16 (t, 1H), 7.06 (dt, 1H), 5.17 (br d, 1H), 4.63 (q, 1H), 3.73 (s, 3H), 3.67 (s, 3H), 3.07 (ddd, 2H).

### Dimethyl (3R)-6-bromo-3,4-dihydroisoquinoline-2,3(1H)-dicarboxylate (Siv)

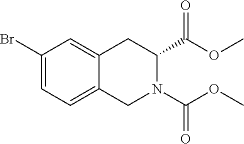 [SMILES: COC(=O)[C@H]1Cc2cc(Br)ccc2CN1C(=O)OC]

The compound was prepared by general procedure c with Siii (6.8 g, 21.5 mmol) and paraformaldehyde (0.969 g, 32.3 mmol) in a mixture of acetic acid (26.9 mL) and sulfuric acid (3.44 mL), stirred at room temperature for 16 h. After workup, the crude was concentrated to give Siv as a colorless liquid (5.56 g, 79%). ¹H NMR (CDCl₃, 400 MHz) δ 7.45-7.28 (m, 1.7H), 7.12-6.94 (m, 1.3H), 5.25 (dd, 0.2H), 5.17 (dd, 0.4H), 5.07-5.03 (m, 0.1H), 4.97 (dd, 0.3H), 4.80-4.66 (m, 1H), 4.54-4.40 (m, 1H), 3.85-3.73 (m, 3H), 3.64 (s, 1.5H), 3.63 (s, 1.5H), 3.32-3.10 (m, 2H).

### (3R)-6-Bromo-1,2,3,4-tetrahydroisoquinoline-3-carboxylic acid hydrochloride (1:1) (Sv)

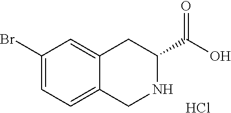 [SMILES: O=C(O)[C@H]1Cc2cc(Br)ccc2CN1 ; Cl]

The compound was prepared by general procedure d with Siv (5.56 g, 16.9 mmol) in aqueous 12 N hydrogen chloride (42.3 mL), refluxed for 48 h. Sv hydrochloride (1:1) was obtained as a white solid (3.04 g, 61%). ¹H NMR (CD₃OD, 400 MHz) δ 7.52 (s, 1H), 7.46 (dd, 1H), 7.19 (d, 1H), 4.56-4.29 (m, 3H), 3.53-3.42 (m, 1H), 3.28-3.19 (m, 1H).

### Methyl (3R)-6-bromo-1,2,3,4-tetrahydroisoquinoline-3-carboxylate (Svi)

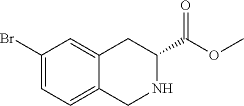

The compound was prepared by general procedure e2 with Sv hydrochloride (1:1) (3.04 g, 10.4 mmol), thionyl chloride (2.64 mL, 36.4 mmol) and triethylamine (1.60 mL, 11.4 mmol) in anhydrous methanol (34.7 mL), refluxed for 16 h. After workup, the crude was purified by flash column chromatography (SiO₂, dichloromethane/methanol=98/2) to give Svi as a yellow liquid (1.69 g, 60%). ¹H NMR (CDCl₃, 400 MHz) δ 7.27-7.23 (m, overlapped with CDCl₃, 2H), 6.89 (d, 1H), 4.07 (d, 1H), 3.99 (d, 1H), 3.77 (s, 3H), 3.71 (dd, 1H), 3.04 (dd, 1H), 2.93 (dd, 1H).

### Methyl (3R)-6-bromo-2-methyl-1,2,3,4-tetrahydroisoquinoline-3-carboxylate (Sxv)

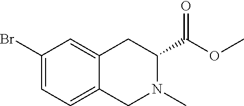 [SMILES: COC(=O)[C@H]1Cc2cc(Br)ccc2CN1C]

The compound was prepared by general procedure g1 with Svi (1.69 g, 6.24 mmol), 37% aqueous formaldehyde (0.703 mL, 9.37 mmol), sodium triacetoxyborohydride (2.65 g, 12.5 mmol) and acetic acid (0.358 mL, 6.25 mmol) in acetonitrile (31.2 mL) and tetrahydrofuran (31.2 mL), stirred at room temperature for 16 h. Sxv was obtained as a yellow liquid (1.78 g, 100%). ¹H NMR (CD₃OD, 400 MHz) δ 7.27-7.23 (m, overlapped with CDCl₃, 2H), 6.90 (d, 1H), 3.95 (d, 1H), 3.71 (s, 3H), 3.68 (d, overlapped with CH₃ at 3.71, 1H), 3.54 (t, 1H), 3.11-3.07 (m, 2H), 2.54 (s, 3H).

### [(3R)-6-Bromo-2-methyl-1,2,3,4-tetrahydroisoquinolin-3-yl]methanol (Sxvi)

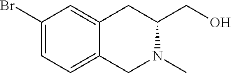

To a solution of Sxv (0.5 g, 1.76 mmol) in tetrahydrofuran (17.6 mL) was added 1 M lithium triethylborohydride in tetrahydrofuran (21.1 mL, 21.1 mmol). The solution was stirred at room temperature for 16 h, cooled to room temperature and added saturated NH₄Cl_{(aq)} (3 mL). The mixture was filtered with a pad of Celite and concentrated under reduced pressure. The residue was added 60 mL of brine and washed with dichloromethane (2× 60 mL). The organic layer was dried over MgSO₄₍ₛ₎, and the solvent was evaporated under reduced pressure. The crude was purified by flash column chromatography (SiO₂, dichloromethane/methanol=95/5) to give Sxvi as a yellow solid (0.266 g, 59%). ¹H NMR (CDCl₃, 400 MHz) δ 7.30-7.25 (m, overlapped with CDCl₃, 2H), 6.94 (d, 1H), 3.90 (d, 1H), 3.76 (dd, 1H), 3.70 (d, 1H), 3.60 (dd, 1H), 2.98-2.90 (m, 1H), 2.83-2.70 (m, 2H), 2.46 (s, 3H).

### [(3R)-6-Bromo-2-methyl-1,2,3,4-tetrahydroisoquinolin-3-yl]methyl methanesulfonate (Sxvii)

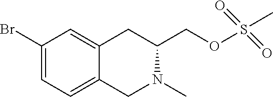 [SMILES: CN1Cc2ccc(Br)cc2C[C@@H]1COS(C)(=O)=O]

To a solution of Sxvi (84.5 mg, 0.33 mmol) in dichloromethane (6.6 mL) was added methanesulfonyl chloride (33.2 μL, 0.429 mmol) and trimethylamine (59.8 μL, 0.429 mmol). The solution was stirred at room temperature for 2 h. To the solution was added 30 mL of saturated NaHCO_{3(aq)} and extracted with dichloromethane (2× 60 mL). The organic layer was dried over MgSO₄₍ₛ₎, and the solvent was evaporated under reduced pressure. The crude was purified by a quick flash column chromatography (SiO₂, ethyl acetate/hexane=1/1) to give Sxvii as a yellow liquid (40.3 mg, 37%). ¹H NMR (CDCl₃, 400 MHz) δ 7.27-7.23 (m, overlapped with CDCl₃, 2H), 6.90 (d, 1H), 3.78 (d, 1H), 3.67 (dd, 1H), 3.61 (d, 1H), 3.55 (dd, 1H), 3.13 (s, 3H), 3.00 (dd, 1H), 2.95-2.84 (m, 2H), 2.49 (s, 3H).

### 3-(((3R)-6-Bromo-2-methyl-1,2,3,4-tetrahydroisoquinolin-3-yl)methyl)triaza-1,2-dien-2-ium-1-ide (Sxviii)

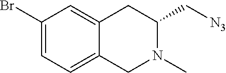 [SMILES: CN1Cc2ccc(Br)cc2C[C@@H]1CN=[N+]=[N-]]

To a solution of Sxvii (376 mg, 1.12 mmol) in N,N-dimethylformamide (22 mL) was added sodium azide (110 mg, 1.68 mmol), and the reaction mixture was stirred at room temperature for 18 h. After reaction completion, the crude was diluted with dichloromethane, extracted with brine, then the organic layer was concentrated under reduced pressure to give Sxviii as a brown oil (316 mg, quantitative yield). ¹H NMR (600 MHz, d₆-DMSO) δ 7.34 (d, 1H), 7.31 (dd, 1H), 7.04 (d, 1H), 3.73 (d, 1H), 3.55 (d, 1H), 3.50 (dd, 1H), 3.40 (dd, 1H), 2.84-2.78 (m, 2H), 2.67 (dd, 1H), 2.33 (s, 3H).

### ((3R)-6-Bromo-2-methyl-1,2,3,4-tetrahydroisoquinolin-3-yl)methanamine (Six)

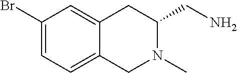

To a solution of Sxviii (100 mg, 0.356 mmol) in methanol (3.6 mL) was added sodium iodide (320 g, 2.14 mmol) at room temperature. After 10 minutes, trimethylsilyl chloride (0.273 mL, 2.14 mmol) was added to the reaction mixture, and stirred at room temperature for 3 h. After reaction completion, the reaction mixture was portion-wise poured into the pre-cooled saturated sodium thiosulfate pentahydrate (281 mg, 1.78 mmol) solution, then diluted with dichloromethane, extracted, and the aqueous layer was concentrated under reduced pressure. The crude was purified by flash column chromatography (SiO₂, 10-20% methanol with 1-2% triethylamine in dichloromethane) to give Six as a yellow syrup (47 mg, 52%). ¹H NMR (400 MHz, CD₃OD) δ 7.32 (s, 1H), 7.29 (dd, 1H), 7.01 (d, 1H), 3.84 (d, 1H), 3.72 (d, 1H), 2.96-2.82 (m, 4H), 2.76-2.67 (m, 1H), 2.40 (s, 3H).

### 7-Methyl-1,2,3,4-tetrahydroisoquinoline-3-carboxylic acid (Tv)

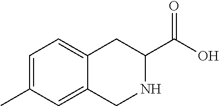

The compound was prepared by general procedure j2 with 2-amino-3-(4-methylphenyl) propanoic acid Ti (0.5 g, 2.79 mmol) and formaldehyde (1.68 mL, 22.3 mmol) in 48% aqueous hydrochloric acid (5 mL), stirred at 75° C. for 5 h. Tv hydrochloride (1:1) was obtained as a white solid (0.53 g, 99%). ¹H NMR (400 MHz, d₆-DMSO) δ 9.51 (br s, 2H), 7.27-6.93 (m, 3H), 4.38 (dd, 1H), 4.32-4.24 (m, 2H), 3.29-3.19 (m, 1H), 3.11-2.96 (m, 1H), 2.27 (s, 3H).

### Methyl 7-methyl-1,2,3,4-tetrahydroisoquinoline-3-carboxylate (Tvi)

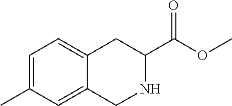

The compound was prepared by general procedure e2 with Tv hydrochloride (1:1) (0.536 g, 2.8 mmol) and thionyl chloride (1.73 mL, 23.8 mmol) in anhydrous methanol (30 mL), stirred at room temperature for overnight. Tvi was obtained as a white solid (0.542 g, 94%). ¹H NMR (400 MHz, d₆-DMSO) δ 9.84 (s, 1H), 7.21-7.00 (m, 3H), 4.56 (dd, 1H), 4.34-4.23 (m, 2H), 3.82 (s, 3H), 3.26 (dd, 1H), 3.08 (dd, 1H), 2.27 (s, 3H).

### 7-Methyl-1,2,3,4-tetrahydroisoquinoline-3-carboxamide (Tvii)

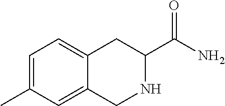

The compound was prepared by general procedure f with Tvi (0.542 g, 2.64 mmol) in 28-30% aqueous ammonium hydroxide (18.3 mL), stirred at room temperature for 17 h. Tvii was obtained as a white solid (0.287 g, 57%). ¹H NMR (400 MHz, d₆-DMSO) δ 7.31 (br s, 1H), 7.05 (br s, 1H), 7.00-6.87 (m, 2H), 6.83 (s, 1H), 3.96-3.75 (m, 2H), 3.39-3.25 (m overlapped with H₂O, 1H) 2.83 (d, 1H), 2.74-2.60 (m, 1H), 2.22 (s, 3H).

### 2,7-Dimethyl-1,2,3,4-tetrahydroisoquinoline-3-carboxamide (Tviii)

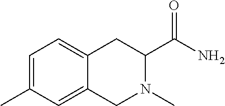

The compound was prepared by general procedure g1 with Tvii (0.287 g, 1.51 mmol), 37% aqueous formaldehyde (0.39 mL, 5.28 mmol) and sodium triacetoxyborohydride (1.28 g, 6.03 mmol) in acetonitrile (28 mL) and tetrahydrofuran (57 mL), stirred at room temperature for 17 h. After workup, the crude was purified by Isco Combi-Flash Companion column chromatography (0-5% methanol in CH₂Cl₂) to give Tviii as a white solid (0.242 g, 78.5%). ¹H NMR (400 MHz, CDCl₃) δ 7.10-6.97 (m, 2H), 6.92-6.87 (m, 2H), 5.32 (br s, 1H), 3.82 (d, 1H), 3.67 (d, 1H), 3.33-3.21 (m, 1H), 3.06-2.99 (m, 2H), 2.43 (s, 3H), 2.31 (s, 3H).

### (2,7-Dimethyl-1,2,3,4-tetrahydroisoquinolin-3-yl)methanamine (Tix)

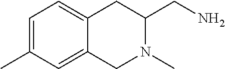

The compound was prepared by general procedure h with Tviii (0.242 g, 1.18 mmol) and lithium aluminium hydride (130 mg, 3.55 mmol) in anhydrous tetrahydrofuran (20 mL), stirred at 50° C. for 17 h. After workup, the crude was concentrated to give Tix (0.162 g, 72%). ¹H NMR (300 MHz, CDCl₃) δ 7.07-6.90 (m, 2H), 6.85 (s, 1H), 3.81 (d, 1H), 3.68 (d, 1H), 2.90-2.79 (m, 2H), 2.78-2.64 (m, 3H), 2.39 (s, 3H), 2.29 (s, 3H).

### Methyl 4-fluorophenylalaninate (Uii)

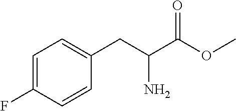

The compound was prepared by general procedure a with 4-fluoro-dl-phenylalanine Ui (0.1 g, 0.546 mmol) and thionyl chloride (43.6 μL, 0.601 mmol) in anhydrous methanol (0.8 mL), refluxed for 16 h. Uii was obtained as a as a white solid (0.108 g, quantitative yield). ¹H NMR (CD₃OD, 300 MHz) δ 7.28 (dd, 2H), 7.11 (dd, 2H), 4.32 (dd, 1H), 3.82 (s, 3H), 3.30-3.12 (m, 2H).

### Methyl N-(ethoxycarbonyl)-4-fluorophenylalaninate (Uiii)

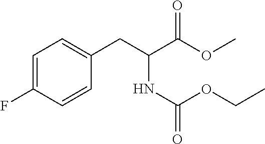 [SMILES: CCOC(=O)NC(Cc1ccc(F)cc1)C(=O)OC]

The compound was prepared by general procedure b2 with Uii (0.108 g, 0.546 mmol), ethyl chloroformate (57.2 μL, 0.601 mmol) and pyridine (96.8 μL, 1.20 mmol) in dichloromethane (1.79 mL), stirred at rt for 2 h. Uiii was obtained as a yellow liquid (6.88 g, quantitative yield). ¹H NMR (CDCl₃, 300 MHz) δ 7.06 (dd, 1H), 6.88-6.75 (m, 2H), 5.15 (dd, 0.5H), 4.93 (dd, 0.5H), 4.72 (d, 1H), 4.50 (dd, 1H), 4.25-4.10 (m, 2H), 3.59 (s, 3H), 3.25-3.00 (m, 2H), 1.30 (t, 1.5H), 1.23 (t, 1.5H).

### 2-Ethyl 3-methyl 7-fluoro-3,4-dihydroisoquinoline-2,3(1H)-dicarboxylate (Uiv)

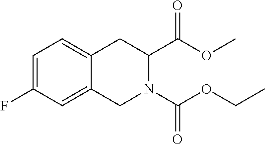 [SMILES: CCOC(=O)N1Cc2cc(F)ccc2CC1C(=O)OC]

The compound was prepared by general procedure c with Uiii (6.59 g, 24.5 mmol) and paraformaldehyde (0.772 g, 25.7 mmol) in a mixture of acetic acid (23.7 mL) and sulfuric acid (7.90 mL), stirred at room temperature for 16 h. After workup, the crude was concentrated to give Uiv as a yellow liquid (6.88 g, quantitative yield). ¹H NMR (CDCl₃, 300 MHz) δ 7.06 (dd, 1H), 6.88-6.75 (m, 2H), 5.15 (dd, 0.5H), 4.93 (dd, 0.5H), 4.72 (d, 1H), 4.50 (dd, 1H), 4.25-4.10 (m, 2H), 3.59 (s, 3H), 3.25-3.00 (m, 2H), 1.30 (t, 1.5H), 1.23 (t, 1.5H).

### 7-Fluoro-1,2,3,4-tetrahydroisoquinoline-3-carboxylic acid hydrochloride (1:1) (Uv)

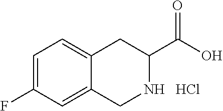 [SMILES: O=C(O)C1Cc2ccc(F)cc2CN1 ; Cl]

The compound was prepared by general procedure d with Uiv (0.726 g, 2.58 mmol) in aqueous 6 N hydrogen chloride (10.5 mL), refluxed for 48 h. Uv hydrochloride (1:1) was obtained as a brown solid (0.598 g, 98%). ¹H NMR (CD₃OD, 300 MHz) δ 7.33 (dd, 1H), 7.13-7.02 (m, 2H), 4.52-4.38 (m, 3H), 3.47 (dd, 1H), 3.19 (dd, 1H).

### Methyl 7-fluoro-1,2,3,4-tetrahydroisoquinoline-3-carboxylate (Uvi)

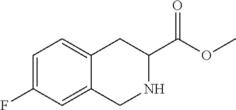

The compound was prepared by general procedure e1 Uv hydrochloride (1:1) (0.1 g, 0.432 mmol) and sulfuric acid (50 μL, 0.938 mmol) in methanol (2.5 mL), refluxed for 16 h. Uvi was obtained as a brown liquid (79.8 mg, 88%). ¹H NMR (CDCl₃, 300 MHz) δ 7.06 (dd, 1H), 6.85 (td, 1H), 6.73 (d, 1H), 4.16-4.00 (m, 2H), 3.77 (s, 3H), 3.72 (dd, 1H), 3.05 (dd, 1H), 2.90 (dd, 1H).

### 7-Fluoro-1,2,3,4-tetrahydroisoquinoline-3-carboxamide (Uvii)

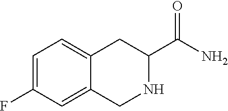 [SMILES: NC(=O)C1Cc2ccc(F)cc2CN1]

The compound was prepared by general procedure f with Uvi (3.52 g, 16.8 mmol) in 28-30% aqueous ammonium hydroxide (46.8 mL, 336 mmol), stirred at room temperature for 16 h. Uvii was obtained as a white solid white solid (2.83 g, 87%). ¹H NMR (CD₃OD, 400 MHz) δ 7.13 (dd, 1H), 6.87 (td, 1H), 6.82 (dd, 1H), 4.05 (d, 1H), 3.97 (d, 1H), 3.55 (dd, 1H), 3.02 (dd, 1H), 2.83 (dd, 1H).

### 7-Fluoro-2-methyl-1,2,3,4-tetrahydroisoquinoline-3-carboxamide (Uviii)

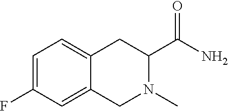

The compound was prepared by general procedure g1 with Uvii (0.8 g, 4.12 mmol), 37% aqueous formaldehyde (1.08 mL, 14.4 mmol) and sodium triacetoxyborohydride (3.49 g, 16.5 mmol) in acetonitrile (60 mL), stirred at room temperature for 5 h. Uviii was obtained as a white solid (0.786 g, 92%). ¹H NMR (CD₃OD, 300 MHz) δ 7.13 (dd, 1H), 6.90 (dd, 1H), 6.84 (d, 1H), 3.95 (d, 1H), 3.57 (d, 1H), 3.17 (dd, 1H), 3.10-2.92 (m, 2H), 2.44 (s, 3H).

### 1-(7-Fluoro-2-methyl-1,2,3,4-tetrahydroisoindolin-3-yl)methanamine (Uix)

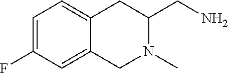

The compound was prepared by general procedure h with Uviii (0.386 g, 1.85 mmol) and lithium aluminium hydride (0.211 g, 5.56 mmol) in anhydrous tetrahydrofuran (36.9 mL), stirred at 60° C. for 18 h. After workup, the crude was concentrated to Uix as a yellow liquid (0.352 g, 98%). ¹H NMR (CDCl₃, 300 MHz) δ 6.82 (dd, 1H), 6.62 (td, 1H), 6.51 (dd, 1H), 3.58 (d, 1H), 3.43 (d, 1H), 2.68-2.37 (m, 5H), 2.16 (s, 3H).

### Methyl 2-amino-3-(4-chlorophenyl)propanoate hydrochloride (1:1) (Vii)

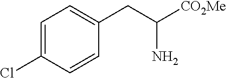 [SMILES: COC(=O)C(N)Cc1ccc(Cl)cc1]

The compound was prepared by general procedure a with dl-4-chlorophenylalanine Vi (3 g, 15 mmol) and thionyl chloride (1.2 mL, 16.5 mmol) in anhydrous methanol (75 mL), refluxed for 18 h. Vii hydrochloride (1:1) was obtained as a white solid (3.76 g, quantitative yield). ¹H NMR (300 MHz, d₆-DMSO) δ 8.74 (br s, 2H), 7.39 (d, 2H), 7.28 (d, 2H), 4.27 (t, 1H), 3.68 (s, 3H), 3.23-3.08 (m, 2H).

### Methyl 3-(4-chlorophenyl)-2-((ethoxycarbonyl)amino)propanoate (Viii)

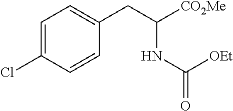 [SMILES: CCOC(=O)NC(Cc1ccc(Cl)cc1)C(=O)OC]

The compound was prepared by general procedure b2 with Vii hydrochloride (1:1) (3.46 g, 13.8 mmol), ethyl chloroformate (2.04 mL, 14.5 mmol) and pyridine (2.45 mL, 30.4 mmol) in dichloromethane (46 mL), stirred at 0° C. for 1 h. Viii was obtained as a white solid (3.89 g, 98%). ¹H NMR (300 MHz, d₆-DMSO) δ 7.63 (d, 1H), 7.34 (d, 2H), 7.26 (d, 2H), 4.26-4.18 (m, 1H), 3.92 (qd, 2H), 3.62 (s, 3H), 3.02 (dd, 1H), 2.84 (dd, 1H), 1.11 (t, 3H).

### 2-Ethyl 3-methyl 7-chloro-3,4-dihydroisoquinoline-2,3(1H)-dicarboxylate (Viv)

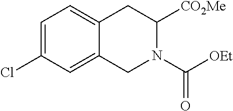 [SMILES: CCOC(=O)N1Cc2cc(Cl)ccc2CC1C(=O)OC]

The compound was prepared by general procedure c with Viii (3.89 g, 13.6 mmol) and paraformaldehyde (0.45 g, 15 mmol) in a mixture of acetic acid (40.5 mL) and sulfuric acid (13.5 mL), stirred at room temperature for 18 h. After workup, the crude was purified by flash column chromatography (SiO₂, 20% ethyl acetate in n-hexane) to give Viv as a colorless oil (3.09 g, 76%). ¹H NMR (300 MHz, CDCl₃) δ 7.27 (d, 1H), 7.15-7.06 (m, 2H), 5.29-4.94 (m, 1H), 4.78-4.39 (m, 2H), 4.29-4.14 (m, 2H), 3.62 (s, 3H), 3.27-3.07 (m, 2H), 1.35-1.23 (m, 3H).

### 7-Chloro-1,2,3,4-tetrahydroisoquinoline-3-carboxylic acid hydrochloride (1:1) (Vv)

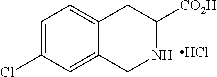 [SMILES: O=C(O)C1Cc2ccc(Cl)cc2CN1 ; Cl]

The compound was prepared by general procedure d with Viv (3.06 g, 13.6 mmol) in aqueous 6 N hydrogen chloride (51 mL), refluxed for 48 h. Vv hydrochloride (1:1) was obtained as a white solid (1.19 g, 84%). ¹H NMR (300 MHz, d₆-DMSO) δ 9.99 (br s, 1H), 8.45 (br s, 1H), 7.40-7.37 (m, 1H), 7.37-7.25 (m, 2H), 4.50-4.07 (m, 3H), 3.31 (dd overlapped with H₂O, 1H), 3.16-3.03 (m, 1H).

### Methyl 7-chloro-1,2,3,4-tetrahydroisoquinoline-3-carboxylate hydrochloride (1:1) (Vvi)

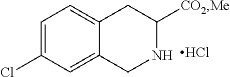

The compound was prepared by general procedure e2 with Vv hydrochloride (1.19 g, 4.8 mmol), thionyl chloride (1.22 mL, 16.8 mmol) and triethylamine (0.67 mL, 4.8 mmol) in anhydrous methanol (48 mL), refluxed for 18 h. Vvi hydrochloride (1:1) was obtained as a white solid (1.05 g, 83%). ¹H NMR (300 MHz, d₆-DMSO) δ 9.96 (br s, 2H), 7.40 (d, 1H), 7.38-7.27 (m, 2H), 4.54 (dd, 1H), 4.42-4.23 (m, 2H), 3.81 (s, 3H), 3.42-3.24 (m overlapped with H₂O, 1H), 3.11 (dd, 1H).

### 7-Chloro-1,2,3,4-tetrahydroisoquinoline-3-carboxamide (Vvii)

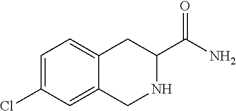 [SMILES: NC(=O)C1Cc2ccc(Cl)cc2CN1]

The compound was prepared by general procedure f with Vvi hydrochloride (1:1) (1.05 g, 4 mmol) in 28-30% aqueous ammonium hydroxide (40 mL), stirred at room temperature for 18 h. Vvii was obtained as a white solid (711 mg, 84%). ¹H NMR (300 MHz, d₆-DMSO) δ 7.33 (br s, 1H), 7.24-7.11 (m, 3H), 7.08 (br s, 1H), 4.01-3.76 (m, 2H), 3.39-3.27 (m overlapped with H₂O, 1H), 2.87 (dd, 1H), 2.67 (dd, 1H), 2.59 (br s, 1H).

### 7-Chloro-2-methyl-1,2,3,4-tetrahydroisoquinoline-3-carboxamide (Vviii)

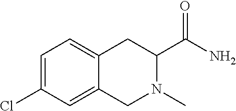

The compound was prepared by general procedure g1 with Vvii (711 mg, 3.38 mmol), 37% aqueous formaldehyde (0.96 mL, 11.8 mmol), sodium triacetoxyborohydride (2.86 g, 13.5 mmol) and acetic acid (0.2 mL, 3.38 mmol) in acetonitrile (54 mL) and tetrahydrofuran (13 mL), stirred at room temperature for 5 h. Vviii was obtained as a white solid (459 mg, 60%). ¹H NMR (300 MHz, d₆-DMSO) δ 7.41 (br s, 1H), 7.20-7.11 (m, 3H), 7.08 (br s, 1H), 3.87 (d, 1H), 3.50 (d, 1H), 3.10 (dd, 1H), 2.97-2.78 (m, 2H), 2.31 (s, 3H).

### (7-Chloro-2-methyl-1,2,3,4-tetrahydroisoquinolin-3-yl)methanamine (Vix)

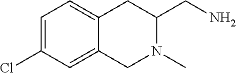

The compound was prepared by general procedure h with Vviii (459 mg, 2.04 mmol) and lithium aluminium hydride (232 mg, 6.13 mmol) in anhydrous tetrahydrofuran (70 mL), stirred at 60° C. for 4 h. After workup, the crude was purified by flash column chromatography (SiO₂, 10% methanol with 1% triethylamine in dichloromethane) to give Vix as a brown syrup (155 mg, 36%). ¹H NMR (300 MHz, d₆-DMSO) δ 7.19-6.99 (m, 3H), 3.74 (d, 1H), 3.51 (d, 1H), 2.74-2.68 (m, 3H), 2.57 (q, 1H), 2.52-2.43 (m overlapped with d₆-DMSO, 1H), 2.28 (s, 3H), 1.59 (br s, 1H).

### 2-(Prop-2-en-1-yl)-1,2,3,4-tetrahydroisoquinoline-3-carboxamide (Wviii)

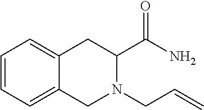 [SMILES: C=CCN1Cc2ccccc2CC1C(N)=O]

The compound was prepared by general procedure g2 with 1,2,3,4-tetrahydroisoquinoline-3-carboxamide Avii (300 g, 1.7 mmol), potassium carbonate (282 mg, 2.04 mmol), 3-bromopropene (155 μL, 1.79 mmol) in acetonitrile (17 mL) and tetrahydrofuran (17 mL), stirred at 60° C. for 18 h. After workup, the crude was purified by flash column chromatography (2% methanol in dichloromethane) to give Wviii as a white solid (277 mg, 75%). ¹H NMR (300 MHz, d₆-DMSO) δ 7.34 (br s, 1H), 7.14-7.09 (m, 3H), 7.09-7.00 (m, 2H), 5.90 (ddt, 1H), 5.26-5.10 (m, 2H), 3.91 (d, 1H), 3.58 (d, 1H), 3.38-3.33 (m overlapped with H₂O, 1H), 3.23 (dd, 1H), 3.11 (dd, 1H), 2.99-2.83 (m, 2H).

### (2-(Prop-2-en-1-yl)-1,2,3,4-tetrahydroisoquinolin-3-yl)methanamine (Wix)

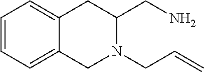

The compound was prepared by general procedure h with Wviii (277 mg, 1.28 mmol) and lithium aluminium hydride (146 mg, 3.84 mmol) in tetrahydrofuran (25.6 mL), stirred at 50° C. for 4 h. After workup, the crude was used for the next step without further purification. 2-(3-Methylbut-2-en-1-yl)-1,2,3,4-tetrahydroisoquinoline-3-carboxamide (Xviii)

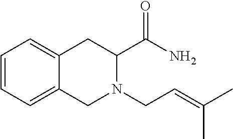 [SMILES: CC(C)=CCN1Cc2ccccc2CC1C(N)=O]

The compound was prepared by general procedure g3 with Avii (300 g, 1.7 mmol), potassium carbonate (282 mg, 2.04 mmol), 4-bromo-2-methyl-2-butene (210 μL, 1.79 mmol) in acetonitrile (8.5 mL) and tetrahydrofuran (8.5 mL), stirred at 60° C. for 4 h. After workup, the crude was purified by flash column chromatography (2% methanol in dichloromethane) to give Xviii as a white solid (306 mg, 74%). ¹H NMR (300 MHz, d₆-DMSO) δ 7.31 (br s, 1H), 7.15-7.08 (m, 3H), 7.08-7.01 (m, 2H), 5.28 (t, 1H), 3.90 (d, 1H), 3.54 (d, 1H), 3.37-3.32 (m overlapped with H₂O, 1H), 3.18 (dd, 1H), 3.04 (dd, 1H), 3.00-2.81 (m, 2H), 1.71 (s, 3H), 1.57 (s, 3H).

### (2-(3-Methylbut-2-en-1-yl)-1,2,3,4-tetrahydroisoquinolin-3-yl)methanamine (Xix)

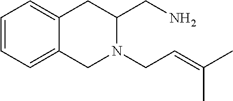 [SMILES: CC(C)=CCN1Cc2ccccc2CC1CN]

The compound was prepared by general procedure h with Xviii (306 mg, 1.25 mmol) and lithium aluminium hydride (143 mg, 3.76 mmol) in tetrahydrofuran (25 mL), stirred at 50° C. for 3 h. After workup, the crude was used for the next step without further purification.

### 2-(2-Methoxyethyl)-1,2,3,4-tetrahydroisoquinoline-3-carboxamide (Yviii)

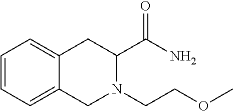 [SMILES: COCCN1Cc2ccccc2CC1C(N)=O]

The compound was prepared by general procedure g4 with Avii (0.1 g, 0.567 mmol), 2-bromoethyl methyl ether (82.3 μL, 0.851 mmol) and potassium carbonate (0.235 g, 1.7 mmol) in acetonitrile (2.8 mL), refluxed for 19 h. Yviii was obtained as a yellow solid (0.126 g, 95%). ¹H NMR (CDCl₃, 400 MHz) δ 7.56 (br s, 1H), 7.22-7.14 (m, 3H), 7.09 (dd, 1H), 5.47 (br s, 1H), 3.97 (d, 1H), 3.85 (d, 1H), 3.57-3.48 (m, 3H), 3.37 (s, 3H), 3.14 (dd, 1H), 3.03 (dd, 1H), 2.85-2.68 (m, 2H).

### 1-[2-(2-Methoxyethyl)-1,2,3,4-tetrahydroisoquinolin-3-yl]methanamine (Yix)

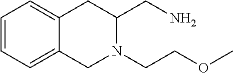 [SMILES: COCCN1Cc2ccccc2CC1CN]

The compound was prepared by general procedure h with Yviii (0.364 g, 1.55 mmol) and lithium aluminium hydride (0.177 g, 4.66 mmol) in tetrahydrofuran (30.9 mL), refluxed for 6 h. After workup, the crude was concentrated to give Yix as a yellow liquid (0.259 g, 76%). ¹H NMR (CDCl₃, 300 MHz) δ 7.01-6.83 (m, 4H), 3.78 (d, 1H), 3.69 (d, 1H), 3.45-3.27 (m, 2H), 3.20 (s, 3H), 2.86-2.76 (m, 1H), 2.70-2.38 (m, 6H).

### Methyl-2-amino-3-(2-methylphenyl)propanoate hydrochloride (Zii)

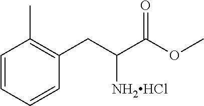

The compound was prepared by general procedure a with 2-amino-3-(2-methylphenyl)-propanoic acid Zi (5 g, 27.9 mmol) and thionyl chloride (4.05 mL, 55.8 mmol) in anhydrous methanol (130 mL), stirred at 50° C. for 17 h. Zii was obtained as a white solid (6.4 g, 99%). ¹H NMR (400 MHz, DMSO-d₆) δ 8.74 (br s, 3H), 7.24-7.05 (m, 4H), 4.09 (dd, 1H), 3.57 (s, 3H), 3.20 (dd, 1H), 3.03 (dd, 1H), 2.27 (s, 3H).

### Methyl-2-((ethoxycarbonyl)amino)-3-(2-methylphenyl)propanoate (Ziii)

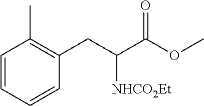

The compound was prepared by general procedure b2 with Zii (6.4 g, 27.9 mmol), ethyl chloroformate (4.11 mL, 29.3 mmol) and pyridine (4.94 mL, 61.3 mmol) in dichloromethane (130 mL), stirred at 0° C. for 2 h. After workup, the crude was purified by Isco Combi-Flash Companion column chromatography (0-20% ethyl acetate in n-hexane) to give Ziii (7.13 g, 97%). ¹H NMR (400 MHz, DMSO-d₆) δ 7.67 (d, 1H), 7.19-7.06 (m, 4H), 4.27-4.14 (m, 1H), 3.98-3.85 (m, 2H), 3.61 (s, 3H), 3.04 (dd, 1H), 2.84 (dd, 1H), 2.28 (d, 3H), 1.11 (t, 3H).

### 2-Ethyl 3-methyl-5-methyl-3,4-dihydroisoquinoline-2,3(1H)-dicarboxylate (Ziv)

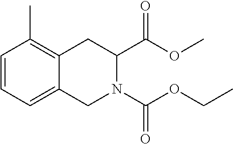 [SMILES: CCOC(=O)N1Cc2cccc(C)c2CC1C(=O)OC]

The compound was prepared by general procedure c with Ziii (7.13 g, 26.9 mmol) and paraformaldehyde (0.89 g, 29.6 mmol) in a mixture of acetic acid (70 mL) and sulfuric acid (30 mL), stirred at room temperature for 17 h. After workup, the crude was purified by Isco Combi-Flash Companion column chromatography (0-30% ethyl acetate in n-hexane) to give Ziv as a colorless liquid (5.53 g, 74%). ¹H NMR (400 MHz, DMSO-d₆) δ 7.28-6.88 (m, 3H), 5.10-4.85 (m, 1H), 4.61 (dd, 1H), 4.42 (dd, 1H), 4.22-4.04 (m, 2H), 3.61-3.50 (m, 3H), 3.16 (td, 1H), 3.00 (td, 1H), 2.22 (s, 3H), 1.24 (t, 1.5H), 1.16 (t, 1.5H).

### 5-Methyl-1,2,3,4-tetrahydroisoquinoline-3-carboxylic acid hydrochloride (1:1) (Zv)

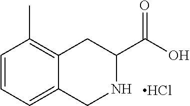

The compound was prepared by general procedure d with Ziv (5.53 g, 19.9 mmol) in aqueous 8 N hydrogen chloride (100 mL), refluxed for 17 h. Zv hydrochloride (1:1) was obtained as a white solid (2.55 g, 56%). ¹H NMR (400 MHz, DMSO-d₆) δ 9.75 (br s, 2H), 7.22-7.12 (m, 2H), 7.09 (dd, 1H), 4.40 (dd, 1H), 4.31 (s, 2H), 3.21 (dd, 1H), 2.91 (dd, 1H), 2.23 (s, 3H).

### Methyl 5-methyl-1,2,3,4-tetrahydroisoquinoline-3-carboxylate (Zvi)

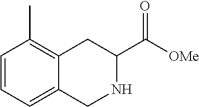

The compound was prepared by general procedure e2 with Zv hydrochloride (1:1) (2.55 g, 11.2 mmol) and thionyl chloride (2.84 mL, 39.2 mmol) in anhydrous methanol (45 mL), stirred at 50° C. for 17 h. After workup, the crude was concentrated to give Zvi (2.2 g, 96%). ¹H NMR (600 MHz, DMSO-d₆) δ 7.04-6.96 (m, 2H), 6.86 (d, 1H), 3.91 (d, 1H), 3.87 (d, 1H), 3.70-3.66 (m, 4H), 2.82 (dd, 1H), 2.62 (dd, 1H), 2.17 (s, 3H).

### 5-Methyl-1,2,3,4-tetrahydroisoquinoline-3-carboxamide (Zvii)

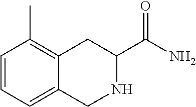

The compound was prepared by general procedure f with Zvi (2.2 g, 10.7 mmol) in 28-30% aqueous ammonium hydroxide (100 mL), stirred at room temperature for 17 h, to give Zvii (1.65 g, 81%). ¹H NMR (600 MHz, DMSO-d₆) δ 7.33 (br s, 1H), 7.11 (br s, 1H), 7.04-6.95 (m, 2H), 6.86 (d, 1H), 3.89 (d, 1H), 3.85 (d, 1H), 3.37-3.34 (m overlapped with H₂O, 1H), 2.79 (dd, 1H), 2.55-2.50 (m overlapped with DMSO-d₆, 1H), 2.17 (s, 3H).

### 2,5-Dimethyl-1,2,3,4-tetrahydroisoquinoline-3-carboxamide (Zviii)

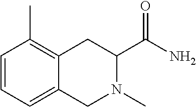

The compound was prepared by general procedure g1 with Zvii (1.65 g, 8.67 mmol), 37% aqueous formaldehyde (0.97 mL, 13 mmol), sodium triacetoxyborohydride (3.68 g, 17.3 mmol) and acetic acid (0.5 mL, 8.67 mmol) in acetonitrile (60 mL) and tetrahydrofuran (60 mL), stirred at room temperature for 2 h. After workup, the crude was purified by Isco Combi-Flash Companion column chromatography (0-5% methanol in CH₂Cl₂) to give Zviii (1.56 g, 88%). ¹H NMR (600 MHz, DMSO-d₆) δ 7.40 (br s, 1H), 7.10 (br s, 1H), 7.06-6.96 (m, 2H), 6.88 (d, 1H), 3.84 (d, 1H), 3.46 (d, 1H), 3.03 (dd, 1H), 2.77 (dd, 1H), 2.71 (dd, 1H), 2.29 (s, 3H), 2.16 (s, 3H).

### (2,5-Dimethyl-1,2,3,4-tetrahydroisoquinolin-3-yl)methanamine (Zix)

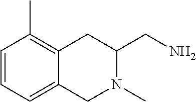

The compound was prepared by general procedure h with Zviii (0.3 g, 1.47 mmol) and 2M lithium aluminium hydride in THF (2.2 mL) in anhydrous tetrahydrofuran (15 mL), stirred at 60° C. for 17 h. After workup, the crude was purified by Isco Combi-Flash Companion column chromatography (0-5% methanol with 1% ammonia hydroxide in CH₂Cl₂) to give Zix (0.23 g, 82%). ¹H NMR (400 MHz, DMSO-d₆) δ 7.04-6.94 (m, 2H), 6.90-6.80 (m, 1H), 3.73 (d, 1H), 3.50 (d, 1H), 2.75 (dd, 1H), 2.66 (dd, 1H), 2.62-2.54 (m, 2H), 2.49-2.43 (m, 1H), 2.28 (s, 3H), 2.18 (s, 3H).

### Methyl (2S)-2-amino-3-(2-chlorophenyl)propanoate hydrochloride (aii)

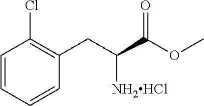

The compound was prepared by general procedure a with (2S)-2-amino-3-(2-chlorophenyl)-propanoic acid ai (4.3 g, 21.5 mmol) and thionyl chloride (3.13 mL, 43.1 mmol) in anhydrous methanol (100 mL), stirred at 50° C. for 17 h. Compound aii was obtained as a white solid (5.38 g, 99%). ¹H NMR (400 MHz, DMSO-d₆) δ 8.64 (br s, 3H), 7.52-7.43 (m, 1H), 7.42-7.37 (m, 1H), 7.37-7.30 (m, 2H), 4.19 (dd, 1H), 3.62 (s, 3H), 3.29-3.22 (m, 2H).

### Methyl (2S)-3-(2-chlorophenyl)-2-((ethoxycarbonyl)amino)propanoate (aiii)

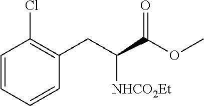

The compound was prepared by general procedure b2 with aii (5.38 g, 22.6 mmol), ethyl chloroformate (3.17 mL, 29.3 mmol) and pyridine (3.81 mL, 47.3 mmol) in dichloromethane (100 mL), stirred at 0° C. for 2 h. After workup, the crude was purified by Isco Combi-Flash Companion column chromatography (0-5% methanol in dichloromethane) to give aiii (6.1 g, 99%). ¹H NMR (400 MHz, DMSO-d₆) δ 7.69 (d, 1H), 7.46-7.39 (m, 1H), 7.39-7.31 (m, 1H), 7.30-7.23 (m, 2H), 4.34 (ddd, 1H), 3.99-3.84 (m, 2H), 3.62 (s, 3H), 3.22 (dd, 1H), 2.94 (dd, 1H), 1.10 (t, 3H).

### 2-Ethyl 3-methyl (3S)-5-chloro-3,4-dihydroisoquinoline-2,3(1H)-dicarboxylate (aiv)

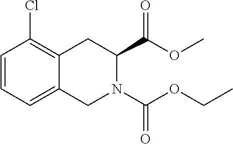 [SMILES: CCOC(=O)N1Cc2cccc(Cl)c2C[C@H]1C(=O)OC]

The compound was prepared by general procedure c with aiii (6.1 g, 21.3 mmol) and paraformaldehyde (0.71 g, 23.5 mmol) in a mixture of acetic acid (60 mL) and sulfuric acid (20 mL), stirred at room temperature for 17 h. After workup, the crude was purified by Isco Combi-Flash Companion column chromatography (0-5% methanol in dichloromethane) to give aiv as a colorless liquid (4.11 g, 65%). ¹H NMR (600 MHz, DMSO-d₆) δ 7.40-7.31 (m, 1H), 7.28-7.18 (m, 2H), 5.08 (ddd, 1H), 4.68 (dd, 1H), 4.44 (dd, 1H), 4.22-4.00 (m, 2H), 3.61-3.52 (m, 3H), 3.39 (dd, 1H), 3.08 (td, 1H), 1.25 (t, 1.5H), 1.17 (t, 1.5H).

### (3S)-5-Chloro-1,2,3,4-tetrahydroisoquinoline-3-carboxylic acid hydrochloride (1:1) (av)

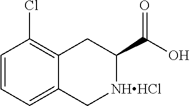

The compound was prepared by general procedure d with aiv (4.11 g, 13.8 mmol) in aqueous 8 N hydrogen chloride (90 mL), refluxed for 17 h. The desired product av was obtained as a white solid (2.98 g, 87%). ¹H NMR (400 MHz, DMSO-d₆) δ 10.06 (br s, 2H), 7.45 (dd, 1H), 7.37-7.24 (m, 2H), 4.47 (dd, 1H), 4.39 (d, 1H), 4.32 (d, 1H), 3.33 (dd, 1H), 3.01 (dd, 1H).

### Methyl (3S)-5-chloro-1,2,3,4-tetrahydroisoquinoline-3-carboxylate (avi)

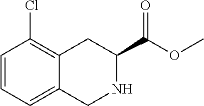

The compound was prepared by general procedure e2 with av (2.98 g, 12 mmol) and thionyl chloride (3.05 mL, 42 mmol) in anhydrous methanol (60 mL), stirred at 50° C. for 17 h. After workup, the crude was concentrated to give avi (2.49 g, 92%). ¹H NMR (400 MHz, DMSO-d₆) δ 7.27 (dd, 1H), 7.16 (t, 1H), 7.05 (dd, 1H), 3.95 (d, 1H), 3.88 (d, 1H), 3.74 (dd, 1H), 3.69 (s, 3H), 2.95 (dd, 1H), 2.85 (br s, 1H), 2.71 (dd, 1H).

### (3S)-5-Chloro-1,2,3,4-tetrahydroisoquinoline-3-carboxamide (avii)

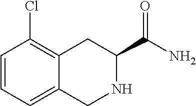 [SMILES: NC(=O)[C@@H]1Cc2c(Cl)cccc2CN1]

The compound was prepared by general procedure f with avi (2.49 g, 11 mmol) in 28-30% aqueous ammonium hydroxide (110 mL), stirred at room temperature for 17 h, to give avii (2.04 g, 88%). ¹H NMR (600 MHz, DMSO-d₆) δ 7.37 (br s, 1H), 7.26 (dd, 1H), 7.18-7.10 (m, 2H), 7.04 (dd, 1H), 3.93 (d, 1H), 3.86 (d, 1H), 3.41 (dd, 1H), 2.92 (dd, 1H), 2.60 (dd, 1H).

### (3S)-5-Chloro-2-methyl-1,2,3,4-tetrahydroisoquinoline-3-carboxamide (aviii)

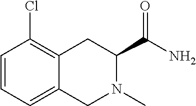

The compound was prepared by general procedure g1 with avii (2.04 g, 9.68 mmol), 37% aqueous formaldehyde (1.08 mL, 14.5 mmol), sodium triacetoxyborohydride (4.10 g, 19.4 mmol) and acetic acid (0.55 mL, 9.68 mmol) in acetonitrile (60 mL) and tetrahydrofuran (60 mL), stirred at room temperature for 2 h. After workup, the crude was purified by Isco Combi-Flash Companion column chromatography (0-5% methanol in CH₂Cl₂) to give aviii (2 g, 92%). ¹H NMR (400 MHz, DMSO-d₆) δ 7.46 (br s, 1H), 7.27 (d, 1H), 7.20-7.11 (m, 2H), 7.06 (d, 1H), 3.89 (d, 1H), 3.54 (d, 1H), 3.17 (dd, 1H), 2.90-2.80 (m, 2H), 2.31 (s, 3H). ((3S)-5-chloro-2-methyl-1,2,3,4-tetrahydroisoquinolin-3-yl)methanamine (aix)

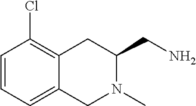

The compound was prepared by general procedure h with aviii (0.4 g, 1.78 mmol) and 2M lithium aluminium hydride in THF (2.67 mL, 5.34 mmol) in anhydrous tetrahydrofuran (17 mL), stirred at 60° C. for 17 h. After workup, the crude was purified by Isco Combi-Flash Companion column chromatography (0-5% methanol with 1% ammonia hydroxide in CH₂Cl₂) to give aix (0.22 g, 59%). ¹H NMR (400 MHz, DMSO-d₆) δ 7.27 (d, 1H), 7.15 (t, 1H), 7.05 (d, 1H), 3.78 (d, 1H), 3.54 (d, 1H), 2.83-2.59 (m, 4H), 2.58-2.46 (m overlapped with DMSO-d₆, 1H), 2.29 (s, 3H).

### Methyl (2S)-2-amino-3-(3-methylphenyl)propanoate hydrochloride (1:1) (bii)

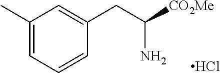

The compound was prepared by general procedure a with (2S)-2-amino-3-(3-methylphenyl)-propanoic acid bi (8 g, 44.6 mmol) and thionyl chloride (3.57 mL, 49.1 mmol) in anhydrous methanol (120 mL), refluxed for 18 h. The desired product bii hydrochloride (1:1) was obtained as a white solid (10.2 g, quantitative yield). ¹H NMR (600 MHz, d₆-DMSO) δ 8.50 (br s, 2H), 7.22 (t, 1H), 7.10 (d, 1H), 7.04 (s, 1H), 7.01 (d, 1H), 4.26 (t, 1H), 3.67 (s, 3H), 3.10 (dd, 1H), 3.04 (dd, 1H), 2.29 (s, 3H).

### Methyl (2S)-2-((ethoxycarbonyl)amino)-3-(3-methylphenyl)propanoate (biii)

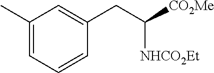

The compound was prepared by general procedure b2 with bii hydrochloride (1:1) (10.2 g, 44.6 mmol), ethyl chloroformate (4.49 mL, 46.9 mmol) and pyridine (7.91 mL, 98.2 mmol) in dichloromethane (153 mL), stirred at 0° C. for 1 h. The desired product biii was obtained as a brown oil (11.8 g, quantitative yield). ¹H NMR (600 MHz, d₆-DMSO) δ 7.62 (d, 1H), 7.16 (t, 1H), 7.06-7.00 (m, 3H), 4.19 (ddd, 1H), 3.97-3.88 (m, 2H), 3.61 (s, 3H), 2.96 (dd, 1H), 2.80 (dd, 1H), 2.26 (s, 3H), 1.11 (t, 3H).

### 2-Ethyl 3-methyl (3S)-6-methyl-3,4-dihydroisoquinoline-2,3(1H)-dicarboxylate (biv)

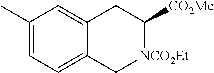

The compound was prepared by general procedure c with biii (11.8 g, 44.6 mmol) and paraformaldehyde (1.47 g, 49.1 mmol) in a mixture of acetic acid (66.8 mL) and sulfuric acid (22.2 mL), stirred at room temperature for 18 h. After workup, the crude was purified by flash column chromatography (SiO₂, 20% ethyl acetate in n-hexane) to give biv as a colorless oil (8.18 g, 66%). ¹H NMR (400 MHz, d₆-DMSO) δ 7.13-6.89 (m, 3H), 5.06-4.80 (m, 1H), 4.65-4.48 (m, 1H), 4.47-4.24 (m, 1H), 4.20-3.99 (m, 2H), 3.53 (s, 1.5H), 3.52 (s, 1.5H), 3.15-3.05 (m, 2H), 2.25-2.14 (m, 3H), 1.27-1.11 (m, 3H).

### (3S)-6-Methyl-1,2,3,4-tetrahydroisoquinoline-3-carboxylic acid hydrochloride (1:1) (bv)

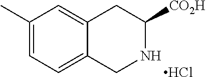

The compound was prepared by general procedure d with biv (8.17 g, 29.5 mmol) in aqueous 8 N hydrogen chloride (118 mL), refluxed for 48 h. The desired product bv hydrochloride (1:1) was obtained as a white solid (3.59 g, 54%). ¹H NMR (600 MHz, d₆-DMSO) δ 14.14 (br s, 1H), 9.77 (br s, 1H), 7.13 (d, 1H), 7.09-7.06 (m, 2H), 4.37 (dd, 1H), 4.31-4.22 (m, 2H), 3.25 (dd, 1H), 3.08 (dd, 1H), 2.27 (s, 3H).

### Methyl (3S)-6-methyl-1,2,3,4-tetrahydroisoquinoline-3-carboxylate (bvi)

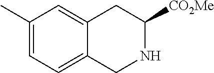

The compound was prepared by general procedure e2 with bv hydrochloride (1:1) (3.57 g, 15.7 mmol), thionyl chloride (3.99 mL, 54.9 mmol) and triethylamine (2.40 mL, 17.2 mmol) in anhydrous methanol (157 mL), refluxed for 18 h. The crude was purified by flash column chromatography (SiO₂, 0.5-1% methanol in dichloromethane) to give bvi as a colorless oil (1.44 g, 45%). ¹H NMR (600 MHz, d₆-DMSO) δ 6.93-6.89 (m, 3H), 3.90 (d, 1H), 3.83 (d, 1H), 3.67-3.62 (m, 4H), 2.89 (dd, 1H), 2.77 (dd, 1H), 2.71 (br s, 1H), 2.23 (s, 3H).

### (3S)-6-Methyl-1,2,3,4-tetrahydroisoquinoline-3-carboxamide (bvii)

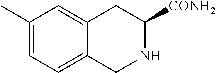

The compound was prepared by general procedure f with bvi (1.43 g, 7 mmol) in 28-30% aqueous ammonium hydroxide (140 mL), stirred at room temperature for 18 h. The desired product bvii was obtained as a white solid (803 mg, 60%). ¹H NMR (600 MHz, d₆-DMSO) δ 7.31 (br s, 1H), 7.05 (br s, 1H), 6.92-6.88 (m, 3H), 3.87 (d, 1H), 3.81 (d, 1H), 2.83 (dd, 1H), 2.67 (dd, 1H), 2.53-2.51 (m overlapped with DMSO, 1H), 2.23 (s, 3H).

### (3S)-2,6-Dimethyl-1,2,3,4-tetrahydroisoquinoline-3-carboxamide (bviii)

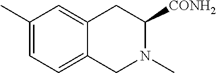

The compound was prepared by general procedure g1 with bvii (803 mg, 4.22 mmol), 37% aqueous formaldehyde (0.51 mL, 6.33 mmol), sodium triacetoxyborohydride (1.79 g, 8.44 mmol) and acetic acid (0.240 mL, 4.22 mmol) in tetrahydrofuran (21 mL), stirred at room temperature for 5 h. The desired product bviii was obtained as a white solid (813 mg, 94%). ¹H NMR (400 MHz, d₆-DMSO) δ 7.37 (br s, 1H), 7.04 (br s, 1H), 6.94-6.89 (m, 3H), 3.83 (d, 1H), 3.42 (d, 1H), 3.01 (dd, 1H), 2.89 (dd, 1H), 2.79 (dd, 1H), 2.30 (s, 3H), 2.23 (s, 3H).

### ((3S)-2,6-Dimethyl-1,2,3,4-tetrahydroisoquinolin-3-yl)methanamine (bix)

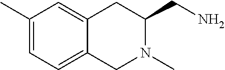

The compound was prepared by general procedure h with bviii (150 mg, 0.73 mmol) and 1 M lithium aluminium hydride solution in THF (2.2 mL, 2.2 mmol) in anhydrous tetrahydrofuran (15 mL), stirred at 60° C. for 18 h. After workup, the crude was purified by flash column chromatography (SiO₂, 20% methanol with 1% triethylamine in dichloromethane) to give bix as a yellow crystal (110 mg, 81%). ¹H NMR (600 MHz, d₆-DMSO) δ 6.92-6.89 (m, 2H), 6.88 (s, 1H), 3.68 (d, 1H), 3.46 (d, 1H), 2.72-2.66 (m, 3H), 2.55 (dd, 1H), 2.47-2.43 (m, 1H), 2.28 (s, 3H), 2.23 (s, 3H), 1.43 (br s, 2H).

### 6-Methoxy-1,2,3,4-tetrahydroisoquinoline-3-carboxylic acid hydrochloride (1:1) (cv)

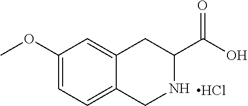

The compound was prepared by general procedure j1 with 2-amino-3-(3-methoxyphenyl) propanoic acid ci (1.5 g, 7.68 mmol) and formaldehyde (2.29 mL, 30.7 mmol) in aqueous 6 M hydrogen chloride (2.5 mL), stirred at room temperature for 18 h. The desired product cv hydrochloride (1:1) was obtained as a white solid (1.30 g, 69%). ¹H NMR (400 MHz, DMSO-d₆) δ 9.67 (br s, 2H), 7.18 (d, 1H), 6.92-6.80 (m, 2H), 4.38 (d, 1H), 4.29 (d, 1H), 4.22 (d, 1H), 3.74 (s, 3H), 3.29 (dd, 1H), 3.19-3.00 (m, 1H).

### Methyl 6-methoxy-1,2,3,4-tetrahydroisoquinoline-3-carboxylate hydrochloride (1:1) (cvi)

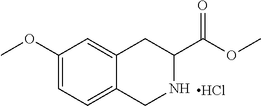

The compound was prepared by general procedure e2 with cv hydrochloride (1:1) (1.44 g, 5.91 mmol), thionyl chloride (1.5 mL, 20.7 mmol) and triethylamine (0.91 mL, 6.5 mmol) in anhydrous methanol (60 mL), stirred at 50° C. for 17 h. The desired product cvi hydrochloride (1:1) was obtained as a as a white solid (0.920 g, 77%). ¹H NMR (400 MHz, DMSO-d₆) δ 6.95 (dd, 1H), 6.74-6.67 (m, 2H), 3.89 (d, 1H), 3.81 (d, 1H), 3.71 (s, 3H), 3.69-3.63 (m, 4H), 2.93 (dd, 1H), 2.80 (dd, 1H).

### 6-Methoxy-1,2,3,4-tetrahydroisoquinoline-3-carboxamide (cvii)

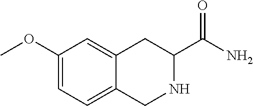

The compound was prepared by general procedure f with cvi hydrochloride (1.29 g, 5.83 mmol) in 28-30% aqueous ammonium hydroxide (30 mL), stirred at room temperature for 18 h. The desired product cvii was obtained as a white solid (0.920 g, 77%). ¹H NMR (400 MHz, DMSO-d₆) δ 7.31 (br s, 1H), 7.06 (br s, 1H), 6.93 (d, 1H), 6.73-6.63 (m, 2H), 3.85 (d, 1H), 3.78 (d, 1H), 3.70 (s, 3H), 3.36-3.28 (m overlapped with DMSO-d₆, 1H), 2.85 (dd, 1H), 2.69 (dd, 1H).

### 6-Methoxy-2-methyl-1,2,3,4-tetrahydroisoquinoline-3-carboxamide (cviii)

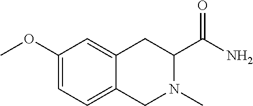 [SMILES: COc1ccc2c(c1)CC(C(N)=O)N(C)C2]

The compound was prepared by general procedure g1 with cvii (0.92 g, 4.46 mmol), 37% aqueous formaldehyde (0.5 mL, 6.69 mmol), sodium triacetoxyborohydride (1.89 g, 8.92 mmol) and acetic acid (0.25 mL, 4.46 mmol) in acetonitrile (20 mL) and tetrahydrofuran (20 mL), stirred at room temperature for 1 h. The desired product cviii was obtained as a white solid (0.880 g, 90%). ¹H NMR (400 MHz, DMSO-d₆) δ 7.37 (br s, 1H), 7.05 (br s, 1H), 6.96 (d, 1H), 6.73-6.66 (m, 2H), 3.81 (d, 1H), 3.70 (s, 3H), 3.40 (d, 1H), 3.00 (dd, 1H), 2.91 (dd, 1H), 2.81 (dd, 1H), 2.30 (s, 3H).

### (6-Methoxy-2-methyl-1,2,3,4-tetrahydroisoquinolin-3-yl)methanamine (cix)

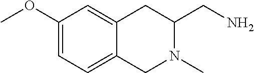

The compound was prepared by general procedure h with cviii (0.3 g, 1.36 mmol) and 2M lithium aluminium hydride in THF (2.04 mL, 4.09 mmol) in anhydrous tetrahydrofuran (15 mL), stirred at 60° C. for 17 h. After workup, the crude was purified by Isco Combi-Flash Companion column chromatography (0-5% methanol with 1% ammonia hydroxide in CH₂Cl₂) to give cix as a yellow oil (0.210 g, 75%). ¹H NMR (400 MHz, DMSO-d₆) δ 6.94 (d, 1H), 6.68 (d, 1H), 6.66 (s, 1H), 3.71 (s, 3H), 3.67 (d, 1H), 3.44 (d, 1H), 2.76-2.68 (m, 3H), 2.57 (dd, 1H), 2.48-2.41 (m, 1H), 2.29 (s, 3H).

### (3S)-6-Methoxy-1,2,3,4-tetrahydroisoquinoline-3-carboxylic acid hydrochloride (1:1) (dv)

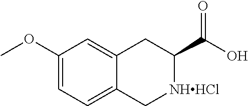

The compound was prepared by general procedure j1 with (2S)-2-amino-3-(3-methoxyphenyl) propanoic acid di (2 g, 10.2 mmol) and formaldehyde (3.05 mL, 41 mmol) in aqueous 6 M hydrogen chloride (3.5 mL), stirred at room temperature for 18 h. The desired product dv hydrochloride (1:1) was obtained as a white solid (2.09 g, 84%). ¹H NMR (400 MHz, DMSO-d₆) δ 9.84 (br s, 2H), 7.17 (d, 1H), 6.91-6.83 (m, 2H), 4.38 (dd, 1H), 4.28 (d, 1H), 4.21 (d, 1H), 3.74 (s, 3H), 3.28 (dd, 1H), 3.11 (dd, 1H).

### Methyl (3S)-6-methoxy-1,2,3,4-tetrahydroisoquinoline-3-carboxylate hydrochloride (1:1) (dvi)

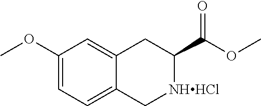

The compound was prepared by general procedure e2 with dv hydrochloride (1:1) (2.09 g, 8.58 mmol), thionyl chloride (2.18 mL, 30 mmol) and triethylamine (1.31 mL, 9.43 mmol) in anhydrous methanol (85 mL), stirred at 50° C. for 17 h. The desired product (1:1) dvi was obtained as a as a white solid (0.920 g, 77%). ¹H NMR (400 MHz, DMSO-d₆) δ 6.94 (d, 1H), 6.76-6.66 (m, 2H), 3.89 (d, 1H), 3.81 (d, 1H), 3.71 (s, 3H), 3.67 (s, 3H), 3.66-3.62 (m, 1H), 2.92 (dd, 1H), 2.81 (dd, 1H), 2.71 (br s, 1H).

### (3S)-6-Methoxy-1,2,3,4-tetrahydroisoquinoline-3-carboxamide (dvii)

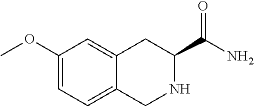

The compound was prepared by general procedure f with dvi (1.89 g, 8.54 mmol) in 28-30% aqueous ammonium hydroxide (60 mL), stirred at room temperature for 17 h. The desired product dvii was obtained as a white solid (1.04 g, 59%). ¹H NMR (400 MHz, DMSO-d₆) δ 7.31 (br s, 1H), 7.06 (br s, 1H), 6.93 (d, 1H), 6.74-6.64 (m, 2H), 3.85 (d, 1H), 3.78 (d, 1H), 3.70 (s, 3H), 3.36-3.28 (m overlapped with H₂O, 1H), 2.85 (dd, 1H), 2.69 (dd, 1H).

### (3S)-6-Methoxy-2-methyl-1,2,3,4-tetrahydroisoquinoline-3-carboxamide (dviii)

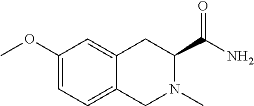 [SMILES: COc1ccc2c(c1)C[C@@H](C(N)=O)N(C)C2]

The compound was prepared by general procedure g1 with dvii (1.04 g, 5.04 mmol), 37% aqueous formaldehyde (0.56 mL, 7.56 mmol), sodium triacetoxyborohydride (2.14 g, 10.1 mmol) and acetic acid (0.29 mL, 5.04 mmol) in acetonitrile (20 mL) and tetrahydrofuran (20 mL), stirred at room temperature for 2 h. The desired product dviii was obtained as a white solid (1.10 g, 99%). ¹H NMR (400 MHz, DMSO-d₆) δ 7.38 (br s, 1H), 7.06 (br s, 1H), 6.97 (d, 1H), 6.74-6.68 (m, 2H), 3.81 (d, 1H), 3.71 (s, 3H), 3.41 (d, 1H), 3.01 (dd, 1H), 2.92 (dd, 1H), 2.82 (dd, 1H), 2.30 (s, 3H).

### ((3S)-6-Methoxy-2-methyl-1,2,3,4-tetrahydroisoquinolin-3-yl)methanamine (dix)

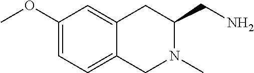

The compound was prepared by general procedure h with dviii (0.3 g, 1.36 mmol) and 2M lithium aluminium hydride in THF (2.04 mL, 4.09 mmol) in anhydrous tetrahydrofuran (15 mL), stirred at 60° C. for 17 h. After workup, the crude was purified by Isco Combi-Flash Companion column chromatography (0-5% methanol with 1% ammonia hydroxide in CH₂Cl₂) to give dix as a yellow oil (0.220 g, 78%). ¹H NMR (400 MHz, DMSO-d₆) δ 6.94 (d, 1H), 6.68 (d, 1H), 6.65 (s, 1H), 3.71 (s, 3H), 3.67 (d, 1H), 3.44 (d, 1H), 2.76-2.67 (m, 3H), 2.56 (dd, 1H), 2.49-2.40 (m, 1H), 2.29 (s, 3H), 1.45 (br s, 2H).

### Methyl (2S)-2-amino-3-(3-fluorophenyl)propanoate hydrochloride (1:1) (eii)

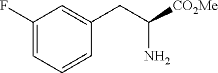 [SMILES: COC(=O)[C@@H](N)Cc1cccc(F)c1]

The compound was prepared by general procedure a with (2S)-2-amino-3-(3-fluorophenyl)-propanoic acid ei (5 g, 25.9 mmol) and thionyl chloride (2.18 mL, 30 mmol) in anhydrous methanol (75 mL), refluxed for 18 h. The desired product eii was obtained as a white solid (5.38 g, quantitative yield), and used to next step without purification.

### Methyl (2S)-2-((ethoxycarbonyl)amino)-3-(3-fluorophenyl)propanoate (eiii)

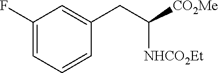

The compound was prepared by general procedure b2 with eii hydrochloride (1:1) (5.38 g, 25.9 mmol), ethyl chloroformate (2.60 mL, 27.2 mmol) and pyridine (4.60 mL, 56.9 mmol) in dichloromethane (250 mL), stirred at 0° C. for 1 h. The desired product eiii was obtained as a white solid (7.72 g, quantitative yield), and used to next step without purification.

### 2-Ethyl 3-methyl (3S)-6-fluoro-3,4-dihydroisoquinoline-2,3(1H)-dicarboxylate (eiv)

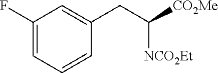

The compound was prepared by general procedure c with eiii (7.72 g, 25.9 mmol) and paraformaldehyde (902 mg, 30 mmol) in a mixture of acetic acid (39 mL) and sulfuric acid (13 mL), stirred at room temperature for 18 h. After workup, the crude was purified by flash column chromatography (SiO₂, 20% ethyl acetate in n-hexane) to give eiv as a yellow oil (7.67 g, quantitative yield). ¹H NMR (600 MHz, d₆-DMSO) δ 7.32-7.23 (m, 1H), 7.13-7.07 (m, 1H), 7.02 (td, 1H), 5.12-4.84 (m, 1H), 4.60 (dd, 1H), 4.43 (dd, 1H), 4.18-4.02 (m, 2H), 3.57 (s, 1.5H), 3.55 (s, 1.5H), 3.24-3.08 (m, 2H), 1.24 (t, 1.5H), 1.15 (t, 1.5H).

### (3S)-6-Fluoro-1,2,3,4-tetrahydroisoquinoline-3-carboxylic acid hydrochloride (1:1) (ev)

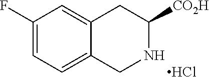 [SMILES: O=C(O)[C@@H]1Cc2cc(F)ccc2CN1 ; Cl]

The compound was prepared by general procedure d with eiv (7.67 g, 13.6 mmol) in aqueous 6 N hydrogen chloride (109 mL), refluxed for 48 h. The desired product ev hydrochloride (1:1) was obtained as a white solid (5.19 g, 87%). ¹H NMR (600 MHz, d₆-DMSO) δ 14.14 (br s, 1H), 9.97 (br s, 2H), 7.32 (dd, 1H), 7.17 (dd, 1H), 7.13 (td, 1H), 4.39 (dd, 1H), 4.33 (d, 1H), 4.25 (d, 1H), 3.33 (dd overlapped with H₂O, 1H), 3.14 (dd, 1H).

### Methyl (3S)-6-fluoro-1,2,3,4-tetrahydroisoquinoline-3-carboxylate (evi)

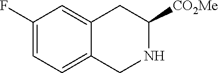 [SMILES: COC(=O)[C@@H]1Cc2cc(F)ccc2CN1]

The compound was prepared by general procedure e2 with ev hydrochloride (1:1) (5.19 g, 22.4 mmol), thionyl chloride (5.69 mL, 78.4 mmol) and triethylamine (3.12 mL, 22.4 mmol) in anhydrous methanol (75 mL), refluxed for 18 h. The desired product evi was obtained as a colorless oil (3.48 g, 74%). ¹H NMR (600 MHz, d₆-DMSO) δ 7.07 (dd, 1H), 6.98-6.92 (m, 2H), 3.92 (d, 1H), 3.84 (d, 1H), 3.69-3.64 (m, 4H), 2.95 (dd, 1H), 2.86-2.75 (m, 2H).

### (3S)-6-Fluoro-1,2,3,4-tetrahydroisoquinoline-3-carboxamide (evii)

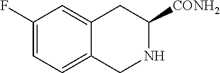 [SMILES: NC(=O)[C@@H]1Cc2cc(F)ccc2CN1]

The compound was prepared by general procedure f with evi (3.48 g, 16.6 mmol) in 28-30% aqueous ammonium hydroxide (166 mL), stirred at room temperature for 18 h. The desired product evii was obtained as a white solid (1.01 g, 34%). ¹H NMR (600 MHz, d₆-DMSO) δ 7.32 (br s, 1H), 7.11-7.03 (m, 2H), 6.98-6.90 (m, 2H), 3.90 (d, 1H), 3.82 (d, 1H), 3.36-3.33 (m overlapped with H₂O, 1H), 2.89 (dd, 1H), 2.72 (dd, 1H).

### (3S)-6-Fluoro-2-methyl-1,2,3,4-tetrahydroisoquinoline-3-carboxamide (eviii)

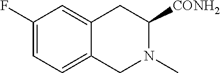

The compound was prepared by general procedure g1 with evii (1.09 g, 5.61 mmol), 37% aqueous formaldehyde (0.68 mL, 8.42 mmol), sodium triacetoxyborohydride (2.38 g, 11.2 mmol) and acetic acid (0.32 mL, 5.61 mmol) in tetrahydrofuran (56 mL), stirred at room temperature for 1 h. The desired product eviii was obtained as a white solid (1.16 g, quantitative yield). ¹H NMR (600 MHz, d₆-DMSO) δ 7.40 (br s, 1H), 7.12-7.05 (m, 2H), 6.99-6.92 (m, 2H), 3.85 (d, 1H), 3.46 (d, 1H), 3.06 (dd, 1H), 2.93 (dd, 1H), 2.86 (dd, 1H), 2.31 (s, 3H).

### ((3S)-6-Fluoro-2-methyl-1,2,3,4-tetrahydroisoquinolin-3-yl)methanamine (eix)

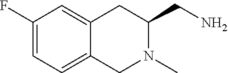

The compound was prepared by general procedure h with eviii (700 mg, 3.36 mmol) and 2M lithium aluminium hydride in THF (5.04 mL, 10.1 mmol) in anhydrous tetrahydrofuran (34 mL), stirred at 60° C. for 18 h. After workup, the crude was purified by flash column chromatography (SiO₂, 10% methanol with 1% triethylamine in dichloromethane) to give eix as a yellow oil (543 mg, 83%). ¹H NMR (600 MHz, d₆-DMSO) δ 7.09-7.04 (m, 1H), 6.94-6.89 (m, 2H), 3.72 (d, 1H), 3.47 (d, 1H), 2.74 (d, 2H), 2.71 (dd, 1H), 2.56 (dd, 1H), 2.48-2.43 (m, 1H), 2.29 (s, 3H), 1.41 (br s, 2H).

### Methyl (2S)-2-amino-3-(3-chlorophenyl)propanoate (fii)

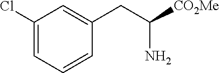 [SMILES: COC(=O)[C@@H](N)Cc1cccc(Cl)c1]

The compound was prepared by general procedure a with (2S)-2-amino-3-(3-chlorophenyl) propanoic acid fi (10 g, 50.1 mmol) and thionyl chloride (4 mL, 55.1 mmol) in anhydrous methanol (150 mL), refluxed for 18 h. The desired product fii was obtained as a white solid (3.66 g, 34%). ¹H NMR (600 MHz, d₆-DMSO) δ 7.31-7.28 (m, 1H), 7.28-7.25 (m, 2H), 7.15 (dt, 1H), 3.58 (s, 3H), 3.56 (dd, 1H), 2.87 (dd, 1H), 2.75 (dd, 1H), 1.79 (s, 2H).

### Methyl (2S)-3-(3-chlorophenyl)-2-((ethoxycarbonyl)amino)propanoate (fiii)

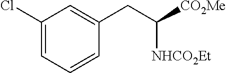

The compound was prepared by general procedure b2 with fii (3.66 g, 17.1 mmol), ethyl chloroformate (2.52 mL, 18 mmol) and pyridine (3.03 mL, 37.7 mmol) in dichloromethane (54.9 mL), stirred at 0° C. for 1 h. The desired product fiii was obtained as a yellow oil (4.89 g, quantitative yield). ¹H NMR (600 MHz, d₆-DMSO) δ 7.66 (d, 1H), 7.33 (br t, 1H), 7.31 (d, 1H), 7.28 (dt, 1H), 7.21 (dt, 1H), 4.24 (ddd, 1H), 3.98-3.87 (m, 2H), 3.63 (s, 3H), 3.04 (dd, 1H), 2.84 (dd, 1H), 1.10 (t, 3H).

### 2-Ethyl 3-methyl (3S)-6-chloro-3,4-dihydroisoquinoline-2,3(1H)-dicarboxylate (fiv)

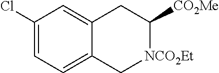

The compound was prepared by general procedure c with fiii (4.89 g, 17.1 mmol) and paraformaldehyde (566 mg, 18.8 mmol) in a mixture of acetic acid (25.5 mL) and sulfuric acid (8.5 mL), stirred at room temperature for 18 h. After workup, the crude was purified by flash column chromatography (SiO₂, 20% ethyl acetate in n-hexane) to give fiv as a colorless oil (3.61 g, 71%). ¹H NMR (600 MHz, d₆-DMSO) δ 7.38-7.32 (m, 1H), 7.30-7.21 (m, 2H), 5.12-4.85 (m, 1H), 4.70-4.56 (m, 1H), 4.43 (dd, 1H), 4.20-4.03 (m, 2H), 3.59-3.53 (m, 3H), 3.23-3.10 (m, 2H), 1.28-1.22 (m, 1.5H), 1.20-1.13 (m, 1.5H).

### (3S)-6-Chloro-1,2,3,4-tetrahydroisoquinoline-3-carboxylic acid hydrochloride (1:1) (fv)

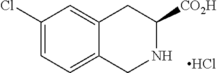 [SMILES: O=C(O)[C@@H]1Cc2cc(Cl)ccc2CN1 ; Cl]

The compound was prepared by general procedure d with fiv (3.61 g, 12.1 mmol) in aqueous 8 N hydrogen chloride (121 mL), refluxed for 48 h. The desired product fv was obtained as a white solid (2.18 g, 51%). ¹H NMR (600 MHz, d₆-DMSO) δ 9.81 (br s, 1H), 7.41 (d, 1H), 7.34 (dd, 1H), 7.30 (d, 1H), 4.41-4.32 (m, 2H), 4.26 (d, 1H), 3.33-3.24 (m overlapped with H₂O, 2H), 3.10 (dd, 1H).

### Methyl (3S)-6-chloro-1,2,3,4-tetrahydroisoquinoline-3-carboxylate (fvi)

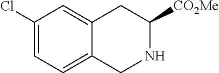 [SMILES: COC(=O)[C@@H]1Cc2cc(Cl)ccc2CN1]

The compound was prepared by general procedure e2 with fv hydrochloride (1:1) (2.18 g, 8.79 mmol), thionyl chloride (2.23 mL, 30.8 mmol) and triethylamine (1.35 mL, 9.66 mmol) in anhydrous methanol (44 mL), refluxed for 18 h. The desired product fvi was obtained as a colorless oil (720 mg, 36%). ¹H NMR (600 MHz, d₆-DMSO) δ 7.20 (d, 1H), 7.16 (dd, 1H), 7.07 (d, 1H), 3.92 (d, 1H), 3.84 (d, 1H), 3.69-3.65 (m, 4H), 2.95 (dd, 1H), 2.84-2.78 (m, 2H).

### (3S)-6-Chloro-1,2,3,4-tetrahydroisoquinoline-3-carboxamide (fvii)

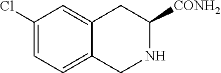 [SMILES: NC(=O)[C@@H]1Cc2cc(Cl)ccc2CN1]

The compound was prepared by general procedure f with fvi (720 mg, 3.19 mmol) in 28-30% aqueous ammonium hydroxide (32 mL), stirred at room temperature for 18 h. The desired product fvii was obtained as a white solid (520 mg, 77%). ¹H NMR (600 MHz, d₆-DMSO) δ 7.33 (br s, 1H), 7.19 (d, 1H), 7.15 (dd, 1H), 7.08 (br s, 1H), 7.06 (d, 1H), 3.90 (d, 1H), 3.82 (d, 1H), 3.38-3.32 (m, 1H), 2.89 (dd, 1H), 2.71 (dd, 1H), 2.59 (br s, 1H).

### (3S)-6-Chloro-2-methyl-1,2,3,4-tetrahydroisoquinoline-3-carboxamide (fviii)

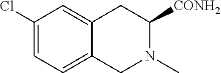

The compound was prepared by general procedure g1 with fvii (520 mg, 2.47 mmol), 37% aqueous formaldehyde (0.3 mL, 3.7 mmol), sodium triacetoxyborohydride (1.05 g, 4.94 mmol) and acetic acid (0.14 mL, 2.47 mmol) in tetrahydrofuran (12 mL), stirred at room temperature for 2 h. The desired product fviii was obtained as a white solid (599 mg, quantitative yield). ¹H NMR (400 MHz, d₆-DMSO) δ 7.41 (br s, 1H), 7.21 (d, 1H), 7.16 (dd, 1H), 7.11-7.05 (m, 2H), 3.86 (d, 1H), 3.47 (d, 1H), 3.07 (dd, 1H), 2.97-2.82 (m, 2H), 2.31 (s, 3H). ((3S)-6-Chloro-2-methyl-1,2,3,4-tetrahydroisoquinolin-3-yl)methanamine (fix)

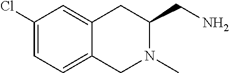

The compound was prepared by general procedure h with fviii (120 mg, 0.534 mmol) and 1M lithium aluminium hydride in THF (1.6 mL, 1.6 mmol) in anhydrous tetrahydrofuran (10.6 mL), stirred at 60° C. for 18 h. After workup, the crude was purified by flash column chromatography (SiO₂, 10-20% methanol with 1% triethylamine in dichloromethane) to give fix as a colorless oil (77 mg, 69%). ¹H NMR (600 MHz, d₆-DMSO) δ 7.16-7.13 (m, 2H), 7.07 (d, 1H), 3.72 (d, 1H), 3.48 (d, 1H), 2.74 (d, 2H), 2.70 (dd, 1H), 2.55 (dd, 1H), 2.49-2.45 (m overlapped with DMSO, 1H), 2.29 (s, 3H), 1.48 (br s, 2H).

### Methyl 2-amino-3-(3-bromophenyl)propanoate hydrochloride (1:1) (gii)

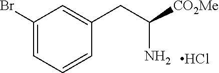 [SMILES: COC(=O)[C@@H](N)Cc1cccc(Br)c1 ; Cl]

The compound was prepared by general procedure a with 2-amino-3-(3-bromophenyl) propanoic acid gi (8 g, 32.8 mmol) and thionyl chloride (2.62 mL, 36 mmol) in anhydrous methanol (120 mL), refluxed for 18 h. The desired product gii hydrochloride (1:1) was obtained as a white solid (9.64 g, 99%). ¹H NMR (600 MHz, d₆-DMSO) δ 8.80-8.43 (br, 3H), 7.53-7.47 (m, 2H), 7.30 (t, 1H), 7.28-7.23 (m, 1H), 4.33 (t, 1H), 3.69 (s, 3H), 3.20-3.08 (m, 2H).

### Methyl 3-(3-bromophenyl)-2-((ethoxycarbonyl)amino)propanoate (giii)

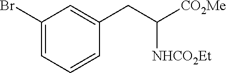

The compound was prepared by general procedure b2 with gii hydrochloride (1:1) (9.64 g, 32.7 mmol), ethyl chloroformate (3.3 mL, 34.4 mmol) and pyridine (5.8 mL, 72 mmol) in dichloromethane (95 mL), stirred at 0° C. for 1 h. The desired product giii was obtained as a yellow oil (11 g, quantitative yield). ¹H NMR (600 MHz, d₆-DMSO) δ 7.64 (d, 1H), 7.47 (br s, 1H), 7.43-7.40 (m, 1H), 7.29-7.23 (m, 2H), 4.24 (ddd, 1H), 3.97-3.89 (m, 2H), 3.63 (s, 3H), 3.04 (dd, 1H), 2.84 (dd, 1H), 1.11 (t, 3H).

### 2-Ethyl 3-methyl 6-bromo-3,4-dihydroisoquinoline-2,3(1H)-dicarboxylate (giv)

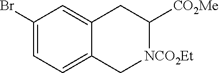

The compound was prepared by general procedure c with giii (11 g, 33.2 mmol) and paraformaldehyde (1.1 g, 36.6 mmol) in a mixture of acetic acid (99 mL) and sulfuric acid (33 mL), stirred at room temperature for 18 h. After workup, the crude was purified by flash column chromatography (SiO₂, 20% ethyl acetate in n-hexane) to give a mixture of giv and 2-ethyl 3-methyl 8-bromo-3,4-dihydroisoquinoline-2,3(1H)-dicarboxylate as a colorless oil (9.36 g, 82%). The ¹H NMR spectrum of giv is listed as follows. ¹H NMR (600 MHz, d₆-DMSO) δ 7.55-7.14 (m, 3H), 5.10-4.86 (m, 1H), 4.67-4.54 (m, 1H), 4.47-4.32 (m, 1H), 4.21-4.04 (m, 2H), 3.62-3.53 (m, 3H), 3.26-3.10 (m, 2H), 1.28-1.21 (m, 1.5H), 1.20-1.12 (m, 1.5H).

### 6-Bromo-1,2,3,4-tetrahydroisoquinoline-3-carboxylic acid hydrochloride (1:1) (gv)

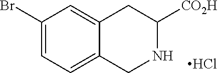 [SMILES: O=C(O)C1Cc2cc(Br)ccc2CN1 ; Cl]

The compound was prepared by general procedure d with giv (9.36 g, 27.4 mmol) in aqueous 8 N hydrogen chloride (273 mL), refluxed for 48 h. A mixture of 6-bromo-1,2,3,4-tetrahydroisoquinoline-3-carboxylic acid hydrochloride (1:1) gv and 8-bromo-1,2,3,4-tetrahydroisoquinoline-3-carboxylic acid was obtained as a white solid (7.35 g, 92%). The ¹H NMR spectrum of 6-bromo-1,2,3,4-tetrahydroisoquinoline-3-carboxylic acid hydrochloride (1:1) gv is listed. ¹H NMR (600 MHz, d₆-DMSO) δ 14.16 (br s, 1H), 9.88 (br s, 2H), 7.55 (d, 1H), 7.47 (dd, 1H), 7.23 (d, 1H), 4.39 (dd, 1H), 4.33 (d, 1H), 4.24 (d, 1H), 3.36-3.30 (m overlapped with H₂O, 1H), 3.12 (dd, 1H).

### Methyl 6-bromo-1,2,3,4-tetrahydroisoquinoline-3-carboxylate (gvi)

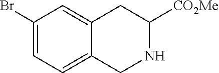 [SMILES: COC(=O)C1Cc2cc(Br)ccc2CN1]

The compound was prepared by general procedure e2 with gv hydrochloride (1:1) (7.35 g, 25.1 mmol), thionyl chloride (6.39 mL, 87.9 mmol) and triethylamine (3.50 mL, 25.1 mmol) in anhydrous methanol (150 mL), refluxed for 18 h. The crude was purified by flash column chromatography (SiO₂, 0.5-1% methanol in dichloromethane) to give gvi as a colorless oil (2.19 g, 32%). ¹H NMR (600 MHz, d₆-DMSO) δ 7.34 (d, 1H), 7.29 (dd, 1H), 7.01 (d, 1H), 3.90 (d, 1H), 3.82 (d, 1H), 3.68-3.65 (m, 4H), 2.95 (dd, 1H), 2.88-2.76 (m, 2H).

### Methyl 6-bromo-2-methyl-1,2,3,4-tetrahydroisoquinoline-3-carboxylate (gxv)

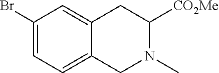 [SMILES: COC(=O)C1Cc2cc(Br)ccc2CN1C]

The compound was prepared by general procedure g1 with gvi (2.19 g, 8.11 mmol), 37% aqueous formaldehyde (0.987 mL, 12.2 mmol), sodium triacetoxyborohydride (3.44 g, 16.2 mmol) and acetic acid (0.463 mL, 8.11 mmol) in tetrahydrofuran (80 mL), stirred at room temperature for 2 h. After workup, the crude was purified by flash column chromatography (SiO₂, 2% methanol in dichloromethane) to give gxv as a white solid (1.39 g, 60%). ¹H NMR (400 MHz, d₆-DMSO) δ 7.35 (d, 1H), 7.30 (dd, 1H), 7.02 (d, 1H), 3.79 (d, 1H), 3.67-3.61 (m, 2H), 3.60 (s, 3H), 3.06 (dd, 1H), 2.96 (dd, 1H), 2.44 (s, 3H).

### (6-Bromo-2-methyl-1,2,3,4-tetrahydroisoquinolin-3-yl) methanol (gxvi)

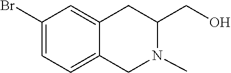

To a solution of gxv (1.39 mg, 4.89 mmol) in anhydrous tetrahydrofuran (40 mL) was added 1 M lithium triethylborohydride in THF (22 mL, 22 mmol), and the reaction mixture was stirred at room temperature for 18 h. After reaction completion, the crude mixture was quenched with saturated NH₄Cl_{(aq)}, then diluted with water, extracted with dichloromethane, then the organic layer was concentrated under reduced pressure. The crude was purified by flash column chromatography (SiO₂, 6% methanol in dichloromethane) to give gxvi as a white solid (980 mg, 78%). ¹H NMR (600 MHz, d₆-DMSO) δ 7.31 (d, 1H), 7.28 (dd, 1H), 7.01 (d, 1H), 4.58 (br s, 1H), 3.70 (d, 1H), 3.57 (dt, 1H), 3.44 (d, 1H), 3.37 (dt overlapped with H₂O, 1H), 2.78 (dd, 1H), 2.70 (dd, 1H), 2.55-2.51 (m overlapped with DMSO, 1H), 2.35 (s, 3H).

### (6-Bromo-2-methyl-1,2,3,4-tetrahydroisoquinolin-3-yl)methyl methanesulfonate (gxvii)

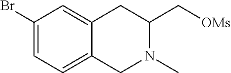 [SMILES: CN1Cc2ccc(Br)cc2CC1COS(C)(=O)=O]

To a solution of gxvi (980 mg, 3.83 mmol) in anhydrous dichloromethane (76 mL) at 0° C. was added methanesulfonyl chloride (0.38 mL, 4.97 mmol) and triethylamine (0.69 mL, 4.97 mmol), and the reaction mixture was stirred at room temperature for 2 h. After reaction completion, the crude mixture was diluted with dichloromethane, extracted with saturated NaHCO_{3(aq)}, and the organic layer was concentrated under reduced pressure. The crude was used for the next step without further purification.

### 3-((6-Bromo-2-methyl-1,2,3,4-tetrahydroisoquinolin-3-yl)methyl)triaza-1,2-dien-2-ium-1-ide (gxviii)

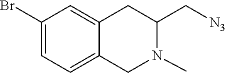 [SMILES: CN1Cc2ccc(Br)cc2CC1CN=[N+]=[N-]]

To a solution of crude gxvii (1.28 g, 3.83 mmol) in anhydrous N,N-dimethylformamide (17 mL) was added sodium azide (373 mg, 5.74 mmol), and the crude mixture was stirred at room temperature for 18 h. After reaction completion, the crude was diluted with dichloromethane, extracted with brine twice, then the organic layer was concentrated under reduced pressure. The crude was purified by flash column chromatography (SiO₂, 50-67% ethyl acetate in hexanes) to give gxviii as a skinny oil (832 mg, 2 steps, 77%). ¹H NMR (600 MHz, d₆-DMSO) δ 7.34 (d, 1H), 7.31 (dd, 1H), 7.04 (d, 1H), 3.73 (d, 1H), 3.55 (d, 1H), 3.50 (dd, 1H), 3.39 (dd, 1H), 2.84-2.78 (m, 2H), 2.67 (dd, 1H), 2.34 (s, 3H).

### (6-Bromo-2-methyl-1,2,3,4-tetrahydroisoquinolin-3-yl)methanamine (gix)

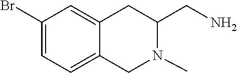

To a solution of gxviii (832 mg, 2.96 mmol) in methanol (30 mL) was added sodium iodide (2.66 g, 17.8 mmol) at room temperature. After 10 minutes, trimethylsilyl chloride (2.27 mL, 17.8 mmol) was added to the reaction mixture, and the reaction mixture was stirred at room temperature for 3 h. After reaction completion, the reaction mixture was quenched with aqueous sodium thiosulfate pentahydrate, then diluted with dichloromethane, extracted with saturated NaHCO 3 (aq), and the organic layer was concentrated under reduced pressure. The crude was purified by flash column chromatography (SiO₂, 10% methanol with 1% triethylamine in dichloromethane) to give gix as a yellow oil (466 mg, 61%). ¹H NMR (400 MHz, d₆-DMSO) δ 7.32-7.25 (m, 2H), 7.01 (dd, 1H), 3.70 (d, 1H), 3.46 (d, 1H), 2.76-2.67 (m, 3H), 2.55 (dd, 1H), 2.49-2.44 (m, 1H), 2.29 (s, 3H), 1.46 (br s, 2H).

### Methyl (2S)-2-amino-3-(3-bromophenyl)propanoate hydrochloride (1:1) (hii)

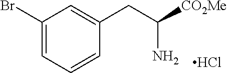 [SMILES: COC(=O)[C@@H](N)Cc1cccc(Br)c1 ; Cl]

The compound was prepared by general procedure a with (2S)-2-amino-3-(3-bromophenyl)-propanoic acid hi (5 g, 20.5 mmol) and thionyl chloride (1.64 mL, 22.5 mmol) in anhydrous methanol (75 mL), refluxed for 18 h. The desired product hii hydrochloride (1:1) was obtained as a white solid (6.03 g, quantitative yield). ¹H NMR (600 MHz, d₆-DMSO) δ 8.39 (br s, 3H), 7.51-7.48 (m, 2H), 7.33-7.28 (m, 1H), 7.25 (d, 1H), 4.32 (t, 1H), 3.69 (s, 3H), 3.11 (d, 2H).

### Methyl (2S)-3-(3-bromophenyl)-2-((ethoxycarbonyl)amino)propanoate (hiii)

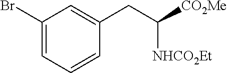

The compound was prepared by general procedure b2 with hii hydrochloride (1:1) (6.03 g, 20.5 mmol), ethyl chloroformate (2.06 mL, 21.5 mmol) and pyridine (3.63 mL, 45.1 mmol) in dichloromethane (90 mL), stirred at 0° C. for 1 h. The desired product hiii was obtained as a yellow oil (6.78 g, quantitative yield). ¹H NMR (600 MHz, d₆-DMSO) δ 7.64 (d, 1H), 7.47 (s, 1H), 7.28-7.24 (m, 2H), 4.24 (ddd, 1H), 3.97-3.89 (m, 2H), 3.63 (s, 3H), 3.04 (dd, 1H), 2.84 (dd, 1H), 1.11 (t, 3H).

### 2-Ethyl 3-methyl (3S)-6-bromo-3,4-dihydroisoquinoline-2,3(1H)-dicarboxylate (hiv)

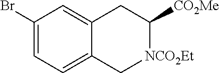

The compound was prepared by general procedure c with hiii (6.78 g, 20.5 mmol) and paraformaldehyde (678 mg, 22.5 mmol) in a mixture of acetic acid (51 mL) and sulfuric acid (17 mL), stirred at room temperature for 18 h. After workup, the crude was purified by flash column chromatography (SiO₂, 20% ethyl acetate in n-hexane) to give a mixture of hiv and 2-ethyl 3-methyl (3S)-8-bromo-3,4-dihydroisoquinoline-2,3(1H)-dicarboxylate as a yellow oil (5.99 g, 85%). The ¹H NMR spectrum of hiv is listed as follows. ¹H NMR (600 MHz, d₆-DMSO) δ 7.53-7.45 (m, 1H), 7.41-7.15 (m, 2H), 5.09-4.87 (m, 1H), 4.68-4.53 (m, 1H), 4.47-4.32 (m, 1H), 4.21-4.03 (m, 2H), 3.60-3.53 (m, 3H), 3.26-3.09 (m, 2H), 1.29-1.21 (m, 1.5H), 1.21-1.12 (m, 1.5H).

### (3S)-6-Bromo-1,2,3,4-tetrahydroisoquinoline-3-carboxylic acid hydrochloride (1:1) (hv)

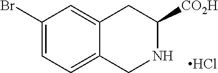 [SMILES: O=C(O)[C@@H]1Cc2cc(Br)ccc2CN1 ; Cl]

The compound was prepared by general procedure d with hiv (5.99 g, 17.5 mmol) in aqueous 8 N hydrogen chloride (175 mL), refluxed for 48 h. A mixture of hv hydrochloride (1:1) and (3S)-8-bromo-1,2,3,4-tetrahydroisoquinoline-3-carboxylic acid was obtained as a white solid (2.5 g, 49%). The ¹H NMR spectrum of hv is listed as follows. ¹H NMR (600 MHz, d₆-DMSO) δ 14.14 (br s, 1H), 9.90 (br s, 1H), 7.54 (d, 1H), 7.47 (dd, 1H), 7.23 (d, 1H), 4.39 (dd, 1H), 4.32 (d, 1H), 4.24 (d, 1H), 3.36-3.31 (m overlapped with H₂O, 1H), 3.12 (dd, 1H).

### Methyl (3S)-6-bromo-1,2,3,4-tetrahydroisoquinoline-3-carboxylate (hvi)

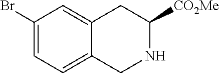 [SMILES: COC(=O)[C@@H]1Cc2cc(Br)ccc2CN1]

The compound was prepared by general procedure e2 with hv hydrochloride (1:1) (2.5 g, 8.54 mmol), thionyl chloride (2.17 mL, 29.9 mmol) and triethylamine (1.19 mL, 8.54 mmol) in anhydrous methanol (43 mL), refluxed for 18 h. The crude was purified by flash column chromatography (SiO₂, 0.5-1% methanol in dichloromethane) to give hvi as a colorless oil (1.24 g, 54%). ¹H NMR (600 MHz, d₆-DMSO) δ 7.34 (d, 1H), 7.29 (dd, 1H), 7.01 (d, 1H), 3.90 (d, 1H), 3.82 (d, 1H), 3.69-3.64 (m, 4H), 2.95 (dd, 1H), 2.85-2.75 (m, 2H).

### Methyl (3S)-6-bromo-2-methyl-1,2,3,4-tetrahydroisoquinoline-3-carboxylate (hxv)

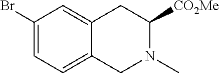 [SMILES: COC(=O)[C@@H]1Cc2cc(Br)ccc2CN1C]

The compound was prepared by general procedure g1 with hvi (1.24 g, 4.59 mmol), 37% aqueous formaldehyde (0.56 mL, 6.89 mmol), sodium triacetoxyborohydride (1.95 g, 9.18 mmol) and acetic acid (0.26 mL, 4.59 mmol) in tetrahydrofuran (23 mL), stirred at room temperature for 2 h. After workup, the crude was purified by flash column chromatography (SiO₂, 2% methanol in dichloromethane) to give the desired product hxv as a white solid (1.29 g, 99%). ¹H NMR (600 MHz, d₆-DMSO) δ 7.35 (s, 1H), 7.30 (dd, 1H), 7.02 (d, 1H), 3.79 (d, 1H), 3.67-3.61 (m, 2H), 3.60 (s, 3H), 3.06 (dd, 1H), 2.96 (dd, 1H), 2.44 (s, 3H).

### ((3S)-6-Bromo-2-methyl-1,2,3,4-tetrahydroisoquinolin-3-yl) methanol (hxvi)

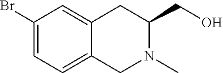

To a solution of hxv (1.29 mg, 4.53 mmol) in anhydrous tetrahydrofuran (23 mL) was added 1 M lithium triethylborohydride in THF (36.2 mL, 36.2 mmol), and the reaction mixture was stirred at room temperature for 18 h. After reaction completion, the crude mixture was quenched with saturated NH₄Cl_{(aq)}, then diluted with water, extracted with dichloromethane, then the organic layer was concentrated under reduced pressure. The crude was purified by flash column chromatography (SiO₂, 6% methanol in dichloromethane) to hxvi as a beige solid (919 mg, 98%). ¹H NMR (600 MHz, d₆-DMSO) δ 7.32 (s, 1H), 7.28 (dd, 1H), 7.02 (d, 1H), 4.57 (t, 1H), 3.71 (d, 1H), 3.58 (dt, 1H), 3.43 (d, 1H), 3.38 (dt, 1H), 2.78 (dd, 1H), 2.70 (dd, 1H), 2.57-2.52 (m, 1H), 2.35 (s, 3H).

### ((3S)-6-Bromo-2-methyl-1,2,3,4-tetrahydroisoquinolin-3-yl)methyl methanesulfonate (hxvii)

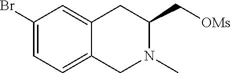 [SMILES: CN1Cc2ccc(Br)cc2C[C@H]1COS(C)(=O)=O]

To a solution of hxvi (919 mg, 3.59 mmol) in anhydrous dichloromethane (36 mL) at 0° C. was added methanesulfonyl chloride (0.36 mL, 4.66 mmol) and triethylamine (0.650 mL, 4.66 mmol), and the reaction mixture was stirred at room temperature for 2 h. After reaction completion, the crude mixture was diluted with dichloromethane, extracted with saturated NaHCO_{3(aq)}, and the organic layer was concentrated under reduced pressure. The crude was used for the next step without further purification.

### 3-(((3S)-6-Bromo-2-methyl-1,2,3,4-tetrahydroisoquinolin-3-yl)methyl)triaza-1,2-dien-2-ium-1-ide (hxviii)

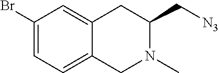 [SMILES: CN1Cc2ccc(Br)cc2C[C@H]1CN=[N+]=[N-]]

To a solution of crude hxvii (952 mg, 2.85 mmol) in anhydrous N,N-dimethylformamide (28 mL) was added sodium azide (278 mg, 4.27 mmol), and the crude mixture was stirred at room temperature for 18 h. After reaction completion, the crude was diluted with dichloromethane, extracted with brine twice, then the organic layer was concentrated under reduced pressure. The crude was purified by flash column chromatography (SiO₂, 50-67% ethyl acetate in hexanes) to give hxviii as a yellow oil (772 mg, 2 steps, 96%). ¹H NMR (600 MHz, d₆-DMSO) δ 7.34 (d, 1H), 7.31 (dd, 1H), 7.04 (d, 1H), 3.73 (d, 1H), 3.55 (d, 1H), 3.50 (dd, 1H), 3.39 (dd, 1H), 2.84-2.78 (m, 2H), 2.67 (dd, 1H), 2.34 (s, 3H).

### ((3S)-6-Bromo-2-methyl-1,2,3,4-tetrahydroisoquinolin-3-yl)methanamine (hix)

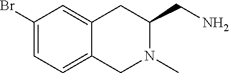

To a solution of hxviii (381 mg, 1.36 mmol) in methanol (13 mL) was added sodium iodide (1.22 g, 8.13 mmol) at room temperature. After 10 minutes, trimethylsilyl chloride (1.04 mL, 8.13 mmol) was added to the reaction mixture, and the reaction mixture was stirred at room temperature for 2 h. After reaction completion, the reaction mixture was quenched with aqueous sodium thiosulfate pentahydrate, then diluted with dichloromethane, extracted with saturated NaHCO_{3(aq)}, and the organic layer was concentrated under reduced pressure. The crude was purified by flash column chromatography (SiO₂, 10% methanol with 1% triethylamine in dichloromethane) to give hix as a yellow oil (208 mg, 60%). ¹H NMR (600 MHz, d₆-DMSO) δ 7.33-7.25 (m, 2H), 7.02 (d, 1H), 3.71 (d, 1H), 3.50 (d, 1H), 2.83 (br s, 2H), 2.75-2.71 (m, 3H), 2.62-2.53 (m, 2H), 2.28 (s, 3H).

### Methyl (2R)-2-amino-3-(4-methylphenyl)propanoate hydrochloride (iii)

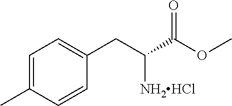

The compound was prepared by general procedure a with (2R)-2-amino-3-(4-ethylphenyl) propanoic acid ii (5.1 g, 28.5 mmol) and thionyl chloride (4.13 mL, 56.9 mmol) in anhydrous methanol (130 mL), stirred at 50° C. for 17 h. The desired product iii was obtained as a white solid (6.50 g, 99%). ¹H NMR (600 MHz, DMSO-d₆) δ 8.52 (br s, 3H), 7.15 (d, 2H), 7.11 (d, 2H), 4.24 (t, 1H), 3.67 (s, 3H), 3.11 (dd, 1H), 3.04 (dd, 1H) 2.28 (s, 3H).

### Methyl (2R)-2-((ethoxycarbonyl)amino)-3-(4-methylphenyl)propanoate (iiii)

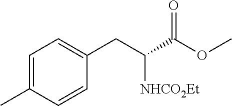

The compound was prepared by general procedure b2 with iii (6.5 g, 28.3 mmol), ethyl chloroformate (4.17 mL, 29.7 mmol) and pyridine (5.01 mL, 62.3 mmol) in dichloromethane (130 mL), stirred at 0° C. for 2 h. After workup, the crude was purified by Isco Combi-Flash Companion column chromatography (0-20% ethyl acetate in n-hexane) to give iiii (7.5 g, 99%). ¹H NMR (600 MHz, DMSO-d₆) δ 7.59 (d, 1H), 7.12 (d, 2H), 7.09 (d, 2H), 4.18 (ddd, 1H), 4.11 (q, 0.1H), 3.98-3.86 (m, 1.9H), 3.62 (s, 3H), 2.97 (dd, 1H), 2.81 (dd, 1H), 2.26 (s, 3H), 1.21 (t, 0.2H), 1.12 (t, 2.6H).

### 2-Ethyl 3-methyl (3R)-7-methyl-3,4-dihydroisoquinoline-2,3(1H)-dicarboxylate (iiv)

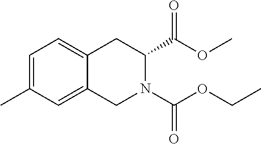 [SMILES: CCOC(=O)N1Cc2cc(C)ccc2C[C@@H]1C(=O)OC]

The compound was prepared by general procedure c with iiii (7.5 g, 28.3 mmol) and paraformaldehyde (0.93 g, 31.1 mmol) in a mixture of acetic acid (70 mL) and sulfuric acid (30 mL), stirred at room temperature for 17 h. After workup, the crude was purified by Isco Combi-Flash Companion column chromatography (0-30% ethyl acetate in n-hexane) to give iiv as a colorless liquid (6 g, 77%). ¹H NMR (400 MHz, DMSO-d₆) δ 7.08 (dd, 1H), 7.04-6.97 (m, 2H), 4.96 (t, 0.5H), 4.86 (t, 0.5H), 4.59 (dd, 1H), 4.40 (dd, 1H), 4.19-4.06 (m, 2H), 3.55 (s, 1.5H), 3.53 (s, 1.5H), 3.18-3.02 (m, 2H), 2.25 (s, 3H), 1.25 (t, 1.5H), 1.16 (t, 1.5H).

### (3R)-7-Methyl-1,2,3,4-tetrahydroisoquinoline-3-carboxylic acid hydrochloride (1:1) (iv)

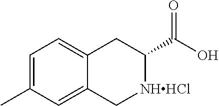

The compound was prepared by general procedure d with 2 iiv (6 g, 21.6 mmol) in aqueous 8 N hydrogen chloride (100 mL), refluxed for 17 h. The desired product iv hydrochloride (1:1) was obtained as a white solid (4.13 g, 84%). ¹H NMR (400 MHz, DMSO-d₆) δ 14.09 (br s, 1H), 9.85 (br s, 2H), 7.15 (d, 1H), 7.12-7.03 (m, 2H), 4.37 (dd, 1H), 4.27 (s, 2H), 3.25 (dd, 1H), 3.07 (dd, 1H), 2.27 (s, 3H).

### Methyl (3R)-7-methyl-1,2,3,4-tetrahydroisoquinoline-3-carboxylate (ivi)

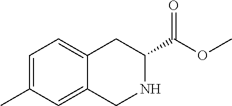

The compound was prepared by general procedure e2 with iv hydrochloride (1:1) (4.13 g, 18.1 mmol) and thionyl chloride (4.61 mL, 63.5 mmol) in anhydrous methanol (70 mL), stirred at 50° C. for 17 h. After workup, the crude was concentrated to give the desired product ivi (3.7 g, 99%). ¹H NMR (400 MHz, DMSO-d₆) δ 6.98 (d, 1H), 6.91 (d, 1H), 6.83 (s, 1H), 3.91 (d, 1H), 3.83 (d, 1H), 3.70-3.61 (m, 4H), 2.89 (dd, 1H), 2.76 (dd, 1H), 2.70 (br s, 1H), 2.22 (s, 3H).

### (3R)-7-Methyl-1,2,3,4-tetrahydroisoquinoline-3-carboxamide (ivii)

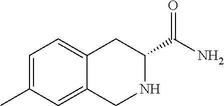

The compound was prepared by general procedure f with ivi (3.7 g, 18 mmol) in 28-30% aqueous ammonium hydroxide (120 mL), stirred at room temperature for 17 h, to give ivii (2.92 g, 85%). ¹H NMR (400 MHz, DMSO-d₆) δ 7.31 (br s, 1H), 7.05 (br s, 1H), 6.97 (d, 1H), 6.91 (d, 1H), 6.83 (s, 1H), 3.88 (d, 1H), 3.82 (d, 1H), 3.35-3.28 (m overlapped with H₂O, 1H), 2.83 (dd, 1H), 2.65 (dd, 1H), 2.22 (s, 3H).

### (3R)-2,7-Dimethyl-1,2,3,4-tetrahydroisoquinoline-3-carboxamide (iviii)

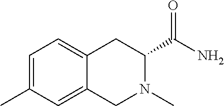

The compound was prepared by general procedure g1 with ivii (2.92 g, 15.3 mmol), 37% aqueous formaldehyde (1.71 mL, 23 mmol), sodium triacetoxyborohydride (4.66 g, 22 mmol) and acetic acid (0.88 mL, 15.3 mmol) in acetonitrile (60 mL) and tetrahydrofuran (60 mL), stirred at room temperature for 2 h. After workup, the crude was purified by Isco Combi-Flash Companion column chromatography (0-5% methanol in CH₂Cl₂) to give iviii (1.9 g, 61%). ¹H NMR (600 MHz, DMSO-d₆) δ 7.36 (br s, 1H), 7.04 (br s, 1H), 6.98 (d, 1H), 6.93 (d, 1H), 6.86 (s, 1H), 3.83 (d, 1H), 3.44 (d, 1H), 3.01 (dd, 1H), 2.88 (dd, 1H), 2.78 (dd, 1H), 2.30 (s, 3H), 2.23 (s, 3H).

### ((3R)-2,7-Dimethyl-1,2,3,4-tetrahydroisoquinolin-3-yl)methanamine (iix)

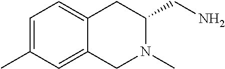

The compound was prepared by general procedure h with iviii (0.2 g, 0.979 mmol) and 2M lithium aluminium hydride in THF (1.47 mL, 2.94 mmol) in anhydrous tetrahydrofuran (10 mL), stirred at 60° C. for 17 h. After workup, the crude was purified by Isco Combi-Flash Companion column chromatography (0-5% methanol with 1% NH₄OH in CH₂Cl₂) to give iix (0.146 g, 78%). ¹H NMR (400 MHz, DMSO-d₆) δ 6.96 (d, 1H), 6.92 (d, 1H), 6.84 (s, 1H), 3.69 (d, 1H), 3.49 (d, 1H), 2.72 (dd, 1H), 2.67 (d, 2H), 2.56 (dd, 1H), 2.49-2.43 (m, 1H), 2.29 (s, 3H), 2.23 (s, 3H).

### Methyl (2S)-2-amino-3-(4-methylphenyl)propanoate hydrochloride (jii)

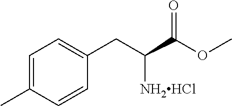

The compound was prepared by general procedure a with (2S)-2-amino-3-(4-ethylphenyl) propanoic acid ji (5 g, 27.9 mmol) and thionyl chloride (4.05 mL, 55.8 mmol) in anhydrous methanol (130 mL), stirred at 50° C. for 17 h. The desired product jii was obtained as a white solid (6.4 g, 99%). ¹H NMR (600 MHz, DMSO-d₆) δ 8.68 (br s, 3H), 7.15 (d, 2H), 7.12 (d, 2H), 4.21 (dd, 1H), 3.67 (s, 3H), 3.15 (dd, 1H), 3.07 (dd, 1H), 2.29 (s, 3H).

### Methyl (2S)-2-((ethoxycarbonyl)amino)-3-(4-methylphenyl)propanoate (jiii)

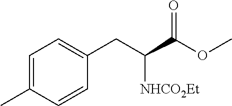

The compound was prepared by general procedure b2 with jii (6.4 g, 27.9 mmol), ethyl chloroformate (4.11 mL, 29.3 mmol) and pyridine (4.94 mL, 61.3 mmol) in dichloromethane (130 mL), stirred at 0° C. for 2 h. After workup, the crude was purified by Isco Combi-Flash Companion column chromatography (0-20% ethyl acetate in n-hexane) to give jiii (7.3 g, 99%). ¹H NMR (400 MHz, DMSO-d₆) δ 7.59 (d, 1H), 7.12 (d, 2H), 7.09 (d, 2H), 4.19 (ddd, 1H), 3.99-3.86 (m, 2H), 3.62 (s, 3H), 2.97 (dd, 1H), 2.81 (dd, 1H), 2.26 (s, 3H), 1.12 (t, 3H).

### 2-Ethyl 3-methyl (3S)-7-methyl-3,4-dihydroisoquinoline-2,3(1H)-dicarboxylate (jiv)

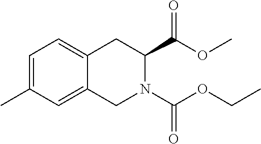 [SMILES: CCOC(=O)N1Cc2cc(C)ccc2C[C@H]1C(=O)OC]

The compound was prepared by general procedure c with jiii (7.3 g, 27.5 mmol) and paraformaldehyde (0.931 g, 30.3 mmol) in a mixture of acetic acid (70 mL) and sulfuric acid (30 mL), stirred at room temperature for 17 h. After workup, the crude was purified by Isco Combi-Flash Companion column chromatography (0-30% ethyl acetate in n-hexane) to give jiv as a colorless liquid (5.50 g, 72%). ¹H NMR (600 MHz, DMSO-d₆) δ 7.11-6.96 (m, 3H), 4.95 (t, 0.5H), 4.86 (t, 0.5H) 4.59 (dd, 1H), 4.40 (dd, 1H), 4.17-4.00 (m, 2H), 3.55 (s, 1.5H), 3.53 (s, 1.5H), 3.20-3.01 (m, 2H), 2.25 (s, 3H), 1.25 (t, 1.5H), 1.16 (t, 1.5H).

### (3S)-7-Methyl-1,2,3,4-tetrahydroisoquinoline-3-carboxylic acid hydrochloride (1:1) (jv)

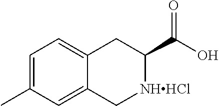

The compound was prepared by general procedure d with jiv (6 g, 21.6 mmol) in aqueous 8 N hydrogen chloride (100 mL), refluxed for 17 h. The desired product jv hydrochloride (1:1) was obtained as a white solid (4.13 g, 84%). ¹H NMR (600 MHz, DMSO-d₆) δ 14.10 (br s, 1H), 9.86 (br s, 2H), 7.15 (d, 1H), 7.08 (d, 1H), 7.05 (s, 1H), 4.37 (dd, 1H), 4.27 (s, 2H), 3.25 (dd, 1H), 3.07 (dd, 1H), 2.27 (s, 3H).

### Methyl (3S)-7-methyl-1,2,3,4-tetrahydroisoquinoline-3-carboxylate (jvi)

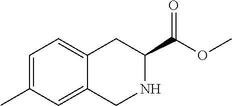

The compound was prepared by general procedure e2 with jv hydrochloride (1:1) (4.06 g, 17.8 mmol) and thionyl chloride (4.53 mL, 62.4 mmol) in anhydrous methanol (70 mL), stirred at 50° C. for 17 h. After workup, the crude was concentrated to give jvi (3.6 g, 98%). ¹H NMR (400 MHz, DMSO-d₆) δ 6.98 (d, 1H), 6.91 (d, 1H), 6.83 (s, 1H), 3.91 (d, 1H), 3.83 (d, 1H), 3.66 (s, 3H), 3.65-3.62 (m, 1H), 2.89 (dd, 1H), 2.76 (dd, 1H), 2.70 (br s, 1H), 2.22 (s, 3H).

### (3S)-7-Methyl-1,2,3,4-tetrahydroisoquinoline-3-carboxamide (ivii)

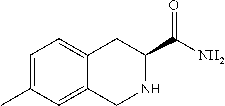

The compound was prepared by general procedure f with jvi (3.6 g, 17.5 mmol) in 28-30% aqueous ammonium hydroxide (120 mL), stirred at room temperature for 17 h, to give jvii (2.74 g, 82%). ¹H NMR (400 MHz, DMSO-d₆) δ 7.31 (br s, 1H), 7.05 (br s, 1H), 6.97 (d, 1H), 6.91 (d, 1H), 6.83 (s, 1H), 3.88 (d, 1H), 3.82 (d, 1H), 3.35-3.27 (m overlapped with H₂O, 1H), 2.83 (dd, 1H), 2.65 (dd, 1H), 2.22 (s, 3H).

### (3S)-2,7-Dimethyl-1,2,3,4-tetrahydroisoquinoline-3-carboxamide (jviii)

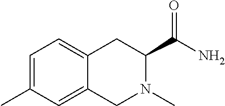

The compound was prepared by general procedure g1 with jvii (2.74 g, 14.4 mmol), 37% aqueous formaldehyde (1.61 mL, 21.6 mmol), sodium triacetoxyborohydride (6.1 g, 28.8 mmol) and acetic acid (0.820 mL, 14.4 mmol) in acetonitrile (60 mL) and tetrahydrofuran (60 mL), stirred at room temperature for 2 h. After workup, the crude was purified by Isco Combi-Flash Companion column chromatography (0-5% methanol in CH₂Cl₂) to give jviii (0.74 g, 25%). ¹H NMR (400 MHz, DMSO-d₆) δ 7.37 (br s, 1H), 7.04 (br s, 1H), 6.98 (d, 1H), 6.92 (d, 1H), 6.85 (s, 1H), 3.83 (d, 1H), 3.44 (d, 1H), 3.01 (dd, 1H), 2.88 (dd, 1H), 2.78 (dd, 1H), 2.30 (s, 3H), 2.23 (s, 3H).

### ((3S)-2,7-Dimethyl-1,2,3,4-tetrahydroisoquinolin-3-yl)methanamine (jix)

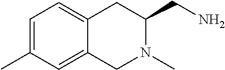

The compound was prepared by general procedure h with jviii (0.2 g, 0.979 mmol) and 2M lithium aluminium hydride in THF (1.47 mL, 2.94 mmol) in anhydrous tetrahydrofuran (10 mL), stirred at 60° C. for 17 h. After workup, the crude was purified by Isco Combi-Flash Companion column chromatography (0-5% methanol with 1% ammonia hydroxide in CH₂Cl₂) to give jix (0.146 g, 78%). ¹H NMR (400 MHz, DMSO-d₆) δ 6.96 (d, 1H), 6.92 (d, 1H), 6.83 (s, 1H), 3.69 (d, 1H), 3.49 (d, 1H), 2.72 (dd, 1H), 2.67 (d, 2H), 2.56 (dd, 1H), 2.50-2.43 (m, 1H), 2.29 (s, 3H), 2.23 (s, 3H), 1.52 (br s, 2H).

### Methyl (2R)-2-amino-3-(4-fluorophenyl)propanoate hydrochloride (kii)

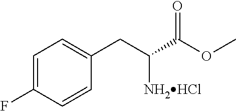

The compound was prepared by general procedure a with (2R)-2-amino-3-(4-fluorophenyl)-propanoic acid ki (5 g, 27.3 mmol) and thionyl chloride (3.96 mL, 54.6 mmol) in anhydrous methanol (130 mL), stirred at 50° C. for 17 h. The desired product kii was obtained as a white solid (6.21 g, 97%). ¹H NMR (400 MHz, DMSO-d₆) δ 8.57 (br s, 3H), 7.33-7.23 (m, 2H), 7.22-7.11 (m, 2H), 4.28 (dd, 1H), 3.68 (s, 3H), 3.22-3.06 (m, 2H).

### Methyl (2R)-2-((ethoxycarbonyl)amino)-3-(4-fluorophenyl)propanoate (kiii)

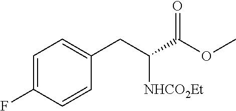

The compound was prepared by general procedure b2 with kii (6.21 g, 26.6 mmol), ethyl chloroformate (3.92 mL, 27.9 mmol) and pyridine (4.71 mL, 58.5 mmol) in dichloromethane (130 mL), stirred at 0° C. for 2 h. After workup, the crude was purified by Isco Combi-Flash Companion column chromatography (0-20% ethyl acetate in n-hexane) to give kiii (7.1 g, 99%). ¹H NMR (600 MHz, DMSO-d₆) δ 7.62 (d, 1H), 7.27 (dd, 2H), 7.10 (dd, 2H), 4.20 (ddd, 1H), 4.10 (q, 0.2H), 3.99-3.86 (m, 1.8H), 3.61 (s, 3H), 3.01 (dd, 1H), 2.84 (dd, 1H), 1.20 (t, 0.3H), 1.11 (t, 2.7H).

### 2-Ethyl 3-methyl (3R)-7-fluoro-3,4-dihydroisoquinoline-2,3(1H)-dicarboxylate (kiv)

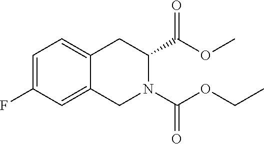 [SMILES: CCOC(=O)N1Cc2cc(F)ccc2C[C@@H]1C(=O)OC]

The compound was prepared by general procedure c with kiii (7.1 g, 26.4 mmol) and paraformaldehyde (0.87 g, 29 mmol) in a mixture of acetic acid (70 mL) and sulfuric acid (30 mL), stirred at room temperature for 17 h. After workup, the crude was purified by Isco Combi-Flash Companion column chromatography (0-30% ethyl acetate in n-hexane) to give kiv as a colorless liquid (6.88 g, 93%). ¹H NMR (600 MHz, DMSO-d₆) δ 7.25 (dd, 1H), 7.14 (dd, 1H), 7.02 (t, 1H), 4.99 (t, 0.5H), 4.90 (t, 0.5H), 4.64 (dd, 1H), 4.47 (dd, 1H), 4.14 (q, 1H), 4.13-4.03 (m, 1H), 3.57 (s, 1.5H), 3.55 (s, 1.5H), 3.21-3.07 (m, 2H), 1.25 (t, 1.5H), 1.17 (t, 1.5H).

### (3R)-7-Fluoro-1,2,3,4-tetrahydroisoquinoline-3-carboxylic acid hydrochloride (1:1) (kv)

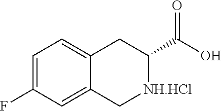

The compound was prepared by general procedure d with kiv (6.88 g, 24.5 mmol) in aqueous 8 N hydrogen chloride (100 mL), refluxed for 17 h. The desired product kv hydrochloride (1:1) was obtained as a white solid (3.66 g, 77%). ¹H NMR (400 MHz, DMSO-d₆) δ 9.99 (br s, 2H), 7.33 (dd, 1H), 7.24-7.08 (m, 2H), 4.40 (dd, 1H), 4.36-4.25 (m, 2H), 3.31 (dd, 1H), 3.09 (dd, 1H).

### Methyl (3R)-7-Fluoro-1,2,3,4-tetrahydroisoquinoline-3-carboxylate (kvi)

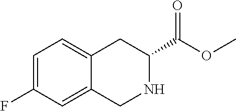

The compound was prepared by general procedure e2 with kv hydrochloride (1:1) (3.66 g, 18.8 mmol) and thionyl chloride (4.76 mL, 65.6 mmol) in anhydrous methanol (70 mL), stirred at 50° C. for 17 h. After workup, the crude was concentrated to give kvi (3.25 g, 83%). ¹H NMR (400 MHz, DMSO-d₆) δ 7.14 (dd, 1H), 7.00-6.86 (m, 2H), 3.95 (d, 1H), 3.86 (d, 1H), 3.71-3.62 (m, 4H), 2.93 (dd, 1H), 2.82-2.69 (m, 2H).

### (3R)-7-Fluoro-1,2,3,4-tetrahydroisoquinoline-3-carboxamide (kvii)

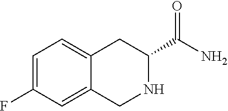 [SMILES: NC(=O)[C@H]1Cc2ccc(F)cc2CN1]

The compound was prepared by general procedure f with kvi (3.25 g, 15.5 mmol) in 28-30% aqueous ammonium hydroxide (120 mL), stirred at room temperature for 17 h, to give kvii (2.07 g, 69%). ¹H NMR (400 MHz, DMSO-d₆) δ 7.32 (br s, 1H), 7.13 (dd, 1H), 7.07 (br s, 1H), 6.98-6.83 (m, 2H), 3.92 (d, 1H), 3.84 (d, 1H), 3.38-3.28 (m overlapped with H₂O, 1H), 2.87 (dd, 1H), 2.66 (dd, 1H), 2.56 (br s, 1H).

### (3R)-7-Fluoro-2-methyl-1,2,3,4-tetrahydroisoquinoline-3-carboxamide (kviii)

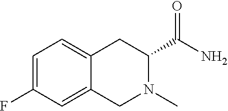

The compound was prepared by general procedure g1 with kvii (2.07 g, 10.7 mmol), 37% aqueous formaldehyde (1.17 mL, 15 mmol), sodium triacetoxyborohydride (4.52 g, 21.3 mmol) and acetic acid (0.61 mL, 10.7 mmol) in acetonitrile (60 mL) and tetrahydrofuran (60 mL), stirred at room temperature for 2 h. After workup, the crude was purified by Isco Combi-Flash Companion column chromatography (0-5% methanol in CH₂Cl₂) to give kviii (2.12 g, 96%). ¹H NMR (400 MHz, DMSO-d₆) δ 7.39 (br s, 1H), 7.14 (dd, 1H), 7.07 (br s, 1H), 6.99-6.87 (m, 2H), 3.88 (d, 1H), 3.50 (d, 1H), 3.08 (dd, 1H), 2.90 (dd, 1H), 2.83 (dd, 1H), 2.31 (s, 3H).

### ((3R)-7-Fluoro-2-methyl-1,2,3,4-tetrahydroisoquinolin-3-yl)methanamine (kix)

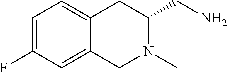

The compound was prepared by general procedure h with kviii (0.2 g, 0.96 mmol) and 2M lithium aluminium hydride in THF (1.44 mL, 2.88 mmol) in anhydrous tetrahydrofuran (10 mL), stirred at 60° C. for 17 h. After workup, the crude was purified by Isco Combi-Flash Companion column chromatography (0-5% methanol with 1% ammonia hydroxide in CH₂Cl₂) to give kix (0.14 g, 75%). ¹H NMR (400 MHz, DMSO-d₆) δ 7.16-6.86 (m, 3H), 3.74 (d, 1H), 3.52 (d, 1H), 2.76-2.67 (m, 3H), 2.56 (dd, 1H), 2.51-2.46 (m, 1H), 2.29 (s, 3H).

### Methyl (2S)-2-amino-3-(4-fluorophenyl)propanoate hydrochloride (lii)

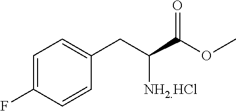

The compound was prepared by general procedure a with (2S)-2-amino-3-(4-fluorophenyl)-propanoic acid li (5 g, 27.3 mmol) and thionyl chloride (3.96 mL, 54.6 mmol) in anhydrous methanol (130 mL), stirred at 50° C. for 17 h. The desired product lii was obtained as a white solid (6.37 g, 99%). ¹H NMR (400 MHz, DMSO-d₆) δ 8.75-8.50 (br, 3H), 7.37-7.23 (m, 2H), 7.24-7.07 (m, 2H), 4.28 (t, 1H), 3.68 (s, 3H), 3.23-3.03 (m, 2H).

### Methyl (2S)-2-((ethoxycarbonyl)amino)-3-(4-fluorophenyl)propanoate (liii)

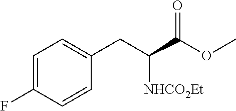

The compound was prepared by general procedure b2 with lii (6.37 g, 27.3 mmol), ethyl chloroformate (4.02 mL, 28.6 mmol) and pyridine (4.83 mL, 60 mmol) in dichloromethane (130 mL), stirred at 0° C. for 2 h. After workup, the crude was purified by Isco Combi-Flash Companion column chromatography (0-20% ethyl acetate in n-hexane) to give liii (7.34 g, 99%). ¹H NMR (400 MHz, DMSO-d₆) δ 7.62 (d, 1H), 7.28 (dd, 2H), 7.10 (dd, 2H), 4.21 (ddd, 1H), 4.10 (q, 0.3H), 4.00-3.84 (m, 1.7H), 3.61 (s, 3H), 3.01 (dd, 1H), 2.84 (dd, 1H), 1.20 (0.5H), 1.11 (t, 2.5H).

### 2-Ethyl 3-methyl (3S)-7-fluoro-3,4-dihydroisoquinoline-2,3(1H)-dicarboxylate (liv)

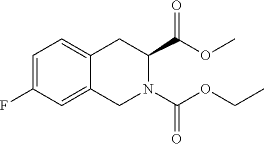 [SMILES: CCOC(=O)N1Cc2cc(F)ccc2C[C@H]1C(=O)OC]

The compound was prepared by general procedure c with liii (7.34 g, 27.3 mmol) and paraformaldehyde (0.9 g, 30 mmol) in a mixture of acetic acid (70 mL) and sulfuric acid (30 mL), stirred at room temperature for 17 h. After workup, the crude was purified by Isco Combi-Flash Companion column chromatography (0-30% ethyl acetate in n-hexane) to give liv as a colorless liquid (6.49 g, 85%). ¹H NMR (400 MHz, DMSO-d₆) δ 7.30-7.22 (m, 1H), 7.20-7.10 (m, 1H), 7.09-6.96 (m, 1H), 4.99 (t, 0.5H), 4.89 (t, 0.5H), 4.65 (dd, 1H), 4.46 (dd, 1H), 4.25-4.00 (m, 2H), 3.57 (s, 1.5H), 3.55 (s, 1.5H), 3.22-3.06 (m, 2H), 1.25 (t, 1.5H), 1.16 (t, 1.5H).

### (3S)-7-Fluoro-1,2,3,4-tetrahydroisoquinoline-3-carboxylic acid hydrochloride (1:1) (lv)

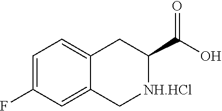

The compound was prepared by general procedure d with liv (6.49 g, 23.1 mmol) in aqueous 8 N hydrogen chloride (100 mL), refluxed for 17 h. The desired product lv hydrochloride (1:1) was obtained as a white solid (3.66 g, 77%). ¹H NMR (400 MHz, DMSO-d₆) δ 9.98 (br s, 2H), 7.33 (dd, 1H), 7.22-7.09 (m, 2H), 4.40 (dd, 1H), 4.40-4.26 (m, 2H), 3.31 (dd, 1H), 3.10 (dd, 1H).

### Methyl (3S)-7-fluoro-1,2,3,4-tetrahydroisoquinoline-3-carboxylate (lvi)

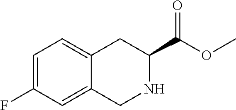

The compound was prepared by general procedure e2 with lv hydrochloride (1:1) (3.22 g, 16.5 mmol) and thionyl chloride (4.19 mL, 57.7 mmol) in anhydrous methanol (100 mL), stirred at 50° C. for 17 h. After workup, the crude was concentrated to give Ivi (2.88 g, 83%). ¹H NMR (400 MHz, DMSO-d₆) δ 7.22-7.10 (m, 1H), 7.02-6.84 (m, 2H), 3.96 (d, 1H), 3.87 (d, 1H), 3.72-3.62 (m, 4H), 2.94 (dd, 1H), 2.83-2.71 (m, 2H).

### (3S)-7-Fluoro-1,2,3,4-tetrahydroisoquinoline-3-carboxamide (lvii)

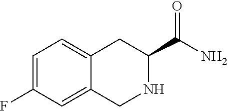 [SMILES: NC(=O)[C@@H]1Cc2ccc(F)cc2CN1]

The compound was prepared by general procedure f with lvi (2.88 g, 13.8 mmol) in 28-30% aqueous ammonium hydroxide (100 mL), stirred at room temperature for 17 h, to give Ivii (1.84 g, 69%). ¹H NMR (400 MHz, DMSO-d₆) δ 7.32 (br s, 1H), 7.13 (dd, 1H), 7.07 (br s, 1H), 6.98-6.83 (m, 2H), 3.92 (d, 1H), 3.84 (d, 1H), 3.36-3.32 (m, 1H), 2.87 (dd, 1H), 2.72-2.55 (m, 2H).

### (3S)-7-Fluoro-2-methyl-1,2,3,4-tetrahydroisoquinoline-3-carboxamide (lviii)

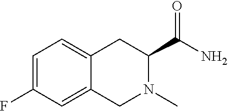

The compound was prepared by general procedure g1 with Ivii (1.84 g, 9.47 mmol), 37% aqueous formaldehyde (1.06 mL, 14.2 mmol), sodium triacetoxyborohydride (4.02 g, 18.9 mmol) and acetic acid (0.54 mL, 9.47 mmol) in acetonitrile (50 mL) and tetrahydrofuran (50 mL), stirred at room temperature for 2 h. After workup, the crude was purified by Isco Combi-Flash Companion column chromatography (0-5% methanol in CH₂Cl₂) to give Iviii (1.75 g, 89%). ¹H NMR (400 MHz, DMSO-d₆) δ 7.39 (br s, 1H), 7.14 (dd, 1H), 7.07 (br s, 1H), 7.00-6.88 (m, 2H), 3.88 (d, 1H), 3.50 (d, 1H), 3.08 (dd, 1H), 2.90 (dd, 1H), 2.83 (dd, 1H), 2.31 (s, 3H).

### ((3S)-7-Fluoro-2-methyl-1,2,3,4-tetrahydroisoquinolin-3-yl)methanamine (lix)

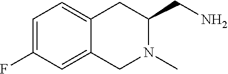

The compound was prepared by general procedure h Iviii (0.2 g, 0.96 mmol) and 2M lithium aluminium hydride in THF (1.44 mL, 2.88 mmol) in anhydrous tetrahydrofuran (10 mL), stirred at 60° C. for 17 h. After workup, the crude was purified by Isco Combi-Flash Companion column chromatography (0-5% methanol with 1% ammonia hydroxide in CH₂Cl₂) to give Iix (0.137 g, 73%). ¹H NMR (400 MHz, DMSO-d₆) δ 7.16-7.06 (m, 1H), 7.01-6.83 (m, 2H), 3.74 (d, 1H), 3.52 (d, 1H), 2.76-2.67 (m, 3H), 2.56 (dd, 1H), 2.50-2.45 (m, 1H), 2.29 (s, 3H).

### Methyl (2R)-2-amino-3-(4-chlorophenyl)propanoate hydrochloride (mii)

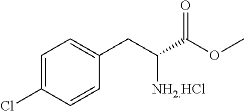

The compound was prepared by general procedure a with (2R)-2-amino-3-(4-chlorophenyl)-propanoic acid mi (5.1 g, 25.5 mmol) and thionyl chloride (3.71 mL, 51.1 mmol) in anhydrous methanol (100 mL), stirred at 50° C. for 17 h. The desired product methyl mii was obtained as a white solid (6.38 g, 99%). ¹H NMR (400 MHz, DMSO-d₆) δ 8.65 (br s, 3H), 7.40 (d, 2H), 7.28 (d, 2H), 4.28 (dd, 1H), 3.68 (s, 3H), 3.17 (dd, 1H), 3.11 (dd, 1H).

### Methyl (2R)-3-(4-chlorophenyl)-2-((ethoxycarbonyl)amino)propanoate (miii)

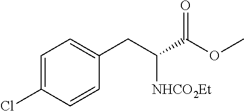

The compound was prepared by general procedure b2 with mii (6.38 g, 25.5 mmol), ethyl chloroformate (3.76 mL, 26.8 mmol) and pyridine (4.52 mL, 56.1 mmol) in dichloromethane (130 mL), stirred at 0° C. for 2 h. After workup, the crude was purified by Isco Combi-Flash Companion column chromatography (0-30% ethyl acetate in n-hexane) to give miii (7.2 g, 99%). ¹H NMR (400 MHz, DMSO-d₆) δ 7.64 (d, 1H), 7.35 (d, 2H), 7.27 (d, 2H), 4.23 (ddd, 1H), 4.11 (q, 0.2H), 4.01-3.85 (m, 1.8H), 3.63 (s, 3H), 3.03 (dd, 1H), 2.85 (dd, 1H), 1.21 (t, 0.3H), 1.11 (t, 2.7H).

### 2-Ethyl 3-methyl (3R)-7-chloro-3,4-dihydroisoquinoline-2,3(1H)-dicarboxylate (miv)

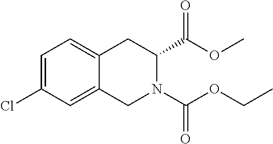 [SMILES: CCOC(=O)N1Cc2cc(Cl)ccc2C[C@@H]1C(=O)OC]

The compound was prepared by general procedure c with miii (7.2 g, 25.2 mmol) and paraformaldehyde (0.83 g, 30 mmol) in a mixture of acetic acid (70 mL) and sulfuric acid (30 mL), stirred at room temperature for 17 h. After workup, the crude was purified by Isco Combi-Flash Companion column chromatography (0-30% ethyl acetate in n-hexane) to give miv as a colorless liquid (5.40 g, 72%). ¹H NMR (400 MHz, DMSO-d₆) δ 7.37 (d, 1H), 7.28-7.18 (m, 2H), 5.00 (t, 0.5H), 4.91 (t, 0.5H), 4.65 (dd, 1H), 4.46 (dd, 1H), 4.21-4.01 (m, 2H), 3.57 (s, 1.5H), 3.56 (s, 1.5H), 3.24-3.06 (m, 2H), 1.25 (t, 1.6H), 1.17 (t, 1.4H).

### (3R)-7-Chloro-1,2,3,4-tetrahydroisoquinoline-3-carboxylic acid hydrochloride (1:1) (mv)

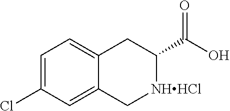

The compound was prepared by general procedure d with miv (5.4 g, 18.1 mmol) in aqueous 8 N hydrogen chloride (100 mL), refluxed for 17 h. The desired product mv hydrochloride (1:1) was obtained as a white solid (3.03 g, 79%). ¹H NMR (400 MHz, DMSO-d₆) δ 9.91 (br s, 2H), 7.41 (s, 1H), 7.35 (dd, 1H), 7.31 (d, 1H), 4.40 (dd, 1H), 4.35 (d, 1H), 4.29 (d, 1H), 3.32 (dd, 1H), 3.09 (dd, 1H).

### Methyl (3R)-7-chloro-1,2,3,4-tetrahydroisoquinoline-3-carboxylate (mvi)

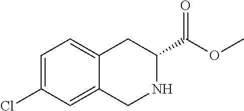

The compound was prepared by general procedure e2 with my hydrochloride (1:1) (3.03 g, 14.3 mmol) and thionyl chloride (3.63 mL, 50.1 mmol) in anhydrous methanol (100 mL), stirred at 50° C. for 17 h. After workup, the crude was concentrated to give the desired product mvi (2.78 g, 86%). ¹H NMR (400 MHz, DMSO-d₆) δ 7.19-7.08 (m, 3H), 3.94 (d, 1H), 3.85 (d, 1H), 3.70-3.64 (m, 4H), 2.93 (dd, 1H), 2.83-2.72 (m, 2H).

### (3R)-7-Chloro-1,2,3,4-tetrahydroisoquinoline-3-carboxamide (mvii)

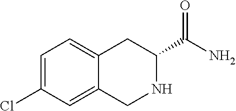 [SMILES: NC(=O)[C@H]1Cc2ccc(Cl)cc2CN1]

The compound was prepared by general procedure f with mvi (2.78 g, 12.3 mmol) in 28-30% aqueous ammonium hydroxide (100 mL), stirred at room temperature for 17 h, to give mvii (2.35 g, 90%). ¹H NMR (400 MHz, DMSO-d₆) δ 7.34 (br s, 1H), 7.19-7.11 (m, 3H), 7.09 (br s, 1H), 3.93 (d, 1H), 3.84 (d, 1H), 3.39-3.27 (m overlapped with H₂O, 1H), 2.88 (dd, 1H), 2.68 (dd, 1H), 2.63 (br s, 1H).

### (3R)-7-Chloro-2-methyl-1,2,3,4-tetrahydroisoquinoline-3-carboxamide (mviii)

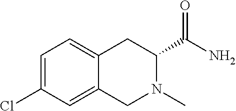

The compound was prepared by general procedure g1 with mvii (2.35 g, 11.1 mmol), 37% aqueous formaldehyde (1.24 mL, 16.7 mmol), sodium triacetoxyborohydride (4.71 g, 22.2 mmol) and acetic acid (0.630 mL, 11.1 mmol) in acetonitrile (50 mL) and tetrahydrofuran (50 mL), stirred at room temperature for 2 h. After workup, the crude was purified by Isco Combi-Flash Companion column chromatography (0-5% methanol in CH₂Cl₂) to give mviii (1.71 g, 69%). ¹H NMR (400 MHz, DMSO-d₆) δ 7.41 (br s, 1H), 7.22-7.12 (m, 3H), 7.08 (br s, 1H), 3.88 (d, 1H), 3.51 (d, 1H), 3.10 (dd, 1H), 2.98-2.79 (m, 2H), 2.32 (s, 3H).

### ((3R)-7-Chloro-2-methyl-1,2,3,4-tetrahydroisoquinolin-3-yl)methanamine (mix)

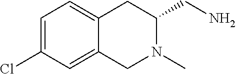

The compound was prepared by general procedure h with mviii (0.1 g, 0.445 mmol) and 2M lithium aluminium hydride in THF (0.67 mL, 1.34 mmol) in anhydrous tetrahydrofuran (10 mL), stirred at 60° C. for 17 h. After workup, the crude was purified by Isco Combi-Flash Companion column chromatography (0-5% methanol with 1% ammonia hydroxide in CH₂Cl₂) to give mix (30 mg, 32%). ¹H NMR (400 MHz, DMSO-d₆) δ 7.20-7.01 (m, 3H), 3.74 (d, 1H), 3.53 (d, 1H), 2.76-2.67 (m, 3H), 2.61-2.52 (m, 1H), 2.49-2.42 (m, 1H), 2.29 (d, 3H).

### Methyl (2S)-2-amino-3-(4-chlorophenyl)propanoate hydrochloride (nii)

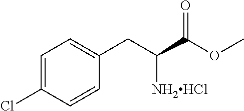

The compound was prepared by general procedure a with (2S)-2-amino-3-(4-chlorophenyl) propanoic acid ni (5 g, 25 mmol) and thionyl chloride (3.63 mL, 50.1 mmol) in anhydrous methanol (100 mL), stirred at 50° C. for 17 h. The desired product nii was obtained as a white solid (6.2 g, 99%). ¹H NMR (400 MHz, DMSO-d₆) δ 8.77-8.56 (m, 3H), 7.40 (d, 2H), 7.28 (d, 2H), 4.34-4.22 (m, 1H), 3.68 (s, 3H), 3.25-3.07 (m, 2H).

### Methyl (2S)-3-(4-chlorophenyl)-2-((ethoxycarbonyl)amino)propanoate (niii)

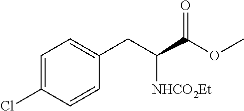

The compound was prepared by general procedure b2 with nii (6.37 g, 27.3 mmol), ethyl chloroformate (4.02 mL, 28.6 mmol) and pyridine (4.83 mL, 60 mmol) in dichloromethane (130 mL), stirred at 0° C. for 2 h. After workup, the crude was purified by Isco Combi-Flash Companion column chromatography (0-30% ethyl acetate in n-hexane) to give niii (7.34 g, 99%). ¹H NMR (400 MHz, DMSO-d₆) δ 7.63 (d, 1H), 7.34 (d, 2H), 7.27 (d, 2H), 4.22 (ddd, 1H), 4.10 (q, 0.2H), 4.00-3.84 (m, 1.8H), 3.62 (s, 3H), 3.02 (dd, 1H), 2.84 (dd, 1H), 1.20 (t, 0.4H), 1.11 (t, 2.6H).

### 2-Ethyl 3-methyl (3S)-7-chloro-3,4-dihydroisoquinoline-2,3(1H)-dicarboxylate (niv)

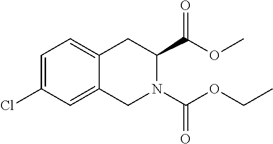 [SMILES: CCOC(=O)N1Cc2cc(Cl)ccc2C[C@H]1C(=O)OC]

The compound was prepared by general procedure c with niii (7 g, 24.5 mmol) and paraformaldehyde (0.81 g, 26.9 mmol) in a mixture of acetic acid (70 mL) and sulfuric acid (30 mL), stirred at room temperature for 17 h. After workup, the crude was purified by Isco Combi-Flash Companion column chromatography (0-30% ethyl acetate in n-hexane) to give niv as a colorless liquid (6.32 g, 87%). ¹H NMR (400 MHz, DMSO-d₆) δ 7.36 (d, 1H), 7.27-7.16 (m, 2H), 4.99 (t, 0.5H), 4.90 (t, 0.5H), 4.63 (dd, 1H), 4.45 (dd, 1H), 4.22-3.98 (m, 2H), 3.56 (s, 1.5H), 3.55 (d, 1.5H), 3.21-3.04 (m, 2H), 1.24 (t, 2H), 1.16 (t, 1H).

### (3S)-7-Chloro-1,2,3,4-tetrahydroisoquinoline-3-carboxylic acid hydrochloride (1:1) (nv)

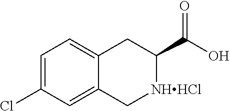

The compound was prepared by general procedure d with niv (6.32 g, 21.2 mmol) in aqueous 8 N hydrogen chloride (100 mL), refluxed for 17 h. The desired product nv hydrochloride (1:1) was obtained as a white solid (3.08 g, 69%). ¹H NMR (400 MHz, DMSO-d₆) δ 9.94 (br s, 2H), 7.41 (s, 1H), 7.34 (dd, 1H), 7.31 (d, 1H). 4.40 (dd, 1H), 4.35 (d, 1H), 4.29 (d, 1H). 3.32 (dd, 1H), 3.09 (dd, 1H).

### Methyl (3S)-7-chloro-1,2,3,4-tetrahydroisoquinoline-3-carboxylate (nvi)

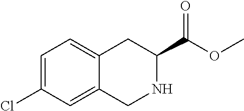

The compound was prepared by general procedure e2 with nv hydrochloride (1:1) (3.08 g, 14.6 mmol) and thionyl chloride (3.69 mL, 50.9 mmol) in anhydrous methanol (100 mL), stirred at 50° C. for 17 h. After workup, the crude was concentrated to give nvi (2.81 g, 86%). ¹H NMR (400 MHz, DMSO-d₆) δ 7.19-7.10 (m, 3H), 3.94 (d, 1H), 3.85 (d, 1H), 3.72-3.62 (m, 4H), 2.93 (dd, 1H), 2.85-2.71 (m, 2H).

### (3S)-7-Chloro-1,2,3,4-tetrahydroisoquinoline-3-carboxamide (nvii)

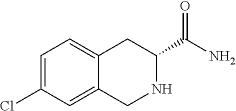 [SMILES: NC(=O)[C@H]1Cc2ccc(Cl)cc2CN1]

The compound was prepared by general procedure f with nvi (2.81 g, 12.5 mmol) in 28-30% aqueous ammonium hydroxide (100 mL), stirred at room temperature for 17 h, to give nvii (2 g, 76%). ¹H NMR (400 MHz, DMSO-d₆) δ 7.33 (br s, 1H), 7.20-7.11 (m, 3H), 7.07 (br s, 1H), 3.93 (d, 1H), 3.84 (d, 1H), 3.35 (dd, 1H), 2.88 (dd, 1H), 2.68 (dd, 1H). 2.59 (br s, 1H).

### (3S)-7-Chloro-2-methyl-1,2,3,4-tetrahydroisoquinoline-3-carboxamide (nviii)

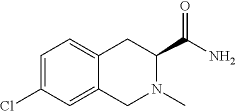

The compound was prepared by general procedure g1 with nvii (2 g, 9.45 mmol), 37% aqueous formaldehyde (1.06 mL, 14.2 mmol), sodium triacetoxyborohydride (4.01 g, 18.9 mmol) and acetic acid (0.540 mL, 9.45 mmol) in acetonitrile (50 mL) and tetrahydrofuran (50 mL), stirred at room temperature for 2 h. After workup, the crude was purified by Isco Combi-Flash Companion column chromatography (0-5% methanol in CH₂Cl₂) to give nviii (2.09 g, 98%). ¹H NMR (400 MHz, DMSO-d₆) δ 7.42 (br s, 1H), 7.21-7.12 (m, 3H), 7.10 (br s, 1H), 3.89 (d, 1H), 3.54 (d, 1H), 3.13 (t, 1H), 2.96-2.78 (m, 2H), 2.33 (s, 3H).

### ((3S)-7-Chloro-2-methyl-1,2,3,4-tetrahydroisoquinolin-3-yl)methanamine (nix)

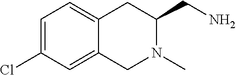

The compound was prepared by general procedure h with nviii (0.2 g, 0.89 mmol) and 2M lithium aluminium hydride in THF (1.34 mL, 2.67 mmol) in anhydrous tetrahydrofuran (9 mL), stirred at 60° C. for 17 h. After workup, the crude was purified by Isco Combi-Flash Companion column chromatography (0-5% methanol with 1% ammonia hydroxide in CH₂Cl₂) to give nix (0.13 g, 69%). ¹H NMR (400 MHz, DMSO-d₆) δ 7.21-7.00 (m, 3H), 3.74 (d, 1H), 3.52 (d, 1H), 2.77-2.63 (m, 3H), 2.62-2.53 (m, 1H), 2.50-2.45 (m, 1H), 2.29 (s, 3H).

### Example 1: N-((2-Methyl-1,2,3,4-tetrahydroisoquinolin-3-yl)methyl)-4-(trifluoromethyl)benzamide (Compound 1)

The compound was prepared by general procedure i1 with Aix (95 mg, 0.539 mmol), 4-(trifluoro-methyl)benzoic acid (102 mg, 0.539 mmol), 1-(3-dimethylaminopropyl)-3-ethylcarbodiimide hydrochloride (207 mg, 1.08 mmol), 1-hydroxybenzotriazole hydrate (148 mg, 1.08 mmol) and N,N-diisopropylethylamine (235 μL, 1.35 mmol) in dichloromethane (10.8 mL), stirred at room temperature for 48 h. After workup, the crude was purified by flash column chromatography (3% methanol in dichloromethane) to give Compound 1 as a pink solid (125 mg, 67%). ¹H NMR (400 MHz, d₆-DMSO) δ 8.70 (t, 1H), 8.03 (d, 2H), 7.85 (d, 2H), 7.15-7.08 (m, 3H), 7.08-7.03 (m, 1H), 3.79 (d, 1H), 3.65-3.53 (m, 2H), 3.28-3.18 (m, 1H), 2.95 (quin, 1H), 2.84 (dd, 1H), 2.69 (dd, 1H), 2.42 (s, 3H); LC-MS (ESI) m/z 349.1 [M+H]⁺.

### Example 2: 4-Ethyl-N-((2-methyl-1,2,3,4-tetrahydroisoquinolin-3-yl)methyl)benzamide (2)

The compound was prepared by general procedure i1 with Aix (95 mg, 0.539 mmol), 4-ethylbenzoic acid (81 mg, 0.539 mmol), 1-(3-dimethylaminopropyl)-3-ethylcarbodiimide hydrochloride (207 mg, 1.08 mmol), 1-hydroxybenzotriazole hydrate (148 mg, 1.08 mmol) and N,N-diisopropylethylamine (235 μL, 1.35 mmol) in dichloromethane (10.8 mL), stirred at room temperature for 48 h. After workup, the crude was purified by flash column chromatography (3% methanol in dichloromethane) to give 2 as a pink syrup (107 mg, 64%). ¹H NMR (400 MHz, d₆-DMSO) δ 8.35 (t, 1H), 7.77 (d, 2H), 7.29 (d, 2H), 7.14-7.08 (m, 3H), 7.07-7.03 (m, 1H), 3.78 (d, 1H), 3.63-3.50 (m, 2H), 3.24-3.12 (m, 1H), 2.92 (quin, 1H), 2.82 (dd, 1H), 2.73-2.60 (m, 3H), 2.41 (s, 3H), 1.19 (t, 3H); LC-MS (ESI) LC-MS (ESI) m/z 309.1 [M+H]⁺.

### Example 3: 4-(Ethylsulfanyl)-N-((2-methyl-1,2,3,4-tetrahydroisoquinolin-3-yl)methyl)benzamide (3)

The compound was prepared by general procedure i1 with Aix (95 mg, 0.539 mmol), 4-(ethylthio)benzoic acid (98 mg, 0.539 mmol), 1-(3-dimethylaminopropyl)-3-ethylcarbodiimide hydrochloride (207 mg, 1.08 mmol), 1-hydroxybenzotriazole hydrate (148 mg, 1.08 mmol) and N,N-diisopropylethylamine (235 μL, 1.35 mmol) in dichloromethane (10.8 mL), stirred at room temperature for 48 h. After workup, the crude was purified by flash column chromatography (3% methanol in dichloromethane) to give 3 as a pink syrup (166 mg, 91%). ¹H NMR (400 MHz, d₆-DMSO) δ 8.40 (t, 1H), 7.78 (d, 2H), 7.35 (d, 2H), 7.15-7.08 (m, 3H), 7.08-7.01 (m, 1H), 3.78 (d, 1H), 3.65-3.48 (m, 2H), 3.24-3.11 (m, 1H), 3.04 (q, 2H), 2.92 (quin, 1H), 2.82 (dd, 1H), 2.68 (dd, 1H), 2.42 (s, 3H), 1.26 (t, 3H); LC-MS (ESI) m/z 341.1 [M+H]⁺.

### Example 4: 3-(3-Fluorophenyl)-N-((2-methyl-1,2,3,4-tetrahydroisoquinolin-3-yl)methyl)propanamide (4)

The compound was prepared by general procedure i1 with Aix (60 mg, 0.34 mmol), 3-(3-fluorophenyl) propanoic acid (57.2 mg, 0.34 mmol), EDCI (84.6 mg, 0.443 mmol), HOBt (50.6 mg, 0.374 mmol) and DIPEA (80.0 μL, 0.511 mmol) in CH₂Cl₂ (5 mL), stirred at room temperature for 16 h. After workup, the crude was purified by Isco Combi-Flash Companion column chromatography (0-5% methanol in CH₂Cl₂) to give 4 (27.3 mg, 25%). ¹H NMR (400 MHz, CDCl₃) δ 7.22-7.18 (m, 1H), 7.17-7.10 (m, 2H), 7.09-6.99 (m, 2H), 6.97 (d, 1H), 6.94-6.83 (m, 2H), 6.03 (br s, 1H), 3.80 (d, 1H), 3.67 (d, 1H), 3.45 (dt, 1H), 3.32 (ddd, 1H), 2.96 (t, 2H), 2.87-2.74 (m, 1H), 2.73-2.60 (m, 2H), 2.49 (t, 2H), 2.32 (s, 3H); LC-MS (ESI) m/z 327.1 [M+H]⁺.

### Example 5: 3-Cyclopentyl-N-((2-methyl-1,2,3,4-tetrahydroisoquinolin-3-yl)methyl)propanamide (5)

The compound was prepared by general procedure i1 with Aix (60 mg, 0.34 mmol), 3-cyclopentylpropanoic acid (49.0 μL, 0.34 mmol), EDCI (84.6 mg, 0.443 mmol), HOBt (50.6 mg, 0.374 mmol) and DIPEA (80.0 μL, 0.511 mmol) in CH₂Cl₂ (5 mL), stirred at room temperature for 17 h. After workup, the crude was purified by Isco Combi-Flash Companion column chromatography (0-5% methanol in CH₂Cl₂) to give 5 (0.1 mg, 97%). ¹H NMR (400 MHz, CDCl₃) δ 7.19-6.98 (m, 4H), 6.48 (br s, 1H), 3.89 (d, 1H), 3.76 (d, 1H), 3.51 (dt, 1H), 3.39 (dt, 1H), 2.99-2.88 (m, 1H), 2.80 (d, 2H), 2.41 (s, 3H), 2.20 (dd, 2H), 1.83-1.68 (m, 3H), 1.68-1.55 (m, 4H), 1.54-1.41 (m, 2H), 1.18-0.99 (m, 2H); LC-MS (ESI) m/z 301.1 [M+H]⁺.

### Example 6: 3-(4-Methylphenyl)-N-((2-methyl-1,2,3,4-tetrahydroisoquinolin-3-yl)methyl)propanamide (6)

The compound was prepared by general procedure i1 with Aix (60 mg, 0.34 mmol), 3-(4-methylphenyl) propanoic acid (55.9 mg, 0.34 mmol), EDCI (84.6 mg, 0.443 mmol), HOBt (50.6 mg, 0.374 mmol) and DIPEA (80.0 μL, 0.511 mmol) in CH₂Cl₂ (5 mL), stirred at room temperature for 17 h. After workup, the crude was purified by Isco Combi-Flash Companion column chromatography (0-5% methanol in CH₂Cl₂) to give 6 (0.1 mg, 91%). ¹H NMR (400 MHz, CDCl₃) δ 7.17-7.11 (m, 2H), 7.10-6.99 (m, 6H), 5.93 (br s, 1H), 3.79 (d, 1H), 3.64 (d, 1H), 3.47-3.27 (m, 2H), 2.92 (t, 2H), 2.81-2.71 (m, 1H), 2.71-2.58 (m, 2H), 2.48 (t, 2H), 2.30 (s, 3H), 2.29 (s, 3H); LC-MS (ESI) m/z 323.1 [M+H]⁺.

### Example 7: 3-(3-Methylphenyl)-N-((2-methyl-1,2,3,4-tetrahydroisoquinolin-3-yl)methyl)propanamide (7)

The compound was prepared by general procedure i1 with Aix (60 mg, 0.34 mmol), 3-(3-methylphenyl) propanoic acid (55.9 mg, 0.34 mmol), EDCI (84.6 mg, 0.443 mmol), HOBt (50.6 mg, 0.374 mmol) and DIPEA (80 μL, 0.511 mmol) in CH₂Cl₂ (5 mL), stirred at room temperature for 17 h. After workup, the crude was purified by Isco Combi-Flash Companion column chromatography (0-5% methanol in CH₂Cl₂) to give 7 (98.7 mg, 90%). ¹H NMR (400 MHz, CDCl₃) δ 7.19-7.10 (m, 3H), 7.08-6.97 (m, 5H), 5.94 (br s, 1H), 3.79 (d, 1H), 3.64 (d, 1H), 3.42 (dt, 1H), 3.32 (dt, 1H), 2.93 (t, 2H), 2.82-2.71 (m, 1H), 2.71-2.58 (m, 2H), 2.49 (t, 2H), 2.30 (s, 3H), 2.29 (s, 3H); LC-MS (ESI) m/z 323.1 [M+H]⁺.

### Example 8: 3-(3-Chlorophenyl)-N-((2-methyl-1,2,3,4-tetrahydroisoquinolin-3-yl)methyl)propanamide (8)

The compound was prepared by general procedure i1 with Aix (60 mg, 0.34 mmol), 3-(3-chlorophenyl) propanoic acid (62.8 mg, 0.34 mmol), EDCI (84.6 mg, 0.443 mmol), HOBt (50.6 mg, 0.374 mmol) and DIPEA (80.0 μL, 0.511 mmol) in CH₂Cl₂ (5 mL), stirred at room temperature for 17 h. After workup, the crude was purified by Isco Combi-Flash Companion column chromatography (0-5% methanol in CH₂Cl₂) to give 8 (90.2 mg, 77%). ¹H NMR (400 MHz, CDCl₃) δ 7.22-6.99 (m, 8H), 5.97 (br s, 1H), 3.79 (d, 1H), 3.65 (d, 1H), 3.43 (dt, 1H), 3.31 (ddd, 1H), 2.94 (t, 2H), 2.84-2.72 (m, 1H), 2.72-2.59 (m, 2H), 2.52-2.45 (m, 2H), 2.32 (S, 3H); LC-MS (ESI) m/z 343.0 [M+H]⁺.

### Example 9: 2-Chloro-5-fluoro-N-((2-methyl-1,2,3,4-tetrahydroisoquinolin-3-yl)methyl)benzamide (9)

The compound was prepared by general procedure i1 with Aix (60 mg, 0.34 mmol), 2-chloro-5-fluorobenzoic acid (59.4 mg, 0.34 mmol), EDCI (84.6 mg, 0.443 mmol), HOBt (50.6 mg, 0.374 mmol) and DIPEA (80.0 μL, 0.511 mmol) in CH₂Cl₂ (5 mL), stirred at room temperature for 17 h. After workup, the crude was purified by Isco Combi-Flash Companion column chromatography (0-5% methanol in CH₂Cl₂) to give 9 (trace). ¹H NMR (400 MHz, CDCl₃) δ 7.41-7.30 (m, 2H), 7.19-7.00 (m, 5H), 6.97 (br s, 1H), 3.89 (d, 1H), 3.78-3.62 (m, 2H), 3.55 (dt, 1H), 3.07-2.94 (m, 1H), 2.85 (d, 2H), 2.43 (s, 3H); LC-MS (ESI) m/z 333.1 [M+H]⁺.

### Example 10: 5-Fluoro-2-methyl-N-((2-methyl-1,2,3,4-tetrahydroisoquinolin-3-yl)methyl)benzamide (10)

The compound was prepared by general procedure i1 with Aix (60 mg, 0.34 mmol), 5-fluoro-2-methylbenzoic acid (52.5 mg, 0.34 mmol), EDCI (84.6 mg, 0.443 mmol), HOBt (50.6 mg, 0.374 mmol) and DIPEA (80 μL, 0.511 mmol) in CH₂Cl₂ (5 mL), stirred at room temperature for 17 h. After workup, the crude was purified by Isco Combi-Flash Companion column chromatography (0-5% methanol in CH₂Cl₂) to give 10 (18.6 mg, 17.5%). ¹H NMR (400 MHz, CDCl₃) δ 7.23-6.91 (m, 8H), 3.97 (d, 1H), 3.87 (d, 1H), 3.75 (dt, 1H), 3.57 (ddd, 1H), 3.18 (br s, 1H), 2.94 (dd, 1H), 2.86 (dd, 1H), 2.50 (s, 3H), 2.40 (s, 3H); LC-MS (ESI) m/z 313.1 [M+H]⁺.

### Example 11: 2,5-Dichloro-N-((2-methyl-1,2,3,4-tetrahydroisoquinolin-3-yl)methyl)benzamide (11)

The compound was prepared by general procedure i1 with Aix (60 mg, 0.34 mmol), 2,5-dichlorobenzoic acid (65 mg, 0.34 mmol), EDCI (84.6 mg, 0.443 mmol), HOBt (50.6 mg, 0.374 mmol) and DIPEA (80.0 μL, 0.511 mmol) in CH₂Cl₂ (5 mL), stirred at room temperature for 17 h. After workup, the crude was purified by Isco Combi-Flash Companion column chromatography (0-5% methanol in CH₂Cl₂) to give 11 (42.4 mg, 36%) as a white solid. ¹H NMR (400 MHz, CDCl₃) δ 8.35 (br s, 1H), 7.53 (d, 1H), 7.36-7.07 (m overlapped with CDCl₃ at 7.24, 6H), 4.19 (d, 1H), 4.10 (d, 1H), 3.88 (dt, 1H), 3.69 (ddd, 1H), 3.53 (br s, 1H), 3.14-2.90 (m, 2H), 2.67 (s, 3H); LC-MS (ESI) m/z 350.1 [M+H]⁺.

### Example 12: N-(((3R)-2-Methyl-1,2,3,4-tetrahydroisoquinolin-3-yl)methyl)-4-(trifluoromethyl)benzamide (12)

The compound was prepared by general procedure i2 with Bix (78 mg, 0.443 mmol), 4-(Trifluoromethyl)benzoyl chloride (0.072 mL, 0.487 mmol) and triethylamine (0.074 mL, 0.532 mmol) in dichloromethane (4.4 mL)), stirred at room temperature for 18 h. After workup, the crude was purified by flash column chromatography (2% methanol in dichloromethane) to give 12 as a beige solid (57 mg, 37%). ¹H NMR (300 MHz, d₆-DMSO) δ 8.69 (t, 1H), 8.03 (d, 2H), 7.85 (d, 2H), 7.16-7.02 (m, 4H), 3.80 (d, 1H), 3.67-3.52 (m, 2H), 3.28-3.14 (m overlapped with H₂O, 1H), 2.96 (quin, 1H), 2.85 (dd, 1H), 2.69 (dd, 1H), 2.43 (s, 3H); LC-MS (ESI) m/z 349.1 [M+H]⁺.

### Example 13: 4-(Ethylsulfanyl)-N-(((3R)-2-methyl-1,2,3,4-tetrahydroisoquinolin-3-yl)methyl)benzamide (13)

The compound was prepared by general procedure i1 with Bix (60 mg, 0.34 mmol), 4-(ethylthio)benzoic acid (62 mg, 0.34 mmol), 1-(3-dimethylaminopropyl)-3-ethylcarbodiimide hydrochloride (130 mg, 0.68 mmol), 1-hydroxybenzotriazole hydrate (92 mg, 0.68 mmol) and N,N-diisopropylethylamine (210 μL, 0.742 mmol) in dichloromethane (6.8 mL), stirred at room temperature for 18 h. After workup, the crude was purified by flash column chromatography (2% methanol in dichloromethane) to give 13 as a white solid (74 mg, 64%). ¹H NMR (300 MHz, d₆-DMSO) δ 8.39 (t, 1H), 7.78 (d, 2H), 7.35 (d, 2H), 7.14-7.07 (m, 3H), 7.07-7.02 (m, 1H), 3.78 (d, 1H), 3.64-3.48 (m, 2H), 3.26-3.11 (m, 1H), 3.05 (q, 2H), 2.91 (quin, 1H), 2.82 (dd, 1H), 2.67 (dd, 1H), 2.41 (s, 3H), 1.26 (t, 3H); LC-MS (ESI) m/z 341.1 [M+H]⁺.

### Example 14: 3,4-Dichloro-N-(((3R)-2-methyl-1,2,3,4-tetrahydroisoquinolin-3-yl)methyl)benzamide (14)

The compound was prepared by general procedure i1 with Bix (60 mg, 0.34 mmol), 3,4-dichlorobenzoic acid (65 mg, 0.34 mmol), 1-(3-dimethylaminopropyl)-3-ethylcarbodiimide hydrochloride (130 mg, 0.68 mmol), 1-hydroxybenzotriazole hydrate (92 mg, 0.68 mmol) and N,N-diisopropylethylamine (210 μL, 0.742 mmol) in dichloromethane (6.8 mL), stirred at room temperature for 18 h. After workup, the crude was purified by flash column chromatography (2% methanol in dichloromethane) to give 14 as a yellow syrup (102 mg, 86%). ¹H NMR (300 MHz, d₆-DMSO) δ 8.64 (t, 1H), 8.07 (d, 1H), 7.82 (dd, 1H), 7.75 (d, 1H), 7.17-7.08 (m, 3H), 7.08-7.01 (m, 1H), 3.78 (d, 1H), 3.65-3.50 (m, 2H), 3.26-3.13 (m, 1H), 2.93 (quin, 1H), 2.83 (dd, 1H), 2.67 (dd, 1H), 2.41 (s, 3H); LC-MS (ESI) m/z 348.9 [M+H]⁺.

### Example 15: 3-(3-Fluorophenyl)-N-(((3R)-2-methyl-1,2,3,4-tetrahydroisoquinolin-3-yl)methyl)propanamide (15)

The compound was prepared by general procedure i1 with Bix (90 mg, 0.511 mmol), 3-(3-fluorophenyl) propionic acid (82 mg, 0.485 mmol), 1-(3-dimethylaminopropyl)-3-ethylcarbodiimide hydrochloride (147 mg, 0.766 mmol), 1-hydroxybenzotriazole hydrate (104 mg, 0.766 mmol) and N,N-diisopropylethylamine (311 μL, 1.79 mmol) in dichloromethane (10 mL), stirred at room temperature for 18 h. After workup, the crude was purified by flash column chromatography (3% methanol in dichloromethane) to give 15 as a yellow syrup (72 mg, 46%). ¹H NMR (300 MHz, d₆-DMSO) δ 7.82 (s, 1H), 7.29 (q, 1H), 7.15-6.93 (m, 7H), 3.73 (d, 1H), 3.51 (d, 1H), 3.31-3.22 (m overlapped with H₂O, 1H), 3.02 (dt, 1H), 2.83 (t, 2H), 2.71-2.53 (m, 3H), 2.42 (t, 2H), 2.30 (s, 1H); LC-MS (ESI) m/z 327.2 [M+H]⁺.

### Example 16: N-(((3R)-2-Methyl-1,2,3,4-tetrahydroisoquinolin-3-yl)methyl)-3-phenylpropanamide (16)

The compound was prepared by general procedure i1 with Bix (90 mg, 0.511 mmol), 3-phenylpropionic acid (73 mg, 0.485 mmol), 1-(3-dimethylaminopropyl)-3-ethylcarbodiimide hydrochloride (147 mg, 0.766 mmol), 1-hydroxybenzotriazole hydrate (104 mg, 0.766 mmol) and N,N-diisopropylethylamine (311 μL, 1.79 mmol) in dichloromethane (10 mL), stirred at room temperature for 18 h. After workup, the crude was purified by flash column chromatography (3% methanol in dichloromethane) to give 16 as a beige solid (68 mg, 48%). ¹H NMR (300 MHz, d₆-DMSO) δ 7.82 (t, 1H), 7.30-7.14 (m, 5H), 7.14-6.99 (m, 4H), 3.73 (d, 1H), 3.52 (d, 1H), 3.31-3.27 (m overlapped with H₂O, 1H), 3.03 (dt, 1H), 2.81 (t, 2H), 2.72-2.52 (m, 3H), 2.40 (t, 2H), 2.31 (s, 3H); LC-MS (ESI) m/z 309.2 [M+H]⁺.

### Example 17: 3-Cyclohexyl-N-(((3R)-2-methyl-1,2,3,4-tetrahydroisoquinolin-3-yl)methyl)propanamide (17)

The compound was prepared by general procedure i1 with Bix (90 mg, 0.511 mmol), 3-cyclohexylpropionic acid (76 μL, 0.485 mmol), 1-(3-dimethylaminopropyl)-3-ethylcarbodiimide hydrochloride (147 mg, 0.766 mmol), 1-hydroxybenzotriazole hydrate (104 mg, 0.766 mmol) and N,N-diisopropylethylamine (311 μL, 1.79 mmol) in dichloromethane (10 mL), stirred at room temperature for 18 h. After workup, the crude was purified by flash column chromatography (3% methanol in dichloromethane) to give 17 as a beige solid (76 mg, 50%). ¹H NMR (300 MHz, d₆-DMSO) δ 7.77 (t, 1H), 7.16-6.99 (m, 4H), 3.75 (d, 1H), 3.54 (d, 1H), 3.32-3.23 (m overlapped with H₂O, 1H), 3.10-2.97 (m 1H), 2.81-2.56 (m, 3H), 2.35 (s, 3H), 2.09 (t, 2H), 1.74-1.53 (m, 5H), 1.38 (q, 2H), 1.27-1.04 (m, 4H), 0.92-0.78 (m, 2H); LC-MS (ESI) m/z 315.2 [M+H]⁺.

### Example 18: 3-(Cyclobutyl-N-(((3R)-2-methyl-1,2,3,4-tetrahydroisoquinolin-3-yl)methyl)propanamide (18)

The compound was prepared by general procedure i1 with Bix (90 mg, 0.511 mmol), 3-cyclobutylpropanoic acid (58 μL, 0.485 mmol), 1-(3-dimethylaminopropyl)-3-ethylcarbodiimide hydrochloride (147 mg, 0.766 mmol), 1-hydroxybenzotriazole hydrate (104 mg, 0.766 mmol) and N,N-diisopropylethylamine (311 μL, 1.79 mmol) in dichloromethane (10 mL), stirred at room temperature for 18 h. After workup, the crude was purified by flash column chromatography (3% methanol in dichloromethane) to give 18 as a yellow syrup (54 mg, 39%). ¹H NMR (300 MHz, d₆-DMSO) δ 7.76 (t, 1H), 7.17-6.99 (m, 4H), 3.75 (d, 1H), 3.54 (d, 1H), 3.41-3.25 (m overlapped with H₂O, 1H), 3.09-2.95 (m, 1H), 2.81-2.55 (m, 3H), 2.34 (s, 3H), 2.19 (hept, 1H), 2.05-1.90 (m, 4H), 1.87-1.68 (m, 2H), 1.64-1.48 (m, 4H); LC-MS (ESI) m/z 287.2 [M+H]⁺.

### Example 19: (2R)—N-{[(3R)-2-Methyl-1,2,3,4-tetrahydroisoquinolin-3-yl]methyl}-1,2,3,4-tetrahydronaphthalene-2-carboxamide (19)

The compound was prepared by general procedure i1 with Bix (0.4 mg, 0.227 mmol), (R)-1,2,3,4-tetrahydronaphthalene-2-carboxylic acid (40 mg, 0.227 mmol), 1-(3-dimethylaminopropyl)-3-ethylcarbodiimide hydrochloride (65.3 mg, 0.34 mmol), 1-hydroxybenzotriazole hydrate (46 mg, 0.34 mmol) and N,N-diisopropylethylamine (98.8 μL, 0.567 mmol) in dichloromethane (2.30 mL), stirred at room temperature for 16 h. After workup, the crude was purified by flash column chromatography (SiO₂, dichloromethane/methanol=98/2) to give 19 as a yellow liquid (61.2 mg, 81%). [α]_{D}²⁴=+4.26° (c=1, MeOH); ¹H NMR (CDCl₃, 400 MHz) δ 7.18-7.03 (m, 8H), 6.29 (br s, 1H), 3.84 (d, 1H), 3.71 (d, 1H), 3.51 (dt, 1H), 3.40 (dt, 1H), 3.03 (dd, 1H), 2.97-2.70 (m, 6H), 2.54 (dddd, 1H), 2.39 (s, 3H), 2.13-2.06 (m, 1H), 1.96-1.84 (m, 1H); LC-MS (ESI) m/z 335.1 [M+H]⁺.

### Example 20: (2S)—N-{[(3R)-2-Methyl-1,2,3,4-tetrahydroisoquinolin-3-yl]methyl}-1,2,3,4-tetrahydronaphthalene-2-carboxamide (20)

The compound was prepared by general procedure i1 with Bix (0.4 mg, 0.227 mmol), (S)-1,2,3,4-tetrahydronaphthalene-2-carboxylic acid (40 mg, 0.227 mmol), 1-(3-dimethylaminopropyl)-3-ethylcarbodiimide hydrochloride (65.3 mg, 0.34 mmol), 1-hydroxybenzotriazole hydrate (46 mg, 0.34 mmol) and N,N-diisopropylethylamine (98.8 μL, 0.567 mmol) in dichloromethane (2.30 mL), stirred at room temperature for 16 h. After workup, the crude was purified by flash column chromatography (SiO₂, dichloromethane/methanol=98/2) to give 20 as a yellow liquid (62.4 mg, 82%). [α]_{D}²⁴=+0.74° (c=1, MeOH); ¹H NMR (CDCl₃, 400 MHz) δ 7.19-7.03 (m, 8H), 6.32 (br s, 1H), 3.86 (d, 1H), 3.71 (d, 1H), 3.50 (dt, 1H), 3.42 (dt, 1H), 3.05 (dd, 1H), 2.97-2.72 (m, 6H), 2.54 (dddd, 1H), 2.40 (s, 3H), 2.14-2.06 (m, 1H), 1.97-1.85 (m, 1H); LC-MS (ESI) m/z 335.2 [M+H]⁺.

### Example 21: N-{[(3R)-2-Methyl-1,2,3,4-tetrahydroisoquinolin-3-yl]methyl}-2,3-dihydro-1H-indene-2-carboxamide (21)

The compound was prepared by general procedure i1 with Bix (0.525 g, 2.98 mmol), 2-indanecarboxylic acid (0.483 g, 2.98 mmol), 1-(3-dimethylaminopropyl)-3-ethylcarbodiimide hydrochloride (0.857 g, 4.47 mmol), 1-hydroxybenzotriazole hydrate (0.604 g, 4.47 mmol) and N,N-diisopropylethylamine (1.31 mL, 7.45 mmol) in dichloromethane (29.8 mL), stirred at room temperature for 16 h. After workup, the crude was purified by flash column (SiO₂, dichloromethane/methanol=97/3) to give 21 as a white solid (0.782 g, 82%). [α]_{D}²⁴=+57.57° (c=1, MeOH); ¹H NMR (CDCl₃, 400 MHz) δ 7.25-7.03 (m, 8H), 6.23 (br s, 1H), 3.86 (d, 1H), 3.73 (d, 1H), 3.52 (dt, 1H), 3.40 (ddd, 1H), 3.28-3.11 (m, 5H), 2.95-2.87 (m, 1H), 2.85-2.71 (m, 2H), 2.40 (s, 3H); LC-MS (ESI) m/z 321.2 [M+H]⁺.

### Example 22: N-(((3S)-2-Methyl-1,2,3,4-tetrahydroisoquinolin-3-yl)methyl)-4-(trifluoromethyl)benzamide (22)

The compound was prepared by general procedure i1 with Cix (85 mg, 0.482 mmol), 4-(trifluoromethyl)benzoic acid (92 mg, 0.482 mmol), 1-(3-dimethylaminopropyl)-3-ethylcarbodiimide hydrochloride (185 mg, 0.964 mmol), 1-hydroxybenzotriazole hydrate (130 mg, 0.964 mmol) and N,N-diisopropylethylamine (293 μL, 1.69 mmol) in dichloromethane (9.6 mL), stirred at room temperature for 18 h. After workup, the crude was purified by flash column chromatography (2% methanol in dichloromethane) to give 22 as a white solid (110 mg, 65%). ¹H NMR (300 MHz, d₆-DMSO) δ 8.68 (t, 1H), 8.03 (d, 2H), 7.85 (d, 2H), 7.16-7.08 (m, 3H), 7.08-7.02 (m, 1H), 3.78 (d, 1H), 3.65-3.52 (m, 2H), 3.28-3.16 (m, 1H), 2.94 (quin, 1H), 2.84 (dd, 1H), 2.69 (dd, 1H), 2.42 (s, 3H); LC-MS (ESI) m/z 349.1 [M+H]⁺.

### Example 23: 4-Ethyl-N-(((3S)-2-methyl-1,2,3,4-tetrahydroisoquinolin-3-yl)methyl)benzamide (23)

The compound was prepared by general procedure i1 with Cix (85 mg, 0.482 mmol), 4-ethylbenzoic acid (72 mg, 0.482 mmol), 1-(3-dimethylaminopropyl)-3-ethylcarbodiimide hydrochloride (185 mg, 0.964 mmol), 1-hydroxybenzotriazole hydrate (130 mg, 0.964 mmol) and N,N-diisopropylethylamine (293 μL, 1.69 mmol) in dichloromethane (9.6 mL), stirred at room temperature for 18 h. After workup, the crude was purified by flash column chromatography (2% methanol in dichloromethane) to give 23 as a white solid (102 mg, 68%). ¹H NMR (300 MHz, d₆-DMSO) δ 8.35 (t, 1H), 7.77 (d, 2H), 7.29 (d, 2H), 7.17-7.07 (m, 3H), 7.07-7.01 (m, 1H), 3.78 (d, 1H), 3.65-3.49 (m, 2H), 3.26-3.12 (m, 1H), 2.93 (quin, 1H), 2.82 (dd, 1H), 2.73-2.59 (m, 3H), 2.42 (s, 3H), 1.19 (t, 3H); LC-MS (ESI) m/z 309.1 [M+H]⁺.

### Example 24: 4-(Ethylsulfanyl)-N-(((3S)-2-methyl-1,2,3,4-tetrahydroisoquinolin-3-yl)methyl)benzamide (24)

The compound was prepared by general procedure i1 with Cix (85 mg, 0.482 mmol), 4-(ethylthio)benzoic acid (88 mg, 0.482 mmol), 1-(3-dimethylaminopropyl)-3-ethylcarbodiimide hydrochloride (185 mg, 0.964 mmol), 1-hydroxybenzotriazole hydrate (130 mg, 0.964 mmol) and N,N-diisopropylethylamine (293 μL, 1.69 mmol) in dichloromethane (9.6 mL), stirred at room temperature for 18 h. After workup, the crude was purified by flash column chromatography (2% methanol in dichloromethane) to give 24 as a white solid (102 mg, 68%). ¹H NMR (300 MHz, d₆-DMSO) δ 8.39 (t, 1H), 7.78 (d, 2H), 7.35 (d, 2H), 7.15-7.07 (m, 3H), 7.07-7.01 (m, 1H), 3.77 (d, 1H), 3.64-3.49 (m, 2H), 3.25-3.12 (m, 1H), 3.05 (q, 2H), 2.91 (quin, 1H), 2.82 (dd, 1H), 2.67 (dd, 1H), 2.41 (s, 3H), 1.26 (t, 3H); LC-MS (ESI) m/z 341.1 [M+H]⁺.

### Example 25: 3,4-Dichloro-N-(((3S)-2-methyl-1,2,3,4-tetrahydroisoquinolin-3-yl)methyl)benzamide (25)

The compound was prepared by general procedure i1 with Cix (85 mg, 0.482 mmol), 3,4-dichlorobenzoic acid (92 mg, 0.482 mmol), 1-(3-dimethylaminopropyl)-3-ethylcarbodiimide hydrochloride (185 mg, 0.964 mmol), 1-hydroxybenzotriazole hydrate (130 mg, 0.964 mmol) and N,N-diisopropylethylamine (293 μL, 1.69 mmol) in dichloromethane (9.6 mL), stirred at room temperature for 18 h. After workup, the crude was purified by flash column chromatography (2% methanol in dichloromethane) to give 25 as a yellow syrup (146 mg, 87%). ¹H NMR (300 MHz, d₆-DMSO) δ 8.64 (t, 1H), 8.07 (d, 1H), 7.82 (dd, 1H), 7.75 (d, 1H), 7.15-7.08 (m, 3H), 7.08-7.02 (m, 1H), 3.77 (d, 1H), 3.64-3.50 (m, 2H), 3.26-3.14 (m, 1H), 2.92 (quin, 1H), 2.83 (dd, 1H), 2.67 (dd, 1H), 2.41 (s, 3H); LC-MS (ESI) m/z 349.0 [M+H]⁺.

### Example 26: 3-(3-Fluorophenyl)-N-{[(3S)-2-methyl-1,2,3,4-tetrahydroisoquinolin-3-yl]methyl}propanamide (26)

The compound was prepared by general procedure i1 with Cix (60 mg, 0.34 mmol), 3-(3-fluorophenyl) propionic acid (40.1 mg, 0.238 mmol), 1-(3-dimethylaminopropyl)-3-ethylcarbodiimide hydrochloride (0.131 g, 0.681 mmol), 1-hydroxybenzotriazole hydrate (92 mg, 0.681 mmol) and N,N-diisopropylethylamine (0.208 mL, 1.19 mmol) in dichloromethane (4.90 mL), stirred at room temperature for 16 h. After workup, the crude was purified by flash column chromatography (SiO₂, dichloromethane/methanol=98/2) to give 26 as a brown liquid (55.3 mg, 71%). ¹H NMR (CDCl₃, 300 MHz) δ 7.26-7.11 (m, overlapped with CDCl₃, 3H), 7.10-6.95 (m, 3H), 6.94-6.82 (m, 2H), 6.14 (br s, 1H), 3.82 (d, 1H), 3.68 (d, 1H), 3.45 (dt, 1H), 3.32 (dt, 1H), 2.96 (t, 2H), 2.87-2.75 (m, 1H), 2.73-2.59 (m, 2H), 2.49 (t, overlapped with H₂O, 2H), 2.33 (s, 3H); LC-MS (ESI) m/z 327.2 [M+H]⁺.

### Example 27: N-{[(3S)-2-Methyl-1,2,3,4-tetrahydroisoquinolin-3-yl]methyl}-3-phenylpropanamide (27)

The compound was prepared by general procedure i1 with Cix (64.2 mg, 0.364 mmol), 3-phenylpropionic acid (38.3 mg, 0.255 mmol), 1-(3-dimethylaminopropyl)-3-ethylcarbodiimide hydrochloride (0.140 g, 0.728 mmol), 1-hydroxybenzotriazole hydrate (98.4 mg, 0.728 mmol) and N,N-diisopropylethylamine (0.222 mL, 1.27 mmol) in dichloromethane (5.20 mL), stirred at room temperature for 16 h. After workup, the crude was purified by flash column chromatography (SiO₂, dichloromethane/methanol=98/2) to give 27 as a brown solid (48 mg, 61%). ¹H NMR (CDCl₃, 300 MHz) δ 7.35-7.15 (m, 7H), 7.14-7.02 (m, 2H), 6.14 (br s, 1H), 3.85 (d, 1H), 3.71 (d, 1H), 3.48 (dt, 1H), 3.37 (dt, 1H), 3.01 (t, 2H), 2.89-2.78 (m, 1H), 2.76-2.62 (m, 2H), 2.55 (t, overlapped with H₂O, 2H), 2.36 (s, 3H); LC-MS (ESI) m/z 309.2 [M+H]⁺.

### Example 28: 3-(Cyclohexyl-N-{[(3S)-2-methyl-1,2,3,4-tetrahydroisoquinolin-3-yl]methyl}propanamide (28)

The compound was prepared by general procedure i1 with Cix (60 mg, 0.34 mmol), 3-cyclohexylpropionic acid (43.7 μL, 0.255 mmol), 1-(3-dimethylaminopropyl)-3-ethylcarbodiimide hydrochloride (0.131 g, 0.681 mmol), 1-hydroxybenzotriazole hydrate (92 mg, 0.681 mmol) and N,N-diisopropylethylamine (0.208 mL, 1.19 mmol) in dichloromethane (4.90 mL), stirred at room temperature for 16 h. After workup, the crude was purified by flash column chromatography (SiO₂, dichloromethane/methanol=98/2) to give 28 as a brown solid (62.6 mg, 78%). ¹H NMR (CDCl₃, 300 MHz) δ 7.18-6.98 (m, 4H), 6.20 (br s, 1H), 3.86 (d, 1H), 3.71 (d, 1H), 3.47 (dt, 1H), 3.36 (dt, 1H), 2.93-2.81 (m, 1H), 2.80-2.71 (m, 2H), 2.39 (s, 3H), 2.19 (dd, 2H), 1.75-1.58 (m, 5H), 1.51 (q, 2H), 1.30-1.17 (m, 4H), 0.96-0.79 (m, 2H); LC-MS (ESI) m/z 315.3 [M+H]⁺.

### Example 29: 3-Cyclobutyl-N-{[(3S)-2-methyl-1,2,3,4-tetrahydroisoquinolin-3-yl]methyl}propanamide (29)

The compound was prepared by general procedure i1 Cix (70 mg, 0.397 mmol), 3-cyclobutylpropanoic acid (36 μL, 0.298 mmol), 1-(3-dimethylaminopropyl)-3-ethylcarbodiimide hydrochloride (0.152 g, 0.794 mmol), 1-hydroxybenzotriazole hydrate (0.107 g, 0.794 mmol) and N,N-diisopropylethylamine (0.242 mL, 1.39 mmol) in dichloromethane (6.50 mL), stirred at room temperature for 16 h. After workup, the crude was purified by flash column chromatography (SiO₂, dichloromethane/methanol=98/2) to give 29 as a yellow liquid (63.3 mg, 56%). ¹H NMR (CDCl₃, 400 MHz) δ 7.21-7.14 (m, 2H), 7.12-7.09 (m, 1H), 7.07-7.03 (m, 1H), 6.60 (br s, 1H), 3.93 (d, 1H), 3.81 (d, 1H), 3.54 (dt, 1H), 3.40 (ddd, overlapped with H₂O, 1H), 3.05-2.97 (m, 1H), 2.88-2.75 (m, 2H), 2.45 (s, 3H) 2.31-2.15 (m, 2H), 2.13-1.98 (m, 3H), 1.88-1.75 (m, 2H), 1.71 (dd, 2H), 1.67-1.53 (m, 2H); LC-MS (ESI) m/z 287.2 [M+H]⁺.

### Example 30: 3-(Cyclopropyl-N-{[(3S)-2-methyl-1,2,3,4-tetrahydroisoquinolin-3-yl]methyl}propanamide (30)

The compound was prepared by general procedure i1 with Cix (68.5 mg, 0.389 mmol), 3-cyclopropylpropanoic acid (29.7 μL, 0.292 mmol), 1-(3-dimethylaminopropyl)-3-ethylcarbodiimide hydrochloride (0.148 g, 0.777 mmol), 1-hydroxybenzotriazole hydrate (0.105 g, 0.777 mmol) and N,N-diisopropylethylamine (0.237 mL, 1.36 mmol) in dichloromethane (5.60 mL), stirred at room temperature for 16 h. After workup, the crude was purified by flash column chromatography (SiO₂, dichloromethane/methanol=98/2) to give 30 as a brown liquid (39.7 mg, 50%). ¹H NMR (CDCl₃, 300 MHz) δ 7.20-7.00 (m, 4H), 6.40 (br s, 1H), 3.89 (d, 1H), 3.75 (d, overlapped with H₂O, 1H), 3.51 (dt, 1H), 3.38 (dt, 1H), 3.00-2.88 (m, 1H), 2.83-2.74 (m, 2H), 2.41 (s, 3H), 2.29 (t, 2H), 1.53 (dd, 2H), 0.75-0.61 (m, 1H), 0.44-0.36 (m, 2H), 0.08-0.01 (m, 2H); LC-MS (ESI) m/z 273.2 [M+H]⁺.

### Example 31: N-(((3R)-2,5-Dimethyl-1,2,3,4-tetrahydroisoquinolin-3-yl)methyl)-4-ethylbenzamide (31)

The compound was prepared by general procedure i1 with Dix (0.1 g, 0.526 mmol), 4-ethylbenzoic acid (78.9 mg, 0.526 mmol), EDCI (0.130 g, 0.683 mmol), HOBt (78.1 mg, 0.578 mmol) and DIPEA (0.13 mL, 0.788 mmol) in CH₂Cl₂ (5 mL), stirred at room temperature for 17 h. After workup, the crude was purified by Isco Combi-Flash Companion column chromatography (0-3% methanol in CH₂Cl₂) to give 31 (0.14 g, 83%). ¹H NMR (400 MHz, d₆-DMSO) δ 8.35 (br t, 1H), 7.75 (d, 2H), 7.29 (d, 2H), 7.05-6.95 (m, 2H), 6.87 (d, 1H), 3.75 (d, 1H), 3.62-3.50 (m, 2H), 3.22 (dt, 1H), 2.96-2.86 (m, 1H), 2.72-2.59 (m, 3H), 2.55-2.50 (m overlapped with d₆-DMSO, 1H), 2.38 (s, 3H), 2.15 (s, 3H), 1.18 (t, 3H); LC-MS (ESI) m/z 323.2 [M+H]⁺.

### Example 32: 3,4-Dichloro-N-(((3R)-2,5-dimethyl-1,2,3,4-tetrahydroisoquinolin-3-yl)methyl)benzamide (32)

The compound was prepared by general procedure i2 with Dix (0.1 g, 0.526 mmol), 3,4-dichlorobenzoyl chloride (0.11 g, 0.526 mmol) and triethylamine (88.0 μL, 0.631 mmol) in CH₂Cl₂ (5 mL), stirred at room temperature for 17 h. After workup, the crude was purified by Isco Combi-Flash Companion column chromatography (0-3% methanol in CH₂Cl₂) to give 32 (160 mg, 84%). ¹H NMR (400 MHz, d₆-DMSO) δ 8.64 (br t, 1H), 8.05 (d, 1H), 7.81 (dd, 1H), 7.75 (d, 1H), 7.06-6.94 (m, 2H), 6.87 (d, 1H), 3.75 (d, 1H), 3.63-3.51 (m, 2H), 3.24 (dt, 1H), 2.92 (ddd, 1H), 2.65 (dd, 1H), 2.56-2.50 (m overlapped with d₆-DMSO, 1H), 2.37 (s, 3H), 2.15 (s, 3H); LC-MS (ESI) m/z 364.1 [M+H]⁺.

### Example 33: N-(((3R)-2,5-Dimethyl-1,2,3,4-tetrahydroisoquinolin-3-yl)methyl)-3-(3-fluorophenyl)propanamide (33)

The compound was prepared by general procedure i1 with Dix (97.7 mg, 0.513 mmol), 3-(3-fluorophenyl) propanoic acid (86.3 mg, 0.513 mmol), EDCI (0.128 g, 0.667 mmol), HOBt (76.3 mg, 0.565 mmol) and DIPEA (0.127 mL, 0.77 mmol) in CH₂Cl₂ (5 mL), stirred at room temperature for 17 h. After workup, the crude was purified by Isco Combi-Flash Companion column chromatography (0-5% methanol in CH₂Cl₂) to give 33 (0.12 g, 69%). ¹H NMR (400 MHz, d₆-DMSO) δ 7.82 (br t, 1H), 7.32-7.22 (m, 1H), 7.08-6.91 (m, 5H), 6.85 (d, 1H), 3.70 (d, 1H), 3.48 (d, 1H), 3.38-3.27 (m overlapped with H₂O, 1H), 3.12-3.01 (m, 1H), 2.83 (t, 2H), 2.65-2.56 (m, 1H), 2.56-2.50 (m overlapped with do-DMSO, 1H), 2.46-2.34 (t, 3H), 2.27 (s, 3H), 2.12 (s, 3H); LC-MS (ESI) m/z 341.2 [M+H]⁺.

### Example 34: N-(((3R)-2,5-Dimethyl-1,2,3,4-tetrahydroisoquinolin-3-yl)methyl)-3-phenylpropanamide (34)

The compound was prepared by general procedure i1 with Dix (0.1 g, 0.526 mmol), 3-phenylpropanoic acid (78.9 mg, 0.526 mmol), EDCI (0.130 g, 0.683 mmol), HOBt (78.1 mg, 0.578 mmol) and DIPEA (0.13 mL, 0.788 mmol) in CH₂Cl₂ (5 mL), stirred at room temperature for 17 h. After workup, the crude was purified by Isco Combi-Flash Companion column chromatography (0-5% methanol in CH₂Cl₂) to give 34 (0.12 g, 71%). ¹H NMR (400 MHz, d₆-DMSO) δ 7.81 (br t, 1H), 7.28-7.10 (m, 5H), 7.03-6.94 (m, 2H), 6.85 (d, 1H), 3.70 (d, 1H), 3.48 (d, 1H), 3.38-3.30 (m overlapped with H₂O, 1H), 3.12-3.02 (m, 1H), 2.80 (t, 2H), 2.66-2.50 (m overlapped with d₆-DMSO, 3H), 2.40 (t, 2H), 2.28 (s, 3H), 2.12 (s, 3H); LC-MS (ESI) m/z 323.2 [M+H]⁺.

### Example 35: 3-Cyclohexyl-N-(((3R)-2,5-dimethyl-1,2,3,4-tetrahydroisoquinolin-3-yl)methyl)propanamide (35)

The compound was prepared by general procedure i1 with Dix (99.6 mg, 0.523 mmol), 3-cyclohexylpropanoic acid (55.9 μL, 0.523 mmol), EDCI (0.13 g, 0.68 mmol), HOBt (77.8 mg, 0.576 mmol) and DIPEA (0.130 mL, 0.785 mmol) in CH₂Cl₂ (5 mL), stirred at room temperature for 17 h. After workup, the crude was purified by Isco Combi-Flash Companion column chromatography (0-5% methanol in CH₂Cl₂) to give 35 (0.13 g, 76%). ¹H NMR (400 MHz, d₆-DMSO) δ 7.78 (br t, 1H), 7.06-6.94 (m, 2H), 6.86 (d, 1H), 3.72 (d, 1H), 3.50 (d, 1H), 3.40-3.34 (m overlapped with H₂O, 1H), 3.09 (dt, 1H), 2.71-2.53 (m, 2H), 2.50-2.41 (m overlapped with do-DMSO, 1H), 2.32 (s, 3H), 2.16 (s, 3H), 2.10 (t, 2H), 1.73-1.56 (m, 5H), 1.39 (q, 2H), 1.20-1.08 (m, 4H), 0.90-0.80 (m, 2H); LC-MS (ESI) m/z 329.3 [M+H]⁺.

### Example 36: N-(((3R)-2,5-Dimethyl-1,2,3,4-tetrahydroisoquinolin-3-yl)methyl)-2,3-dihydro-1H-indene-2-carboxamide (36)

The compound was prepared by general procedure i1 with Dix (0.1 g, 0.526 mmol), 2,3-dihydro-1H-indene-2-carboxylic acid (85.2 mg, 0.526 mmol), EDCI (0.13 g, 0.683 mmol), HOBt (78.1 mg, 0.578 mmol) and DIPEA (0.13 mL, 0.788 mmol) in CH₂Cl₂ (5 mL), stirred at room temperature for 16 h. After workup, the crude was purified by Isco Combi-Flash Companion column chromatography (0-3% methanol in CH₂Cl₂) to give 36 (0.13 g, 74%). ¹H NMR (400 MHz, d₆-DMSO) δ 7.95 (br t, 1H), 7.21-7.14 (m, 2H), 7.13-7.06 (m, 2H), 7.05-6.95 (m, 2H), 6.86 (d, 1H), 3.74 (d, 1H), 3.51 (d, 1H), 3.40 (dt, 1H), 3.30-3.09 (m, 2H), 3.05 (d, 2H), 3.03 (d, 2H), 2.76-2.56 (m, 2H), 2.52-2.43 (m overlapped with d₆-DMSO, 1H), 2.33 (s, 3H), 2.16 (s, 3H); LC-MS (ESI) m/z 335.2 [M+H]⁺.

### Example 37: N-(((3S)-2,5-Dimethyl-1,2,3,4-tetrahydroisoquinolin-3-yl)methyl)-3-(3-fluorophenyl)propanamide (37)

The compound was prepared by general procedure i1 with Eix (61.5 mg, 0.323 mmol), 3-(3-fluorophenyl) propanoic acid (54.3 mg, 0.323 mmol), EDCI (80.3 mg, 0.42 mmol), HOBt (48 mg, 0.356 mmol) and DIPEA (80 μL, 0.485 mmol) in CH₂Cl₂ (5 mL), stirred at room temperature for 16 h. After workup, the crude was purified by Isco Combi-Flash Companion column chromatography (0-5% methanol in CH₂Cl₂) to give 37 (92.4 mg, 84%). ¹H NMR (400 MHz, d₆-DMSO) δ 7.78 (br t, 1H), 7.30-7.24 (m, 1H), 7.08-6.91 (m, 5H), 6.85 (d, 1H), 3.70 (d, 1H), 3.48 (d, 1H), 3.34 (dt, 1H), 3.07 (dt, 1H), 2.83 (t, 2H), 2.69-2.56 (m, 1H), 2.58-2.48 (m overlapped with d₆-DMSO, 1H), 2.47-2.34 (m, 3H), 2.28 (s, 3H), 2.12 (s, 3H); LC-MS (ESI) m/z 341.2 [M+H]⁺.

### Example 38: N-(((3S)-2,5-Dimethyl-1,2,3,4-tetrahydroisoquinolin-3-yl)methyl)-3-phenylpropanamide (38)

The compound was prepared by general procedure i1 with Eix (70.5 mg, 0.37 mmol), 3-phenylpropanoic acid (55.6 mg, 0.37 mmol), EDCI (92 mg, 0.482 mmol), HOBt (55.1 mg, 0.408 mmol) and DIPEA (92 μL, 0.556 mmol) in CH₂Cl₂ (5 mL), stirred at room temperature for 17 h. After workup, the crude was purified by Isco Combi-Flash Companion column chromatography (0-5% methanol in CH₂Cl₂) to give 38 (86.7 mg, 73%). ¹H NMR (400 MHz, d₆-DMSO) δ 7.76 (br t, 1H), 7.30-7.10 (m, 5H), 7.03-6.93 (m, 2H), 6.85 (d, 1H), 3.70 (d, 1H), 3.49 (d, 1H), 3.34 (dt, 1H), 3.07 (dt, 1H), 2.81 (t, 2H), 2.69-2.59 (m, 1H), 2.56-2.51 (m overlapped with d₆-DMSO, 1H), 2.45-2.36 (m, 3H), 2.28 (s, 3H), 2.13 (s, 3H); LC-MS (ESI) m/z 323.2 [M+H]⁺.

### Example 39: 3-Cyclohexyl-N-(((3S)-2,5-dimethyl-1,2,3,4-tetrahydroisoquinolin-3-yl)methyl)propanamide (39)

The compound was prepared by general procedure i1 with Eix (78.8 mg, 0.414 mmol), 3-cyclohexylpropanoic acid (44.0 μL, 0.414 mmol), EDCI (103 mg, 0.538 mmol), HOBt (61.6 mg, 0.456 mmol) and DIPEA (103 μL, 0.621 mmol) in CH₂Cl₂ (5 mL), stirred at room temperature for 17 h. After workup, the crude was purified by Isco Combi-Flash Companion column chromatography (0-5% methanol in CH₂Cl₂) to give 39 (0.1 g, 74%). ¹H NMR (400 MHz, d₆-DMSO) δ 7.72 (br t, 1H), 7.07-6.95 (m, 2H), 6.86 (d, 1H), 3.73 (d, 1H), 3.51 (d, 1H), 3.41-3.31 (m overlapped with H₂O, 1H), 3.09 (dt, 1H), 2.71-2.55 (m, 2H), 2.49-2.41 (m, 1H), 2.32 (s, 3H), 2.16 (s, 3H), 2.10 (t, 2H), 1.76-1.57 (m, 5H), 1.40 (q, 2H), 1.27-1.06 (m, 4H), 0.94-0.79 (m, 2H); LC-MS (ESI) m/z 329.3 [M+H]⁺.

### Example 40: N-[(5-Chloro-2-methyl-1,2,3,4-tetrahydroisoquinolin-3-yl)methyl]-1,2,3,4-tetrahydronaphthalene-2-carboxamide (40)

The compound was prepared by general procedure i1 with Fix (20.5 mg, 0.097 mmol), 1,2,3,4-tetrahydro-2-naphthoic acid (16.3 mg, 0.092 mmol), 1-(3-dimethylaminopropyl)-3-ethylcarbodiimide hydrochloride (28 mg, 0.146 mmol), 1-hydroxybenzotriazole hydrate (19.7 mg, 0.146 mmol) and N,N-diisopropylethylamine (59.3 μL, 0.341 mmol) in dichloromethane (1 mL), stirred at room temperature for 16 h. After workup, the crude was purified by flash column chromatography (SiO₂, dichloromethane/methanol=98/2) to give 40 as a yellow solid (23.6 mg, 66%). ¹H NMR (CDCl₃, 400 MHz) δ 7.23 (d, 1H), 7.13-7.04 (m, 5H), 6.95 (d, 1H), 6.27 (br s, 1H), 3.83 (dd, 1H), 3.70 (d, 1H), 3.54 (dt, 1H), 3.45-3.36 (m, 1H), 3.05 (dd, 1H), 2.98-2.78 (m, 5H), 2.69-2.52 (m, 2H), 2.37 (s, 3H), 2.17-2.07 (m, overlapped with H₂O, 1H), 1.98-1.86 (m, 1H); LC-MS (ESI) m/z 369.2 [M+H]⁺.

### Example 41: N-[(5-Chloro-2-methyl-1,2,3,4-tetrahydroisoquinolin-3-yl)methyl]-2,3-dihydro-1H-indene-2-carboxamide (41)

The compound was prepared by general procedure i1 with Fix (59 mg, 0.28 mmol), 2-indanecarboxylic acid (43.1 mg, 0.266 mmol), 1-(3-dimethylaminopropyl)-3-ethylcarbodiimide hydrochloride (80.5 mg, 0.42 mmol), 1-hydroxybenzotriazole hydrate (56.8 mg, 0.42 mmol) and N,N-diisopropylethylamine (0.171 mL, 0.98 mmol) in dichloromethane (2.8 mL), stirred at room temperature for 16 h. After workup, the crude was purified by flash column chromatography (SiO₂, dichloromethane/methanol=98/2) to give 41 as a brown solid (67 mg, 67%). ¹H NMR (CDCl₃, 400 MHz) δ 7.25-7.07 (m, 6H), 6.95 (d, 1H), 6.29 (br s, 1H), 3.83 (d, 1H), 3.70 (d, 1H), 3.52 (dt, 1H), 3.41 (ddd, 1H), 3.29-3.12 (m, 5H), 2.94-2.78 (m, 2H), 2.63 (dd, 1H), 2.36 (s, 3H); LC-MS (ESI) m/z 355.2 [M+H]⁺.

### Example 42: N-{[(3R)-5-Chloro-2-methyl-1,2,3,4-tetrahydroisoquinolin-3-yl]methyl}-1,2,3,4-tetrahydronaphthalene-2-carboxamide (42)

The compound was prepared by general procedure i1 with Gix (44.8 mg, 0.213 mmol), 1,2,3,4-tetrahydro-2-naphthoic acid (35.6 mg, 0.202 mmol), 1-(3-dimethylaminopropyl)-3-ethylcarbodiimide hydrochloride (61.1 mg, 0.319 mmol), 1-hydroxybenzotriazole hydrate (43.1 mg, 0.319 mmol) and N,N-diisopropylethylamine (0.13 mL, 0.744 mmol) in dichloromethane (2.1 mL), stirred at room temperature for 16 h. After workup, the crude was purified by flash column chromatography (SiO₂, dichloromethane/methanol=98/2) to give 42 as a yellow solid (45.4 mg, 61%). ¹H NMR (CDCl₃, 400 MHz) δ 7.23 (d, 1H), 7.13-7.04 (m, 5H), 6.95 (d, 1H), 6.25 (br s, 1H), 3.84 (dd, 1H), 3.70 (d, 1H), 3.55 (dt, 1H), 3.45-3.36 (m, 1H), 3.05 (dd, 1H), 2.98-2.77 (m, 5H), 2.69-2.52 (m, 2H), 2.37 (s, 3H), 2.15-2.06 (m, overlapped with H₂O, 1H), 1.98-1.86 (m, 1H); LC-MS (ESI) m/z 369.2 [M+H]⁺.

### Example 43: N-{[(3R)-5-Chloro-2-methyl-1,2,3,4-tetrahydroisoquinolin-3-yl]methyl}-2,3-dihydro-1H-indene-2-carboxamide (43)

The compound was prepared by general procedure i1 with Gix (44.8 mg, 0.213 mmol), 2-indanecarboxylic acid (32.8 mg, 0.202 mmol), 1-(3-dimethylaminopropyl)-3-ethylcarbodiimide hydrochloride (61.1 mg, 0.319 mmol), 1-hydroxybenzotriazole hydrate (43.1 mg, 0.319 mmol) and N,N-diisopropylethylamine (0.13 mL, 0.744 mmol) in dichloromethane (2.1 mL), stirred at room temperature for 17 h. After workup, the crude was purified by flash column chromatography (SiO₂, dichloromethane/methanol=98/2) to give 43 as a brown solid (39.8 mg, 53%). ¹H NMR (CDCl₃, 400 MHz) δ 7.25-7.07 (m, 6H), 6.95 (d, 1H), 6.24 (br s, 1H), 3.84 (d, 1H), 3.71 (d, 1H), 3.54 (dt, 1H), 3.41 (ddd, 1H), 3.30-3.12 (m, 5H), 2.95-2.78 (m, 2H), 2.64 (dd, 1H), 2.37 (s, 3H); LC-MS (ESI) m/z 355.2 [M+H]⁺.

### Example 44: N-((5-Bromo-2-methyl-1,2,3,4-tetrahydroisoquinolin-3-yl)methyl)-3-phenylpropanamide (44)

The compound was prepared by general procedure i1 with Hix (51 mg, 0.2 mmol), 3-phenylpropanoic acid (30 mg, 0.200 mmol), EDCI (49.7 mg, 0.26 mmol), HOBt (30 mg, 0.22 mmol) and DIPEA (50 μL, 0.3 mmol) in CH₂Cl₂ (5 mL), stirred at room temperature for 17 h. After workup, the crude was purified by Isco Combi-Flash Companion column chromatography (0-3% methanol in CH₂Cl₂) to give 44 (54.6 mg, 71%). ¹H NMR (600 MHz, d₆-DMSO) δ 7.86 (br t, 1H), 7.43 (t, 1H), 7.28-7.17 (m, 4H), 7.14 (t, 1H), 7.11-7.06 (m, 2H), 3.75 (d, 1H), 3.53 (d, 1H), 3.36-3.27 (m overlapped with H₂O, 1H), 3.11 (dt, 1H), 2.80 (t, 2H), 2.70-2.60 (m, 2H), 2.54-2.46 (m overlapped with do-DMSO, 1H), 2.40 (t, 2H), 2.27 (s, 3H); LC-MS (ESI) m/z 388.1 [M+H]⁺.

### Example 45: N-((5-Bromo-2-methyl-1,2,3,4-tetrahydroisoquinolin-3-yl)methyl)-3-(3-fluorophenyl)propanamide (45)

The compound was prepared by general procedure i1 with Hix (46 mg, 0.18 mmol), 3-(3-fluorophenyl) propanoic acid (30 mg, 0.18 mmol), EDCI (44.8 mg, 0.234 mmol), HOBt (26.8 mg, 0.198 mmol) and DIPEA (45.0 μL, 0.27 mmol) in CH₂Cl₂ (5 mL), stirred at room temperature for 17 h. After workup, the crude was purified by Isco Combi-Flash Companion column chromatography (0-3% methanol in CH₂Cl₂) to give 45 (55.4 mg, 76%). ¹H NMR (400 MHz, d₆-DMSO) δ 7.87 (t, 1H), 7.45-7.38 (m, 1H), 7.32-7.23 (m, 1H), 7.12-7.00 (m, 4H), 6.95 (t, 1H), 3.75 (d, 1H), 3.52 (d, 1H), 3.37-3.27 (m overlapped with H₂O, 1H), 3.17-3.05 (m, 1H), 2.83 (t, 2H), 2.70-2.57 (m, 2H), 2.52-2.45 (m overlapped with d₆-DMSO, 1H), 2.42 (t, 2H), 2.27 (s, 3H); LC-MS (ESI) m/z 406.1 [M+H]⁺.

### Example 46: N-((5-Bromo-2-methyl-1,2,3,4-tetrahydroisoquinolin-3-yl)methyl)-3-cyclohexylpropanamide (46)

The compound was prepared by general procedure i1 with Hix (52 mg, 0.204 mmol), 3-cyclohexylpropanoic acid (22 μL, 0.204 mmol), EDCI (50.6 mg, 0.265 mmol), HOBt (30.3 mg, 0.224 mmol) and DIPEA (50 μL, 0.306 mmol) in CH₂Cl₂ (5 mL), stirred at room temperature for 17 h. After workup, the crude was purified by Isco Combi-Flash Companion column chromatography (0-3% methanol in CH₂Cl₂) to give 46 (72.4 mg, 90%). ¹H NMR (400 MHz, d₆-DMSO) δ 7.82 (br t, 1H), 7.44 (dd, 1H), 7.15-7.02 (m, 2H), 3.78 (d, 1H), 3.56 (d, 1H), 3.45-3.24 (m overlapped with H₂O, 1H), 3.13 (dt, 1H), 2.75-2.59 (m, 2H), 2.56-2.50 (m overlapped with do-DMSO, 1H), 2.31 (s, 3H), 2.10 (t, 2H), 1.75-1.57 (m, 5H), 1.38 (q, 2H), 1.27-1.06 (m, 4H), 0.91-0.78 (m, 2H); LC-MS (ESI) m/z 394.1 [M+H]⁺.

### Example 47: N-((5-Bromo-2-methyl-1,2,3,4-tetrahydroisoquinolin-3-yl)methyl)-2,3-dihydro-1H-indene-2-carboxamide (47)

The compound was prepared by general procedure i1 with Hix (51 mg, 0.2 mmol), 2,3-dihydro-1H-indene-2-carboxylic acid (32 mg, 0.2 mmol), EDCI (49.7 mg, 0.26 mmol), HOBt (29.7 mg, 0.22 mmol) and DIPEA (50 μL, 0.3 mmol) in CH₂Cl₂ (5 mL), stirred at room temperature for 17 h. After workup, the crude was purified by Isco Combi-Flash Companion column chromatography (0-3% methanol in CH₂Cl₂) to give 47 (58.8 mg, 74%). ¹H NMR (400 MHz, d₆-DMSO) δ 8.00 (br t, 1H), 7.47-7.40 (m, 1H), 7.22-7.14 (m, 2H), 7.14-7.05 (m, 4H), 3.79 (d, 1H), 3.56 (d, 1H), 3.37 (dt, 1H), 3.27-3.15 (m, 2H), 3.05 (d, 2H), 3.03 (d, 2H), 2.82-2.65 (m, 2H), 2.62-2.50 (m overlapped with do-DMSO, 1H), 2.33 (s, 3H); LC-MS (ESI) m/z 400.1 [M+H]⁺.

### Example 48: N-((5-Bromo-2-methyl-1,2,3,4-tetrahydroisoquinolin-3-yl)methyl)-3,4-dichlorobenzamide (48)

The compound was prepared by general procedure i2 with Hix (54 mg, 0.212 mmol), 3,4-dichlorobenzoyl chloride (44 g, 0.212 mmol) and triethylamine (35 μL, 0.254 mmol) in CH₂C₁₂ (5 mL), stirred at room temperature for 17 h. After workup, the crude was purified by Isco Combi-Flash Companion column chromatography (0-3% methanol in CH₂Cl₂) to give 48 (76.6 mg, 85%). ¹H NMR (400 MHz, d₆-DMSO) δ 8.69 (t, 1H), 8.06 (d, 1H), 7.81 (dd, 1H), 7.76 (d, 1H), 7.43 (dd, 1H), 7.14-7.05 (m, 2H), 3.80 (d, 1H), 3.61 (d, 1H), 3.55 (dt, 1H), 3.31-3.22 (m, 1H), 3.01-2.91 (m, 1H), 2.73 (dd, 1H), 2.58 (dd, 1H), 2.37 (s, 3H); LC-MS (ESI) m/z 429.0 [M+H]⁺.

### Example 49: N-((5-Bromo-2-methyl-1,2,3,4-tetrahydroisoquinolin-3-yl)methyl)-4-ethylbenzamide (49)

The compound was prepared by general procedure i1 with Hix (47 mg, 0.184 mmol), 4-ethylbenzoic acid (27.7 mg, 0.184 mmol), EDCI (45.8 mg, 0.239 mmol), HOBt (27.4 mg, 0.203 mmol) and DIPEA (50 μL, 0.276 mmol) in CH₂Cl₂ (5 mL), stirred at room temperature for 17 h. After workup, the crude was purified by Isco Combi-Flash Companion column chromatography (0-3% methanol in CH₂Cl₂) to give 49 (63.6 mg, 89%). ¹H NMR (400 MHz, d₆-DMSO) δ 8.40 (br t, 1H), 7.75 (d, 2H), 7.43 (d, 1H), 7.29 (d, 2H), 7.14-7.03 (m, 2H), 3.80 (d, 1H), 3.59 (d, 1H), 3.54 (dt, 1H), 3.26 (dt, 1H), 3.01-2.91 (m, 1H), 2.76 (dd, 1H), 2.64 (q, 2H), 2.57 (dd, 1H), 2.38 (s, 3H), 1.18 (t, 3H); LC-MS (ESI) m/z 388.1 [M+H]⁺.

### Example 50: N-(((3R)-5-Bromo-2-methyl-1,2,3,4-tetrahydroisoquinolin-3-yl)methyl)-3-phenylpropanamide (50)

The compound was prepared by general procedure i1 with Iix (61 mg, 0.239 mmol), 3-phenylpropanoic acid (35.9 mg, 0.239 mmol), EDCI (59.4 mg, 0.311 mmol), HOBt (35.5 mg, 0.263 mmol) and DIPEA (60.0 μL, 0.359 mmol) in CH₂Cl₂ (5 mL), stirred at room temperature for 17 h. After workup, the crude was purified by Isco Combi-Flash Companion column chromatography (0-5% methanol in CH₂Cl₂) to give 50 (76.7 mg, 83%). ¹H NMR (400 MHz, d₆-DMSO) δ 7.86 (br t, 1H), 7.43 (t, 1H), 7.28-7.17 (m, 4H), 7.16-7.11 (m, 1H), 7.11-7.05 (m, 2H), 3.76 (d, 1H), 3.52 (d, 1H), 3.38-3.27 (m overlapped with H₂O, 1H), 3.17-3.06 (m, 1H), 2.80 (t, 2H), 2.70-2.58 (m, 2H), 2.53-2.45 (m overlapped with do-DMSO, 1H), 2.40 (t, 2H), 2.27 (s, 3H); LC-MS (ESI) m/z 388.1 [M+H]⁺.

### Example 51: N-(((3R)-5-Bromo-2-methyl-1,2,3,4-tetrahydroisoquinolin-3-yl)methyl)-3-(3-fluorophenyl)propanamide (51)

The compound was prepared by general procedure i1 with Iix (55 mg, 0.216 mmol), 3-(3-fluorophenyl) propanoic acid (36.2 mg, 0.216 mmol), EDCI (53.5 mg, 0.28 mmol), HOBt (32 mg, 0.237 mmol) and DIPEA (50 μL, 0.323 mmol) in CH₂Cl₂ (5 mL), stirred at room temperature for 17 h. After workup, the crude was purified by Isco Combi-Flash Companion column chromatography (0-5% methanol in CH₂Cl₂) to give 51 (62.9 mg, 72%). ¹H NMR (400 MHz, d₆-DMSO) δ 7.87 (br t, 1H), 7.47-7.39 (m, 1H), 7.33-7.23 (m, 1H), 7.13-7.00 (m, 4H), 7.00-6.91 (m, 1H), 3.75 (d, 1H), 3.52 (d, 1H), 3.36-3.28 (m overlapped with H₂O, 1H), 3.18-3.04 (m, 1H), 2.83 (t, 2H), 2.71-2.56 (m, 2H), 2.53-2.45 (m overlapped with do-DMSO, 1H), 2.43 (t, 2H), 2.27 (s, 3H); LC-MS (ESI) m/z 406.0 [M+H]⁺.

### Example 52: N-(((3R)-5-Bromo-2-methyl-1,2,3,4-tetrahydroisoquinolin-3-yl)methyl)-3-cyclohexylpropanamide (52)

The compound was prepared by general procedure i1 with Iix (55 mg, 0.216 mmol), 3-cyclohexylpropanoic acid (23 μL, 0.216 mmol), EDCI (53.5 mg, 0.28 mmol), HOBt (32 mg, 0.237 mmol) and DIPEA (50 μL, 0.323 mmol) in CH₂Cl₂ (5 mL), stirred at room temperature for 17 h. After workup, the crude was purified by Isco Combi-Flash Companion column chromatography (0-5% methanol in CH₂Cl₂) to give 52 (31.3 mg, 37%). ¹H NMR (400 MHz, d₆-DMSO) δ 7.81 (br t, 1H), 7.43 (dd, 1H), 7.11-7.04 (m, 2H), 3.77 (d, 1H), 3.53 (d, 1H), 3.38-3.26 (m overlapped with H₂O, 1H), 3.12 (dt, 1H), 2.75-2.58 (m, 2H), 2.56-2.47 (m overlapped with do-DMSO, 1H), 2.30 (s, 3H), 2.09 (t, 2H), 1.75-1.54 (m, 5H), 1.38 (q, 2H), 1.24-1.04 (m, 4H), 0.91-0.72 (m, 2H); LC-MS (ESI) m/z 394.1 [M+H]⁺.

### Example 53: N-(((3R)-5-Bromo-2-methyl-1,2,3,4-tetrahydroisoquinolin-3-yl)methyl)-2,3-dihydro-1H-indene-2-carboxamide (53)

The compound was prepared by general procedure i1 with Iix (120 mg, 0.47 mmol), 2,3-dihydro-1H-indene-2-carboxylic acid (76.3 mg, 0.47 mmol), EDCI (117 mg, 0.611 mmol), HOBt (70 mg, 0.517 mmol) and DIPEA (0.117 mL, 0.705 mmol) in CH₂Cl₂ (5 mL), stirred at room temperature for 17 h. After workup, the crude was purified by Isco Combi-Flash Companion column chromatography (0-5% methanol in CH₂Cl₂) to give 53 (150 mg, 80%). ¹H NMR (400 MHz, d₆-DMSO) δ 8.00 (br t, 1H), 7.48-7.40 (m, 1H), 7.23-7.14 (m, 2H), 7.13-7.05 (m, 4H), 3.79 (d, 1H), 3.56 (d, 1H), 3.43-3.30 (m overlapped with H₂O, 1H), 3.28-3.13 (m, 2H), 3.09-2.99 (m, 4H), 2.82-2.63 (m, 2H), 2.61-2.50 (m, 1H), 2.33 (s, 3H); LC-MS (ESI) m/z 400.1 [M+H]⁺.

### Example 54: N-(((3R)-5-Bromo-2-methyl-1,2,3,4-tetrahydroisoquinolin-3-yl)methyl)-3,4-dichlorobenzamide (54)

The compound was prepared by general procedure i2 with Iix (120 mg, 0.47 mmol), 3,4-dichlorobenzoyl chloride (98.5 mg, 0.47 mmol) and triethylamine (79 μL, 0.564 mmol) in CH₂C₁₂ (5 mL), stirred at room temperature for 17 h. After workup, the crude was purified by Isco Combi-Flash Companion column chromatography (0-5% methanol in CH₂Cl₂) to give 54 (170 mg, 84%). ¹H NMR (400 MHz, de-DMSO) δ 8.69 (br t, 1H), 8.06 (d, 1H), 7.81 (dd, 1H), 7.76 (d, 1H), 7.43 (dd, 1H), 7.13-7.04 (m, 2H), 3.80 (d, 1H), 3.61 (d, 1H), 3.55 (dt, 1H), 3.32-3.21 (m overlapped with H₂O, 1H), 3.00-2.88 (m, 1H), 2.73 (dd, 1H), 2.58 (dd, 1H), 2.37 (s, 3H); LC-MS (ESI) m/z 429.0 [M+H]⁺.

### Example 55: N-(((3R)-5-Bromo-2-methyl-1,2,3,4-tetrahydroisoquinolin-3-yl)methyl)-4-ethylbenzamide (55)

The compound was prepared by general procedure i1 with Iix (120 mg, 0.47 mmol), 4-ethylbenzoic acid (70.6 mg, 0.47 mmol), EDCI (117 mg, 0.611 mmol), HOBt (70 mg, 0.517 mmol) and DIPEA (0.117 mL, 0.705 mmol) in CH₂Cl₂ (5 mL), stirred at room temperature for 17 h. After workup, the crude was purified by Isco Combi-Flash Companion column chromatography (0-5% methanol in CH₂Cl₂) to give 55 (150 mg, 82%). ¹H NMR (400 MHz, d₆-DMSO) δ 8.40 (br t), 7.75 (d, 2H), 7.43 (d, 1H), 7.29 (d, 2H), 7.13-7.04 (m, 2H), 3.80 (d, 1H), 3.60 (d, 1H), 3.54 (dt, 1H), 3.31-3.21 (m, 1H), 3.00-2.89 (m, 1H), 2.79-2.52 (m, 4H), 2.38 (s, 3H), 1.18 (t, 3H); LC-MS (ESI) m/z 388.1 [M+H]⁺.

### Example 56: N-(((3S)-5-Bromo-2-methyl-1,2,3,4-tetrahydroisoquinolin-3-yl)methyl)-3-phenylpropanamide (56)

The compound was prepared by general procedure i1 with Jix (90 mg, 0.353 mmol), 3-phenylpropanoic acid (53 mg, 0.353 mmol), EDCI (87.6 mg, 0.459 mmol), HOBt (52.4 mg, 0.388 mmol) and DIPEA (87 μL, 0.529 mmol) in CH₂Cl₂ (5 mL), stirred at room temperature for 17 h. After workup, the crude was purified by Isco Combi-Flash Companion column chromatography (0-5% methanol in CH₂Cl₂) to give 56 (0.1 g, 73%). ¹H NMR (400 MHz, d₆-DMSO) δ 7.86 (br t, 1H), 7.46-7.39 (m, 1H), 7.29-7.04 (m, 7H), 3.76 (d, 1H), 3.52 (d, 1H), 3.37-3.26 (m overlapped with H₂O, 1H), 3.12 (dt, 1H), 2.80 (t, 2H), 2.72-2.57 (m, 2H), 2.55-2.45 (m overlapped with do-DMSO, 1H), 2.40 (t, 2H), 2.27 (s, 3H); LC-MS (ESI) m/z 388.1 [M+H]⁺.

### Example 57: N-(((3S)-5-Bromo-2-methyl-1,2,3,4-tetrahydroisoquinolin-3-yl)methyl)-3-(3-fluorophenyl)propanamide (57)

The compound was prepared by general procedure i1 with Jix (86 mg, 0.337 mmol), 3-(3-fluorophenyl) propanoic acid (56.7 mg, 0.337 mmol), EDCI (83.7 mg, 0.438 mmol), HOBt (50.1 mg, 0.371 mmol) and DIPEA (84 μL, 0.506 mmol) in CH₂Cl₂ (5 mL), stirred at room temperature for 17 h. After workup, the crude was purified by Isco Combi-Flash Companion column chromatography (0-5% methanol in CH₂Cl₂) to give 57 (98 mg, 72%). ¹H NMR (400 MHz, d₆-DMSO) δ 7.87 (br t, 1H), 7.46-7.38 (m, 1H), 7.33-7.22 (m, 1H), 7.13-7.00 (m, 4H), 7.00-6.92 (m, 1H), 3.75 (d, 1H), 3.52 (d, 1H), 3.39-3.26 (m overlapped with H₂O, 1H), 3.18-3.04 (m, 1H), 2.83 (t, 2H), 2.71-2.58 (m, 2H), 2.52-2.45 (m overlapped with de-DMSO, 1H), 2.42 (t, 2H), 2.27 (s, 3H); LC-MS (ESI) m/z 406.1 [M+H]⁺.

### Example 58: N-(((3S)-5-Bromo-2-methyl-1,2,3,4-tetrahydroisoquinolin-3-yl)methyl)-3-cyclohexylpropanamide (58)

The compound was prepared by general procedure i1 with Jix (83 mg, 0.325 mmol), 3-cyclohexylpropanoic acid (35 μL, 0.325 mmol), EDCI (80.8 mg, 0.423 mmol), HOBt (48.3 mg, 0.358 mmol) and DIPEA (81 μL, 0.488 mmol) in CH₂Cl₂ (5 mL), stirred at room temperature for 17 h. After workup, the crude was purified by Isco Combi-Flash Companion column chromatography (0-5% methanol in CH₂Cl₂) to give 58 (33 mg, 26%). ¹H NMR (400 MHz, d₆-DMSO) δ 7.81 (br t, 1H), 7.43 (t, 1H), 7.13-7.03 (m, 2H), 3.77 (d, 1H), 3.53 (d, 1H), 3.37-3.26 (m overlapped with H₂O, 1H), 3.20-3.06 (m, 1H), 2.75-2.60 (m, 2H), 2.57-2.49 (m overlapped with d₆-DMSO, 1H), 2.30 (s, 3H), 2.09 (t, 2H), 1.74-1.53 (m, 5H), 1.38 (q, 2H), 1.24-1.01 (m, 4H), 0.92-0.74 (m, 2H); LC-MS (ESI) m/z 394.1 [M+H]⁺.

### Example 59: N-(((3S)-5-Bromo-2-methyl-1,2,3,4-tetrahydroisoquinolin-3-yl)methyl)-2,3-dihydro-1H-indene-2-carboxamide (59)

The compound was prepared by general procedure i1 with ((3S)-5-bromo-2-methyl-1,2,3,4-tetrahydroisoquinolin-3-yl)methanamine Jix (90 mg, 0.353 mmol), 2,3-dihydro-1H-indene-2-carboxylic acid (57.2 mg, 0.353 mmol), EDCI (87.6 mg, 0.459 mmol), HOBt (52.4 mg, 0.388 mmol) and DIPEA (87 μL, 0.529 mmol) in CH₂Cl₂ (5 mL), stirred at room temperature for 17 h. After workup, the crude was purified by Isco Combi-Flash Companion column chromatography (0-5% methanol in CH₂Cl₂) to give 59 (110 mg, 78%). ¹H NMR (400 MHz, d₆-DMSO) δ 8.00 (br t, 1H), 7.48-7.39 (m, 1H), 7.21-7.14 (m, 2H), 7.13-7.05 (m, 4H), 3.79 (d, 1H), 3.56 (d, 1H), 3.37 (dt, 1H), 3.28-3.15 (m, 2H), 3.07-2.99 (m, 4H), 2.82-2.62 (m, 2H), 2.54 (dd, 1H), 2.33 (s, 3H); LC-MS (ESI) m/z 400.1 [M+H]⁺.

### Example 60: N-(((3S)-5-Bromo-2-methyl-1,2,3,4-tetrahydroisoquinolin-3-yl)methyl)-3,4-dichlorobenzamide (60)

The compound was prepared by general procedure i2 with Jix (75 mg, 0.294 mmol), 3,4-dichlorobenzoyl chloride (61.6 mg, 0.294 mmol) and triethylamine (50 μL, 0.353 mmol) in CH₂Cl₂ (5 mL), stirred at room temperature for 17 h. After workup, the crude was purified by Isco Combi-Flash Companion column chromatography (0-5% methanol in CH₂Cl₂) to give 60 (0.106 g, 84%). ¹H NMR (400 MHz, d₆-DMSO) δ 8.69 (t, 1H), 8.06 (d, 1H), 7.81 (dd, 1H), 7.76 (d, 1H), 7.47-7.39 (m, 1H), 7.14-7.04 (m, 2H), 3.80 (d, 1H), 3.61 (d, 1H), 3.55 (dt, 1H), 3.31-3.21 (m overlapped with H₂O, 1H), 3.02-2.90 (m, 1H), 2.73 (dd, 1H), 2.58 (dd, 1H), 2.37 (s, 3H); LC-MS (ESI) m/z 429.0 [M+H]⁺.

### Example 61: N-((2,6-Dimethyl-1,2,3,4-tetrahydroisoquinolin-3-yl)methyl)-4-(ethylsulfanyl)benzamide (61)

The compound was prepared by general procedure i1 with Kix (57 mg, 0.3 mmol), 4-(ethylthio)benzoic acid (65.6 mg, 0.36 mmol), 1-(3-dimethylaminopropyl)-3-ethylcarbodiimide hydrochloride (74.7 mg, 0.39 mmol), 1-hydroxybenzotriazole hydrate (52.7 mg, 0.39 mmol) and N,N-diisopropylethylamine (160 μL, 0.918 mmol) in dichloromethane (3 mL), stirred at room temperature for 17 h. After workup, the crude was purified by Isco Combi-Flash Companion column chromatography (0-6% methanol in CH₂Cl₂) to give 61 as an orange solid (67.2 mg, 63%). ¹H NMR (CDCl₃, 400 MHz) δ 7.80 (dt, 2H), 7.30 (dd, 2H), 7.08-6.93 (m, 3H), 4.04 (t, 1H), 3.95-3.74 (m, 2H), 3.59 (ddd, 1H), 3.23 (s, 1H), 3.05-2.89 (m, 4H), 2.89-2.79 (m, 1H), 2.58 (d, 4H), 2.30 (s, 3H), 1.34 (t, 3H); LC-MS (ESI) m/z 355.2 [M+H]⁺.

### Example 62: 3,4-Dichloro-N-((2,6-dimethyl-1,2,3,4-tetrahydroisoquinolin-3-yl)methyl)benzamide (62)

The compound was prepared by general procedure i1 with Kix (57 mg, 0.3 mmol), 3,4-dichlorobenzoic acid (68.7 mg, 0.36 mmol), 1-(3-dimethylaminopropyl)-3-ethylcarbodiimide hydrochloride (74.7 mg, 0.39 mmol), 1-hydroxybenzotriazole hydrate (52.7 mg, 0.39 mmol) and N,N-diisopropylethylamine (160 μL, 0.918 mmol) in dichloromethane (3 mL), stirred at room temperature for 17 h. After workup, the crude was purified by Isco Combi-Flash Companion column chromatography (0-6% methanol in CH₂Cl₂) to give 62 as an orange solid (57.1 mg, 52%). ¹H NMR (CDCl₃, 400 MHz) δ 7.99 (s, 1H), 7.77 (d, 1H), 7.54-7.47 (m, 1H), 7.11-6.95 (m, 3H), 4.10 (d, 1H), 3.97 (d, 1H), 3.86 (d, 1H), 3.69-3.54 (m, 1H), 3.33 (s, 1H), 3.07-2.95 (m, 1H), 2.91-2.76 (m, 1H), 2.67-2.57 (m, 3H), 2.30 (s, 3H); LC-MS (ESI) m/z 364.1 [M+H]⁺.

### Example 63: N-[(2,6-Dimethyl-1,2,3,4-tetrahydroisoquinolin-3-yl)methyl]-3-phenylpropanamide (63)

The compound was prepared by general procedure i1 with Kix (40 mg, 0.21 mmol), 3-phenylpropionic acid (30 mg, 0.2 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=98/2) to give 63 as a yellow solid (47.7 mg, 74%). ¹H NMR (CDCl₃, 400 MHz) δ 7.30-7.24 (m, overlapped with CDCl₃, 2H), 7.22-7.15 (m, 3H), 6.97-6.86 (m, 3H), 6.05 (br s, 1H), 3.76 (d, 1H), 3.62 (d, 1H), 3.42 (dt, 1H), 3.30 (ddd, 1H), 2.97 (t, 2H), 2.81-2.73 (m, 1H), 2.68-2.56 (m, 2H), 2.50 (t, 2H), 2.30 (s, 3H); LC-MS (ESI) m/z 323.2 [M+H]⁺.

### Example 64: 3-Cyclohexyl-N-[(2,6-dimethyl-1,2,3,4-tetrahydroisoquinolin-3-yl)methyl]propanamide (64)

The compound was prepared by general procedure i1 with Kix (40 mg, 0.21 mmol), 3-cyclohexylpropionic acid (34.2 μL, 0.2 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=98/2) to give 64 as a yellow solid (65.6 mg, quantitative yield). ¹H NMR (CDCl₃, 400 MHz) δ 6.97-6.88 (m, 3H), 6.64 (br s, 1H), 3.86 (d, 1H), 3.72 (d, 1H), 3.49 (dt, 1H), 3.37 (ddd, 1H), 2.98-2.90 (m, 1H), 2.81-2.68 (m, 2H), 2.40 (s, 3H), 2.30-2.22 (m, 4H), 2.21-2.14 (m, 2H), 1.74-1.57 (m, 4H), 1.55-1.45 (m, 2H), 1.27-1.05 (m, 4H), 0.94-0.82 (m, 2H); LC-MS (ESI) m/z 329.2 [M+H]⁺.

### Example 65: N-(((3R)-2,6-Dimethyl-1,2,3,4-tetrahydroisoquinolin-3-yl)methyl)-4-(trifluoromethyl)benzamide (65)

The compound was prepared by general procedure i1 with Lix (50 mg, 0.263 mmol), 4-(trifluoromethyl)benzoic acid (47.5 mg, 0.250 mmol), 1-(3-dimethylaminopropyl)-3-ethylcarbodiimide hydrochloride (75.6 mg, 0.395 mmol), 1-hydroxybenzotriazole hydrate (53.3 mg, 0.395 mmol) and N,N-diisopropylethylamine (160 μL, 0.921 mmol) in dichloromethane (5.3 mL), stirred at room temperature for 18 h. After workup, the crude was purified by flash column chromatography (3-5% methanol in dichloromethane) to give 65 as a white solid (65 mg, 72%). ¹H NMR (300 MHz, d₆-DMSO) δ 8.69 (t, 1H), 8.03 (d, 2H), 7.85 (d, 2H), 6.99-6.87 (m, 3H), 3.74 (d, 1H), 3.65-3.48 (m, 2H), 3.27-3.13 (m, 1H), 3.01-2.87 (m, 1H), 2.80 (dd, 1H), 2.65 (dd, 1H), 2.42 (s, 3H), 2.23 (s, 3H); LC-MS (ESI) m/z 363.2 [M+H]⁺.

### Example 66: N-(((3R)-2,6-Dimethyl-1,2,3,4-tetrahydroisoquinolin-3-yl)methyl)-4-(ethylsulfanyl)benzamide (66)

The compound was prepared by general procedure i1 with Lix (50 mg, 0.263 mmol), 4-(ethylthio)benzoic acid (45.5 mg, 0.25 mmol), 1-(3-dimethylaminopropyl)-3-ethylcarbodiimide hydrochloride (75.6 mg, 0.395 mmol), 1-hydroxybenzotriazole hydrate (53.3 mg, 0.395 mmol) and N,N-diisopropylethylamine (160 μL, 0.921 mmol) in dichloromethane (5.3 mL), stirred at room temperature for 18 h. After workup, the crude was purified by flash column chromatography (3-5% methanol in dichloromethane) to give 66 as a white solid (55 mg, 62%). ¹H NMR (300 MHz, d₆-DMSO) δ 8.41 (t, 1H), 7.78 (d, 2H), 7.35 (d, 2H), 6.99-6.85 (m, 3H), 3.75 (d, 1H), 3.65-3.44 (m, 2H), 3.24-3.11 (m, 1H), 3.05 (q, 2H), 2.98-2.85 (m, 1H), 2.79 (dd, 1H), 2.63 (dd, 1H), 2.42 (s, 3H), 2.23 (s, 3H), 1.26 (t, 3H); LC-MS (ESI) m/z 355.2 [M+H]⁺.

### Example 67: N-(((3R)-2,6-Dimethyl-1,2,3,4-tetrahydroisoquinolin-3-yl)methyl)-3-(3-fluorophenyl)propanamide (67)

The compound was prepared by general procedure i1 with Lix (73 mg, 0.384 mmol), 3-(3-fluorophenyl) propionic acid (61.3 mg, 0.364 mmol), 1-(3-dimethylaminopropyl)-3-ethylcarbodiimide hydrochloride (110 mg, 0.576 mmol), 1-hydroxybenzotriazole hydrate (78 mg, 0.576 mmol) and N,N-diisopropylethylamine (234 μL, 1.34 mmol) in dichloromethane (7.7 mL), stirred at room temperature for 18 h. After workup, the crude was purified by flash column chromatography (3% methanol in dichloromethane) to give 67 as a white solid (77 mg, 59%). ¹H NMR (300 MHz, d₆-DMSO) δ 7.81 (t, 1H), 7.29 (q, 1H), 7.09-6.94 (m, 3H), 6.94-6.79 (m, 3H), 3.70 (d, 1H), 3.50 (d, 1H), 3.29-3.18 (m overlapped with H₂O, 1H), 3.09-2.93 (m, 1H), 2.83 (t, 2H), 2.74-2.53 (m, 3H), 2.42 (t, 2H), 2.32 (s, 3H), 2.23 (s, 3H); LC-MS (ESI) m/z 341.2 [M+H]⁺.

### Example 68: N-(((3R)-2,6-Dimethyl-1,2,3,4-tetrahydroisoquinolin-3-yl)methyl)-3-phenylpropanamide (68)

The compound was prepared by general procedure i1 with Lix (50 mg, 0.263 mmol), 3-phenylpropionic acid (37.5 mg, 0.25 mmol), 1-(3-dimethylaminopropyl)-3-ethylcarbodiimide hydrochloride (75.6 mg, 0.395 mmol), 1-hydroxybenzotriazole hydrate (53.3 mg, 0.395 mmol) and N,N-diisopropylethylamine (160 μL, 0.921 mmol) in dichloromethane (5.3 mL), stirred at room temperature for 18 h. After workup, the crude was purified by flash column chromatography (3-5% methanol in dichloromethane) to give 68 as a white solid (46 mg, 58%). [α]_{D}²⁴=+39.01° (c=1, MeOH); ¹H NMR (300 MHz, d₆-DMSO) δ 7.80 (t, 1H), 7.30-7.12 (m, 5H), 6.95-6.81 (m, 3H), 3.68 (d, 1H), 3.48 (d, 1H), 3.31-3.20 (m overlapped with H₂O, 1H), 3.01 (dt, 1H), 2.81 (t, 2H), 2.69-2.53 (m, 3H), 2.40 (t, 2H), 2.31 (s, 3H), 2.23 (s, 3H); LC-MS (ESI) m/z 323.2 [M+H]⁺.

### Example 69: 3-(Cyclohexyl-N-(((3R)-2,6-dimethyl-1,2,3,4-tetrahydroisoquinolin-3-yl)methyl)propanamide (69)

The compound was prepared by general procedure i1 with Lix (50 mg, 0.263 mmol), 3-cyclohexylpropionic acid (39 μL, 0.25 mmol), 1-(3-dimethylaminopropyl)-3-ethylcarbodiimide hydrochloride (75.6 mg, 0.395 mmol), 1-hydroxybenzotriazole hydrate (53.3 mg, 0.395 mmol) and N,N-diisopropylethylamine (160 μL, 0.921 mmol) in dichloromethane (5.3 mL), stirred at room temperature for 18 h. After workup, the crude was purified by flash column chromatography (3-5% methanol in dichloromethane) to give 69 as a white solid (48 mg, 59%). ¹H NMR (300 MHz, d₆-DMSO) δ 7.76 (t, 1H), 6.95-6.83 (m, 3H), 3.69 (d, 1H), 3.48 (d, 1H), 3.31-3.20 (m overlapped with H₂O, 1H), 3.07-2.93 (m, 1H), 2.74-2.53 (m, 3H), 2.33 (s, 3H), 2.23 (s, 3H), 2.09 (t, 2H), 1.74-1.53 (m, 5H), 1.38 (q, 2H), 1.27-1.04 (m, 4H), 0.91-0.79 (m, 2H); LC-MS (ESI) m/z 329.3 [M+H]⁺.

### Example 70: N-{[(3R)-2,6-Dimethyl-1,2,3,4-tetrahydroisoquinolin-3-yl]methyl}-2,3-dihydro-1H-indene-2-carboxamide (70)

The compound was prepared by general procedure i1 with Lix (0.13 g, 0.683 mmol), 2-indanecarboxylic acid (0.111 g, 0.683 mmol), 1-(3-dimethylaminopropyl)-3-ethylcarbodiimide hydrochloride (0.196 g, 1.02 mmol), 1-hydroxybenzotriazole hydrate (0.139 g, 1.02 mmol) and N,N-diisopropylethylamine (0.298 mL, 1.71 mmol) in dichloromethane (6.80 mL), stirred at room temperature for 16 h. After workup, the crude was purified by flash column chromatography (SiO₂, dichloromethane/methanol=97/3) to give 70 as a white solid (0.137 g, 60%). [α]_{D}²⁴=+48.21° (c=1, MeOH); ¹H NMR (CDCl₃, 400 MHz) δ 7.23-7.13 (m, 4H), 6.99-6.90 (m, 3H), 6.24 (br s, 1H), 3.82 (d, 1H), 3.67 (d, 1H), 3.50 (dt, 1H), 3.39 (dt, 1H), 3.29-3.11 (m, 5H), 2.91-2.83 (m, 1H), 2.80-2.67 (m, 2H), 2.38 (s, 3H), 2.30 (s, 3H); LC-MS (ESI) m/z 335.2 [M+H]⁺.

### Example 71: N-{[(3R)-2,6-Dimethyl-1,2,3,4-tetrahydroisoquinolin-3-yl]methyl}-1,2,3,4-tetrahydronaphthalene-2-carboxamide (71)

The compound was prepared by general procedure i1 with Lix (40 mg, 0.21 mmol), 1,2,3,4-tetrahydro-2-naphthoic acid (37 mg, 0.21 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=98/2) to give 71 as a yellow liquid (49.9 mg, 68%). ¹H NMR (CDCl₃, 400 MHz) δ 7.18-7.10 (m, 4H), 6.98-6.90 (m, 3H), 6.29 (br s, 1H), 3.82 (dd, 1H), 3.68 (d, 1H), 3.51 (dt, 1H), 3.44-3.35 (m, 1H), 3.08-2.66 (m, 7H), 2.59-2.49 (m, 1H), 2.38 (s, 3H), 2.30 (s, 3H), 2.13-2.05 (m, 1H), 1.97-1.84 (m, 1H); LC-MS (ESI) m/z 349.2 [M+H]⁺.

### Example 72: N-{[(3R)-2,6-Dimethyl-1,2,3,4-tetrahydroisoquinolin-3-yl]methyl}-9H-fluorene-9-carboxamide (72)

The compound was prepared by general procedure i1 with Lix (40 mg, 0.21 mmol), fluorene-9-carboxylic acid (44.2 mg, 0.21 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=98/2) to give 72 as a yellow solid (50.1 mg, 62%). ¹H NMR (CDCl₃, 400 MHz) δ 7.78 (d, 2H), 7.71 (d, 1H), 7.64 (d, 1H), 7.44 (t, 2H), 7.38-7.31 (m, 2H), 6.93 (d, 1H), 6.86 (d, 1H), 6.82 (s, 1H), 5.91 (br s, 1H), 4.80 (s, 1H), 3.65 (d, 1H), 3.53 (d, 1H), 3.37-3.23 (m, 2H), 2.77-2.68 (m, 1H), 2.64 (dd, 1H), 2.49 (dd, 1H), 2.28 (s, 3H), 2.22 (s, 3H); LC-MS (ESI) m/z 383.2 [M+H]⁺.

### Example 73: 5-Bromo-N-{[(3R)-2,6-dimethyl-1,2,3,4-tetrahydroisoquinolin-3-yl]methyl}-2,3-dihydro-1H-indene-2-carboxamide (73)

The compound was prepared by general procedure i1 with Lix (40 mg, 0.21 mmol), 5-bromo-2,3-dihydro-1H-indene-2-carboxylic acid (50.7 mg, 0.21 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=97/3) to give 73 as a yellow solid (53.3 mg, 61%). ¹H NMR (CDCl₃, 400 MHz) δ 7.31 (s, 1H), 7.26 (d, overlapped with CDCl₃, 1H), 7.04 (d, 1H), 6.99-6.89 (m, 3H), 6.27 (br s, 1H), 3.81 (d, 1H), 3.68 (d, 1H), 3.49 (dt, 1H), 3.37 (ddd, 1H), 3.27-3.02 (m, 5H), 2.91-2.83 (m, 1H), 2.79-2.65 (m, 2H), 2.38 (s, 3H), 2.29 (s, 3H); LC-MS (ESI) m/z 415.1 [M+H]⁺.

### Example 74: N-{[(3R)-2,6-Dimethyl-1,2,3,4-tetrahydroisoquinolin-3-yl]methyl}-5-fluoro-2,3-dihydro-1H-indene-2-carboxamide (74)

The compound was prepared by general procedure i1 with Lix (40 mg, 0.21 mmol), 5-fluoro-2,3-dihydro-1H-indene-2-carboxylic acid (37.9 mg, 0.21 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=97/3) to give 74 as a yellow solid (51.6 mg, 70%). ¹H NMR (CDCl₃, 400 MHz) δ 7.10 (t, 1H), 6.97-6.79 (m, 5H), 6.31 (br s, 1H), 3.82 (d, 1H), 3.68 (d, 1H), 3.50 (dt, 1H), 3.38 (dt, 1H), 3.28-3.05 (m, 5H), 2.91-2.84 (m, 1H), 2.80-2.66 (m, 2H), 2.38 (s, 3H), 2.29 (s, 3H); LC-MS (ESI) m/z 353.2 [M+H]⁺.

### Example 75: N-{[(3R)-2,6-Dimethyl-1,2,3,4-tetrahydroisoquinolin-3-yl]methyl}-1H-indene-2-carboxamide (75)

The compound was prepared by general procedure i1 with Lix (50 mg, 0.263 mmol), 1H-indene-2-carboxylic acid (42.1 mg, 0.263 mmol), 1-(3-dimethylaminopropyl)-3-ethylcarbodiimide hydrochloride (75.6 mg, 0.394 mmol), 1-hydroxybenzotriazole hydrate (53.3 mg, 0.394 mmol) and N,N-diisopropylethylamine (0.114 mL, 0.657 mmol) in dichloromethane (2.6 mL), stirred at room temperature for 16 h. After workup, the crude was purified by flash column chromatography (SiO₂, dichloromethane/methanol=96/4) to give 75 as a brown solid (66.1 mg, 76%). ¹H NMR (CDCl₃, 400 MHz) δ 7.50-7.42 (m, 3H), 7.34-7.27 (m, 2H), 6.99-6.90 (m, 3H), 6.62 (br s, 1H), 3.86 (d, 1H), 3.72 (d, 1H), 3.67-3.60 (m, 3H), 3.50 (ddd, 1H), 3.01-2.94 (m, 1H), 2.84-2.73 (m, 2H), 2.44 (s, 3H), 2.27 (s, 3H); LC-MS (ESI) m/z 333.2 [M+H]⁺.

### Example 76: 1-(4-Chlorophenyl)-N-{[(3R)-2,6-dimethyl-1,2,3,4-tetrahydroisoquinolin-3-yl]methyl}cyclopentanecarboxamide (76)

The compound was prepared by general procedure i1 with Lix (40 mg, 0.21 mmol), 1-(4-chlorophenyl)-1-cyclopentanecarboxylic acid (47.2 mg, 0.21 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=98/2) to give 76 as a yellow liquid (45.9 mg, 55%). ¹H NMR (CDCl₃, 400 MHz) δ 7.31-7.25 (m, overlapped with CDCl₃, 4H), 6.94 (d, 1H), 6.88 (d, 1H), 6.83 (s, 1H), 5.91 (br s, 1H), 3.69 (d, 1H), 3.54 (d, 1H), 3.33-3.21 (m, 2H), 2.74-2.67 (m, 1H), 2.61 (dd, 1H), 2.53-2.42 (m, 3H), 2.29 (s, 3H), 2.21 (s, 3H), 1.99-1.90 (m, 2H), 1.85-1.75 (m, 2H), 1.73-1.62 (m, 2H); LC-MS (ESI) m/z 397.2 [M+H]⁺.

### Example 77: 1-(4-Chlorophenyl)-N-{[(3R)-2,6-dimethyl-1,2,3,4-tetrahydroisoquinolin-3-yl]methyl}cyclohexanecarboxamide (77)

The compound was prepared by general procedure i1 with Lix (40 mg, 0.21 mmol), 1-(4-chlorophenyl)-1-cyclohexanecarboxylic acid (50.2 mg, 0.21 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=98/2) to give 77 as a yellow liquid (61.6 mg, 71%). ¹H NMR (CDCl₃, 400 MHz) δ 7.34 (d, 2H), 7.29 (d, 2H), 6.94 (d, 1H), 6.84 (d, 1H), 6.83 (s, 1H), 6.02 (br s, 1H), 3.70 (d, 1H), 3.55 (d, 1H), 3.32 (dt, 1H), 3.23 (dt, 1H), 2.76-2.67 (m, 1H), 2.60 (dd, 1H), 2.48 (dd, 1H), 2.32-2.18 (m, 2H, overlapped with two s at 2.29 and 2.21), 2.29 (s, 3H), 2.21 (s, 3H), 1.96-1.86 (m, 2H), 1.65-1.48 (m, 5H), 1.43-1.31 (m, 1H); LC-MS (ESI) m/z 411.2 [M+H]⁺.

### Example 78: (2S)—N-{[(3R)-2,6-Dimethyl-1,2,3,4-tetrahydroisoquinolin-3-yl]methyl}-2,3-dihydro-1H-indole-2-carboxamide (78)

The compound was prepared by general procedure i1 with Lix (53.5 mg, 0.281 mmol), (S)-(−)-indoline-2-carboxylic acid (43.6 mg, 0.267 mmol), 1-(3-dimethylaminopropyl)-3-ethylcarbodiimide hydrochloride (80.8 mg, 0.422 mmol), 1-hydroxybenzotriazole hydrate (57 mg, 0.422 mmol) and N,N-diisopropylethylamine (0.122 mL, 0.703 mmol) in dichloromethane (2.8 mL), stirred at room temperature for 16 h. After workup, the crude was purified by flash column chromatography (SiO₂, dichloromethane/methanol=96/4) to give 78 as a yellow liquid (47.9 mg, 51%). ¹H NMR (CDCl₃, 400 MHz) δ 7.43 (br s, 1H), 7.12-7.03 (m, 2H), 6.98-6.88 (m, 3H), 6.80 (td, 1H), 6.71 (d, 1H), 4.40 (t, 1H), 4.25 (br s, 1H), 3.81 (d, 1H), 3.68 (d, 1H), 3.62-3.50 (m, 2H), 3.38-3.29 (m, 1H), 3.05 (dd, 1H), 2.94-2.86 (m, 1H), 2.78 (dd, 1H), 2.68 (dd, 1H), 2.41 (s, 3H), 2.29 (s, 3H); LC-MS (ESI) m/z 336.2 [M+H]⁺.

### Example 79: N-{[(3R)-2,6-Dimethyl-1,2,3,4-tetrahydroisoquinolin-3-yl]methyl}-9H-xanthene-9-carboxamide (79)

The compound was prepared by general procedure i1 with Lix (40 mg, 0.21 mmol), xanthene-9-carboxylic acid (47.6 mg, 0.21 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=98/2) to give 79 as a white solid (52 mg, 62%). ¹H NMR (CDCl₃, 400 MHz) δ 7.38 (ddd, 2H), 7.33-7.27 (m, 2H), 7.13 (d, 2H), 7.11-7.06 (m, 2H), 6.93 (d, 1H), 6.87 (d, 1H), 6.80 (s, 1H), 6.00 (br s, 1H), 4.87 (s, 1H), 3.64 (d, 1H), 3.52 (d, 1H), 3.32-3.16 (m, 2H), 2.75-2.65 (m, 1H), 2.59 (dd, 1H), 2.42 (dd, 1H), 2.28 (s, 3H), 2.18 (s, 3H); LC-MS (ESI) m/z 399.2 [M+H]⁺.

### Example 80: trans-N-{[(3R)-2,6-Dimethyl-1,2,3,4-tetrahydroisoquinolin-3-yl]methyl}-2-phenylcyclopropanecarboxamide (80)

The compound was prepared by general procedure i1 with Lix (40 mg, 0.21 mmol), trans-2-phenylcyclopropane-1-carboxylic acid (34.1 mg, 0.21 mmol), 1-(3-dimethylaminopropyl)-3-ethylcarbodiimide hydrochloride (60.4 mg, 0.315 mmol), 1-hydroxybenzotriazole hydrate (42.6 mg, 0.315 mmol) and N,N-diisopropylethylamine (91.5 μL, 0.526 mmol) in dichloromethane (2.1 mL), stirred at room temperature for 16 h. After workup, the crude was purified by flash column chromatography (SiO₂, dichloromethane/methanol=98/2) to give 80 as a yellow solid (42.6 mg, 61%). ¹H NMR (CDCl₃, 400 MHz) δ 7.31-7.29 (m, overlapped with CDCl₃, 2H), 7.24-7.12 (m, 1H), 7.09 (d, 2H), 6.99-6.89 (m, 3H), 6.39 (br, 1H), 3.82 (d, 1H), 3.69 (dd, 1H), 3.52 (ttt, 1H), 3.45-3.30 (m, 1H), 2.92-2.83 (m, 1H), 2.78-2.70 (m, 2H), 2.54-2.47 (m, 1H), 2.38 (d, 3H), 2.30 (d, 3H) 1.72-1.60 (m, 2H), 1.28-1.20 (m, 1H); LC-MS (ESI) m/z 335.2 [M+H]⁺.

### Example 81: 3-(3-Fluorophenyl)-N-(((3R)-6-methoxy-2-methyl-1,2,3,4-tetrahydroisoquinolin-3-yl)methyl)propanamide (81)

The compound was prepared by general procedure i1 with Mix (46 mg, 0.223 mmol), 3-(3-fluorophenyl) propionic acid (35.6 mg, 0.212 mmol), 1-(3-dimethylaminopropyl)-3-ethylcarbodiimide hydrochloride (64 mg, 0.335 mmol), 1-hydroxybenzotriazole hydrate (45.3 mg, 0.335 mmol) and N,N-diisopropylethylamine (136 μL, 0.781 mmol) in dichloromethane (4.5 mL), stirred at room temperature for 18 h. After workup, the crude was purified by flash column chromatography (5% methanol in dichloromethane) to give 81 as a white solid (47 mg, 59%). ¹H NMR (300 MHz, d₆-DMSO) δ 7.81 (s, 1H), 7.30 (q, 1H), 7.09-6.97 (m, 3H), 6.95 (d, 1H), 6.69 (dd, 1H), 6.59 (s, 1H), 3.74-3.61 (m, 4H), 3.46 (d, 1H), 3.31-3.25 (m overlapped with H₂O, 1H), 3.09-2.97 (m, 1H), 2.84 (t, 2H), 2.69-2.52 (m, 3H), 2.42 (t, 2H), 2.30 (s, 3H); LC-MS (ESI) m/z 357.2 [M+H]⁺.

### Example 82: N-(((3R)-6-Methoxy-2-methyl-1,2,3,4-tetrahydroisoquinolin-3-yl)methyl)-3-phenylpropanamide (82)

The compound was prepared by general procedure i1 with Mix (46 mg, 0.223 mmol), 3-phenylpropionic acid (32 mg, 0.212 mmol), 1-(3-dimethylaminopropyl)-3-ethylcarbodiimide hydrochloride (64 mg, 0.335 mmol), 1-hydroxybenzotriazole hydrate (45.3 mg, 0.335 mmol) and N,N-diisopropylethylamine (136 μL, 0.781 mmol) in dichloromethane (4.5 mL), stirred at room temperature for 18 h. After workup, the crude was purified by flash column chromatography (5% methanol in dichloromethane) to give 82 as a white solid (51 mg, 68%). ¹H NMR (300 MHz, d₆-DMSO) δ 7.79 (t, 1H), 7.31-7.11 (m, 5H), 6.95 (d, 1H), 6.69 (dd, 1H), 6.60 (s, 1H), 3.74-3.61 (m, 4H), 3.46 (d, 1H), 3.30-3.22 (m overlapped with H₂O, 1H), 3.09-2.96 (m, 1H), 2.81 (t, 2H), 2.70-2.52 (m, 3H), 2.41 (t, 2H), 2.31 (s, 3H); LC-MS (ESI) m/z 339.2 [M+H]⁺.

### Example 83: 3-Cyclohexyl-N-(((3R)-6-methoxy-2-methyl-1,2,3,4-tetrahydroisoquinolin-3-yl)methyl)propanamide (83)

The compound was prepared by general procedure i1 with Mix (46 mg, 0.223 mmol), 3-cyclohexylpropionic acid (33 μL, 0.212 mmol), 1-(3-dimethylaminopropyl)-3-ethylcarbodiimide hydrochloride (64 mg, 0.335 mmol), 1-hydroxybenzotriazole hydrate (45.3 mg, 0.335 mmol) and N,N-diisopropylethylamine (136 μL, 0.781 mmol) in dichloromethane (4.5 mL), stirred at room temperature for 18 h. After workup, the crude was purified by flash column chromatography (5% methanol in dichloromethane) to give 83 as a white solid (42 mg, 55%). ¹H NMR (300 MHz, d₆-DMSO) δ 7.74 (t, 1H), 6.95 (d, 1H), 6.69 (dd, 1H), 6.62 (s, 1H), 3.73-3.61 (m, 4H), 3.45 (d, 1H), 3.31-3.24 (m overlapped with H₂O, 1H), 3.02 (dt, 1H), 2.78-2.53 (m, 3H), 2.32 (s, 3H), 2.09 (t, 2H), 1.74-1.53 (m, 5H), 1.38 (q, 2H), 1.26-1.01 (m, 4H), 0.93-0.79 (m, 2H); LC-MS (ESI) m/z 345.3 [M+H]⁺.

### Example 84: N-(((3R)-6-Ethyl-2-methyl-1,2,3,4-tetrahydroisoquinolin-3-yl)methyl)-3-phenylpropanamide (84)

The compound was prepared by general procedure i1 Nix (50 mg, 0.245 mmol), 3-phenylpropionic acid (35 mg, 0.232 mmol), 1-(3-dimethylaminopropyl)-3-ethylcarbodiimide hydrochloride (70 mg, 0.368 mmol), 1-hydroxybenzotriazole hydrate (50 mg, 0.368 mmol) and N,N-diisopropylethylamine (150 μL, 0.858 mmol) in dichloromethane (5 mL), stirred at room temperature for 18 h. After workup, the crude was purified by flash column chromatography (3-5% methanol in dichloromethane) to give 84 as a beige solid (44 mg, 54%). ¹H NMR (300 MHz, d₆-DMSO) δ 7.80 (t, 1H), 7.30-7.12 (m, 5H), 6.98-6.84 (m, 3H), 3.70 (d, 1H), 3.48 (d, 1H), 3.29-3.22 (m overlapped with H₂O, 1H), 3.09-2.93 (m, 1H), 2.81 (t, 2H), 2.71-2.52 (m overlapped with do-DMSO, 5H), 2.40 (t, 2H), 2.31 (s, 3H), 1.15 (t, 3H); LC-MS (ESI) m/z 337.3 [M+H]⁺.

### Example 85: 3-Cyclohexyl-N-(((3R)-6-ethyl-2-methyl-1,2,3,4-tetrahydroisoquinolin-3-yl)methyl)propanamide (85)

The compound was prepared by general procedure i1 with Nix (50 mg, 0.245 mmol), 3-cyclohexylpropionic acid (36 μL, 0.232 mmol), 1-(3-dimethylaminopropyl)-3-ethylcarbodiimide hydrochloride (70 mg, 0.368 mmol), 1-hydroxybenzotriazole hydrate (50 mg, 0.368 mmol) and N,N-diisopropylethylamine (150 μL, 0.858 mmol) in dichloromethane (5 mL), stirred at room temperature for 18 h. After workup, the crude was purified by flash column chromatography (3-5% methanol in dichloromethane) to give 85 as a beige solid (54 mg, 64%). ¹H NMR (300 MHz, d₆-DMSO) δ 7.76 (t, 1H), 6.99-6.83 (m, 3H), 3.72 (d, 1H), 3.51 (d, 1H), 3.29-3.16 (m overlapped with H₂O, 1H), 3.09-2.93 (m, 1H), 2.78-2.44 (m overlapped with do-DMSO, 5H), 2.34 (s, 3H), 2.08 (t, 2H), 1.72-1.52 (m, 5H), 1.37 (q, 2H), 1.26-1.04 (m, 7H), 0.92-0.78 (m, 2H); LC-MS (ESI) m/z 343.3 [M+H]⁺.

### Example 86: 4-Ethyl-N-[(6-fluoro-2-methyl-1,2,3,4-tetrahydroisoquinolin-3-yl)methyl]benzamide (86)

The compound was prepared by general procedure i1 with Oix (60 mg, 0.309 mmol), 4-ethylbenzoic acid (51 mg, 0.34 mmol), 1-(3-dimethylaminopropyl)-3-ethylcarbodiimide hydrochloride (77 mg, 0.402 mmol), 1-hydroxybenzotriazole hydrate (45.9 mg, 0.34 mmol) and N,N-diisopropylethylamine (80.7 μL, 0.463 mmol) in dichloromethane (4.6 mL), stirred at room temperature for 16 h. After workup, the crude was purified by flash column chromatography (SiO₂, dichloromethane/methanol=98/2) to give 86 as a yellow liquid (54 mg, 54%). ¹H NMR (CDCl₃, 300 MHz) δ 7.69 (d, 2H), 7.23 (d, 2H), 6.99 (dd, 1H), 6.87-6.76 (m, 3H), 3.85 (d, 1H), 3.70-3.60 (m, 2H), 3.56-3.53 (m, 1H), 2.96-2.94 (m, 1H), 2.82-2.79 (m, 2H), 2.67 (q, 2H), 2.43 (s, 3H), 1.23 (t, 3H); LC-MS (ESI) m/z 327.2 [M+H]⁺.

### Example 87: 3,4-Dichloro-N-[(6-fluoro-2-methyl-1,2,3,4-tetrahydroisoquinolin-3-yl)methyl]benzamide (87)

The compound was prepared by general procedure i1 with Oix (60 mg, 0.309 mmol), 3,4-dichlorobenzoic acid (64.9 mg, 0.34 mmol), 1-(3-dimethylaminopropyl)-3-ethylcarbodiimide hydrochloride (77 mg, 0.402 mmol), 1-hydroxybenzotriazole hydrate (45.9 mg, 0.34 mmol) and N,N-diisopropylethylamine (80.7 μL, 0.463 mmol) in dichloromethane (4.6 mL), stirred at room temperature for 16 h. After workup, the crude was purified by flash column chromatography (SiO₂, dichloromethane/methanol=98/2) to give 87 as a yellow liquid (59.3 mg, 52%). ¹H NMR (CDCl₃, 300 MHz) δ 7.83 (s, 1H), 7.58 (d, 1H), 7.48 (d, 1H), 7.02 (dd, 1H), 6.90-6.79 (m, 3H), 3.88 (d, 1H), 3.75-3.63 (m, 2H), 3.49 (ddd, 1H), 3.03-3.00 (m, 1H), 2.85-2.78 (m, 2H), 2.44 (s, 3H); LC-MS (ESI) m/z 367.1 [M+H]⁺.

### Example 88: N-[(6-Fluoro-2-methyl-1,2,3,4-tetrahydroisoquinolin-3-yl)methyl]-4-(trifluoromethyl)benzamide (88)

The compound was prepared by general procedure i1 with Oix (60 mg, 0.309 mmol), 4-(trifluoromethyl)benzoic acid (64.6 mg, 0.34 mmol), 1-(3-dimethylaminopropyl)-3-ethylcarbodiimide hydrochloride (77 mg, 0.402 mmol), 1-hydroxybenzotriazole hydrate (45.9 mg, 0.34 mmol) and N,N-diisopropylethylamine (80.7 μL, 0.463 mmol) in dichloromethane (4.6 mL), stirred at room temperature for 16 h. After workup, the crude was purified by flash column chromatography (SiO₂, dichloromethane/methanol=98/2) to give 88 as a yellow liquid (51.1 mg, 45%). ¹H NMR (CDCl₃, 300 MHz) δ 7.89 (d, 2H), 7.68 (d, 2H), 7.05-7.00 (m, 2H), 6.90-6.80 (m, 2H), 3.90 (d, 1H), 3.77-3.68 (m, 2H), 3.53 (ddd, 1H), 3.09-3.04 (m, 1H), 2.88-2.78 (m, 2H), 2.46 (s, 3H); LC-MS (ESI) m/z 367.1 [M+H]⁺.

### Example 89: N-[(6-Fluoro-2-methyl-1,2,3,4-tetrahydroisoquinolin-3-yl)methyl]-3-(3-fluorophenyl)propanamide (89)

The compound was prepared by general procedure i1 with Oix (26.9 mg, 0.139 mmol), 3-(3-fluorophenyl) propionic acid (21 mg, 0.125 mmol), 1-(3-dimethylaminopropyl)-3-ethylcarbodiimide hydrochloride (53.1 mg, 0.277 mmol), benzotriazol-1-ol (37.4 mg, 0.277 mmol) and N,N-diisopropylethylamine (84.4 μL, 0.485 mmol) in dichloromethane (2 mL), stirred at room temperature for 16 h. After workup, the crude was purified by flash column chromatography (SiO₂, dichloromethane/methanol=98/2) to give 89 as a yellow liquid (18.9 mg, 44%). ¹H NMR (CDCl₃, 300 MHz) δ 7.22 (dd, 1H), 7.01-6.76 (m, 6H), 6.13 (br s, 1H), 3.79 (d, 1H), 3.64 (d, 1H), 3.47-3.41 (m, 1H), 3.37-3.30 (m, 1H), 2.97 (t, 2H), 2.85-2.43 (m, overlapped with t at 2.50, 5H), 2.33 (s, 3H); LC-MS (ESI) m/z 345.1 [M+H]⁺.

### Example 90: N-{[(3R)-6-Fluoro-2-methyl-1,2,3,4-tetrahydroisoquinolin-3-yl]methyl}-2,3-dihydro-1H-indene-2-carboxamide (90)

The compound was prepared by general procedure i1 with Pix (40 mg, 0.206 mmol), 2-indanecarboxylic acid (32.7 mg, 0.202 mmol), 1-(3-dimethylaminopropyl)-3-ethylcarbodiimide hydrochloride (59.2 mg, 0.309 mmol), 1-hydroxybenzotriazole hydrate (41.7 mg, 0.309 mmol) and N,N-diisopropylethylamine (89.7 μL, 0.515 mmol) in dichloromethane (2.1 mL), stirred at room temperature for 16 h. After workup, the crude was purified by flash column chromatography (SiO₂, dichloromethane/methanol=98/2) to give 90 as a yellow solid (51.1 mg, 75%). ¹H NMR (CDCl₃, 400 MHz) δ 7.22-7.12 (m, 4H), 6.94 (dd, 1H), 6.85 (td, 1H), 6.79 (dd, 1H), 6.32 (br s, 1H), 3.81 (d, 1H), 3.64 (d, 1H), 3.48 (dt, 1H), 3.40 (dt, 1H), 3.28-3.11 (m, 5H), 2.89-2.67 (m, 3H), 2.38 (s, 3H); LC-MS (ESI) m/z 339.1 [M+H]⁺.

### Example 91: N-{[(3R)-6-Fluoro-2-methyl-1,2,3,4-tetrahydroisoquinolin-3-yl]methyl}-1,2,3,4-tetrahydronaphthalene-2-carboxamide (91)

The compound was prepared by general procedure i1 with Pix (40 mg, 0.206 mmol), 1,2,3,4-tetrahydro-2-naphthoic acid (35.6 mg, 0.202 mmol), 1-(3-dimethylaminopropyl)-3-ethylcarbodiimide hydrochloride (59.2 mg, 0.309 mmol), 1-hydroxybenzotriazole hydrate (41.7 mg, 0.309 mmol) and N,N-diisopropylethylamine (89.7 μL, 0.515 mmol) in dichloromethane (2.80 mL), stirred at room temperature for 16 h. After workup, the crude was purified by flash column chromatography (SiO₂, dichloromethane/methanol=98/2) to give 91 as a yellow liquid (67 mg, 67%). ¹H NMR (CDCl₃, 400 MHz) δ 7.14-7.04 (m, 4H), 6.98 (t, 1H), 6.84 (td, 1H), 6.79 (dt, 1H), 6.29 (br s, 1H), 3.81 (dd, 1H), 3.64 (d, 1H), 3.52-3.35 (m, 2H), 3.03 (dd, 1H), 2.96-2.66 (m, 6H), 2.54 (dddd, 1H), 2.38 (s, 1.5H), 2.38 (s, 1.5H), 2.13-2.03 (m, 1H), 1.96-1.84 (m, 1H); LC-MS (ESI) m/z 353.1 [M+H]⁺.

### Example 92: trans-N-{[(3R)-6-Fluoro-2-methyl-1,2,3,4-tetrahydroisoquinolin-3-yl]methyl}-2-phenylcyclopropanecarboxamide (92)

The compound was prepared by general procedure i1 with Pix (40 mg, 0.206 mmol), trans-2-phenylcyclopropane-1-carboxylic acid (32.7 mg, 0.202 mmol), 1-(3-dimethylaminopropyl)-3-ethylcarbodiimide hydrochloride (59.2 mg, 0.309 mmol), 1-hydroxybenzotriazole hydrate (41.7 mg, 0.309 mmol) and N,N-diisopropylethylamine (89.7 μL, 0.515 mmol) in dichloromethane (2.1 mL), stirred at room temperature for 16 h. After workup, the crude was purified by flash column chromatography (SiO₂, dichloromethane/methanol=98/2) to give 92 as a yellow solid (55.6 mg, 81%). ¹H NMR (CDCl₃, 400 MHz) δ 7.30-7.24 (m, 2H), 7.21-7.15 (m, 1H), 7.10-7.06 (m, 2H), 6.98 (ddd, 1H), 6.87-6.76 (m, 2H), 6.41-6.33 (m, 1H), 3.81 (d, 1H), 3.66 (dd, 1H), 3.50 (tt, 1H), 3.45-3.32 (m, 1H), 2.91-2.82 (m, 1H), 2.81-2.68 (m, 2H), 2.53-2.46 (m, 1H), 2.38 (s, 1.5H), 2.38 (s, 1.5H), 1.76-1.59 (m, 2H), 1.28-1.21 (m, 1H); LC-MS (ESI) m/z 339.1 [M+H]⁺.

### Example 93: N-((6-Chloro-2-methyl-1,2,3,4-tetrahydroisoquinolin-3-yl)methyl)-4-ethylbenzamide (93)

The compound was prepared by general procedure i1 with Qix (47 mg, 0.223 mmol), 4-ethylbenzoic acid (33.5 mg, 0.223 mmol), 1-(3-dimethylaminopropyl)-3-ethylcarbodiimide hydrochloride (85.5 mg, 0.446 mmol), 1-hydroxybenzotriazole hydrate (60 mg, 0.446 mmol) and N,N-diisopropylethylamine (136 μL, 0.780 mmol) in dichloromethane (4.5 mL), stirred at room temperature for 18 h. After workup, the crude was purified by flash column chromatography (2% methanol in dichloromethane) to give 93 as a yellow syrup (37 mg, 49%). ¹H NMR (300 MHz, d₆-DMSO) δ 8.33 (t, 1H), 7.74 (d, 2H), 7.27 (d, 2H), 7.19-7.12 (m, 2H), 7.11-7.04 (m, 1H), 3.74 (d, 1H), 3.61-3.45 (m, 2H), 3.20-3.08 (m, 1H), 2.91 (quin, 1H), 2.81 (dd, 1H), 2.71-2.57 (m, 3H), 2.39 (s, 3H), 1.17 (t, 3H); LC-MS (ESI) m/z 343.1 [M+H]⁺.

### Example 94: N-((6-Chloro-2-methyl-1,2,3,4-tetrahydroisoquinolin-3-yl)methyl)-4-(ethylsulfanyl)benzamide (94)

The compound was prepared by general procedure i1 with Qix (47 mg, 0.223 mmol), 4-(ethylthio)benzoic acid (41 mg, 0.223 mmol), 1-(3-dimethylaminopropyl)-3-ethylcarbodiimide hydrochloride (85.5 mg, 0.446 mmol), 1-hydroxybenzotriazole hydrate (60 mg, 0.446 mmol) and N,N-diisopropylethylamine (136 μL, 0.78 mmol) in dichloromethane (4.5 mL), stirred at room temperature for 18 h. After workup, the crude was purified by flash column chromatography (2% methanol in dichloromethane) to give 94 as a white solid (45 mg, 54%). ¹H NMR (300 MHz, d₆-DMSO) δ 8.38 (t, 1H), 7.75 (d, 2H), 7.33 (d, 2H), 7.18-7.11 (m, 2H), 7.11-7.03 (m, 1H), 3.74 (d, 1H), 3.61-3.44 (m, 2H), 3.14 (dt, 1H), 3.03 (q, 2H), 2.91 (quin, 1H), 2.81 (dd, 1H), 2.65 (dd, 1H), 2.39 (s, 3H), 1.24 (t, 3H); LC-MS (ESI) m/z 375.0 [M+H]⁺.

### Example 95: 3,4-Dichloro-N-((6-chloro-2-methyl-1,2,3,4-tetrahydroisoquinolin-3-yl)methyl)benzamide (95)

The compound was prepared by general procedure i1 with Qix (47 mg, 0.223 mmol), 3,4-dichlorobenzoic acid (42.6 mg, 0.223 mmol), 1-(3-dimethylaminopropyl)-3-ethylcarbodiimide hydrochloride (85.5 mg, 0.446 mmol), 1-hydroxybenzotriazole hydrate (60 mg, 0.446 mmol) and N,N-diisopropylethylamine (136 μL, 0.78 mmol) in dichloromethane (4.5 mL), stirred at room temperature for 18 h. After workup, the crude was purified by flash column chromatography (2% methanol in dichloromethane) to give 95 as a white solid (49 mg, 58%). ¹H NMR (300 MHz, d₆-DMSO) δ 8.63 (t, 1H), 8.05 (d, 1H), 7.81 (dd, 1H), 7.76 (d, 1H), 7.22-7.13 (m, 2H), 7.13-7.05 (m, 1H), 3.75 (d, 1H), 3.63-3.48 (m, 2H), 3.23-3.11 (m, 1H), 2.94 (quin, 1H), 2.84 (dd, 1H), 2.67 (dd, 1H), 2.41 (s, 3H); LC-MS (ESI) m/z 384.9 [M+H]⁺.

### Example 96: N-((6-Chloro-2-methyl-1,2,3,4-tetrahydroisoquinolin-3-yl)methyl)-4-(trifluoromethyl)benzamide (96)

The compound was prepared by general procedure i1 with Qix (47 mg, 0.223 mmol), 4-(trifluoromethyl)benzoic acid (42 mg, 0.223 mmol), 1-(3-dimethylaminopropyl)-3-ethylcarbodiimide hydrochloride (85.5 mg, 0.446 mmol), 1-hydroxybenzotriazole hydrate (60 mg, 0.446 mmol) and N,N-diisopropylethylamine (136 μL, 0.78 mmol) in dichloromethane (4.5 mL), stirred at room temperature for 18 h. After workup, the crude was purified by flash column chromatography (2% methanol in dichloromethane) to give 96 as a white solid (40 mg, 47%). ¹H NMR (300 MHz, d₆-DMSO) δ 8.68 (t, 1H), 8.02 (d, 2H), 7.85 (d, 2H), 7.22-7.14 (m, 2H), 7.14-7.06 (m, 1H), 3.77 (d, 1H), 3.64-3.50 (m, 2H), 3.26-3.13 (m, 1H), 2.95 (quin, 1H), 2.85 (dd, 1H), 2.69 (dd, 1H), 2.42 (s, 3H); LC-MS (ESI) m/z 383.0 [M+H]⁺.

### Example 97: N-[(6-Chloro-2-methyl-1,2,3,4-tetrahydroisoquinolin-3-yl)methyl]-3-(3-fluorophenyl)propanamide (97)

The compound was prepared by general procedure i1 with Qix (72 mg, 0.342 mmol), 3-(3-fluorophenyl) propionic acid (16.3 mg, 0.092 mmol), 1-(3-dimethylaminopropyl)-3-ethylcarbodiimide hydrochloride (0.131 g, 0.683 mmol), 1-hydroxybenzotriazole hydrate (92.3 mg, 0.683 mmol) and N,N-diisopropylethylamine (0.208 mL, 1.2 mmol) in dichloromethane (5 mL), stirred at room temperature for 16 h. After workup, the crude was purified by flash column chromatography (SiO₂, dichloromethane/methanol=98/2) to give 97 as a brown solid (25.5 mg, 21%). ¹H NMR (CDCl₃, 300 MHz) δ 7.27-7.15 (m, overlapped with CDCl₃, 1H), 7.11 (d, 1H), 7.05 (s, 1H), 7.00-6.82 (m, 4H), 6.04 (br s, 1H), 3.77 (d, 1H), 3.61 (d, 1H), 3.42 (dt, 1H), 3.31 (dt, 1H), 2.96 (t, 2H), 2.84-2.70 (m, 1H), 2.69-2.41 (m, 4H), 2.31 (s, 3H); LC-MS (ESI) m/z 361.1 [M+H]⁺.

### Example 98: N-(((3R)-6-Chloro-2-methyl-1,2,3,4-tetrahydroisoquinolin-3-yl)methyl)-4-(trifluoromethyl)benzamide (98)

The compound was prepared by general procedure i1 with Rix (75 mg, 0.356 mmol), 4-(trifluoromethyl)benzoic acid (64 mg, 0.338 mmol), 1-(3-dimethylaminopropyl)-3-ethylcarbodiimide hydrochloride (102 mg, 0.534 mmol), 1-hydroxybenzotriazole hydrate (72 mg, 0.534 mmol) and N,N-diisopropylethylamine (220 μL, 1.25 mmol) in dichloromethane (7 mL), stirred at room temperature for 18 h. After workup, the crude was purified by flash column chromatography (3-5% methanol in dichloromethane) to give 98 as a white solid (49 mg, 38%). ¹H NMR (300 MHz, d₆-DMSO) δ 8.69 (t, 1H), 8.03 (d, 2H), 7.85 (d, 2H), 7.22-7.14 (m, 2H), 7.14-7.07 (m, 1H), 3.76 (d, 1H), 3.64-3.50 (m, 2H), 3.26-3.13 (m, 1H), 2.95 (quin, 1H), 2.85 (dd, 1H), 2.69 (dd, 1H), 2.41 (s, 3H); LC-MS (ESI) m/z 383.1 [M+H]⁺.

### Example 99: N-(((3R)-6-Chloro-2-methyl-1,2,3,4-tetrahydroisoquinolin-3-yl)methyl)-3-(3-fluorophenyl)propanamide (99)

The compound was prepared by general procedure i1 with Rix (75 mg, 0.356 mmol), 3-(3-fluorophenyl) propionic acid (57 mg, 0.338 mmol), 1-(3-dimethylaminopropyl)-3-ethylcarbodiimide hydrochloride (102 mg, 0.534 mmol), 1-hydroxybenzotriazole hydrate (72 mg, 0.534 mmol) and N,N-diisopropylethylamine (220 μL, 1.25 mmol) in dichloromethane (7 mL), stirred at room temperature for 18 h. After workup, the crude was purified by flash column chromatography (3-5% methanol in dichloromethane) to give 99 as a white solid (51 mg, 42%). ¹H NMR (300 MHz, d₆-DMSO) δ 7.82 (t, 1H), 7.29 (q, 1H), 7.16 (dd, 1H), 7.12-6.92 (m, 5H), 3.71 (d, 1H), 3.48 (d, 1H), 3.30-3.22 (m overlapped with H₂O, 1H), 3.07-2.93 (m, 1H), 2.83 (t, 2H), 2.71-2.52 (m, 3H), 2.41 (t, 2H), 2.30 (s, 3H); LC-MS (ESI) m/z 361.2 [M+H]⁺.

### Example 100: N-(((3R)-6-Chloro-2-methyl-1,2,3,4-tetrahydroisoquinolin-3-yl)methyl)-3-cyclohexylpropanamide (100)

The compound was prepared by general procedure i1 with Rix (50 mg, 0.237 mmol), 3-cyclohexylpropionic acid (35 μL, 0.225 mmol), 1-(3-dimethylaminopropyl)-3-ethylcarbodiimide hydrochloride (68 mg, 0.356 mmol), 1-hydroxybenzotriazole hydrate (48 mg, 0.356 mmol) and N,N-diisopropylethylamine (140 μL, 0.83 mmol) in dichloromethane (5 mL), stirred at room temperature for 18 h. After workup, the crude was purified by flash column chromatography (3% methanol in dichloromethane) to give 100 as a white solid (49 mg, 38%). ¹H NMR (300 MHz, d₆-DMSO) δ 7.75 (t, 1H), 7.19-7.11 (m, 2H), 7.08 (d, 1H), 3.73 (d, 1H), 3.51 (d, 1H), 3.30-3.23 (m overlapped with H₂O, 1H), 3.01 (dt, 1H), 2.81-2.55 (m, 3H), 2.34 (s, 3H), 2.08 (t, 2H), 1.73-1.54 (m, 5H), 1.38 (q, 2H), 1.25-1.03 (m, 4H), 0.93-0.74 (m, 2H); LC-MS (ESI) m/z 349.2 [M+H]⁺.

### Example 101: N-(((3R)-6-Chloro-2-methyl-1,2,3,4-tetrahydroisoquinolin-3-yl)methyl)-3-phenylpropanamide (101)

The compound was prepared by general procedure i1 with Rix (50 mg, 0.237 mmol), 3-phenylpropionic acid (34 mg, 0.225 mmol), 1-(3-dimethylaminopropyl)-3-ethylcarbodiimide hydrochloride (68 mg, 0.356 mmol), 1-hydroxybenzotriazole hydrate (48 mg, 0.356 mmol) and N,N-diisopropylethylamine (140 μL, 0.83 mmol) in dichloromethane (5 mL), stirred at room temperature for 18 h. After workup, the crude was purified by flash column chromatography (3% methanol in dichloromethane) to give 101 as a white solid (55 mg, 71%). ¹H NMR (300 MHz, d₆-DMSO) δ 7.79 (t, 1H), 7.31-7.12 (m, 6H), 7.12-7.03 (m, 2H), 3.71 (d, 1H), 3.49 (d, 1H), 3.31-3.24 (m overlapped with H₂O, 1H), 3.07-2.94 (m, 1H), 2.81 (t, 2H), 2.73-2.57 (m, 2H), 2.55 (d, 1H), 2.40 (t, 2H), 2.30 (s, 3H); LC-MS (ESI) m/z 343.1 [M+H]⁺.

### Example 102: N-{[(3R)-6-Chloro-2-methyl-1,2,3,4-tetrahydroisoquinolin-3-yl]methyl}-1,2,3,4-tetrahydronaphthalene-2-carboxamide (102)

The compound was prepared by general procedure i1 with Rix (63.3 mg, 0.3 mmol), 1,2,3,4-tetrahydro-2-naphthoic acid (52.9 mg, 0.3 mmol), 1-(3-dimethylaminopropyl)-3-ethylcarbodiimide hydrochloride (86.4 mg, 0.451 mmol), 1-hydroxybenzotriazole hydrate (60.9 mg, 0.451 mmol) and N,N-diisopropylethylamine (0.131 mL, 0.751 mmol) in dichloromethane (3 mL), stirred at room temperature for 16 h. After workup, the crude was purified by flash column chromatography (SiO₂, dichloromethane/methanol=98/2) to give 102 as a yellow liquid (89.1 mg, 81%). ¹H NMR (CDCl₃, 400 MHz) δ 7.13-7.04 (m, 6H), 6.96 (d, 1H), 6.21 (br s, 1H), 3.79 (dd, 1H), 3.64 (d, 1H), 3.50-3.34 (m, 2H), 3.02 (dd, 1H), 2.96-2.63 (m, 6H), 2.58-2.48 (m, 1H), 2.38 (s, 3H), 2.12-2.04 (m, 1H), 1.96-1.84 (m, 1H); LC-MS (ESI) m/z 369.1 [M+H]⁺.

### Example 103: trans-N-{[(3R)-6-Chloro-2-methyl-1,2,3,4-tetrahydroisoquinolin-3-yl]methyl}-2-phenylcyclopropanecarboxamide (103)

The compound was prepared by general procedure i1 with Rix (63.3 mg, 0.3 mmol), trans-2-phenylcyclopropane-1-carboxylic acid (46.3 mg, 0.285 mmol), 1-(3-dimethylaminopropyl)-3-ethylcarbodiimide hydrochloride (86.4 mg, 0.451 mmol), 1-hydroxybenzotriazole hydrate (60.9 mg, 0.451 mmol) and N,N-diisopropylethylamine (0.131 mL, 0.751 mmol) in dichloromethane (3 mL), stirred at room temperature for 16 h. After workup, the crude was purified by flash column chromatography (SiO₂, dichloromethane/methanol=98/2) to give 103 as a white solid (89.4 mg, 88%). ¹H NMR (CDCl₃, 400 MHz) δ 7.29-7.23 (m, overlapped with CDCl₃, 2H), 7.21-7.15 (m, 1H), 7.12-7.05 (m, 4H), 6.95 (dd, 1H), 6.36-6.32 (m, 1H), 3.80 (d, 1H), 3.65 (dd, 1H), 3.54-3.30 (m, 2H), 2.90-2.81 (m, 1H), 2.79-2.64 (m, 2H), 2.53-2.45 (m, 1H), 2.38 (s, 3H), 1.70-1.58 (m, 2H), 1.28-1.20 (m, 1H); LC-MS (ESI) m/z 355.1 [M+H]⁺.

### Example 104: N-{[(3R)-6-Chloro-2-methyl-1,2,3,4-tetrahydroisoquinolin-3-yl]methyl}-2,3-dihydro-1H-indene-2-carboxamide (104)

The compound was prepared by general procedure i1 with Rix (50.8 mg, 0.241 mmol), 2-indanecarboxylic acid (39.1 mg, 0.241 mmol), 1-(3-dimethylaminopropyl)-3-ethylcarbodiimide hydrochloride (69.3 mg, 0.362 mmol), 1-hydroxybenzotriazole hydrate (48.9 mg, 0.362 mmol) and N,N-diisopropylethylamine (0.105 mL, 0.603 mmol) in dichloromethane (2.4 mL), stirred at room temperature for 16 h. After workup, the crude was purified by flash column chromatography (SiO₂, dichloromethane/methanol=97/3) to give 104 as a white solid (70.5 mg, 82%). ¹H NMR (CDCl₃, 400 MHz) δ 7.22-7.06 (m, 6H), 6.96 (d, 1H), 6.16 (br s, 1H), 3.80 (d, 1H), 3.64 (d, 1H), 3.47 (dt, 1H), 3.39 (ddd, 1H), 3.27-3.11 (m, 5H), 2.90-2.82 (m, 1H), 2.77 (dd, 1H), 2.69 (dd, 1H), 2.38 (s, 3H); LC-MS (ESI) m/z 355.1 [M+H]⁺.

### Example 105: N-{[(3R)-6-Bromo-2-methyl-1,2,3,4-tetrahydroisoquinolin-3-yl]methyl}-2,3-dihydro-1H-indene-2-carboxamide (105)

The compound was prepared by general procedure i1 with Six (40 mg, 0.157 mmol), 2-indanecarboxylic acid (25.4 mg, 0.157 mmol), 1-(3-dimethylaminopropyl)-3-ethylcarbodiimide hydrochloride (45.1 mg, 0.235 mmol), 1-hydroxybenzotriazole hydrate (31.8 mg, 0.235 mmol) and N,N-diisopropylethylamine (68.3 μL, 0.392 mmol) in dichloromethane (1.6 mL), stirred at room temperature for 16 h. After workup, the crude was purified by flash column chromatography (SiO₂, dichloromethane/methanol=98/2) to give 105 as a brown solid (38.8 mg, 62%). ¹H NMR (CDCl₃, 400 MHz) δ 7.28-7.23 (m, overlapped with CDCl₃, 2H), 7.22-7.12 (m, 4H), 6.91 (d, 1H), 6.21 (br s, 1H), 3.79 (d, 1H), 3.63 (d, 1H), 3.47 (dt, 1H), 3.39 (ddd, 1H), 3.28-3.11 (m, 5H), 2.90-2.83 (m, 1H), 2.77 (dd, 1H), 2.70 (dd, 1H), 2.38 (s, 3H); LC-MS (ESI) m/z 401.1 [M+H]⁺.

### Example 106: N-((2,7-Dimethyl-1,2,3,4-tetrahydroisoquinolin-3-yl)methyl)-4-(trifluoromethyl)benzamide (106)

The compound was prepared by general procedure i1 with Tix (60 mg, 0.315 mmol), 4-(trifluoromethyl)benzoic acid (59.9 mg, 0.315 mmol), EDCI (78.3 mg, 0.41 mmol) and DIPEA (80 μL, 0.473 mmol) in CH₂Cl₂ (5 mL), stirred at room temperature for 17 h. After workup, the crude was purified by Isco Combi-Flash Companion column chromatography (0-5% methanol in CH₂Cl₂) to give 106 (40.9 mg, 36%). ¹H NMR (300 MHz, CDCl₃) δ 7.85 (d, 2H), 7.68 (d, 2H), 7.05-6.78 (m, 4H), 3.85 (d, 1H), 3.78-3.60 (m, 2H), 3.58-3.37 (m, 1H), 3.07-2.98 (m, 1H), 2.90-2.62 (m, 2H), 2.42 (s, 3H), 2.30 (s, 3H); LC-MS (ESI) m/z 363.1 [M+H]⁺.

### Example 107: N-((2,7-Dimethyl-1,2,3,4-tetrahydroisoquinolin-3-yl)methyl)-4-ethylbenzamide (107)

The compound was prepared by general procedure i1 with Tix (50 mg, 0.263 mmol), 4-ethylbenzoic acid (39.5 mg, 0.263 mmol), EDCI (65.3 mg, 0.346 mmol) and DIPEA (70 μL, 0.394 mmol) in CH₂Cl₂ (5 mL), stirred at room temperature for 17 h. After workup, the crude was purified by Isco Combi-Flash Companion column chromatography (0-5% methanol in CH₂Cl₂) to give 107 (37.7 mg, 44%). ¹H NMR (400 MHz, CDCl₃) δ 7.68 (d, 2H), 7.24 (d, 2H), 7.02-6.93 (m, 2H), 6.86 (s, 1H), 6.77 (br s, 1H), 3.84 (d, 1H), 3.77-3.62 (m, 2H), 3.50 (ddd, 1H), 3.02-2.90 (m, 1H), 2.83-2.75 (m, 2H), 2.68 (q, 2H), 2.42 (s, 3H), 2.30 (s, 3H), 1.24 (t, 3H); LC-MS (ESI) m/z 323.1 [M+H]⁺.

### Example 108: N-((2,7-Dimethyl-1,2,3,4-tetrahydroisoquinolin-3-yl)methyl)-4-(ethylsulfanyl)benzamide (108)

The compound was prepared by general procedure i1 with Tix (50 mg, 0.263 mmol), 4-(ethylsulfanyl)benzoic acid (47.9 mg, 0.263 mmol), EDCI (65.3 mg, 0.346 mmol) and DIPEA (70 μL, 0.394 mmol) in CH₂Cl₂ (5 mL), stirred at room temperature for 17 h. After workup, the crude was purified by Isco Combi-Flash Companion column chromatography (0-5% methanol in CH₂Cl₂) to give 108 (33.1 mg, 36%). ¹H NMR (400 MHz, CDCl₃) δ 7.66 (d, 2H), 7.29 (d, 2H), 7.01-6.95 (m, 2H), 6.86 (s, 1H), 6.76 (br s, 1H), 3.84 (d, 1H), 3.75-3.60 (m, 2H), 3.49 (dt, 1H), 3.09-2.89 (m, 3H), 2.86-2.68 (m, 2H), 2.42 (s, 3H), 2.30 (s, 3H), 1.34 (t, 3H); LC-MS (ESI) m/z 355.1 [M+H]⁺.

### Example 109: 3,4-Dichloro-N-((2,7-dimethyl-1,2,3,4-tetrahydroisoquinolin-3-yl)methyl)benzamide (109)

The compound was prepared by general procedure i2 with Tix (50 mg, 0.263 mmol), 3,4-dichlorobenzoyl chloride (55 mg, 0.263 mmol) and triethylamine (44.0 μL, 0.315 mmol) in CH₂Cl₂ (5 mL), stirred at room temperature for 2 h. After workup, the crude was purified by Isco Combi-Flash Companion column chromatography (0-5% methanol in CH₂Cl₂) to give 109 (67.7 mg, 71%). ¹H NMR (400 MHz, CDCl₃) δ 7.80 (d, 1H), 7.55 (dd, 1H), 7.48 (d, 1H), 7.02-6.97 (m, 2H), 6.88 (s, 1H), 6.78 (br s, 1H), 3.84 (d, 1H), 3.73 (d, 1H), 3.66 (dt, 1H), 3.47 (ddd, 1H), 3.06-2.96 (m, 1H), 2.83 (dd, 1H), 2.74 (dd, 1H), 2.42 (s, 3H), 2.30 (s, 3H); LC-MS (ESI) m/z 364.0 [M+H]⁺.

### Example 110: 4-(Ethylsulfanyl)-N-[(7-fluoro-2-methyl-1,2,3,4-tetrahydroisoquinolin-3-yl)methyl]benzamide (110)

The compound was prepared by general procedure i2 with Uix (50 mg, 0.257 mmol), 4-(ethylthio)benzoic acid (51.6 mg, 0.283 mmol), 1-(3-dimethylaminopropyl)-3-ethylcarbodiimide hydrochloride (64.1 mg, 0.335 mmol), benzotriazol-1-ol (38.3 mg, 0.283 mmol) and N,N-diisopropylethylamine (67.3 μL, 0.386 mmol) in dichloromethane (3.8 mL), stirred at room temperature for 16 h. After workup, the crude was purified by flash column chromatography (SiO₂, dichloromethane/methanol=98/2) to give 110 as a yellow liquid (41.9 mg, 45%). ¹H NMR (CDCl₃, 300 MHz) δ 7.68 (d, 2H), 7.29 (d, 2H), 7.03 (dd, 1H), 6.88-6.81 (m, 2H), 6.75 (dd, 1H), 3.85 (d, 1H), 3.75-3.61 (m, 2H), 3.49 (ddd, 1H), 3.02-2.94 (m, 3H), 2.79-2.74 (m, 2H), 2.42 (s, 3H), 1.34 (t, 3H); LC-MS (ESI) m/z 359.1 [M+H]⁺.

### Example 111: N-[(7-Fluoro-2-methyl-1,2,3,4-tetrahydroisoquinolin-3-yl)methyl]-4-(trifluoromethyl)benzamide (111)

The compound was prepared by general procedure i3 with Uix (70 mg, 0.36 mmol), 4-(trifluoromethyl)benzoic acid (75.4 mg, 0.396 mmol), triethylamine (75.3 μL, 0.541 mmol) and propylphosphonic anhydride solution (50 wt. % in ethyl acetate) (0.344 mL, 0.541 mmol) in dichloromethane (3.1 mL), stirred at room temperature for 16 h. After workup, the crude was purified by flash column chromatography (SiO₂, dichloromethane/methanol=98/2) to give 111 as a yellow liquid (64.1 mg, 49%). ¹H NMR (CDCl₃, 300 MHz) δ 7.87 (d, 2H), 7.67 (d, 2H), 7.05 (dd, 1H), 6.99 (br s, 1H)), 6.86 (td, 1H), 6.76 (dd, 1H), 3.86 (d, 1H), 3.79-3.64 (m, 2H), 3.49 (ddd, 1H), 3.09-2.99 (m, 1H), 2.95-2.65 (m, 2H), 2.42 (s, 3H); LC-MS (ESI) m/z 367.2 [M+H]⁺.

### Example 112: 4-Ethyl-N-[(7-fluoro-2-methyl-1,2,3,4-tetrahydroisoquinolin-3-yl)methyl]benzamide (112)

The compound was prepared by general procedure i1 with Uix (80 mg, 0.412 mmol), 4-ethylbenzoic acid (68 mg, 0.453 mmol), 1-(3-dimethylaminopropyl)-3-ethylcarbodiimide hydrochloride (0.103 g, 0.535 mmol), 1-hydroxybenzotriazole hydrate (61.2 mg, 0.453 mmol) and N,N-diisopropylethylamine (0.108 mL, 0.618 mmol) in dichloromethane (6 mL). After workup, the crude was purified by flash column chromatography (SiO₂, dichloromethane/methanol=98/2) to give 112 as a yellow liquid (40.1 mg, 30%). ¹H NMR (CDCl₃, 300 MHz) δ 7.70 (d, 2H), 7.24 (d, 2H), 7.04 (dd, 1H), 6.88-6.82 (m, 2H), 6.75 (dd, 1H), 3.86 (d, 1H), 3.75-3.63 (m, 2H), 3.55-3.46 (m, 1H), 3.03-2.98 (m, 1H), 2.80-2.72 (m, 2H), 2.68 (q, 2H), 2.42 (s, 3H), 1.24 (t, 3H); LC-MS (ESI) m/z 327.2 [M+H]⁺.

### Example 113: 3,4-Dichloro-N-[(7-fluoro-2-methyl-1,2,3,4-tetrahydroisoquinolin-3-yl)methyl]benzamide (113)

The compound was prepared by general procedure i1 with Uix (80 mg, 0.412 mmol), 3,4-dichlorobenzoic acid (86.5 mg, 0.453 mmol), 1-(3-dimethylaminopropyl)-3-ethylcarbodiimide hydrochloride (0.103 g, 0.535 mmol), 1-hydroxybenzotriazole hydrate (61.2 mg, 0.453 mmol) and N,N-diisopropylethylamine (0.108 mL, 0.618 mmol) in dichloromethane (6 mL). After workup, the crude was purified by flash column chromatography (SiO₂, dichloromethane/methanol=98/2) to give 113 as a yellow liquid (0.128 g, 84%). ¹H NMR (CDCl₃, 300 MHz) δ 7.84 (d, 1H), 7.58 (d, 1H), 7.48 (d, 1H), 7.05 (dd, 1H), 6.91-6.82 (m, 2H), 6.77 (dd, 1H), 3.87 (d, 1H), 3.75 (d, 1H), 3.67 (dt, 1H), 3.47 (ddd, 1H), 3.09-2.98 (m, 1H), 2.90-2.68 (m, 2H), 2.43 (s, 3H); LC-MS (ESI) m/z 367.0 [M+H]⁺.

### Example 114: N-((7-Chloro-2-methyl-1,2,3,4-tetrahydroisoquinolin-3-yl)methyl)-4-(trifluoromethyl)benzamide (114)

The compound was prepared by general procedure i1 with Vix (25 mg, 0.119 mmol), 4-(trifluoromethyl)benzoic acid (22.5 mg, 0.119 mmol), 1-(3-dimethylaminopropyl)-3-ethylcarbodiimide hydrochloride (45.6 mg, 0.238 mmol), 1-hydroxybenzotriazole hydrate (32.1 mg, 0.238 mmol) and N,N-diisopropylethylamine (50 μL, 0.298 mmol) in dichloromethane (2.4 mL), stirred at room temperature for 18 h. After workup, the crude was purified by flash column chromatography (3% methanol in dichloromethane) to give 114 as a yellow syrup (20 mg, 44%). ¹H NMR (300 MHz, d₆-DMSO) δ 8.68 (t, 1H), 8.03 (d, 2H), 7.85 (d, 2H), 7.21-7.15 (m, 2H), 7.12 (d, 1H), 3.79 (d, 1H), 3.67-3.49 (m, 2H), 3.28-3.14 (m overlapped with H₂O, 1H), 2.95 (quin, 1H), 2.82 (dd, 1H), 2.65 (dd, 1H), 2.40 (s, 3H); LC-MS (ESI) m/z 383.0 [M+H]⁺.

### Example 115: 3,4-Dichloro-N-((7-chloro-2-methyl-1,2,3,4-tetrahydroisoquinolin-3-yl)methyl)benzamide (115)

The compound was prepared by general procedure i1 with Vix (25 mg, 0.119 mmol), 3,4-dichlorobenzoic acid (22.7 mg, 0.119 mmol), 1-(3-dimethylaminopropyl)-3-ethylcarbodiimide hydrochloride (45.6 mg, 0.238 mmol), 1-hydroxybenzotriazole hydrate (32.1 mg, 0.238 mmol) and N,N-diisopropylethylamine (50 μL, 0.298 mmol) in dichloromethane (2.4 mL), stirred at room temperature for 18 h. After workup, the crude was purified by flash column chromatography (3% methanol in dichloromethane) to give 115 as a white foam (25 mg, 55%). ¹H NMR (300 MHz, d₆-DMSO) δ 8.64 (t, 1H), 8.05 (d, 1H), 7.82 (dd, 1H), 7.75 (d, 1H), 7.22-7.12 (m, 2H), 7.12 (d, 1H), 3.77 (d, 1H), 3.66-3.47 (m, 2H), 3.26-3.19 (m, 1H), 2.94 (quin, 1H), 2.81 (dd, 1H), 2.64 (dd, 1H), 2.39 (s, 3H); LC-MS (ESI) m/z 383.0 [M+H]⁺.

### N-((2-(Prop-2-en-1-yl)-1,2,3,4-tetrahydroisoquinolin-3-yl)methyl)-4-(trifluoromethyl)benzamide (116)

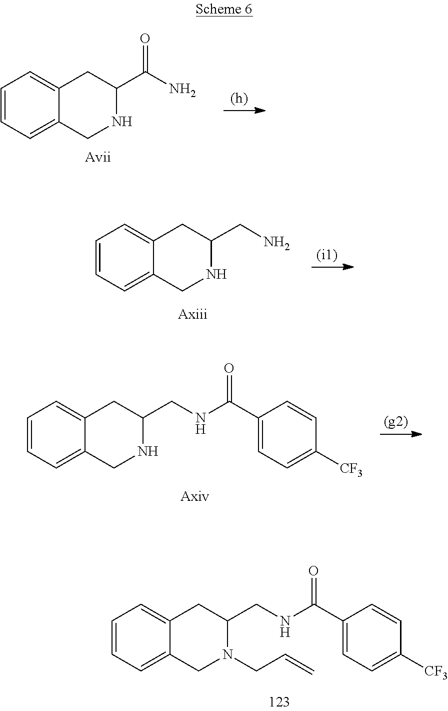 [SMILES: NC(=O)C1Cc2ccccc2CN1 ; NCC1Cc2ccccc2CN1 ; O=C(NCC1Cc2ccccc2CN1)c1ccc(C(F)(F)F)cc1 ; C=CCN1Cc2ccccc2CC1CNC(=O)c1ccc(C(F)(F)F)cc1]

### Step 1: 1,2,3,4-Tetrahydroisoquinolin-3-ylmethanamine (Axiii)

The compound was prepared by general procedure h with to a solution of 1,2,3,4-tetrahydroisoquinoline-3-carboxamide Avii (100 mg, 0.567 mmol) and lithium aluminium hydride (65 mg, 1.7 mmol) in tetrahydrofuran (16.2 mL), stirred at 50° C. for 4 h. After workup, the crude was purified by flash column chromatography (SiO₂, 20% methanol in dichloromethane then 20% methanol with 1% triethylamine in dichloromethane) to give Axiii as a beige syrup (60 mg, 49%). ¹H NMR (300 MHz, d₆-DMSO) δ 7.16-6.97 (m, 4H), 3.96-3.79 (m, 2H), 2.75-2.57 (m, 3H), 2.56-2.33 (m overlapped with d₆-DMSO, 2H), 2.12 (br s, 2H).

### Step 2: N-(1,2,3,4-Tetrahydroisoquinolin-3-ylmethyl)-4-(trifluoromethyl)benzamide (Axiv)

The compound was prepared by general procedure i1 with Axiii (60 mg, 0.37 mmol), 4-(trifluoromethyl)benzoic acid (70 mg, 0.37 mmol), 1-(3-dimethylaminopropyl)-3-ethylcarbodiimide hydrochloride (142 mg, 0.74 mmol), 1-hydroxybenzotriazole hydrate (100 mg, 0.74 mmol) and N,N-diisopropylethylamine (160 μL, 0.925 mmol) in dichloromethane (7.4 mL), stirred at room temperature for 18 h. After workup, the crude was purified by flash column chromatography (2% methanol in dichloromethane) to give Axiv as a brown syrup (40 mg, 32%). ¹H NMR (300 MHz, d₆-DMSO) δ 8.76 (t, 1H), 8.08 (d, 2H), 7.86 (d, 2H), 7.13-7.00 (m, 4H), 4.01-3.84 (m, 2H), 3.47-3.37 (m overlapped with H₂O, 2H), 3.12-3.00 (m, 1H), 2.76 (dd, 1H), 2.60-2.52 (m overlapped with do-DMSO, 1H).

### Step 3: Example 116: N-((2-(Prop-2-en-1-yl)-1,2,3,4-tetrahydroisoquinolin-3-yl)methyl)-4-(trifluoromethyl)benzamide (116)

The compound was prepared by general procedure g2 with Axiv (60 mg, 0.37 mmol), potassium carbonate (20 mg, 1.44 mmol) and 3-bromopropene (110 μL, 0.126 mmol) in acetonitrile (3 mL) and tetrahydrofuran (3 mL), stirred at 60° C. for 18 h. After workup, the crude was purified by flash column chromatography (2% methanol in dichloromethane) to give 116 as an orange syrup (26 mg, 58%). ¹H NMR (300 MHz, d₆-DMSO) δ 8.74-8.63 (m, 1H), 8.00 (d, 2H), 7.83 (d, 2H), 7.15-7.07 (m, 3H), 7.07-7.01 (m, 1H), 5.84 (ddt, 1H), 5.18 (dd, 1H), 5.10 (dd, 1H), 3.78 (d, 1H), 3.66 (d, 1H), 3.57-3.45 (m, 1H), 3.27-3.07 (m overlapped with H₂O, 4H), 2.88 (dd, 1H), 2.65 (dd, 1H); LC-MS (ESI) m/z 375.1 [M+H]⁺.

### Example 117: 4-(Ethylsulfanyl)-N-((2-(prop-2-en-1-yl)-1,2,3,4-tetrahydroisoquinolin-3-yl)methyl)benzamide (117)

The compound was prepared by general procedure i1 with Wix (65 mg, 0.321 mmol), 4-(ethylthio)benzoic acid (58.5 mg, 0.321 mmol), 1-(3-dimethylaminopropyl)-3-ethylcarbodiimide hydrochloride (123 mg, 0.642 mmol), 1-hydroxybenzotriazole hydrate (87 mg, 0.642 mmol) and N,N-diisopropylethylamine (140 μL, 0.803 mmol) in dichloromethane (6.4 mL), stirred at room temperature for 18 h. After workup, the crude was purified by flash column chromatography (1.5% methanol in dichloromethane) to give 117 as a yellow syrup (40 mg, 34%). ¹H NMR (300 MHz, d₆-DMSO) δ 8.41 (t, 1H), 7.77 (d, 2H), 7.35 (d, 2H), 7.16-7.09 (m, 3H), 7.08-7.02 (m, 1H), 5.86 (ddt, 1H), 5.19 (dd, 1H), 5.11 (dd, 1H), 3.79 (d, 1H), 3.66 (d, 1H), 3.55-3.43 (m, 1H), 3.30-3.09 (m, 4H), 3.04 (q, 2H), 2.87 (dd, 1H), 2.65 (dd, 1H), 1.26 (t, 3H); LC-MS (ESI) m/z 367.1 [M+H]⁺.

### Example 118: 3,4-Dichloro-N-((2-(prop-2-en-1-yl)-1,2,3,4-tetrahydroisoquinolin-3-yl)methyl)benzamide (118)

The compound was prepared by general procedure i1 with Wix (65 mg, 0.321 mmol), 3,4-dichlorobenzoic acid (61 mg, 0.321 mmol), 1-(3-dimethylaminopropyl)-3-ethylcarbodiimide hydrochloride (123 mg, 0.642 mmol), 1-hydroxybenzotriazole hydrate (87 mg, 0.642 mmol) and N,N-diisopropylethylamine (140 μL, 0.803 mmol) in dichloromethane (6.4 mL), stirred at room temperature for 18 h. After workup, the crude was purified by flash column chromatography (1.5% methanol in dichloromethane) to give 118 as a yellow syrup (46 mg, 38%). ¹H NMR (300 MHz, d₆-DMSO) δ 8.69-8.62 (m, 1H), 8.04 (d, 1H), 7.81 (dd, 1H), 7.75 (d, 1H), 7.17-7.09 (m, 3H), 7.09-7.02 (m, 1H), 5.86 (ddt, 1H), 5.20 (dd, 1H), 5.12 (dd, 1H), 3.78 (d, 1H), 3.67 (d, 1H), 3.58-3.43 (m, 1H), 3.29-3.09 (m overlapped with H₂O, 4H), 2.89 (dd, 1H), 2.66 (dd, 1H); LC-MS (ESI) m/z 377.0 [M+H]⁺.

### Example 119: N-((2-(3-Methylbut-2-en-1-yl)-1,2,3,4-tetrahydroisoquinolin-3-yl)methyl)-4-(trifluoromethyl)benzamide (119)

The compound was prepared by general procedure i1 with Xix (65 mg, 0.282 mmol), 4-(trifluoromethyl)benzoic acid (51 mg, 0.268 mmol), 1-(3-dimethylaminopropyl)-3-ethylcarbodiimide hydrochloride (81 mg, 0.423 mmol), 1-hydroxybenzotriazole hydrate (57 mg, 0.423 mmol) and N,N-diisopropylethylamine (172 μL, 0.988 mmol) in dichloromethane (5.6 mL), stirred at room temperature for 18 h. After workup, the crude was purified by flash column chromatography (1.5% methanol in dichloromethane) to give 119 as a beige solid (81 mg, 71%). ¹H NMR (300 MHz, d₆-DMSO) δ 8.73-8.63 (m, 1H), 8.00 (d, 2H), 7.83 (d, 2H), 7.15-7.07 (m, 3H), 7.07-7.00 (m, 1H), 5.28-5.18 (m, 1H), 3.75 (d, 1H), 3.64 (d, 1H), 3.54-3.48 (m, 1H), 3.27-3.10 (m overlapped with H₂O, 4H), 2.86 (d, 1H), 2.65 (d, 1H), 1.67 (s, 3H), 1.55 (s, 3H); LC-MS (ESI) m/z 403.1 [M+H]⁺.

### Example 120: 4-Ethyl-N-((2-(3-methylbut-2-en-1-yl)-1,2,3,4-tetrahydroisoquinolin-3-yl)methyl)benzamide (120)

The compound was prepared by general procedure i1 with Xix (65 mg, 0.282 mmol), 4-ethylbenzoic acid (40 mg, 0.268 mmol), 1-(3-dimethylaminopropyl)-3-ethylcarbodiimide hydrochloride (81 mg, 0.423 mmol), 1-hydroxybenzotriazole hydrate (57 mg, 0.423 mmol) and N,N-diisopropylethylamine (172 μL, 0.988 mmol) in dichloromethane (5.6 mL), stirred at room temperature for 18 h. After workup, the crude was purified by flash column chromatography (1.5% methanol in dichloromethane) to give 120 as a yellow syrup (51 mg, 50%). ¹H NMR (300 MHz, d₆-DMSO) δ 8.44-8.29 (m, 1H), 7.75 (d, 2H), 7.29 (d, 2H), 7.18-7.08 (m, 3H), 7.08-7.01 (m, 1H), 5.25 (br t, 1H), 3.77 (d, 1H), 3.65 (d, 1H), 3.53-3.48 (m, 1H), 3.29-3.05 (m overlapped with H₂O, 4H), 2.86 (d, 1H), 2.75-2.56 (m, 3H), 1.70 (s, 3H), 1.57 (s, 3H), 1.19 (t, 3H); LC-MS (ESI) m/z 363.2 [M+H]⁺.

### Example 121: 4-(Ethylsulfanyl)-N-((2-(3-methylbut-2-en-1-yl)-1,2,3,4-tetrahydroisoquinolin-3-yl)methyl)benzamide (121)

The compound was prepared by general procedure i1 with (Xix (65 mg, 0.282 mmol), 4-(ethylthio)benzoic acid (49 mg, 0.268 mmol), 1-(3-dimethylaminopropyl)-3-ethylcarbodiimide hydrochloride (81 mg, 0.423 mmol), 1-hydroxybenzotriazole hydrate (57 mg, 0.423 mmol) and N,N-diisopropylethylamine (172 μL, 0.988 mmol) in dichloromethane (5.6 mL), stirred at room temperature for 18 h. After workup, the crude was purified by flash column chromatography (1.5% methanol in dichloromethane) to give 121 as a yellow syrup (59 mg, 56%). ¹H NMR (300 MHz, d₆-DMSO) δ 8.47-8.36 (m, 1H), 7.77 (d, 2H), 7.35 (d, 2H), 7.16-7.08 (m, 3H), 7.08-7.02 (m, 1H), 5.29-5.19 (m, 1H), 3.77 (d, 1H), 3.65 (d, 1H), 3.52-3.48 (m, 1H), 3.28-3.10 (m overlapped with H₂O, 4H), 3.04 (q, 2H), 2.88 (dd, 1H), 2.67 (dd, 1H), 1.69 (s, 3H), 1.56 (s, 3H), 1.26 (t, 3H); LC-MS (ESI) m/z 395.1 [M+H]⁺.

### Example 122: 3,4-Dichloro-N-((2-(3-methylbut-2-en-1-yl)-1,2,3,4-tetrahydroisoquinolin-3-yl)methyl)benzamide (122)

The compound was prepared by general procedure i1 with Xix (65 mg, 0.282 mmol), 3,4-dichlorobenzoic acid (51 mg, 0.268 mmol), 1-(3-dimethylaminopropyl)-3-ethylcarbodiimide hydrochloride (81 mg, 0.423 mmol), 1-hydroxybenzotriazole hydrate (57 mg, 0.423 mmol) and N,N-diisopropylethylamine (172 μL, 0.988 mmol) in dichloromethane (5.6 mL), stirred at room temperature for 18 h. After workup, the crude was purified by flash column chromatography (1.5% methanol in dichloromethane) to give 122 as a yellow syrup (86 mg, 80%). ¹H NMR (300 MHz, d₆-DMSO) δ 8.72-8.60 (m, 1H), 8.04 (d, 1H), 7.81 (dd, 1H), 7.75 (d, 1H), 7.17-7.08 (m, 3H), 7.08-7.01 (m, 1H), 5.24 (br t, 1H), 3.76 (d, 1H), 3.65 (d, 1H), 3.53-3.47 (m, 1H), 3.28-3.07 (m overlapped with H₂O, 4H), 2.87 (d, 1H), 2.65 (d, 1H), 1.69 (s, 3H), 1.57 (s, 3H); LC-MS (ESI) m/z 403.0 [M+H]⁺.

### Example 123: N-{[2-(2-Methoxyethyl)-1,2,3,4-tetrahydroisoquinolin-3-yl]methyl}-4-(trifluoromethyl)benzamide (123)

The compound was prepared by general procedure i3 with Yix (86.4 mg, 0.392 mmol), 4-(trifluoromethyl)benzoic acid (82 mg, 0.431 mmol), triethylamine (82 μL, 0.588 mmol) and propylphosphonic anhydride solution (50 wt. % in ethyl acetate) (0.374 mL, 0.588 mmol) in dichloromethane (3.3 mL). After workup, the crude was purified by flash column chromatography (SiO₂, dichloromethane/methanol=98/2) to give 123 as a yellow liquid (98.1 mg, 64%). ¹H NMR (CDCl₃, 300 MHz) δ 7.95 (d, 2H), 7.80-7.67 (m, overlapped with d at 7.68, 3H), 7.18-7.04 (m, 4H), 4.00-3.84 (m, 3H), 3.58-3.54 (m, 1H), 3.49-3.46 (m, 1H), 3.35-3.26 (m, overlapped with s at 3.35, 5H), 2.85-2.77 (m, 2H), 2.71-2.62 (m, 2H); LC-MS (ESI) m/z 393.2 [M+H]⁺.

### Example 124: 4-(Ethylsulfanyl)-N-{[2-(2-methoxyethyl)-1,2,3,4-tetrahydroisoquinolin-3-yl]methyl}benzamide (124)

The compound was prepared by general procedure i3 with Yix (86.4 mg, 0.392 mmol), 4-(ethylthio)benzoic acid (78.6 mg, 0.431 mmol), triethylamine (82 μL, 0.588 mmol) and propylphosphonic anhydride solution (50 wt. % in ethyl acetate) (0.374 mL, 0.588 mmol) in dichloromethane (3.3 mL). After workup, the crude was purified by flash column chromatography (SiO₂, dichloromethane/methanol=98/2) to give 124 as a yellow liquid (67.8 mg, 45%). ¹H NMR (CDCl₃, 300 MHz) δ 7.75 (d, 2H), 7.55 (br s, 1H), 7.29 (d, 2H), 7.16-7.03 (m, 4H), 3.97 (d, 1H), 3.88-3.83 (m, 2H), 3.59-3.53 (m, 1H), 3.49-3.43 (m, 1H), 3.34-3.22 (m, overlapped with s at 3.34, 5H), 2.98 (q, 2H), 2.85-2.76 (m, 2H), 2.70-2.61 (m, 2H), 1.34 (t, 3H); LC-MS (ESI) m/z 385.2 [M+H]⁺.

### Example 125: 3,4-Dichloro-N-{[2-(2-methoxyethyl)-1,2,3,4-tetrahydroisoquinolin-3-yl]methyl}benzamide (125)

The compound was prepared by general procedure i3 with Yix (86.4 mg, 0.392 mmol), 3,4-dichlorobenzoic acid (82.4 mg, 0.431 mmol), triethylamine (82 μL, 0.588 mmol) and propylphosphonic anhydride solution (50 wt. % in ethyl acetate) (0.374 mL, 0.588 mmol) in dichloromethane (3.3 mL). After workup, the crude was purified by flash column chromatography (SiO₂, dichloromethane/methanol=98/2) to give 125 as a yellow liquid (86.7 mg, 56%). ¹H NMR (CDCl₃, 300 MHz) δ 7.92 (s, 1H), 7.72-7.69 (m, overlapped with d at 7.71, 2H), 7.50 (d, 1H), 7.18-7.04 (m, 4H), 4.00-3.84 (m, 3H), 3.61-3.55 (m, 1H), 3.50-3.43 (m, 1H), 3.37 (s, 3H), 3.27-3.24 (m, 2H), 2.84-2.76 (m, 2H), 2.69-2.62 (m, 2H); LC-MS (ESI) m/z 393.1 [M+H]⁺.

### Example 126: 3,4-Dichloro-N-((2-methyl-1,2,3,4-tetrahydroisoquinolin-3-yl)methyl)benzamide (126)

The compound was prepared by general procedure i1 with Aix (70 mg, 0.397 mmol), 3,4-dichlorobenzoic acid (72.1 mg, 0.377 mmol), EDCI (98.7 mg, 0.516 mmol), HOBt (59 mg, 0.437 mmol) and DIPEA (98 μL, 0.596 mmol) in CH₂Cl₂ (4 mL), stirred at room temperature for 17 h. After workup, the crude was purified by Isco Combi-Flash Companion column chromatography (0-5% methanol in CH₂Cl₂) to give 126 (100 mg, 72%). ¹H NMR (400 MHz, DMSO-d₆) δ 8.65 (br t, 1H), 8.07 (d, 1H), 7.83 (dd, 1H), 7.76 (d, 1H), 7.17-7.01 (m, 4H), 3.78 (d, 1H), 3.65-3.51 (m, 2H), 3.21 (ddd, 1H), 2.93 (ddd, 1H), 2.84 (dd, 1H), 2.68 (dd, 1H), 2.42 (s, 3H); LC-MS (ESI) m/z 350.1 [M+H]⁺.

### Example 127: N-((2-Methyl-1,2,3,4-tetrahydroisoquinolin-3-yl)methyl)-3-phenylpropanamide (127)

The compound was prepared by general procedure i1 with Aix (53 mg, 0.301 mmol), 3-phenylpropanoic acid (42.9 mg, 0.286 mmol), EDCI (74.7 mg, 0.391 mmol), HOBt (44.7 mg, 0.331 mmol) and DIPEA (75 μL, 0.451 mmol) in CH₂Cl₂ (4 mL), stirred at room temperature for 17 h. After workup, the crude was purified by Isco Combi-Flash Companion column chromatography (0-5% methanol in CH₂Cl₂) to give 127 (71.4 mg, 77%). ¹H NMR (400 MHz, DMSO-d₆) δ 7.79 (br t, 1H), 7.32-7.13 (m, 5H), 7.13-7.07 (m, 2H), 7.07-6.99 (m, 2H), 3.72 (d, 1H), 3.51 (d, 1H), 3.38-3.27 (m overlapped with H₂O, 1H), 3.08-2.97 (m, 1H), 2.81 (t, 2H), 2.72-2.53 (m, 3H), 2.40 (t, 2H), 2.31 (s, 3H); LC-MS (ESI) m/z 309.1 [M+H]⁺.

### Example 128: 3-(Cyclohexyl-N-((2-methyl-1,2,3,4-tetrahydroisoquinolin-3-yl)methyl)propenamide (128)

The compound was prepared by general procedure i1 with Aix (53 mg, 0.301 mmol), 3-cyclohexylpropanoic acid (30.5 μL, 0.296 mmol), EDCI (74.7 mg, 0.391 mmol), HOBt (44.7 mg, 0.331 mmol) and DIPEA (75 μL, 0.451 mmol) in CH₂Cl₂ (4 mL), stirred at room temperature for 17 h. After workup, the crude was purified by Isco Combi-Flash Companion column chromatography (0-5% methanol in CH₂Cl₂) to give 128 (69.8 mg, 74%). ¹H NMR (400 MHz, DMSO-d₆) δ 7.76 (br t, 1H), 7.16-7.01 (m, 4H), 3.75 (d, 1H), 3.53 (d, 1H), 3.39-3.26 (m overlapped with H₂O, 1H), 3.10-2.96 (m, 1H), 2.79-2.57 (m, 3H), 2.35 (s, 3H), 2.10 (dd, 2H), 1.72-1.55 (m, 5H), 1.39 (q, 2H), 1.26-1.08 (m, 4H), 0.91-0.77 (m, 2H); LC-MS (ESI) m/z 315.2 [M+H]⁺.

### Example 129: 3-(Cyclobutyl-N-((2-methyl-1,2,3,4-tetrahydroisoquinolin-3-yl)methyl)propanamide (129)

The compound was prepared by general procedure i1 with Aix (53 mg, 0.301 mmol), 3-cyclobutylpropanoic acid (33 μL, 0.296 mmol), EDCI (74.7 mg, 0.391 mmol), HOBt (44.7 mg, 0.331 mmol) and DIPEA (75 μL, 0.451 mmol) in CH₂Cl₂ (4 mL), stirred at room temperature for 17 h. After workup, the crude was purified by Isco Combi-Flash Companion column chromatography (0-5% methanol in CH₂Cl₂) to give 129 (72.6 mg, 84%). ¹H NMR (400 MHz, DMSO-d₆) δ 7.74 (br t, 1H), 7.16-7.00 (m, 4H), 3.74 (d, 1H), 3.53 (d, 1H), 3.39-3.24 (m overlapped with H₂O, 1H), 3.10-2.94 (m, 1H), 2.80-2.56 (m, 3H), 2.34 (s, 3H), 2.26-2.11 (m, 1H), 2.06-1.90 (m, 4H), 1.85-1.68 (m, 2H), 1.63-1.48 (m, 4H); LC-MS (ESI) m/z 287.2 [M+H]⁺.

### Example 130: (2R)—N-((2-Methyl-1,2,3,4-tetrahydroisoquinolin-3-yl)methyl)-1,2,3,4-tetrahydronaphthalene-2-carboxamide (130)

The compound was prepared by general procedure i1 with Aix (53 mg, 0.301 mmol), (2R)-1,2,3,4-tetrahydronaphthalene-2-carboxylic acid (50.3 mg, 0.286 mmol), EDCI (74.7 mg, 0.391 mmol), HOBt (44.7 mg, 0.331 mmol) and DIPEA (75 μL, 0.451 mmol) in CH₂Cl₂ (4 mL), stirred at room temperature for 17 h. After workup, the crude was purified by Isco Combi-Flash Companion column chromatography (0-5% methanol in CH₂Cl₂) to give 130 (61.4 mg, 61%). ¹H NMR (400 MHz, DMSO-d₆) δ 7.89 (br t, 1H), 7.18-7.01 (m, 8H), 3.77 (d, 1H), 3.55 (d, 1H), 3.48-3.34 (m, 1H), 3.09 (ddd, 1H), 2.93-2.62 (m, 7H), 2.62-2.53 (m, 1H), 2.37 (s, 3H), 2.02-1.88 (m, 1H), 1.80-1.61 (m, 1H); LC-MS (ESI) m/z 335.2 [M+H]⁺.

### Example 131: (2S)—N-((2-Methyl-1,2,3,4-tetrahydroisoquinolin-3-yl)methyl)-1,2,3,4-tetrahydronaphthalene-2-carboxamide (131)

The compound was prepared by general procedure i1 with Aix (70 mg, 0.397 mmol), (2S)-1,2,3,4-tetrahydronaphthalene-2-carboxylic acid (66.5 mg, 0.377 mmol), EDCI (98.7 mg, 0.516 mmol), HOBt (59 mg, 0.437 mmol) and DIPEA (98 μL, 0.596 mmol) in CH₂Cl₂ (4 mL), stirred at room temperature for 17 h. After workup, the crude was purified by Isco Combi-Flash Companion column chromatography (0-5% methanol in CH₂Cl₂) to give 131 (97.8 mg, 74%). ¹H NMR (400 MHz, DMSO-d₆) δ 7.88 (br t, 1H), 7.17-7.03 (m, 8H), 3.77 (d, 1H), 3.55 (d, 1H), 3.48-3.35 (m, 1H), 3.09 (ddd, 1H), 2.92-2.63 (m, 7H), 2.62-2.52 (m, 1H), 2.37 (s, 3H), 2.02-1.88 (m, 1H), 1.77-1.62 (m, 1H); LC-MS (ESI) m/z 335.2 [M+H]⁺.

### Example 132: N-((2-Methyl-1,2,3,4-tetrahydroisoquinolin-3-yl)methyl)-2,3-dihydro-1H-indene-2-carboxamide (132)

The compound was prepared by general procedure i1 with Aix (300 mg, 1.7 mmol), 2-indanecarboxylic acid (248 mg, 1.53 mmol), 1-(3-dimethylaminopropyl)-3-ethylcarbodiimide hydrochloride (489 mg, 2.55 mmol), 1-hydroxybenzotriazole hydrate (345 mg, 2.55 mmol) and N,N-diisopropylethylamine (1.04 mL, 5.96 mmol) in dichloromethane (17 mL). The crude was purified by flash column chromatography (SiO₂, 3% methanol in dichloromethane) to give 132 as a white solid (427 mg, 87%). ¹H NMR (600 MHz, Acetone-d₆) δ 7.21-7.14 (m, 3H), 7.13-7.09 (m, 4H), 7.09-7.06 (m, 1H), 7.06-7.02 (m, 1H), 3.82 (d, 1H), 3.59 (d, 1H), 3.52 (ddd, 1H), 3.34-3.27 (m, 2H), 3.20 (ddd, 2H), 3.10 (dd, 2H), 2.84-2.72 (m, 3H), 2.41 (s, 3H); LC-MS (ESI) m/z 321.2 [M+H]⁺.

### Example 133: N-((2-Methyl-1,2,3,4-tetrahydroisoquinolin-3-yl)methyl)-1H-indene-2-carboxamide (133)

The compound was prepared by general procedure i1 with Aix (76 mg, 0.431 mmol), 1H-indene-2-carboxylic acid (65.6 mg, 0.41 mmol), EDCI (107 mg, 0.516 mmol), HOBt (64.1 mg, 0.474 mmol) and DIPEA (110 μL, 0.647 mmol) in CH₂Cl₂ (4 mL), stirred at room temperature for 17 h. After workup, the crude was purified by Isco Combi-Flash Companion column chromatography (0-5% methanol in CH₂Cl₂) to give 133 (109 mg, 79%). ¹H NMR (400 MHz, DMSO-d₆) δ 8.13 (br t, 1H), 7.59-7.49 (m, 3H), 7.38-7.25 (m, 2H), 7.18-7.03 (m, 4H), 3.78 (d, 1H), 3.66 (d, 2H), 3.59 (d, 1H), 3.52 (dt, 1H), 3.22-3.11 (m, 1H), 2.95-2.79 (m, 2H), 2.69 (dd, 1H), 2.42 (s, 3H); LC-MS (ESI) m/z 319.1 [M+H]⁺.

### Example 134: 4-Ethyl-N-(((3R)-2-methyl-1,2,3,4-tetrahydroisoquinolin-3-yl)methyl)benzamide (134)

The compound was prepared by general procedure i1 with Bix (61.6 mg, 0.349 mmol), 4-ethylbenzoic acid (49.9 mg, 0.332 mmol), EDCI (86.8 mg, 0.454 mmol), HOBt (51.9 mg, 0.384 mmol) and DIPEA (87 μL, 0.524 mmol) in CH₂Cl₂ (4 mL), stirred at room temperature for 17 h. After workup, the crude was purified by Isco Combi-Flash Companion column chromatography (0-5% methanol in CH₂Cl₂) to give 134 (88.6 mg, 82%). ¹H NMR (400 MHz, DMSO-d₆) δ 8.35 (br t, 1H), 7.77 (d, 2H), 7.30 (d, 2H), 7.16-7.02 (m, 4H), 3.78 (d, 1H), 3.65-3.48 (m, 2H), 3.20 (ddd, 1H), 2.92 (ddd, 1H), 2.83 (dd, 1H), 2.75-2.58 (m, 3H), 2.42 (s, 3H), 1.20 (t, 3H); LC-MS (ESI) m/z 309.2 [M+H]⁺.

### Example 135: 3-(Cyclopentyl-N-(((3R)-2-methyl-1,2,3,4-tetrahydroisoquinolin-3-yl)methyl)propenamide (135)

The compound was prepared by general procedure i1 with Bix (61.6 mg, 0.349 mmol), 3-cyclopentylpropanoic acid (47.4 μL, 0.332 mmol), EDCI (86.8 mg, 0.454 mmol), HOBt (51.9 mg, 0.384 mmol) and DIPEA (87 μL, 0.524 mmol) in CH₂Cl₂ (4 mL), stirred at room temperature for 17 h. After workup, the crude was purified by Isco Combi-Flash Companion column chromatography (0-5% methanol in CH₂Cl₂) to give 135 (97.2 mg, 93%). ¹H NMR (400 MHz, DMSO-d₆) δ 7.77 (br t, 1H), 7.16-7.02 (m, 4H), 3.75 (d, 1H), 3.53 (d, 1H), 3.41-3.26 (m overlapped with H₂O, 1H), 3.03 (ddd, 1H), 2.80-2.59 (m, 3H), 2.35 (s, 3H), 2.10 (t, 2H), 1.79-1.64 (m, 4H), 1.63-1.43 (m, 5H), 1.12-0.98 (m, 2H); LC-MS (ESI) m/z 301.2 [M+H]⁺.

### Example 136: N-(((3R)-2-Methyl-1,2,3,4-tetrahydroisoquinolin-3-yl)methyl)-1H-indene-2-carboxamide (136)

The compound was prepared by general procedure i1 with Bix (62.7 mg, 0.356 mmol), 1H-indene-2-carboxylic acid (54.1 mg, 0.338 mmol), EDCI (88.4 mg, 0.462 mmol), HOBt (52.9 mg, 0.391 mmol) and DIPEA (90 μL, 0.534 mmol) in CH₂Cl₂ (4 mL), stirred at room temperature for 17 h. After workup, the crude was purified by Isco Combi-Flash Companion column chromatography (0-5% methanol in CH₂Cl₂) to give 136 (93.4 mg, 83%). ¹H NMR (400 MHz, DMSO-d₆) δ 8.13 (br t, 1H), 7.61-7.49 (m, 3H), 7.37-7.25 (m, 2H), 7.18-7.01 (m, 4H), 3.78 (d, 1H), 3.66 (d, 2H), 3.59 (d, 1H), 3.53 (dt, 1H), 3.16 (ddd, 1H), 2.96-2.79 (m, 2H), 2.69 (dd, 1H), 2.42 (s, 3H); LC-MS (ESI) m/z 319.2 [M+H]⁺.

### Example 137: 3-(4-Methylphenyl)-N-(((3R)-2-methyl-1,2,3,4-tetrahydroisoquinolin-3-yl)methyl)propanamide (137)

The compound was prepared by general procedure i1 with Bix (61.6 mg, 0.349 mmol), 3-(4-methylphenyl) propanoic acid (54.5 mg, 0.332 mmol), EDCI (86.8 mg, 0.454 mmol), HOBt (51.9 mg, 0.384 mmol) and DIPEA (87 μL, 0.524 mmol) in CH₂Cl₂ (4 mL), stirred at room temperature for 17 h. After workup, the crude was purified by Isco Combi-Flash Companion column chromatography (0-5% methanol in CH₂Cl₂) to give 137 (87 mg, 77%). ¹H NMR (400 MHz, DMSO-d₆) δ 7.78 (br t, 1H), 7.16-6.99 (m, 8H), 3.73 (d, 1H), 3.51 (d, 1H), 3.39-3.28 (m overlapped with H₂O, 1H), 3.07-2.96 (m, 1H), 2.77 (t, 2H), 2.71-2.53 (m, 3H), 2.38 (t, 2H), 2.31 (s, 3H), 2.24 (s, 3H); LC-MS (ESI) m/z 323.2 [M+H]⁺.

### Example 138: 3-(3-Methylphenyl)-N-(((3R)-2-methyl-1,2,3,4-tetrahydroisoquinolin-3-yl)methyl)propenamide (138)

The compound was prepared by general procedure i1 with Bix (61.6 mg, 0.349 mmol), 3-(3-methylphenyl) propanoic acid (54.5 mg, 0.332 mmol), EDCI (86.8 mg, 0.454 mmol), HOBt (51.9 mg, 0.384 mmol) and DIPEA (87 μL, 0.524 mmol) in CH₂Cl₂ (4 mL), stirred at room temperature for 17 h. After workup, the crude was purified by Isco Combi-Flash Companion column chromatography (0-5% methanol in CH₂Cl₂) to give 138 (82.6 mg, 73%). ¹H NMR (400 MHz, DMSO-d₆) δ 7.79 (br t, 1H), 7.22-7.07 (m, 3H), 7.07-6.94 (m, 5H), 3.73 (d, 1H), 3.51 (d, 1H), 3.39-3.27 (m overlapped with H₂O, 1H), 3.09-2.95 (m, 1H), 2.77 (t, 2H), 2.71-2.53 (m, 3H), 2.39 (t, 2H), 2.32 (s, 3H), 2.24 (s, 3H); LC-MS (ESI) m/z 323.2 [M+H]⁺.

### Example 139: 3-(3-Chlorophenyl)-N-(((3R)-2-methyl-1,2,3,4-tetrahydroisoquinolin-3-yl)methyl)propanamide (139)

The compound was prepared by general procedure i1 with Bix (61.6 mg, 0.349 mmol), 3-(3-chloro-phenyl) propanoic acid (61.3 mg, 0.332 mmol), EDCI (86.8 mg, 0.454 mmol), HOBt (51.9 mg, 0.384 mmol) and DIPEA (87 μL, 0.524 mmol) in CH₂Cl₂ (4 mL), stirred at room temperature for 17 h. After workup, the crude was purified by Isco Combi-Flash Companion column chromatography (0-5% methanol in CH₂Cl₂) to give 139 (100 mg, 84%). ¹H NMR (400 MHz, DMSO-d₆) δ 7.81 (br t, 1H), 7.33-7.26 (m, 2H), 7.25-7.21 (m, 1H), 7.18 (dt, 1H), 7.15-7.08 (m, 2H), 7.07-7.01 (m, 2H), 3.73 (d, 1H), 3.51 (d, 1H), 3.39-3.23 (m overlapped with H₂O, 1H), 3.02 (dt, 1H), 2.83 (t, 2H), 2.72-2.53 (m, 3H), 2.43 (t, 2H), 2.31 (s, 3H); LC-MS (ESI) m/z 343.1 [M+H]⁺.

### Example 140: 3-Cyclopentyl-N-(((3S)-2-methyl-1,2,3,4-tetrahydroisoquinolin-3-yl)methyl)propenamide (140)

The compound was prepared by general procedure i1 with Cix (68.7 mg, 0.39 mmol), 3-cyclopentylpropanoic acid (53 μL, 0.37 mmol), EDCI (96.8 mg, 0.507 mmol), HOBt (57.9 mg, 0.429 mmol) and DIPEA (97 μL, 0.585 mmol) in CH₂Cl₂ (4 mL), stirred at room temperature for 17 h. After workup, the crude was purified by Isco Combi-Flash Companion column chromatography (0-5% methanol in CH₂Cl₂) to give 140 (95.4 mg, 82%). ¹H NMR (400 MHz, DMSO-d₆) δ 7.77 (br t, 1H), 7.15-7.01 (m, 4H), 3.75 (d, 1H), 3.53 (d, 1H), 3.41-3.32 (m overlapped with H₂O, 1H), 3.04 (ddd, 1H), 2.80-2.57 (m, 3H), 2.35 (s, 3H), 2.10 (t, 2H) 1.80-1.64 (m, 4H), 1.63-1.39 (m, 5H), 1.15-0.98 (m, 2H); LC-MS (ESI) m/z 301.2 [M+H]⁺.

### Example 141: (2R)—N-(((3S)-2-methyl-1,2,3,4-tetrahydroisoquinolin-3-yl)methyl)-1,2,3,4-tetrahydronaphthalene-2-carboxamide (141)

The compound was prepared by general procedure i1 with Cix (68.7 mg, 0.39 mmol), (2R)-1,2,3,4-tetrahydronaphthalene-2-carboxylic acid (65.3 mg, 0.37 mmol), EDCI (96.8 mg, 0.507 mmol), HOBt (57.9 mg, 0.429 mmol) and DIPEA (97 μL, 0.585 mmol) in CH₂Cl₂ (4 mL), stirred at room temperature for 17 h. After workup, the crude was purified by Isco Combi-Flash Companion column chromatography (0-5% methanol in CH₂Cl₂) to give 141 (107 mg, 82%). [α]_{D}²⁴=−2.68° (c=1, MeOH); ¹H NMR (400 MHz, DMSO-d₆) δ 7.88 (br t, 1H), 7.17-7.01 (m, 8H), 3.77 (d, 1H), 3.55 (d, 1H), 3.38 (ddd, 1H), 3.11 (ddd, 1H), 2.90-2.62 (m, 7H), 2.62-2.52 (m, 1H), 2.37 (s, 3H), 2.00-1.88 (m, 1H), 1.77-1.59 (m, 1H); LC-MS (ESI) m/z 335.2 [M+H]⁺.

### Example 142: (2S)—N-(((3S)-2-Methyl-1,2,3,4-tetrahydroisoquinolin-3-yl)methyl)-1,2,3,4-tetrahydronaphthalene-2-carboxamide (142)

The compound was prepared by general procedure i1 with Cix (68.7 mg, 0.39 mmol), (2S)-1,2,3,4-tetrahydronaphthalene-2-carboxylic acid (65.3 mg, 0.37 mmol), EDCI (96.8 mg, 0.507 mmol), HOBt (57.9 mg, 0.429 mmol) and DIPEA (97 μL, 0.585 mmol) in CH₂Cl₂ (4 mL), stirred at room temperature for 17 h. After workup, the crude was purified by Isco Combi-Flash Companion column chromatography (0-5% methanol in CH₂Cl₂) to give 142 (106 mg, 81%). [α]_{D}²⁴=−0.58° (c=1, MeOH); ¹H NMR (400 MHz, DMSO-d₆) δ 7.89 (br t, 1H), 7.17-7.01 (m, 8H), 3.77 (d, 1H), 3.55 (d, 1H), 3.42 (ddd, 1H), 3.07 (ddd, 1H), 2.92-2.63 (m, 7H), 2.63-2.53 (m, 1H), 2.37 (s, 3H), 2.00-1.89 (m, 1H), 1.78-1.62 (m, 1H); LC-MS (ESI) m/z 335.2 [M+H]⁺.

### Example 143: N-(((3S)-2-Methyl-1,2,3,4-tetrahydroisoquinolin-3-yl)methyl)-2,3-dihydro-1H-indene-2-carboxamide (143)

The compound was prepared by general procedure i1 with Cix (755 mg, 4.28 mmol), 2-indanecarboxylic acid (625 mg, 3.86 mmol), 1-(3-dimethylaminopropyl)-3-ethylcarbodiimide hydrochloride (1.23 g, 6.42 mmol), 1-hydroxybenzotriazole hydrate (868 mg, 6.42 mmol) and N,N-diisopropylethylamine (2.60 mL, 15 mmol) in dichloromethane (22 mL). The crude was purified by flash column chromatography (SiO₂, 3% methanol in dichloromethane) to give 143 as a white solid (1.167 g, 95%). [α]_{D}²⁴=−42.46° (c=1, MeOH); ¹H NMR (600 MHz, DMSO-d₆) δ 7.95 (br t, 1H), 7.20-7.16 (m, 2H), 7.14-7.07 (m, 5H), 7.06-7.03 (m, 1H), 3.76 (d, 1H), 3.55 (d, 1H), 3.40 (dt, 1H), 3.22 (quin., 1H), 3.12-3.06 (m, 1H), 3.06-3.01 (m, 4H), 2.77 (dd, 1H), 2.74-2.69 (m, 1H), 2.66 (dd, 1H), 2.36 (s, 3H); LC-MS (ESI) m/z 321.2 [M+H]⁺.

### Example 144: N-(((3S)-2-methyl-1,2,3,4-tetrahydroisoquinolin-3-yl)methyl)-1H-indene-2-carboxamide (144)

The compound was prepared by general procedure i1 with Cix (40.9 mg, 0.232 mmol), 1H-indene-2-carboxylic acid (35.3 mg, 0.22 mmol), EDCI (57.6 mg, 0.302 mmol), HOBt (34.5 mg, 0.255 mmol) and DIPEA (60 μL, 0.348 mmol) in CH₂Cl₂ (4 mL), stirred at room temperature for 17 h. After workup, the crude was purified by Isco Combi-Flash Companion column chromatography (0-5% methanol in CH₂Cl₂) to give 144 (52.6 mg, 71%). ¹H NMR (400 MHz, DMSO-d₆) δ 8.13 (br t, 1H), 7.60-7.49 (m, 3H), 7.39-7.24 (m, 2H), 7.18-7.02 (m, 4H), 3.79 (d, 1H), 3.66 (d, 2H), 3.60 (d, 1H), 3.52 (ddd, 1H), 3.16 (ddd, 1H), 2.96-2.79 (m, 2H), 2.69 (dd, 1H), 2.42 (s, 3H); LC-MS (ESI) m/z 319.1 [M+H]⁺.

### Example 145: 3-(4-Methylphenyl)-N-(((3S)-2-methyl-1,2,3,4-tetrahydroisoquinolin-3-yl)methyl)propanamide (145)

The compound was prepared by general procedure i1 with Cix (68.7 mg, 0.39 mmol), 3-(4-methylphenyl) propanoic acid (60.8 mg, 0.37 mmol), EDCI (96.8 mg, 0.507 mmol), HOBt (57.9 mg, 0.429 mmol) and DIPEA (97 μL, 0.585 mmol) in CH₂Cl₂ (4 mL), stirred at room temperature for 17 h. After workup, the crude was purified by Isco Combi-Flash Companion column chromatography (0-5% methanol in CH₂Cl₂) to give 145 (90.9 mg, 73%). ¹H NMR (400 MHz, DMSO-d₆) δ 7.78 (br t, 1H), 7.17-7.00 (m, 8H), 3.73 (d, 1H), 3.51 (d, 1H), 3.39-3.28 (m overlapped with H₂O, 1H), 3.02 (dt, 1H), 2.77 (t, 2H), 2.71-2.54 (m, 3H), 2.38 (t, 2H), 2.31 (s, 3H), 2.24 (s, 3H); LC-MS (ESI) m/z 323.2 [M+H]⁺.

### Example 146: 3-(3-Methylphenyl)-N-(((3S)-2-methyl-1,2,3,4-tetrahydroisoquinolin-3-yl)methyl)propenamide (146)

The compound was prepared by general procedure i1 with Cix (68.7 mg, 0.39 mmol), 3-(3-methylphenyl) propanoic acid (60.8 mg, 0.37 mmol), EDCI (96.8 mg, 0.507 mmol), HOBt (57.9 mg, 0.429 mmol) and DIPEA (97 μL, 0.585 mmol) in CH₂Cl₂ (4 mL), stirred at room temperature for 17 h. After workup, the crude was purified by Isco Combi-Flash Companion column chromatography (0-5% methanol in CH₂Cl₂) to give 146 (86.1 mg, 69%). ¹H NMR (400 MHz, DMSO-d₆) δ 7.79 (br t, 1H), 7.20-7.07 (m, 3H), 7.07-6.93 (m, 5H), 3.73 (d, 1H), 3.51 (d, 1H), 3.37-3.29 (m overlapped with H₂O, 1H), 3.02 (ddd, 1H), 2.77 (t, 2H), 2.71-2.52 (m, 3H), 2.39 (t, 2H), 2.32 (s, 3H), 2.24 (s, 3H); LC-MS (ESI) m/z 323.2 [M+H]⁺.

### Example 147: 3-(3-Chlorophenyl)-N-(((3S)-2-methyl-1,2,3,4-tetrahydroisoquinolin-3-yl)methyl)propanamide (147)

The compound was prepared by general procedure i1 with Cix (68.7 mg, 0.39 mmol), 3-(3-chlorophenyl) propanoic acid (68.4 mg, 0.37 mmol), EDCI (96.8 mg, 0.507 mmol), HOBt (57.9 mg, 0.429 mmol) and DIPEA (97 μL, 0.585 mmol) in CH₂Cl₂ (4 mL), stirred at room temperature for 17 h. After workup, the crude was purified by Isco Combi-Flash Companion column chromatography (0-5% methanol in CH₂Cl₂) to give 147 (105 mg, 79%). ¹H NMR (400 MHz, DMSO-d₆) δ 7.81 (br t, 1H), 7.33-7.26 (m, 2H), 7.23 (ddd, 1H), 7.18 (dt, 1H), 7.15-7.07 (m, 2H), 7.07-7.01 (m, 2H), 3.73 (d, 1H), 3.51 (d, 1H), 3.39-3.25 (m overlapped with H₂O, 1H), 3.02 (ddd, 1H), 2.83 (t, 2H), 2.73-2.53 (m, 3H), 2.43 (t, 2H), 2.31 (s, 3H); LC-MS (ESI) m/z 323.2 [M+H]⁺.

### Example 148: N-((2,5-Dimethyl-1,2,3,4-tetrahydroisoquinolin-3-yl)methyl)-3-(3-fluorophenyl)propenamide (148)

The compound was prepared by general procedure i1 with Zix (57.5 mg, 0.3 mmol), 3-(3-fluorophenyl) propanoic acid (48.3 mg, 0.285 mmol), EDCI (75.1 mg, 0.393 mmol), HOBt (44.9 mg, 0.332 mmol) and DIPEA (75 μL, 0.453 mmol) in CH₂Cl₂ (4 mL), stirred at room temperature for 17 h. After workup, the crude was purified by Isco Combi-Flash Companion column chromatography (0-5% methanol in CH₂Cl₂) to give 148 (81.2 mg, 79%). ¹H NMR (400 MHz, DMSO-d₆) δ 7.83 (br t, 1H), 7.34-7.24 (m, 1H), 7.11-6.94 (m, 5H), 6.87 (d, 1H), 3.72 (d, 1H), 3.50 (d, 1H), 3.41-3.34 (m, 1H), 3.08 (ddd, 1H), 2.85 (t, 2H), 2.68-2.54 (m, 2H), 2.44 (t, 2H), 2.42-2.36 (m, 1H), 2.29 (s, 3H), 2.14 (s, 3H); LC-MS (ESI) m/z 341.2 [M+H]⁺.

### Example 149: N-((2,5-Dimethyl-1,2,3,4-tetrahydroisoquinolin-3-yl)methyl)-3-phenylpropanamide (149)

The compound was prepared by general procedure i1 with Zix (57.5 mg, 0.3 mmol), 3-phenylpropanoic acid (43.1 mg, 0.287 mmol), EDCI (75.1 mg, 0.393 mmol), HOBt (44.9 mg, 0.332 mmol) and DIPEA (75 μL, 0.453 mmol) in CH₂Cl₂ (4 mL), stirred at room temperature for 17 h. After workup, the crude was purified by Isco Combi-Flash Companion column chromatography (0-5% methanol in CH₂Cl₂) to give 149 (75.1 mg, 77%). ¹H NMR (400 MHz, DMSO-d₆) δ 7.82 (br t, 1H), 7.32-7.12 (m, 5H), 7.07-6.95 (m, 2H), 6.87 (d, 1H), 3.72 (d, 1H), 3.50 (d, 1H), 3.42-3.33 (m, 1H), 3.09 (ddd, 1H), 2.82 (t, 2H), 2.71-2.53 (m, 2H), 2.48-2.37 (m, 3H), 2.30 (s, 3H), 2.14 (s, 3H); LC-MS (ESI) m/z 323.2 [M+H]⁺.

### Example 150: 3-(Cyclohexyl-N-((2,5-dimethyl-1,2,3,4-tetrahydroisoquinolin-3-yl)methyl)propenamide (150)

The compound was prepared by general procedure i1 with Zix (57.5 mg, 0.3 mmol), 3-cyclohexylpropanoic acid (47 μL, 0.272 mmol), EDCI (75.1 mg, 0.393 mmol), HOBt (44.9 mg, 0.332 mmol) and DIPEA (75 μL, 0.453 mmol) in CH₂Cl₂ (4 mL), stirred at room temperature for 17 h. After workup, the crude was purified by Isco Combi-Flash Companion column chromatography (0-5% methanol in CH₂Cl₂) to give 150 (78 mg, 79%). ¹H NMR (400 MHz, DMSO-d₆) δ 7.77 (br t, 1H), 7.11-6.94 (m, 2H), 6.87 (d, 1H), 3.73 (d, 1H), 3.51 (d, 1H), 3.41-3.34 (m, 1H), 3.10 (ddd, 1H), 2.74-2.53 (m, 2H), 2.49-2.40 (m, 1H), 2.33 (s, 3H), 2.17 (s, 3H), 2.11 (t, 2H), 1.77-1.53 (m, 5H), 1.40 (q, 2H), 1.28-1.01 (m, 4H), 0.92-0.76 (m, 2H); LC-MS (ESI) m/z 329.3 [M+H]⁺.

### Example 151: N-((2,5-Dimethyl-1,2,3,4-tetrahydroisoquinolin-3-yl)methyl)-2,3-dihydro-1H-indene-2-carboxamide (151)

The compound was prepared by general procedure i1 with Zix (57.5 mg, 0.3 mmol), 2,3-dihydro-1H-indene-2-carboxylic acid (46.2 mg, 0.285 mmol), EDCI (75.1 mg, 0.393 mmol), HOBt (44.9 mg, 0.332 mmol) and DIPEA (75 μL, 0.453 mmol) in CH₂Cl₂ (4 mL), stirred at room temperature for 17 h. After workup, the crude was purified by Isco Combi-Flash Companion column chromatography (0-5% methanol in CH₂Cl₂) to give 151 (79.4 mg, 79%). ¹H NMR (400 MHz, DMSO-d₆) δ 7.95 (br t, 1H), 7.24-7.16 (m, 2H), 7.16-7.08 (m, 2H), 7.06-6.95 (m, 2H), 6.88 (d, 1H), 3.76 (d, 1H), 3.54 (d, 1H), 3.41 (ddd, 1H), 3.29-3.12 (m, 2H), 3.07 (d, 2H), 3.05 (d, 2H), 2.79-2.58 (m, 2H), 2.54-2.45 (m overlapped with DMSO-d₆, 1H), 2.35 (s, 3H), 2.17 (s, 3H); LC-MS (ESI) m/z 335.2 [M+H]⁺.

### Example 152: N-(((3S)-2,5-dimethyl-1,2,3,4-tetrahydroisoquinolin-3-yl)methyl)-2,3-dihydro-1H-indene-2-carboxamide (152)

The compound was prepared by general procedure i1 with Eix (67 mg, 0.352 mmol), 2,3-dihydro-1H-indene-2-carboxylic acid (54 mg, 0.335 mmol), EDCI (87.5 mg, 0.458 mmol), HOBt (52.3 mg, 0.387 mmol) and DIPEA (87 μL, 0.528 mmol) in CH₂Cl₂ (4 mL), stirred at room temperature for 17 h. After workup, the crude was purified by Isco Combi-Flash Companion column chromatography (0-5% methanol in CH₂Cl₂) to give 152 (86.7 mg, 74%). ¹H NMR (400 MHz, DMSO-d₆) δ 7.95 (br t, 1H), 7.23-7.16 (m, 2H), 7.16-7.09 (m, 2H), 7.06-6.97 (m, 2H), 6.88 (d, 1H), 3.76 (d, 1H), 3.53 (d, 1H), 3.41 (ddd, 1H), 3.30-3.13 (m, 2H), 3.07 (d, 2H), 3.05 (d, 2H), 2.77-2.67 (m, 1H), 2.63 (dd, 1H), 2.54-2.44 (m overlapped with DMSO-d₆, 1H), 2.35 (s, 3H), 2.17 (s, 3H); LC-MS (ESI) m/z 335.2 [M+H]⁺.

### Example 153: N-((2,6-Dimethyl-1,2,3,4-tetrahydroisoquinolin-3-yl)methyl)-3-(3-fluorophenyl)propanamide (153)

The compound was prepared by general procedure i1 with Kix (45 mg, 0.236 mmol), 3-(3-fluorophenyl) propionic acid (38 mg, 0.225 mmol), 1-(3-dimethylaminopropyl)-3-ethylcarbodiimide hydrochloride (68 mg, 0.355 mmol), 1-hydroxybenzotriazole hydrate (48 mg, 0.355 mmol) and N,N-diisopropylethylamine (144 μL, 0.828 mmol) in dichloromethane (4.7 mL). The crude was purified by flash column chromatography (SiO₂, 2% methanol in dichloromethane) to give 153 as a yellow syrup (39 mg, 51%). ¹H NMR (600 MHz, CDCl₃) δ 7.21 (td, 1H), 7.00-6.94 (m, 2H), 6.93-6.84 (m, 4H), 6.18 (br s, 1H), 3.79 (d, 1H), 3.67 (d, 1H), 3.45 (dt, 1H), 3.31 (ddd, 1H), 2.96 (t, 2H), 2.86-2.81 (m, 1H), 2.70 (dd, 1H), 2.62 (dd, 1H), 2.50 (t, 2H), 2.33 (s, 3H), 2.29 (s, 3H); LC-MS (ESI) m/z 341.2 [M+H]⁺.

### Example 154: N-((2,6-Dimethyl-1,2,3,4-tetrahydroisoquinolin-3-yl)methyl)-2,3-dihydro-1H-indene-2-carboxamide (154)

The compound was prepared by general procedure i1 with Kix (34.5 mg, 0.181 mmol), 2-indanecarboxylic acid (28 mg, 0.172 mmol), 1-(3-dimethylaminopropyl)-3-ethylcarbodiimide hydrochloride (52 mg, 0.272 mmol), 1-hydroxybenzotriazole hydrate (37 mg, 0.272 mmol) and N,N-diisopropylethylamine (110 μL, 0.635 mmol) in dichloromethane (3.6 mL). The crude was purified by flash column chromatography (SiO₂, 3-4% methanol in dichloromethane) to give 154 as a yellow syrup (23 mg, 40%). ¹H NMR (600 MHz, CDCl₃) δ 7.24-7.20 (m, 2H), 7.19-7.15 (m, 2H), 7.00 (d, 1H), 6.96 (d, 1H), 6.94 (s, 1H), 6.35 (br s, 1H), 3.86 (d, 1H), 3.73 (d, 1H), 3.53 (dt, 1H), 3.43 (ddd, 1H), 3.30-3.14 (m, 5H), 2.96-2.90 (m, 1H), 2.80 (dd, 1H), 2.74 (dd, 1H), 2.42 (s, 3H), 2.32 (s, 3H); LC-MS (ESI) m/z 335.2 [M+H]⁺.

### Example 155: N-((2,6-Dimethyl-1,2,3,4-tetrahydroisoquinolin-3-yl)methyl)-1,2,3,4-tetrahydronaphthalene-2-carboxamide (155)

The compound was prepared by general procedure i1 with Kix (34.5 mg, 0.181 mmol), 1,2,3,4-tetrahydronaphthalene-2-carboxylic acid (30 mg, 0.172 mmol), 1-(3-dimethylaminopropyl)-3-ethylcarbodiimide hydrochloride (52 mg, 0.272 mmol), 1-hydroxybenzotriazole hydrate (37 mg, 0.272 mmol) and N,N-diisopropylethylamine (110 μL, 0.635 mmol) in dichloromethane (3.6 mL). The crude was purified by flash column chromatography (SiO₂, 2-4% methanol in dichloromethane) to give 155 as a colorless syrup (36 mg, 60%). ¹H NMR (600 MHz, CDCl₃) δ 7.15-7.02 (m, 4H), 7.02-6.88 (m, 3H), 6.34 (br s, 1H), 3.83 (dd, 1H), 3.69 (d, 1H), 3.51 (dtd, 1H), 3.44-3.36 (m, 1H), 3.03 (ddd, 1H), 2.96-2.68 (m, 6H), 2.58-2.51 (m, 1H), 2.39 (d, 3H), 2.30 (s, 3H), 2.12-2.06 (m, 1H), 1.95-1.85 (m, 1H); LC-MS (ESI) m/z 349.3 [M+H]⁺.

### Example 156: N-((2,6-Dimethyl-1,2,3,4-tetrahydroisoquinolin-3-yl)methyl)-5-fluoro-2,3-dihydro-1H-indene-2-carboxamide (156)

The compound was prepared by general procedure i1 with Kix (34.5 mg, 0.181 mmol), 5-fluoro-2,3-dihydro-1H-indene-2-carboxylic acid (31 mg, 0.172 mmol), 1-(3-dimethylaminopropyl)-3-ethylcarbodiimide hydrochloride (52 mg, 0.272 mmol), 1-hydroxybenzotriazole hydrate (37 mg, 0.272 mmol) and N,N-diisopropylethylamine (110 μL, 0.635 mmol) in dichloromethane (3.6 mL). The crude was purified by flash column chromatography (SiO₂, 2-4% methanol in dichloromethane) to give 156 as a colorless syrup (32 mg, 53%). ¹H NMR (600 MHz, CDCl₃) δ 7.12-7.08 (m, 1H), 6.97 (d, 1H), 6.93 (d, 1H), 6.91 (s, 1H), 6.87 (d, 1H), 6.83 (td, 1H), 6.29 (br s, 1H), 3.83 (d, 1H), 3.70 (d, 1H), 3.51 (dt, 1H), 3.38 (dt, 1H), 3.27-3.06 (m, 5H), 2.93-2.87 (m, 1H), 2.82-2.68 (m, 2H), 2.40 (s, 3H), 2.30 (s, 3H); LC-MS (ESI) m/z 353.2 [M+H]⁺.

### Example 157: (3S)—N-((2,6-Dimethyl-1,2,3,4-tetrahydroisoquinolin-3-yl)methyl)-2-methyl-1,2,3,4-tetrahydroisoquinoline-3-carboxamide (157)

The compound was prepared by general procedure i1 with Kix (45 mg, 0.236 mmol), (3S)-2-methyl-1,2,3,4-tetrahydroisoquinoline-3-carboxylic acid (51 mg, 0.225 mmol), 1-(3-dimethylamino-propyl)-3-ethylcarbodiimide hydrochloride (68 mg, 0.355 mmol), 1-hydroxybenzotriazole hydrate (48 mg, 0.355 mmol) and N,N-diisopropylethylamine (144 μL, 0.828 mmol) in dichloromethane (4.7 mL). The crude was purified by flash column chromatography (SiO₂, 3-6% methanol in dichloromethane) to give 157 as a yellow syrup (26 mg, 32%). ¹H NMR (600 MHz, Acetone-d₆) δ 7.49 (br s, 1H), 7.17-7.09 (m, 3H), 7.09-7.05 (m, 1H), 6.95-6.89 (m, 2H), 6.86 (d, 1H), 3.89 (dd, 1H), 3.76 (dd, 1H), 3.60 (d, 1H), 3.53 (d, 1H), 3.41 (ddt, 1H), 3.28 (ddt, 1H), 3.21 (dd, 1H), 3.08-3.01 (m overlapped with H₂O, 1H), 2.96 (dd, 1H), 2.77-2.70 (m, 1H), 2.70-2.52 (m, 2H), 2.39 (d, 3H), 2.35 (d, 3H), 2.25 (s, 3H); LC-MS (ESI) m/z 364.3 [M+H]⁺.

### Example 158: N-((2,6-Dimethyl-1,2,3,4-tetrahydroisoquinolin-3-yl)methyl)-1H-indene-2-carboxamide (158)

The compound was prepared by general procedure i1 with Kix (34.5 mg, 0.181 mmol), 1H-indene-2-carboxylic acid (29 mg, 0.172 mmol), 1-(3-dimethylaminopropyl)-3-ethylcarbodiimide hydrochloride (52 mg, 0.272 mmol), 1-hydroxybenzotriazole hydrate (36.7 mg, 0.272 mmol) and N,N-diisopropylethylamine (110 μL, 0.635 mmol) in dichloromethane (3.6 mL). The crude was purified by flash column chromatography (SiO₂, 2-4% methanol in dichloromethane) to give 158 as a beige solid (27 mg, 47%). ¹H NMR (600 MHz, CDCl₃) δ 7.49-7.44 (m, 3H), 7.33-7.27 (m, 2H), 7.00-6.93 (m, 2H), 6.92 (s, 1H), 6.75 (br s, 1H), 3.88 (d, 1H), 3.74 (d, 1H), 3.70-3.59 (m, 3H), 3.55-3.48 (m, 1H), 3.04-2.99 (m, 1H), 2.88-2.75 (m, 2H), 2.46 (s, 3H), 2.27 (s, 3H); LC-MS (ESI) m/z 333.2 [M+H]⁺.

### Example 159: 3,4-Dichloro-N-(((3R)-2,6-dimethyl-1,2,3,4-tetrahydroisoquinolin-3-yl)methyl)benzamide (159)

The compound was prepared by general procedure i1 with Lix (67.5 mg, 0.355 mmol), 3,4-dichlorobenzoic acid (64 mg, 0.337 mmol), 1-(3-dimethylaminopropyl)-3-ethylcarbodiimide hydrochloride (102 mg, 0.532 mmol), 1-hydroxybenzotriazole hydrate (72 mg, 0.532 mmol) and N,N-diisopropylethylamine (220 μL, 1.24 mmol) in dichloromethane (7 mL). The crude was purified by flash column chromatography (SiO₂, 3% methanol in dichloromethane) to give 159 as a yellow syrup (95 mg, 78%). ¹H NMR (600 MHz, DMSO-d₆) δ 8.63 (br t, 1H), 8.05 (d, 1H), 7.82 (dd, 1H), 7.75 (d, 1H), 6.95-6.91 (m, 2H), 6.90 (s, 1H), 3.73 (d, 1H), 3.60-3.51 (m, 2H), 3.18 (dt, 1H), 2.97-2.89 (m, 1H), 2.79 (dd, 1H), 2.64 (dd, 1H), 2.40 (s, 3H), 2.23 (s, 3H); LC-MS (ESI) m/z 363.1 [M+H]⁺.

### Example 160: (3S)—N-(((3R)-2,6-Dimethyl-1,2,3,4-tetrahydroisoquinolin-3-yl)methyl)-2-methyl-1,2,3,4-tetrahydroisoquinoline-3-carboxamide (160)

The compound was prepared by general procedure i1 with Lix (67.5 mg, 0.355 mmol), (35)-2-methyl-1,2,3,4-tetrahydroisoquinoline-3-carboxylic acid (77 mg, 0.337 mmol), 1-(3-dimethylaminopropyl)-3-ethylcarbodiimide hydrochloride (102 mg, 0.532 mmol), 1-hydroxybenzotriazole hydrate (72 mg, 0.532 mmol) and N,N-diisopropylethylamine (220 μL, 1.24 mmol) in dichloromethane (7 mL). The crude was purified by flash column chromatography (SiO₂, 3-6% methanol in dichloromethane) to give 160 as a brown syrup (41 mg, 33%). ¹H NMR (600 MHz, DMSO-d₆) δ 7.90 (br t, 1H), 7.14-7.08 (m, 3H), 7.08-7.03 (m, 1H), 6.94-6.88 (m, 2H), 6.86 (s, 1H), 3.87 (d, 1H), 3.70 (d, 1H), 3.55-3.46 (m, 2H), 3.38-3.34 (m, 1H), 3.16 (dd, 1H), 3.09-3.04 (m, 1H), 2.94 (dd, 1H), 2.85 (dd, 1H), 2.75-2.68 (m, 1H), 2.65 (dd, 1H), 2.56-2.51 (m, 1H), 2.32 (s, 3H), 2.31 (s, 3H), 2.23 (s, 3H); LC-MS (ESI) m/z 364.2 [M+H]⁺.

### Example 161: N-(((3R)-2,6-Dimethyl-1,2,3,4-tetrahydroisoquinolin-3-yl)methyl) naphthalene-2-carboxamide (161)

The compound was prepared by general procedure i1 with Lix (67.5 mg, 0.355 mmol), naphthalene-2-carboxylic acid (58 mg, 0.337 mmol), 1-(3-dimethylaminopropyl)-3-ethylcarbodiimide hydrochloride (102 mg, 0.532 mmol), 1-hydroxybenzotriazole hydrate (72 mg, 0.532 mmol) and N,N-diisopropylethylamine (220 μL, 1.24 mmol) in dichloromethane (7 mL). The crude was purified by flash column chromatography (SiO₂, 3-6% methanol in dichloromethane) to give 161 as a white solid (81 mg, 70%). ¹H NMR (600 MHz, DMSO-d₆) δ 8.60 (br t, 1H), 8.43 (s, 1H), 8.04-8.01 (m, 1H), 7.98 (t, 2H), 7.93 (dd, 1H), 7.63-7.55 (m, 2H), 6.97-6.91 (m, 3H), 3.76 (d, 1H), 3.65-3.55 (m, 2H), 3.29-3.23 (m, 1H), 3.03-2.94 (m, 1H), 2.83 (dd, 1H), 2.70 (dd, 1H), 2.45 (s, 3H), 2.23 (s, 3H); LC-MS (ESI) m/z 345.1 [M+H]⁺.

### Example 162: (2R)—N-(((3R)-2,6-Dimethyl-1,2,3,4-tetrahydroisoquinolin-3-yl)methyl)-1,2,3,4-tetrahydronaphthalene-2-carboxamide (162)

The compound was prepared by general procedure i1 with Lix (67.5 mg, 0.355 mmol), (2R)-1,2,3,4-tetrahydronaphthalene-2-carboxylic acid (59 mg, 0.337 mmol), 1-(3-dimethylaminopropyl)-3-ethylcarbodiimide hydrochloride (102 mg, 0.532 mmol), 1-hydroxybenzotriazole hydrate (72 mg, 0.532 mmol) and N,N-diisopropylethylamine (220 μL, 1.24 mmol) in dichloromethane (7 mL). The crude was purified by flash column chromatography (SiO₂, 3-6% methanol in dichloromethane) to give 162 as a white solid (79 mg, 67%). [α]_{D}²⁴=+3.59° (c=1, MeOH); ¹H NMR (600 MHz, DMSO-d₆) δ 7.87 (br t, 1H), 7.08-7.04 (m, 4H), 6.94-6.91 (m, 2H), 6.89 (s, 1H), 3.72 (d, 1H), 3.51 (d, 1H), 3.37 (dt, 1H), 3.08 (dt, 1H), 2.86-2.68 (m, 6H), 2.65-2.58 (m, 1H), 2.58-2.52 (m, 1H), 2.36 (s, 3H), 2.24 (s, 3H), 1.97-1.90 (m, 1H), 1.72-1.64 (m, 1H); LC-MS (ESI) m/z 349.2 [M+H]⁺.

### Example 163: (2S)—N-(((3R)-2,6-Dimethyl-1,2,3,4-tetrahydroisoquinolin-3-yl)methyl)-1,2,3,4-tetrahydronaphthalene-2-carboxamide (163)

The compound was prepared by general procedure i1 with Lix (67.5 mg, 0.355 mmol), (2S)-1,2,3,4-tetrahydronaphthalene-2-carboxylic acid (59 mg, 0.337 mmol), 1-(3-dimethylaminopropyl)-3-ethylcarbodiimide hydrochloride (102 mg, 0.532 mmol), 1-hydroxybenzotriazole hydrate (72 mg, 0.532 mmol) and N,N-diisopropylethylamine (220 μL, 1.24 mmol) in dichloromethane (7 mL). The crude was purified by flash column chromatography (SiO₂, 3-6% methanol in dichloromethane) to give 163 as a white solid (73 mg, 62%). [α]_{D}²⁴=+10.16° (c=1, MeOH); ¹H NMR (600 MHz, DMSO-d₆) δ 7.87 (br t, 1H), 7.08-7.03 (m, 4H), 6.94-6.91 (m, 2H), 6.90 (s, 1H), 3.72 (d, 1H), 3.51 (d, 1H), 3.40 (dt, 1H), 3.05 (dt, 1H), 2.88-2.67 (m, 6H), 2.66-2.59 (m, 1H), 2.58-2.52 (m, 1H), 2.36 (s, 3H), 2.24 (s, 3H), 1.96-1.90 (m, 1H), 1.72-1.63 (m, 1H); LC-MS (ESI) m/z 349.2 [M+H]⁺.

### Example 164: N-(((3S)-2,6-Dimethyl-1,2,3,4-tetrahydroisoquinolin-3-yl)methyl)-4-(ethylsulfanyl)benzamide (164)

The compound was prepared by general procedure i1 with bix (27.5 mg, 0.145 mmol), 4-(ethylsulfanyl)benzoic acid (25 mg, 0.137 mmol), 1-(3-dimethylaminopropyl)-3-ethylcarbodiimide hydrochloride (41.5 mg, 0.217 mmol), 1-hydroxybenzotriazole hydrate (29 mg, 0.217 mmol) and N,N-diisopropylethylamine (88 μL, 0.506 mmol) in dichloromethane (2.9 mL). The crude was purified by flash column chromatography (SiO₂, 3-6% methanol in dichloromethane) to give 164 as a white solid (31 mg, 64%). ¹H NMR (600 MHz, CDCl₃) δ 7.66 (d, 2H), 7.28 (d, 2H), 7.01-6.93 (m, 2H), 6.91 (s, 1H), 6.87 (br s, 1H), 3.86 (d, 1H), 3.72 (d, 1H), 3.66 (dt, 1H), 3.51 (ddd, 1H), 3.04-2.95 (m, 3H), 2.82 (dd, 1H), 2.77 (dd, 1H), 2.44 (s, 3H), 2.28 (s, 3H), 1.34 (t, 3H); LC-MS (ESI) m/z 355.2 [M+H]⁺.

### Example 165: 3,4-Dichloro-N-(((3S)-2,6-dimethyl-1,2,3,4-tetrahydroisoquinolin-3-yl)methyl)benzamide (165)

The compound was prepared by general procedure i2 with bix (27.5 mg, 0.145 mmol), 3,4-dichlorobenzoyl chloride (26 mg, 0.137 mmol), and triethylamine (20 μL, 0.145 mmol) in dichloromethane (2.9 mL). The crude was purified by flash column chromatography (SiO₂, 2-4% methanol in dichloromethane) to give 165 as a colorless syrup (34 mg, 68%). ¹H NMR (600 MHz, CDCl₃) δ 7.78 (d, 1H), 7.54 (dd, 1H), 7.47 (d, 1H), 6.98 (d, 1H), 6.95 (d, 1H), 6.91 (s, 1H), 6.86 (br s, 1H), 3.85 (d, 1H), 3.73 (d, 1H), 3.65 (dt, 1H), 3.50 (ddd, 1H), 3.05-2.99 (m, 1H), 2.84 (dd, 1H), 2.75 (dd, 1H), 2.44 (s, 3H), 2.28 (s, 3H); LC-MS (ESI) m/z 363.1 [M+H]⁺.

### Example 166: N-(((3S)-2,6-Dimethyl-1,2,3,4-tetrahydroisoquinolin-3-yl)methyl)-3-(3-fluorophenyl)propanamide (166)

The compound was prepared by general procedure i1 with bix (36.8 mg, 0.193 mmol), 3-(3-fluorophenyl) propanoic acid (31 mg, 0.183 mmol), 1-(3-dimethylaminopropyl)-3-ethylcarbodiimide hydrochloride (56 mg, 0.29 mmol), 1-hydroxybenzotriazole hydrate (39 mg, 0.29 mmol) and N,N-diisopropylethylamine (120 μL, 0.676 mmol) in dichloromethane (3.9 mL). The crude was purified by flash column chromatography (SiO₂, 3% methanol in dichloromethane) to give 166 as a white solid (27 mg, 43%). ¹H NMR (600 MHz, CDCl₃) δ 7.21 (td, 1H), 7.00-6.93 (m, 2H), 6.93-6.84 (m, 4H), 6.11 (br s, 1H), 3.77 (d, 1H), 3.65 (d, 1H), 3.44 (dt, 1H), 3.30 (ddd, 1H), 2.96 (t, 2H), 2.83-2.78 (m, 1H), 2.69 (dd, 1H), 2.61 (dd, 1H), 2.49 (t, 2H), 2.31 (s, 3H), 2.29 (s, 3H); LC-MS (ESI) m/z 341.2 [M+H]⁺.

### Example 167: N-(((3S)-2,6-Dimethyl-1,2,3,4-tetrahydroisoquinolin-3-yl)methyl)-3-phenylpropanamide (167)

The compound was prepared by general procedure i1 with bix (133 mg, 0.699 mmol), 3-phenylpropionic acid (100 mg, 0.664 mmol), 1-(3-dimethylaminopropyl)-3-ethylcarbodiimide hydrochloride (201 mg, 1.05 mmol), 1-hydroxybenzotriazole hydrate (142 mg, 1.05 mmol) and N,N-diisopropylethylamine (430 μL, 2.45 mmol) in dichloromethane (2.9 mL). The crude was purified by flash column chromatography (SiO₂, 3-4% methanol in dichloromethane) to give 167 as a white solid (113 mg, 50%). [α]_{D}²⁴=−36.60° (c=1, MeOH); ¹H NMR (600 MHz, CDCl₃) δ 7.29-7.25 (m overlapped with CDCl₃, 2H), 7.22-7.16 (m, 3H), 6.96 (d, 1H), 6.91 (d, 1H), 6.88 (s, 1H), 6.06 (br s, 1H), 3.77 (d, 1H), 3.64 (d, 1H), 3.43 (dt, 1H), 3.30 (ddd, 1H), 2.97 (t, 2H), 2.83-2.77 (m, 1H), 2.68 (dd, 1H), 2.61 (dd, 1H), 2.53-2.48 (m, 2H), 2.31 (s, 3H), 2.30 (s, 3H); LC-MS (ESI) m/z 323.2 [M+H]⁺.

### Example 168: 3-(Cyclohexyl-N-(((3S)-2,6-dimethyl-1,2,3,4-tetrahydroisoquinolin-3-yl)methyl)propanamide (168)

The compound was prepared by general procedure i1 with bix (36.8 mg, 0.193 mmol), 3-cyclohexylpropanoic acid (29 μL, 0.183 mmol), 1-(3-dimethylaminopropyl)-3-ethylcarbodiimide hydrochloride (56 mg, 0.29 mmol), 1-hydroxybenzotriazole hydrate (39 mg, 0.29 mmol) and N,N-diisopropylethylamine (120 μL, 0.676 mmol) in dichloromethane (3.9 mL). The crude was purified by flash column chromatography (SiO₂, 3-4% methanol in dichloromethane) to give 168 as a white solid (29 mg, 48%). ¹H NMR (600 MHz, CDCl₃) δ 6.99-6.89 (m, 3H), 6.32 (br s, 1H), 3.85 (d, 1H), 3.72 (d, 1H), 3.49 (dt, 1H), 3.36 (ddd, 1H), 2.95-2.89 (m, 1H), 2.79-2.69 (m, 2H), 2.41 (s, 3H), 2.29 (s, 3H), 2.22-2.17 (m, 2H), 1.73-1.65 (m, 4H), 1.65-1.59 (m, 1H), 1.55-1.48 (m, 2H), 1.27-1.08 (m, 4H), 0.93-0.84 (m, 2H); LC-MS (ESI) m/z 329.3 [M+H]⁺.

### Example 169: N-(((3S)-2,6-Dimethyl-1,2,3,4-tetrahydroisoquinolin-3-yl)methyl)-2,3-dihydro-1H-indene-2-carboxamide (169)

The compound was prepared by general procedure i1 with bix (27.5 mg, 0.145 mmol), 2-indanecarboxylic acid (22.3 mg, 0.137 mmol), 1-(3-dimethylaminopropyl)-3-ethylcarbodiimide hydrochloride (41.5 mg, 0.217 mmol), 1-hydroxybenzotriazole hydrate (29 mg, 0.217 mmol) and N,N-diisopropylethylamine (88 μL, 0.506 mmol) in dichloromethane (2.9 mL). The crude was purified by flash column chromatography (SiO₂, 3-4% methanol in dichloromethane) to give 169 as a white solid (25 mg, 55%). [α]_{D}²⁴=−28.68° (c=1, MeOH); ¹H NMR (600 MHz, CDCl₃) δ 7.22-7.18 (m, 2H), 7.17-7.13 (m, 2H), 6.97 (d, 1H), 6.94 (d, 1H), 6.92 (s, 1H), 6.30 (br s, 1H), 3.84 (d, 1H), 3.71 (d, 1H), 3.52 (dt, 1H), 3.40 (ddd, 1H), 3.28-3.11 (m, 5H), 2.95-2.88 (m, 1H), 2.78 (dd, 1H), 2.72 (dd, 1H), 2.40 (s, 3H), 2.30 (s, 3H); LC-MS (ESI) m/z 335.2 [M+H]⁺.

### Example 170: N-(((3S)-2,6-Dimethyl-1,2,3,4-tetrahydroisoquinolin-3-yl)methyl)-1,2,3,4-tetrahydronaphthalene-2-carboxamide (170)

The compound was prepared by general procedure i1 with bix (36.8 mg, 0.193 mmol), 1,2,3,4-tetrahydronaphthalene-2-carboxylic acid (32.3 mg, 0.183 mmol), 1-(3-dimethylaminopropyl)-3-ethylcarbodiimide hydrochloride (59 mg, 0.29 mmol), 1-hydroxybenzotriazole hydrate (39 mg, 0.29 mmol) and N,N-diisopropylethylamine (120 μL, 0.676 mmol) in dichloromethane (3.9 mL). The crude was purified by flash column chromatography (SiO₂, 3% methanol in dichloromethane) to give 170 as a yellow syrup (43 mg, 68%). ¹H NMR (600 MHz, CDCl₃) δ 7.15-7.02 (m, 4H), 6.97 (d, 1H), 6.95-6.90 (m, 2H), 6.34 (br s, 1H), 3.82 (dd, 1H), 3.68 (d, 1H), 3.54-3.47 (m, 1H), 3.44-3.36 (m, 1H), 3.07-2.99 (m, 1H), 2.95-2.80 (m, 4H), 2.80-2.68 (m, 2H), 2.59-2.49 (m overlapped with H₂O, 1H), 2.39 (s, 1.5H), 2.39 (s, 1.5H), 2.30 (s, 3H), 2.12-2.06 (m, 1H), 1.94-1.86 (m, 1H); LC-MS (ESI) m/z 349.3 [M+H]⁺.

### Example 171: N-(((3S)-2,6-Dimethyl-1,2,3,4-tetrahydroisoquinolin-3-yl)methyl)-5-fluoro-2,3-dihydro-1H-indene-2-carboxamide (171)

The compound was prepared by general procedure i1 with bix (45 mg, 0.236 mmol), 5-fluoro-2,3-dihydro-1H-indene-2-carboxylic acid (40 mg, 0.225 mmol), 1-(3-dimethylaminopropyl)-3-ethylcarbodiimide hydrochloride (68 mg, 0.355 mmol), 1-hydroxybenzotriazole hydrate (48 mg, 0.355 mmol) and N,N-diisopropylethylamine (144 μL, 0.828 mmol) in dichloromethane (4.7 mL). The crude was purified by flash column chromatography (SiO₂, 3% methanol in dichloromethane) to give 171 as a white solid (45 mg, 57%). ¹H NMR (600 MHz, DMSO-d₆) δ 7.94 (br t, 1H), 7.18 (t, 1H), 7.01 (d, 1H), 6.95-6.90 (m, 3H), 6.89 (s, 1H), 3.72 (d, 1H), 3.51 (d, 1H), 3.38 (dt, 1H), 3.26 (quin., 1H), 3.10-2.99 (m, 4H), 2.97 (dd, 1H), 2.76-2.67 (m, 2H), 2.64-2.57 (m, 1H), 2.36 (s, 3H), 2.23 (s, 3H); LC-MS (ESI) m/z 353.2 [M+H]⁺.

### Example 172: (3S)—N-(((3S)-2,6-Dimethyl-1,2,3,4-tetrahydroisoquinolin-3-yl)methyl)-2-methyl-1,2,3,4-tetrahydroisoquinoline-3-carboxamide (172)

The compound was prepared by general procedure i1 with bix (45 mg, 0.236 mmol), (3S)-2-methyl-1,2,3,4-tetrahydroisoquinoline-3-carboxylic acid (43 mg, 0.225 mmol), 1-(3-dimethylamino-propyl)-3-ethylcarbodiimide hydrochloride (68 mg, 0.355 mmol), 1-hydroxybenzotriazole hydrate (48 mg, 0.355 mmol) and N,N-diisopropylethylamine (144 μL, 0.828 mmol) in dichloromethane (4.7 mL). The crude was purified by flash column chromatography (SiO₂, 2-3% methanol in dichloromethane) to give 172 as a beige solid (45 mg, 55%). ¹H NMR (600 MHz, DMSO-d₆) δ 7.90 (t, 1H), 7.15-7.09 (m, 3H), 7.08-7.04 (m, 1H), 6.95-6.90 (m, 2H), 6.86 (s, 1H), 3.87 (d, 1H), 3.71 (d, 1H), 3.55-3.46 (m, 2H), 3.31-3.25 (m overlapped with H₂O, 1H), 3.18-3.09 (m, 2H), 2.94 (dd, 1H), 2.85 (dd, 1H), 2.75-2.64 (m, 2H), 2.56 (dd, 1H), 2.31 (s, 6H), 2.23 (s, 3H); LC-MS (ESI) m/z 364.2 [M+H]⁺.

### Example 173: N-(((3S)-2,6-Dimethyl-1,2,3,4-tetrahydroisoquinolin-3-yl)methyl)-1H-indene-2-carboxamide (173)

The compound was prepared by general procedure i1 with bix (36.8 mg, 0.193 mmol), 1H-indene-2-carboxylic acid (29.4 mg, 0.183 mmol), 1-(3-dimethylaminopropyl)-3-ethylcarbodiimide hydrochloride (59 mg, 0.29 mmol), 1-hydroxybenzotriazole hydrate (39 mg, 0.29 mmol) and N,N-diisopropylethylamine (120 μL, 0.676 mmol) in dichloromethane (3.9 mL). The crude was purified by flash column chromatography (SiO₂, 3% methanol in dichloromethane) to give 173 as a yellow syrup (30 mg, 49%). ¹H NMR (600 MHz, CDCl₃) δ 7.49-7.44 (m, 3H), 7.33-7.27 (m, 2H), 6.99-6.94 (m, 2H), 6.92 (s, 1H), 6.74 (br s, 1H), 3.88 (d, 1H), 3.74 (d, 1H), 3.71-3.59 (m, 3H), 3.51 (ddd, 1H), 3.04-2.98 (m, 1H), 2.86-2.75 (m, 2H), 2.46 (s, 3H), 2.27 (s, 3H); LC-MS (ESI) m/z 333.3 [M+H]⁺.

### Example 174: (2R)—N-(((3S)-2,6-Dimethyl-1,2,3,4-tetrahydroisoquinolin-3-yl)methyl)-1,2,3,4-tetrahydronaphthalene-2-carboxamide (174)

The compound was prepared by general procedure i1 with bix (37.5 mg, 0.197 mmol), (2R)-1,2,3,4-tetrahydronaphthalene-2-carboxylic acid (33 mg, 0.187 mmol), 1-(3-dimethylaminopropyl)-3-ethylcarbodiimide hydrochloride (57 mg, 0.296 mmol), 1-hydroxybenzotriazole hydrate (40 mg, 0.296 mmol) and N,N-diisopropylethylamine (120 μL, 0.69 mmol) in dichloromethane (4 mL). The crude was purified by flash column chromatography (SiO₂, 3% methanol in dichloromethane) to give 174 as a yellow syrup (47 mg, 72%). ¹H NMR (600 MHz, DMSO-d₆) δ 7.88 (br t, 1H), 7.10-7.03 (m, 4H), 6.94-6.91 (m, 2H), 6.90 (s, 1H), 3.73 (d, 1H), 3.52 (d, 1H), 3.41 (dt, 1H), 3.09-3.01 (m, 1H), 2.84 (dd, 1H), 2.81-2.68 (m, 5H), 2.65-2.59 (m, 1H), 2.55 (tdd, 1H), 2.37 (s, 3H), 2.24 (s, 3H), 1.96-1.90 (m, 1H), 1.67 (dtd, 1H); LC-MS (ESI) m/z 349.2 [M+H]⁺.

### Example 175: (2S)—N-(((3S)-2,6-Dimethyl-1,2,3,4-tetrahydroisoquinolin-3-yl)methyl)-1,2,3,4-tetrahydronaphthalene-2-carboxamide (175)

The compound was prepared by general procedure i1 with bix (37.5 mg, 0.197 mmol), (2S)-1,2,3,4-tetrahydronaphthalene-2-carboxylic acid (33 mg, 0.187 mmol), 1-(3-dimethylaminopropyl)-3-ethylcarbodiimide hydrochloride (57 mg, 0.296 mmol), 1-hydroxybenzotriazole hydrate (40 mg, 0.296 mmol) and N,N-diisopropylethylamine (120 μL, 0.69 mmol) in dichloromethane (4 mL). The crude was purified by flash column chromatography (SiO₂, 3% methanol in dichloromethane) to give 175 as a yellow syrup (45 mg, 69%). [α]_{D}²⁴=−2.66° (c=1, MeOH); ¹H NMR (600 MHz, DMSO-d₆) δ 7.87 (br t, 1H), 7.11-7.01 (m, 4H), 6.94-6.91 (m, 2H), 6.89 (s, 1H), 3.72 (d, 1H), 3.51 (d, 1H), 3.40-3.36 (m, 1H), 3.13-3.04 (m, 1H), 2.86-2.68 (m, 6H), 2.65-2.59 (m, 1H), 2.55 (tdd, 1H), 2.36 (s, 3H), 2.24 (s, 3H), 1.96-1.90 (m, 1H), 1.68 (dtd, 1H); LC-MS (ESI) m/z 349.2 [M+H]⁺.

### Example 176: 3-(3-Fluorophenyl)-N-((6-methoxy-2-methyl-1,2,3,4-tetrahydroisoquinolin-3-yl)methyl)propenamide (176)

The compound was prepared by general procedure i1 with cix (72.7 mg, 0.352 mmol), 3-(3-fluorophenyl) propanoic acid (56.3 mg, 0.33 mmol), EDCI (87.6 mg, 0.458 mmol), HOBt (52.4 mg, 0.388 mmol) and DIPEA (87 μL, 0.529 mmol) in CH₂Cl₂ (4 mL), stirred at room temperature for 17 h. After workup, the crude was purified by Isco Combi-Flash Companion column chromatography (0-5% methanol in CH₂Cl₂) to give 176 (94.8 mg, 76%). ¹H NMR (400 MHz, DMSO-d₆) δ 7.80 (br t, 1H), 7.35-7.26 (m, 1H), 7.09-6.91 (m, 4H), 6.69 (dd, 1H), 6.60 (d, 1H), 3.71 (s, 3H), 3.66 (d, 1H), 3.44 (d, 1H), 3.33-3.26 (m overlapped with H₂O, 1H), 3.03 (ddd, 1H), 2.84 (t, 2H), 2.67-2.52 (m, 3H), 2.43 (t, 2H), 2.29 (s, 3H); LC-MS (ESI) m/z 357.2 [M+H]⁺.

### Example 177: N-((6-Methoxy-2-methyl-1,2,3,4-tetrahydroisoquinolin-3-yl)methyl)-3-phenylpropanamide (177)

The compound was prepared by general procedure i1 with cix (72.7 mg, 0.352 mmol), 3-phenylpropanoic acid (50.3 mg, 0.33 mmol), EDCI (87.6 mg, 0.458 mmol), HOBt (52.4 mg, 0.388 mmol) and DIPEA (87 μL, 0.529 mmol) in CH₂Cl₂ (4 mL), stirred at room temperature for 17 h. After workup, the crude was purified by Isco Combi-Flash Companion column chromatography (0-5% methanol in CH₂Cl₂) to give 177 (87.2 mg, 73%). ¹H NMR (400 MHz, DMSO-d₆) δ 7.79 (br t, 1H), 7.32-7.14 (m, 5H), 6.95 (d, 1H), 6.69 (dd, 1H), 6.60 (d, 1H), 3.71 (s, 3H), 3.66 (d, 1H), 3.44 (d, 1H), 3.37-3.27 (m overlapped with H₂O, 1H), 3.02 (ddd, 1H), 2.82 (t, 2H), 2.70-2.53 (m, 3H), 2.41 (t, 2H), 2.30 (s, 3H); LC-MS (ESI) m/z 339.2 [M+H]⁺.

### Example 178: 3-Cyclohexyl-N-((6-methoxy-2-methyl-1,2,3,4-tetrahydroisoquinolin-3-yl)methyl)propenamide (178)

The compound was prepared by general procedure i1 with cix (72.7 mg, 0.352 mmol), 3-cyclohexylpropanoic acid (48 μL, 0.33 mmol), EDCI (87.6 mg, 0.458 mmol), HOBt (52.4 mg, 0.388 mmol) and DIPEA (87 μL, 0.529 mmol) in CH₂Cl₂ (4 mL), stirred at room temperature for 17 h. After workup, the crude was purified by Isco Combi-Flash Companion column chromatography (0-5% methanol in CH₂Cl₂) to give 178 (83.8 mg, 69%). ¹H NMR (400 MHz, DMSO-d₆) δ 7.75 (br t, 1H), 6.96 (d, 1H), 6.70 (dd, 1H), 6.63 (d, 1H), 3.71 (s, 3H), 3.68 (d, 1H), 3.46 (d, 1H), 3.36-3.28 (m overlapped with H₂O, 1H), 3.03 (ddd, 1H), 2.78-2.55 (m, 3H), 2.33 (s, 3H), 2.10 (t, 2H), 1.78-1.54 (m, 5H), 1.39 (q, 2H), 1.30-1.04 (m, 4H), 0.94-0.77 (m, 2H); LC-MS (ESI) m/z 345.3 [M+H]⁺.

### Example 179: 3-(3-Fluorophenyl)-N-(((3S)-6-methoxy-2-methyl-1,2,3,4-tetrahydroisoquinolin-3-yl)methyl)propenamide (179)

The compound was prepared by general procedure i1 with dix (73 mg, 0.354 mmol), 3-(3-fluorophenyl) propanoic acid (56.5 mg, 0.33 mmol), EDCI (87.9 mg, 0.460 mmol), HOBt (52.6 mg, 0.389 mmol) and DIPEA (88 μL, 0.53 mmol) in CH₂Cl₂ (4 mL), stirred at room temperature for 17 h. After workup, the crude was purified by Isco Combi-Flash Companion column chromatography (0-5% methanol in CH₂Cl₂) to give 179 (91.1 mg, 72%). ¹H NMR (400 MHz, DMSO-d₆) δ 7.80 (br t, 1H), 7.35-7.26 (m, 1H), 7.09-7.03 (m, 2H), 7.03-6.96 (m, 1H), 6.95 (d, 1H), 6.69 (dd, 1H), 6.59 (d, 1H), 3.71 (s, 3H), 3.66 (d, 1H), 3.44 (d, 1H), 3.32-3.26 (m, 1H), 3.03 (ddd, 1H), 2.84 (t, 2H), 2.68-2.53 (m, 3H), 2.45 (t, 2H), 2.29 (s, 3H); LC-MS (ESI) m/z 357.2 [M+H]⁺.

### Example 180: N-(((3S)-6-Methoxy-2-methyl-1,2,3,4-tetrahydroisoquinolin-3-yl)methyl)-3-phenylpropanamide (180)

The compound was prepared by general procedure i1 with dix (73 mg, 0.354 mmol), 3-phenylpropanoic acid (50.5 mg, 0.33 mmol), EDCI (87.9 mg, 0.460 mmol), HOBt (52.6 mg, 0.389 mmol) and DIPEA (88 μL, 0.53 mmol) in CH₂Cl₂ (4 mL), stirred at room temperature for 17 h. After workup, the crude was purified by Isco Combi-Flash Companion column chromatography (0-5% methanol in CH₂Cl₂) to give 180 (87.6 mg, 73%). ¹H NMR (400 MHz, DMSO-d₆) δ 7.79 (br t, 1H), 7.32-7.12 (m, 5H), 6.95 (d, 1H), 6.69 (dd, 1H), 6.60 (d, 1H), 3.71 (s, 3H), 3.66 (d, 1H), 3.44 (d, 1H), 3.36-3.27 (m overlapped with H₂O, 1H), 3.02 (ddd, 1H), 2.82 (t, 2H), 2.71-2.53 (m, 3H), 2.41 (t, 2H), 2.30 (s, 3H); LC-MS (ESI) m/z 339.2 [M+H]⁺.

### Example 181: 3-Cyclohexyl-N-(((3S)-6-methoxy-2-methyl-1,2,3,4-tetrahydroisoquinolin-3-yl)methyl)propenamide (181)

The compound was prepared by general procedure i1 with dix (73 mg, 0.354 mmol), 3-cyclohexylpropanoic acid (48.5 μL, 0.283 mmol), EDCI (87.9 mg, 0.460 mmol), HOBt (52.6 mg, 0.389 mmol) and DIPEA (88 μL, 0.53 mmol) in CH₂Cl₂ (4 mL), stirred at room temperature for 17 h. After workup, the crude was purified by Isco Combi-Flash Companion column chromatography (0-5% methanol in CH₂Cl₂) to give 181 (89.4 mg, 73%). ¹H NMR (400 MHz, DMSO-d₆) δ 7.75 (br t, 1H), 6.96 (d, 1H), 6.70 (dd, 1H), 6.63 (d, 1H), 3.71 (s, 3H), 3.68 (d, 1H), 3.46 (d, 1H), 3.37-3.28 (m overlapped with H₂O, 1H), 3.02 (ddd, 1H), 2.78-2.55 (m, 3H), 2.33 (s, 3H), 2.10 (t, 2H), 1.72-1.57 (m, 5H), 1.39 (q, 2H), 1.26-1.05 (m, 4H), 0.93-0.78 (m, 2H); LC-MS (ESI) m/z 345.3 [M+H]⁺.

### Example 182: N-(((3R)-2,7-Dimethyl-1,2,3,4-tetrahydroisoquinolin-3-yl)methyl)-4-(trifluoromethyl)benzamide (182)

The compound was prepared by general procedure i1 with iix (48.5 mg, 0.255 mmol), 4-(trifluoromethyl)benzoic acid (46 mg, 0.242 mmol), EDCI (63.3 mg, 0.331 mmol), HOBt (37.9 mg, 0.28 mmol) and DIPEA (63.2 μL, 0.382 mmol) in CH₂Cl₂ (4 mL), stirred at room temperature for 17 h. After workup, the crude was purified by Isco Combi-Flash Companion column chromatography (0-5% methanol in CH₂Cl₂) to give 182 (70.2 mg, 76%). ¹H NMR (400 MHz, DMSO-d₆) δ 8.68 (br t, 1H), 8.04 (d, 2H), 7.86 (d, 2H), 6.98 (d, 1H), 6.94 (d, 1H), 6.87 (s, 1H), 3.74 (d, 1H), 3.63-3.51 (m, 2H), 3.22 (ddd, 1H), 2.93 (ddd, 1H), 2.79 (dd, 1H), 2.64 (dd, 1H), 2.41 (s, 3H), 2.24 (s, 3H); LC-MS (ESI) m/z 363.1 [M+H]⁺.

### Example 183: N-(((3R)-2,7-Dimethyl-1,2,3,4-tetrahydroisoquinolin-3-yl)methyl)-4-(ethylsulfanyl)benzamide (183)

The compound was prepared by general procedure i1 with iix (48.5 mg, 0.255 mmol), 4-(ethylsulfanyl)benzoic acid (44.1 mg, 0.242 mmol), EDCI (63.3 mg, 0.331 mmol), HOBt (37.9 mg, 0.28 mmol) and DIPEA (63.2 μL, 0.382 mmol) in CH₂Cl₂ (4 mL), stirred at room temperature for 17 h. After workup, the crude was purified by Isco Combi-Flash Companion column chromatography (0-5% methanol in CH₂Cl₂) to give 183 (63.3 mg, 70%). ¹H NMR (400 MHz, DMSO-d₆) δ 8.38 (br t, 1H), 7.78 (d, 2H), 7.36 (d, 2H), 6.98 (d, 1H), 6.93 (d, 1H), 6.86 (s, 1H), 3.73 (d, 1H), 3.62-3.48 (m, 2H), 3.18 (ddd, 1H), 3.05 (q, 2H), 2.90 (ddd, 1H), 2.77 (dd, 1H), 2.62 (dd, 1H), 2.40 (s, 3H), 2.24 (s, 3H), 1.27 (t, 3H); LC-MS (ESI) m/z 355.2 [M+H]⁺.

### Example 184: 3,4-Dichloro-N-(((3R)-2,7-dimethyl-1,2,3,4-tetrahydroisoquinolin-3-yl)methyl)benzamide (184)

The compound was prepared by general procedure i1 with iix (48.5 mg, 0.255 mmol), 3,4-dichlorobenzoic acid (46 mg, 0.242 mmol), EDCI (63.3 mg, 0.331 mmol), HOBt (37.9 mg, 0.28 mmol) and DIPEA (63.2 μL, 0.382 mmol) in CH₂Cl₂ (4 mL), stirred at room temperature for 17 h. After workup, the crude was purified by Isco Combi-Flash Companion column chromatography (0-5% methanol in CH₂Cl₂) to give 184 (68.5 mg, 74%). ¹H NMR (400 MHz, DMSO-d₆) δ 8.63 (br t, 1H), 8.06 (d, 1H), 7.83 (dd, 1H), 7.76 (d, 1H), 6.98 (d, 1H), 6.93 (d, 1H), 6.86 (s, 1H), 3.73 (d, 1H), 3.60-3.48 (m, 2H), 3.20 (ddd, 1H), 2.91 (ddd, 1H), 2.78 (dd, 1H), 2.62 (dd, 1H), 2.40 (s, 3H), 2.24 (s, 3H); LC-MS (ESI) m/z 364.1 [M+H]⁺.

### Example 185: N-(((3S)-2,7-Dimethyl-1, 2, 3, 4-tetrahydroisoquinolin-3-yl)methyl)-4-(trifluoromethyl)benzamide (185)

The compound was prepared by general procedure i1 with jix (53.3 mg, 0.28 mmol), 4-(trifluoromethyl)benzoic acid (50.6 mg, 0.266 mmol), EDCI (69.6 mg, 0.364 mmol), HOBt (41.6 mg, 0.308 mmol) and DIPEA (70 μL, 0.42 mmol) in CH₂Cl₂ (4 mL), stirred at room temperature for 17 h. After workup, the crude was purified by Isco Combi-Flash Companion column chromatography (0-5% methanol in CH₂Cl₂) to give 185 (80.3 mg, 79%). ¹H NMR (400 MHz, DMSO-d₆) δ 8.68 (br t, 1H), 8.04 (d, 2H), 7.86 (d, 2H), 6.98 (d, 1H), 6.94 (d, 1H), 6.87 (s, 1H), 3.74 (d, 1H), 3.64-3.51 (m, 2H), 3.22 (ddd, 1H), 2.93 (ddd, 1H), 2.79 (dd, 1H), 2.64 (dd, 1H), 2.41 (s, 3H), 2.24 (s, 3H); LC-MS (ESI) m/z 363.1 [M+H]⁺.

### Example 186: N-(((3S)-2,7-Dimethyl-1,2,3,4-tetrahydroisoquinolin-3-yl)methyl)-4-(ethylsulfanyl)benzamide (186)

The compound was prepared by general procedure i1 with jix (53.3 mg, 0.28 mmol), 4-(ethylsulfanyl)benzoic acid (48.5 mg, 0.266 mmol), EDCI (69.6 mg, 0.364 mmol), HOBt (41.6 mg, 0.308 mmol) and DIPEA (70 μL, 0.42 mmol) in CH₂Cl₂ (4 mL), stirred at room temperature for 17 h. After workup, the crude was purified by Isco Combi-Flash Companion column chromatography (0-5% methanol in CH₂Cl₂) to give 186 (76.4 mg, 77%). ¹H NMR (400 MHz, DMSO-d₆) δ 8.38 (br t, 1H), 7.78 (d, 2H), 7.36 (d, 2H), 6.98 (d, 1H), 6.93 (d, 1H), 6.87 (s, 1H), 3.74 (d, 1H), 3.62-3.47 (m, 2H), 3.18 (ddd, 1H), 3.05 (q, 2H), 2.97-2.85 (m, 1H), 2.77 (dd, 1H), 2.62 (dd, 1H), 2.40 (s, 3H), 2.24 (s, 3H), 1.27 (t, 3H); LC-MS (ESI) m/z 355.2 [M+H]⁺.

### Example 187: 3,4-Dichloro-N-(((3S)-2,7-dimethyl-1,2,3,4-tetrahydroisoquinolin-3-yl)methyl)benzamide (187)

The compound was prepared by general procedure i1 with jix (53.3 mg, 0.28 mmol), 3,4-dichlorobenzoic acid (50.8 mg, 0.266 mmol), EDCI (69.6 mg, 0.364 mmol), HOBt (41.6 mg, 0.308 mmol) and DIPEA (70 μL, 0.42 mmol) in CH₂Cl₂ (4 mL), stirred at room temperature for 17 h. After workup, the crude was purified by Isco Combi-Flash Companion column chromatography (0-5% methanol in CH₂Cl₂) to give 187 (80.5 mg, 79%). ¹H NMR (400 MHz, DMSO-d₆) δ 8.63 (br t, 1H), 8.06 (d, 1H), 7.83 (dd, 1H), 7.76 (d, 1H), 6.98 (d, 1H), 6.93 (d, 1H), 6.86 (s, 1H), 3.73 (d, 1H), 3.60-3.50 (m, 2H), 3.20 (ddd, 1H), 2.91 (ddd, 1H), 2.78 (dd, 1H), 2.62 (dd, 1H), 2.40 (s, 3H), 2.24 (s, 3H); LC-MS (ESI) m/z 364.1 [M+H]⁺.

### Example 188: N-((5-Chloro-2-methyl-1,2,3,4-tetrahydroisoquinolin-3-yl)methyl)-1H-indene-2-carboxamide (188)

The compound was prepared by general procedure i1 with Fix (41 mg, 0.195 mmol), 1H-indene-2-carboxylic acid (29.6 mg, 0.185 mmol), EDCI (48.3 mg, 0.253 mmol), HOBt (28.9 mg, 0.214 mmol) and DIPEA (48 μL, 0.292 mmol) in CH₂Cl₂ (4 mL), stirred at room temperature for 17 h. After workup, the crude was purified by Isco Combi-Flash Companion column chromatography (0-5% methanol in CH₂Cl₂) to give 188 (79.4 mg, 79%). ¹H NMR (400 MHz, DMSO-d₆) δ 8.18 (br t, 1H), 7.60-7.49 (m, 3H), 7.37-7.25 (m, 3H), 7.18 (t, 1H), 7.08 (d, 1H), 3.83 (d, 1H), 3.66 (d, 2H), 3.62 (d, 1H), 3.54 (dt, 1H), 3.24 (ddd, 1H), 2.95 (ddd, 1H), 2.79 (dd, 1H), 2.64 (dd, 1H), 2.40 (s, 3H); LC-MS (ESI) m/z 353.2 [M+H]⁺.

### Example 189: N-(((3R)-5-Chloro-2-methyl-1,2,3,4-tetrahydroisoquinolin-3-yl)methyl)-1H-indene-2-carboxamide (189)

The compound was prepared by general procedure i1 with Gix (44 mg, 0.209 mmol), 1H-indene-2-carboxylic acid (31.8 mg, 0.198 mmol), EDCI (51.9 mg, 0.271 mmol), HOBt (31 mg, 0.23 mmol) and DIPEA (52 μL, 0.313 mmol) in CH₂Cl₂ (4 mL), stirred at room temperature for 17 h. After workup, the crude was purified by Isco Combi-Flash Companion column chromatography (0-5% methanol in CH₂Cl₂) to give 189 (58 mg, 79%). ¹H NMR (400 MHz, DMSO-d₆) δ 8.18 (br t, 1H), 7.61-7.49 (m, 3H), 7.38-7.25 (m, 3H), 7.18 (t, 1H), 7.08 (d, 1H), 3.83 (d, 1H), 3.66 (d, 2H), 3.62 (d, 1H), 3.54 (dt, 1H), 3.24 (ddd, 1H), 2.95 (ddd, 1H), 2.79 (dd, 1H), 2.64 (dd, 1H), 2.40 (s, 3H); LC-MS (ESI) m/z 353.1 [M+H]⁺.

### Example 190: (2R)—N-(((3R)-5-chloro-2-methyl-1,2,3,4-tetrahydroisoquinolin-3-yl)methyl)-1,2,3,4-tetrahydronaphthalene-2-carboxamide (190)

The compound was prepared by general procedure i1 with Gix (44 mg, 0.209 mmol), (2R)-1,2,3,4-tetrahydronaphthalene-2-carboxylic acid (35 mg, 0.198 mmol), EDCI (51.9 mg, 0.271 mmol), HOBt (31 mg, 0.23 mmol) and DIPEA (52 μL, 0.313 mmol) in CH₂Cl₂ (4 mL), stirred at room temperature for 17 h. After workup, the crude was purified by Isco Combi-Flash Companion column chromatography (0-5% methanol in CH₂Cl₂) to give 190 (55 mg, 71%). ¹H NMR (400 MHz, DMSO-d₆) δ 7.94 (br t, 1H), 7.29 (d, 1H), 7.18 (t, 1H), 7.12-7.03 (m, 5H), 3.81 (d, 1H), 3.57 (d, 1H), 3.38 (dt, 1H), 3.21 (ddd, 1H), 2.92-2.71 (m, 6H), 2.65-2.53 (m, 2H), 2.36 (s, 3H), 2.00-1.90 (m, 1H), 1.77-1.63 (m, 1H); LC-MS (ESI) m/z 369.1 [M+H]⁺.

### Example 191: (2S)—N-(((3R)-5-Chloro-2-methyl-1,2,3,4-tetrahydroisoquinolin-3-yl)methyl)-1,2,3,4-tetrahydronaphthalene-2-carboxamide (191)

The compound was prepared by general procedure i1 with Gix (44 mg, 0.209 mmol), (25)-1,2,3,4-tetrahydronaphthalene-2-carboxylic acid (35 mg, 0.198 mmol), EDCI (51.9 mg, 0.271 mmol), HOBt (31 mg, 0.23 mmol) and DIPEA (52 μL, 0.313 mmol) in CH₂Cl₂ (4 mL), stirred at room temperature for 17 h. After workup, the crude was purified by Isco Combi-Flash Companion column chromatography (0-5% methanol in CH₂Cl₂) to give 191 (57.7 mg, 75%). ¹H NMR (400 MHz, DMSO-d₆) δ 7.94 (br t, 1H), 7.28 (d, 1H), 7.17 (t, 1H), 7.12-7.03 (m, 5H), 3.81 (d, 1H), 3.57 (d, 1H), 3.43 (ddd, 1H), 3.17 (ddd, 1H), 2.93-2.69 (m, 6H), 2.66-2.54 (m, 2H), 2.36 (s, 3H), 2.01-1.88 (m, 1H), 1.78-1.62 (m, 1H); LC-MS (ESI) m/z 369.1 [M+H]⁺.

### Example 192: N-(((3S)-5-Chloro-2-methyl-1,2,3,4-tetrahydroisoquinolin-3-yl)methyl)-1H-indene-2-carboxamide (192)

The compound was prepared by general procedure i1 with aix (44 mg, 0.209 mmol), 1H-indene-2-carboxylic acid (31.8 mg, 0.198 mmol), EDCI (51.9 mg, 0.271 mmol), HOBt (31 mg, 0.23 mmol) and DIPEA (52 μL, 0.313 mmol) in CH₂Cl₂ (4 mL), stirred at room temperature for 17 h. After workup, the crude was purified by Isco Combi-Flash Companion column chromatography (0-5% methanol in CH₂Cl₂) to give 192 (62.8 mg, 85%). ¹H NMR (400 MHz, DMSO-d₆) δ 8.18 (br t, 1H), 7.60-7.49 (m, 3H), 7.36-7.24 (m, 3H), 7.18 (t, 1H), 7.08 (d, 1H), 3.83 (d, 1H), 3.66 (d, 2H), 3.62 (d, 1H), 3.54 (dt, 1H), 3.24 (ddd, 1H), 2.95 (ddd, 1H), 2.79 (dd, 1H), 2.64 (dd, 1H), 2.40 (s, 3H); LC-MS (ESI) m/z 353.1 [M+H]⁺.

### Example 193: (2R)—N-(((3S)-5-Chloro-2-methyl-1,2,3,4-tetrahydroisoquinolin-3-yl)methyl)-1,2,3,4-tetrahydronaphthalene-2-carboxamide (193)

The compound was prepared by general procedure i1 with aix (44 mg, 0.209 mmol), (2R)-1,2,3,4-tetrahydronaphthalene-2-carboxylic acid (35 mg, 0.198 mmol), EDCI (51.9 mg, 0.271 mmol), HOBt (31 mg, 0.23 mmol) and DIPEA (52 μL, 0.313 mmol) in CH₂Cl₂ (4 mL), stirred at room temperature for 17 h. After workup, the crude was purified by Isco Combi-Flash Companion column chromatography (0-5% methanol in CH₂Cl₂) to 193 (63.1 mg, 82%). ¹H NMR (400 MHz, DMSO-d₆) δ 7.94 (br t, 1H), 7.29 (d, 1H), 7.18 (t, 1H), 7.12-7.01 (m, 5H), 3.81 (d, 1H), 3.57 (d, 1H), 3.43 (dt, 1H), 3.17 (ddd, 1H), 2.93-2.68 (m, 6H), 2.65-2.53 (m, 2H), 2.36 (s, 3H), 2.00-1.87 (m, 1H), 1.78-1.62 (m, 1H); LC-MS (ESI) m/z 369.1 [M+H]⁺.

### Example 194: (2S)—N-(((3S)-5-Chloro-2-methyl-1,2,3,4-tetrahydroisoquinolin-3-yl)methyl)-1,2,3,4-tetrahydronaphthalene-2-carboxamide (194)

The compound was prepared by general procedure i1 with aix (44 mg, 0.209 mmol), (25)-1,2,3,4-tetrahydronaphthalene-2-carboxylic acid (35 mg, 0.198 mmol), EDCI (51.9 mg, 0.271 mmol), HOBt (31 mg, 0.23 mmol) and DIPEA (52 μL, 0.313 mmol) in CH₂Cl₂ (4 mL), stirred at room temperature for 17 h. After workup, the crude was purified by Isco Combi-Flash Companion column chromatography (0-5% methanol in CH₂Cl₂) to give 194 (62.8 mg, 82%). ¹H NMR (400 MHz, DMSO-d₆) δ 7.94 (br t, 1H), 7.28 (d, 1H), 7.17 (t, 1H), 7.09-7.01 (m, 5H), 3.81 (d, 1H), 3.57 (d, 1H), 3.38 (dt, 1H), 3.21 (ddd, 1H), 2.93-2.68 (m, 6H), 2.67-2.54 (m, 2H), 2.36 (s, 3H), 2.03-1.89 (m, 1H), 1.80-1.62 (m, 1H); LC-MS (ESI) m/z 369.2 [M+H]⁺.

### Example 195: N-(((3S)-5-Chloro-2-methyl-1,2,3,4-tetrahydroisoquinolin-3-yl)methyl)-1,2,3,4-tetrahydronaphthalene-2-carboxamide (195)

The compound was prepared by general procedure i1 with aix (44 mg, 0.209 mmol), 1,2,3,4-tetrahydronaphthalene-2-carboxylic acid (35 mg, 0.198 mmol), EDCI (51.9 mg, 0.271 mmol), HOBt (31 mg, 0.23 mmol) and DIPEA (52 μL, 0.313 mmol) in CH₂Cl₂ (4 mL), stirred at room temperature for 17 h. After workup, the crude was purified by Isco Combi-Flash Companion column chromatography (0-5% methanol in CH₂Cl₂) to give 195 (60.6 mg, 79%). ¹H NMR (400 MHz, DMSO-d₆) δ 7.94 (br t, 1H), 7.28 (d, 1H), 7.18 (t, 1H), 7.12-7.03 (m, 5H), 3.81 (d, 1H), 3.57 (d, 1H), 3.49-3.35 (m, 1H), 3.27-3.11 (m, 1H), 2.97-2.70 (m, 6H), 2.68-2.54 (m, 2H), 2.36 (s, 3H), 2.03-1.88 (m, 1H), 1.80-1.59 (m, 1H); LC-MS (ESI) m/z 369.1 [M+H]⁺.

### Example 196: N-(((3S)-5-Chloro-2-methyl-1,2,3,4-tetrahydroisoquinolin-3-yl)methyl)-2,3-dihydro-1H-indene-2-carboxamide (196)

The compound was prepared by general procedure i1 with aix (44 mg, 0.209 mmol), 2,3-dihydro-1H-indene-2-carboxylic acid (32.2 mg, 0.198 mmol), EDCI (51.9 mg, 0.271 mmol), HOBt (31 mg, 0.23 mmol) and DIPEA (52 μL, 0.313 mmol) in CH₂Cl₂ (4 mL), stirred at room temperature for 17 h. After workup, the crude was purified by Isco Combi-Flash Companion column chromatography (0-5% methanol in CH₂Cl₂) to give 196 (57.8 mg, 78%). ¹H NMR (400 MHz, DMSO-d₆) δ 8.00 (br t, 1H), 7.28 (d, 1H), 7.23-7.05 (m, 6H), 3.81 (d, 1H), 3.58 (d, 1H), 3.41 (dt, 1H), 3.29-3.14 (m, 2H), 3.07 (d, 2H), 3.05 (d, 2H), 2.83-2.70 (m, 2H), 2.59 (dd, 1H), 2.35 (s, 3H); LC-MS (ESI) m/z 355.1 [M+H]⁺.

### Example 197: N-((6-Fluoro-2-methyl-1,2,3,4-tetrahydroisoquinolin-3-yl)methyl)-2,3-dihydro-1H-indene-2-carboxamide (197)

The compound was prepared by general procedure i1 with Oix (62 mg, 0.319 mmol), 2-indanecarboxylic acid (49 mg, 0.303 mmol), 1-(3-dimethylaminopropyl)-3-ethylcarbodiimide hydrochloride (92 mg, 0.479 mmol), 1-hydroxybenzotriazole hydrate (65 mg, 0.479 mmol) and N,N-diisopropylethylamine (200 μL, 1.12 mmol) in dichloromethane (6.4 mL). The crude was purified by flash column chromatography (SiO₂, 3% methanol in dichloromethane) to give 197 as a beige solid (50 mg, 49%). ¹H NMR (600 MHz, DMSO-d₆) δ 7.94 (br t, 1H), 7.21-7.16 (m, 2H), 7.13-7.10 (m, 2H), 7.09 (dd, 1H), 6.97-6.91 (m, 2H), 3.74 (d, 1H), 3.52 (d, 1H), 3.38 (ddd, 1H), 3.22 (quin., 1H), 3.12-3.06 (m, 1H), 3.06-3.02 (m, 4H), 2.79 (dd, 1H), 2.75-2.69 (m, 1H), 2.66 (dd, 1H), 2.36 (s, 3H); LC-MS (ESI) m/z 339.1 [M+H]⁺.

### Example 198: N-((6-Fluoro-2-methyl-1,2,3,4-tetrahydroisoquinolin-3-yl)methyl)-1,2,3,4-tetrahydronaphthalene-2-carboxamide (198)

The compound was prepared by general procedure i1 with Oix (62 mg, 0.319 mmol), 1,2,3,4-tetrahydronaphthalene-2-carboxylic acid (53 mg, 0.303 mmol), 1-(3-dimethylaminopropyl)-3-ethylcarbodiimide hydrochloride (92 mg, 0.479 mmol), 1-hydroxybenzotriazole hydrate (65 mg, 0.479 mmol) and N,N-diisopropylethylamine (200 μL, 1.12 mmol) in dichloromethane (6.4 mL). The crude was purified by flash column chromatography (SiO₂, 3% methanol in dichloromethane) to give 198 as a yellow syrup (69 mg, 64%). ¹H NMR (600 MHz, DMSO-d₆) δ 7.88 (br t, 1H), 7.13-7.03 (m, 5H), 6.97-6.91 (m, 2H), 3.74 (d, 1H), 3.52 (d, 1H), 3.42-3.34 (m overlapped with H₂O, 1H), 3.13-3.04 (m, 1H), 2.87-2.69 (m, 6H), 2.66 (dd, 1H), 2.59-2.51 (m, 1H), 2.36 (s, 3H), 1.97-1.90 (m, 1H), 1.72-1.63 (m, 1H); LC-MS (ESI) m/z 353.2 [M+H]⁺.

### Example 199: trans-N-((6-Fluoro-2-methyl-1,2,3,4-tetrahydroisoquinolin-3-yl)methyl)-2-phenylcyclopropane-1-carboxamide (199)

The compound was prepared by general procedure i1 with Oix (62 mg, 0.319 mmol), trans-2-phenyl-1-cyclopropanecarboxylic acid (49 mg, 0.303 mmol), 1-(3-dimethylaminopropyl)-3-ethylcarbodiimide hydrochloride (92 mg, 0.479 mmol), 1-hydroxybenzotriazole hydrate (65 mg, 0.479 mmol) and N,N-diisopropylethylamine (200 μL, 1.12 mmol) in dichloromethane (6.4 mL). The crude was purified by flash column chromatography (SiO₂, 3% methanol in dichloromethane) to give 199 as a yellow syrup (68 mg, 66%). ¹H NMR (600 MHz, DMSO-d₆) δ 8.11 (br t, 1H), 7.26 (td, 2H), 7.18-7.15 (m, 1H), 7.13-7.10 (m, 2H), 7.08 (dd, 1H), 6.96-6.90 (m, 2H), 3.73 (d, 1H), 3.51 (d, 1H), 3.41-3.34 (m overlapped with H₂O, 1H), 3.11 (ddt, 1H), 2.76 (d, 1H), 2.72-2.62 (m, 2H), 2.33 (d, 3H), 2.26-2.20 (m, 1H), 1.97 (ddd, 1H), 1.37-1.32 (m, 1H), 1.20-1.15 (m, 1H); LC-MS (ESI) m/z 339.1 [M+H]⁺.

### Example 200: N-((6-Fluoro-2-methyl-1,2,3,4-tetrahydroisoquinolin-3-yl)methyl)-1H-indene-2-carboxamide (200)

The compound was prepared by general procedure i1 with Oix (62 mg, 0.319 mmol), 1H-indene-2-carboxylic acid (48.5 mg, 0.303 mmol), 1-(3-dimethylaminopropyl)-3-ethylcarbodiimide hydrochloride (92 mg, 0.479 mmol), 1-hydroxybenzotriazole hydrate (65 mg, 0.479 mmol) and N,N-diisopropylethylamine (200 μL, 1.12 mmol) in dichloromethane (6.4 mL). The crude was purified by flash column chromatography (SiO₂, 3% methanol in dichloromethane) to give 200 as a brown syrup (50 mg, 49%). ¹H NMR (600 MHz, DMSO-d₆) δ 8.12 (br t, 1H), 7.57-7.50 (m, 3H), 7.34-7.26 (m, 2H), 7.10 (dd, 1H), 6.98-6.91 (m, 2H), 3.76 (d, 1H), 3.65 (d, 2H), 3.56 (d, 1H), 3.50 (dt, 1H), 3.15 (ddd, 1H), 2.89 (quin., 1H), 2.84 (dd, 1H), 2.69 (dd, 1H), 2.41 (s, 3H); LC-MS (ESI) m/z 337.1 [M+H]⁺.

### Example 201: 4-Ethyl-N-(((3R)-6-fluoro-2-methyl-1,2,3,4-tetrahydroisoquinolin-3-yl)methyl)benzamide (201)

The compound was prepared by general procedure i1 with Pix (93 mg, 0.479 mmol), 4-ethylbenzoic acid (68 mg, 0.455 mmol), 1-(3-dimethylaminopropyl)-3-ethylcarbodiimide hydrochloride (138 mg, 0.718 mmol), 1-hydroxybenzotriazole hydrate (97 mg, 0.718 mmol) and N,N-diisopropylethylamine (290 μL, 1.68 mmol) in dichloromethane (9.5 mL). The crude was purified by flash column chromatography (SiO₂, 3% methanol in dichloromethane) to give 201 as a beige solid (100 mg, 67%). ¹H NMR (600 MHz, DMSO-d₆) δ 8.34 (br t, 1H), 7.76 (d, 2H), 7.29 (d, 2H), 7.10 (dd, 1H), 6.97-6.91 (m, 2H), 3.76 (d, 1H), 3.59-3.50 (m, 2H), 3.18 (ddd, 1H), 2.91 (ddd, 1H), 2.83 (dd, 1H), 2.71-2.62 (m, 3H), 2.41 (s, 3H), 1.19 (t, 3H); LC-MS (ESI) m/z 327.1 [M+H]⁺.

### Example 202: N-(((3R)-6-Fluoro-2-methyl-1,2,3,4-tetrahydroisoquinolin-3-yl)methyl)-4-(trifluoromethyl)benzamide (202)

The compound was prepared by general procedure i1 with Pix (93 mg, 0.479 mmol), 4-(trifluoromethyl)benzoic acid (86.5 mg, 0.455 mmol), 1-(3-dimethylaminopropyl)-3-ethylcarbodiimide hydrochloride (138 mg, 0.718 mmol), 1-hydroxybenzotriazole hydrate (97 mg, 0.718 mmol) and N,N-diisopropylethylamine (290 μL, 1.68 mmol) in dichloromethane (9.5 mL). The crude was purified by flash column chromatography (SiO₂, 3% methanol in dichloromethane) to give 202 as a white solid (146 mg, 87%). ¹H NMR (600 MHz, DMSO-d₆) δ 8.68 (br t, 1H), 8.03 (d, 2H), 7.85 (d, 2H), 7.10 (dd, 1H), 6.97-6.92 (m, 2H), 3.76 (d, 1H), 3.60-3.54 (m, 2H), 3.22 (ddd, 1H), 2.93 (ddd, 1H), 2.85 (dd, 1H), 2.69 (dd, 1H), 2.41 (s, 3H); LC-MS (ESI) m/z 367.1 [M+H]⁺.

### Example 203: N-(((3R)-6-Fluoro-2-methyl-1,2,3,4-tetrahydroisoquinolin-3-yl)methyl)-3-(3-fluorophenyl)propanamide (203)

The compound was prepared by general procedure i1 with Pix (93 mg, 0.479 mmol), 3-(3-fluorophenyl) propanoic acid (76.5 mg, 0.455 mmol), 1-(3-dimethylaminopropyl)-3-ethylcarbodiimide hydrochloride (138 mg, 0.718 mmol), 1-hydroxybenzotriazole hydrate (97 mg, 0.718 mmol) and N,N-diisopropylethylamine (290 μL, 1.68 mmol) in dichloromethane (9.5 mL). The crude was purified by flash column chromatography (SiO₂, 3% methanol in dichloromethane) to give 203 as a yellow oil (72 mg, 46%). ¹H NMR (600 MHz, DMSO-d₆) δ 7.81 (t, 1H), 7.29 (td, 1H), 7.07 (dd, 1H), 7.06-7.02 (m, 2H), 7.00-6.96 (m, 1H), 6.93 (td, 1H), 6.86 (dd, 1H), 3.71 (d, 1H), 3.48 (d, 1H), 3.31-3.27 (m overlapped with H₂O, 1H), 3.05-2.99 (m, 1H), 2.83 (t, 2H), 2.66 (dd, 1H), 2.63-2.58 (m, 1H), 2.53 (dd, 1H), 2.42 (t, 2H), 2.30 (s, 3H); LC-MS (ESI) m/z 345.2 [M+H]⁺.

### Example 204: N-(((3R)-6-Fluoro-2-methyl-1,2,3,4-tetrahydroisoquinolin-3-yl)methyl)-1H-indene-2-carboxamide (204)

The compound was prepared by general procedure i1 with Pix (93 mg, 0.479 mmol), 1H-indene-2-carboxylic acid (73 mg, 0.455 mmol), 1-(3-dimethylaminopropyl)-3-ethylcarbodiimide hydrochloride (138 mg, 0.718 mmol), 1-hydroxybenzotriazole hydrate (97 mg, 0.718 mmol) and N,N-diisopropylethylamine (290 μL, 1.68 mmol) in dichloromethane (9.5 mL). The crude was purified by flash column chromatography (SiO₂, 3% methanol in dichloromethane) to give 204 as a brown syrup (89 mg, 58%). ¹H NMR (600 MHz, DMSO-d₆) δ 8.12 (br t, 1H), 7.57-7.50 (m, 3H), 7.33-7.26 (m, 2H), 7.10 (dd, 1H), 6.98-6.91 (m, 2H), 3.76 (d, 1H), 3.65 (d, 2H), 3.56 (d, 1H), 3.50 (dt, 1H), 3.15 (ddd, 1H), 2.92-2.86 (m, 1H), 2.84 (dd, 1H), 2.69 (dd, 1H), 2.41 (s, 3H); LC-MS (ESI) m/z 337.1 [M+H]⁺.

### Example 205: 4-Ethyl-N-(((3S)-6-fluoro-2-methyl-1,2,3,4-tetrahydroisoquinolin-3-yl)methyl)benzamide (205)

The compound was prepared by general procedure i1 with eix (77.5 mg, 0.399 mmol), 4-ethylbenzoic acid (57 mg, 0.379 mmol), 1-(3-dimethylaminopropyl)-3-ethylcarbodiimide hydrochloride (115 mg, 0.598 mmol), 1-hydroxybenzotriazole hydrate (81 mg, 0.598 mmol) and N,N-diisopropylethylamine (240 μL, 1.4 mmol) in dichloromethane (8 mL). The crude was purified by flash column chromatography (SiO₂, 3% methanol in dichloromethane) to give 205 as a beige solid (90 mg, 73%). ¹H NMR (600 MHz, DMSO-d₆) δ 8.35 (br t, 1H), 7.76 (d, 2H), 7.29 (d, 2H), 7.10 (dd, 1H), 6.97-6.91 (m, 2H), 3.75 (d, 1H), 3.58-3.51 (m, 2H), 3.18 (ddd, 1H), 2.91 (ddd, 1H), 2.83 (dd, 1H), 2.71-2.62 (m, 3H), 2.41 (s, 3H), 1.19 (t, 3H); LC-MS (ESI) m/z 327.1 [M+H]⁺.

### Example 206: N-(((3S)-6-Fluoro-2-methyl-1,2,3,4-tetrahydroisoquinolin-3-yl)methyl)-4-(trifluoromethyl)benzamide (206)

The compound was prepared by general procedure i1 with eix (77.5 mg, 0.399 mmol), 4-(trifluoromethyl)benzoic acid (72 mg, 0.379 mmol), 1-(3-dimethylaminopropyl)-3-ethylcarbodiimide hydrochloride (115 mg, 0.598 mmol), 1-hydroxybenzotriazole hydrate (81 mg, 0.598 mmol) and N,N-diisopropylethylamine (240 μL, 1.4 mmol) in dichloromethane (8 mL). The crude was purified by flash column chromatography (SiO₂, 3% methanol in dichloromethane) to give 206 as a white solid (112 mg, 81%). ¹H NMR (600 MHz, DMSO-d₆) δ 8.68 (br t, 1H), 8.03 (d, 2H), 7.85 (d, 2H), 7.14-7.08 (m, 1H), 6.98-6.91 (m, 2H), 3.76 (d, 1H), 3.61-3.54 (m, 2H), 3.26-3.19 (m, 1H), 2.97-2.91 (m, 1H), 2.85 (dd, 1H), 2.69 (dd, 1H), 2.41 (s, 3H); LC-MS (ESI) m/z 367.1 [M+H]⁺.

### Example 207: N-(((3S)-6-Fluoro-2-methyl-1,2,3,4-tetrahydroisoquinolin-3-yl)methyl)-3-(3-fluorophenyl)propanamide (207)

The compound was prepared by general procedure i1 with eix (77.5 mg, 0.399 mmol), 3-(3-fluorophenyl) propanoic acid (64 mg, 0.379 mmol), 1-(3-dimethylaminopropyl)-3-ethylcarbodiimide hydrochloride (115 mg, 0.598 mmol), 1-hydroxybenzotriazole hydrate (81 mg, 0.598 mmol) and N,N-diisopropylethylamine (240 μL, 1.4 mmol) in dichloromethane (8 mL). The crude was purified by flash column chromatography (SiO₂, 3% methanol in dichloromethane) to give 207 as a brown solid (75 mg, 57%). ¹H NMR (600 MHz, DMSO-d₆) δ 7.81 (br t, 1H), 7.29 (td, 1H), 7.07 (dd, 1H), 7.06-7.02 (m, 2H), 7.00-6.96 (m, 1H), 6.93 (td, 1H), 6.86 (dd, 1H), 3.71 (d, 1H), 3.48 (d, 1H), 3.29 (dd overlapped with H₂O, 1H), 3.02 (dt, 1H), 2.83 (t, 2H), 2.66 (dd, 1H), 2.64-2.58 (m, 1H), 2.53 (dd, 1H), 2.42 (t, 2H), 2.30 (s, 3H); LC-MS (ESI) m/z 345.2 [M+H]⁺.

### Example 208: N-(((3S)-6-Fluoro-2-methyl-1,2,3,4-tetrahydroisoquinolin-3-yl)methyl)-2,3-dihydro-1H-indene-2-carboxamide (208)

The compound was prepared by general procedure i1 with eix (77.5 mg, 0.399 mmol), 2-indanecarboxylic acid (61.5 mg, 0.379 mmol), 1-(3-dimethylaminopropyl)-3-ethylcarbodiimide hydrochloride (115 mg, 0.598 mmol), 1-hydroxybenzotriazole hydrate (81 mg, 0.598 mmol) and N,N-diisopropylethylamine (240 μL, 1.4 mmol) in dichloromethane (8 mL). The crude was purified by flash column chromatography (SiO₂, 3% methanol in dichloromethane) to give 208 as a white solid (81 mg, 63%). ¹H NMR (600 MHz, DMSO-d₆) δ 7.94 (br t, 1H), 7.20-7.16 (m, 2H), 7.13-7.10 (m, 2H), 7.09 (dd, 1H), 6.97-6.91 (m, 2H), 3.74 (d, 1H), 3.52 (d, 1H), 3.38 (dt, 1H), 3.22 (quin., 1H), 3.09 (dt, 1H), 3.06-3.02 (m, 4H), 2.79 (dd, 1H), 2.74-2.69 (m, 1H), 2.66 (dd, 1H), 2.36 (s, 3H); LC-MS (ESI) m/z 339.2 [M+H]⁺.

### Example 209: N-(((3S)-6-Fluoro-2-methyl-1,2,3,4-tetrahydroisoquinolin-3-yl)methyl)-1,2,3,4-tetrahydronaphthalene-2-carboxamide (209)

The compound was prepared by general procedure i1 with eix (77.5 mg, 0.399 mmol), 1,2,3,4-tetrahydronaphthalene-2-carboxylic acid (67 mg, 0.379 mmol), 1-(3-dimethylaminopropyl)-3-ethylcarbodiimide hydrochloride (115 mg, 0.598 mmol), 1-hydroxybenzotriazole hydrate (81 mg, 0.598 mmol) and N,N-diisopropylethylamine (240 μL, 1.4 mmol) in dichloromethane (8 mL). The crude was purified by flash column chromatography (SiO₂, 3% methanol in dichloromethane) to give 209 as a yellow syrup (95 mg, 71%). ¹H NMR (600 MHz, DMSO-d₆) δ 7.88 (br t, 1H), 7.09 (dd, 1H), 7.08-7.02 (m, 4H), 6.97-6.91 (m, 2H), 3.74 (d, 1H), 3.51 (d, 1H), 3.42-3.35 (m overlapped with H₂O, 1H), 3.13-3.03 (m, 1H), 2.87-2.69 (m, 6H), 2.66 (dd, 1H), 2.58-2.51 (m, 1H), 2.36 (s, 3H), 1.97-1.90 (m, 1H), 1.72-1.63 (m, 1H); LC-MS (ESI) m/z 353.2 [M+H]⁺.

### Example 210: trans-N-(((3S)-6-Fluoro-2-methyl-1,2,3,4-tetrahydroisoquinolin-3-yl)methyl)-2-phenylcyclopropane-1-carboxamide (210)

The compound was prepared by general procedure i1 with eix (77.5 mg, 0.399 mmol), trans-2-phenyl-1-cyclopropanecarboxylic acid (61.5 mg, 0.379 mmol), 1-(3-dimethylaminopropyl)-3-ethylcarbodiimide hydrochloride (115 mg, 0.598 mmol), 1-hydroxybenzotriazole hydrate (81 mg, 0.598 mmol) and N,N-diisopropylethylamine (240 μL, 1.4 mmol) in dichloromethane (8 mL). The crude was purified by flash column chromatography (SiO₂, 3% methanol in dichloromethane) to give 210 as a yellow syrup (31 mg, 24%). ¹H NMR (600 MHz, DMSO-d₆) δ 8.11 (br t, 1H), 7.26 (td, 2H), 7.19-7.15 (m, 1H), 7.13-7.10 (m, 2H), 7.08 (dd, 1H), 6.96-6.90 (m, 2H), 3.73 (d, 1H), 3.51 (d, 1H), 3.41-3.35 (m overlapped with H₂O, 1H), 3.15-3.07 (m, 1H), 2.76 (d, 1H), 2.71-2.62 (m, 2H), 2.34 (s, 1.5H), 2.33 (s, 1H), (d, 3H), 2.25-2.20 (m, 1H), 1.97 (ddd, 1H), 1.37-1.33 (m, 1H), 1.20-1.15 (m, 1H); LC-MS (ESI) m/z 339.2 [M+H]⁺.

### Example 211: N-(((3S)-6-Fluoro-2-methyl-1,2,3,4-tetrahydroisoquinolin-3-yl)methyl)-1H-indene-2-carboxamide (211)

The compound was prepared by general procedure i1 with eix (77.5 mg, 0.399 mmol), 1H-indene-2-carboxylic acid (60.7 mg, 0.379 mmol), 1-(3-dimethylaminopropyl)-3-ethylcarbodiimide hydrochloride (115 mg, 0.598 mmol), 1-hydroxybenzotriazole hydrate (81 mg, 0.598 mmol) and N,N-diisopropylethylamine (240 μL, 1.4 mmol) in dichloromethane (8 mL). The crude was purified by flash column chromatography (SiO₂, 3% methanol in dichloromethane) to give 211 as a brown syrup (69 mg, 54%). ¹H NMR (600 MHz, DMSO-d₆) δ 8.12 (br t, 1H), 7.56-7.50 (m, 3H), 7.33-7.27 (m, 2H), 7.10 (dd, 1H), 6.97-6.92 (m, 2H), 3.76 (d, 1H), 3.65 (d, 2H), 3.56 (d, 1H), 3.50 (dt, 1H), 3.15 (dt, 1H), 2.91-2.86 (m, 1H), 2.84 (dd, 1H), 2.69 (dd, 1H), 2.41 (s, 3H); LC-MS (ESI) m/z 337.2 [M+H]⁺.

### Example 212: N-((6-Chloro-2-methyl-1,2,3,4-tetrahydroisoquinolin-3-yl)methyl)-3-cyclohexylpropanamide (212)

The compound was prepared by general procedure i1 with Qix (39.5 mg, 0.187 mmol), 3-cyclohexylpropanoic acid (28.0 μL, 0.178 mmol), 1-(3-dimethylaminopropyl)-3-ethylcarbodiimide hydrochloride (54 mg, 0.281 mmol), 1-hydroxybenzotriazole hydrate (38 mg, 0.281 mmol) and N,N-diisopropylethylamine (110 μL, 0.656 mmol) in dichloromethane (3.7 mL). The crude was purified by flash column chromatography (SiO₂, 3-5% methanol in dichloromethane) to give 212 as a white solid (20 mg, 33%). ¹H NMR (600 MHz, DMSO-d₆) δ 7.75 (br t, 1H), 7.16 (dd, 1H), 7.13 (d, 1H), 7.08 (d, 1H), 3.73 (d, 1H), 3.51 (d, 1H), 3.30-3.27 (m, 1H), 3.04-2.98 (m, 1H), 2.75 (dd, 1H), 2.68 (ddd, 1H), 2.61 (dd, 1H), 2.34 (s, 3H), 2.08 (t, 2H), 1.70-1.57 (m, 5H), 1.38 (q, 2H), 1.21-1.08 (m, 4H), 0.88-0.79 (m, 2H); LC-MS (ESI) m/z 349.2 [M+H]⁺.

### Example 213: N-((6-Chloro-2-methyl-1,2,3,4-tetrahydroisoquinolin-3-yl)methyl)-3-phenylpropanamide (213)

The compound was prepared by general procedure i1 with Qix (76 mg, 0.361 mmol), 3-phenylpropionic acid (51 mg, 0.343 mmol), 1-(3-dimethylaminopropyl)-3-ethylcarbodiimide hydrochloride (104 mg, 0.541 mmol), 1-hydroxybenzotriazole hydrate (73 mg, 0.541 mmol) and N,N-diisopropylethylamine (220 μL, 1.26 mmol) in dichloromethane (7.2 mL). The crude was purified by flash column chromatography (SiO₂, 3% methanol in dichloromethane) to give 213 as a white solid (55 mg, 47%). ¹H NMR (600 MHz, DMSO-d₆) δ 7.79 (br t, 1H), 7.28-7.18 (m, 4H), 7.18-7.14 (m, 2H), 7.10 (d, 1H), 7.07 (d, 1H), 3.71 (d, 1H), 3.49 (d, 1H), 3.30-3.27 (m, 1H), 3.04-2.97 (m, 1H), 2.81 (t, 2H), 2.69-2.60 (m, 2H), 2.56-2.51 (m, 1H), 2.40 (t, 2H), 2.30 (s, 3H); LC-MS (ESI) m/z 343.1 [M+H]⁺.

### Example 214: N-((6-Chloro-2-methyl-1,2,3,4-tetrahydroisoquinolin-3-yl)methyl)-1,2,3,4-tetrahydronaphthalene-2-carboxamide (214)

The compound was prepared by general procedure i1 with Qix (39.5 mg, 0.187 mmol), 1,2,3,4-tetrahydronaphthalene-2-carboxylic acid (31 mg, 0.178 mmol), 1-(3-dimethylaminopropyl)-3-ethylcarbodiimide hydrochloride (54 mg, 0.281 mmol), 1-hydroxybenzotriazole hydrate (38 mg, 0.281 mmol) and N,N-diisopropylethylamine (110 μL, 0.656 mmol) in dichloromethane (3.7 mL). The crude was purified by flash column chromatography (SiO₂, 3% methanol in dichloromethane) to give 214 as a brown syrup (23 mg, 35%). ¹H NMR (600 MHz, DMSO-d₆) δ 7.87 (br t, 1H), 7.19-7.15 (m, 2H), 7.10-7.04 (m, 5H), 3.74 (d, 1H), 3.53 (d, 1H), 3.41-3.35 (m, 1H), 3.11-3.02 (m, 1H), 2.86-2.62 (m, 7H), 2.57-2.51 (m, 1H), 2.36 (s, 3H), 1.96-1.90 (m, 1H), 1.72-1.63 (m, 1H); LC-MS (ESI) m/z 369.1 [M+H]⁺.

### Example 215: N-((6-Chloro-2-methyl-1,2,3,4-tetrahydroisoquinolin-3-yl)methyl)-2,3-dihydro-1H-indene-2-carboxamide (215)

The compound was prepared by general procedure i1 with Qix (39.5 mg, 0.187 mmol), 2-indanecarboxylic acid (29 mg, 0.178 mmol), 1-(3-dimethylaminopropyl)-3-ethylcarbodiimide hydrochloride (54 mg, 0.281 mmol), 1-hydroxybenzotriazole hydrate (38 mg, 0.281 mmol) and N,N-diisopropylethylamine (110 μL, 0.656 mmol) in dichloromethane (3.7 mL). The crude was purified by flash column chromatography (SiO₂, 3% methanol in dichloromethane) to give 215 as a brown syrup (23 mg, 37%). ¹H NMR (600 MHz, DMSO-d₆) δ 7.93 (br t, 1H), 7.20-7.15 (m, 4H), 7.13-7.10 (m, 2H), 7.10-7.08 (m, 1H), 3.75 (d, 1H), 3.53 (d, 1H), 3.40-3.36 (m, 1H), 3.21 (quin., 1H), 3.08 (dd, 1H), 3.06-3.01 (m, 4H), 2.79 (dd, 1H), 2.74 (ddd, 1H), 2.65 (dd, 1H), 2.36 (s, 3H); LC-MS (ESI) m/z 355.1 [M+H]⁺.

### Example 216: N-((6-Chloro-2-methyl-1,2,3,4-tetrahydroisoquinolin-3-yl)methyl)-1H-indene-2-carboxamide (216)

The compound was prepared by general procedure i1 with Qix (39.5 mg, 0.187 mmol), 1H-indene-2-carboxylic acid (28.5 mg, 0.178 mmol), 1-(3-dimethylaminopropyl)-3-ethylcarbodiimide hydrochloride (54 mg, 0.281 mmol), 1-hydroxybenzotriazole hydrate (38 mg, 0.281 mmol) and N,N-diisopropylethylamine (110 μL, 0.656 mmol) in dichloromethane (3.7 mL). The crude was purified by flash column chromatography (SiO₂, 3% methanol in dichloromethane) to give 216 as a yellow syrup (23 mg, 37%). ¹H NMR (600 MHz, DMSO-d₆) δ 8.12 (br t, 1H), 7.56-7.54 (m, 1H), 7.54-7.50 (m, 2H), 7.33-7.27 (m, 2H), 7.19-7.16 (m, 2H), 7.10 (dd, 1H), 3.76 (d, 1H), 3.64 (d, 2H), 3.58 (d, 1H), 3.49 (dt, 1H), 3.13 (ddd, 1H), 2.93-2.87 (m, 1H), 2.84 (dd, 1H), 2.69 (dd, 1H), 2.41 (s, 3H); LC-MS (ESI) m/z 353.2 [M+H]⁺.

### Example 217: N-(((3R)-6-Chloro-2-methyl-1,2,3,4-tetrahydroisoquinolin-3-yl)methyl)-4-ethylbenzamide (217)

The compound was prepared by general procedure i1 with Rix (61 mg, 0.29 mmol), 4-ethylbenzoic acid (41 mg, 0.275 mmol), 1-(3-dimethylaminopropyl)-3-ethylcarbodiimide hydrochloride (83 mg, 0.434 mmol), 1-hydroxybenzotriazole hydrate (59 mg, 0.434 mmol) and N,N-diisopropylethylamine (180 μL, 1.01 mmol) in dichloromethane (6 mL). The crude was purified by flash column chromatography (SiO₂, 3% methanol in dichloromethane) to give 217 as a beige solid (84 mg, 68%). ¹H NMR (600 MHz, DMSO-d₆) δ 8.34 (br t, 1H), 7.75 (d, 2H), 7.29 (d, 2H), 7.19-7.15 (m, 2H), 7.11-7.08 (m, 1H), 3.76 (d, 1H), 3.57 (d, 1H), 3.53 (dt, 1H), 3.16 (ddd, 1H), 2.96-2.90 (m, 1H), 2.83 (dd, 1H), 2.71-2.62 (m, 3H), 2.41 (s, 3H), 1.19 (t, 3H); LC-MS (ESI) m/z 343.1 [M+H]⁺.

### Example 218: N-(((3R)-6-Chloro-2-methyl-1,2,3,4-tetrahydroisoquinolin-3-yl)methyl)-4-(ethylsulfanyl)benzamide (218)

The compound was prepared by general procedure i1 with Rix (61 mg, 0.29 mmol), 4-(ethylsulfanyl)benzoic acid (50 mg, 0.275 mmol), 1-(3-dimethylaminopropyl)-3-ethylcarbodiimide hydrochloride (83 mg, 0.434 mmol), 1-hydroxybenzotriazole hydrate (59 mg, 0.434 mmol) and N,N-diisopropylethylamine (180 μL, 1.01 mmol) in dichloromethane (6 mL). The crude was purified by flash column chromatography (SiO₂, 3% methanol in dichloromethane) to give 218 as a beige solid (79 mg, 77%). ¹H NMR (600 MHz, DMSO-d₆) δ 8.39 (br t, 1H), 7.77 (d, 2H), 7.35 (d, 2H), 7.18-7.15 (m, 2H), 7.11-7.07 (m, 1H), 3.75 (d, 1H), 3.57 (d, 1H), 3.53 (dt, 1H), 3.16 (ddd, 1H), 3.05 (q, 2H), 2.95-2.90 (m, 1H), 2.83 (dd, 1H), 2.67 (dd, 1H), 2.41 (s, 3H), 1.26 (t, 3H); LC-MS (ESI) m/z 375.1 [M+H]⁺.

### Example 219: 3,4-Dichloro-N-(((3R)-6-chloro-2-methyl-1,2,3,4-tetrahydroisoquinolin-3-yl)methyl)benzamide (219)

The compound was prepared by general procedure i1 with Rix (61 mg, 0.29 mmol), 3,4-dichlorobenzoic acid (53 mg, 0.275 mmol), 1-(3-dimethylaminopropyl)-3-ethylcarbodiimide hydrochloride (83 mg, 0.434 mmol), 1-hydroxybenzotriazole hydrate (59 mg, 0.434 mmol) and N,N-diisopropylethylamine (180 μL, 1.01 mmol) in dichloromethane (6 mL). The crude was purified by flash column chromatography (SiO₂, 3% methanol in dichloromethane) to give 219 as a beige solid (87 mg, 82%). ¹H NMR (600 MHz, DMSO-d₆) δ 8.63 (br t, 1H), 8.05 (d, 1H), 7.81 (dd, 1H), 7.75 (d, 1H), 7.19-7.15 (m, 2H), 7.09 (d, 1H), 3.75 (d, 1H), 3.58 (d, 1H), 3.54 (dt, 1H), 3.17 (ddd, 1H), 2.96-2.90 (m, 1H), 2.84 (dd, 1H), 2.68 (dd, 1H), 2.40 (s, 3H); LC-MS (ESI) m/z 383.0 [M+H]⁺.

### Example 220: N-(((3R)-6-Chloro-2-methyl-1,2,3,4-tetrahydroisoquinolin-3-yl)methyl)-1-benzothiophene-2-carboxamide (220)

The compound was prepared by general procedure i1 with Rix (47.5 mg, 0.225 mmol), 1-benzothiophene-2-carboxylic acid (38 mg, 0.214 mmol), 1-(3-dimethylaminopropyl)-3-ethylcarbodiimide hydrochloride (65 mg, 0.338 mmol), 1-hydroxybenzotriazole hydrate (46 mg, 0.338 mmol) and N,N-diisopropylethylamine (140 μL, 0.789 mmol) in dichloromethane (4.5 mL). The crude was purified by flash column chromatography (SiO₂, 3% methanol in dichloromethane) to give 220 as a beige solid (43 mg, 54%). ¹H NMR (600 MHz, DMSO-d₆) δ 8.73 (br t, 1H), 8.09 (d, 1H), 8.03-8.00 (m, 1H), 7.96-7.93 (m, 1H), 7.48-7.42 (m, 2H), 7.20-7.16 (m, 2H), 7.11 (d, 1H), 3.77 (d, 1H), 3.60 (d, 1H), 3.55 (dt, 1H), 3.19 (ddd, 1H), 2.99-2.93 (m, 1H), 2.86 (dd, 1H), 2.71 (dd, 1H), 2.42 (s, 3H); LC-MS (ESI) m/z 371.1 [M+H]⁺.

### Example 221: N-(((3R)-6-Chloro-2-methyl-1,2,3,4-tetrahydroisoquinolin-3-yl)methyl)-1-benzothiophene-3-carboxamide (221)

The compound was prepared by general procedure i1 with Rix (47.5 mg, 0.225 mmol), 1-benzothiophene-3-carboxylic acid (38 mg, 0.214 mmol), 1-(3-dimethylaminopropyl)-3-ethylcarbodiimide hydrochloride (65 mg, 0.338 mmol), 1-hydroxybenzotriazole hydrate (46 mg, 0.338 mmol) and N,N-diisopropylethylamine (140 μL, 0.789 mmol) in dichloromethane (4.5 mL). The crude was purified by flash column chromatography (SiO₂, 3% methanol in dichloromethane) to give 221 as a white solid (49 mg, 62%). ¹H NMR (600 MHz, DMSO-d₆) δ 8.45-8.40 (m, 2H), 8.31 (s, 1H), 8.03 (d, 1H), 7.47-7.39 (m, 2H), 7.20-7.16 (m, 2H), 7.11 (d, 1H), 3.78 (d, 1H), 3.62-3.54 (m, 2H), 3.21 (ddd, 1H), 2.95 (ddd, 1H), 2.88 (dd, 1H), 2.73 (dd, 1H), 2.44 (s, 3H); LC-MS (ESI) m/z 371.1 [M+H]⁺.

### Example 222: N-(((3R)-6-Chloro-2-methyl-1,2,3,4-tetrahydroisoquinolin-3-yl)methyl)-1-benzothiophene-4-carboxamide (222)

The compound was prepared by general procedure i1 with Rix (47.5 mg, 0.225 mmol), 1-benzothiophene-4-carboxylic acid (38 mg, 0.214 mmol), 1-(3-dimethylaminopropyl)-3-ethylcarbodiimide hydrochloride (65 mg, 0.338 mmol), 1-hydroxybenzotriazole hydrate (46 mg, 0.338 mmol) and N,N-diisopropylethylamine (140 μL, 0.789 mmol) in dichloromethane (4.5 mL). The crude was purified by flash column chromatography (SiO₂, 3% methanol in dichloromethane) to 222 as a white solid (49 mg, 62%). ¹H NMR (600 MHz, DMSO-d₆) δ 8.70 (br t, 1H), 8.06 (dd, 1H), 7.95 (d, 1H), 7.82 (d, 1H), 7.52-7.47 (m, 2H), 7.20-7.16 (m, 2H), 7.11 (d, 1H), 3.78 (d, 1H), 3.65-3.58 (m, 2H), 3.24 (ddd, 1H), 3.03-2.97 (m, 1H), 2.87 (dd, 1H), 2.72 (dd, 1H), 2.44 (s, 3H); LC-MS (ESI) m/z 371.1 [M+H]⁺.

### Example 223: N-(((3R)-6-Chloro-2-methyl-1,2,3,4-tetrahydroisoquinolin-3-yl)methyl)-1-benzothiophene-5-carboxamide (223)

The compound was prepared by general procedure i1 with Rix (47.5 mg, 0.225 mmol), 1-benzothiophene-5-carboxylic acid (38 mg, 0.214 mmol), 1-(3-dimethylaminopropyl)-3-ethylcarbodiimide hydrochloride (65 mg, 0.338 mmol), 1-hydroxybenzotriazole hydrate (46 mg, 0.338 mmol) and N,N-diisopropylethylamine (140 μL, 0.789 mmol) in dichloromethane (4.5 mL). The crude was purified by flash column chromatography (SiO₂, 3% methanol in dichloromethane) to give 223 as a white solid (39 mg, 49%). ¹H NMR (600 MHz, DMSO-d₆) 8.51 (br t, 1H), 8.35 (s, 1H), 8.08 (d, 1H), 7.86 (d, 1H), 7.82 (dd, 1H), 7.56 (dd, 1H), 7.20-7.15 (m, 2H), 7.10 (d, 1H), 3.77 (d, 1H), 3.62-3.55 (m, 2H), 3.22 (ddd, 1H), 3.00-2.94 (m, 1H), 2.86 (dd, 1H), 2.72 (dd, 1H), 2.43 (s, 3H); LC-MS (ESI) m/z 371.1 [M+H]⁺.

### Example 224: N-(((3R)-6-Chloro-2-methyl-1,2,3,4-tetrahydroisoquinolin-3-yl)methyl)-6-(trifluoromethyl)-1-benzothiophene-2-carboxamide (224)

The compound was prepared by general procedure i1 with Rix (47.5 mg, 0.225 mmol), 6-(tri-fluoromethyl)-1-benzothiophene-2-carboxylic acid (53 mg, 0.214 mmol), 1-(3-dimethylamino-propyl)-3-ethylcarbodiimide hydrochloride (65 mg, 0.338 mmol), 1-hydroxybenzotriazole hydrate (46 mg, 0.338 mmol) and N,N-diisopropylethylamine (140 μL, 0.789 mmol) in dichloromethane (4.5 mL). The crude was purified by flash column chromatography (SiO₂, 3% methanol in dichloromethane) to give 224 as an off-white solid (41 mg, 44%). ¹H NMR (600 MHz, DMSO-d₆) δ 8.88 (br t, 1H), 8.58-8.55 (m, 1H), 8.19 (s, 1H), 8.16 (d, 1H), 7.73 (dd, 1H), 7.20-7.16 (m, 2H), 7.11 (d, 1H), 3.77 (d, 1H), 3.60 (d, 1H), 3.58-3.54 (m, 1H), 3.21 (ddd, 1H), 3.00-2.94 (m, 1H), 2.87 (dd, 1H), 2.71 (dd, 1H), 2.42 (s, 3H); LC-MS (ESI) m/z 439.0 [M+H]⁺.

### Example 225: N-(((3R)-6-Chloro-2-methyl-1,2,3,4-tetrahydroisoquinolin-3-yl)methyl)-1H-indene-2-carboxamide (225)

The compound was prepared by general procedure i1 with Rix (61 mg, 0.29 mmol), 1H-indene-2-carboxylic acid (44 mg, 0.275 mmol), 1-(3-dimethylaminopropyl)-3-ethylcarbodiimide hydrochloride (83 mg, 0.434 mmol), 1-hydroxybenzotriazole hydrate (59 mg, 0.434 mmol) and N,N-diisopropylethylamine (180 μL, 1.01 mmol) in dichloromethane (6 mL). The crude was purified by flash column chromatography (SiO₂, 3% methanol in dichloromethane) to give 225 as a brown syrup (57 mg, 59%). ¹H NMR (600 MHz, DMSO-d₆) δ 8.12 (br t, 1H), 7.56-7.50 (m, 3H), 7.33-7.26 (m, 2H), 7.19-7.15 (m, 2H), 7.10 (d, 1H), 3.76 (d, 1H), 3.66-3.63 (m, 2H), 3.58 (d, 1H), 3.49 (dt, 1H), 3.13 (ddd, 1H), 2.93-2.87 (m, 1H), 2.84 (dd, 1H), 2.69 (dd, 1H), 2.41 (s, 3H); LC-MS (ESI) m/z 353.1 [M+H]⁺.

### Example 226: (2R)—N-(((3R)-6-Chloro-2-methyl-1,2,3,4-tetrahydroisoquinolin-3-yl)methyl)-1,2,3,4-tetrahydronaphthalene-2-carboxamide (226)

The compound was prepared by general procedure i1 with Rix (61 mg, 0.29 mmol), (2R)-1,2,3,4-tetrahydronaphthalene-2-carboxylic acid (48 mg, 0.275 mmol), 1-(3-dimethylaminopropyl)-3-ethylcarbodiimide hydrochloride (83 mg, 0.434 mmol), 1-hydroxybenzotriazole hydrate (59 mg, 0.434 mmol) and N,N-diisopropylethylamine (180 μL, 1.01 mmol) in dichloromethane (6 mL). The crude was purified by flash column chromatography (SiO₂, 3% methanol in dichloromethane) to give 226 as a white solid (61 mg, 60%). ¹H NMR (600 MHz, DMSO-d₆) δ 7.87 (br t, 1H), 7.19-7.15 (m, 2H), 7.11-7.02 (m, 5H), 3.75 (d, 1H), 3.53 (d, 1H), 3.38-3.33 (m, 1H), 3.08 (ddd, 1H), 2.86-2.69 (m, 6H), 2.65 (dd, 1H), 2.57-2.51 (m, 1H), 2.36 (s, 3H), 1.97-1.90 (m, 1H), 1.68 (dtd, 1H); LC-MS (ESI) m/z 369.1 [M+H]⁺.

### Example 227: (2S)—N-(((3R)-6-Chloro-2-methyl-1,2,3,4-tetrahydroisoquinolin-3-yl)methyl)-1,2,3,4-tetrahydronaphthalene-2-carboxamide (227)

The compound was prepared by general procedure i1 with Rix (61 mg, 0.29 mmol), (2S)-1,2,3,4-tetrahydronaphthalene-2-carboxylic acid (48 mg, 0.275 mmol), 1-(3-dimethylaminopropyl)-3-ethylcarbodiimide hydrochloride (83 mg, 0.434 mmol), 1-hydroxybenzotriazole hydrate (59 mg, 0.434 mmol) and N,N-diisopropylethylamine (180 μL, 1.01 mmol) in dichloromethane (6 mL). The crude was purified by flash column chromatography (SiO₂, 3% methanol in dichloromethane) to give 227 as a brown syrup (72 mg, 71%). ¹H NMR (600 MHz, DMSO-d₆) δ 7.88 (br t, 1H), 7.19-7.15 (m, 2H), 7.12-7.04 (m, 5H), 3.74 (d, 1H), 3.53 (d, 1H), 3.39 (dt, 1H), 3.05 (ddd, 1H), 2.87-2.69 (m, 6H), 2.65 (dd, 1H), 2.57-2.51 (m, 1H), 2.36 (s, 3H), 1.96-1.90 (m, 1H), 1.71-1.63 (m, 1H); LC-MS (ESI) m/z 369.2 [M+H]⁺.

### Example 228: N-(((3S)-6-Chloro-2-methyl-1,2,3,4-tetrahydroisoquinolin-3-yl)methyl)-4-ethylbenzamide (228)

The compound was prepared by general procedure i2 with fix (25 mg, 0.119 mmol), 4-ethyl-benzoyl chloride (17 μL, 0.113 mmol), and triethylamine (17 μL, 0.119 mmol) in dichloromethane (2.4 mL). The crude was purified by flash column chromatography (SiO₂, 3% methanol in dichloromethane) to give 228 as a beige solid (20 mg, 52%). ¹H NMR (600 MHz, CDCl₃) δ 7.68 (d, 2H), 7.24 (d, 2H), 7.11 (dd, 1H), 7.08 (s, 1H), 6.97 (d, 1H), 6.77 (br s, 1H), 3.85 (d, 1H), 3.69 (d, 1H), 3.64 (dt, 1H), 3.52 (ddd, 1H), 3.0-2.95 (m, 1H), 2.83 (dd, 1H), 2.77 (dd, 1H), 2.68 (q, 2H), 2.43 (s, 3H), 1.24 (t, 3H); LC-MS (ESI) m/z 343.2 [M+H]⁺.

### Example 229: N-(((3S)-6-Chloro-2-methyl-1,2,3,4-tetrahydroisoquinolin-3-yl)methyl)-4-(ethylsulfanyl)benzamide (229)

The compound was prepared by general procedure i1 with fix (35 mg, 0.166 mmol), 4-(ethylsulfanyl)benzoic acid (29 mg, 0.158 mmol), 1-(3-dimethylaminopropyl)-3-ethylcarbodiimide hydrochloride (48 mg, 0.249 mmol), 1-hydroxybenzotriazole hydrate (34 mg, 0.249 mmol) and N,N-diisopropylethylamine (100 μL, 0.581 mmol) in dichloromethane (3.3 mL). The crude was purified by flash column chromatography (SiO₂, 3% methanol in dichloromethane) to give 229 as a white solid (30 mg, 51%). ¹H NMR (600 MHz, CDCl₃) δ 7.66 (dd, 2H), 7.27 (dd, 2H), 7.11 (dd, 1H), 7.08 (s, 1H), 6.97 (d, 1H), 6.83 (br s, 1H), 3.84 (d, 1H), 3.69 (d, 1H), 3.63 (dt, 1H), 3.51 (ddd, 1H), 3.01-2.95 (m, 3H), 2.82 (dd, 1H), 2.76 (dd, 1H), 2.42 (s, 3H), 1.33 (t, 3H); LC-MS (ESI) m/z 375.1 [M+H]⁺.

### Example 230: 3,4-Dichloro-N-(((3S)-6-chloro-2-methyl-1,2,3,4-tetrahydroisoquinolin-3-yl)methyl)benzamide (230)

The compound was prepared by general procedure i1 with fix (35 mg, 0.166 mmol), 3,4-dichlorobenzoic acid (30 mg, 0.158 mmol), 1-(3-dimethylaminopropyl)-3-ethylcarbodiimide hydrochloride (48 mg, 0.249 mmol), 1-hydroxybenzotriazole hydrate (34 mg, 0.249 mmol) and N,N-diisopropylethylamine (100 μL, 0.581 mmol) in dichloromethane (3.3 mL). The crude was purified by flash column chromatography (SiO₂, 2% methanol in dichloromethane) to give 230 as a white solid (33 mg, 54%). ¹H NMR (600 MHz, CDCl₃) δ 7.82 (d, 1H), 7.57 (dd, 1H), 7.47 (d, 1H), 7.12 (dd, 1H), 7.08 (s, 1H), 6.98 (d, 1H), 6.93 (br s, 1H), 3.85 (d, 1H), 3.71 (d, 1H), 3.63 (dt, 1H), 3.49 (ddd, 1H), 3.05-2.99 (m, 1H), 2.84 (dd, 1H), 2.75 (dd, 1H), 2.43 (s, 3H); LC-MS (ESI) m/z 383.0 [M+H]⁺.

### Example 231: N-(((3S)-6-Chloro-2-methyl-1,2,3,4-tetrahydroisoquinolin-3-yl)methyl)-4-(trifluoromethyl)benzamide (231)

The compound was prepared by general procedure i1 with fix (25 mg, 0.119 mmol), 4-(trifluoromethyl)benzoic acid (21.4 mg, 0.113 mmol), 1-(3-dimethylaminopropyl)-3-ethylcarbodiimide hydrochloride (34 mg, 0.178 mmol), 1-hydroxybenzotriazole hydrate (24 mg, 0.178 mmol) and N,N-diisopropylethylamine (72.0 μL, 0.415 mmol) in dichloromethane (2.4 mL). The crude was purified by flash column chromatography (SiO₂, 2-4% methanol in dichloromethane) to give 231 as a white solid (24 mg, 56%). ¹H NMR (600 MHz, CDCl₃) δ 7.85 (d, 2H), 7.68 (d, 2H), 7.12 (dd, 1H), 7.09 (d, 1H), 6.98 (d, 1H), 6.85 (br s, 1H), 3.85 (d, 1H), 3.71 (d, 1H), 3.67 (dt, 1H), 3.51 (ddd, 1H), 3.05-2.99 (m, 1H), 2.85 (dd, 1H), 2.76 (dd, 1H), 2.43 (s, 3H); LC-MS (ESI) m/z 383.1 [M+H]⁺.

### Example 232: N-(((3S)-6-Chloro-2-methyl-1,2,3,4-tetrahydroisoquinolin-3-yl)methyl)-3-(3-fluorophenyl)propanamide (232)

The compound was prepared by general procedure i1 with fix (35 mg, 0.166 mmol), 3-(3-fluorophenyl) propionic acid (26.5 mg, 0.158 mmol), 1-(3-dimethylaminopropyl)-3-ethylcarbodiimide hydrochloride (48 mg, 0.249 mmol), 1-hydroxybenzotriazole hydrate (34 mg, 0.249 mmol) and N,N-diisopropylethylamine (100 μL, 0.581 mmol) in dichloromethane (3.3 mL). The crude was purified by flash column chromatography (SiO₂, 3-4% methanol in dichloromethane) to give 232 as a beige solid (24 mg, 59%). ¹H NMR (600 MHz, CDCl₃) δ 7.21 (td, 1H), 7.10 (dd, 1H), 7.05 (s, 1H), 6.97 (d, 1H), 6.94 (d, 1H), 6.90 (dt, 1H), 6.87 (td, 1H), 5.95 (br s, 1H), 3.75 (d, 1H), 3.60 (d, 1H), 3.40 (dt, 1H), 3.31 (dt, 1H), 2.96 (t, 2H), 2.80-2.74 (m, 1H), 2.68 (dd, 1H), 2.58 (dd, 1H), 2.49 (td, 2H), 2.30 (s, 3H); LC-MS (ESI) m/z 361.1 [M+H]⁺.

### Example 233: N-(((3S)-6-Chloro-2-methyl-1,2,3,4-tetrahydroisoquinolin-3-yl)methyl)-3-cyclohexylpropanamide (233)

The compound was prepared by general procedure i1 with fix (35 mg, 0.166 mmol), 3-cyclohexylpropanoic acid (25.0 μL, 0.158 mmol), 1-(3-dimethylaminopropyl)-3-ethylcarbodiimide hydrochloride (48 mg, 0.249 mmol), 1-hydroxybenzotriazole hydrate (34 mg, 0.249 mmol) and N,N-diisopropylethylamine (100 μL, 0.581 mmol) in dichloromethane (3.3 mL). The crude was purified by flash column chromatography (SiO₂, 3% methanol in dichloromethane) to give 233 as a white solid (33 mg, 60%). ¹H NMR (400 MHz, Acetone-d₆) δ 7.15-7.10 (m, 2H), 7.07 (d, 1H), 7.02 (br s, 1H), 3.81 (d, 1H), 3.57 (d, 1H), 3.46 (dt, 1H), 3.22 (dt, 1H), 2.86-2.67 (m overlapped with H₂O, 2H), 2.40 (s, 3H), 2.22-2.14 (m, 2H), 1.77-1.57 (m, 5H), 1.48 (dt, 2H), 1.37-1.07 (m, 5H), 0.96-0.81 (m, 2H); LC-MS (ESI) m/z 349.2 [M+H]⁺.

### Example 234: N-(((3S)-6-Chloro-2-methyl-1,2,3,4-tetrahydroisoquinolin-3-yl)methyl)-3-phenylpropanamide (234)

The compound was prepared by general procedure i1 with fix (35 mg, 0.166 mmol), 3-phenylpropionic acid (24 mg, 0.158 mmol), 1-(3-dimethylaminopropyl)-3-ethylcarbodiimide hydrochloride (48 mg, 0.249 mmol), 1-hydroxybenzotriazole hydrate (34 mg, 0.249 mmol) and N,N-diisopropylethylamine (100 μL, 0.581 mmol) in dichloromethane (3.3 mL). The crude was purified by flash column chromatography (SiO₂, 3% methanol in dichloromethane) to give 234 as a white solid (30 mg, 56%). ¹H NMR (600 MHz, CDCl₃) δ 7.29-7.26 (m overlapped with CDCl₃, 2H), 7.23-7.17 (m, 3H), 7.12 (dd, 1H), 7.06 (d, 1H), 6.95 (d, 1H), 6.02 (br s, 1H), 3.77 (d, 1H), 3.62 (d, 1H), 3.41 (dt, 1H), 3.32 (ddd, 1H), 2.97 (t, 2H), 2.82-2.76 (m, 1H), 2.68 (dd, 1H), 2.58 (dd, 1H), 2.54-2.49 (m, 2H), 2.32 (s, 3H); LC-MS (ESI) m/z 343.1 [M+H]⁺.

### Example 235: N-(((3S)-6-Chloro-2-methyl-1,2,3,4-tetrahydroisoquinolin-3-yl)methyl)-1,2,3,4-tetrahydronaphthalene-2-carboxamide (235)

The compound was prepared by general procedure i1 with fix (35 mg, 0.166 mmol), 1,2,3,4-tetrahydronaphthalene-2-carboxylic acid (28 mg, 0.158 mmol), 1-(3-dimethylaminopropyl)-3-ethylcarbodiimide hydrochloride (48 mg, 0.249 mmol), 1-hydroxybenzotriazole hydrate (34 mg, 0.249 mmol) and N,N-diisopropylethylamine (100 μL, 0.581 mmol) in dichloromethane (3.3 mL). The crude was purified by flash column chromatography (SiO₂, 3% methanol in dichloromethane) to give 235 as a colorless syrup (41 mg, 70%). ¹H NMR (400 MHz, CDCl₃) δ 7.14-7.04 (m, 6H), 6.96 (d, 1H), 6.26 (br s, 1H), 3.80 (dd, 1H), 3.64 (d, 1H), 3.50-3.35 (m, 2H), 3.02 (dd, 1H), 2.95-2.64 (m, 6H), 2.59-2.49 (m, 1H), 2.38 (s, 1.5H), 2.38 (s, 1.5H), 2.12-2.04 (m, 1H), 1.96-1.84 (m, 1H); LC-MS (ESI) m/z 369.1 [M+H]⁺.

### Example 236: trans-N-(((3S)-6-Chloro-2-methyl-1,2,3,4-tetrahydroisoquinolin-3-yl)methyl)-2-phenylcyclopropane-1-carboxamide (236)

The compound was prepared by general procedure i1 with fix (99 mg, 0.47 mmol), trans-2-phenyl-1-cyclopropanecarboxylic acid (69 mg, 0.423 mmol), 1-(3-dimethylaminopropyl)-3-ethylcarbodiimide hydrochloride (135 mg, 0.705 mmol), 1-hydroxybenzotriazole hydrate (95 mg, 0.705 mmol) and N,N-diisopropylethylamine (290 μL, 1.64 mmol) in dichloromethane (9.4 mL). The crude was purified by flash column chromatography (SiO₂, 3% methanol in dichloromethane) to give 236 as a white solid (28 mg, 19%). ¹H NMR (600 MHz, DMSO-d₆) δ 8.10 (br q, 1H), 7.28-7.24 (m, 2H), 7.19-7.09 (m, 5H), 7.08 (d, 1H), 3.73 (d, 1H), 3.52 (d, 1H), 3.40-3.32 (m, 1H), 3.09 (ddt, 1H), 2.80-2.73 (m, 1H), 2.73-2.68 (m, 1H), 2.68-2.60 (m, 1H), 2.34 (s, 1.5H), 2.33 (s, 1.5H), 2.23 (dtd, 1H), 1.99-1.93 (m, 1H), 1.34 (dddd, 1H), 1.17 (dddd, 1H); LC-MS (ESI) m/z 355.1 [M+H]⁺.

### Example 237: N-(((3S)-6-Chloro-2-methyl-1,2,3,4-tetrahydroisoquinolin-3-yl)methyl)-2,3-dihydro-1H-indene-2-carboxamide (237)

The compound was prepared by general procedure i1 with fix (35 mg, 0.166 mmol), 2-indanecarboxylic acid (26 mg, 0.158 mmol), 1-(3-dimethylaminopropyl)-3-ethylcarbodiimide hydrochloride (48 mg, 0.249 mmol), 1-hydroxybenzotriazole hydrate (34 mg, 0.249 mmol) and N,N-diisopropylethylamine (100 μL, 0.581 mmol) in dichloromethane (3.3 mL). The crude was purified by flash column chromatography (SiO₂, 3% methanol in dichloromethane) to give 237 as a beige solid (40 mg, 71%). ¹H NMR (400 MHz, CDCl₃) δ 7.22-7.13 (m, 4H), 7.12 (dd, 1H), 7.08 (d, 1H), 6.96 (d, 1H), 6.19 (br s, 1H), 3.81 (d, 1H), 3.65 (d, 1H), 3.48 (dt, 1H), 3.39 (ddd, 1H), 3.29-3.10 (m, 5H), 2.91-2.83 (m, 1H), 2.77 (dd, 1H), 2.69 (dd, 1H), 2.38 (s, 3H); LC-MS (ESI) m/z 355.1 [M+H]⁺.

### Example 238: (2R)—N-(((3S)-6-Chloro-2-methyl-1,2,3,4-tetrahydroisoquinolin-3-yl)methyl)-1,2,3,4-tetrahydronaphthalene-2-carboxamide (238)

The compound was prepared by general procedure i1 with fix (35 mg, 0.166 mmol), (2R)-1,2,3,4-tetrahydronaphthalene-2-carboxylic acid (28 mg, 0.158 mmol), 1-(3-dimethylaminopropyl)-3-ethylcarbodiimide hydrochloride (48 mg, 0.249 mmol), 1-hydroxybenzotriazole hydrate (34 mg, 0.249 mmol) and N,N-diisopropylethylamine (100 μL, 0.581 mmol) in dichloromethane (3.3 mL). The crude was purified by flash column chromatography (SiO₂, 3% methanol in dichloromethane) to give 238 as a white syrup (26 mg, 45%). ¹H NMR (600 MHz, DMSO-d₆) δ 7.88 (br s, 1H), 7.17 (d, 2H), 7.09 (d, 1H), 7.08-7.03 (m, 4H), 3.76 (d, 1H), 3.54 (d, 1H), 3.39 (dt, 1H), 3.12-3.01 (m, 1H), 2.87-2.62 (m, 7H), 2.57-2.51 (m, 1H), 2.37 (s, 3H), 1.97-1.90 (m, 1H), 1.72-1.63 (m, 1H); LC-MS (ESI) m/z 369.1 [M+H]⁺.

### Example 239: (2S)—N-(((3S)-6-Chloro-2-methyl-1,2,3,4-tetrahydroisoquinolin-3-yl)methyl)-1,2,3,4-tetrahydronaphthalene-2-carboxamide (239)

The compound was prepared by general procedure i1 with fix (99 mg, 0.47 mmol), (2S)-1,2,3,4-tetrahydronaphthalene-2-carboxylic acid (75 mg, 0.423 mmol), 1-(3-dimethylaminopropyl)-3-ethylcarbodiimide hydrochloride (135 mg, 0.705 mmol), 1-hydroxybenzotriazole hydrate (95 mg, 0.705 mmol) and N,N-diisopropylethylamine (290 μL, 1.65 mmol) in dichloromethane (9.4 mL). The crude was purified by flash column chromatography (SiO₂, 3% methanol in dichloromethane) to give 239 as a white syrup (64 mg, 41%). ¹H NMR (600 MHz, DMSO-d₆) δ 7.87 (br t, 1H), 7.18-7.15 (m, 2H), 7.11-7.03 (m, 5H), 3.75 (d, 1H), 3.53 (d, 1H), 3.36 (dt overlapped with H₂O, 1H), 3.08 (ddd, 1H), 2.86-2.69 (m, 6H), 2.65 (dd, 1H), 2.54 (tdd overlapped with DMSO-d₆, 1H), 2.36 (s, 3H), 1.96-1.90 (m, 1H), 1.68 (dtd, 1H); LC-MS (ESI) m/z 369.2 [M+H]⁺.

### Example 240: N-((6-Bromo-2-methyl-1,2,3,4-tetrahydroisoquinolin-3-yl)methyl)-2,3-dihydro-1H-indene-2-carboxamide (240)

The compound was prepared by general procedure i1 with gix (233 mg, 0.913 mmol), 2-indanecarboxylic acid (133 mg, 0.822 mmol), 1-(3-dimethylaminopropyl)-3-ethylcarbodiimide hydrochloride (262 mg, 1.37 mmol), 1-hydroxybenzotriazole hydrate (185 mg, 1.37 mmol) and N,N-diisopropylethylamine (560 μL, 3.20 mmol) in dichloromethane (9 mL). The crude was purified by flash column chromatography (SiO₂, 3% methanol in dichloromethane) to give 240 as a yellow syrup (223 mg, 68%). ¹H NMR (600 MHz, DMSO-d₆) δ 7.93 (br t, 1H), 7.32-7.28 (m, 2H), 7.20-7.16 (m, 2H), 7.13-7.10 (m, 2H), 7.03 (d, 1H), 3.73 (d, 1H), 3.51 (d, 1H), 3.40-3.35 (m, 1H), 3.20 (quin., 1H), 3.10-3.00 (m, 5H), 2.79 (dd, 1H), 2.76-2.71 (m, 1H), 2.65 (dd, 1H), 2.36 (s, 3H); LC-MS (ESI) m/z 399.1 [M+H]⁺.

### Example 241: N-((6-Bromo-2-methyl-1,2,3,4-tetrahydroisoquinolin-3-yl)methyl)-1, 2, 3, 4-tetrahydronaphthalene-2-carboxamide (241)

The compound was prepared by general procedure i1 with gix (233 mg, 0.913 mmol), 2-indanecarboxylic acid (133 mg, 0.822 mmol), 1-(3-dimethylaminopropyl)-3-ethylcarbodiimide hydrochloride (145 mg, 1.37 mmol), 1-hydroxybenzotriazole hydrate (185 mg, 1.37 mmol) and N,N-diisopropylethylamine (560 μL, 3.2 mmol) in dichloromethane (9 mL). The crude was purified by flash column chromatography (SiO₂, 3% methanol in dichloromethane) to give 241 as a yellow syrup (269 mg, 79%). ¹H NMR (600 MHz, DMSO-d₆) δ 7.89-7.84 (m, 1H), 7.32-7.28 (m, 2H), 7.08-7.04 (m, 4H), 7.03 (d, 1H), 3.72 (d, 1H), 3.51 (d, 1H), 3.41-3.33 (m, 1H), 3.10-3.01 (m, 1H), 2.86-2.69 (m, 6H), 2.65 (dd, 1H), 2.57-2.51 (m, 1H), 2.36 (s, 3H), 1.96-1.89 (m, 1H), 1.72-1.62 (m, 1H); LC-MS (ESI) m/z 413.1 [M+H]⁺.

### Example 242: N-(((3R)-6-Bromo-2-methyl-1,2,3,4-tetrahydroisoquinolin-3-yl)methyl)-1,2,3,4-tetrahydronaphthalene-2-carboxamide (242)

The compound was prepared by general procedure i1 with Six (50 mg, 0.196 mmol), 1,2,3,4-tetrahydronaphthalene-2-carboxylic acid (33 mg, 0.186 mmol), 1-(3-dimethylaminopropyl)-3-ethylcarbodiimide hydrochloride (56 mg, 0.294 mmol), 1-hydroxybenzotriazole hydrate (40 mg, 0.294 mmol) and N,N-diisopropylethylamine (120 μL, 0.686 mmol) in dichloromethane (4 mL). The crude was purified by flash column chromatography (SiO₂, 3% methanol in dichloromethane) to give 242 as an off-white syrup (45 mg, 59%). ¹H NMR (600 MHz, DMSO-d₆) δ 7.88 (br t, 1H), 7.32-7.27 (m, 2H), 7.08-7.04 (m, 4H), 7.03 (d, 1H), 3.72 (d, 1H), 3.51 (d, 1H), 3.41-3.35 (m, 1H), 3.06 (ddt, 1H), 2.86-2.69 (m, 6H), 2.65 (dd, 1H), 2.57-2.52 (m, 1H), 2.36 (s, 3H), 1.96-1.89 (m, 1H), 1.72-1.63 (m, 1H); LC-MS (ESI) m/z 413.1 [M+H]⁺.

### Example 243: trans-N-(((3R)-6-Bromo-2-methyl-1,2,3,4-tetrahydroisoquinolin-3-yl)methyl)-2-phenylcyclopropane-1-carboxamide (243)

The compound was prepared by general procedure i1 with Six (300 mg, 1.18 mmol), trans-2-phenyl-1-cyclopropanecarboxylic acid (172 mg, 1.06 mmol), 1-(3-dimethylaminopropyl)-3-ethylcarbodiimide hydrochloride (338 mg, 1.76 mmol), 1-hydroxybenzotriazole hydrate (238 mg, 1.76 mmol) and N,N-diisopropylethylamine (720 μL, 4.12 mmol) in dichloromethane (23 mL). The crude was purified by flash column chromatography (SiO₂, 3% methanol in dichloromethane) to give 243 as a white solid (272 mg, 64%). ¹H NMR (600 MHz, DMSO-d₆) δ 8.12 (br q, 1H), 7.30-7.23 (m, 4H), 7.16 (td, 1H), 7.13-7.09 (m, 2H), 7.00 (d, 1H), 3.71 (d, 1H), 3.49 (d, 1H), 3.38-3.33 (m overlapped with H₂O, 1H), 3.14-3.06 (m, 1H), 2.76 (dt, 1H), 2.73-2.68 (m, 1H), 2.68-2.62 (m, 1H), 2.34 (s, 1.5H), 2.33 (s, 1.5H), 2.27-2.23 (m, 1H), 2.00-1.95 (m, 1H), 1.40-1.34 (m, 1H), 1.17 (dddd, 1H); LC-MS (ESI) m/z 399.1 [M+H]⁺.

### Example 244: N-(((3S)-6-Bromo-2-methyl-1,2,3,4-tetrahydroisoquinolin-3-yl)methyl)-2,3-dihydro-1H-indene-2-carboxamide (244)

The compound was prepared by general procedure i1 with hix (104 mg, 0.408 mmol), 2-indanecarboxylic acid (59 mg, 0.367 mmol), 1-(3-dimethylaminopropyl)-3-ethylcarbodiimide hydrochloride (117 mg, 0.611 mmol), 1-hydroxybenzotriazole hydrate (83 mg, 0.611 mmol) and N,N-diisopropylethylamine (250 μL, 1.43 mmol) in dichloromethane (4 mL). The crude was purified by flash column chromatography (SiO₂, 3% methanol in dichloromethane) to give 244 as a white solid (120 mg, 82%). ¹H NMR (400 MHz, DMSO-d₆) δ 7.93 (br t, 1H), 7.33-7.27 (m, 2H), 7.21-7.15 (m, 2H), 7.15-7.09 (m, 2H), 7.05-7.01 (m, 1H), 3.73 (d, 1H), 3.51 (d, 1H), 3.37 (dt, 1H), 3.24-3.16 (m, 1H), 3.12-3.06 (m, 1H), 3.06-3.01 (m, 4H), 2.83-2.70 (m, 2H), 2.65 (dd, 1H), 2.36 (s, 3H); LC-MS (ESI) m/z 399.1 [M+H]⁺.

### Example 245: N-(((3S)-6-Bromo-2-methyl-1,2,3,4-tetrahydroisoquinolin-3-yl)methyl)-1,2,3,4-tetrahydronaphthalene-2-carboxamide (245)

The compound was prepared by general procedure i1 with hix (104 mg, 0.408 mmol), 1,2,3,4-tetrahydronaphthalene-2-carboxylic acid (65 mg, 0.367 mmol), 1-(3-dimethylaminopropyl)-3-ethylcarbodiimide hydrochloride (117 mg, 0.611 mmol), 1-hydroxybenzotriazole hydrate (83 mg, 0.611 mmol) and N,N-diisopropylethylamine (250 μL, 1.43 mmol) in dichloromethane (4 mL). The crude was purified by flash column chromatography (SiO₂, 3% methanol in dichloromethane) to give 245 as a yellow syrup (124 mg, 82%). ¹H NMR (400 MHz, DMSO-d₆) δ 7.87 (br t, 1H), 7.33-7.28 (m, 2H), 7.09-7.05 (m, 4H), 7.03 (d, 1H), 3.73 (d, 1H), 3.51 (d, 1H), 3.43-3.34 (m, 1H), 3.11-3.00 (m, 1H), 2.88-2.69 (m, 6H), 2.65 (dd, 1H), 2.58-2.52 (m, 1H), 2.36 (s, 3H), 1.98-1.88 (m, 1H), 1.75-1.59 (m, 1H); LC-MS (ESI) m/z 413.1 [M+H]⁺.

### Example 246: N-(((3R)-7-Fluoro-2-methyl-1,2,3,4-tetrahydroisoquinolin-3-yl)methyl)-4-(trifluoromethyl)benzamide (246)

The compound was prepared by general procedure i1 with kix (70 mg, 0.36 mmol), 4-(trifluoromethyl)benzoic acid (65.1 mg, 0.342 mmol), EDCI (89.5 mg, 0.468 mmol), HOBt (53.6 mg, 0.396 mmol) and DIPEA (89 μL, 0.541 mmol) in CH₂Cl₂ (4 mL), stirred at room temperature for 17 h. After workup, the crude was purified by Isco Combi-Flash Companion column chromatography (0-5% methanol in CH₂Cl₂) to give 246 (40.9 mg, 31%). ¹H NMR (400 MHz, DMSO-d₆) δ 8.69 (br t, 1H), 8.04 (d, 2H), 7.86 (d, 2H), 7.14 (dd, 1H), 7.01-6.88 (m, 2H), 3.79 (d, 1H), 3.62 (d, 1H), 3.59-3.52 (m, 1H), 3.23 (ddd, 1H), 2.96 (ddd, 1H), 2.81 (dd, 1H), 2.66 (dd, 1H), 2.41 (s, 3H); LC-MS (ESI) m/z 367.1 [M+H]⁺.

### Example 247: 3,4-Dichloro-N-(((3R)-7-fluoro-2-methyl-1,2,3,4-tetrahydroisoquinolin-3-yl)methyl)benzamide (247)

The compound was prepared by general procedure i1 with kix (70 mg, 0.36 mmol), 3,4-dichlorobenzoic acid (65.1 mg, 0.342 mmol), EDCI (89.5 mg, 0.468 mmol), HOBt (53.6 mg, 0.396 mmol) and DIPEA (89 μL, 0.541 mmol) in CH₂Cl₂ (4 mL), stirred at room temperature for 17 h. After workup, the crude was purified by Isco Combi-Flash Companion column chromatography (0-5% methanol in CH₂Cl₂) to give 247 (40.9 mg, 31%). ¹H NMR (400 MHz, DMSO-d₆) δ 8.64 (br t, 1H), 8.07 (d, 1H), 7.83 (dd, 1H), 7.76 (d, 1H), 7.14 (dd, 1H), 7.03-6.88 (m, 2H), 3.78 (d, 1H), 3.61 (d, 1H), 3.54 (dt, 1H), 3.21 (ddd, 1H), 2.94 (ddd, 1H), 2.81 (dd, 1H), 2.64 (dd, 1H), 2.40 (s, 3H); LC-MS (ESI) m/z 368.1 [M+H]⁺.

### Example 248: N-(((3S)-7-Fluoro-2-methyl-1,2,3,4-tetrahydroisoquinolin-3-yl)methyl)-4-(trifluoromethyl)benzamide (248)

The compound was prepared by general procedure i1 Iix (68.5 mg, 0.353 mmol), 4-(trifluoromethyl)benzoic acid (63.7 mg, 0.335 mmol), EDCI (87.6 mg, 0.458 mmol), HOBt (52.4 mg, 0.388 mmol) and DIPEA (87 μL, 0.529 mmol) in CH₂Cl₂ (4 mL), stirred at room temperature for 17 h. After workup, the crude was purified by Isco Combi-Flash Companion column chromatography (0-5% methanol in CH₂Cl₂) to give 248 (49.5 mg, 38%). ¹H NMR (400 MHz, DMSO-d₆) δ 8.69 (br t, 1H), 8.04 (d, 2H), 7.86 (d, 2H), 7.14 (dd, 1H), 7.03-6.89 (m, 2H), 3.79 (d, 1H), 3.62 (d, 1H), 3.59-3.53 (m, 1H), 3.23 (ddd, 1H), 2.96 (ddd, 1H), 2.81 (dd, 1H), 2.66 (dd, 1H), 2.41 (s, 3H); LC-MS (ESI) m/z 367.1 [M+H]⁺.

### Example 249: 3,4-Dichloro-N-(((3S)-7-fluoro-2-methyl-1,2,3,4-tetrahydroisoquinolin-3-yl)methyl)benzamide (249)

The compound was prepared by general procedure i1 with Iix (68.5 mg, 0.353 mmol), 3,4-dichlorobenzoic acid (64 mg, 0.335 mmol), EDCI (87.6 mg, 0.458 mmol), HOBt (52.4 mg, 0.388 mmol) and DIPEA (87 μL, 0.529 mmol) in CH₂Cl₂ (4 mL), stirred at room temperature for 17 h. After workup, the crude was purified by Isco Combi-Flash Companion column chromatography (0-5% methanol in CH₂Cl₂) to give 249 (35.1 mg, 27%). ¹H NMR (400 MHz, DMSO-d₆) δ 8.64 (br t, 1H), 8.07 (d, 1H), 7.83 (dd, 1H), 7.77 (d, 1H), 7.14 (dd, 1H), 7.01-6.90 (m, 2H), 3.78 (d, 1H), 3.61 (d, 1H), 3.54 (dt, 1H), 3.21 (ddd, 1H), 2.94 (ddd, 1H), 2.81 (dd, 1H), 2.64 (dd, 1H), 2.40 (s, 3H); LC-MS (ESI) m/z 368.0 [M+H]⁺.

### Example 250: N-(((3R)-7-Chloro-2-methyl-1,2,3,4-tetrahydroisoquinolin-3-yl)methyl)-4-ethylbenzamide (250)

The compound was prepared by general procedure i1 with mix (30 mg, 0.142 mmol), 4-ethylbenzoic acid (20.3 mg, 0.135 mmol), EDCI (35.4 mg, 0.185 mmol), HOBt (21.2 mg, 0.157 mmol) and DIPEA (35.3 μL, 0.214 mmol) in CH₂Cl₂ (3 mL), stirred at room temperature for 17 h. After workup, the crude was purified by Isco Combi-Flash Companion column chromatography (0-3% methanol in CH₂Cl₂) to give 250 (35.1 mg, 27%). ¹H NMR (400 MHz, Acetone-d₆) δ 7.81 (d, 2H), 7.61 (br s, 1H), 7.30 (d, 2H), 7.19-7.09 (m, 3H), 3.86 (d, 1H), 3.72-3.60 (m, 2H), 3.44 (ddd, 1H), 3.02 (ddd, 1H), 2.88 (dd, 1H), 2.77 (dd, 1H), 2.69 (q, 2H), 2.47 (s, 3H), 1.23 (t, 3H); LC-MS (ESI) m/z 343.1 [M+H]⁺.

### Example 251: N-(((3S)-7-Chloro-2-methyl-1,2,3,4-tetrahydroisoquinolin-3-yl)methyl)-4-(trifluoromethyl)benzamide (251)

The compound was prepared by general procedure i1 with nix (120 mg, 0.570 mmol), 4-ethylbenzoic acid (100 mg, 0.541 mmol), EDCI (140 mg, 0.74 mmol), HOBt (84.6 mg, 0.626 mmol) and DIPEA (140 μL, 0.854 mmol) in CH₂Cl₂ (4 mL), stirred at room temperature for 17 h. After workup, the crude was purified by Isco Combi-Flash Companion column chromatography (0-3% methanol in CH₂Cl₂) to give 251 (81.3 mg, 37%). ¹H NMR (400 MHz, DMSO-d₆) δ 8.69 (br t, 1H), 8.03 (d, 2H), 7.86 (d, 2H), 7.22-7.09 (m, 3H), 3.79 (d, 1H), 3.62 (d, 1H), 3.59-3.52 (m, 1H), 3.23 (ddd, 1H), 2.96 (ddd, 1H), 2.82 (dd, 1H), 2.66 (dd, 1H), 2.41 (s, 3H); LC-MS (ESI) m/z 383.1 [M+H]⁺.

### Example 252: N-(((3S)-7-Chloro-2-methyl-1,2,3,4-tetrahydroisoquinolin-3-yl)methyl)-4-ethylbenzamide (252)

The compound was prepared by general procedure i1 with nix (130 mg, 0.617 mmol), 4-ethylbenzoic acid (88 mg, 0.586 mmol), EDCI (150 mg, 0.802 mmol), HOBt (91.7 mg, 0.679 mmol) and DIPEA (153 μL, 0.925 mmol) in CH₂C₁₂ (4 mL), stirred at room temperature for 17 h. After workup, the crude was purified by Isco Combi-Flash Companion column chromatography (0-3% methanol in CH₂Cl₂) to give 252 (120 mg, 57%). ¹H NMR (400 MHz, DMSO-d₆) δ 8.35 (br t, 1H), 7.77 (d, 2H), 7.30 (d, 2H), 7.23-7.09 (m, 3H), 3.78 (d, 1H), 3.60 (d, 1H), 3.53 (dt, 1H), 3.19 (ddd, 1H), 2.94 (ddd, 1H), 2.80 (dd, 1H), 2.73-2.58 (m, 3H), 2.40 (s, 3H), 1.19 (t, 3H); LC-MS (ESI) m/z 343.1 [M+H]⁺.

### Example 253

### Animals

Male wild-type C₅₇BL/6 (B6) mice (25-30 g) were randomized to an experimental group. The protocol was approved by the Institutional Animal Care and Use Committee of the National Health Research Institutes, Taiwan. All Animal studies were conducted in accordance with the Policies on the Use of Animals in Neuroscience Research and the ethical guidelines for investigations of experimental pain in conscious animals established by the International Association for the Study of Pain. Animals were kept in a temperature-controlled animal room with a 12:12 h light-dark cycle.

### Materials

Compounds were synthesized following the procedures described above. Regents were purchased via commercial sources. Morphine hydrochloride was obtained from the Food and Drug Administration, Ministry of Health and Welfare, Taipei, Taiwan.

### FLIPR® Calcium Assay

One day before the assay, Greiner μClear® half area black 96-well assay plates CHO-K1/MOR/Gα15 cells were suspended in a F12 complete medium and plated at a density of ˜3.7×10⁴ cells/well in 100 μL medium. After 6-7 hours, 100 μL/well complete medium was removed, and added 100 μL/well F12 no-serum medium to starvation the cells overnight. Cells were incubated in a humidified atmosphere of 10% CO₂ at 37° C. overnight so as to reach an 80-90% confluent cell monolayer before assay. At the day of assay, 100 μL medium/well was removed from plate. To each well, 40 μL FLIPR® calcium assay reagent dissolved in 1× assay buffer (HBSS: KCl 5 mM, KH₂PO₄ 0.3 mM, NaCl 138 mM, NaHCO₃ 4 mM, Na₂HPO₄ 0.3 mM, d-glucose 5.6 mM, with additional 20 mM HEPES and 13 mM CaCl₂). pH 7.4), and the plate was incubated at 37° C. for 1 h. Compounds and other reagents were dissolved in the assay buffer. Using a FlexStation® III (Molecular Devices Corp.), the [Ca²⁺]ᵢ fluorescence increases after robotic injections of compounds or other reagents were monitored every 1.52 s interval with excitation wavelength at 485 nm and with emission wavelength at 525 nm. The [Ca²⁺]ᵢ fluorescence was measured up to 360 s after agonist injection. The fluorescence intensity from 6 to 12 wells of cells were averaged and the relative amount of [Ca²⁺]ᵢ release was determined by integrating the AUC of the [Ca²⁺]ᵢ fluorescence averages.

All tested compounds showed an EC₅₀ value, and all the tested compounds showed an AUC more than 1000. Data was shown above.

### CAMP Assay (Cyclic Adenosine Monophosphate Assay)

CHO-K1-NOP cells (72,000 cells per well) were seeded into 96-well flat bottom clear plates (GIBCO) with 150 μL of DMEM per well. An additional 50 μL per well of test compounds diluted in HBSS was added, along with forskolin and 3-isobutyl-1-methylxanthine at final concentrations of 1 μM and 500 μM, respectively.

After 30 minutes of incubation at room temperature, intracellular cyclic adenosine monophosphate (cAMP) levels were quantified using the LANCE Ultra CAMP Assay Kit (Cat #TRF0263; PerkinElmer). One hour later, plate fluorescence was measured using a Victor 2 plate reader, with excitation at 330 nm and emission detection at 615 nm and 665 nm.

Data are expressed as the percentage inhibition of cAMP accumulation, calculated using the formula:

$$( 1 - c A M {P}_{t r e a t m e n t \& acute compounds with forskolin} / {cAMp}_{forskolin} ) \times 100 \% .$$

All the tested compounds showed either an EC₅₀ value or a % of inhibition value. Data was shown in the DETAILED DESCRIPTION OF THE INVENTION section.

### Tail-Flick Test

Drug-induced antinociception against acute thermal pain was evaluated using the Tail-Flick Analgesia Meter (Columbia Instruments, Columbus, OH, USA). The basal latency was recorded before treatment and mice with a basal latency between 2.5-3.5 s was collected and randomly divided into each group. The tail-flick latencies were recorded at different time points after intravenous (i.v.), intraperitoneal (i.p.), subcutaneous (s.c.), or oral administration of drugs. The s.c. and oral dosing solution of test compounds were prepared in 5% DMA, 5% solutol, and 4.5% dextrose or 4.5% captisol in d.d. H₂O. To avoid tissue damage, the cutoff time was 10 sec. The antinociceptive effect was defined as the difference between the tail-flick latency and the basal latency.

The activities of tail-flick test are presented in two doses, i.e., ++: 3 mg/kg (subcutaneous) and +: 10 mg/kg (subcutaneous) active (tail-flick latency-basal latency >3 sec). See Table 5 below.

TABLE 5

| No. | Tail-flick test |
|---|---|
| 1 | ++ |
| 5 | + |
| 7 | + |
| 12 | + |
| 14 | ++ |
| 15 | ++ |
| 16 | ++ |
| 17 | ++ |
| 19 | ++ |
| 20 | ++ |
| 21 | ++ |
| 26 | + |
| 27 | + |
| 28 | + |
| 36 | ++ |
| 41 | ++ |
| 42 | ++ |
| 53 | ++ |
| 61 | + |
| 62 | + |
| 63 | + |
| 64 | + |
| 65 | ++ |
| 67 | ++ |
| 68 | ++ |
| 69 | ++ |
| 70 | ++ |
| 71 | ++ |
| 74 | ++ |
| 75 | ++ |
| 78 | + |
| 80 | ++ |
| 87 | + |
| 99 | + |
| 97 | + |
| 102 | ++ |
| 103 | ++ |
| 104 | ++ |
| 105 | ++ |
| 111 | + |
| 130 | ++ |
| 131 | ++ |
| 163 | ++ |

### Statistical Analysis

All in vitro and in vivo experiments were repeated multiple times to ensure the reliability of the individual values. The sample size used in all experiments was based on previous experience. No samples, mice, or data points were excluded from the reported analysis. Investigators were blinded to the test conditions. In all experiments, an individual administered drugs to the cells or animals, and another individual who was blinded to the drug administered observed the response and analyzed the data (GraphPad Prism version 5.0, GraphPad Software, San Diego, CA, USA).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the compounds of this invention also can be made, screened for their modulating activities to opioid receptor and treating opioid receptor associated conditions. Thus, other embodiments are also within the claims.

What is claimed is:

1. A compound of Formula (II): 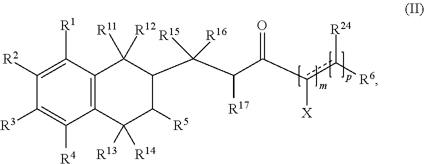
in which
each of R¹ to R⁴, independently, is H, halo, cyano, amino, nitro, thio, C₁₋₆ alkyl, or C₁₋₆ alkoxy;
R⁵ is C₁₋₆ alkyl, C₃₋₈ cycloalkyl, or C₁₋₆ alkenyl;
R⁶ is C₁₋₆ alkyl, C₃₋₈ cycloalkyl, C₂₋₈ heterocycloalkyl, C₆₋₁₄ aryl, C₆₋₁₄ cycloalkylaryl, C₆₋₁₄ aralkyl, C₂₋₁₃ heteroaryl, or 6 to 16-membered heterocycloalkylaryl;
each of R¹¹ to R¹⁶, independently, is H, halo, hydroxyl, cyano, amino, nitro, thio, carboxyl, C₁₋₆ alkyl, or C₁₋₆ alkoxy;
R¹⁷ is H, C₁₋₆ alkyl, C₃₋₈ cycloalkyl, or C₁₋₆ alkenyl;
X is absent, H, or CR¹⁸R¹⁹R²⁰, or X and R⁶, together with the carbon atoms they bond to, form a five or six-membered cycloalkyl- or heterocycloalkyl-fused phenyl, or X and R²⁴, together with the carbon atoms they bond to, form cyclopropylene;
each of R¹⁸ to R²⁰, independently, is H, halo, hydroxyl, thio, cyano, amino, nitro, carboxyl, C₁₋₆ alkyl, or C₁₋₆ alkoxy;
R²⁴ is absent, H, halo, hydroxyl, cyano, amino, nitro, thio, carboxyl, C₁₋₆ alkyl, C₃₋₈ cycloalkyl, or C₁₋₆ alkoxy;
each of amino, thio, alkyl, alkoxy, and alkenyl is unsubstituted or substituted with one or more of halo, hydroxyl, thio, cyano, amino, nitro, carboxyl, and aryl;
each of cycloalkyl, heterocycloalkyl, cycloalkylaryl, aralkyl, heteroaryl, heterocycloalkylaryl, and cyclopropylene is unsubstituted or substituted with one or more of halo, hydroxyl, thio, cyano, amino, nitro, carboxyl, alkyl, alkoxy, and aryl;
aryl is unsubstituted or substituted with one or more of halo, thio, cyano, amino, nitro, carboxyl, alkyl, and aryl;
each of the five or six-membered cycloalkyl- or heterocycloalkyl-fused phenyl is unsubstituted or substituted with one or more of halo, hydroxyl, thio, cyano, amino, nitro, carboxyl, alkyl, alkoxy, and aryl;
each of m and n, independently, is 0 or 1; and [symbol]
is a single bond or double bond.

2. The compound of claim 1, wherein the compound is a compound of Formula (III): 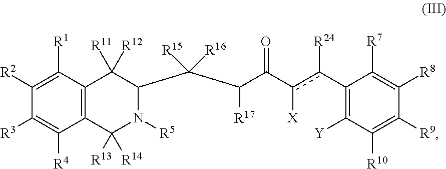
in which
each of R⁷ to R¹⁰, independently, is H, halo, thio, cyano, amino, nitro, carboxyl, C₁₋₆ alkyl, or C₁₋₆ alkoxy;
R¹⁷ is H;
X is H or CR¹⁸R¹⁹R²⁰, Y is a H or CR²¹R²²R²³, or X and Y, together with the carbon atoms they bond to, form a phenyl ring, a five- or six-membered cycloalkyl ring, or a five or six-membered heterocycloalkyl ring;
each of R²¹ to R²³ is H, halo, hydroxyl, thio, cyano, amino, nitro, carboxyl, C₁₋₆ alkyl, or C₁₋₆ alkoxy;
R²⁴ is H; and
phenyl is unsubstituted or substituted with one or more of halo, hydroxyl, thio, cyano, amino, nitro, carboxyl, alkyl, alkoxy, and aryl.

3. A compound of Formula (IV): 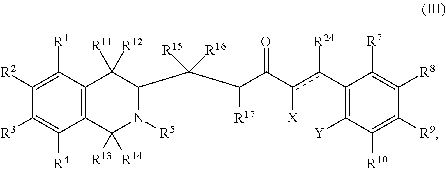
in which
each of R¹ to R⁴, R⁷ to R¹⁶, and R²⁴, independently, is H, halo, hydroxyl, cyano, amino, nitro, thio, carboxyl, C₁₋₆ alkyl, or C₁₋₆ alkoxy;
R⁵ is C₁₋₆ alkyl, C₃₋₈ cycloalkyl, or C₁₋₆ alkenyl;
R¹⁷ is H, C₁₋₆ alkyl, C₃₋₈ cycloalkyl, or C₁₋₆ alkenyl;
X is S, CR¹⁸R¹⁹, or NR¹⁸;
Y is a bond or CR²¹R²²;
each of R¹⁸, R¹⁹, R²¹, and R²², independently, is H, halo, hydroxyl, thio, cyano, amino, nitro, carboxyl, C₁₋₆ alkyl, or C₁₋₆ alkoxy;
each of amino, thio, alkyl, alkoxy, alkenyl, and cycloalkyl, independently, is unsubstituted or substituted with one or more of halo, hydroxyl, thio, cyano, amino, nitro, carboxyl, alkyl, alkoxy, and aryl, and [symbol]
is a single bond or double bond.

4. The compound of claim 1, wherein the compound is a compound of Formula (V): 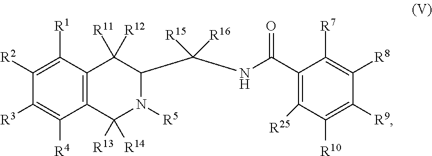
in which
each of R¹ to R⁴, independently, is H, halo, cyano, amino, nitro, thio, carboxyl, C₁₋₆ alkyl, or C₁₋₆ alkoxy;
R⁵ is methyl, C₁₋₆ alkoxyalkyl, or C₁₋₆ alkenyl;
each of R⁷ to R¹⁶ and R²⁵, independently, is H, halo, hydroxyl, cyano, amino, nitro, thio, carboxyl, C₁₋₆ alkyl, or C₁₋₆ alkoxy; and
alkoxyalkyl is unsubstituted or substituted with one or more of halo, hydroxyl, thio, cyano, amino, nitro, carboxyl, and aryl.

5. The compound of claim 1, wherein each of R¹ to R⁴, independently, is H, halo, cyano, amino, nitro, thio, or C₁₋₆ alkyl, preferably H, F, Br, Cl, methyl, or ethyl.

6. The compound of claim 5, wherein R¹ and R², independently, is H, F, Cl, Br, or methyl; R³ is H or F; and R⁴ is H.

7. The compound of claim 1, wherein R⁵ is methyl, prop-2-en-yl, 3-methylbut-2-en-1-yl, or 2-methoxyethyl.

8. The compound of claim 2, wherein each of R⁷ to R¹⁰, independently, is H, F, Cl, CF₃, ethyl, or ethylthio.

9. The compound of claim 1, wherein each of R¹¹ to R¹⁶ is H.

10. The compound of claim 1, wherein each m and n is 1 and R¹⁷ is H.

11. The compound of claim 1, wherein each of X and R²⁴ is H or X and R²⁴, together with the carbon atoms they bond to, form cyclopropylene; and
R⁶ is

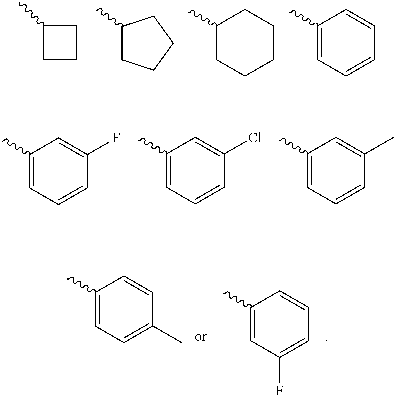

12. The compound of claim 1, wherein X is H, NH, or CH₂.

13. The compound of claim 2, wherein Y is H, a bond, or CH₂.

14. The compound of claim 1, wherein R⁶ is

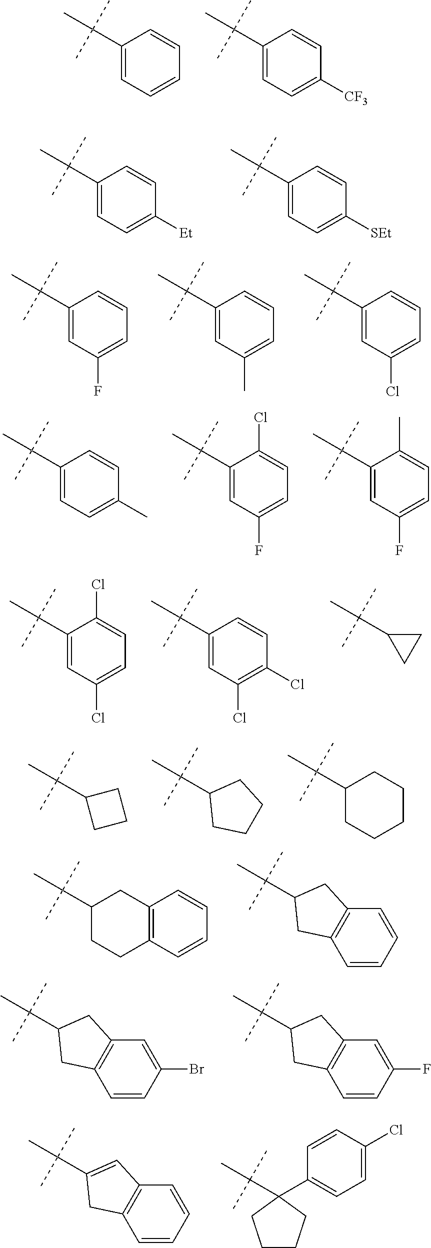

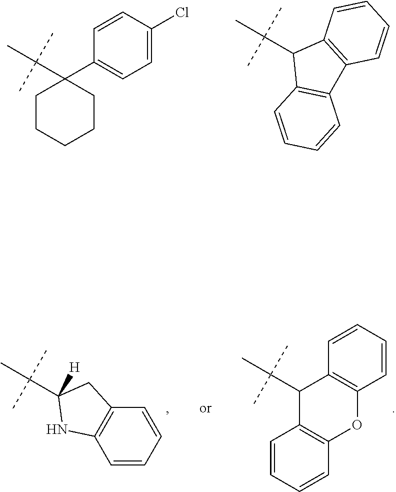

15. The compound of claim 1, wherein the stereoisomeric configuration of carbon-3 is R or S.

16. The compound of claim 1, wherein the compound is one of Compounds 1-252.

17. A pharmaceutical composition for treating an opioid receptor-associated condition in a subject comprising a pharmaceutically acceptable carrier and a compound of claim 1.

18. A method of treating an opioid receptor-associated condition in a subject comprising administering to the subject in need thereof a pharmaceutically effective amount of a compound of claim 1.

19. The method of 18, wherein the opioid receptor-associated condition is pain, immune disease, esophageal reflux, diarrhea, anxiety, or heroin addiction.

20. The method of claim 19, wherein the opioid receptor-associated condition is cancer pain, post operative pain, low back pain, rheumatoid arthritis pain, osteoarthritis pain, neuropathic pain, or fibromyalgia pain.
